(19) United States
(12) Patent Application Publication
(10) Pub. No.: US 2019/0144907 A1
(43) Pub. Date: May 16, 2019

(54) PRODUCTION OF STEVIOL GLYCOSIDES IN RECOMBINANT HOSTS

(71) Applicant: Evolva SA, Reinach (CH)

(72) Inventors: Iver Klavs Riishede Hansen, Copenhagen (DK); Veronique Douchin, Frederiksberg (DK); Jens Houghton-Larsen, Birkeroed (DK); Esben Halkjaer Hansen, Frederiksberg C (DK); Angelika Semmler, Copenhagen (DK); Laura Occhipinti, Zurich (CH); Kim Olsson, Copenhagen (DK)

(21) Appl. No.: 16/098,305

(22) PCT Filed: May 16, 2017
(86) PCT No.: PCT/EP2017/061774

§ 371 Date: Nov. 1, 2018

Related U.S. Application Data
(60) Provisional application No. 62/337,213, filed on May 16, 2016.

(51) Int. Cl. C12P 19/56; C12N 15/70; C12N 15/81

(52) U.S. Cl. CPC C12P 19/56; C12N 15/81; C12N 15/70

(57) ABSTRACT

The invention relates to recombinant microorganisms and methods for producing steviol glycosides, glycosides of steviol precursors, and steviol glycoside precursors.

### BACKGROUND OF THE INVENTION

### Field of the Invention

This disclosure relates to recombinant production of steviol glycosides, glycosides of steviol precursors, and steviol glycoside precursors in recombinant hosts. In particular, this disclosure relates to production of steviol glycosides comprising steviol-13-O-glucoside (13-SMG), steviol-19-O-glucoside (19-SMG), steviol-1,2-bioside, 1,2-stevioside, rubusoside, rebaudioside A (RebA), rebaudioside B (RebB), rebaudioside D (RebD), rebaudioside M (RebM), mono-glycosylated ent-kaurenoic acids, di-glycosylated ent-kaurenoic acids, tri-glycosylated ent-kaurenoic acids, tri-glycosylated ent-kaurenols, tri-glycosylated steviol glycosides, tetra-glycosylated steviol glycosides, penta-glycosylated steviol glycosides, hexa-glycosylated steviol glycosides, hepta-glycosylated steviol glycosides, or isomers thereof in recombinant hosts.

### Description of Related Art

Sweeteners are well known as ingredients used most commonly in the food, beverage, or confectionary industries. The sweetener can either be incorporated into a final food product during production or for stand-alone use, when appropriately diluted, as a tabletop sweetener or an at-home replacement for sugars in baking. Sweeteners include natural sweeteners such as sucrose, high fructose corn syrup, molasses, maple syrup, and honey and artificial sweeteners such as aspartame, saccharine, and sucralose. Stevia extract is a natural sweetener that can be isolated and extracted from a perennial shrub, Stevia rebaudiana. Stevia is commonly grown in South America and Asia for commercial production of stevia extract. Stevia extract, purified to various degrees, is used commercially as a high intensity sweetener in foods and in blends or alone as a tabletop sweetener.

Chemical structures for several steviol glycosides are shown in FIG. 1, including the diterpene steviol and various steviol glycosides. Extracts of the Stevia plant generally comprise steviol glycosides that contribute to the sweet flavor, although the amount of each steviol glycoside often varies, inter alia, among different production batches.

As recovery and purification of steviol glycosides from the Stevia plant have proven to be labor intensive and inefficient, there remains a need for a recombinant production system that can accumulate high yields of desired steviol glycosides, such as RebD and RebM. There also remains a need for improved production of steviol glycosides in recombinant hosts for commercial uses.

### SUMMARY OF THE INVENTION

It is against the above background that the present invention provides certain advantages and advancements over the prior art.

Although this invention as disclosed herein is not limited to specific advantages or functionalities, the invention provides a recombinant host cell capable of producing one or more steviol glycosides and/or glycosylated steviol precursors, or a composition thereof, comprising:
(a) a gene encoding a polypeptide capable of glycosylating steviol or a steviol glycoside at its C-19 carboxyl position;
(b) a gene encoding a polypeptide capable of glycosylating steviol or a steviol glycoside at its C-13 hydroxyl position;
(c) a gene encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside; and/or
(d) a gene encoding a polypeptide capable of glycosylating a steviol precursor at its C-19 carboxyl or C-19 hydroxyl position;

wherein at least one of the genes is a recombinant gene.

In one aspect of the recombinant host cell disclosed herein:
(a) the polypeptide capable of glycosylating steviol or a steviol glycoside at its C-19 carboxyl position is a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, and/or a UGT74F2-like UGT polypeptide;
(b) the polypeptide capable of glycosylating steviol or a steviol glycoside at its C-13 hydroxyl position is a UGT73C1 polypeptide, a UGT73C3 polypeptide, a UGT73C5 polypeptide, a UGT73C6 polypeptide, a UGT73C7 polypeptide, a UGT73E1 polypeptide, and/or a UGT76E12 polypeptide;
(c) the polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside is a UGT73C6 polypeptide, a CaUGT3 polypeptide, a UN32491 polypeptide, and/or a UN1671 polypeptide; and/or
(d) the polypeptide capable of glycosylating a steviol precursor at its C-19 carboxyl or C-19 hydroxyl position is a UGT73C1 polypeptide, a UGT73C3 polypeptide, a UGT73C5 polypeptide, a UGT73C6 polypeptide, a UGT73E1 polypeptide, a UGT74D1 polypeptide, a UGT75B1 polypeptide, a UGT75L6 polypeptide, a UGT76E12 polypeptide, a Olel polypeptide, a UGT5 polypeptide, a SA Gtase, a UDPG1 polypeptide, a UGT74F1 polypeptide, a UGT75D1 polypeptide, a UGT84B2 polypeptide, a CaUGT2 polypeptide, and/or a UGT74F2-like UGT polypeptide.

In one aspect of the recombinant host cell disclosed herein: the UGT73C1 polypeptide comprises a polypeptide having at least 60% identity to an amino acid sequence set forth in SEQ ID NO:127, the UGT73C3 polypeptide comprises a polypeptide having at least 60% identity to an amino acid sequence set forth in SEQ ID NO:133, the UGT73C5 polypeptide comprises a polypeptide having at least 60% identity to an amino acid sequence set forth in SEQ ID NO:135, the UGT73C6 polypeptide comprises a polypeptide having at least 60% identity to an amino acid sequence set forth in SEQ ID NO:137, the UGT73E1 polypeptide comprises a polypeptide having at least 50% identity to an amino acid sequence set forth in SEQ ID NO:141, the UGT74D1 polypeptide comprises a polypeptide having at least 50% identity to an amino acid sequence set forth in SEQ ID NO:143, the UGT75B1 polypeptide comprises a polypeptide having at least 50% sequence identity to an amino acid sequence set forth in SEQ ID NO:145, the UGT75L6 polypeptide comprises a polypeptide having at least 60% sequence identity to an amino acid sequence set forth in SEQ ID NO:147, the UGT76E12 polypeptide comprises a polypeptide having at least 60% sequence identity to an amino acid sequence set forth in SEQ ID NO:153, the Olel polypeptide comprises a polypeptide having at least 55% identity to an amino acid sequence set forth in SEQ ID NO:177, the UGT5 polypeptide comprises a polypeptide having at least 65% identity to an amino acid sequence set forth in SEQ ID NO:181, the SA Gtase polypeptide comprises a polypeptide having at least 55% identity to an amino acid sequence set forth in SEQ ID NO:183, the UDPG1 polypeptide comprises a polypeptide having at least 50% sequence identity to an amino acid sequence set forth in SEQ ID NO:185, the UN1671 polypeptide comprises a polypeptide having at least 45% identity to an amino acid sequence set forth in SEQ ID NO:201, the UGT74F1 polypeptide comprises a polypeptide having at least 50% sequence identity to an amino acid sequence set forth in SEQ ID NO:203, the UGT75D1 polypeptide comprises a polypeptide having at least 50% sequence identity to an amino acid sequence set forth in SEQ ID NO:205, the UGT84B2 polypeptide comprises a polypeptide having at least 40% sequence identity to an amino acid sequence set forth in SEQ ID NO:207, the UGT74F2-like UGT polypeptide comprises a polypeptide having at least 55% identity to an amino acid sequence set forth in SEQ ID NO:211, the UGT73C7 polypeptide comprises a polypeptide having at least 60% identity to an amino acid sequence set forth in SEQ ID NO:139, the CaUGT3 polypeptide comprises a polypeptide having at least 50% identity to an amino acid sequence set forth in SEQ ID NO:169, the UN32491 polypeptide comprises a polypeptide having at least 50% identity to an amino acid sequence set forth in SEQ ID NO:199, and/or the CaUGT2 polypeptide comprises a polypeptide having at least 55% identity to an amino acid sequence set forth in SEQ ID NO:209.

In one aspect of the recombinant host cell disclosed herein, the recombinant host cell further comprises:
(a) a gene encoding a polypeptide capable of synthesizing geranylgeranyl pyrophosphate (GGPP) from farnesyl diphosphate (FPP) and isopentenyl diphosphate (IPP);
(b) a gene encoding a polypeptide capable of synthesizing ent-copalyl diphosphate from GGPP;
(c) a gene encoding an a polypeptide capable of synthesizing ent-kaurene from ent-copalyl diphosphate;
(d) a gene encoding a polypeptide capable of synthesizing ent-kaurenoic acid from ent-kaurene;
(e) a gene encoding a polypeptide capable of reducing cytochrome P450 complex;
(f) a gene encoding a polypeptide capable of synthesizing steviol from ent-kaurenoic acid;
(g) a gene encoding a polypeptide capable of glycosylating steviol or a steviol glycoside at its C-13 hydroxyl position thereof;
(h) a gene encoding a polypeptide capable of beta 1,3 glycosylation of the C3′ of the 13-O-glucose, 19-O-glucose, or both 13-O-glucose and 19-O-glucose of a steviol glycoside;
(i) a gene encoding a polypeptide capable of glycosylating steviol or a steviol glycoside at its C-19 carboxyl position; and/or
(k) a gene encoding a polypeptide capable of beta 1,2 glycosylation of the C2′ of the 13-O-glucose, 19-O-glucose, or both 13-O-glucose and 19-O-glucose of a steviol glycoside;

wherein at least one of the genes is a recombinant gene.

In one aspect of the recombinant host cell disclosed herein:
(a) the polypeptide capable of synthesizing GGPP comprises a polypeptide having at least 70% sequence identity to the amino acid sequence set forth in SEQ ID NO:20, SEQ ID NO:22, SEQ ID NO:24, SEQ ID NO:26, SEQ ID NO:28, SEQ ID NO:30, SEQ ID NO:32, or SEQ ID NO:116;
(b) the polypeptide capable of synthesizing ent-copalyl diphosphate comprises a polypeptide having at least 70% sequence identity to the amino acid sequence set forth in SEQ ID NO:34, SEQ ID NO:36, SEQ ID NO:38, SEQ ID NO:40, SEQ ID NO:42, or SEQ ID NO:120;
(c) the polypeptide capable of synthesizing ent-kaurene comprises a polypeptide having at least 70% sequence identity to the amino acid sequence set forth in SEQ ID NO:44, SEQ ID NO:46, SEQ ID NO:48, SEQ ID NO:50, or SEQ ID NO:52;
(d) the polypeptide capable of synthesizing ent-kaurenoic acid comprises a polypeptide having at least 70% sequence identity to the amino acid sequence set forth in SEQ ID NO:60, SEQ ID NO:62, SEQ ID NO:117, SEQ ID NO:66, SEQ ID NO:68, SEQ ID NO:70, SEQ ID NO:72, SEQ ID NO:74, or SEQ ID NO:76;
(e) the polypeptide capable of reducing cytochrome P450 complex comprises a polypeptide having at least 70% sequence identity to the amino acid sequence set forth in SEQ ID NO:78, SEQ ID NO:80, SEQ ID NO:82, SEQ ID NO:84, SEQ ID NO:86, SEQ ID NO:88, SEQ ID NO:90, SEQ ID NO:92;
(f) the polypeptide capable of synthesizing steviol comprises a polypeptide having at least 70% sequence identity to the amino acid sequence set forth in SEQ ID NO:94, SEQ ID NO:97, SEQ ID NO:100, SEQ ID NO:101, SEQ ID NO:102, SEQ ID NO:103, SEQ ID NO:104, SEQ ID NO:106, SEQ ID NO:108, SEQ ID NO:110, SEQ ID NO:112, or SEQ ID NO:114;
(g) the polypeptide capable of glycosylating steviol or a steviol glycoside at its C-13 hydroxyl position thereof comprises a polypeptide having at least 55% sequence identity to the amino acid sequence set forth in SEQ ID NO:7;
(h) the polypeptide capable of beta 1,3 glycosylation of the C3′ of the 13-O-glucose, 19-O-glucose, or both 13-O-glucose and 19-O-glucose of a steviol glycoside comprises a polypeptide having at least 50% sequence identity to the amino acid sequence set forth in SEQ ID NO:9;
(i) the polypeptide capable of glycosylating steviol or a steviol glycoside at its C-19 carboxyl position comprises a polypeptide having at least 55% sequence identity to the amino acid sequence set forth in SEQ ID NO:4; and/or
(k) the polypeptide capable of beta 1,2 glycosylation of the C2′ of the 13-O-glucose, 19-O-glucose, or both 13-O-glucose and 19-O-glucose of a steviol glycoside comprises a polypeptide having 80% or greater identity to the amino acid sequence set forth in SEQ ID NO:11; a polypeptide having 80% or greater identity to the amino acid sequence set forth in SEQ ID NO:13; or a polypeptide having at least 65% sequence identity to the amino acid sequence set forth in SEQ ID NO:16.

In one aspect of the recombinant host cell disclosed herein, expression of the one or more recombinant genes increases an amount of the one or more steviol glycosides and/or glycosylated steviol precursors, or a composition thereof accumulated by the cell relative to a corresponding host lacking the one or more recombinant genes.

In one aspect of the recombinant host cell disclosed herein, expression of the one or more recombinant genes increases the amount of the one or more steviol glycosides and/or glycosylated steviol precursors, or the composition thereof, accumulated by the cell by at least about 5%, at least about 10%, at least about 25%, at least about 50%, at least about 75%, or at least about 100% relative to a corresponding host lacking the one or more recombinant genes.

In one aspect of the recombinant host cell disclosed herein, expression of the one or more recombinant genes increases the amount of ent-kaurenoic acid+2Glc (#7), ent-kaurenoic acid+3Glc (isomer 1), ent-kaurenoic acid+3Glc (isomer 2), steviol-13-O-glucoside (13-SMG), Rebaudioside A (RebA), Rebaudioside B (RebB), Steviol+4Glc (#36), Steviol+6Glc (isomer 1), Steviol+7Glc (isomer 2), and/or ent-Kaurenol+3Glc (isomer 1 and/or isomer 2) accumulated by the cell relative to a corresponding host lacking the one or more recombinant genes.

In one aspect of the recombinant host cell disclosed herein, the one or more steviol glycosides and/or glycosylated steviol precursors are, or the composition thereof comprises, 13-SMG, steviol-19-O-glucoside (19-SMG), steviol-1,2-bioside, steviol-1,3-bioside, 1,2-stevioside, 1,3-stevioside, rubusoside, RebA, RebB, Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, a mono-glycosylated ent-kaurenoic acid, a di-glycosylated ent-kaurenoic acid, a tri-glycosylated ent-kaurenoic acid, a mono-glycosylated ent-kaurenols, a di-glycosylated ent-kaurenol, a tri-glycosylated ent-kaurenol, a tri-glycosylated steviol glycoside, a tetra-glycosylated steviol glycoside, a penta-glycosylated steviol glycoside, a hexa-glycosylated steviol glycoside, a hepta-glycosylated steviol glycoside, or an isomer thereof.

In one aspect of the recombinant host cell disclosed herein, the mono-glycosylated ent-kaurenoic acid comprises KA1.58 of Table 1 and/or the penta-glycosylated steviol comprises Compound 5.24 of Table 1.

In one aspect of the recombinant host cell disclosed herein, the recombinant host cell comprises a plant cell, a mammalian cell, an insect cell, a fungal cell, an algal cell, or a bacterial cell.

The invention also provides a method of producing in a cell culture one or more steviol glycosides and/or glycosylated steviol precursors, or a composition thereof, comprising growing the recombinant host cell disclosed herein in the cell culture, under conditions in which the genes are expressed, and wherein the one or more steviol glycosides and/or glycosylated steviol precursors, or the composition thereof is produced by the recombinant host cell.

In one aspect of the method disclosed herein, the genes are constitutively expressed and/or expression of the genes is induced.

In one aspect of the method disclosed herein, an amount of ent-kaurenoic acid+2Glc (#7), ent-kaurenoic acid+3Glc (isomer 1), ent-kaurenoic acid+3Glc (isomer 2), 13-SMG, RebA, RebB, Steviol+4Glc (#36), Steviol+6Glc (isomer 1), Steviol+7Glc (isomer 2), and/or ent-Kaurenol+3Glc (isomer 1 and/or isomer 2) accumulated by the recombinant host cell is increased by at least about 5% relative to a corresponding host lacking the one or more recombinant genes.

In one aspect, the method disclosed herein further comprises isolating from the cell cultures the one or more steviol glycosides and/or glycosylated steviol precursors or the composition thereof produced thereby.

In one aspect of the method disclosed herein, the isolating step comprises:
(a) providing the cell culture comprising the one or more steviol glycosides and/or glycosylated steviol precursors, or the composition thereof;
(b) separating a liquid phase of the cell culture from a solid phase of the cell culture to obtain a supernatant comprising the produced one or more steviol glycosides and/or glycosylated steviol precursors, or the composition thereof;
(c) providing one or more adsorbent resins, comprising providing the adsorbent resins in a packed column; and
(d) contacting the supernatant of step (b) with the one or more adsorbent resins in order to obtain at least a portion of the produced one or more steviol glycosides and/or glycosylated steviol precursors, or the composition thereof, thereby isolating the produced one or more steviol glycosides or the steviol glycoside composition;
or
(a) providing the cell culture comprising the one or more steviol glycosides and/or glycosylated steviol precursors, or the composition thereof;
(b) separating a liquid phase of the cell culture from a solid phase of the cell culture to obtain a supernatant comprising the produced one or more steviol glycosides and/or glycosylated steviol precursors, or the composition thereof;
(c) providing one or more ion exchange or ion exchange or reversed-phase chromatography columns; and
(d) contacting the supernatant of step (b) with the one or more ion exchange or ion exchange or reversed-phase chromatography columns in order to obtain at least a portion of the produced one or more steviol glycosides and/or glycosylated steviol precursors, or the composition thereof, thereby isolating the produced one or more steviol glycosides and/or glycosylated steviol precursors, or the composition thereof;
or
(a) providing the cell culture comprising the one or more steviol glycosides and/or glycosylated steviol precursors, or the composition thereof;
(b) separating a liquid phase of the cell culture from a solid phase of the cell culture to obtain a supernatant comprising the produced one or more steviol glycosides and/or glycosylated steviol precursors, or the composition thereof;
(c) crystallizing or extracting the produced one or more steviol glycosides and/or glycosylated steviol precursors, or the composition thereof, thereby isolating the produced one or more steviol glycosides and/or glycosylated steviol precursors, or the composition thereof.

In one aspect, the method disclosed herein further comprises recovering from the cell culture the one or more steviol glycosides and/or glycosylated steviol precursors or the composition thereof from the cell culture, wherein the cell culture is enriched for the one or more steviol glycosides and/or glycosides of a steviol precursor, or the composition thereof relative to a steviol glycoside composition from a Stevia plant and has a reduced level of Stevia plant-derived components relative to a plant-derived Stevia extract.

In one aspect of the method disclosed herein, the recovered one or more steviol glycosides and/or glycosylated steviol precursors, or the composition thereof are present in relative amounts that are different from a steviol glycoside composition recovered from a Stevia plant and have a reduced level of Stevia plant-derived components relative to a plant-derived Stevia extract.

The invention also provides a method for producing one or more steviol glycosides and/or glycosylated steviol precursors, or the composition thereof, comprising whole cell bioconversion of plant-derived or synthetic steviol, steviol precursors and/or steviol glycosides in a cell culture medium of a recombinant host using:
(a) a gene encoding a polypeptide capable of glycosylating steviol or a steviol glycoside at its C-19 carboxyl position;
(b) a gene encoding a polypeptide capable of glycosylating steviol or a steviol glycoside at its C-13 hydroxyl position;
(c) a gene encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside; and/or
(d) a gene encoding a polypeptide capable of glycosylating a steviol precursor at its C-19 carboxyl or C-19 hydroxyl position;

wherein at least one of the polypeptides is a recombinant polypeptide expressed in the recombinant host cell; and producing the one or more steviol glycosides and/or glycosylated steviol precursors, or the composition thereof, thereby.

In one aspect of the method disclosed herein:
(a) the polypeptide capable of glycosylating steviol or a steviol glycoside at its C-19 carboxyl position is a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, and/or a UGT74F2-like UGT polypeptide;
(b) the polypeptide capable of glycosylating steviol or a steviol glycoside at its C-13 hydroxyl position is a UGT73C1 polypeptide, a UGT73C3 polypeptide, a UGT73C5 polypeptide, a UGT73C6 polypeptide, a UGT73C7 polypeptide, a UGT73E1 polypeptide, and/or a UGT76E12 polypeptide;
(c) the polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside is a UGT73C6 polypeptide, a CaUGT3 polypeptide, a UN32491 polypeptide, and/or a UN1671 polypeptide; and/or
(d) the polypeptide capable of glycosylating a steviol precursor at its C-19 carboxyl or C-19 hydroxyl position is a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a UGT76E12 polypeptide, a Olel polypeptide, a UGT5 polypeptide, a SA Gtase, a UDPG1 polypeptide, a UGT74F1 polypeptide, a UGT75D1 polypeptide, a UGT84B2 polypeptide, and/or a UGT74F2-like UGT polypeptide.

In one aspect of the method disclosed herein, the UGT73C1 polypeptide comprises a polypeptide having at least 60% identity to an amino acid sequence set forth in SEQ ID NO:127, the UGT73C3 polypeptide comprises a polypeptide having at least 60% identity to an amino acid sequence set forth in SEQ ID NO:133, the UGT73C5 polypeptide comprises a polypeptide having at least 60% identity to an amino acid sequence set forth in SEQ ID NO:135, the UGT73C6 polypeptide comprises a polypeptide having at least 60% identity to an amino acid sequence set forth in SEQ ID NO:137, the UGT73E1 polypeptide comprises a polypeptide having at least 50% identity to an amino acid sequence set forth in SEQ ID NO:141, a UGT74D1 polypeptide comprises a polypeptide having at least 50% identity to an amino acid sequence set forth in SEQ ID NO:143, the UGT75B1 polypeptide comprises a polypeptide having at least 50% sequence identity to an amino acid sequence set forth in SEQ ID NO:145, the UGT75L6 polypeptide comprises a polypeptide having at least 60% sequence identity to an amino acid sequence set forth in SEQ ID NO:147, the UGT76E12 polypeptide comprises a polypeptide having at least 60% sequence identity to an amino acid sequence set forth in SEQ ID NO:153, the Olel polypeptide comprises a polypeptide having at least 55% identity to an amino acid sequence set forth in SEQ ID NO:177, the UGT5 polypeptide comprises a polypeptide having at least 65% identity to an amino acid sequence set forth in SEQ ID NO:181, the SA Gtase polypeptide comprises a polypeptide having at least 55% identity to an amino acid sequence set forth in SEQ ID NO:183, the UDPG1 polypeptide comprises a polypeptide having at least 50% sequence identity to an amino acid sequence set forth in SEQ ID NO:185, the UN1671 polypeptide comprises a polypeptide having at least 45% identity to an amino acid sequence set forth in SEQ ID NO:201, the UGT74F1 polypeptide comprises a polypeptide having at least 50% sequence identity to an amino acid sequence set forth in SEQ ID NO:203, the UGT75D1 polypeptide comprises a polypeptide having at least 50% sequence identity to an amino acid sequence set forth in SEQ ID NO:205, the UGT84B2 polypeptide comprises a polypeptide having at least 40% sequence identity to an amino acid sequence set forth in SEQ ID NO:207, the UGT74F2-like UGT polypeptide comprises a polypeptide having at least 55% identity to an amino acid sequence set forth in SEQ ID NO:211, the UGT73C7 polypeptide comprises a polypeptide having at least 60% identity to an amino acid sequence set forth in SEQ ID NO:139, the CaUGT3 polypeptide comprises a polypeptide having at least 50% identity to an amino acid sequence set forth in SEQ ID NO:169, the UN32491 polypeptide comprises a polypeptide having at least 50% identity to an amino acid sequence set forth in SEQ ID NO:199, or the CaUGT2 polypeptide comprises a polypeptide having at least 55% identity to an amino acid sequence set forth in SEQ ID NO:209.

In one aspect of the method disclosed herein, the recombinant host cell is a plant cell, a mammalian cell, an insect cell, a fungal cell, an algal cell or a bacterial cell.

The invention also provides an in vitro method for producing one or more steviol glycosides and/or glycosylated steviol precursors, or a composition thereof comprising adding:
(a) a UGT85C2 polypeptide having at least 55% identity to an amino acid sequence set forth in SEQ ID NO:7;
(b) a UGT76G1 polypeptide having at least 50% identity to an amino acid sequence set forth in SEQ ID NO:9;
(c) a UGT74G1 polypeptide having at least 55% identity to an amino acid sequence set forth in SEQ ID NO:4;
(d) a UGT91D2 functional homolog polypeptide comprising a UGT91D2e polypeptide having 90% or greater identity to an amino acid sequence set forth in SEQ ID NO:11 or a UGT91D2e-b polypeptide having 90% or greater identity to an amino acid sequence set forth in SEQ ID NO:13;
(e) a EUGT11 polypeptide having at least 65% identity to an amino acid sequence set forth in SEQ ID NO:16; and/or
(f) a UGT73C1 polypeptide comprises a polypeptide having at least 60% identity to an amino acid sequence set forth in SEQ ID NO:127, a UGT73C3 polypeptide comprises a polypeptide having at least 60% identity to an amino acid sequence set forth in SEQ ID NO:133, a UGT73C5 polypeptide comprises a polypeptide having at least 60% identity to an amino acid sequence set forth in SEQ ID NO:135, a UGT73C6 polypeptide comprises a polypeptide having at least 60% identity to an amino acid sequence set forth in SEQ ID NO:137, a UGT73E1 polypeptide comprises a polypeptide having at least 50% identity to an amino acid sequence set forth in SEQ ID NO:141, a UGT74D1 polypeptide comprises a polypeptide having at least 50% identity to an amino acid sequence set forth in SEQ ID NO:143, a UGT75B1 polypeptide comprises a polypeptide having at least 50% sequence identity to an amino acid sequence set forth in SEQ ID NO:145, a UGT75L6 polypeptide comprises a polypeptide having at least 60% sequence identity to an amino acid sequence set forth in SEQ ID NO:147, a UGT76E12 polypeptide comprises a polypeptide having at least 60% sequence identity to an amino acid sequence set forth in SEQ ID NO:153, a Olel polypeptide comprises a polypeptide having at least 55% identity to an amino acid sequence set forth in SEQ ID NO:177, a UGTS polypeptide comprises a polypeptide having at least 65% identity to an amino acid sequence set forth in SEQ ID NO:181, a SA Gtase polypeptide comprises a polypeptide having at least 55% identity to an amino acid sequence set forth in SEQ ID NO:183, a UDPG1 polypeptide comprises a polypeptide having at least 50% sequence identity to an amino acid sequence set forth in SEQ ID NO:185, a UN1671 polypeptide comprises a polypeptide having at least 45% identity to an amino acid sequence set forth in SEQ ID NO:201, a UGT74F1 polypeptide comprises a polypeptide having at least 50% sequence identity to an amino acid sequence set forth in SEQ ID NO:203, a UGT75D1 polypeptide comprises a polypeptide having at least 50% sequence identity to an amino acid sequence set forth in SEQ ID NO:205, a UGT84B2 polypeptide comprises a polypeptide having at least 40% sequence identity to an amino acid sequence set forth in SEQ ID NO:207, a UGT74F2-like UGT polypeptide comprises a polypeptide having at least 55% identity to an amino acid sequence set forth in SEQ ID NO:211, a UGT73C7 polypeptide comprises a polypeptide having at least 60% identity to an amino acid sequence set forth in SEQ ID NO:139, a CaUGT3 polypeptide comprises a polypeptide having at least 50% identity to an amino acid sequence set forth in SEQ ID NO:169, a UN32491 polypeptide comprises a polypeptide having at least 50% identity to an amino acid sequence set forth in SEQ ID NO:199, or a CaUGT2 polypeptide comprises a polypeptide having at least 55% identity to an amino acid sequence set forth in SEQ ID NO:209;

and a plant-derived or synthetic steviol glycoside precursor or a plant-derived or synthetic steviol precursor to a reaction mixture;

wherein at least one of the polypeptides is a recombinant polypeptide; and

producing the one or more steviol glycosides and/or glycosylated steviol precursors, or the composition thereof, thereby.

In one aspect of the method disclosed herein, the reaction mixture comprises:
(a) glucose, fructose, sucrose, xylose, rhamnose, uridine diphosphate (UDP)-glucose, UDP-rhamnose, UDP-xylose, and/or N-acetyl-glucosamine; and/or
(b) reaction buffer and/or salts.

In one aspect of the method disclosed herein, the one or more steviol glycosides and/or glycosylated steviol precursors are, or the composition thereof comprises, 13-SMG, 19-SMG, steviol-1,2-bioside, steviol-1,3-bioside, 1,2-stevioside, 1,3-stevioside, rubusoside, RebA, RebB, RebC, RebD, RebE, RebF, RebM, RebQ, RebI, dulcoside A, a mono-glycosylated ent-kaurenoic acid, a di-glycosylated ent-kaurenoic acid, a tri-glycosylated ent-kaurenoic acid, a mono-glycosylated ent-kaurenols, a di-glycosylated ent-kaurenol, a tri-glycosylated ent-kaurenol, a tri-glycosylated steviol glycoside, a tetra-glycosylated steviol glycoside, a penta-glycosylated steviol glycoside, a hexa-glycosylated steviol glycoside, a hepta-glycosylated steviol glycoside, and/or an isomer thereof.

In one aspect of the method disclosed herein, the mono-glycosylated ent-kaurenoic acid comprises KA1.58 of Table 1 and/or the penta-glycosylated steviol comprises Compound 5.24 of Table 1.

The invention also provides a cell culture, comprising the recombinant host cell disclosed herein, the cell culture further comprising:
(a) one or more steviol glycosides and/or glycosylated steviol precursors, or the composition thereof produced by the recombinant host cell,
(b) glucose, fructose, sucrose, xylose, rhamnose, UDP-glucose, UDP-rhamnose, UDP-xylose, and/or N-acetyl-glucosamine; and
(c) supplemental nutrients comprising trace metals, vitamins, salts, yeast nitrogen base (YNB), and/or amino acids;

wherein the one or more steviol glycosides and/or glycosylated steviol precursors, or the composition thereof is present at a concentration of at least 1 mg/liter of the cell culture;

wherein the cell culture is enriched for the one or more steviol glycosides and/or glycosides of a steviol precursor, or the composition thereof relative to a steviol glycoside composition from a Stevia plant and has a reduced level of Stevia plant-derived components relative to a plant-derived Stevia extract.

The invention also provides a cell lysate from the recombinant host cell disclosed herein grown in the cell culture, comprising:
(a) one or more steviol glycosides and/or glycosylated steviol precursors, or the composition thereof produced by the recombinant host cell;
(b) glucose, fructose, sucrose, xylose, rhamnose, UDP-glucose, UDP-rhamnose, UDP-xylose, and/or N-acetyl-glucosamine; and/or
(c) supplemental nutrients comprising trace metals, vitamins, salts, yeast nitrogen base, YNB, and/or amino acids;

wherein the one or more steviol glycosides and/or glycosylated steviol precursors, or the composition thereof produced by the recombinant host cell is present at a concentration of at least 1 mg/liter of the cell culture.

The invention also provides a reaction mixture, comprising:
(a) a UGT85C2 polypeptide having at least 55% identity to an amino acid sequence set forth in SEQ ID NO:7;
(b) a UGT76G1 polypeptide having at least 50% identity to an amino acid sequence set forth in SEQ ID NO:9;
(c) a UGT74G1 polypeptide having at least 55% identity to an amino acid sequence set forth in SEQ ID NO:4;
(d) a UGT91D2 functional homolog polypeptide comprising a UGT91D2e polypeptide having 90% or greater identity to an amino acid sequence set forth in SEQ ID NO:11 or a UGT91D2e-b polypeptide having 90% or greater identity to an amino acid sequence set forth in SEQ ID NO:13;
(e) a EUGT11 polypeptide having at least 65% identity to an amino acid sequence set forth in SEQ ID NO:16; and/or
(f) a UGT73C1 polypeptide comprises a polypeptide having at least 60% identity to an amino acid sequence set forth in SEQ ID NO:127, a UGT73C3 polypeptide comprises a polypeptide having at least 60% identity to an amino acid sequence set forth in SEQ ID NO:133, a UGT73C5 polypeptide comprises a polypeptide having at least 60% identity to an amino acid sequence set forth in SEQ ID NO:135, a UGT73C6 polypeptide comprises a polypeptide having at least 60% identity to an amino acid sequence set forth in SEQ ID NO:137, a UGT73E1 polypeptide comprises a polypeptide having at least 50% identity to an amino acid sequence set forth in SEQ ID NO:141, a UGT75B1 polypeptide comprises a polypeptide having at least 50% sequence identity to an amino acid sequence set forth in SEQ ID NO:145, a UGT75L6 polypeptide comprises a polypeptide having at least 60% sequence identity to an amino acid sequence set forth in SEQ ID NO:147, a UGT76E12 polypeptide comprises a polypeptide having at least 60% sequence identity to an amino acid sequence set forth in SEQ ID NO:153, a Olel polypeptide comprises a polypeptide having at least 55% identity to an amino acid sequence set forth in SEQ ID NO:177, a UGTS polypeptide comprises a polypeptide having at least 65% identity to an amino acid sequence set forth in SEQ ID NO:181, a SA Gtase polypeptide comprises a polypeptide having at least 55% identity to an amino acid sequence set forth in SEQ ID NO:183, a UDPG1 polypeptide comprises a polypeptide having at least 50% sequence identity to an amino acid sequence set forth in SEQ ID NO:185, a UN1671 polypeptide comprises a polypeptide having at least 45% identity to an amino acid sequence set forth in SEQ ID NO:201, a UGT74F1 polypeptide comprises a polypeptide having at least 50% sequence identity to an amino acid sequence set forth in SEQ ID NO:203, a UGT75D1 polypeptide comprises a polypeptide having at least 50% sequence identity to an amino acid sequence set forth in SEQ ID NO:205, a UGT84B2 polypeptide comprises a polypeptide having at least 40% sequence identity to an amino acid sequence set forth in SEQ ID NO:207, a UGT74F2-like UGT polypeptide comprises a polypeptide having at least 55% identity to an amino acid sequence set forth in SEQ ID NO:211, a UGT73C7 polypeptide comprises a polypeptide having at least 60% identity to an amino acid sequence set forth in SEQ ID NO:139, a CaUGT3 polypeptide comprises a polypeptide having at least 50% identity to an amino acid sequence set forth in SEQ ID NO:169, or a UN32491 polypeptide comprises a polypeptide having at least 50% identity to an amino acid sequence set forth in SEQ ID NO:199;
   and further comprising:
(g) one or more steviol glycosides and/or glycosylated steviol precursors, or a composition thereof;
(h) glucose, fructose, sucrose, xylose, rhamnose, uridine diphosphate (UDP)-glucose, UDP-rhamnose, UDP-xylose, and/or N-acetyl-glucosamine; and/or
(i) reaction buffer and/or salts.

The invention also provides a composition of one or more steviol glycosides and/or glycosylated steviol precursors produced by the recombinant host cell disclosed herein; wherein the one or more steviol glycosides and/or glycosylated steviol precursors produced by the recombinant host cell are present in relative amounts that are different from a steviol glycoside composition from a Stevia plant and have a reduced level of Stevia plant-derived components relative to a plant-derived Stevia extract.

The invention also provides a composition of one or more steviol glycosides and/or glycosylated steviol precursors produced by the method disclosed herein; wherein the one or more steviol glycosides and/or glycosylated steviol precursors produced by the recombinant host cell are present in relative amounts that are different from a steviol glycoside composition from a Stevia plant and have a reduced level of Stevia plant-derived components relative to a plant-derived Stevia extract.

The invention also provides a sweetener composition, comprising one or more steviol glycosides and/or glycosylated steviol precursors produced by the recombinant host cell and/or the method disclosed herein.

The invention also provides a food product, comprising the sweetener composition disclosed herein.

The invention also provides a beverage or a beverage concentrate, comprising the sweetener composition disclosed herein.

The invention also provides an isolated nucleic acid molecule encoding a polypeptide capable of glycosylating steviol or a steviol glycoside at its C-19 carboxyl position or a catalytically active portion thereof, wherein the encoded polypeptide capable of glycosylating steviol or a steviol glycoside at its C-19 carboxyl position or the catalytically active portion thereof has at least 60% sequence identity to the amino acid sequence set forth in SEQ ID NO:127, at least 60% sequence identity to the amino acid sequence set forth in SEQ ID NO:133, at least 60% sequence identity to the amino acid sequence set forth in SEQ ID NO:135, at least 60% sequence identity to the amino acid sequence set forth in SEQ ID NO:137, at least 50% sequence identity to the amino acid sequence set forth in SEQ ID NO:141, at least 50% sequence identity to the amino acid sequence set forth in SEQ ID NO:145, at least 60% sequence identity to the amino acid sequence set forth in SEQ ID NO:147, at least 55% sequence identity to the amino acid sequence set forth in SEQ ID NO:177, at least 65% sequence identity to the amino acid sequence set forth in SEQ ID NO:181, at least 55% sequence identity to the amino acid sequence set forth in SEQ ID NO:183, at least 50% sequence identity to the amino acid sequence set forth in SEQ ID NO:185, at least 45% sequence identity to the amino acid sequence set forth in SEQ ID NO:201, at least 50% sequence identity to the amino acid sequence set forth in SEQ ID NO:203, at least 40% sequence identity to the amino acid sequence set forth in SEQ ID NO:207, or at least 55% sequence identity to the amino acid sequence set forth in SEQ ID NO:211.

The invention also provides an isolated nucleic acid molecule encoding a polypeptide capable of glycosylating steviol or a steviol glycoside at its C-13 hydroxyl position or a catalytically active portion thereof, wherein the encoded polypeptide capable of glycosylating steviol or a steviol glycoside at its C-13 hydroxyl position or the catalytically active portion thereof has at least 60% sequence identity to the amino acid sequence set forth in SEQ ID NO:127, at least 60% sequence identity to the amino acid sequence set forth in SEQ ID NO:133, at least 60% sequence identity to the amino acid sequence set forth in SEQ ID NO:135, at least 60% sequence identity to the amino acid sequence set forth in SEQ ID NO:137, at least 60% sequence identity to the amino acid sequence set forth in SEQ ID NO:139, at least 50% sequence identity to the amino acid sequence set forth in SEQ ID NO:141, or at least 60% sequence identity to the amino acid sequence set forth in SEQ ID NO:153.

The invention also provides an isolated nucleic acid molecule encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside or a catalytically active portion thereof, wherein the encoded polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside or the catalytically active portion thereof has at least 60% sequence identity to the amino acid sequence set forth in SEQ ID NO:137, at least 50% sequence identity to the amino acid sequence set forth in SEQ ID NO:169, at least 50% sequence identity to the amino acid sequence set forth in SEQ ID NO:199, or at least 45% sequence identity to the amino acid sequence set forth in SEQ ID NO:201.

The invention also provides an isolated nucleic acid molecule encoding a polypeptide capable of glycosylating a steviol precursor at its C-19 carboxyl or C-19 hydroxyl position or a catalytically active portion thereof, wherein the encoded polypeptide capable of glycosylating a steviol precursor at its C-19 carboxyl or C-19 hydroxyl position or the catalytically active portion thereof has at least 60% sequence identity to the amino acid sequence set forth in SEQ ID NO:127, at least 60% sequence identity to the amino acid sequence set forth in SEQ ID NO:133, at least 60% sequence identity to the amino acid sequence set forth in SEQ ID NO:135, at least 60% sequence identity to the amino acid sequence set forth in SEQ ID NO:137, at least 50% sequence identity to the amino acid sequence set forth in SEQ ID NO:141, at least 50% sequence identity to the amino acid sequence set forth in SEQ ID NO:145, at least 60% sequence identity to the amino acid sequence set forth in SEQ ID NO:147, at least 60% sequence identity to the amino acid sequence set forth in SEQ ID NO:153, at least 55% sequence identity to the amino acid sequence set forth in SEQ ID NO:177, at least 65% sequence identity to the amino acid sequence set forth in SEQ ID NO:181, at least 55% sequence identity to the amino acid sequence set forth in SEQ ID NO:183, at least 50% sequence identity to the amino acid sequence set forth in SEQ ID NO:185, at least 50% sequence identity to the amino acid sequence set forth in SEQ ID NO:203, at least 50% sequence identity to the amino acid sequence set forth in SEQ ID NO:205, at least 40% sequence identity to the amino acid sequence set forth in SEQ ID NO:207, or at least 55% sequence identity to the amino acid sequence set forth in SEQ ID NO:211.

In one aspect of the isolated nucleic acids disclosed herein, the nucleic acid is cDNA.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows representative primary steviol glycoside glycosylation reactions catalyzed by suitable uridine 5′-diphospho (UDP) glycosyl transferases (UGT) enzymes and chemical structures for several of the compounds found in Stevia extracts.

FIG. 2 shows the biochemical pathway for producing steviol from geranylgeranyl diphosphate using geranylgeranyl diphosphate synthase (GGPPS), ent-copalyl diphosphate synthase (CDPS), ent-kaurene synthase (KS), ent-kaurene oxidase (KO), and ent-kaurenoic acid hydroxylase (KAH) polypeptides.

FIG. 3 shows the structures of steviol+6Glc (isomer 1) and steviol+7Glc (isomer 2).

FIG. 4 shows the structures of steviol+4Glc (#26) and ent-kaurenoic Acid+3Glc (isomer 1).

FIG. 5 shows the structures ent-kaurenoic acid+3Glc (isomer 2) and ent-kaurenol+3Glc (isomer 1).

FIGS. 6A, 6B, and 6C show a ¹H NMR spectrum and ¹H and ¹³C NMR chemical shifts (in ppm) for ent-kaurenoic acid+3Glc (isomer 1). FIGS. 6D, 6E, and 6F show a ¹H NMR spectrum and ¹H and ¹³C NMR chemical shifts (in ppm) for ent-kaurenoic acid+3Glc (isomer 2). FIGS. 6G, 6H, and 6I show a ¹H NMR spectrum and ¹H and ¹³C NMR chemical shifts (in ppm) for ent-kaurenol+3Glc (isomer 1). FIGS. 6J, 6K, 6L, and 6M show a ¹H NMR spectrum and ¹H and ¹³C NMR chemical shifts (in ppm) for steviol+6Glc (isomer 1). FIGS. 6N, 6O, 6P, and 6Q show a ¹H NMR spectrum and ¹H and ¹³C NMR chemical shifts (in ppm) for steviol+7Glc (isomer 2). FIGS. 6R, 6S, 6T, and 6U show a ¹H NMR spectrum and ¹H and ¹³C NMR chemical shifts (in ppm) for steviol+4Glc (#26).

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

As used herein, the terms “microorganism,” “microorganism host,” and “microorganism host cell” can be used interchangeably. As used herein, the terms “recombinant host” and “recombinant host cell” can be used interchangeably. The person of ordinary skill in the art will appreciate that the terms “microorganism,” microorganism host,” and “microorganism host cell,” when used to describe a cell comprising a recombinant gene, may be taken to mean “recombinant host” or “recombinant host cell.” As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in S. cerevisiae.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to S. cerevisiae, including, but not limited to S. cerevisiae strain S288C. In some embodiments, an endogenous yeast gene is overexpressed. As used herein, the term “overexpress” is used to refer to the expression of a gene in an organism at levels higher than the level of gene expression in a wild type organism. See, e.g., Prelich, 2012, Genetics 190:841-54. In some embodiments, an endogenous yeast gene, for example ADH, is deleted. See, e.g., Giaever & Nislow, 2014, Genetics 197(2):451-65. As used herein, the terms “deletion,” “deleted,” “knockout,” and “knocked out” can be used interchangabley to refer to an endogenous gene that has been manipulated to no longer be expressed in an organism, including, but not limited to, S. cerevisiae.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see, e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

As used herein, the term “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

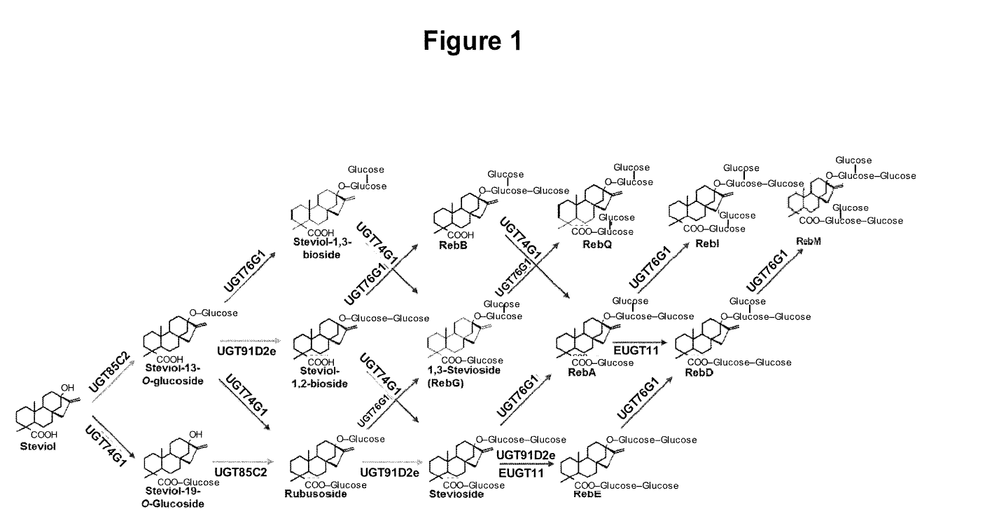

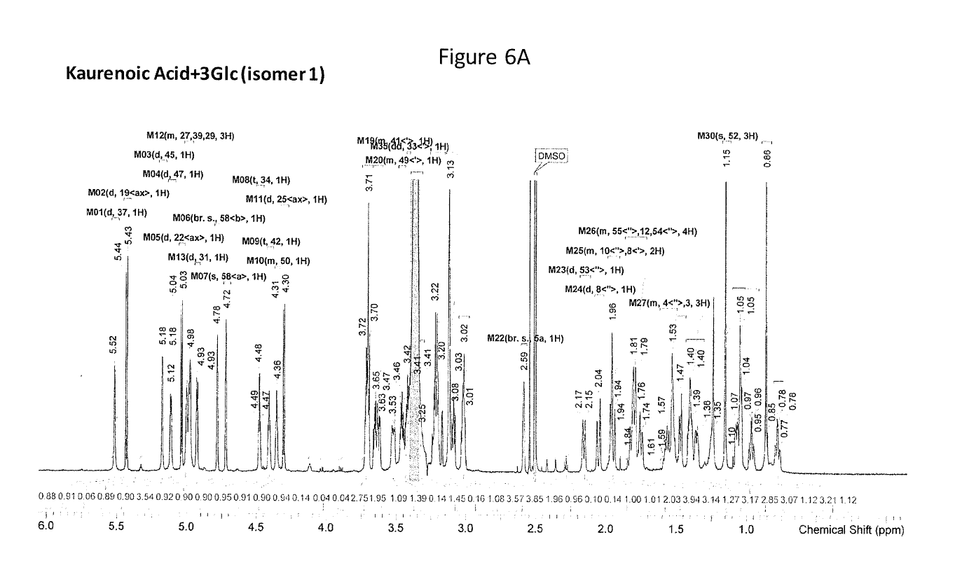

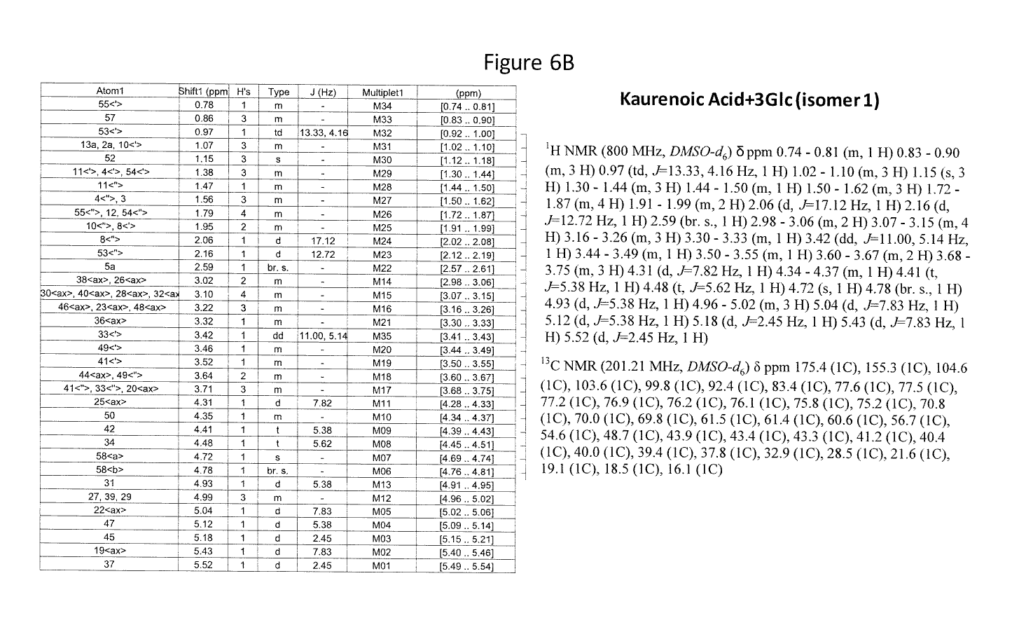

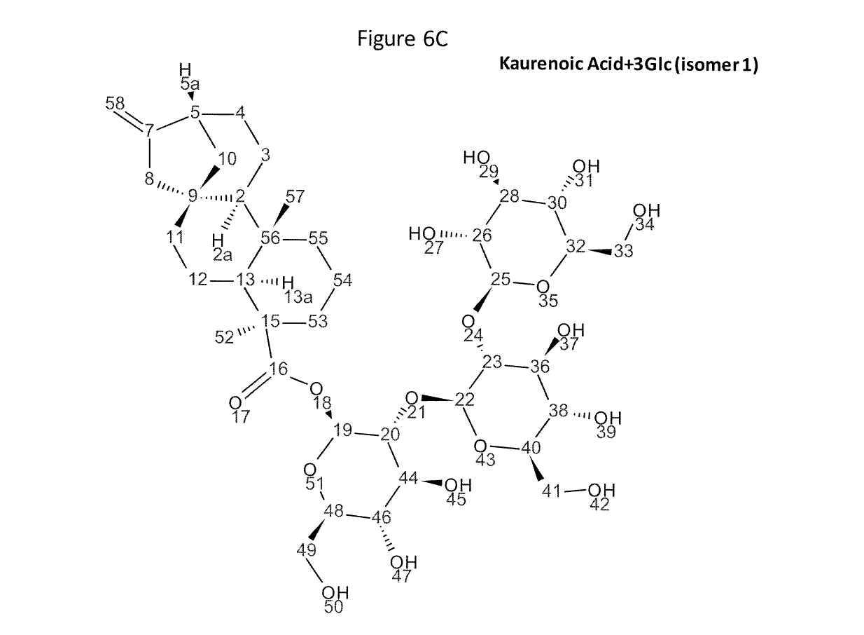

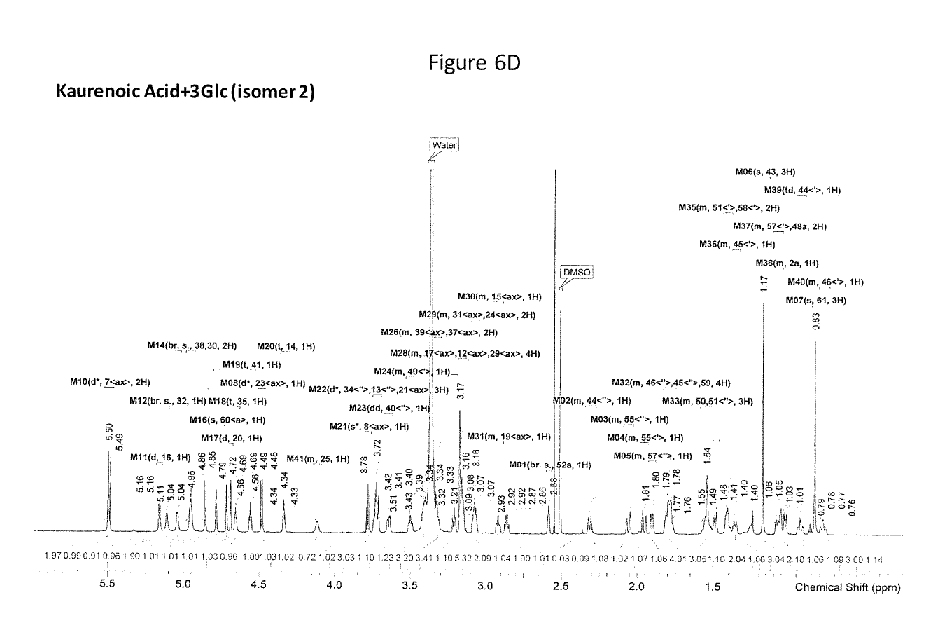

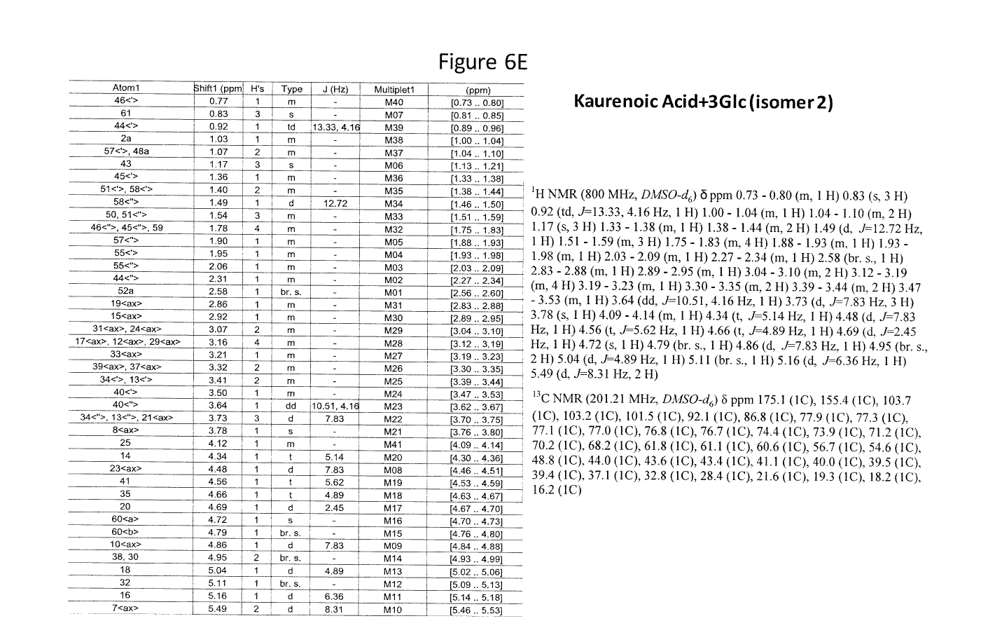

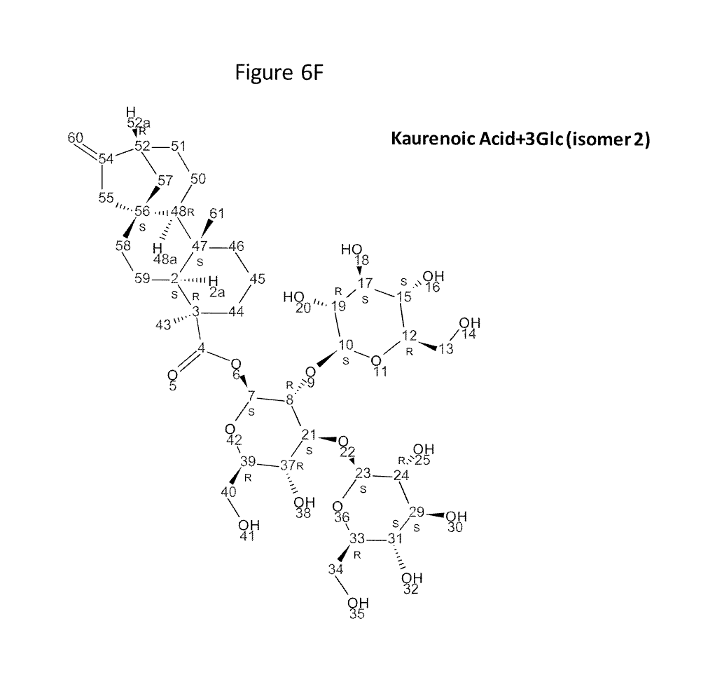

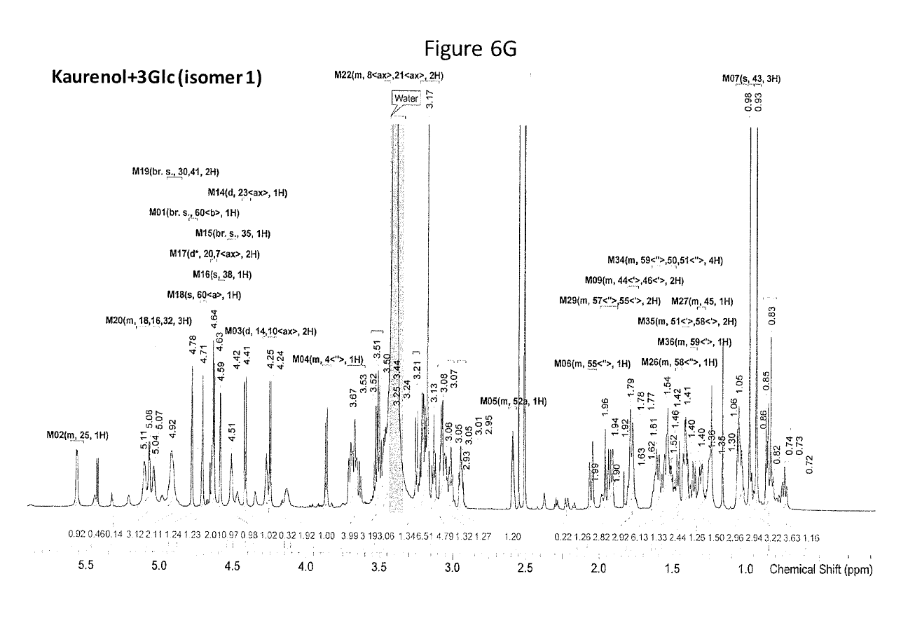

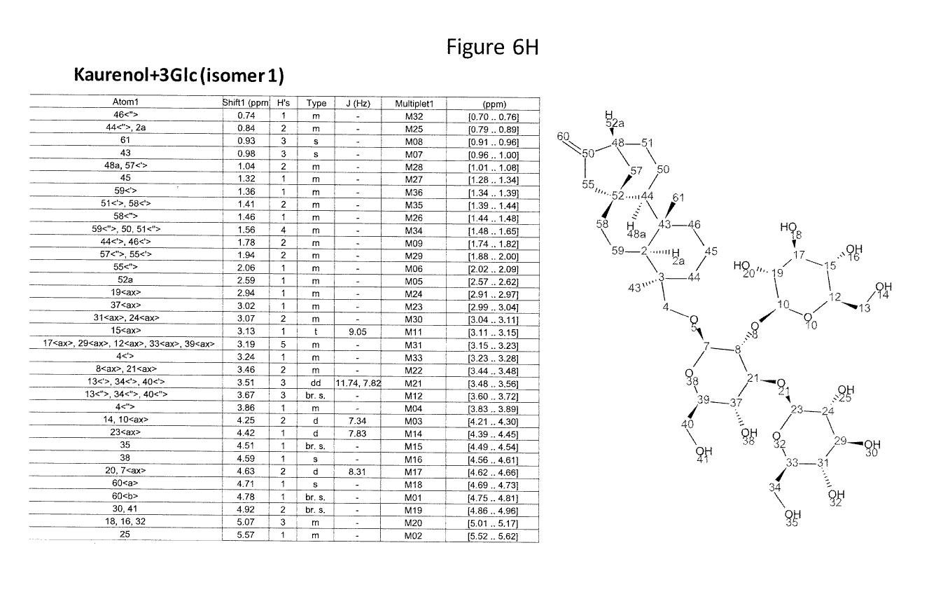

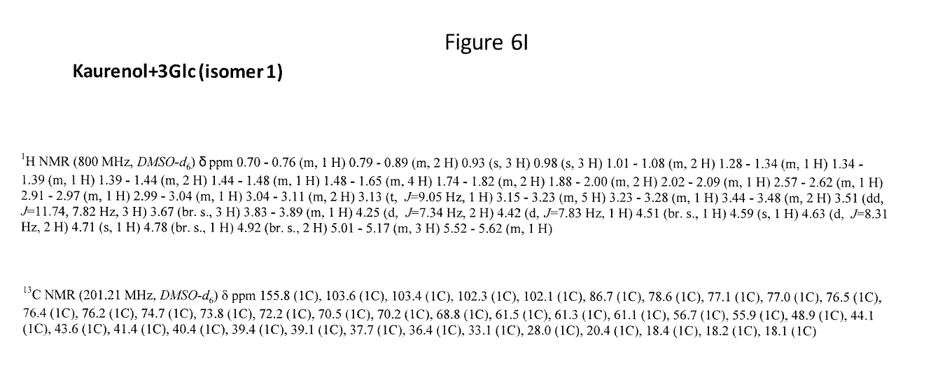

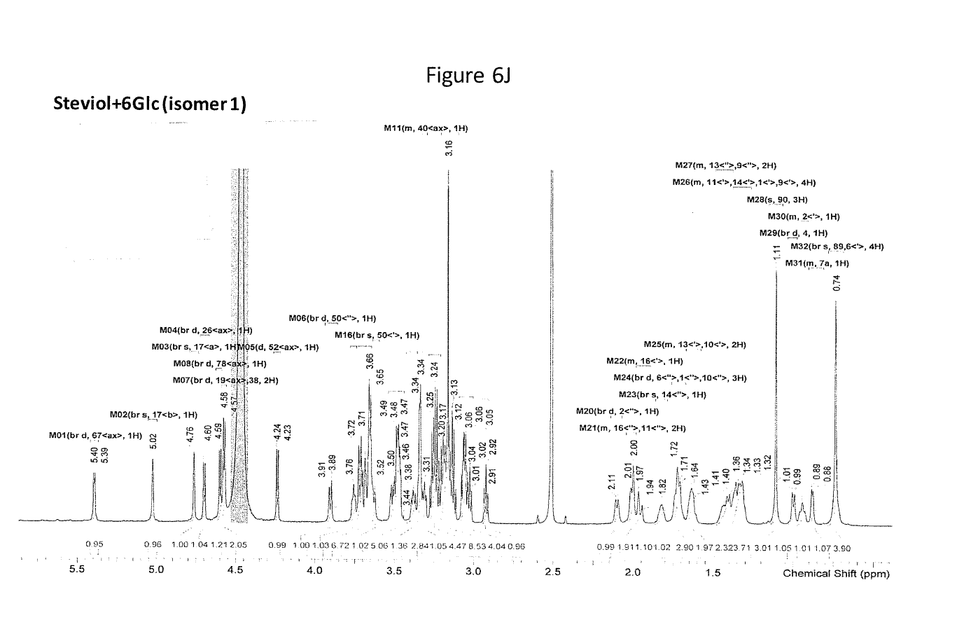

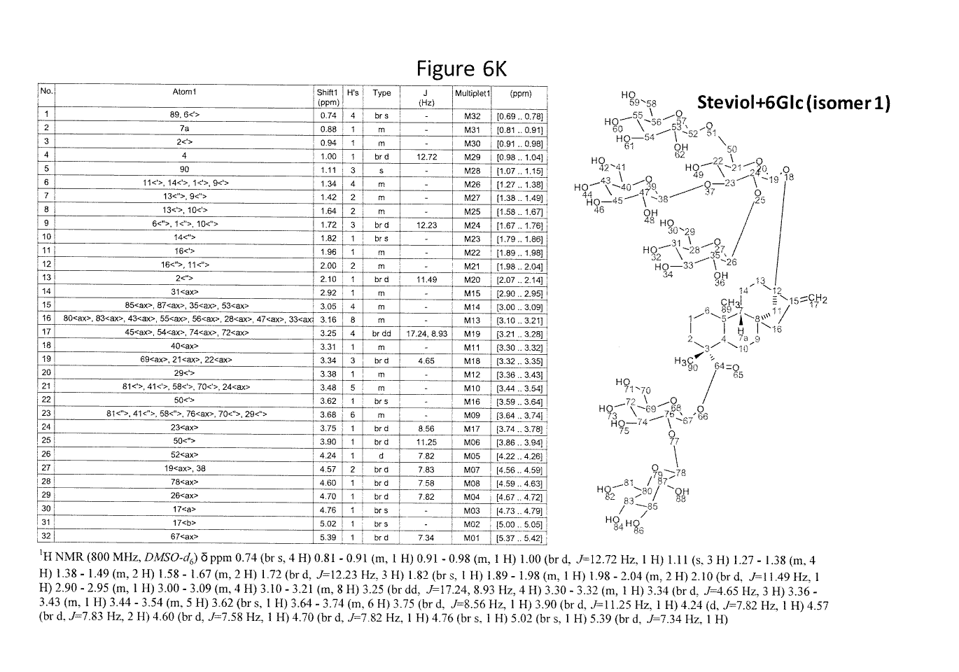

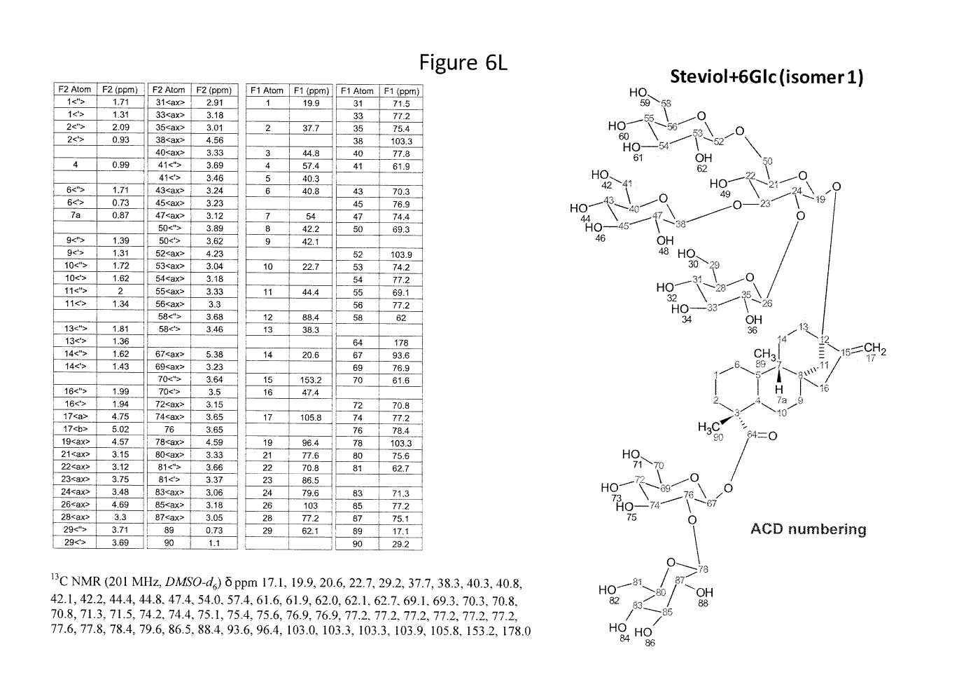

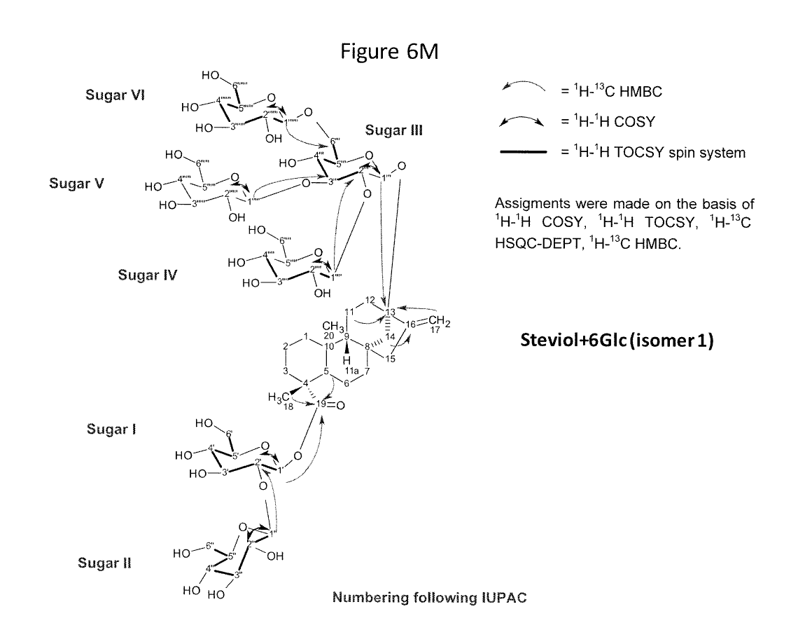

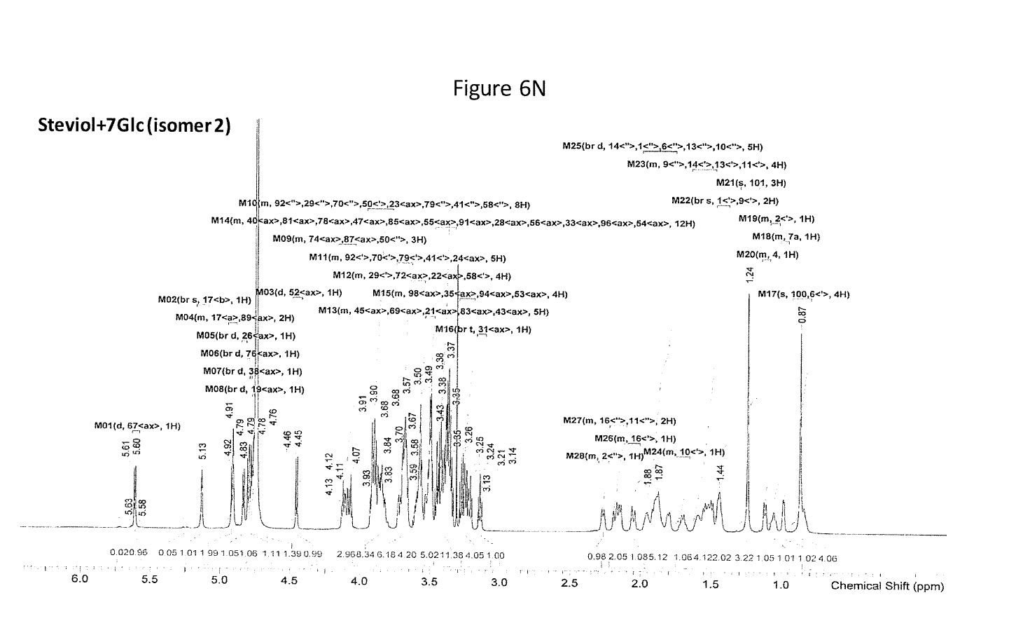

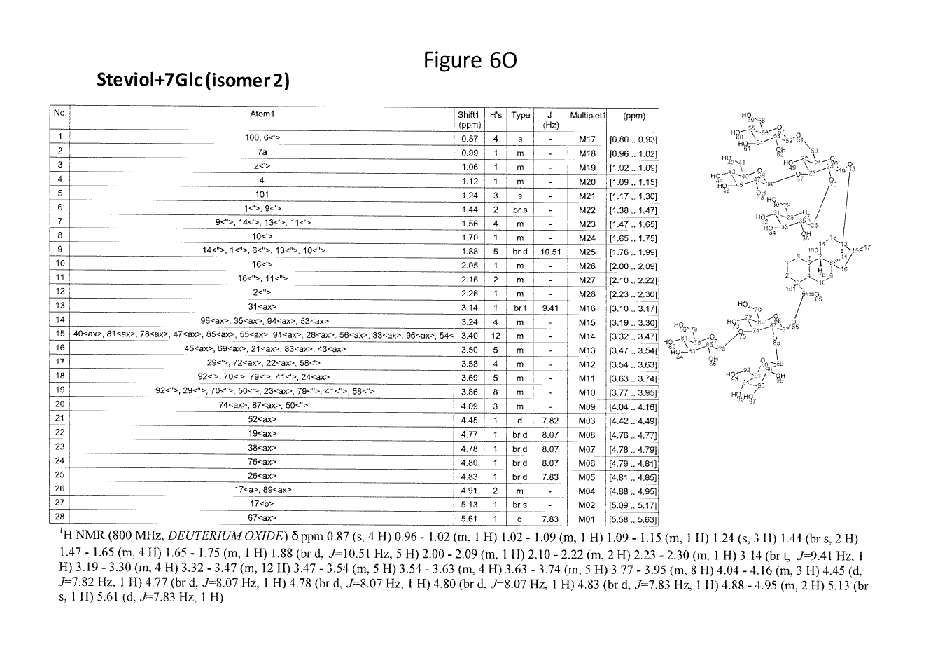

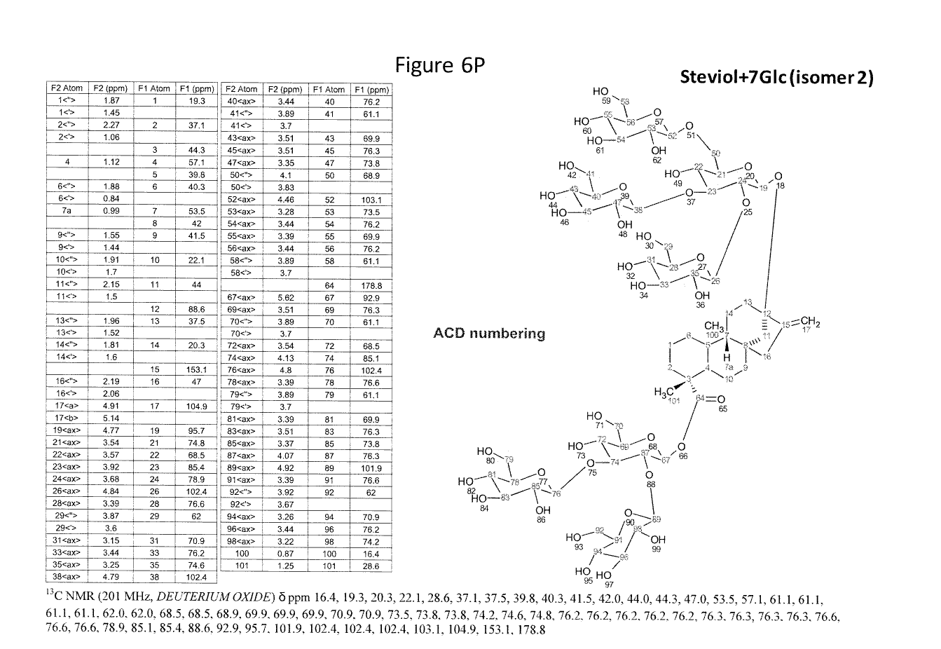

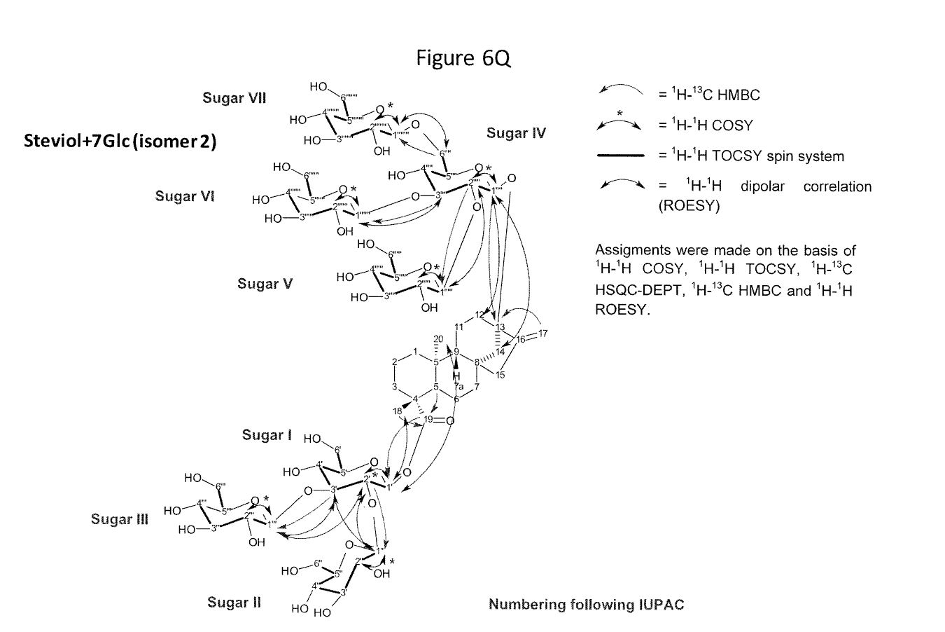

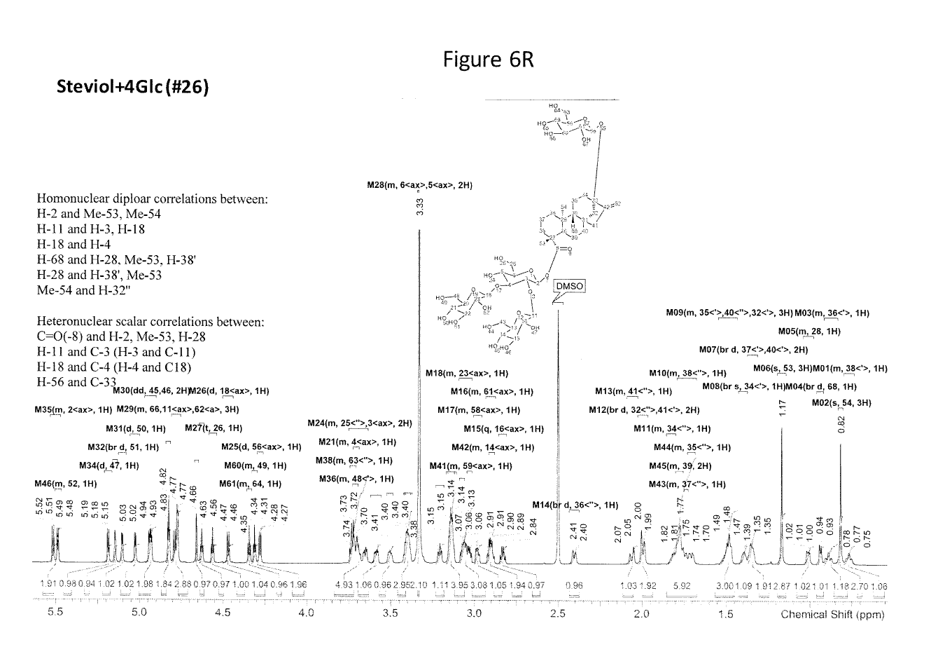

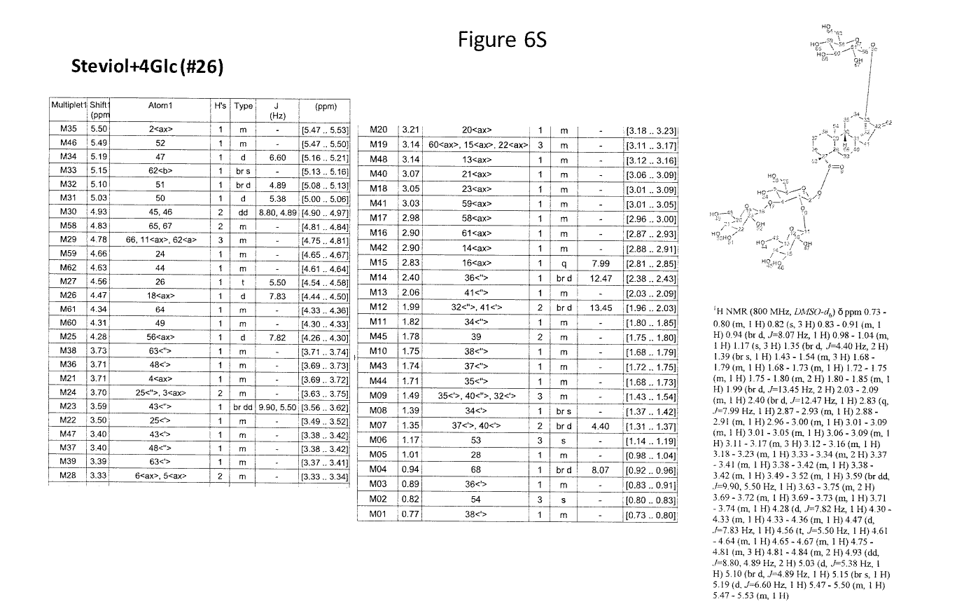

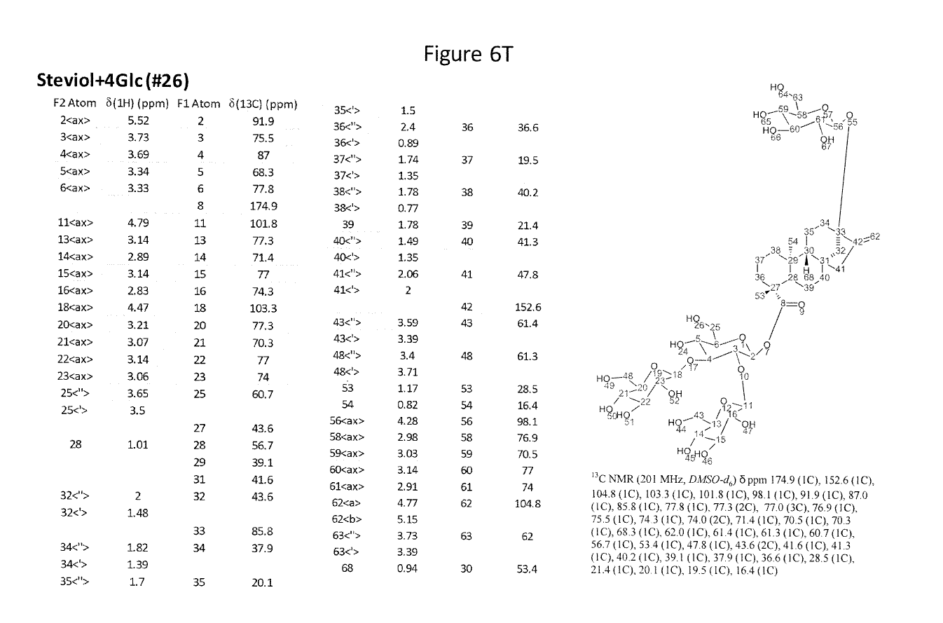

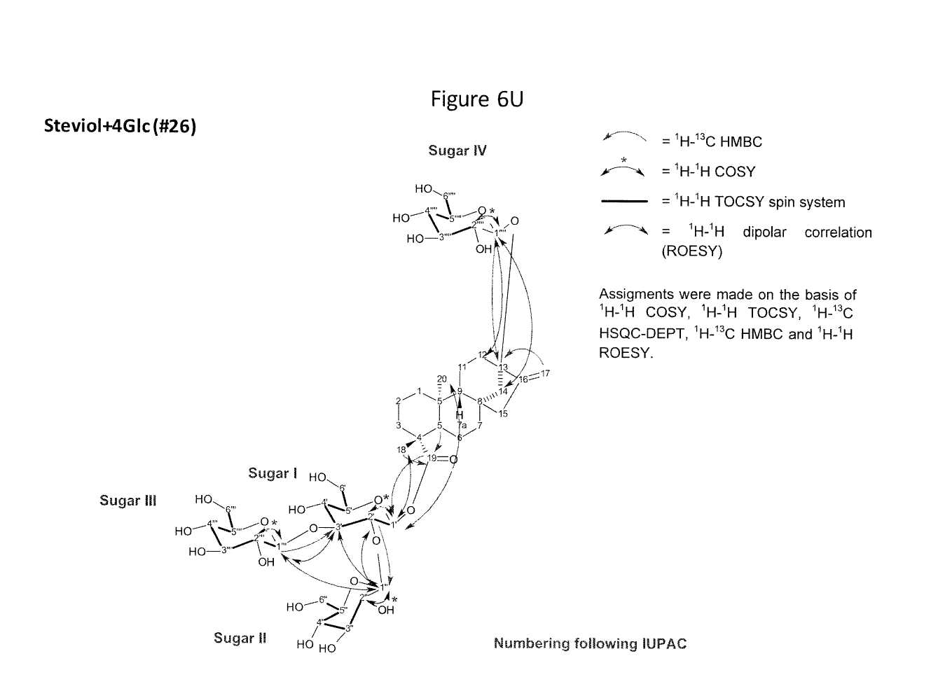

As used herein, the term “steviol glycoside” refers to rebaudioside A (RebA) (CAS #58543-16-1), rebaudioside B (RebB) (CAS #58543-17-2), rebaudioside C (RebC) (CAS #63550-99-2), rebaudioside D (RebD) (CAS #63279-13-0), rebaudioside E (RebE) (CAS #63279-14-1), rebaudioside F (RebF) (CAS #438045-89-7), rebaudioside M (RebM) (CAS #1220616-44-3), rubusoside (CAS #63849-39-4), Dulcoside A (CAS #64432-06-0), rebaudioside I (RebI) (MassBank Record: FU000332), rebaudioside Q (RebQ), 1,2-stevioside (CAS #57817-89-7), 1,3-stevioside (RebG), steviol-1,2-bioside (MassBank Record: FU000299), steviol-1,3-bioside, steviol-13-O-glucoside (13-SMG), steviol-19-O-glucoside (19-SMG), a tri-glucosylated steviol glycoside, a tetra-glycosylated steviol glycoside, a penta-glucosylated steviol glycoside, a hexa-glucosylated steviol glycoside, a hepta-glucosylated steviol glycoside, and isomers thereof. See FIG. 1; see also, Steviol Glycosides Chemical and Technical Assessment 69th JECFA, 2007, prepared by Harriet Wallin, Food Agric. Org. Nuclear magnetic resonance (NMR) spectra for steviol glycoside isomers disclosed herein can be found in FIG. 6.

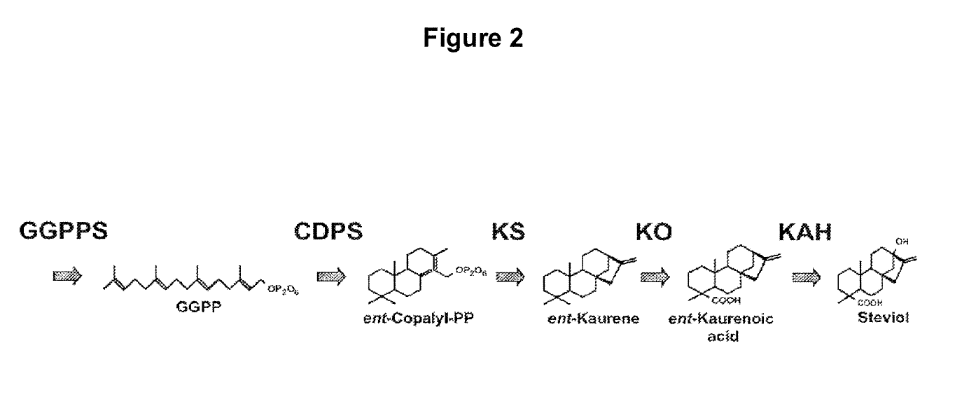

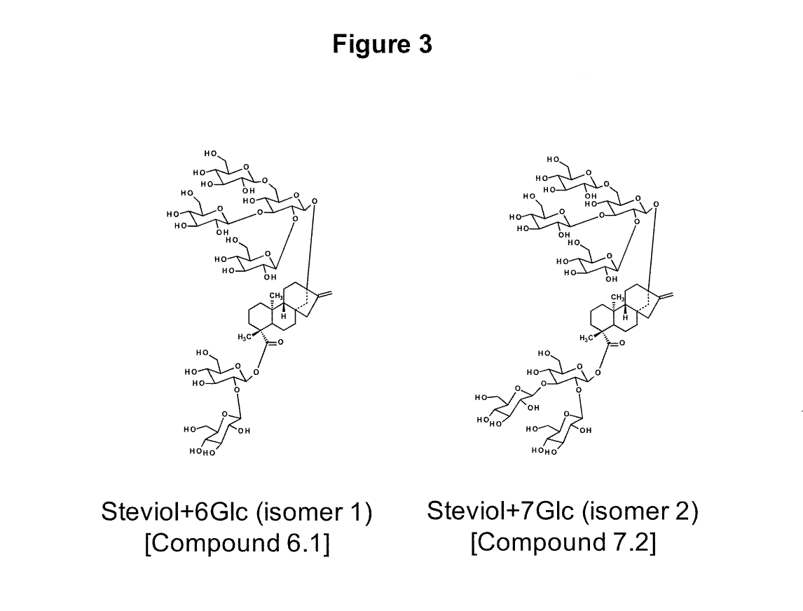

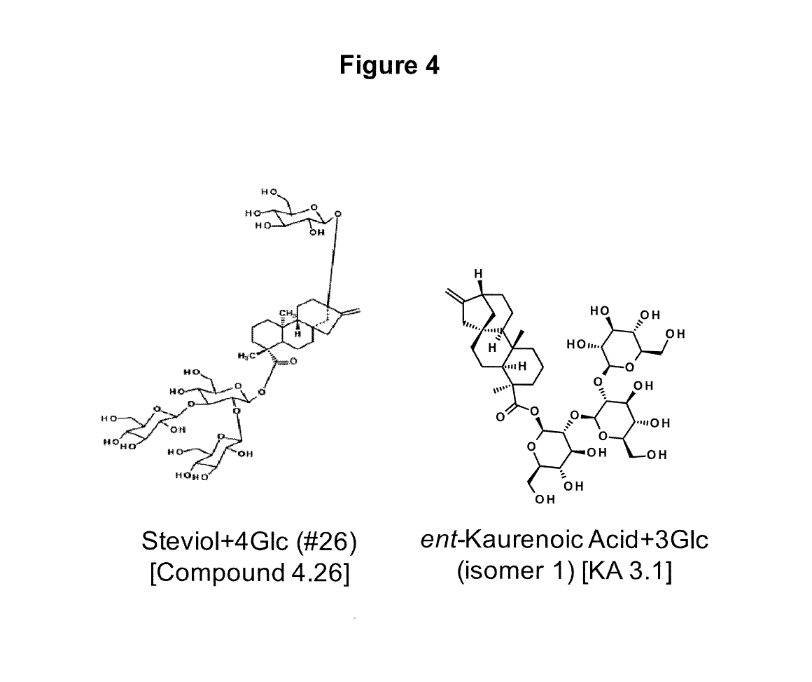

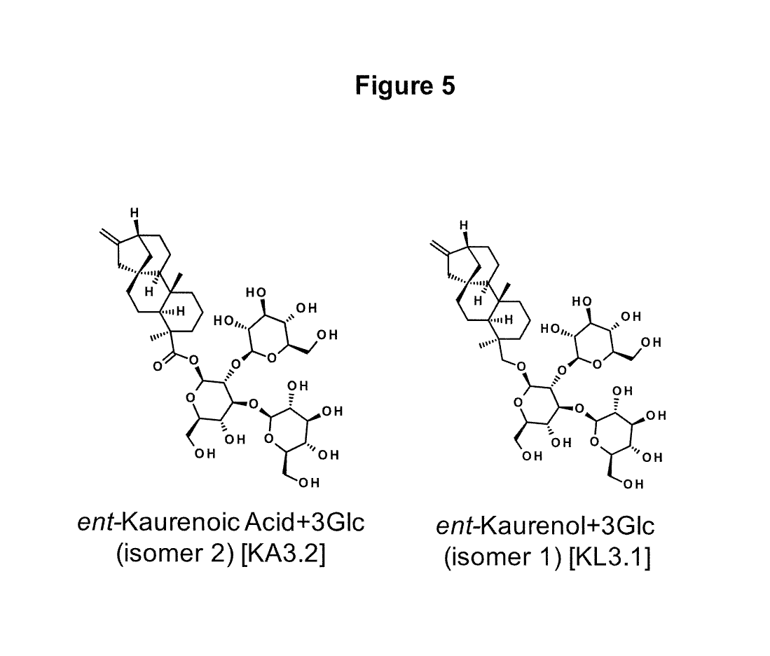

As used herein, the terms “steviol glycoside precursor” and “steviol glycoside precursor compound” are used to refer to intermediate compounds in the steviol glycoside biosynthetic pathway. Steviol glycoside precursors include, but are not limited to, geranylgeranyl diphosphate (GGPP), ent-copalyl-diphosphate, ent-kaurene, ent-kaurenol, ent-kaurenal, ent-kaurenoic acid, and steviol. See FIG. 2. Also as used herein, the terms “steviol precursor” and “steviol precursor compound” are used to refer to intermediate compounds in the steviol biosynthetic pathway (i.e., compounds from which steviol may ultimately be synthesized). Steviol precursors include, but are not limited to, geranylgeranyl diphosphate (GGPP), ent-copalyl-diphosphate, ent-kaurene, ent-kaurenol, ent-kaurenal, and ent-kaurenoic acid. In some embodiments, steviol precurors can be glycosylated, e.g., tri-glycosylated ent-kaurenoic acid (ent-kaurenoic acid+3Glc), di-glycosylated ent-kaurenoic acid, mono-glycosylated ent-kaurenoic acid, tri-glycosylated ent-kaurenol, di-glycosylated ent-kaurenol (ent-kaurenol+2Glc), or mono-glycosylated ent-kaurenol (ent-kaurenol+1Glc). The person of ordinary skill in the art will appreciate that steviol precursors may be steviol glycoside precursors. In some embodiments, steviol glycoside precursors are themselves steviol glycoside compounds. For example, 19-SMG, rubusoside, stevioside, and RebE are steviol glycoside precursors of RebM. See FIG. 1.

As used herein, the term “contact” is used to refer to any physical interaction between two objects. For example, the term “contact” may refer to the interaction between an an enzyme and a substrate. In another example, the term “contact” may refer to the interaction between a liquid (e.g., a supernatant) and an adsorbent resin.

Steviol glycosides, steviol glycoside precursors, and/or glycosides of steviol precursors can be produced in vivo (i.e., in a recombinant host), in vitro (i.e., enzymatically), or by whole cell bioconversion. As used herein, the terms “produce” and “accumulate” can be used interchangeably to describe synthesis of steviol glycosides, glycosides of steviol precursors, and steviol glycoside precursors in vivo, in vitro, or by whole cell bioconversion.

Recombinant steviol glycoside-producing Saccharomyces cerevisiae (S. cerevisiae) strains are described in WO 2011/153378, WO 2013/022989, WO 2014/122227, and WO 2014/122328. Methods of producing steviol glycosides in recombinant hosts, by whole cell bio-conversion, and in vitro are also described in WO 2011/153378, WO 2013/022989, WO 2014/122227, and WO 2014/122328.

As used herein, the terms “culture broth,” “culture medium,” and “growth medium” can be used interchangeably to refer to a liquid or solid that supports growth of a cell. A culture broth can comprise glucose, fructose, sucrose, trace metals, vitamins, salts, yeast nitrogen base (YNB), and/or amino acids. The trace metals can be divalent cations, including, but not limited to, Mn²⁺ and/or Mg²⁺. In some embodiments, Mn²⁺ can be in the form of MnCl₂ dihydrate and range from approximately 0.01 g/L to 100 g/L. In some embodiments, Mg²⁺ can be in the form of MgSO₄ heptahydrate and range from approximately 0.01 g/L to 100 g/L. For example, a culture broth can comprise i) approximately 0.02-0.03 g/L MnCl₂ dihydrate and approximately 0.5-3.8 g/L MgSO₄ heptahydrate, ii) approximately 0.03-0.06 g/L MnCl₂ dihydrate and approximately 0.5-3.8 g/L MgSO₄ heptahydrate, and/or iii) approximately 0.03-0.17 g/L MnCl₂ dihydrate and approximately 0.5-7.3 g/L MgSO₄ heptahydrate. Additionally, a culture broth can comprise one or more steviol glycosides produced by a recombinant host, as described herein.

In some embodiments, a recombinant host comprising a gene encoding a polypeptide capable of synthesizing geranylgeranyl pyrophosphate (GGPP) from farnesyl diphosphate (FPP) and isopentenyl diphosphate (IPP) (e.g., geranylgeranyl diphosphate synthase (GGPPS)); a gene encoding a polypeptide capable of synthesizing ent-copalyl diphosphate from GGPP (e.g., ent-copalyl diphosphate synthase (CDPS)); a gene encoding a polypeptide capable of synthesizing ent-kaurene from ent-copalyl diphosphate (e.g., kaurene synthase (KS)); a gene encoding a polypeptide capable of synthesizing ent-kaurenoic acid, ent-kaurenol, and/or ent-kaurenol from ent-kaurene (e.g., kaurene oxidase (KO)); a gene encoding a polypeptide capable of reducing cytochrome P450 complex (e.g., cytochrome P450 reductase (CPR) or P450 oxidoreductase (POR); for example, but not limited to a polypeptide capable of electron transfer from NADPH to cytochrome P450 complex during conversion of NADPH to NADP⁺, which is utilized as a cofactor for terpenoid biosynthesis); a gene encoding a polypeptide capable of synthesizing steviol from ent-kaurenoic acid (e.g., steviol synthase (KAH)); and/or a gene encoding a bifunctional polypeptide capable of synthesizing ent-copalyl diphosphate from GGPP and synthesizing ent-kaurene from ent-copalyl diphosphate (e.g., an ent-copalyl diphosphate synthase (CDPS)—ent-kaurene synthase (KS) polypeptide) can produce steviol in vivo. See, e.g., FIG. 1. The skilled worker will appreciate that one or more of these genes can be endogenous to the host provided that at least one (and in some embodiments, all) of these genes is a recombinant gene introduced into the recombinant host.

In some embodiments, a recombinant host comprising a gene encoding a polypeptide capable of glycosylating steviol or a steviol glycoside at its C-13 hydroxyl position (e.g., a UGT85C2 polypeptide); a gene encoding a polypeptide capable of beta 1,3 glycosylation of the C3′ of the 13-O-glucose, 19-O-glucose, or both 13-O-glucose and 19-O-glucose of a steviol glycoside (e.g., a UGT76G1 polypeptide); a gene encoding a polypeptide capable of glycosylating steviol or a steviol glycoside at its C-19 carboxyl position (e.g., a UGT74G1 polypeptide); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a UGT91D2 or EUGT11 polypeptide) can produce a steviol glycoside in vivo. The skilled worker will appreciate that one or more of these genes can be endogenous to the host provided that at least one (and in some embodiments, all) of these genes is a recombinant gene introduced into the recombinant host.

In some embodiments, steviol glycosides, glycosides of steviol precursors, and/or steviol glycoside precursors are produced in vivo through expression of one or more enzymes involved in the steviol glycoside biosynthetic pathway in a recombinant host. For example, a recombinant host comprising a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a bifunctional polypeptide capable of synthesizing ent-copalyl diphosphate from GGPP and synthesizing ent-kaurene from ent-copalyl diphosphate; a gene encoding a polypeptide capable of glycosylating steviol or a steviol glycoside at its C-13 hydroxyl position; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating steviol or a steviol glycoside at its C-19 carboxyl position; and/or a gene encoding a polypeptide capable of beta 1,2 glycosylation of the C2′ of the 13-O-glucose, 19-O-glucose, or both 13-O-glucose and 19-O-glucose of a steviol glycoside can produce a steviol glycoside and/or steviol glycoside precursors in vivo. See, e.g., FIGS. 1 and 2. The skilled worker will appreciate that one or more of these genes can be endogenous to the host provided that at least one (and in some embodiments, all) of these genes is a recombinant gene introduced into the recombinant host.

In some aspects, the polypeptide capable of synthesizing GGPP from FPP and IPP comprises a polypeptide having an amino acid sequence set forth in SEQ ID NO:20 (which can be encoded by the nucleotide sequence set forth in SEQ ID NO:19), SEQ ID NO:22 (encoded by the nucleotide sequence set forth in SEQ ID NO:21), SEQ ID NO:24 (encoded by the nucleotide sequence set forth in SEQ ID NO:23), SEQ ID NO:26 (encoded by the nucleotide sequence set forth in SEQ ID NO:25), SEQ ID NO:28 (encoded by the nucleotide sequence set forth in SEQ ID NO:27), SEQ ID NO:30 (encoded by the nucleotide sequence set forth in SEQ ID NO:29), SEQ ID NO:32 (encoded by the nucleotide sequence set forth in SEQ ID NO:31), or SEQ ID NO:116 (encoded by the nucleotide sequence set forth in SEQ ID NO:115).

In some aspects, the polypeptide capable of synthesizing ent-copalyl diphosphate from GGPP comprises a polypeptide having an amino acid sequence set forth in SEQ ID NO:34 (which can be encoded by the nucleotide sequence set forth in SEQ ID NO:33), SEQ ID NO:36 (encoded by the nucleotide sequence set forth in SEQ ID NO:35), SEQ ID NO:38 (encoded by the nucleotide sequence set forth in SEQ ID NO:37), SEQ ID NO:40 (encoded by the nucleotide sequence set forth in SEQ ID NO:39), or SEQ ID NO:42 (encoded by the nucleotide sequence set forth in SEQ ID NO:41). In some embodiments, the polypeptide capable of synthesizing ent-copalyldiphosphate from GGPP lacks a chloroplast transit peptide.

In some aspects, the polypeptide capable of synthesizing ent-kaurene from ent-copalyl pyrophosphate comprises a polypeptide having an amino acid sequence set forth in SEQ ID NO:44 (which can be encoded by the nucleotide sequence set forth in SEQ ID NO:43), SEQ ID NO:46 (encoded by the nucleotide sequence set forth in SEQ ID NO:45), SEQ ID NO:48 (encoded by the nucleotide sequence set forth in SEQ ID NO:47), SEQ ID NO:50 (encoded by the nucleotide sequence set forth in SEQ ID NO:49), or SEQ ID NO:52 (encoded by the nucleotide sequence set forth in SEQ ID NO:51).

In some embodiments, a recombinant host comprises a gene encoding a bifunctional polypeptide capable of synthesizing ent-copalyl diphosphate from GGPP and synthesizing ent-kaurene from ent-copalyl pyrophosphate. In some aspects, the bifunctional polypeptide comprises a polypeptide having an amino acid sequence set forth in SEQ ID NO:54 (which can be encoded by the nucleotide sequence set forth in SEQ ID NO:53), SEQ ID NO:56 (encoded by the nucleotide sequence set forth in SEQ ID NO:55), or SEQ ID NO:58 (encoded by the nucleotide sequence set forth in SEQ ID NO:57).

In some aspects, the polypeptide capable of synthesizing ent-kaurenoic acid, ent-kaurenol, and/or ent-kaurenol from ent-kaurene comprises a polypeptide having an amino acid sequence set forth in SEQ ID NO:60 (which can be encoded by the nucleotide sequence set forth in SEQ ID NO:59), SEQ ID NO:62 (encoded by the nucleotide sequence set forth in SEQ ID NO:61), SEQ ID NO:117 (encoded by the nucleotide sequence set forth in SEQ ID NO:63 or SEQ ID NO:64), SEQ ID NO:66 (encoded by the nucleotide sequence set forth in SEQ ID NO:65), SEQ ID NO:68 (encoded by the nucleotide sequence set forth in SEQ ID NO:67), SEQ ID NO:70 (encoded by the nucleotide sequence set forth in SEQ ID NO:69), SEQ ID NO:72 (encoded by the nucleotide sequence set forth in SEQ ID NO:71), SEQ ID NO:74 (encoded by the nucleotide sequence set forth in SEQ ID NO:73), or SEQ ID NO:76 (encoded by the nucleotide sequence set forth in SEQ ID NO:75).

In some aspects, the polypeptide capable of reducing cytochrome P450 complex comprises a polypeptide having an amino acid sequence set forth in SEQ ID NO:78 (which can be encoded by the nucleotide sequence set forth in SEQ ID NO:77), SEQ ID NO:80 (encoded by the nucleotide sequence set forth in SEQ ID NO:79), SEQ ID NO:82 (encoded by the nucleotide sequence set forth in SEQ ID NO:81), SEQ ID NO:84 (encoded by the nucleotide sequence set forth in SEQ ID NO:83), SEQ ID NO:86 (encoded by the nucleotide sequence set forth in SEQ ID NO:85), SEQ ID NO:88 (encoded by the nucleotide sequence set forth in SEQ ID NO:87), SEQ ID NO:90 (encoded by the nucleotide sequence set forth in SEQ ID NO:89), or SEQ ID NO:92 (encoded by the nucleotide sequence set forth in SEQ ID NO:91).

In some aspects, the polypeptide capable of synthesizing steviol from ent-kaurenoic acid comprises a polypeptide having an amino acid sequence set forth in SEQ ID NO:94 (which can be encoded by the nucleotide sequence set forth in SEQ ID NO:93), SEQ ID NO:97 (encoded by the nucleotide sequence set forth in SEQ ID NO:95 or SEQ ID NO:96), SEQ ID NO:100 (encoded by the nucleotide sequence set forth in SEQ ID NO:98 or SEQ ID NO:99), SEQ ID NO:101, SEQ ID NO:102, SEQ ID NO:103, SEQ ID NO:104, SEQ ID NO:106 (encoded by the nucleotide sequence set forth in SEQ ID NO:105), SEQ ID NO:108 (encoded by the nucleotide sequence set forth in SEQ ID NO:107), SEQ ID NO:110 (encoded by the nucleotide sequence set forth in SEQ ID NO:109), SEQ ID NO:112 (encoded by the nucleotide sequence set forth in SEQ ID NO:111), or SEQ ID NO:114 (encoded by the nucleotide sequence set forth in SEQ ID NO:113).

In some embodiments, a recombinant host comprises a nucleic acid encoding a polypeptide capable of glycosylating steviol or a steviol glycoside at its C-13 hydroxyl position, a nucleic acid encoding a polypeptide capable of beta 1,3 glycosylation of the C3′ of the 13-O-glucose, 19-O-glucose, or both 13-O-glucose and 19-O-glucose of a steviol glycoside, a nucleic acid encoding a polypeptide capable of glycosylating steviol or a steviol glycoside at its C-19 carboxyl position, a nucleic acid encoding a polypeptide capable of beta 1,2 glycosylation of the C2′ of the 13-O-glucose, 19-O-glucose, or both 13-O-glucose and 19-O-glucose of a steviol glycoside. In certain such embodiments, the recombinant host further comprises a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; and/or a gene encoding a bifunctional polypeptide capable of synthesizing ent-copalyl diphosphate from GGPP and synthesizing ent-kaurene from ent-copalyl diphosphate.

In some embodiments, a recombinant host comprises a gene encoding a polypeptide capable of glycosylating steviol or a steviol glycoside at its C-19 carboxyl position, e.g., a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, and/or a UGT74F2-like UGT polypeptide. In certain such embodiments, the recombinant host further comprises a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a bifunctional polypeptide capable of synthesizing ent-copalyl diphosphate from GGPP and synthesizing ent-kaurene from ent-copalyl diphosphate; a gene encoding a polypeptide capable of glycosylating steviol or a steviol glycoside at its C-13 hydroxyl position; a gene encoding a polypeptide capable of beta 1,3 glycosylation of the C3′ of the 13-O-glucose, 19-O-glucose, or both 13-O-glucose and 19-O-glucose of a steviol glycoside; and/or a gene encoding a polypeptide capable of beta 1,2 glycosylation of the C2′ of the 13-O-glucose, 19-O-glucose, or both 13-O-glucose and 19-O-glucose of a steviol glycoside.

In some embodiments, a recombinant host comprises a gene encoding a polypeptide capable of glycosylating steviol or a steviol glycoside at its C-13 hydroxyl position, e.g., a UGT73C1 polypeptide, a UGT73C3 polypeptide, a UGT73C5 polypeptide, a UGT73C6 polypeptide, a UGT73C7 polypeptide, a UGT73E1 polypeptide, and/or a UGT76E12 polypeptide. In certain such embodiments, the recombinant host further comprises a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a bifunctional polypeptide capable of synthesizing ent-copalyl diphosphate from GGPP and synthesizing ent-kaurene from ent-copalyl diphosphate; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating steviol or a steviol glycoside at its C-19 carboxyl position; and/or a gene encoding a polypeptide capable of beta 1,2 glycosylation of the C2′ of the 13-O-glucose, 19-O-glucose, or both 13-O-glucose and 19-O-glucose of a steviol glycoside.

In some embodiments, a recombinant host comprises a gene encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside (that is, examples of glycosyl-position glycosylation), e.g., a UGT73C6 polypeptide, a CaUGT3 polypeptide, a UN32491 polypeptide, and/or a UN1671 polypeptide. In certain such embodiments, the recombinant host further comprises a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a bifunctional polypeptide capable of synthesizing ent-copalyl diphosphate from GGPP and synthesizing ent-kaurene from ent-copalyl diphosphate; a gene encoding a polypeptide capable of glycosylating steviol or a steviol glycoside at its C-13 hydroxyl position; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating steviol or a steviol glycoside at its C-19 carboxyl position; and/or a gene encoding a polypeptide capable of beta 1,2 glycosylation of the C2′ of the 13-O-glucose, 19-O-glucose, or both 13-O-glucose and 19-O-glucose of a steviol glycoside.

In some embodiments, a recombinant host comprises a gene encoding a polypeptide capable of glycosylating a steviol precursor at its C-19 carboxyl or C-19 hydroxyl position, e.g., a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a UGT76E12 polypeptide, a Olel polypeptide, a UGTS polypeptide, a SA Gtase, a UDPG1 polypeptide, a UGT74F1 polypeptide, a UGT75D1 polypeptide, a UGT84B2 polypeptide, and/or a UGT74F2-like UGT polypeptide. In certain such embodiments, the recombinant host further comprises a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a bifunctional polypeptide capable of synthesizing ent-copalyl diphosphate from GGPP and synthesizing ent-kaurene from ent-copalyl diphosphate; a gene encoding a polypeptide capable of glycosylating steviol or a steviol glycoside at its C-13 hydroxyl position; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating steviol or a steviol glycoside at its C-19 carboxyl position; and/or a gene encoding a polypeptide capable of beta 1,2 glycosylation of the C2′ of the 13-O-glucose, 19-O-glucose, or both 13-O-glucose and 19-O-glucose of a steviol glycoside.

In some embodiments, a recombinant host comprises a nucleic acid encoding a polypeptide capable of glycosylating steviol or a steviol glycoside at its C-13 hydroxyl position (e.g., UGT85C2 polypeptide) (SEQ ID NO:7), a nucleic acid encoding a polypeptide capable of beta 1,3 glycosylation of the C3′ of the 13-O-glucose, 19-O-glucose, or both 13-O-glucose and 19-O-glucose of a steviol glycoside (e.g., UGT76G1 polypeptide) (SEQ ID NO:9), a nucleic acid encoding a polypeptide capable of glycosylating steviol or a steviol glycoside at its C-19 carboxyl position (e.g., UGT74G1 polypeptide) (SEQ ID NO:4), a nucleic acid encoding a polypeptide capable of beta 1,2 glycosylation of the C2′ of the 13-O-glucose, 19-O-glucose, or both 13-O-glucose and 19-O-glucose of a steviol glycoside (e.g., EUGT11 polypeptide) (SEQ ID NO:16). In some aspects, the polypeptide capable of beta 1,2 glycosylation of the C2′ of the 13-O-glucose, 19-O-glucose, or both 13-O-glucose and 19-O-glucose of a steviol glycoside (e.g., UGT91D2 polypeptide) can be a UGT91D2e polypeptide (SEQ ID NO:11) or a UGT91D2e-b polypeptide (SEQ ID NO:13).

In some aspects, the polypeptide capable of glycosylating steviol or a steviol glycoside at its C-13 hydroxyl position is encoded by the nucleotide sequence set forth in SEQ ID NO:5 or SEQ ID NO:6, the polypeptide capable of beta 1,3 glycosylation of the C3′ of the 13-O-glucose, 19-O-glucose, or both 13-O-glucose and 19-O-glucose of a steviol glycoside is encoded by the nucleotide sequence set forth in SEQ ID NO:8, the polypeptide capable of glycosylating steviol or a steviol glycoside at its C-19 carboxyl position is encoded by the nucleotide sequence set forth in SEQ ID NO:3, the polypeptide capable of beta 1,2 glycosylation of the C2′ of the 13-O-glucose, 19-O-glucose, or both 13-O-glucose and 19-O-glucose of a steviol glycoside is encoded by the nucleotide sequence set forth in SEQ ID NO:10,12,14 or 15. The skilled worker will appreciate that expression of these genes may be necessary to produce a particular steviol glycoside but that one or more of these genes can be endogenous to the host provided that at least one (and in some embodiments, all) of these genes is a recombinant gene introduced into the recombinant host.

In a particular embodiment, a steviol-producing recombinant microorganism comprises exogenous nucleic acids encoding a polypeptide capable of glycosylating steviol or a steviol glycoside at its C-13 hydroxyl position, a polypeptide capable of beta 1,3 glycosylation of the C3′ of the 13-O-glucose, 19-O-glucose, or both 13-O-glucose and 19-O-glucose of a steviol glycoside, and a polypeptide capable of beta 1,2 glycosylation of the C2′ of the 13-O-glucose, 19-O-glucose, or both 13-O-glucose and 19-O-glucose of a steviol glycoside polypeptides.

In another particular embodiment, a steviol-producing recombinant microorganism comprises exogenous nucleic acids encoding a polypeptide capable of glycosylating steviol or a steviol glycoside at its C-13 hydroxyl position; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating steviol or a steviol glycoside at its C-19 carboxyl position; and a polypeptide capable of beta 1,2 glycosylation of the C2′ of the 13-O-glucose, 19-O-glucose, or both 13-O-glucose and 19-O-glucose of a steviol glycoside.

In some embodiments, polypeptides capable of catalyzing the 19-O-glycosylation of ent-kaurenoic acid (KA) to ent-kaurenoic acid+1Glc (#58), in vitro, in a recombinant host, or by whole cell bioconversion include UGT73C1 (SEQ ID NO:127), UGT73C3 (SEQ ID NO:133), UGT73C5 (SEQ ID NO:135), UGT73C6 (SEQ ID NO:137), UGT73E1 (SEQ ID NO:141), UGT74G1 (SEQ ID NO:4), UGT75B1 (SEQ ID NO:145), UGT75L6 (SEQ ID NO:147), UGT76E12 (SEQ ID NO:153), Olel (SEQ ID NO:177), UGTS (SEQ ID NO:181), SA Gtase (SEQ ID NO:183), UDPG1 (SEQ ID NO:185), UGT74F1 (SEQ ID NO:203), UGT75D1 (SEQ ID NO:205), UGT84B2 (SEQ ID NO:207), CaUGT2 (SEQ ID NO:209), and a UGT74F2-like UGT polypeptide (SEQ ID NO:211). See, Example 3.

In some embodiments, polypeptides capable of catalyzing the 13-O-glycosylation of steviol to 13-SMG, in vitro, in a recombinant host, or by whole cell bioconversion include UGT73C1 (SEQ ID NO:127), UGT73C3 (SEQ ID NO:133), UGT73C5 (SEQ ID NO:135), UGT73C6 (SEQ ID NO:137), UGT73C7 (SEQ ID NO:139), UGT73E1 (SEQ ID NO:141), UGT76E12 (SEQ ID NO:153), and UGT85C2 (SEQ ID NO:7). See, Example 3.

In some embodiments, polypeptides capable of catalyzing the 19-O-glycosylation of steviol to 19-SMG, in vitro, in a recombinant host, or by whole cell bioconversion include UGT73C1 (SEQ ID NO:127), UGT73C3 (SEQ ID NO:133), UGT73C5 (SEQ ID NO:135), UGT73C6 (SEQ ID NO:137), UGT73E1 (SEQ ID NO:141), UGT74D1 (SEQ ID NO:143), UGT74G1 (SEQ ID NO:4), UGT75B1 (SEQ ID NO:145), UGT75L6 (SEQ ID NO:147), Olel (SEQ ID NO:177), UGT5 (SEQ ID NO:181), SA Gtase (SEQ ID NO:183), and UDPG1 (SEQ ID NO:185). See, Example 3.

In some embodiments, polypeptides capable of catalyzing the 19-O-glycosylation of 13-SMG to rubusoside, in vitro, in a recombinant host, or by whole cell bioconversion include UGT73C1 (SEQ ID NO:127), UGT73C6 (SEQ ID NO:137), UGT74G1 (SEQ ID NO:4), UGT85C2 (SEQ ID NO:7), SA Gtase (SEQ ID NO:183), UDPG1 (SEQ ID NO:185), UN1671 (SEQ ID NO:201), UGT74F1 (SEQ ID NO:203), UGT75D1 (SEQ ID NO:205), UGT84B2 (SEQ ID NO:207), CaUGT2 (SEQ ID NO:209), and a UGT74F2-like UGT polypeptide (SEQ ID NO:211). See, Example 3.

In some embodiments, polypeptides capable of catalyzing the glycosylation of 13-SMG (that is, an examples of glycosyl-position glycosylation) to steviol-1,2-bioside, in vitro, in a recombinant host, or by whole cell bioconversion include UGT91D2e-b (SEQ ID NO:13), EUGT11 (SEQ ID NO:16), and UN32491 (SEQ ID NO:199).

In some embodiments, polypeptides capable of catalyzing the glycosyl-position glycosylation of rubusoside to 1,2-stevioside, in vitro, in a recombinant host, or by whole cell bioconversion include UGT73C6 (SEQ ID NO:137), UGT91D2e-b (SEQ ID NO:13), CaUGT3 (SEQ ID NO:169), and EUGT11 (SEQ ID NO:16). See, Example 3.

In some embodiments, polypeptides capable of catalyzing the glycosyl-position glycosylation of rubusoside to steviol+3Glc (#55), in vitro, in a recombinant host, or by whole cell bioconversion include EUGT11 (SEQ ID NO:16).

In some embodiments, polypeptides capable of catalyzing the 19-O-glycosylation of RebB to RebA, in vitro, in a recombinant host, or by whole cell bioconversion include UGT74G1 (SEQ ID NO:4). See, Example 3.

In some embodiments, polypeptides capable of catalyzing the glycosyl-position glycosylation of RebA to RebD, in vitro, in a recombinant host, or by whole cell bioconversion include EUGT11 (SEQ ID NO:16).

In some embodiments, polypeptides capable of catalyzing the glycosyl-position glycosylation of RebA to steviol+5Glc (#24), in vitro, in a recombinant host, or by whole cell bioconversion include EUGT11 (SEQ ID NO:16) and UN1671 (SEQ ID NO:201). See, Example 3.

In some aspects, polypeptides capable of 19-O-glycosylation activity on steviol, steviol glycosides, and precurors thereof in vitro, in a recombinant host, or by whole cell bioconversion include UGT73C1 (SEQ ID NO:127), UGT73C3 (SEQ ID NO:133), UGT73C5 (SEQ ID NO:135), UGT73C6 (SEQ ID NO:137), UGT73E1 (SEQ ID NO:141), UGT74G1 (SEQ ID NO:4), UGT85C2 (SEQ ID NO:7), UGT75B1 (SEQ ID NO:145), UGT75L6 (SEQ ID NO:147), UGT76E12 (SEQ ID NO:153), Olel (SEQ ID NO:177), UGT5 (SEQ ID NO:181), SA Gtase (SEQ ID NO:183), UDPG1 (SEQ ID NO:185), UN1671 (SEQ ID NO:201), UGT74F1 (SEQ ID NO:203), UGT75D1 (SEQ ID NO:205), UGT84B2 (SEQ ID NO:207), and a UGT74F2-like UGT (SEQ ID NO:211). See, Example 3. Non-limiting examples of 19-O-glycosylation reactions include conversion of ent-kaurenoic acid to ent-kaurenoic acid+1Glc (#58), conversion of 13-SMG to rubusoside, and/or conversion of steviol to 19-SMG (see, e.g., FIG. 1).

In some aspects, polypeptides capable of 13-O-glycosylation activity on steviol and steviol glycosides in vitro, in a recombinant host, or by whole cell bioconversion include UGT73C1 (SEQ ID NO:127), UGT73C3 (SEQ ID NO:133), UGT73C5 (SEQ ID NO:135), UGT73C6 (SEQ ID NO:137), UGT73C7 (SEQ ID NO:139), UGT73E1 (SEQ ID NO:141), UGT76E12 (SEQ ID NO:153), and UGT85C2 (SEQ ID NO:7). See, Example 3. A non-limiting example of a 13-O-glycosylation reaction includes conversion of steviol to 13-SMG (see, e.g., FIG. 1).

In some aspects, polypeptides capable of glycosylation activity towards the glucose residues of steviol glycosides including, but not limited to, catalyzing the conversion of 13-SMG to steviol-1,2-bioside, catalyzing the conversion of rubusoside to 1,2-stevioside, and/or catalyzing the conversion of RebA to steviol+5Glc (#24) (see, e.g., FIG. 1), in vitro, in a recombinant host, or by whole cell bioconversion include UGT73C6 (SEQ ID NO:137), UGT91D2e-b (SEQ ID NO:13), CaUGT3 (SEQ ID NO:169), EUGT11 (SEQ ID NO:16), UN32491 (SEQ ID NO:199), and UN1671 (SEQ ID NO:201). See, Example 3.

In some embodiments, a recombinant host comprises a nucleic acid encoding a UGT85C2 polypeptide (SEQ ID NO:7), a nucleic acid encoding a UGT76G1 polypeptide (SEQ ID NO:9), a nucleic acid encoding a UGT74G1 polypeptide (SEQ ID NO:4), a nucleic acid encoding a UGT91D2 polypeptide, and/or a nucleic acid encoding a EUGT11 polypeptide (SEQ ID NO:16). In some aspects, the UGT91D2 polypeptide can be a UGT91D2e polypeptide (SEQ ID NO:11) a UGT91D2e-b polypeptide (SEQ ID NO:13). In some embodiments, a recombinant host comprises a nucleic acid encoding a UGT73C1 polypeptide (SEQ ID NO:127), a nucleic acid encoding a UGT73C3 polypeptide (SEQ ID NO:133), a nucleic acid encoding a UGT73C5 polypeptide (SEQ ID NO:135), a nucleic acid encoding a UGT73C6 polypeptide (SEQ ID NO:137), a nucleic acid encoding a UGT73C7 polypeptide (SEQ ID NO:139), a nucleic acid encoding a UGT73E1 polypeptide (SEQ ID NO:141), a nucleic acid encoding a UGT74D1 polypeptide (SEQ ID NO:143), a nucleic acid encoding a UGT75B1 polypeptide (SEQ ID NO:145), a nucleic acid encoding a UGT75L6 polypeptide (SEQ ID NO:147), a nucleic acid encoding a UGT76E12 polypeptide (SEQ ID NO:153), a nucleic acid encoding a CaUGT3 polypeptide (SEQ ID NO:169), a nucleic acid encoding a Olel polypeptide (SEQ ID NO:177), a nucleic acid encoding a UGT5 (SEQ ID NO:181), a nucleic acid encoding a SA Gtase polypeptide (SEQ ID NO:183), a nucleic acid encoding a UDPG1 polypeptide (SEQ ID NO:185), a nucleic acid encoding a UN32491 polypeptide (SEQ ID NO:199), a nucleic acid encoding a UN1671 polypeptide (SEQ ID NO:201), a nucleic acid encoding a UGT74F1 polypeptide (SEQ ID NO:203), a nucleic acid encoding a UGT75D1 polypeptide (SEQ ID NO:205), a nucleic acid encoding a UGT84B2 polypeptide (SEQ ID NO:207), a nucleic acid encoding a CaUGT2 polypeptide (SEQ ID NO:209) or a nucleic acid encoding a UGT74F2-like UGT polypeptide (SEQ ID NO:211).

In some aspects, the UGT85C2 polypeptide is encoded by the nucleotide sequence set forth in SEQ ID NO:5, SEQ ID NO:6 the UGT76G1 polypeptide is encoded by the nucleotide sequence set forth in SEQ ID NO:8, the UGT74G1 polypeptide is encoded by the nucleotide sequence set forth in SEQ ID NO:3 or SEQ ID NO:213, the UGT91D2e polypeptide is encoded by the nucleotide sequence set forth in SEQ ID NO:10, the UGT91D2e-b polypeptide is encoded by the nucleotide sequence set forth in SEQ ID NO:12 or SEQ ID NO:212, the EUGT11 polypeptide is encoded by the nucleotide sequence set forth in SEQ ID NO:14 or SEQ ID NO:15, the UGT73C1 polypeptide is encoded by the nucleotide sequence set forth in SEQ ID NO:126, the UGT73C3 polypeptide is encoded by the nucleotide sequence set forth in SEQ ID NO:132, the UGT73C5 polypeptide is encoded by the nucleotide sequence set forth in SEQ ID NO:134, the UGT73C6 polypeptide is encoded by the nucleotide sequence set forth in SEQ ID NO:136, the UGT73C7 polypeptide is encoded by the nucleotide sequence set forth in SEQ ID NO:138, the UGT73E1 polypeptide is encoded by the nucleotide sequence set forth in SEQ ID NO:140, the UGT74D1 polypeptide is encoded by the nucleotide sequence set forth in SEQ ID NO:142, the UGT75B1 polypeptide is encoded by the nucleotide sequence set forth in SEQ ID NO:144, the UGT75L6 polypeptide is encoded by the nucleotide sequence set forth in SEQ ID NO:146, the UGT76E12 polypeptide is encoded by the nucleotide sequence set forth in SEQ ID NO:152, the CaUGT3 polypeptide is encoded by the nucleotide sequence set forth in SEQ ID NO:168, the Olel polypeptide is encoded by the nucleotide sequence set forth in SEQ ID NO:176, the UGT5 polypeptide is encoded by the nucleotide sequence set forth in SEQ ID NO:180, the SA Gtase polypeptide is encoded by the nucleotide sequence set forth in SEQ ID NO:182, the UDPG1 polypeptide is encoded by the nucleotide sequence set forth in SEQ ID NO:184, the UN32491 polypeptide is encoded by the nucleotide sequence set forth in SEQ ID NO:198, the UN1671 polypeptide is encoded by the nucleotide sequence set forth in SEQ ID NO:200, the UGT74F1 polypeptide is encoded by the nucleotide sequence set forth in SEQ ID NO:202, the UGT75D1 polypeptide is encoded by the nucleotide sequence set forth in SEQ ID NO:204, the UGT84B2 polypeptide is encoded by the nucleotide sequence set forth in SEQ ID NO:206, the CaUGT2 polypeptide is encoded by the nucleotide sequence set forth in SEQ ID NO:208, and the UGT74F2-like UGT polypeptide is encoded by the nucleotide sequence set forth in SEQ ID NO:210.

In some embodiments, steviol glycosides, glycosides of steviol precursors, and/or steviol glycoside precursors are produced through contact of a steviol glycoside precursor with one or more enzymes involved in the steviol glycoside pathway in vitro. For example, contacting steviol with one or more of a gene encoding a polypeptide capable of beta 1,3 glycosylation of the C3′ of the 13-O-glucose, 19-O-glucose, or both 13-O-glucose and 19-O-glucose of a steviol glycoside, a polypeptide capable of beta 1,2 glycosylation of the C2′ of the 13-O-glucose, 19-O-glucose, or both 13-O-glucose and 19-O-glucose of a steviol glycoside, and a polypeptide capable of glycosylating steviol or a steviol glycoside at its C-13 hydroxyl position or a polypeptide capable of glycosylating steviol or a steviol glycoside at its C-19 carboxyl position can result in production of a steviol glycoside in vitro. In some embodiments, a steviol glycoside precursor is produced through contact of an upstream steviol glycoside precursor with one or more enzymes involved in the steviol glycoside pathway in vitro. For example, contacting ent-kaurenoic acid with a polypeptide capable of synthesizing steviol from ent-kaurenoic acid can result in production of steviol in vitro.

In some embodiments, one or more steviol glycosides and/or glycosylated steviol precursors, or a composition thereof are produced in vitro. In some embodiments the method comprises adding a UGT85C2 polypeptide having at least 55% identity to an amino acid sequence set forth in SEQ ID NO:7; a UGT76G1 polypeptide having at least 50% identity to an amino acid sequence set forth in SEQ ID NO:9; a UGT74G1 polypeptide having at least 55% identity to an amino acid sequence set forth in SEQ ID NO:4; a UGT91D2 functional homolog polypeptide comprising a UGT91D2e polypeptide having 90% or greater identity to an amino acid sequence set forth in SEQ ID NO:11 or a UGT91D2e-b polypeptide having 90% or greater identity to an amino acid sequence set forth in SEQ ID NO:13; a EUGT11 polypeptide having at least 65% identity to an amino acid sequence set forth in SEQ ID NO:16; a UGT73C1 polypeptide comprises a polypeptide having at least 60% identity to an amino acid sequence set forth in SEQ ID NO:127; a UGT73C3 polypeptide comprises a polypeptide having at least 60% identity to an amino acid sequence set forth in SEQ ID NO:133; a UGT73C5 polypeptide comprises a polypeptide having at least 60% identity to an amino acid sequence set forth in SEQ ID NO:135; a UGT73C6 polypeptide comprises a polypeptide having at least 60% identity to an amino acid sequence set forth in SEQ ID NO:137; a UGT73E1 polypeptide comprises a polypeptide having at least 50% identity to an amino acid sequence set forth in SEQ ID NO:141; a UGT75B1 polypeptide comprises a polypeptide having at least 50% sequence identity to an amino acid sequence set forth in SEQ ID NO:145; a UGT75L6 polypeptide comprises a polypeptide having at least 60% sequence identity to an amino acid sequence set forth in SEQ ID NO:147; a UGT76E12 polypeptide comprises a polypeptide having at least 60% sequence identity to an amino acid sequence set forth in SEQ ID NO:153; a Olel polypeptide comprises a polypeptide having at least 55% identity to an amino acid sequence set forth in SEQ ID NO:177; a UGTS polypeptide comprises a polypeptide having at least 65% identity to an amino acid sequence set forth in SEQ ID NO:181; a SA Gtase polypeptide comprises a polypeptide having at least 55% identity to an amino acid sequence set forth in SEQ ID NO:183; a UDPG1 polypeptide comprises a polypeptide having at least 50% sequence identity to an amino acid sequence set forth in SEQ ID NO:185; a UN1671 polypeptide comprises a polypeptide having at least 45% identity to an amino acid sequence set forth in SEQ ID NO:201; a UGT74F1 polypeptide comprises a polypeptide having at least 50% sequence identity to an amino acid sequence set forth in SEQ ID NO:203; a UGT75D1 polypeptide comprises a polypeptide having at least 50% sequence identity to an amino acid sequence set forth in SEQ ID NO:205; a UGT84B2 polypeptide comprises a polypeptide having at least 40% sequence identity to an amino acid sequence set forth in SEQ ID NO:207; a UGT74F2-like UGT polypeptide comprises a polypeptide having at least 55% identity to an amino acid sequence set forth in SEQ ID NO:211; a UGT73C7 polypeptide comprises a polypeptide having at least 60% identity to an amino acid sequence set forth in SEQ ID NO:139; a CaUGT3 polypeptide comprises a polypeptide having at least 50% identity to an amino acid sequence set forth in SEQ ID NO:169; and/or a UN32491 polypeptide comprises a polypeptide having at least 50% identity to an amino acid sequence set forth in SEQ ID NO:199; and a plant-derived or synthetic steviol glycoside precursor or a plant-derived or synthetic steviol to a reaction mixture; wherein at least one of the polypeptides is a recombinant polypeptide; and producing the one or more steviol glycosides and/or glycosylated steviol precursors, or the composition thereof, thereby.

In some embodiments, a steviol glycoside or steviol glycoside precursor is produced by whole cell bioconversion. For whole cell bioconversion to occur, a host cell expressing one or more enzymes involved in the steviol glycoside pathway takes up and modifies the steviol glycoside or steviol glycoside precursor in the cell; following modification in vivo, the steviol glycoside or steviol glycoside precursor remains in the cell and/or is excreted into the cell culture medium. For example, a host cell expressing a gene encoding a polypeptide capable of glycosylating steviol or a steviol glycoside at its C-13 hydroxyl position; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating steviol or a steviol glycoside at its C-19 carboxyl position; and/or a gene encoding a polypeptide capable of beta 1,2 glycosylation of the C2′ of the 13-O-glucose, 19-O-glucose, or both 13-O-glucose and 19-O-glucose of a steviol glycoside can take up steviol and glycosylate steviol in the cell; following glycosylation in vivo, a steviol glycoside can be excreted into the culture medium. In certain such embodiments, the host cell may further express a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a polypeptide capable of synthesizing steviol from ent-kaurenoic acid; and/or a gene encoding a bifunctional polypeptide capable of synthesizing ent-copalyl diphosphate from GGPP and synthesizing ent-kaurene from ent-copalyl diphosphate.

In some embodiments, the method for producing one or more steviol glycosides and/or glycosylated steviol precursors, or a composition thereof as disclosed herein comprises whole cell bioconversion of a plant-derived or synthetic steviol glycoside precursor or a plant-derived or synthetic steviol precursor in a cell culture medium of a recombinant host cell using (a) a polypeptide capable of glycosylating steviol or a steviol glycoside at its C-19 carboxyl position; (b) a polypeptide capable of glycosylating steviol or a steviol glycoside at its C-13 hydroxyl position; (c) a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside (that is, examples of glycosyl-position glycosylation) activity on a steviol glycoside; and/or (d) a polypeptide is capable of glycosylating a steviol precursor at its C-19 carboxyl or C-19 hydroxyl position; wherein at least one of the polypeptide is a recombinant polypeptide expressed in the recombinant host cell, and producing the one or more steviol glycosides and/or glycosylated steviol precursors, or a composition thereof, thereby.

In some embodiments of the method for producing one or more steviol glycosides and/or glycosylated steviol precursors, or a composition thereof as disclosed herein by whole cell bioconversion of a plant-derived or synthetic steviol glycoside precursor or a plant-derived or synthetic steviol precursor in a cell culture medium of a recombinant host cell described herein, the polypeptide capable of glycosylating steviol or a steviol glycoside at its C-19 carboxyl position comprises a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, and/or a UGT74F2-like UGT polypeptide; the polypeptide capable of glycosylating steviol or a steviol glycoside at its C-13 hydroxyl position comprises a UGT73C1 polypeptide, a UGT73C3 polypeptide, a UGT73C5 polypeptide, a UGT73C6 polypeptide, a UGT73C7 polypeptide, a UGT73E1 polypeptide, and/or a UGT76E12 polypeptide; the polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside (that is, examples of glycosyl-position glycosylation) activity on a steviol glycoside comprises a UGT73C6 polypeptide, a CaUGT3 polypeptide, a UN32491 polypeptide, and/or a UN1671 polypeptide; and/or the polypeptide is capable of glycosylating a steviol precursor at its C-19 carboxyl or C-19 hydroxyl position comprises a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a UGT76E12 polypeptide, a Olel polypeptide, a UGT5 polypeptide, a SA Gtase, a UDPG1 polypeptide, a UGT74F1 polypeptide, a UGT75D1 polypeptide, a UGT84B2 polypeptide, and/or a UGT74F2-like UGT polypeptide.

In some embodiments of the method for producing one or more steviol glycosides and/or glycosylated steviol precursors, or a composition thereof as disclosed herein by whole cell bioconversion of a plant-derived or synthetic steviol glycoside precursor or a plant-derived or synthetic steviol precursor in a cell culture medium of a recombinant host cell described herein, the UGT73C1 polypeptide comprises a polypeptide having at least 60% identity to an amino acid sequence set forth in SEQ ID NO:127, the UGT73C3 polypeptide comprises a polypeptide having at least 60% identity to an amino acid sequence set forth in SEQ ID NO:133, the UGT73C5 polypeptide comprises a polypeptide having at least 60% identity to an amino acid sequence set forth in SEQ ID NO:135, the UGT73C6 polypeptide comprises a polypeptide having at least 60% identity to an amino acid sequence set forth in SEQ ID NO:137, the UGT73E1 polypeptide comprises a polypeptide having at least 50% identity to an amino acid sequence set forth in SEQ ID NO:141, the UGT75B1 polypeptide comprises a polypeptide having at least 50% sequence identity to an amino acid sequence set forth in SEQ ID NO:145, the UGT75L6 polypeptide comprises a polypeptide having at least 60% sequence identity to an amino acid sequence set forth in SEQ ID NO:147, the UGT76E12 polypeptide comprises a polypeptide having at least 60% sequence identity to an amino acid sequence set forth in SEQ ID NO:153, the Olel polypeptide comprises a polypeptide having at least 55% identity to an amino acid sequence set forth in SEQ ID NO:177, the UGTS polypeptide comprises a polypeptide having at least 65% identity to an amino acid sequence set forth in SEQ ID NO:181, the SA Gtase polypeptide comprises a polypeptide having at least 55% identity to an amino acid sequence set forth in SEQ ID NO:183, the UDPG1 polypeptide comprises a polypeptide having at least 50% sequence identity to an amino acid sequence set forth in SEQ ID NO:185, the UN1671 polypeptide comprises a polypeptide having at least 45% identity to an amino acid sequence set forth in SEQ ID NO:201, the UGT74F1 polypeptide comprises a polypeptide having at least 50% sequence identity to an amino acid sequence set forth in SEQ ID NO:203, the UGT75D1 polypeptide comprises a polypeptide having at least 50% sequence identity to an amino acid sequence set forth in SEQ ID NO:205, the UGT84B2 polypeptide comprises a polypeptide having at least 40% sequence identity to an amino acid sequence set forth in SEQ ID NO:207, the UGT74F2-like UGT polypeptide comprises a polypeptide having at least 55% identity to an amino acid sequence set forth in SEQ ID NO:211, the UGT73C7 polypeptide comprises a polypeptide having at least 60% identity to an amino acid sequence set forth in SEQ ID NO:139, the CaUGT3 polypeptide comprises a polypeptide having at least 50% identity to an amino acid sequence set forth in SEQ ID NO:169, or the UN32491 polypeptide comprises a polypeptide having at least 50% identity to an amino acid sequence set forth in SEQ ID NO:199.

In some embodiments, a polypeptide, e.g., a UGT polypeptide, can be displayed on the surface of the recombinant host cells disclosed herein by fusing it with anchoring motifs.

In some embodiments, the cell is permeabilized to take up a substrate to be modified or to excrete a modified product. In some embodiments, a permeabilizing agent can be added to aid the feedstock entering into the host and product getting out. In some embodiments, the cells are permeabilized with a solvent such as toluene, or with a detergent such as Triton-X or Tween. In some embodiments, the cells are permeabilized with a surfactant, for example a cationic surfactant such as cetyltrimethylammonium bromide (CTAB). In some embodiments, the cells are permeabilized with periodic mechanical shock such as electroporation or a slight osmotic shock. For example, a crude lysate of the cultured microorganism can be centrifuged to obtain a supernatant. The resulting supernatant can then be applied to a chromatography column, e.g., a C18 column, and washed with water to remove hydrophilic compounds, followed by elution of the compound(s) of interest with a solvent such as methanol. The compound(s) can then be further purified by preparative HPLC. See also, WO 2009/140394.

In some embodiments, steviol, one or more steviol glycoside precursors, and/or one or more steviol glycosides are produced by co-culturing of two or more hosts. In some embodiments, one or more hosts, each expressing one or more enzymes involved in the steviol glycoside pathway, produce steviol, one or more steviol glycoside precursors, and/or one or more steviol glycosides. For example, a host expressing a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a polypeptide capable of synthesizing steviol from ent-kaurenoic acid; and/or a gene encoding a bifunctional polypeptide capable of synthesizing ent-copalyl diphosphate from GGPP and synthesizing ent-kaurene from ent-copalyl diphosphate and a host expressing a gene encoding a polypeptide capable of glycosylating steviol or a steviol glycoside at its C-13 hydroxyl position; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating steviol or a steviol glycoside at its C-19 carboxyl position; and/or a gene encoding a polypeptide capable of beta 1,2 glycosylation of the C2′ of the 13-O-glucose, 19-O-glucose, or both 13-O-glucose and 19-O-glucose of a steviol glycoside, produce one or more steviol glycosides.

In some embodiments, a recombinant host comprising a gene encoding a polypeptide capable of glycosylating steviol or a steviol glycoside at its C-19 carboxyl position, e.g., a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, and/or a UGT74F2-like UGT polypeptide further comprises a gene encoding a polypeptide capable of glycosylating steviol or a steviol glycoside at its C-13 hydroxyl position (e.g., a polypeptide having the amino acid sequence set forth in SEQ ID NO:7); a gene encoding a polypeptide capable of beta 1,3 glycosylation of the C3′ of the 13-O-glucose, 19-O-glucose, or both 13-O-glucose and 19-O-glucose of a steviol glycoside (e.g., a polypeptide having the amino acid sequence set forth in SEQ ID NO:9); a gene encoding a polypeptide capable of glycosylating steviol or a steviol glycoside at its C-19 carboxyl position (e.g., a polypeptide having the amino acid sequence set forth in SEQ ID NO:4); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a polypeptide having the amino acid sequence set forth in SEQ ID NO:11, SEQ ID NO:13, or SEQ ID NO:16). In certain such embodiments, the recombinant host cell further comprises a gene encoding a polypeptide capable of synthesizing GGPP from FPP and IPP (e.g., a polypeptide having the amino acid sequence set forth in SEQ ID NO:20); a gene encoding a polypeptide capable of synthesizing ent-copalyl diphosphate from GGPP (e.g., a polypeptide having the amino acid sequence set forth in SEQ ID NO:40); a gene encoding a polypeptide capable of synthesizing ent-kaurene from ent-copalyl diphosphate (e.g., a polypeptide having the amino acid sequence set forth in SEQ ID NO:52); a gene encoding a polypeptide capable of synthesizing ent-kaurenoic acid, ent-kaurenol, and/or ent-kaurenal from ent-kaurene (e.g., a polypeptide having the amino acid sequence set forth in SEQ ID NO:60 or SEQ ID NO:117); a gene encoding a polypeptide capable of reducing cytochrome P450 complex (e.g., a polypeptide having the amino acid sequence set forth in SEQ ID NO:78, SEQ ID NO:86, or SEQ ID NO:92); and/or a gene encoding a polypeptide capable of synthesizing steviol from ent-kaurenoic acid (e.g., a polypeptide having the amino acid sequence set forth in SEQ ID NO:94).

In some embodiments, a recombinant host comprising a gene encoding a polypeptide capable of glycosylating steviol or a steviol glycoside at its C-13 hydroxyl position, e.g., a UGT73C1 polypeptide, a UGT73C3 polypeptide, a UGT73C5 polypeptide, a UGT73C6 polypeptide, a UGT73C7 polypeptide, a UGT73E1 polypeptide, and/or a UGT76E12 polypeptide further comprises a gene encoding a polypeptide capable of glycosylating steviol or a steviol glycoside at its C-13 hydroxyl position (e.g., a polypeptide having the amino acid sequence set forth in SEQ ID NO:7); a gene encoding a polypeptide capable of beta 1,3 glycosylation of the C3′ of the 13-O-glucose, 19-O-glucose, or both 13-O-glucose and 19-O-glucose of a steviol glycoside (e.g., a polypeptide having the amino acid sequence set forth in SEQ ID NO:9); a gene encoding a polypeptide capable of glycosylating steviol or a steviol glycoside at its C-19 carboxyl position (e.g., a polypeptide having the amino acid sequence set forth in SEQ ID NO:4); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a polypeptide having the amino acid sequence set forth in SEQ ID NO:11, SEQ ID NO:13, or SEQ ID NO:16). In certain such embodiments, the recombinant host cell further comprises a gene encoding a polypeptide capable of synthesizing GGPP from FPP and IPP (e.g., a polypeptide having the amino acid sequence set forth in SEQ ID NO:20); a gene encoding a polypeptide capable of synthesizing ent-copalyl diphosphate from GGPP (e.g., a polypeptide having the amino acid sequence set forth in SEQ ID NO:40); a gene encoding a polypeptide capable of synthesizing ent-kaurene from ent-copalyl diphosphate (e.g., a polypeptide having the amino acid sequence set forth in SEQ ID NO:52); a gene encoding a polypeptide capable of synthesizing ent-kaurenoic acid, ent-kaurenol, and/or ent-kaurenal from ent-kaurene (e.g., a polypeptide having the amino acid sequence set forth in SEQ ID NO:60 or SEQ ID NO:117); a gene encoding a polypeptide capable of reducing cytochrome P450 complex (e.g., a polypeptide having the amino acid sequence set forth in SEQ ID NO:78, SEQ ID NO:86, or SEQ ID NO:92); and/or a gene encoding a polypeptide capable of synthesizing steviol from ent-kaurenoic acid (e.g., a polypeptide having the amino acid sequence set forth in SEQ ID NO:94).

In some embodiments, a recombinant host comprising a gene encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside (that is, examples of glycosyl-position glycosylation), e.g., a UGT73C6 polypeptide, a CaUGT3 polypeptide, a UN32491 polypeptide, and/or a UN1671 polypeptide further comprises a gene encoding a polypeptide capable of glycosylating steviol or a steviol glycoside at its C-13 hydroxyl position (e.g., a polypeptide having the amino acid sequence set forth in SEQ ID NO:7); a gene encoding a polypeptide capable of beta 1,3 glycosylation of the C3′ of the 13-O-glucose, 19-O-glucose, or both 13-O-glucose and 19-O-glucose of a steviol glycoside (e.g., a polypeptide having the amino acid sequence set forth in SEQ ID NO:9); a gene encoding a polypeptide capable of glycosylating steviol or a steviol glycoside at its C-19 carboxyl position (e.g., a polypeptide having the amino acid sequence set forth in SEQ ID NO:4); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a polypeptide having the amino acid sequence set forth in SEQ ID NO:11, SEQ ID NO:13, or SEQ ID NO:16). In certain such embodiments, the recombinant host cell further comprises a gene encoding a polypeptide capable of synthesizing GGPP from FPP and IPP (e.g., a polypeptide having the amino acid sequence set forth in SEQ ID NO:20); a gene encoding a polypeptide capable of synthesizing ent-copalyl diphosphate from GGPP (e.g., a polypeptide having the amino acid sequence set forth in SEQ ID NO:40); a gene encoding a polypeptide capable of synthesizing ent-kaurene from ent-copalyl diphosphate (e.g., a polypeptide having the amino acid sequence set forth in SEQ ID NO:52); a gene encoding a polypeptide capable of synthesizing ent-kaurenoic acid, ent-kaurenol, and/or ent-kaurenal from ent-kaurene (e.g., a polypeptide having the amino acid sequence set forth in SEQ ID NO:60 or SEQ ID NO:117); a gene encoding a polypeptide capable of reducing cytochrome P450 complex (e.g., a polypeptide having the amino acid sequence set forth in SEQ ID NO:78, SEQ ID NO:86, or SEQ ID NO:92); and/or a gene encoding a polypeptide capable of synthesizing steviol from ent-kaurenoic acid (e.g., a polypeptide having the amino acid sequence set forth in SEQ ID NO:94).

In some embodiments, a recombinant host comprising a gene encoding a polypeptide capable of glycosylating a steviol precursor at its C-19 carboxyl or C-19 hydroxyl position, e.g., a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a UGT76E12 polypeptide, a Olel polypeptide, a UGTS polypeptide, a SA Gtase, a UDPG1 polypeptide, a UGT74F1 polypeptide, a UGT75D1 polypeptide, a UGT84B2 polypeptide, and/or a UGT74F2-like UGT polypeptide further comprises a gene encoding a polypeptide capable of glycosylating steviol or a steviol glycoside at its C-13 hydroxyl position (e.g., a polypeptide having the amino acid sequence set forth in SEQ ID NO:7); a gene encoding a polypeptide capable of beta 1,3 glycosylation of the C3′ of the 13-O-glucose, 19-O-glucose, or both 13-O-glucose and 19-O-glucose of a steviol glycoside (e.g., a polypeptide having the amino acid sequence set forth in SEQ ID NO:9); a gene encoding a polypeptide capable of glycosylating steviol or a steviol glycoside at its C-19 carboxyl position (e.g., a polypeptide having the amino acid sequence set forth in SEQ ID NO:4); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a polypeptide having the amino acid sequence set forth in SEQ ID NO:11, SEQ ID NO:13, or SEQ ID NO:16). In certain such embodiments, the recombinant host cell further comprises a gene encoding a polypeptide capable of synthesizing GGPP from FPP and IPP (e.g., a polypeptide having the amino acid sequence set forth in SEQ ID NO:20); a gene encoding a polypeptide capable of synthesizing ent-copalyl diphosphate from GGPP (e.g., a polypeptide having the amino acid sequence set forth in SEQ ID NO:40); a gene encoding a polypeptide capable of synthesizing ent-kaurene from ent-copalyl diphosphate (e.g., a polypeptide having the amino acid sequence set forth in SEQ ID NO:52); a gene encoding a polypeptide capable of synthesizing ent-kaurenoic acid, ent-kaurenol, and/or ent-kaurenol from ent-kaurene (e.g., a polypeptide having the amino acid sequence set forth in SEQ ID NO:60 or SEQ ID NO:117); a gene encoding a polypeptide capable of reducing cytochrome P450 complex (e.g., a polypeptide having the amino acid sequence set forth in SEQ ID NO:78, SEQ ID NO:86, or SEQ ID NO:92); and/or a gene encoding a polypeptide capable of synthesizing steviol from ent-kaurenoic acid (e.g., a polypeptide having the amino acid sequence set forth in SEQ ID NO:94).

In some embodiments, a recombinant host comprising a gene encoding a polypeptide capable of glycosylating steviol or a steviol glycoside at its C-19 carboxyl position, e.g., a SA Gtase (e.g., a polypeptide having the amino acid sequence set forth in SEQ ID NO:183) further comprises a gene encoding a polypeptide capable of glycosylating steviol or a steviol glycoside at its C-13 hydroxyl position (e.g., a polypeptide having the amino acid sequence set forth in SEQ ID NO:7); a gene encoding a polypeptide capable of beta 1,3 glycosylation of the C3′ of the 13-O-glucose, 19-O-glucose, or both 13-O-glucose and 19-O-glucose of a steviol glycoside (e.g., a polypeptide having the amino acid sequence set forth in SEQ ID NO:9); a gene encoding a polypeptide capable of glycosylating steviol or a steviol glycoside at its C-19 carboxyl position (e.g., a polypeptide having the amino acid sequence set forth in SEQ ID NO:4); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a polypeptide having the amino acid sequence set forth in SEQ ID NO:11, SEQ ID NO:13, or SEQ ID NO:16). In certain such embodiments, the recombinant host cell further comprises a gene encoding a polypeptide capable of synthesizing GGPP from FPP and IPP (e.g., a polypeptide having the amino acid sequence set forth in SEQ ID NO:20); a gene encoding a polypeptide capable of synthesizing ent-copalyl diphosphate from GGPP (e.g., a polypeptide having the amino acid sequence set forth in SEQ ID NO:40); a gene encoding a polypeptide capable of synthesizing ent-kaurene from ent-copalyl diphosphate (e.g., a polypeptide having the amino acid sequence set forth in SEQ ID NO:52); a gene encoding a polypeptide capable of synthesizing ent-kaurenoic acid, ent-kaurenol, and/or ent-kaurenal from ent-kaurene (e.g., a polypeptide having the amino acid sequence set forth in SEQ ID NO:60 or SEQ ID NO:117); a gene encoding a polypeptide capable of reducing cytochrome P450 complex (e.g., a polypeptide having the amino acid sequence set forth in SEQ ID NO:78, SEQ ID NO:86, or SEQ ID NO:92); and/or a gene encoding a polypeptide capable of synthesizing steviol from ent-kaurenoic acid (e.g., a polypeptide having the amino acid sequence set forth in SEQ ID NO:94).

In some aspects, expression of SA Gtase (SEQ ID NO:182, SEQ ID NO:183) in S. cerevisiae comprising one or more copies of a recombinant gene encoding a GGPPS polypeptide (e.g., SEQ ID NO:19, SEQ ID NO:20), a recombinant gene encoding a truncated CDPS polypeptide (e.g., SEQ ID NO:39, SEQ ID NO:40), a recombinant gene encoding a KS polypeptide (e.g., SEQ ID NO:51, SEQ ID NO:52), a recombinant gene encoding a KO polypeptide (e.g., SEQ ID NO:59, SEQ ID NO:60), a recombinant gene encoding an ATR2 polypeptide (e.g., SEQ ID NO:91, SEQ ID NO:92), a recombinant gene encoding an EUGT11 polypeptide (e.g., SEQ ID NO:14/SEQ ID NO:15, SEQ ID NO:16), a recombinant gene encoding a KAH polypeptide (e.g., SEQ ID NO:93, SEQ ID NO:94), a recombinant gene encoding a CPR8 polypeptide (e.g., SEQ ID NO:85, SEQ ID NO:86), a recombinant gene encoding a UGT85C2 polypeptide (e.g., SEQ ID NO:5/SEQ ID NO:6/SEQ ID NO:149, SEQ ID NO:7) or a UGT85C2 variant (or functional homolog) of SEQ ID NO:7, a recombinant gene encoding a UGT74G1 polypeptide (e.g., SEQ ID NO:3, SEQ ID NO:4) of a UGT74G1 variant (or functional homolog) of SEQ ID NO:4, a recombinant gene encoding a UGT76G1 polypeptide (e.g., SEQ ID NO:8, SEQ ID NO:9) or a UGT76G1 variant (or functional homolog) of SEQ ID NO:9, and a recombinant gene encoding a UGT91D2e polypeptide (e.g., SEQ ID NO:10, SEQ ID NO:11) and/or a UGT91D2e variant (or functional homolog) of SEQ ID NO:11 such as a UGT91D2e-b (SEQ ID NO:12, SEQ ID NO:13) polypeptide results in increased ent-kaurenoic acid+2Glc (#7), ent-kaurenoic acid+3Glc (isomer 1), ent-kaurenoic acid+3Glc (isomer 2), 13-SMG, RebA, RebB, Steviol+4Glc (#36), Steviol+6Glc (isomer 1), Steviol+7Glc (isomer 2), and/or ent-Kaurenol+3Glc (isomer 1 and/or isomer 2). See, Example 4.

In some embodiments, a steviol glycoside and/or glycoside of a steviol precursor, or a composition thereof produced in vivo, in vitro, or by whole cell bioconversion comprises fewer contaminants or less of any particular contaminant than a stevia extract from, inter alia, a stevia plant. Contaminants can include plant-derived compounds that contribute to off-flavors. Potential contaminants include pigments, lipids, proteins, phenolics, saccharides, spathulenol and other sesquiterpenes, labdane diterpenes, monoterpenes, decanoic acid, 8,11,14-eicosatrienoic acid, 2-methyloctadecane, pentacosane, octacosane, tetracosane, octadecanol, stigmasterol, β-sitosterol, α-amyrin, β-amyrin, lupeol, β-amryin acetate, pentacyclic triterpenes, centauredin, quercitin, epi-alpha-cadinol, carophyllenes and derivatives, beta-pinene, beta-sitosterol, and gibberellin.

As used herein, the terms “detectable amount,” “detectable concentration,” “measurable amount,” and “measurable concentration” refer to a level of steviol glycosides measured in AUC, μM/OD₆₀₀, mg/L, μM, or mM. Steviol glycoside production (i.e., total, supernatant, and/or intracellular steviol glycoside levels) can be detected and/or analyzed by techniques generally available to one skilled in the art, for example, but not limited to, liquid chromatography-mass spectrometry (LC-MS), thin layer chromatography (TLC), high-performance liquid chromatography (HPLC), ultraviolet-visible spectroscopy/spectrophotometry (UV-Vis), mass spectrometry (MS), and NMR.

As used herein, the term “undetectable concentration” refers to a level of a compound that is too low to be measured and/or analyzed by techniques such as TLC, HPLC, UV-Vis, MS, or NMR. In some embodiments, a compound of an “undetectable concentration” is not present in a steviol glycoside or steviol glycoside precursor composition.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” In some embodiments, “and/or” is used to refer to the exogenous nucleic acids that a recombinant cell comprises, wherein a recombinant cell comprises one or more exogenous nucleic acids selected from a group. In some embodiments, “and/or” is used to refer to production of steviol glycosides and/or steviol glycoside precursors. In some embodiments, “and/or” is used to refer to production of steviol glycosides, wherein one or more steviol glycosides are produced. In some embodiments, “and/or” is used to refer to production of steviol glycosides, wherein one or more steviol glycosides are produced through one or more of the following steps: culturing a recombinant microorganism, synthesizing one or more steviol glycosides in a recombinant microorganism, and/or isolating one or more steviol glycosides.

### Functional Homologs

Functional homologs of the polypeptides described above are also suitable for use in producing steviol glycosides in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of steviol glycoside biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability as a steviol glycoside biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in steviol glycoside biosynthesis polypeptides, e.g., conserved functional domains. In some embodiments, nucleic acids and polypeptides are identified from transcriptome data based on expression levels rather than by using BLAST analysis.

Conserved regions can be identified by locating a region within the primary amino acid sequence of a steviol glycoside biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing steviol in a recombinant host include functional homologs of UGTs.

Methods to modify the substrate specificity of, for example, a UGT, are known to those skilled in the art, and include without limitation site-directed/rational mutagenesis approaches, random directed evolution approaches and combinations in which random mutagenesis/saturation techniques are performed near the active site of the enzyme. For example see Osmani et al., 2009, Phytochemistry 70: 325-347.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A % identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using the computer program Clustal Omega (version 1.2.1, default parameters), which allows alignments of nucleic acid or polypeptide sequences to be carried out across their entire length (global alignment). Chenna et al., 2003, Nucleic Acids Res. 31(13):3497-500.

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: % age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine a % identity of a candidate nucleic acid or amino acid sequence to a reference sequence, the sequences are aligned using Clustal Omega, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that functional UGT proteins can include additional amino acids that are not involved in the enzymatic activities carried out by the enzymes. In some embodiments, UGT proteins are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins. In some embodiments, a nucleic acid sequence encoding a UGT polypeptide can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, Conn.). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein. In some embodiments, a UGT polypeptide is altered by domain swapping.

In some embodiments, a fusion protein is a protein altered by circular permutation, which consists in the covalent attachment of the ends of a protein that would be opened elsewhere afterwards. Thus, the order of the sequence is altered without causing changes in the amino acids of the protein. In some embodiments, a targeted circular permutation can be produced, for example but not limited to, by designing a spacer to join the ends of the original protein. Once the spacer has been defined, there are several possibilities to generate permutations through generally accepted molecular biology techniques, for example but not limited to, by producing concatemers by means of PCR and subsequent amplification of specific permutations inside the concatemer or by amplifying discrete fragments of the protein to exchange to join them in a different order. The step of generating permutations can be followed by creating a circular gene by binding the fragment ends and cutting back at random, thus forming collections of permutations from a unique construct.

### Steviol and Steviol Glycoside Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism.

A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. “Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

One or more genes can be combined in a recombinant nucleic acid construct in “modules” useful for a discrete aspect of steviol and/or steviol glycoside production. Combining a plurality of genes in a module, particularly a polycistronic module, facilitates the use of the module in a variety of species. For example, a steviol biosynthesis gene cluster, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for steviol or steviol glycoside production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards steviol or steviol glycoside biosynthesis. For example, it may be desirable to downregulate synthesis of sterols in a yeast strain in order to further increase steviol or steviol glycoside production, e.g., by downregulating squalene epoxidase. As another example, it may be desirable to inhibit degradative functions of certain endogenous gene products, e.g., glycohydrolases that remove glucose moieties from secondary metabolites or phosphatases as discussed herein. In such cases, a nucleic acid that overexpresses the polypeptide or gene product may be included in a recombinant construct that is transformed into the strain. Alternatively, mutagenesis can be used to generate mutants in genes for which it is desired to increase or enhance function.

One aspect of the disclosure is an isolated nucleic acid molecule encoding a polypeptide capable of glycosylating steviol or a steviol glycoside at its C-19 carboxyl position or a catalytically active portion thereof. The nucleic acid is cDNA. In some embodiments, the encoded polypeptide capable of glycosylating steviol or a steviol glycoside at its C-19 carboxyl position or the catalytically active portion thereof comprises a a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, or a UGT74F2-like UGT polypeptide. In some embodiments, the encoded polypeptide capable of glycosylating steviol or a steviol glycoside at its C-19 carboxyl position or the catalytically active portion thereof comprises a polypeptide having the amino acid sequence set forth in SEQ ID NO:127, SEQ ID NO:133, SEQ ID NO:135, SEQ ID NO:137, SEQ ID NO:141, SEQ ID NO:145, SEQ ID NO:147, SEQ ID NO:177, SEQ ID NO:181, SEQ ID NO:183, SEQ ID NO:185, SEQ ID NO:201, SEQ ID NO:203, SEQ ID NO:207, or SEQ ID NO:211.

Another aspect of the disclosure is an isolated nucleic acid molecule encoding a polypeptide capable of glycosylating steviol or a steviol glycoside at its C-13 hydroxyl position or a catalytically active portion thereof. In some embodiments, the encoded polypeptide capable of glycosylating steviol or a steviol glycoside at its C-13 hydroxyl position or the catalytically active portion thereof comprises a UGT73C1 polypeptide, a UGT73C3 polypeptide, a UGT73C5 polypeptide, a UGT73C6 polypeptide, a UGT73C7 polypeptide, a UGT73E1 polypeptide, or a UGT76E12 polypeptide. In some embodiments, the encoded polypeptide capable of glycosylating steviol or a steviol glycoside at its C-13 hydroxyl position or the catalytically active portion thereof comprises a polypeptide having the amino acid sequence set forth in SEQ ID NO:127, SEQ ID NO:133, SEQ ID NO:135, SEQ ID NO:137, SEQ ID NO:139, SEQ ID NO:141, or SEQ ID NO:153.

Another aspect of the disclosure is an isolated nucleic acid molecule encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside or a catalytically active portion thereof. The nucleic acid is cDNA. In some embodiments, the encoded polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside or the catalytically active portion thereof comprises a UGT73C6 polypeptide, a CaUGT3 polypeptide, a UN32491 polypeptide, or a UN1671 polypeptide. In some embodiments, the encoded polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside or the catalytically active portion thereof comprises a polypeptide having the amino acid sequence set forth in SEQ ID NO: 137, SEQ ID NO:169, SEQ ID NO:199, or SEQ ID NO:201.

Another aspect of the disclosure is an isolated nucleic acid molecule encoding a polypeptide capable of glycosylating a steviol precursor at its C-19 carboxyl or C-19 hydroxyl position or a catalytically active portion thereof. The nucleic acid is cDNA. In some embodiments, the encoded polypeptide capable of glycosylating a steviol precursor at its C-19 carboxyl or C-19 hydroxyl position or the catalytically active portion thereof comprises a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a UGT76E12 polypeptide, a Olel polypeptide, a UGT5 polypeptide, a SA Gtase, a UDPG1 polypeptide, a UGT74F1 polypeptide, a UGT75D1 polypeptide, a UGT84B2 polypeptide, or a UGT74F2-like UGT polypeptide. In some embodiments, the encoded polypeptide capable of glycosylating a steviol precursor at its C-19 carboxyl or C-19 hydroxyl position or the catalytically active portion thereof comprises a polypeptide having the amino acid sequence set forth in SEQ ID NO: 127, SEQ ID NO:133, SEQ ID NO:135, SEQ ID NO:137, SEQ ID NO:141, SEQ ID NO:145, SEQ ID NO:147, SEQ ID NO:153, SEQ ID NO:177, SEQ ID NO:181, SEQ ID NO:183, SEQ ID NO:185, SEQ ID NO:203, SEQ ID NO:205, SEQ ID NO:207, or SEQ ID NO:211.

### Host Microorganisms

Recombinant hosts can be used to express polypeptides for the producing steviol glycosides, including mammalian, insect, plant, and algal cells. A number of prokaryotes and eukaryotes are also suitable for use in constructing the recombinant microorganisms described herein, e.g., gram-negative bacteria, yeast, and fungi. A species and strain selected for use as a steviol glycoside production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

Typically, the recombinant microorganism is grown in a fermenter at a temperature(s) for a period of time, wherein the temperature and period of time facilitate the production of a steviol glycoside. The constructed and genetically engineered microorganisms provided by the invention can be cultivated using conventional fermentation processes, including, inter alia, chemostat, batch, fed-batch cultivations, semi-continuous fermentations such as draw and fill, continuous perfusion fermentation, and continuous perfusion cell culture. Depending on the particular microorganism used in the method, other recombinant genes such as isopentenyl biosynthesis genes and terpene synthase and cyclase genes may also be present and expressed. Levels of substrates and intermediates, e.g., isopentenyl diphosphate, dimethylallyl diphosphate, GGPP, ent-kaurene and ent-kaurenoic acid, can be determined by extracting samples from culture media for analysis according to published methods.

Carbon sources of use in the instant method include any molecule that can be metabolized by the recombinant host cell to facilitate growth and/or production of the steviol glycosides. Examples of suitable carbon sources include, but are not limited to, sucrose (e.g., as found in molasses), fructose, xylose, ethanol, glycerol, glucose, cellulose, starch, cellobiose or other glucose-comprising polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

After the recombinant microorganism has been grown in culture for the period of time, wherein the temperature and period of time facilitate the production of a steviol glycoside, steviol and/or one or more steviol glycosides can then be recovered from the culture using various techniques known in the art. In some embodiments, a permeabilizing agent can be added to aid the feedstock entering into the host and product getting out. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC. See also, WO 2009/140394.

It will be appreciated that the various genes and modules discussed herein can be present in two or more recombinant hosts rather than a single host. When a plurality of recombinant hosts is used, they can be grown in a mixed culture to accumulate steviol and/or steviol glycosides.

Alternatively, the two or more hosts each can be grown in a separate culture medium and the product of the first culture medium, e.g., steviol, can be introduced into second culture medium to be converted into a subsequent intermediate, or into an end product such as, for example, RebA. The product produced by the second, or final host is then recovered. It will also be appreciated that in some embodiments, a recombinant host is grown using nutrient sources other than a culture medium and utilizing a system other than a fermenter.

Exemplary prokaryotic and eukaryotic species are described in more detail below. However, it will be appreciated that other species can be suitable. For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis, Rhodoturula mucilaginosa, Phaffia rhodozyma, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia bacteria cells, for example, Escherichia coli cells; Lactobacillus bacteria cells; Lactococcus bacteria cells; Comebacterium bacteria cells; Acetobacter bacteria cells; Acinetobacter bacteria cells; or Pseudomonas bacterial cells.

In some embodiments, a microorganism can be an Ascomycete such as Gibberella fujikuroi, Kluyveromyces lactis, Schizosaccharomyces pombe, Aspergillus niger, Yarrowia lipolytica, Ashbya gossypii, or S. cerevisiae.

In some embodiments, a microorganism can be an algal cell such as Blakeslea trispora, Dunaliella salina, Haematococcus pluvialis, Chlorella sp., Undaria pinnatifida, Sargassum, Laminaria japonica, Scenedesmus almeriensis species.

In some embodiments, a microorganism can be a cyanobacterial cell such as Blakeslea trispora, Dunaliella salina, Haematococcus pluvialis, Chlorefia sp., Undaria pinnatifida, Sargassum, Laminaria japonica, Scenedesmus almeriensis.

### Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

### Aspergillus spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for producing steviol glycosides.

E. coli

E. coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Agaricus, Gibberella, and Phanerochaete spp.

Agaricus, Gibberella, and Phanerochaete spp. can be useful because they are known to produce large amounts of isoprenoids in culture. Thus, the terpene precursors for producing large amounts of steviol glycosides are already produced by endogenous genes. Thus, modules comprising recombinant genes for steviol glycoside biosynthesis polypeptides can be introduced into species from such genera without the necessity of introducing mevalonate or MEP pathway genes.

Arxula adeninivorans (Blastobotrys adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications and is an oleaginous microorgamism. Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biochimie 91(6):692-6; Bankar et al., 2009, Appl Microbiol Biotechnol. 84(5):847-65.

### Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

Rhodosporidium toruloides

Rhodosporidium toruloides is oleaginous yeast and useful for engineering lipid-production pathways (See e.g. Zhu et al., 2013, Nature Commun. 3:1112; Ageitos et al., 2011, Applied Microbiology and Biotechnology 90(4):1219-27).

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

Kluyveromyces lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et al., 2014, N Biotechnol. 31(6):532-7.

### Physcomitrella spp.

Physcomitrella mosses, when grown in suspension culture, have characteristics similar to yeast or other fungal cultures. This genera can be used for producing plant secondary metabolites, which can be difficult to produce in other types of cells.

It will be appreciated that the recombinant host cell disclosed herein can comprise a plant cell, comprising a plant cell that is grown in a plant, a mammalian cell, an insect cell, a fungal cell, comprising a yeast cell, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species or is a Saccharomycete or is a Saccharomyces cerevisiae cell, an algal cell or a bacterial cell, comprising Escherichia cells, Lactobacillus cells, Lactococcus cells, Cornebacterium cells, Acetobacter cells, Acinetobacter cells, or Pseudomonas cells.

### Steviol Glycoside Compositions

Steviol glycosides do not necessarily have equivalent performance in different food systems. It is therefore desirable to have the ability to direct the synthesis to steviol glycoside compositions of choice. Recombinant hosts described herein can produce compositions that are selectively enriched for specific steviol glycosides (e.g., RebD or RebM) and have a consistent taste profile. As used herein, the term “enriched” is used to describe a steviol glycoside composition with an increased proportion of a particular steviol glycoside, compared to a steviol glycoside composition (extract) from a stevia plant. Thus, the recombinant hosts described herein can facilitate the production of compositions that are tailored to meet the sweetening profile desired for a given food product and that have a proportion of each steviol glycoside that is consistent from batch to batch. In some embodiments, hosts described herein do not produce or produce a reduced amount of undesired plant by-products found in Stevia extracts. Thus, steviol glycoside compositions produced by the recombinant hosts described herein are distinguishable from compositions derived from Stevia plants.

The amount of an individual steviol glycoside (e.g., RebA, RebB, RebD, or RebM) accumulated can be from about 1 to about 7,000 mg/L, e.g., about 1 to about 10 mg/L, about 3 to about 10 mg/L, about 5 to about 20 mg/L, about 10 to about 50 mg/L, about 10 to about 100 mg/L, about 25 to about 500 mg/L, about 100 to about 1,500 mg/L, or about 200 to about 1,000 mg/L, at least about 1,000 mg/L, at least about 1,200 mg/L, at least about at least 1,400 mg/L, at least about 1,600 mg/L, at least about 1,800 mg/L, at least about 2,800 mg/L, or at least about 7,000 mg/L. In some aspects, the amount of an individual steviol glycoside can exceed 7,000 mg/L. The amount of a combination of steviol glycosides (e.g., RebA, RebB, RebD, or RebM) accumulated can be from about 1 mg/L to about 7,000 mg/L, e.g., about 200 to about 1,500, at least about 2,000 mg/L, at least about 3,000 mg/L, at least about 4,000 mg/L, at least about 5,000 mg/L, at least about 6,000 mg/L, or at least about 7,000 mg/L. In some aspects, the amount of a combination of steviol glycosides can exceed 7,000 mg/L. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

It will be appreciated that the various genes and modules discussed herein can be present in two or more recombinant microorganisms rather than a single microorganism. When a plurality of recombinant microorganisms is used, they can be grown in a mixed culture to produce steviol and/or steviol glycosides. For example, a first microorganism can comprise one or more biosynthesis genes for producing a steviol glycoside precursor, while a second microorganism comprises steviol glycoside biosynthesis genes. The product produced by the second, or final microorganism is then recovered. It will also be appreciated that in some embodiments, a recombinant microorganism is grown using nutrient sources other than a culture medium and utilizing a system other than a fermenter.

Alternatively, the two or more microorganisms each can be grown in a separate culture medium and the product of the first culture medium, e.g., steviol, can be introduced into second culture medium to be converted into a subsequent intermediate, or into an end product such as RebA. The product produced by the second, or final microorganism is then recovered. It will also be appreciated that in some embodiments, a recombinant microorganism is grown using nutrient sources other than a culture medium and utilizing a system other than a fermenter.

Steviol glycosides and compositions obtained by the methods disclosed herein can be used to make food products, dietary supplements and sweetener compositions. See, e.g., WO 2011/153378, WO 2013/022989, WO 2014/122227, and WO 2014/122328.

For example, substantially pure steviol or steviol glycoside such as RebM or RebD can be included in food products such as ice cream, carbonated beverages, fruit juices, yogurts, baked goods, chewing gums, hard and soft candies, and sauces. Substantially pure steviol or steviol glycoside can also be included in non-food products such as pharmaceutical products, medicinal products, dietary supplements and nutritional supplements. Substantially pure steviol or steviol glycosides may also be included in animal feed products for both the agriculture industry and the companion animal industry. Alternatively, a mixture of steviol and/or steviol glycosides can be made by culturing recombinant microorganisms separately, each producing a specific steviol or steviol glycoside, recovering the steviol or steviol glycoside in substantially pure form from each microorganism and then combining the compounds to obtain a mixture comprising each compound in the desired proportion. The recombinant microorganisms described herein permit more precise and consistent mixtures to be obtained compared to current Stevia products.

In another alternative, a substantially pure steviol or steviol glycoside can be incorporated into a food product along with other sweeteners, e.g. saccharin, dextrose, sucrose, fructose, erythritol, aspartame, sucralose, monatin, or acesulfame potassium. The weight ratio of steviol or steviol glycoside relative to other sweeteners can be varied as desired to achieve a satisfactory taste in the final food product. See, eg., U.S. 2007/0128311. In some embodiments, the steviol or steviol glycoside may be provided with a flavor (e.g., citrus) as a flavor modulator.

Compositions produced by a recombinant microorganism described herein can be incorporated into food products. For example, a steviol glycoside composition produced by a recombinant microorganism can be incorporated into a food product in an amount ranging from about 20 mg steviol glycoside/kg food product to about 1800 mg steviol glycoside/kg food product on a dry weight basis, depending on the type of steviol glycoside and food product. For example, a steviol glycoside composition produced by a recombinant microorganism can be incorporated into a dessert, cold confectionary (e.g., ice cream), dairy product (e.g., yogurt), or beverage (e.g., a carbonated beverage) such that the food product has a maximum of 500 mg steviol glycoside/kg food on a dry weight basis. A steviol glycoside composition produced by a recombinant microorganism can be incorporated into a baked good (e.g., a biscuit) such that the food product has a maximum of 300 mg steviol glycoside/kg food on a dry weight basis. A steviol glycoside composition produced by a recombinant microorganism can be incorporated into a sauce (e.g., chocolate syrup) or vegetable product (e.g., pickles) such that the food product has a maximum of 1000 mg steviol glycoside/kg food on a dry weight basis. A steviol glycoside composition produced by a recombinant microorganism can be incorporated into bread such that the food product has a maximum of 160 mg steviol glycoside/kg food on a dry weight basis. A steviol glycoside composition produced by a recombinant microorganism, plant, or plant cell can be incorporated into a hard or soft candy such that the food product has a maximum of 1600 mg steviol glycoside/kg food on a dry weight basis. A steviol glycoside composition produced by a recombinant microorganism, plant, or plant cell can be incorporated into a processed fruit product (e.g., fruit juices, fruit filling, jams, and jellies) such that the food product has a maximum of 1000 mg steviol glycoside/kg food on a dry weight basis. In some embodiments, a steviol glycoside composition produced herein is a component of a pharmaceutical composition. See, e.g., Steviol Glycosides Chemical and Technical Assessment 69th JECFA, 2007, prepared by Harriet Wallin, Food Agric. Org.; EFSA Panel on Food Additives and Nutrient Sources added to Food (ANS), “Scientific Opinion on the safety of steviol glycosides for the proposed uses as a food additive,” 2010, EFSA Journal 8(4):1537; U.S. Food and Drug Administration GRAS Notice 323; U.S Food and Drug Administration GRAS Notice Notice 329; WO 2011/037959; WO 2010/146463; WO 2011/046423; and WO 2011/056834.

For example, such a steviol glycoside composition can have from 90-99 weight % RebA and an undetectable amount of stevia plant-derived contaminants, and be incorporated into a food product at from 25-1600 mg/kg, e.g., 100-500 mg/kg, 25-100 mg/kg, 250-1000 mg/kg, 50-500 mg/kg or 500-1000 mg/kg on a dry weight basis.

Such a steviol glycoside composition can be a RebB-enriched composition having greater than 3 weight % RebB and be incorporated into the food product such that the amount of RebB in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebB-enriched composition has an undetectable amount of stevia plant-derived contaminants.

Such a steviol glycoside composition can be a RebD-enriched composition having greater than 3 weight % RebD and be incorporated into the food product such that the amount of RebD in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebD-enriched composition has an undetectable amount of stevia plant-derived contaminants.

Such a steviol glycoside composition can be a RebE-enriched composition having greater than 3 weight % RebE and be incorporated into the food product such that the amount of RebE in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebE-enriched composition has an undetectable amount of stevia plant-derived contaminants.

Such a steviol glycoside composition can be a RebM-enriched composition having greater than 3 weight % RebM and be incorporated into the food product such that the amount of RebM in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebM-enriched composition has an undetectable amount of stevia plant-derived contaminants.

In some embodiments, a substantially pure steviol or steviol glycoside is incorporated into a tabletop sweetener or “cup-for-cup” product. Such products typically are diluted to the appropriate sweetness level with one or more bulking agents, e.g., maltodextrins, known to those skilled in the art. Steviol glycoside compositions enriched for RebA, RebB, RebD, RebE, or RebM, can be package in a sachet, for example, at from 10,000 to 30,000 mg steviol glycoside/kg product on a dry weight basis, for tabletop use. In some embodiments, a steviol glycoside produced in vitro, in vivo, or by whole cell bioconversion

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1

### LC-MS Analytical Procedures

LC-MS analyses were performed on Waters ACQUITY UPLC® (Waters Corporation) with a Waters ACQUITY UPLC® BEH C18 column (2.1×50 mm, 1.7 μm particles, 130 Å pore size) coupled to a Waters ACQUITY TQD triple quadropole mass spectrometer with electrospray ionization (ESI) in negative mode.

Compound separation for Method A was achieved by a gradient of the two mobile phases: A (water with 0.1% formic acid) and B (MeCN with 0.1% formic acid) by increasing from 20% to 50 % B between 0.3 to 2.0 min, increasing to 100% B at 2.01 min, holding 100% B for 0.6 min, and re-equilibrating for 0.6 min.

Compound separation for Method B was achieved by a gradient of the two mobile phases A (water with 0.1% formic acid) and B (MeCN with 0.1% formic acid) by increasing from 60% to 100% B in 2.5 min, holding 100% B for 0.1 min and re-equilibrating for 0.3 min.

The flow rate was 0.6 mL/min, and the column temperature was 55° C. Steviol glycosides were monitored using SIM (Single Ion Monitoring) and quantified by comparing with authentic standards. See Table 1 for m/z trace and retention time values of steviol glycosides detected.

TABLE 1
LC-MS Analytical Data for steviol and steviol glycosides.

| Compound | MS Trace | RT (min) | Method | FIG. | Table |
|---|---|---|---|---|---|
| steviol + 6Glc (isomer 1) | 1289.53 | 0.87 | A | 3 | |
| (also referred to as compound 6.1) | | | | | |
| steviol + 7Glc (isomer 2) | 1451.581 | 0.94 | A | 3 | |
| (also referred to as compound 7.2) | | | | | |
| RebD | 1127.48 | 1.08 | A | | |
| RebM | 1289.53 | 1.15 | A | | |
| steviol + 4Glc (#26) | 965.42 | 1.21 | A | 4 | |
| (also referred to as compound 4.26) | | | | | |
| steviol + 5Glc (#24) | 1127.48 | 1.18 | A | | 7 |
| (also referred to as compound 5.24) | | | | | |
| RebA | 965.42 | 1.43 | A | | |
| 1,2-stevioside | 803.37 | 1.43 | A | | 6 |
| rubusoside | 641.32 | 1.67 | A | | 5, 8 |
| RebB | 803.37 | 1.76 | A | | |
| steviol-1,2-bioside | 641.32 | 1.80 | A | | 5 |
| 19-SMG | 525.27 | 1.98 | A | | 4 |
| 13-SMG | 479.26 | 2.04 | A | | 4 |
| ent-kaurenoic acid + 3Glc (isomer 1) | 787.37 | 2.16 | A | 4 | |
| (also referred to as compound KA3.1) | | | | | |
| ent-kaurenoic acid + 3Glc (isomer 2) | 787.37 | 2.28 | A | 5 | |
| (also referred to as compound KA3.2) | | | | | |
| ent-kaurenol + 3Glc (isomer 1) | 773.4 | 2.36 | A | 5 | |
| co-eluted with ent-kaurenol + 3Glc (#6) | | | | | |
| (also referred to as compounds KL3.1 | | | | | |
| and KL3.6) | | | | | |
| ent-kaurenoic acid + 2Glc (#7) | 625.32 | 2.35 | A | | |
| (also referred to as compound KA2.7) | | | | | |
| steviol | 317.21 | 2.39 | A | | |
| ent-kaurenoic acid + 1Glc (#58) | 439.27 | 0.69 | B | | 3, 8 |
| [also referred to as compound | and | | | | |
| KA1.58] | 509.61 | | | | |

### Example 2

### Crude Lysate Preparation

Colonies of E. coli strains constructed to express a UGT polypeptide were placed into sterile 96 deep well plates with 1 mL of NZCYM bacterial culture broth comprising ampicillin. The plate was sealed and samples were allowed to grow overnight at 37° C., shaking at 200 rpm. The following day (i.e., Day 2), 50 μL of each culture was transferred to a new sterile 96 deep well plate with 1 mL of NZCYM bacterial culture broth comprising ampicillin and polypeptide expression inducers. The plate was sealed and samples were incubated at 20° C., shaking at 200 rpm for ˜20 h. On Day 3, the plate was centrifuged at 4000 rpm for 10 min at 4° C. After decanting the supernatant, 50 μL of a buffer comprising Tris-HCl, MgCl₂, CaCl₂, and protease inhibitors was added to each well and cells were resuspended by shaking at 200 rpm for 5 min at 4° C. The contents of each well (i.e., cell slurries) were then transferred to a PCR plate and sealed before freezing at −80° C. overnight. Frozen cell slurries were thawed at room temperature for up to 30 min. If the thawing mix was not viscous due to cell lysing, samples were frozen and thawed again. When samples were nearly thawed, 25 μL of binding buffer comprising DNase and MgCl₂ was added to each well. The PCR plate was incubated at room temperature for 5 min, shaking at 500 rpm, until samples became less viscous. Finally, samples were centrifuged at 4000 rpm for 5 min, after which the supernatants were used to measure UGT activity, as described in Example 3.

### Example 3

### UGT Activity Assay

UGT polypeptide samples prepared according to Example 2 were screened in vitro for activity on substrates including RebA, RebB, rubusoside, steviol, ent-kaurenoic acid, and 13-SMG by preparing a reaction mixture according to Table 2.

TABLE 2
UGT Activity Assay Reaction Mixture.

| Component | Volume (μL) |
|---|---|
| H₂O | 4.2 |
| Alkaline Phosphatase | 0.3 |
| 4× Buffer (10 mM Tris-HCl, 5 mM | 7.5 |
| MgCl₂, 1 mM CaCl₂) | |
| UDP-Glucose (1 mM) | 9 |
| Substrate | 3 |
| UGT Sample | 6 |

The reaction mixture was incubated overnight at 30° C. The reaction was stopped by adding 30 μL of 100% DMSO. The resultant mixture was diluted further with 90 μL 50% DMSO for LC-MS analysis according to Example 1. Both the products formed and the area-under-the-curve (AUC) values of each product are shown in Tables 3-7, organized by substrate.

TABLE 3
UGT Activity on ent-kaurenoic acid.

| | | Activity |
|---|---|---|
| | | ent-kaurenoic acid + 1Glc |
| UGT Polypeptide | SEQ ID NO: | (#58) Production (AUC) |
| UGT73C1 | 127 | 1095 |
| UGT73C3 | 133 | 227 |
| UGT73C5 | 135 | 2489 |
| UGT73C6 | 137 | 699 |
| UGT73E1 | 141 | 109 |
| UGT74D1 | 143 | 119 |
| UGT74G1 | 4 | 38967 |
| UGT75B1 | 145 | 1409 |
| UGT75L6 | 147 | 1208 |
| UGT76E12 | 153 | 161 |
| OleI | 177 | 1086 |
| UGT5 | 181 | 5547 |
| SA Gtase | 183 | 11088 |
| UDPG1 | 185 | 460 |
| UGT74F1 | 203 | 323 |
| UGT75D1 | 205 | 2465 |
| UGT84B2 | 207 | 31123 |
| CaUGT2 | 209 | 446 |
| UGT74F2-like UGT | 211 | 20552 |

TABLE 4
UGT Activity on steviol.

| | | Activity | |
|---|---|---|---|
| | SEQ ID | 13-SMG | 19-SMG |
| UGT Polypeptide | NO: | Production (AUC) | Production (AUC) |
| UGT73C1 | 127 | 9880 | 1235 |
| UGT73C3 | 133 | 1850 | 295 |
| UGT73C5 | 135 | 7100 | 2160 |
| UGT73C6 | 137 | 2255 | 4980 |
| UGT73C7 | 139 | 1570 | N/A |
| UGT73E1 | 141 | 2220 | 165 |
| UGT74G1 | 4 | N/A | 172485 |
| UGT75B1 | 145 | N/A | 230 |
| UGT75L6 | 147 | N/A | 4615 |
| UGT76E12 | 153 | 650 | N/A |
| UGT85C2 | 7 | 205575 | N/A |
| OleI | 177 | N/A | 540 |
| UGT5 | 181 | N/A | 1375 |
| SA Gtase | 183 | N/A | 10580 |
| UDPG1 | 185 | N/A | 4420 |

TABLE 5
UGT Activity on 13-SMG.

| | | Activity | |
|---|---|---|---|
| | SEQ ID | rubusoside | steviol-1,2-bioside |
| UGT Polypeptide | NO: | Production (AUC) | Production (AUC) |
| UGT73C1 | 127 | 550 | N/A |
| UGT73C6 | 137 | 1270 | N/A |
| UGT74G1 | 4 | 138650 | N/A |
| UGT85C2 | 7 | 865 | N/A |
| UGT91D2e-b | 13 | N/A | 1080 |
| EUGT11 | 16 | N/A | 10805 |
| SA Gtase | 183 | 4120 | N/A |
| UDPG1 | 185 | 2355 | N/A |
| UN32491 | 199 | N/A | 1065 |
| UN1671 | 201 | 1185 | N/A |
| UGT74F1 | 203 | 950 | N/A |
| UGT75D1 | 205 | 99885 | N/A |
| UGT84B2 | 207 | 1390 | N/A |
| UGT74F2-like UGT | 211 | 31415 | N/A |

TABLE 6
UGT Activity on rubusoside.

| | | Activity |
|---|---|---|
| | | 1,2-stevioside |
| | SEQ ID | Production |
| UGT Polypeptide | NO: | (AUC) |
| UGT73C6 | 137 | 385 |
| UGT91D2e-b | 13 | 4680 |
| CaUGT3 | 169 | 610 |
| EUGT11 | 16 | 1900 |

TABLE 7
UGT Activity on RebA.

| | | Activity |
|---|---|---|
| | SEQ ID | steviol + 5Glc (#24) |
| UGT Polypeptide | NO: | Production (AUC) |
| EUGT11 | 16 | 4950 |
| UN1671 | 201 | 52985 |

As shown in Tables 3-7, 19-O-glycosylation, 13-O-glycosylation, and glycosyl-group glycosylation activity by UGT polypeptides on several substrates was observed, resulting in the formation of glycosides of ent-kaurenoic acid and steviol.

TABLE 8
UGT Activity on 13-SMG and ent-kaurenoic acid.

| | SEQ ID | AUC rubusoside/ |
|---|---|---|
| UGT Polypeptide | NO: | AUC KA1.58 |
| UGT73C1 | 127 | 0.5 |
| UGT73C6 | 137 | 1.8 |
| UGT74G1 | 4 | 3.6 |
| SA Gtase | 183 | 0.4 |
| UDPG1 | 185 | 5.1 |
| UGT74F1 | 203 | 2.9 |
| UGT75D1 | 205 | 40.5 |
| UGT74F2-like UGT | 211 | 1.5 |

As shown in Table 8, UDPG1 (SEQ ID NO:185) and UGT75D1 (SEQ ID NO:205) produce relatively more rubusoside from 13-SMG than ent-kaurenoic acid+1Glc (#58) from ent-kaurenoic acid in vitro, compared to UGT74G1 (SEQ ID NO:4)

### Example 4

### Strain Engineering and Fermentation

SA Gtase (SEQ ID NO:182, SEQ ID NO:183) was expressed with a p416-GPD vector in a steviol glycoside-producing S. cerevisiae strain comprising one or more copies of a recombinant gene encoding a GGPPS polypeptide (SEQ ID NO:19, SEQ ID NO:20), a recombinant gene encoding a truncated CDPS polypeptide (SEQ ID NO:39, SEQ ID NO:40), a recombinant gene encoding an KS polypeptide (SEQ ID NO:51, SEQ ID NO:52), a recombinant gene encoding a KO polypeptide (SEQ ID NO:59, SEQ ID NO:60), a recombinant gene encoding an ATR2 polypeptide (SEQ ID NO:91, SEQ ID NO:92), a recombinant gene encoding an EUGT11 polypeptide (SEQ ID NO:14/SEQ ID NO:15, SEQ ID NO:16), a recombinant gene encoding an KAH polypeptide (SEQ ID NO:93, SEQ ID NO:94), a recombinant gene encoding a CPR8 polypeptide (SEQ ID NO:85, SEQ ID NO:86), a recombinant gene encoding an UGT85C2 polypeptide (SEQ ID NO:5/SEQ ID NO:6/SEQ ID NO:149, SEQ ID NO:7) or a UGT85C2 variant (or functional homolog) of SEQ ID NO:7, a recombinant gene encoding a UGT74G1 polypeptide (SEQ ID NO:3, SEQ ID NO:4) of a UGT74G1 variant (or functional homolog) of SEQ ID NO:4, a recombinant gene encoding a UGT76G1 polypeptide (SEQ ID NO:8, SEQ ID NO:9) or a UGT76G1 variant (or functional homolog) of SEQ ID NO:9, and a recombinant gene encoding a UGT91D2e polypeptide (SEQ ID NO:10, SEQ ID NO:11) and a UGT91D2e variant (or functional homolog) of SEQ ID NO:11 such as a UGT91D2e-b (SEQ ID NO:12, SEQ ID NO:13).

The strain was incubated in 1 mL synthetic complete (SC) uracil dropout media at 30° C. for five days, shaking at 400 rpm. 50 μL of each culture was transferred into 50 μL DMSO, incubated at 80° C. for 10 min, and centrifuged at 3220 g for 5 min. 15 μL of the resulting supernatant was then transferred to 105 μL 50% DMSO for LC-MS analysis, which was carried out according to Example 1. Normalized area-under-the-curve (AUC) values for LC-MS derived peaks corresponding to RebD and RebM were about 0.25 μM/OD₆₀₀ and 1.15 μM/OD₆₀₀, respectively. Ent-kaurenoic acid+2Glc (#7), ent-kaurenoic acid+3Glc (isomer 1), and ent-kaurenoic acid+3Glc (isomer 2) accumulated at levels of about 200 AUC/OD₆₀₀, 15 AUC/OD₆₀₀, and 1000 AUC/OD₆₀₀, respectively. 13-SMG, RebA, and Reb B accumulated at levels of about 4.8 μM/OD₆₀₀, 2.5 μM/OD₆₀₀, and 0.25 μM/OD₆₀₀, respectively. Steviol+4Glc (#26), steviol+6Glc (isomer 1), steviol+7Glc (isomer 2), and kaurenol+3Glc (isomer 1 and/or 2) accumulated at levels of about 200 AUC/OD₆₀₀, 15 AUC/OD₆₀₀, 75 AUC/OD₆₀₀, and 750 AUC/OD₆₀₀, respectively.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

TABLE 9
Sequences disclosed herein.

SEQ ID NO: 3
Artificial Sequence

| | |
|---|---|
| atggcagagc aacaaaagat caaaaagtca cctcacgtct tacttattcc atttcctctg | 60 |
| caaggacata tcaacccatt catacaattt gggaaaagat tgattagtaa gggtgtaaag | 120 |
| acaacactgg taaccactat ccacactttg aattctactc tgaaccactc aaatactact | 180 |
| actacaagta tagaaattca agctatatca gacggatgcg atgagggtgg ctttatgtct | 240 |
| gccggtgaat cttacttgga aacattcaag caagtgggat ccaagtctct ggccgatcta | 300 |
| atcaaaaagt tacagagtga aggcaccaca attgacgcca taatctacga ttctatgaca | 360 |
| gagtgggttt tagacgttgc tatcgaattt ggtattgatg gaggttcctt tttcacacaa | 420 |
| gcatgtgttg tgaattctct atactaccat gtgcataaag ggttaatctc tttaccattg | 480 |
| ggtgaaactg tttcagttcc aggttttcca gtgttacaac gttgggaaac cccattgatc | 540 |
| ttacaaaatc atgaacaaat acaatcacct tggtcccaga tgttgtttgg tcaattcgct | 600 |
| aacatcgatc aagcaagatg ggtctttact aattcattct ataagttaga ggaagaggta | 660 |
| attgaatgga ctaggaagat ctggaatttg aaagtcattg gtccaacatt gccatcaatg | 720 |
| tatttggaca aaagacttga tgatgataaa gataatggtt tcaatttgta caaggctaat | 780 |
| catcacgaat gtatgaattg gctggatgac aaaccaaagg aatcagttgt atatgttgct | 840 |
| ttcggctctc ttgttaaaca tggtccagaa caagttgagg agattacaag agcacttata | 900 |
| gactctgacg taaacttttt gtgggtcatt aagcacaaag aggaggggaa actgccagaa | 960 |
| aacctttctg aagtgataaa gaccggaaaa ggtctaatcg ttgcttggtg taaacaattg | 1020 |
| gatgttttag ctcatgaatc tgtaggctgt tttgtaacac attgcggatt caactctaca | 1080 |
| ctagaagcca tttccttagg cgtacctgtc gttgcaatgc ctcagttctc cgatcagaca | 1140 |
| accaacgcta aacttttgga cgaaatacta ggggtgggtg tcagagttaa agcagacgag | 1200 |
| aatggtatcg tcagaagagg gaacctagct tcatgtatca aaatgatcat ggaagaggaa | 1260 |
| agaggagtta tcataaggaa aaacgcagtt aagtggaagg atcttgcaaa ggttgccgtc | 1320 |
| catgaaggcg gctcttcaga taatgatatt gttgaatttg tgtccgaact aatcaaagcc | 1380 |
| taa | 1383 |

SEQ ID NO: 4
S. rebaudiana

| | |
|---|---|
| MAEQQKIKKS PHVLLIPFPL QGHINPFIQF GKRLISKGVK TTLVTTIHTL NSTLNHSNTT | 60 |
| TTSIEIQAIS DGCDEGGFMS AGESYLETFK QVGSKSLADL IKKLQSEGTT IDAIIYDSMT | 120 |
| EWVLDVAIEF GIDGGSFFTQ ACVVNSLYYH VHKGLISLPL GETVSVPGFP VLQRWETPLI | 180 |
| LQNHEQIQSP WSQMLFGQFA NIDQARWVFT NSFYKLEEEV IEWTRKIWNL KVIGPTLPSM | 240 |
| YLDKRLDDDK DNGFNLYKAN HHECMNWLDD KPKESVVYVA FGSLVKHGPE QVEEITRALI | 300 |
| DSDVNFLWVI KHKEEGKLPE NLSEVIKTGK GLIVAWCKQL DVLAHESVGC FVTHCGFNST | 360 |
| LEAISLGVPV VAMPQFSDQT TNAKLLDEIL GVGVRVKADE NGIVRRGNLA SCIKMIMEEE | 420 |
| RGVIIRKNAV KWKDLAKVAV HEGGSSDNDI VEFVSELIKA | 460 |

SEQ ID NO: 5
S. rebaudiana

| | |
|---|---|
| atggatgcaa tggctacaac tgagaagaaa ccacacgtca tcttcatacc atttccagca | 60 |
| caaagccaca ttaaagccat gctcaaacta gcacaacttc tccaccacaa aggactccag | 120 |
| ataaccttcg tcaacaccga cttcatccac aaccagtttc ttgaatcatc gggcccacat | 180 |
| tgtctagacg gtgcaccggg tttccggttc gaaaccattc cggatggtgt ttctcacagt | 240 |
| ccggaagcga gcatcccaat cagagaatca ctcttgagat ccattgaaac caacttcttg | 300 |
| gatcgtttca ttgatcttgt aaccaaactt ccggatcctc cgacttgtat tatctcagat | 360 |
| gggttcttgt cggttttcac aattgacgct gcaaaaaagc ttggaattcc ggtcatgatg | 420 |
| tattggacac ttgctgcctg tgggttcatg ggtttttacc atattcattc tctcattgag | 480 |
| aaaggatttg caccacttaa agatgcaagt tacttgacaa atgggtattt ggacaccgtc | 540 |
| attgattggg ttccgggaat ggaaggcatc cgtctcaagg atttcccgct ggactggagc | 600 |
| actgacctca atgacaaagt tttgatgttc actacggaag ctcctcaaag gtcacacaag | 660 |
| gtttcacatc atattttcca cacgttcgat gagttggagc ctagtattat aaaaactttg | 720 |
| tcattgaggt ataatcacat ttacaccatc ggcccactgc aattacttct tgatcaaata | 780 |
| cccgaagaga aaaagcaaac tggaattacg agtctccatg gatacagttt agtaaaagaa | 840 |
| gaaccagagt gtttccagtg gcttcagtct aaagaaccaa attccgtcgt ttatgtaaat | 900 |
| tttggaagta ctacagtaat gtctttagaa gacatgacgg aatttggttg gggacttgct | 960 |
| aatagcaacc attatttcct ttggatcatc cgatcaaact tggtgatagg ggaaaatgca | 1020 |
| gttttgcccc ctgaacttga ggaacatata aagaaaagag gctttattgc tagctggtgt | 1080 |
| tcacaagaaa aggtcttgaa gcacccttcg gttggagggt tcttgactca ttgtgggtgg | 1140 |
| ggatcgacca tcgagagctt gtctgctggg gtgccaatga tatgctggcc ttattcgtgg | 1200 |
| gaccagctga ccaactgtag gtatatatgc aaagaatggg aggttgggct cgagatggga | 1260 |
| accaaagtga aacgagatga agtcaagagg cttgtacaag agttgatggg agaaggaggt | 1320 |
| cacaaaatga ggaacaaggc taaagattgg aaagaaaagg ctcgcattgc aatagctcct | 1380 |
| aacggttcat cttctttgaa catagacaaa atggtcaagg aaatcaccgt gctagcaaga | 1440 |
| aactag | 1446 |

SEQ ID NO: 6
Artificial Sequence

| | |
|---|---|
| atggatgcaa tggcaactac tgagaaaaag cctcatgtga tcttcattcc atttcctgca | 60 |
| caatctcaca taaaggcaat gctaaagtta gcacaactat tacaccataa gggattacag | 120 |
| ataactttcg tgaataccga cttcatccat aatcaatttc tggaatctag tggccctcat | 180 |
| tgtttggacg gagccccagg gtttagattc gaaacaattc ctgacggtgt ttcacattcc | 240 |
| ccagaggcct ccatcccaat aagagagagt ttactgaggt caatagaaac caactttttg | 300 |
| gatcgtttca ttgacttggt cacaaaactt ccagacccac caacttgcat aatctctgat | 360 |
| ggctttctgt cagtgtttac tatcgacgct gccaaaaagt tgggtatccc agttatgatg | 420 |
| tactggactc ttgctgcatg cggtttcatg ggtttctatc acatccattc tcttatcgaa | 480 |
| aagggttttg ctccactgaa agatgcatca tacttaacca acggctacct ggatactgtt | 540 |
| attgactggg taccaggtat ggaaggtata agacttaaag attttccttt ggattggtct | 600 |
| acagacctta atgataaagt attgatgttt actacagaag ctccacaaag atctcataag | 660 |
| gtttcacatc atatctttca cacctttgat gaattggaac catcaatcat caaaaccttg | 720 |
| tctctaagat acaatcatat ctacactatt ggtccattac aattacttct agatcaaatt | 780 |
| cctgaagaga aaaagcaaac tggtattaca tccttacacg gctactcttt agtgaaagag | 840 |
| gaaccagaat gttttcaatg gctacaaagt aaagagccta attctgtggt ctacgtcaac | 900 |
| ttcggaagta caacagtcat gtccttggaa gatatgactg aatttggttg gggccttgct | 960 |
| aattcaaatc attactttct atggattatc aggtccaatt tggtaatagg ggaaaacgcc | 1020 |
| gtattacctc cagaattgga ggaacacatc aaaaagagag gtttcattgc ttcctggtgt | 1080 |
| tctcaggaaa aggtattgaa acatccttct gttggtggtt tccttactca ttgcggttgg | 1140 |
| ggctctacaa tcgaatcact aagtgcagga gttccaatga tttgttggcc atattcatgg | 1200 |
| gaccaactta caaattgtag gtatatctgt aaagagtggg aagttggatt agaaatggga | 1260 |
| acaaaggtta aacgtgatga agtgaaaaga ttggttcagg agttgatggg ggaaggtggc | 1320 |
| cacaagatga gaaacaaggc caaagattgg aaggaaaaag ccagaattgc tattgctcct | 1380 |
| aacgggtcat cctctctaaa cattgataag atggtcaaag agattacagt cttagccaga | 1440 |
| aactaa | 1446 |

SEQ ID NO: 7
S. rebaudiana

| | |
|---|---|
| MDAMATTEKK PHVIFIPFPA QSHIKAMLKL AQLLHHKGLQ ITFVNTDFIH NQFLESSGPH | 60 |
| CLDGAPGFRF ETIPDGVSHS PEASIPIRES LLRSIETNFL DRFIDLVTKL PDPPTCIISD | 120 |
| GFLSVFTIDA AKKLGIPVMM YWTLAACGFM GFYHIHSLIE KGFAPLKDAS YLTNGYLDTV | 180 |
| IDWVPGMEGI RLKDFPLDWS TDLNDKVLMF TTEAPQRSHK VSHHIFHTFD ELEPSIIKTL | 240 |
| SLRYNHIYTI GPLQLLLDQI PEEKKQTGIT SLHGYSLVKE EPECFQWLQS KEPNSVVYVN | 300 |
| FGSTTVMSLE DMTEFGWGLA NSNHYFLWII RSNLVIGENA VLPPELEEHI KKRGFIASWC | 360 |
| SQEKVLKHPS VGGFLTHCGW GSTIESLSAG VPMICWPYSW DQLTNCRYIC KEWEVGLEMG | 420 |
| TKVKRDEVKR LVQELMGEGG HKMRNKAKDW KEKARIAIAP NGSSSLNIDK MVKEITVLAR | 480 |
| N | 481 |

SEQ ID NO: 8
Artificial Sequence

| | |
|---|---|
| atggaaaaca agaccgaaac aacagttaga cgtaggcgta gaatcattct gtttccagta | 60 |
| ccttttcaag ggcacatcaa tccaatacta caactagcca acgttttgta ctctaaaggt | 120 |
| ttttctatta caatctttca caccaatttc aacaaaccaa aaacatccaa ttacccacat | 180 |
| ttcacattca gattcatact tgataatgat ccacaagatg aacgtatttc aaacttacct | 240 |
| acccacggtc ctttagctgg aatgagaatt ccaatcatca atgaacatgg tgccgatgag | 300 |
| cttagaagag aattagagtt acttatgttg gcatccgaag aggacgagga agtctcttgt | 360 |
| ctgattactg acgctctatg gtactttgcc caatctgtgg ctgatagttt gaatttgagg | 420 |
| agattggtac taatgacatc cagtctgttt aactttcacg ctcatgttag tttaccacaa | 480 |
| tttgacgaat tgggatactt ggaccctgat gacaagacta ggttagagga acaggcctct | 540 |
| ggttttccta tgttgaaagt caaagatatc aagtctgcct attctaattg gcaaatcttg | 600 |
| aaagagatct taggaaagat gatcaaacag acaaaggctt catctggagt gatttggaac | 660 |
| agtttcaaag agttagaaga gtctgaattg gagactgtaa tcagagaaat tccagcacct | 720 |
| tcattcctga taccattacc aaaacatttg actgcttcct cttcctcttt gttggatcat | 780 |
| gacagaacag tttttcaatg gttggaccaa caaccaccta gttctgtttt gtacgtgtca | 840 |
| tttggtagta cttctgaagt cgatgaaaag gacttccttg aaatcgcaag aggcttagtc | 900 |
| gatagtaagc agtcattcct ttgggtcgtg cgtccaggtt tcgtgaaagg ctcaacatgg | 960 |
| gtcgaaccac ttccagatgg ttttctaggc gaaagaggta gaatagtcaa atgggttcct | 1020 |
| caacaggaag ttttagctca tggcgctatt ggggcattct ggactcattc cggatggaat | 1080 |
| tcaactttag aatcagtatg cgaaggggta cctatgatct tttcagattt tggtcttgat | 1140 |
| caaccactga acgcaagata catgtctgat gttttgaaag tgggtgtata tctagaaaat | 1200 |
| ggctgggaaa ggggtgaaat agctaatgca ataagacgtg ttatggttga tgaagagggg | 1260 |
| gagtatatca gacaaaacgc aagagtgctg aagcaaaagg ccgacgtttc tctaatgaag | 1320 |
| ggaggctctt catacgaatc cttagaatct cttgtttcct acatttcatc actgtaa | 1377 |

SEQ ID NO: 9
S. rebaudiana

| | |
|---|---|
| MENKTETTVR RRRRIILFPV PFQGHINPIL QLANVLYSKG FSITIFHTNF NKPKTSNYPH | 60 |
| FTFRFILDND PQDERISNLP THGPLAGMRI PIINEHGADE LRRELELLML ASEEDEEVSC | 120 |
| LITDALWYFA QSVADSLNLR RLVLMTSSLF NFHAHVSLPQ FDELGYLDPD DKTRLEEQAS | 180 |
| GFPMLKVKDI KSAYSNWQIL KEILGKMIKQ TKASSGVIWN SFKELEESEL ETVIREIPAP | 240 |
| SFLIPLPKHL TASSSSLLDH DRTVFQWLDQ QPPSSVLYVS FGSTSEVDEK DFLEIARGLV | 300 |
| DSKQSFLWVV RPGFVKGSTW VEPLPDGFLG ERGRIVKWVP QQEVLAHGAI GAFWTHSGWN | 360 |
| STLESVCEGV PMIFSDFGLD QPLNARYMSD VLKVGVYLEN GWERGEIANA IRRVMVDEEG | 420 |
| EYIRQNARVL KQKADVSLMK GGSSYESLES LVSYISSL | 458 |

SEQ ID NO: 10
Artificial Sequence

| | |
|---|---|
| atggctacat ctgattctat tgttgatgac aggaagcagt tgcatgtggc tactttccct | 60 |
| tggcttgctt tcggtcatat actgccttac ctacaactat caaaactgat agctgaaaaa | 120 |
| ggacataaag tgtcattcct ttcaacaact agaaacattc aaagattatc ttcccacata | 180 |
| tcaccattga ttaacgtcgt tcaattgaca cttccaagag tacaggaatt accagaagat | 240 |
| gctgaagcta caacagatgt gcatcctgaa gatatccctt acttgaaaaa ggcatccgat | 300 |
| ggattacagc ctgaggtcac tagattcctt gagcaacaca gtccagattg gatcatatac | 360 |
| gactacactc actattggtt gccttcaatt gcagcatcac taggcatttc tagggcacat | 420 |
| ttcagtgtaa ccacaccttg ggccattgct tacatgggtc catccgctga tgctatgatt | 480 |
| aacggcagtg atggtagaac taccgttgaa gatttgacaa ccccaccaaa gtggtttcca | 540 |
| tttccaacta aagtctgttg gagaaaacac gacttagcaa gactggttcc atacaaggca | 600 |
| ccaggaatct cagacggcta tagaatgggt ttagtcctta aagggtctga ctgcctattg | 660 |
| tctaagtgtt accatgagtt tgggacacaa tggctaccac ttttggaaac attacaccaa | 720 |
| gttcctgtcg taccagttgg tctattacct ccagaaatcc ctggtgatga gaaggacgag | 780 |
| acttgggttt caatcaaaaa gtggttagac gggaagcaaa aaggctcagt ggtatatgtg | 840 |
| gcactgggtt ccgaagtttt agtatctcaa acagaagttg tggaacttgc cttaggtttg | 900 |
| gaactatctg gattgccatt tgtctgggcc tacagaaaac caaaaggccc tgcaaagtcc | 960 |
| gattcagttg aattgccaga cggctttgtc gagagaacta gagatagagg gttggtatgg | 1020 |
| acttcatggg ctccacaatt gagaatcctg agtcacgaat ctgtgtgcgg tttcctaaca | 1080 |
| cattgtggtt ctggttctat agttgaagga ctgatgtttg gtcatccact tatcatgttg | 1140 |
| ccaatctttg gtgaccagcc tttgaatgca cgtctgttag aagataaaca agttggaatt | 1200 |
| gaaatcccac gtaatgagga agatggatgt ttaaccaagg agtctgtggc cagatcatta | 1260 |
| cgttccgttg tcgttgaaaa ggaaggcgaa atctacaagg ccaatgcccg tgaactttca | 1320 |
| aagatctaca atgacacaaa agtagagaag gaatatgttt ctcaatttgt agattaccta | 1380 |
| gagaaaaacg ctagagccgt agctattgat catgaatcct aa | 1422 |

SEQ ID NO: 11
S. rebaudiana

| | |
|---|---|
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG LVLKGSDCLL SKCYHEFGTQ WLPLLETLHQ | 240 |
| VPVVPVGLLP PEIPGDEKDE TWVSIKKWLD GKQKGSVVYV ALGSEVLVSQ TEVVELALGL | 300 |
| ELSGLPFVWA YRKPKGPAKS DSVELPDGFV ERTRDRGLVW TSWAPQLRIL SHESVCGFLT | 360 |
| HCGSGSIVEG LMFGHPLIML PIFGDQPLNA RLLEDKQVGI EIPRNEEDGC LTKESVARSL | 420 |
| RSVVVEKEGE IYKANARELS KIYNDTKVEK EYVSQFVDYL EKNARAVAID HES | 473 |

SEQ ID NO: 12
Artificial Sequence

| | |
|---|---|
| atggctactt ctgattccat cgttgacgat agaaagcaat tgcatgttgc tacttttcca | 60 |
| tggttggctt tcggtcatat tttgccatac ttgcaattgt ccaagttgat tgctgaaaag | 120 |
| ggtcacaagg tttcattctt gtctaccacc agaaacatcc aaagattgtc ctctcatatc | 180 |
| tccccattga tcaacgttgt tcaattgact ttgccaagag tccaagaatt gccagaagat | 240 |
| gctgaagcta ctactgatgt tcatccagaa gatatccctt acttgaaaaa ggcttccgat | 300 |
| ggtttacaac cagaagttac tagattcttg gaacaacatt ccccagattg gatcatctac | 360 |
| gattatactc attactggtt gccatccatt gctgcttcat tgggtatttc tagagcccat | 420 |
| ttctctgtta ctactccatg ggctattgct tatatgggtc catctgctga tgctatgatt | 480 |
| aacggttctg atggtagaac taccgttgaa gatttgacta ctccaccaaa gtggtttcca | 540 |
| tttccaacaa aagtctgttg gagaaaacac gatttggcta gattggttcc atacaaagct | 600 |
| ccaggtattt ctgatggtta cagaatgggt atggttttga aaggttccga ttgcttgttg | 660 |
| tctaagtgct atcatgaatt cggtactcaa tggttgcctt tgttggaaac attgcatcaa | 720 |
| gttccagttg ttccagtagg tttgttgcca ccagaaattc caggtgacga aaaagacgaa | 780 |
| acttgggttt ccatcaaaaa gtggttggat ggtaagcaaa agggttctgt tgtttatgtt | 840 |
| gctttgggtt ccgaagcttt ggtttctcaa accgaagttg ttgaattggc tttgggtttg | 900 |
| gaattgtctg gtttgccatt tgtttgggct tacagaaaac ctaaaggtcc agctaagtct | 960 |
| gattctgttg aattgccaga tggtttcgtt gaaagaacta gagatagagg tttggtttgg | 1020 |
| acttcttggg ctccacaatt gagaattttg tctcatgaat ccgtctgtgg tttcttgact | 1080 |
| cattgtggtt ctggttctat cgttgaaggt ttgatgtttg gtcacccatt gattatgttg | 1140 |
| ccaatctttg gtgaccaacc attgaacgct agattattgg aagataagca agtcggtatc | 1200 |
| gaaatcccaa gaaatgaaga agatggttgc ttgaccaaag aatctgttgc tagatctttg | 1260 |
| agatccgttg tcgttgaaaa agaaggtgaa atctacaagg ctaacgctag agaattgtcc | 1320 |
| aagatctaca acgataccaa ggtcgaaaaa gaatacgttt cccaattcgt tgactacttg | 1380 |
| gaaaagaatg ctagagctgt tgccattgat catgaatctt ga | 1422 |

SEQ ID NO: 13
Artificial Sequence

| | |
|---|---|
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG MVLKGSDCLL SKCYHEFGTQ WLPLLETLHQ | 240 |
| VPVVPVGLLP PEIPGDEKDE TWVSIKKWLD GKQKGSVVYV ALGSEALVSQ TEVVELALGL | 300 |
| ELSGLPFVWA YRKPKGPAKS DSVELPDGFV ERTRDRGLVW TSWAPQLRIL SHESVCGFLT | 360 |
| HCGSGSIVEG LMFGHPLIML PIFGDQPLNA RLLEDKQVGI EIPRNEEDGC LTKESVARSL | 420 |
| RSVVVEKEGE IYKANARELS KIYNDTKVEK EYVSQFVDYL EKNARAVAID HES | 473 |

SEQ ID NO: 14
Oryza sativa

| | |
|---|---|
| atggactccg gctactcctc ctcctacgcc gccgccgccg ggatgcacgt cgtgatctgc | 60 |
| ccgtggctcg ccttcggcca cctgctcccg tgcctcgacc tcgcccagcg cctcgcgtcg | 120 |
| cggggccacc gcgtgtcgtt cgtctccacg ccgcggaaca tatcccgcct cccgccggtg | 180 |
| cgccccgcgc tcgcgccgct cgtcgccttc gtggcgctgc cgctcccgcg cgtcgagggg | 240 |
| ctccccgacg gcgccgagtc caccaacgac gtcccccacg acaggccgga catggtcgag | 300 |
| ctccaccgga gggccttcga cgggctcgcc gcgcccttct cggagttctt gggcaccgcg | 360 |
| tgcgccgact gggtcatcgt cgacgtcttc caccactggg ccgcagccgc cgctctcgag | 420 |
| cacaaggtgc catgtgcaat gatgttgttg ggctctgcac atatgatcgc ttccatagca | 480 |
| gacagacggc tcgagcgcgc ggagacagag tcgcctgcgg ctgccgggca gggacgccca | 540 |
| gcggcggcgc caacgttcga ggtggcgagg atgaagttga tacgaaccaa aggctcatcg | 600 |
| ggaatgtccc tcgccgagcg cttctccttg acgctctcga ggagcagcct cgtcgtcggg | 660 |
| cggagctgcg tggagttcga gccggagacc gtcccgctcc tgtcgacgct ccgcggtaag | 720 |
| cctattacct tccttggcct tatgccgccg ttgcatgaag gccgccgcga ggacggcgag | 780 |
| gatgccaccg tccgctggct cgacgcgcag ccggccaagt ccgtcgtgta cgtcgcgcta | 840 |
| ggcagcgagg tgccactggg agtggagaag gtccacgagc tcgcgctcgg gctggagctc | 900 |
| gccgggacgc gcttcctctg ggctcttagg aagcccactg gcgtctccga cgccgacctc | 960 |
| ctccccgccg gcttcgagga gcgcacgcgc ggccgcggcg tcgtggcgac gagatgggtt | 1020 |
| cctcagatga gcatactggc gcacgccgcc gtgggcgcgt tcctgaccca ctgcggctgg | 1080 |
| aactcgacca tcgaggggct catgttcggc cacccgctta tcatgctgcc gatcttcggc | 1140 |
| gaccagggac cgaacgcgcg gctaatcgag gcgaagaacg ccggattgca ggtggcaaga | 1200 |
| aacgacggcg atggatcgtt cgaccgagaa ggcgtcgcgg cggcgattcg tgcagtcgcg | 1260 |
| gtggaggaag aaagcagcaa agtgtttcaa gccaaagcca agaagctgca ggagatcgtc | 1320 |
| gcggacatgg cctgccatga gaggtacatc gacggattca ttcagcaatt gagatcttac | 1380 |
| aaggattga | 1389 |

SEQ ID NO: 15
Artificial Sequence

| | |
|---|---|
| atggatagtg gctactcctc atcttatgct gctgccgctg gtatgcacgt tgtgatctgc | 60 |
| ccttggttgg cctttggtca cctgttacca tgtctggatt tagcccaaag actggcctca | 120 |
| agaggccata gagtatcatt tgtgtctact cctagaaata tctctcgttt accaccagtc | 180 |
| agacctgctc tagctcctct agttgcattc gttgctcttc cacttccaag agtagaagga | 240 |
| ttgccagacg gcgctgaatc tactaatgac gtaccacatg atagacctga catggtcgaa | 300 |
| ttgcatagaa gagcctttga tggattggca gctccatttt ctgagttcct gggcacagca | 360 |
| tgtgcagact gggttatagt cgatgtattt catcactggg ctgctgcagc cgcattggaa | 420 |
| cataaggtgc cttgtgctat gatgttgtta gggtcagcac acatgatcgc atccatagct | 480 |
| gatagaagat tggaaagagc tgaaacagaa tccccagccg cagcaggaca aggtaggcca | 540 |
| gctgccgccc caacctttga agtggctaga atgaaattga ttcgtactaa aggtagttca | 600 |
| gggatgagtc ttgctgaaag gttttctctg acattatcta gatcatcatt agttgtaggt | 660 |
| agatcctgcg tcgagttcga acctgaaaca gtacctttac tatctacttt gagaggcaaa | 720 |
| cctattactt tccttggtct aatgcctcca ttacatgaag gaaggagaga agatggtgaa | 780 |
| gatgctactg ttaggtggtt agatgcccaa cctgctaagt ctgttgttta cgttgcattg | 840 |
| ggttctgagg taccactagg ggtggaaaag gtgcatgaat tagcattagg acttgagctg | 900 |
| gccggaacaa gattcctttg ggctttgaga aaaccaaccg gtgtttctga cgccgacttg | 960 |
| ctaccagctg ggttcgaaga gagaacaaga ggccgtggtg tcgttgctac tagatgggtc | 1020 |
| ccacaaatga gtattctagc tcatgcagct gtaggggcct ttctaaccca ttgcggttgg | 1080 |
| aactcaacaa tagaaggact gatgtttggt catccactta ttatgttacc aatctttggc | 1140 |
| gatcagggac ctaacgcaag attgattgag gcaaagaacg caggtctgca ggttgcacgt | 1200 |
| aatgatggtg atggttcctt tgatagagaa ggcgttgcag ctgccatcag agcagtcgcc | 1260 |
| gttgaggaag agtcatctaa agttttccaa gctaaggcca aaaaattaca agagattgtg | 1320 |
| gctgacatgg cttgtcacga aagatacatc gatggtttca tccaacaatt gagaagttat | 1380 |
| aaagactaa | 1389 |

SEQ ID NO: 16
Oryza sativa

| | |
|---|---|
| MDSGYSSSYA AAAGMHVVIC PWLAFGHLLP CLDLAQRLAS RGHRVSFVST PRNISRLPPV | 60 |
| RPALAPLVAF VALPLPRVEG LPDGAESTND VPHDRPDMVE LHRRAFDGLA APFSEFLGTA | 120 |
| CADWVIVDVF HHWAAAAALE HKVPCAMMLL GSAHMIASIA DRRLERAETE SPAAAGQGRP | 180 |
| AAAPTFEVAR MKLIRTKGSS GMSLAERFSL TLSRSSLVVG RSCVEFEPET VPLLSTLRGK | 240 |
| PITFLGLMPP LHEGRREDGE DATVRWLDAQ PAKSVVYVAL GSEVPLGVEK VHELALGLEL | 300 |
| AGTRFLWALR KPTGVSDADL LPAGFEERTR GRGVVATRWV PQMSILAHAA VGAFLTHCGW | 360 |
| NSTIEGLMFG HPLIMLPIFG DQGPNARLIE AKNAGLQVAR NDGDGSFDRE GVAAAIRAVA | 420 |
| VEEESSKVFQ AKAKKLQEIV ADMACHERYI DGFIQQLRSY KD | 462 |

SEQ ID NO: 17
Artificial Sequence

| | |
|---|---|
| MDSGYSSSYA AAAGMHVVIC PWLAFGHLLP CLDLAQRLAS RGHRVSFVST PRNISRLPPV | 60 |
| RPALAPLVAF VALPLPRVEG LPDGAESTND VPHDRPDMVE LHRRAFDGLA APFSEFLGTA | 120 |
| CADWVIVDVF HHWAAAAALE HKVPCAMMLL GSAHMIASIA DRRLERAETE SPAAAGQGRP | 180 |
| AAAPTFEVAR MKLIRTKGSS GMSLAERFSL TLSRSSLVVG RSCVEFEPET VPLLSTLRGK | 240 |
| PITFLGLLPP EIPGDEKDET WVSIKKWLDG KQKGSVVYVA LGSEALVSQT EVVELALGLE | 300 |
| LSGLPFVWAY RKPKGPAKSD SVELPDGFVE RTRDRGLVWT SWAPQLRILS HESVCGFLTH | 360 |
| CGSGSIVEGL MFGHPLIMLP IFGDQPLNAR LLEDKQVGIE IARNDGDGSF DREGVAAAIR | 420 |
| AVAVEEESSK VFQAKAKKLQ EIVADMACHE RYIDGFIQQL RSYKD | 465 |

SEQ ID NO: 18
Artificial Sequence

| | |
|---|---|
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG MVLKGSDCLL SKCYHEFGTQ WLPLLETLHQ | 240 |
| VPVVPVGLMP PLHEGRREDG EDATVRWLDA QPAKSVVYVA LGSEVPLGVE KVHELALGLE | 300 |
| LAGTRFLWAL RKPTGVSDAD LLPAGFEERT RGRGVVATRW VPQMSILAHA AVGAFLTHCG | 360 |
| WNSTIEGLMF GHPLIMLPIF GDQGPNARLI EAKNAGLQVP RNEEDGCLTK ESVARSLRSV | 420 |
| VVEKEGEIYK ANARELSKIY NDTKVEKEYV SQFVDYLEKN ARAVAIDHES | 470 |

SEQ ID NO: 19
Stevia rebaudiana

| | |
|---|---|
| atggctttgg taaacccaac cgctcttttc tatggtacct ctatcagaac aagacctaca | 60 |
| aacttactaa atccaactca aaagctaaga ccagtttcat catcttcctt accttctttc | 120 |
| tcatcagtta gtgcgattct tactgaaaaa catcaatcta atccttctga gaacaacaat | 180 |
| ttgcaaactc atctagaaac tcctttcaac tttgatagtt atatgttgga aaaagtcaac | 240 |
| atggttaacg aggcgcttga tgcatctgtc ccactaaaag acccaatcaa aatccatgaa | 300 |
| tccatgagat actctttatt ggcaggcggt aagagaatca gaccaatgat gtgtattgca | 360 |
| gcctgcgaaa tagtcggagg taatatcctt aacgccatgc cagccgcatg tgccgtggaa | 420 |
| atgattcata ctatgtcttt ggtgcatgac gatcttccat gtatggataa tgatgacttc | 480 |
| agaagaggta aacctatttc acacaaggtc tacggggagg aaatggcagt attgaccggc | 540 |
| gatgctttac taagtttatc tttcgaacat atagctactg ctacaaaggg tgtatcaaag | 600 |
| gatagaatcg tcagagctat aggggagttg gcccgttcag ttggctccga aggtttagtg | 660 |
| gctggacaag ttgtagatat cttgtcagag ggtgctgatg ttggattaga tcacctagaa | 720 |
| tacattcaca tccacaaaac agcaatgttg cttgagtcct cagtagttat tggcgctatc | 780 |
| atgggaggag gatctgatca gcagatcgaa aagttgagaa aattcgctag atctattggt | 840 |
| ctactattcc aagttgtgga tgacattttg gatgttacaa aatctaccga agagttgggg | 900 |
| aaaacagctg gtaaggattt gttgacagat aagacaactt acccaaagtt gttaggtata | 960 |
| gaaaagtcca gagaatttgc cgaaaaactt aacaaggaag cacaagagca attaagtggc | 1020 |
| tttgatagac gtaaggcagc tcctttgatc gcgttagcca actacaatgc gtaccgtcaa | 1080 |
| aattga | 1086 |

SEQ ID NO: 20
Stevia rebaudiana

| | |
|---|---|
| MALVNPTALF YGTSIRTRPT NLLNPTQKLR PVSSSSLPSF SSVSAILTEK HQSNPSENNN | 60 |
| LQTHLETPFN FDSYMLEKVN MVNEALDASV PLKDPIKIHE SMRYSLLAGG KRIRPMMCIA | 120 |
| ACEIVGGNIL NAMPAACAVE MIHTMSLVHD DLPCMDNDDF RRGKPISHKV YGEEMAVLTG | 180 |
| DALLSLSFEH IATATKGVSK DRIVRAIGEL ARSVGSEGLV AGQVVDILSE GADVGLDHLE | 240 |
| YIHIHKTAML LESSVVIGAI MGGGSDQQIE KLRKFARSIG LLFQVVDDIL DVTKSTEELG | 300 |
| KTAGKDLLTD KTTYPKLLGI EKSREFAEKL NKEAQEQLSG FDRRKAAPLI ALANYNAYRQ | 360 |
| N | 361 |

SEQ ID NO: 21
Artificial Sequence

| | |
|---|---|
| atggctgagc aacaaatatc taacttgctg tctatgtttg atgcttcaca tgctagtcag | 60 |
| aaattagaaa ttactgtcca aatgatggac acataccatt acagagaaac gcctccagat | 120 |
| tcctcatctt ctgaaggcgg ttcattgtct agatacgacg agagaagagt ctctttgcct | 180 |
| ctcagtcata atgctgcctc tccagatatt gtatcacaac tatgtttttc cactgcaatg | 240 |
| tcttcagagt tgaatcacag atggaaatct caaagattaa aggtggccga ttctccttac | 300 |
| aactatatcc taacattacc atcaaaagga attagaggtg cctttatcga ttccctgaac | 360 |
| gtatggttgg aggttccaga ggatgaaaca tcagtcatca aggaagttat tggtatgctc | 420 |
| cacaactctt cattaatcat tgatgacttc caagataatt ctccacttag aagaggaaag | 480 |
| ccatctaccc atacagtctt cggccctgcc caggctatca atactgctac ttacgttata | 540 |
| gttaaagcaa tcgaaaagat acaagacata gtgggacacg atgcattggc agatgttacg | 600 |
| ggtactatta caactatttt ccaaggtcag gccatggact tgtggtggac agcaaatgca | 660 |
| atcgttccat caatacagga atacttactt atggtaaacg ataaaaccgg tgctctcttt | 720 |
| agactgagtt tggagttgtt agctctgaat tccgaagcca gtatttctga ctctgcttta | 780 |
| gaaagtttat ctagtgctgt ttccttgcta ggtcaatact tccaaatcag agacgactat | 840 |
| atgaacttga tcgataacaa gtatacagat cagaaaggct tctgcgaaga tcttgatgaa | 900 |
| ggcaagtact cactaacact tattcatgcc ctccaaactg attcatccga tctactgacc | 960 |
| aacatccttt caatgagaag agtgcaagga aagttaacgg cacaaaagag atgttggttc | 1020 |
| tggaaatga | 1029 |

SEQ ID NO: 22
Gibberella fujikuroi

| | |
|---|---|
| MAEQQISNLL SMFDASHASQ KLEITVQMMD TYHYRETPPD SSSSEGGSLS RYDERRVSLP | 60 |
| LSHNAASPDI VSQLCFSTAM SSELNHRWKS QRLKVADSPY NYILTLPSKG IRGAFIDSLN | 120 |
| VWLEVPEDET SVIKEVIGML HNSSLIIDDF QDNSPLRRGK PSTHTVFGPA QAINTATYVI | 180 |
| VKAIEKIQDI VGHDALADVT GTITTIFQGQ AMDLWWTANA IVPSIQEYLL MVNDKTGALF | 240 |
| RLSLELLALN SEASISDSAL ESLSSAVSLL GQYFQIRDDY MNLIDNKYTD QKGFCEDLDE | 300 |
| GKYSLTLIHA LQTDSSDLLT NILSMRRVQG KLTAQKRCWF WK | 342 |

SEQ ID NO: 23
Artificial Sequence

| | |
|---|---|
| atggaaaaga ctaaggagaa agcagaacgt atcttgctgg agccatacag atacttatta | 60 |
| caactaccag gaaagcaagt ccgttctaaa ctatcacaag cgttcaatca ctggttaaaa | 120 |
| gttcctgaag ataagttaca aatcattatt gaagtcacag aaatgctaca caatgcttct | 180 |
| ttactgatcg atgatataga ggattcttcc aaactgagaa gaggttttcc tgtcgctcat | 240 |
| tccatatacg gggtaccaag tgtaatcaac tcagctaatt acgtctactt cttgggattg | 300 |
| gaaaaagtat tgacattaga tcatccagac gctgtaaagc tattcaccag acaacttctt | 360 |
| gaattgcatc aaggtcaagg tttggatatc tattggagag acacttatac ttgcccaaca | 420 |
| gaagaggagt acaaagcaat ggttctacaa aagactggcg gtttgttcgg acttgccgtt | 480 |
| ggtctgatgc aacttttctc tgattacaag gaggacttaa agcctctgtt ggataccttg | 540 |
| ggcttgtttt tccagattag agatgactac gctaacttac attcaaagga atattcagaa | 600 |
| aacaaatcat tctgtgaaga tttgactgaa gggaagttta gttttccaac aatccacgcc | 660 |
| atttggtcaa gaccagaatc tactcaagtg caaaacattc tgcgtcagag aacagagaat | 720 |
| attgacatca aaaagtattg tgttcagtac ttggaagatg ttggttcttt tgcttacaca | 780 |
| agacatacac ttagagaatt agaggcaaaa gcatacaagc aaatagaagc ctgtggaggc | 840 |
| aatccttctc tagtggcatt ggttaaacat ttgtccaaaa tgttcaccga ggaaaacaag | 900 |
| taa | 903 |

SEQ ID NO: 24
Mus musculus

| | |
|---|---|
| MEKTKEKAER ILLEPYRYLL QLPGKQVRSK LSQAFNHWLK VPEDKLQIII EVTEMLHNAS | 60 |
| LLIDDIEDSS KLRRGFPVAH SIYGVPSVIN SANYVYFLGL EKVLTLDHPD AVKLFTRQLL | 120 |
| ELHQGQGLDI YWRDTYTCPT EEEYKAMVLQ KTGGLFGLAV GLMQLFSDYK EDLKPLLDTL | 180 |
| GLFFQIRDDY ANLHSKEYSE NKSFCEDLTE GKFSFPTIHA IWSRPESTQV QNILRQRTEN | 240 |
| IDIKKYCVQY LEDVGSFAYT RHTLRELEAK AYKQIEACGG NPSLVALVKH LSKMFTEENK | 300 |

SEQ ID NO: 25
Artificial Sequence

| | |
|---|---|
| atggcaagat tctattttct taacgcacta ttgatggtta tctcattaca atcaactaca | 60 |
| gccttcactc cagctaaact tgcttatcca acaacaacaa cagctctaaa tgtcgcctcc | 120 |
| gccgaaactt ctttcagtct agatgaatac ttggcctcta agataggacc tatagagtct | 180 |
| gccttggaag catcagtcaa atccagaatt ccacagaccg ataagatctg cgaatctatg | 240 |
| gcctactctt tgatggcagg aggcaagaga attagaccag tgttgtgtat cgctgcatgt | 300 |
| gagatgttcg gtggatccca agatgtcgct atgcctactg ctgtggcatt agaaatgata | 360 |
| cacacaatgt ctttgattca tgatgatttg ccatccatgg ataacgatga cttgagaaga | 420 |
| ggtaaaccaa caaaccatgt cgttttcggc gaagatgtag ctattcttgc aggtgactct | 480 |
| ttattgtcaa cttccttcga gcacgtcgct agagaaacaa aaggagtgtc agcagaaaag | 540 |
| atcgtggatg ttatcgctag attaggcaaa tctgttggtg ccgagggcct tgctggcggt | 600 |
| caagttatgg acttagaatg tgaagctaaa ccaggtacca cattagacga cttgaaatgg | 660 |
| attcatatcc ataaaaccgc tacattgtta caagttgctg tagcttctgg tgcagttcta | 720 |
| ggtggtgcaa ctcctgaaga ggttgctgca tgcgagttgt ttgctatgaa tataggtctt | 780 |
| gcctttcaag ttgccgacga tatccttgat gtaaccgctt catcagaaga tttgggtaaa | 840 |
| actgcaggca aagatgaagc tactgataag acaacttacc caaagttatt aggattagaa | 900 |
| gagagtaagg catacgcaag acaactaatc gatgaagcca aggaaagttt ggctcctttt | 960 |
| ggagatagag ctgccccttt attggccatt gcagatttca ttattgatag aaagaattga | 1020 |

SEQ ID NO: 26
Thalassiosira pseudonana

| | |
|---|---|
| MARFYFLNAL LMVISLQSTT AFTPAKLAYP TTTTALNVAS AETSFSLDEY LASKIGPIES | 60 |
| ALEASVKSRI PQTDKICESM AYSLMAGGKR IRPVLCIAAC EMFGGSQDVA MPTAVALEMI | 120 |
| HTMSLIHDDL PSMDNDDLRR GKPTNHVVFG EDVAILAGDS LLSTSFEHVA RETKGVSAEK | 180 |
| IVDVIARLGK SVGAEGLAGG QVMDLECEAK PGTTLDDLKW IHIHKTATLL QVAVASGAVL | 240 |
| GGATPEEVAA CELFAMNIGL AFQVADDILD VTASSEDLGK TAGKDEATDK TTYPKLLGLE | 300 |
| ESKAYARQLI DEAKESLAPF GDRAAPLLAI ADFIIDRKN | 339 |

SEQ ID NO: 27
Artificial Sequence

| | |
|---|---|
| atgcacttag caccacgtag agtccctaga ggtagaagat caccacctga cagagttcct | 60 |
| gaaagacaag gtgccttggg tagaagacgt ggagctggct ctactggctg tgcccgtgct | 120 |
| gctgctggtg ttcaccgtag aagaggagga ggcgaggctg atccatcagc tgctgtgcat | 180 |
| agaggctggc aagccggtgg tggcaccggt ttgcctgatg aggtggtgtc taccgcagcc | 240 |
| gccttagaaa tgtttcatgc ttttgcttta atccatgatg atatcatgga tgatagtgca | 300 |
| actagaagag gctccccaac tgttcacaga gccctagctg atcgtttagg cgctgctctg | 360 |
| gacccagatc aggccggtca actaggagtt tctactgcta tcttggttgg agatctggct | 420 |
| ttgacatggt ccgatgaatt gttatacgct ccattgactc cacatagact ggcagcagta | 480 |
| ctaccattgg taacagctat gagagctgaa accgttcatg gccaatatct tgatataact | 540 |
| agtgctagaa gacctgggac cgatacttct cttgcattga gaatagccag atataagaca | 600 |
| gcagcttaca caatggaacg tccactgcac attggtgcag ccctggctgg ggcaagacca | 660 |
| gaactattag cagggctttc agcatacgcc ttgccagctg gagaagcctt ccaattggca | 720 |
| gatgacctgc taggcgtctt cggtgatcca agacgtacag ggaaacctga cctagatgat | 780 |
| cttagaggtg gaaagcatac tgtcttagtc gccttggcaa gagaacatgc cactccagaa | 840 |
| cagagacaca cattggatac attattgggt acaccaggtc ttgatagaca aggcgcttca | 900 |
| agactaagat gcgtattggt agcaactggt gcaagagccg aagccgaaag acttattaca | 960 |
| gagagaagag atcaagcatt aactgcattg aacgcattaa cactgccacc tcctttagct | 1020 |
| gaggcattag caagattgac attagggtct acagctcatc ctgcctaa | 1068 |

SEQ ID NO: 28
Streptomyces clavuligerus

| | |
|---|---|
| MHLAPRRVPR GRRSPPDRVP ERQGALGRRR GAGSTGCARA AAGVHRRRGG GEADPSAAVH | 60 |
| RGWQAGGGTG LPDEVVSTAA ALEMFHAFAL IHDDIMDDSA TRRGSPTVHR ALADRLGAAL | 120 |
| DPDQAGQLGV STAILVGDLA LTWSDELLYA PLTPHRLAAV LPLVTAMRAE TVHGQYLDIT | 180 |
| SARRPGTDTS LALRIARYKT AAYTMERPLH IGAALAGARP ELLAGLSAYA LPAGEAFQLA | 240 |
| DDLLGVFGDP RRTGKPDLDD LRGGKHTVLV ALAREHATPE QRHTLDTLLG TPGLDRQGAS | 300 |
| RLRCVLVATG ARAEAERLIT ERRDQALTAL NALTLPPPLA EALARLTLGS TAHPA | 355 |

SEQ ID NO: 29
Artificial Sequence

| | |
|---|---|
| atgtcatatt tcgataacta cttcaatgag atagttaatt ccgtgaacga catcattaag | 60 |
| tcttacatct ctggcgacgt accaaaacta tacgaagcct cctaccattt gtttacatca | 120 |
| ggaggaaaga gactaagacc attgatcctt acaatttctt ctgatctttt cggtggacag | 180 |
| agagaaagag catactatgc tggcgcagca atcgaagttt tgcacacatt cactttggtt | 240 |
| cacgatgata tcatggatca agataacatt cgtagaggtc ttcctactgt acatgtcaag | 300 |
| tatggcctac ctttggccat tttagctggt gacttattgc atgcaaaagc ctttcaattg | 360 |
| ttgactcagg cattgagagg tctaccatct gaaactatca tcaaggcgtt tgatatcttt | 420 |
| acaagatcta tcattatcat atcagaaggt caagctgtcg atatggaatt cgaagataga | 480 |
| attgatatca aggaacaaga gtatttggat atgatatctc gtaaaaccgc tgccttattc | 540 |
| tcagcttctt cttccattgg ggcgttgata gctggagcta atgataacga tgtgagatta | 600 |
| atgtccgatt tcggtacaaa tcttgggatc gcatttcaaa ttgtagatga tatacttggt | 660 |
| ttaacagctg atgaaaaaga gctaggaaaa cctgttttca gtgatatcag agaaggtaaa | 720 |
| aagaccatat tagtcattaa gactttagaa ttgtgtaagg aagacgagaa aaagattgtg | 780 |
| ttaaaagcgc taggcaacaa gtcagcatca aaggaagagt tgatgagttc tgctgacata | 840 |
| atcaaaaagt actcattgga ttacgcctac aacttagctg agaaatacta caaaaacgcc | 900 |
| atcgattctc taaatcaagt ttcaagtaaa agtgatattc cagggaaggc attgaaatat | 960 |
| cttgctgaat tcaccatcag aagacgtaag taa | 993 |

SEQ ID NO: 30
Sulfolobus acidocaldarius

| | |
|---|---|
| MSYFDNYFNE IVNSVNDIIK SYISGDVPKL YEASYHLFTS GGKRLRPLIL TISSDLFGGQ | 60 |
| RERAYYAGAA IEVLHTFTLV HDDIMDQDNI RRGLPTVHVK YGLPLAILAG DLLHAKAFQL | 120 |
| LTQALRGLPS ETIIKAFDIF TRSIIIISEG QAVDMEFEDR IDIKEQEYLD MISRKTAALF | 180 |
| SASSSIGALI AGANDNDVRL MSDFGTNLGI AFQIVDDILG LTADEKELGK PVFSDIREGK | 240 |
| KTILVIKTLE LCKEDEKKIV LKALGNKSAS KEELMSSADI IKKYSLDYAY NLAEKYYKNA | 300 |
| IDSLNQVSSK SDIPGKALKY LAEFTIRRRK | 330 |

SEQ ID NO: 31
Artificial Sequence

| | |
|---|---|
| atggtcgcac aaactttcaa cctggatacc tacttatccc aaagacaaca acaagttgaa | 60 |
| gaggccctaa gtgctgctct tgtgccagct tatcctgaga gaatatacga agctatgaga | 120 |
| tactccctcc tggcaggtgg caaaagatta agacctatct tatgtttagc tgcttgcgaa | 180 |
| ttggcaggtg gttctgttga acaagccatg ccaactgcgt gtgcacttga aatgatccat | 240 |
| acaatgtcac taattcatga tgacctgcca gccatggata acgatgattt cagaagagga | 300 |
| aagccaacta atcacaaggt gttcggggaa gatatagcca tcttagcggg tgatgcgctt | 360 |
| ttagcttacg cttttgaaca tattgcttct caaacaagag gagtaccacc tcaattggtg | 420 |
| ctacaagtta ttgctagaat cggacacgcc gttgctgcaa caggcctcgt tggaggccaa | 480 |
| gtcgtagacc ttgaatctga aggtaaagct atttccttag aaacattgga gtatattcac | 540 |
| tcacataaga ctggagcctt gctggaagca tcagttgtct caggcggtat tctcgcaggg | 600 |
| gcagatgaag agcttttggc cagattgtct cattacgcta gagatatagg cttggctttt | 660 |
| caaatcgtcg atgatatcct ggatgttact gctacatctg aacagttggg gaaaaccgct | 720 |
| ggtaaagacc aggcagccgc aaaggcaact tatccaagtc tattgggttt agaagcctct | 780 |
| agacagaaag cggaagagtt gattcaatct gctaaggaag ccttaagacc ttacggttca | 840 |
| caagcagagc cactcctagc gctggcagac ttcatcacac gtcgtcagca ttaa | 894 |

SEQ ID NO: 32
Synechococcus sp.

| | |
|---|---|
| MVAQTFNLDT YLSQRQQQVE EALSAALVPA YPERIYEAMR YSLLAGGKRL RPILCLAACE | 60 |
| LAGGSVEQAM PTACALEMIH TMSLIHDDLP AMDNDDFRRG KPTNHKVFGE DIAILAGDAL | 120 |
| LAYAFEHIAS QTRGVPPQLV LQVIARIGHA VAATGLVGGQ VVDLESEGKA ISLETLEYIH | 180 |
| SHKTGALLEA SVVSGGILAG ADEELLARLS HYARDIGLAF QIVDDILDVT ATSEQLGKTA | 240 |
| GKDQAAAKAT YPSLLGLEAS RQKAEELIQS AKEALRPYGS QAEPLLALAD FITRRQH | 297 |

SEQ ID NO: 33
Artificial Sequence

| | |
|---|---|
| atgaaaaccg ggtttatctc accagcaaca gtatttcatc acagaatctc accagcgacc | 60 |
| actttcagac atcacttatc acctgctact acaaactcta caggcattgt cgccttaaga | 120 |
| gacatcaact tcagatgtaa agcagtttct aaagagtact ctgatctgtt gcagaaagat | 180 |
| gaggcttctt tcacaaaatg ggacgatgac aaggtgaaag atcatcttga taccaacaaa | 240 |
| aacttatacc caaatgatga gattaaggaa tttgttgaat cagtaaaggc tatgttcggt | 300 |
| agtatgaatg acggggagat aaacgtctct gcatacgata ctgcatgggt tgctttggtt | 360 |
| caagatgtcg atggatcagg tagtcctcag ttcccttctt ctttagaatg gattgccaac | 420 |
| aatcaattgt cagatggatc atggggagat catttgctgt tctcagctca cgatagaatc | 480 |
| atcaacacat tagcatgcgt tattgcactt acaagttgga atgttcatcc ttctaagtgt | 540 |
| gaaaaaggtt tgaattttct gagagaaaac atttgcaaat tagaagatga aaacgcagaa | 600 |
| catatgccaa ttggttttga agtaacattc ccatcactaa ttgatatcgc gaaaaagttg | 660 |
| aacattgaag tacctgagga tactccagca cttaaagaga tctacgcacg tagagatatc | 720 |
| aagttaacta agatcccaat ggaagttctt cacaaggtac ctactacttt gttacattct | 780 |
| ttggaaggaa tgcctgattt ggagtgggaa aaactgttaa agctacaatg taaagatggt | 840 |
| agtttcttgt tttccccatc tagtaccgca ttcgccctaa tgcaaacaaa agatgagaaa | 900 |
| tgcttacagt atctaacaaa tatcgtcact aagttcaacg gtggcgtgcc taatgtgtac | 960 |
| ccagtcgatt tgtttgaaca tatttgggtt gttgatagac tgcagagatt ggggattgcc | 1020 |
| agatacttca aatcagagat aaaagattgt gtagagtata tcaataagta ctggaccaaa | 1080 |
| aatggaattt gttgggctag aaatactcac gttcaagata tcgatgatac agccatggga | 1140 |
| ttcagagtgt tgagagcgca cggttatgac gtcactccag atgtttttag acaatttgaa | 1200 |
| aaagatggta aattcgtttg ctttgcaggg caatcaacac aagccgtgac aggaatgttt | 1260 |
| aacgtttaca gagcctctca aatgttgttc ccaggggaga gaattttgga agatgccaaa | 1320 |
| aagttctctt acaattactt aaaggaaaag caaagtacca acgaattgct ggataaatgg | 1380 |
| ataatcgcta aagatctacc tggtgaagtt ggttatgctc tggatatccc atggtatgct | 1440 |
| tccttaccaa gattggaaac tcgttattac cttgaacaat acggcggtga agatgatgtc | 1500 |
| tggataggca agacattata cagaatgggt tacgtgtcca ataacacata tctagaaatg | 1560 |
| gcaaagctgg attacaataa ctatgttgca gtccttcaat tagaatggta cacaatacaa | 1620 |
| caatggtacg tcgatattgg tatagagaag ttcgaatctg acaacatcaa gtcagtcctg | 1680 |

SEQ ID NO: 34
Stevia rebaudiana

| | |
|---|---|
| MKTGFISPAT VFHHRISPAT TFRHHLSPAT TNSTGIVALR DINFRCKAVS KEYSDLLQKD | 60 |
| EASFTKWDDD KVKDHLDTNK NLYPNDEIKE FVESVKAMFG SMNDGEINVS AYDTAWVALV | 120 |
| QDVDGSGSPQ FPSSLEWIAN NQLSDGSWGD HLLFSAHDRI INTLACVIAL TSWNVHPSKC | 180 |
| EKGLNFLREN ICKLEDENAE HMPIGFEVTF PSLIDIAKKL NIEVPEDTPA LKEIYARRDI | 240 |
| KLTKIPMEVL HKVPTTLLHS LEGMPDLEWE KLLKLQCKDG SFLFSPSSTA FALMQTKDEK | 300 |
| CLQYLTNIVT KFNGGVPNVY PVDLFEHIWV VDRLQRLGIA RYFKSEIKDC VEYINKYWTK | 360 |
| NGICWARNTH VQDIDDTAMG FRVLRAHGYD VTPDVFRQFE KDGKFVCFAG QSTQAVTGMF | 420 |
| NVYRASQMLF PGERILEDAK KFSYNYLKEK QSTNELLDKW IIAKDLPGEV GYALDIPWYA | 480 |
| SLPRLETRYY LEQYGGEDDV WIGKTLYRMG YVSNNTYLEM AKLDYNNYVA VLQLEWYTIQ | 540 |
| QWYVDIGIEK FESDNIKSVL VSYYLAAASI FEPERSKERI AWAKTTILVD KITSIFDSSQ | 600 |
| SSKEDITAFI DKFRNKSSSK KHSINGEPWH EVMVALKKTL HGFALDALMT HSQDIHPQLH | 660 |
| QAWEMWLTKL QDGVDVTAEL MVQMINMTAG RWVSKELLTH PQYQRLSTVT NSVCHDITKL | 720 |
| HNFKENSTTV DSKVQELVQL VFSDTPDDLD QDMKQTFLTV MKTFYYKAWC DPNTINDHIS | 780 |
| KVFEIVI | 787 |

SEQ ID NO: 35
Artificial Sequence

| | |
|---|---|
| atgcctgatg cacacgatgc tccacctcca caaataagac agagaacact agtagatgag | 60 |
| gctacccaac tgctaactga gtccgcagaa gatgcatggg gtgaagtcag tgtgtcagaa | 120 |
| tacgaaacag caaggctagt tgcccatgct acatggttag gtggacacgc cacaagagtg | 180 |
| gccttccttc tggagagaca acacgaagac gggtcatggg gtccaccagg tggatatagg | 240 |
| ttagtcccta cattatctgc tgttcacgca ttattgacat gtcttgcctc tcctgctcag | 300 |
| gatcatggcg ttccacatga tagactttta agagctgttg acgcaggctt gactgccttg | 360 |
| agaagattgg ggacatctga ctccccacct gatactatag cagttgagct ggttatccca | 420 |
| tctttgctag agggcattca acacttactg gaccctgctc atcctcatag tagaccagcc | 480 |
| ttctctcaac atagaggctc tcttgtttgt cctggtggac tagatgggag aactctagga | 540 |
| gctttgagat cacacgccgc agcaggtaca ccagtaccag gaaaagtctg gcacgcttcc | 600 |
| gagactttgg gcttgagtac cgaagctgct tctcacttgc aaccagccca aggtataatc | 660 |
| ggtggctctg ctgctgccac agcaacatgg ctaaccaggg ttgcaccatc tcaacagtca | 720 |
| gattctgcca gaagatacct tgaggaatta caacacagat actctggccc agttccttcc | 780 |
| attaccccta tcacatactt cgaaagagca tggttattga acaattttgc agcagccggt | 840 |
| gttccttgtg aggctccagc tgctttgttg gattccttag aagcagcact tacaccacaa | 900 |
| ggtgctcctg ctggagcagg attgcctcca gatgctgatg atacagccgc tgtgttgctt | 960 |
| gcattggcaa cacatgggag aggtagaaga ccagaagtac tgatggatta caggactgac | 1020 |
| gggtatttcc aatgctttat tggggaaagg actccatcaa tttcaacaaa cgctcacgta | 1080 |
| ttggaaacat tagggcatca tgtggcccaa catccacaag atagagccag atacggatca | 1140 |
| gccatggata ccgcatcagc ttggctgctg gcagctcaaa agcaagatgg ctcttggtta | 1200 |
| gataaatggc atgcctcacc atactacgct actgtttgtt gcacacaagc cctagccgct | 1260 |
| catgcaagtc ctgcaactgc accagctaga cagagagctg tcagatgggt tttagccaca | 1320 |
| caaagatccg atggcggttg gggtctatgg cattcaactg ttgaagagac tgcttatgcc | 1380 |
| ttacagatct tggccccacc ttctggtggt ggcaatatcc cagtccaaca agcacttact | 1440 |
| agaggcagag caagattgtg tggagccttg ccactgactc ctttatggca tgataaggat | 1500 |
| ttgtatactc cagtaagagt agtcagagct gccagagctg ctgctctgta cactaccaga | 1560 |
| gatctattgt taccaccatt gtaa | 1584 |

SEQ ID NO: 36
Streptomyces clavuligerus

| | |
|---|---|
| MPDAHDAPPP QIRQRTLVDE ATQLLTESAE DAWGEVSVSE YETARLVAHA TWLGGHATRV | 60 |
| AFLLERQHED GSWGPPGGYR LVPTLSAVHA LLTCLASPAQ DHGVPHDRLL RAVDAGLTAL | 120 |
| RRLGTSDSPP DTIAVELVIP SLLEGIQHLL DPAHPHSRPA FSQHRGSLVC PGGLDGRTLG | 180 |
| ALRSHAAAGT PVPGKVWHAS ETLGLSTEAA SHLQPAQGII GGSAAATATW LTRVAPSQQS | 240 |
| DSARRYLEEL QHRYSGPVPS ITPITYFERA WLLNNFAAAG VPCEAPAALL DSLEAALTPQ | 300 |
| GAPAGAGLPP DADDTAAVLL ALATHGRGRR PEVLMDYRTD GYFQCFIGER TPSISTNAHV | 360 |
| LETLGHHVAQ HPQDRARYGS AMDTASAWLL AAQKQDGSWL DKWHASPYYA TVCCTQALAA | 420 |
| HASPATAPAR QRAVRWVLAT QRSDGGWGLW HSTVEETAYA LQILAPPSGG GNIPVQQALT | 480 |
| RGRARLCGAL PLTPLWHDKD LYTPVRVVRA ARAAALYTTR DLLLPPL | 527 |

SEQ ID NO: 37
Artificial Sequence

| | |
|---|---|
| atgaacgccc tatccgaaca cattttgtct gaattgagaa gattattgtc tgaaatgagt | 60 |
| gatggcggat ctgttggtcc atctgtgtat gatacggccc aggccctaag attccacggt | 120 |
| aacgtaacag gtagacaaga tgcatatgct tggttgatcg cccagcaaca agcagatgga | 180 |
| ggttggggct ctgccgactt tccactcttt agacatgctc caacatgggc tgcacttctc | 240 |
| gcattacaaa gagctgatcc acttcctggc gcagcagacg cagttcagac cgcaacaaga | 300 |
| ttcttgcaaa gacaaccaga tccatacgct catgccgttc ctgaggatgc ccctattggt | 360 |
| gctgaactga tcttgcctca gttttgtgga gaggctgctt ggttgttggg aggtgtggcc | 420 |
| ttccctagac acccagccct attaccatta agacaggctt gtttagtcaa actgggtgca | 480 |
| gtcgccatgt tgccttcagg acacccattg ctccactcct gggaggcatg gggtacttct | 540 |
| ccaacaacag cctgtccaga cgatgatggt tctataggta tctcaccagc agctacagcc | 600 |
| gcctggagag cccaggctgt gaccagaggc tcaactcctc aagtgggcag agctgacgca | 660 |
| tacttacaaa tggcttcaag agcaacgaga tcaggcatag aaggagtctt ccctaatgtt | 720 |
| tggcctataa acgtattcga accatgctgg tcactgtaca ctctccatct tgccggtctg | 780 |
| ttcgcccatc cagcactggc tgaggctgta agagttatcg ttgctcaact tgaagcaaga | 840 |
| ttgggagtgc atggcctcgg accagcttta cattttgctg ccgacgctga tgatactgca | 900 |
| gttgccttat gcgttctgca tttggctggc agagatcctg cagttgacgc attgagacat | 960 |
| tttgaaattg gtgagctctt tgttacattc ccaggagaga gaaatgctag tgtctctacg | 1020 |
| aacattcacg ctcttcatgc tttgagattg ttaggtaaac cagctgccgg agcaagtgca | 1080 |
| tacgtcgaag caaatagaaa tccacatggt ttgtgggaca acgaaaaatg gcacgtttca | 1140 |
| tggctttatc caactgcaca cgccgttgca gctctagctc aaggcaagcc tcaatggaga | 1200 |
| gatgaaagag cactagccgc tctactacaa gctcaaagag atgatggtgg ttggggagct | 1260 |
| ggtagaggat ccactttcga ggaaaccgcc tacgctcttt tcgctttaca cgttatggac | 1320 |
| ggatctgagg aagccacagg cagaagaaga atcgctcaag tcgtcgcaag agccttagaa | 1380 |
| tggatgctag ctagacatgc cgcacatgga ttaccacaaa caccactctg gattggtaag | 1440 |
| gaattgtact gtcctactag agtcgtaaga gtagctgagc tagctggcct gtggttagca | 1500 |
| ttaagatggg gtagaagagt attagctgaa ggtgctggtg ctgcacctta a | 1551 |

SEQ ID NO: 38
Bradyrhizobium japonicum

| | |
|---|---|
| MNALSEHILS ELRRLLSEMS DGGSVGPSVY DTAQALRFHG NVTGRQDAYA WLIAQQQADG | 60 |
| GWGSADFPLF RHAPTWAALL ALQRADPLPG AADAVQTATR FLQRQPDPYA HAVPEDAPIG | 120 |
| AELILPQFCG EAAWLLGGVA FPRHPALLPL RQACLVKLGA VAMLPSGHPL LHSWEAWGTS | 180 |
| PTTACPDDDG SIGISPAATA AWRAQAVTRG STPQVGRADA YLQMASRATR SGIEGVFPNV | 240 |
| WPINVFEPCW SLYTLHLAGL FAHPALAEAV RVIVAQLEAR LGVHGLGPAL HFAADADDTA | 300 |
| VALCVLHLAG RDPAVDALRH FEIGELFVTF PGERNASVST NIHALHALRL LGKPAAGASA | 360 |
| YVEANRNPHG LWDNEKWHVS WLYPTAHAVA ALAQGKPQWR DERALAALLQ AQRDDGGWGA | 420 |
| GRGSTFEETA YALFALHVMD GSEEATGRRR IAQVVARALE WMLARHAAHG LPQTPLWIGK | 480 |
| ELYCPTRVVR VAELAGLWLA LRWGRRVLAE GAGAAP | 516 |

SEQ ID NO: 39
Artificial Sequence

| | |
|---|---|
| atggttttgt cttcttcttg tactacagta ccacacttat cttcattagc tgtcgtgcaa | 60 |
| cttggtcctt ggagcagtag gattaaaaag aaaaccgata ctgttgcagt accagccgct | 120 |
| gcaggaaggt ggagaagggc cttggctaga gcacagcaca catcagaatc cgcagctgtc | 180 |
| gcaaagggca gcagtttgac ccctatagtg agaactgacg ctgagtcaag gagaacaaga | 240 |
| tggccaaccg atgacgatga cgccgaacct ttagtggatg agatcagggc aatgcttact | 300 |
| tccatgtctg atggtgacat ttccgtgagc gcatacgata cagcctgggt cggattggtt | 360 |
| ccaagattag acggcggtga aggtcctcaa tttccagcag ctgtgagatg gataagaaat | 420 |
| aaccagttgc ctgacggaag ttggggcgat gccgcattat tctctgccta tgacaggctt | 480 |
| atcaataccc ttgcctgcgt tgtaactttg acaaggtggt ccctagaacc agagatgaga | 540 |
| ggtagaggac tatctttttt gggtaggaac atgtggaaat tagcaactga agatgaagag | 600 |
| tcaatgccta ttggcttcga attagcattt ccatctttga tagagcttgc taagagccta | 660 |
| ggtgtccatg acttccctta tgatcaccag gccctacaag gaatctactc ttcaagagag | 720 |
| atcaaaatga agaggattcc aaaagaagtg atgcataccg ttccaacatc aatattgcac | 780 |
| agtttggagg gtatgcctgg cctagattgg gctaaactac ttaaactaca gagcagcgac | 840 |
| ggaagttttt tgttctcacc agctgccact gcatatgctt taatgaatac cggagatgac | 900 |
| aggtgtttta gctacatcga tagaacagta aagaaattca acggcggcgt ccctaatgtt | 960 |
| tatccagtgg atctatttga acatatttgg gccgttgata gacttgaaag attaggaatc | 1020 |
| tccaggtact tccaaaagga gatcgaacaa tgcatggatt atgtaaacag gcattggact | 1080 |
| gaggacggta tttgttgggc aaggaactct gatgtcaaag aggtggacga cacagctatg | 1140 |
| gcctttagac ttcttaggtt gcacggctac agcgtcagtc ctgatgtgtt taaaaacttc | 1200 |
| gaaaaggacg gtgaattttt cgcatttgtc ggacagtcta atcaagctgt taccggtatg | 1260 |
| tacaacttaa acagagcaag ccagatatcc ttcccaggcg aggatgtgct tcatagagct | 1320 |
| ggtgccttct catatgagtt cttgaggaga aaagaagcag agggagcttt gagggacaag | 1380 |
| tggatcattt ctaaagatct acctggtgaa gttgtgtata ctttggattt tccatggtac | 1440 |
| ggcaacttac ctagagtcga ggccagagac tacctagagc aatacggagg tggtgatgac | 1500 |
| gtttggattg gcaagacatt gtataggatg ccacttgtaa acaatgatgt atatttggaa | 1560 |
| ttggcaagaa tggatttcaa ccactgccag gctttgcatc agttagagtg gcaaggacta | 1620 |
| aaaagatggt atactgaaaa taggttgatg gactttggtg tcgcccaaga agatgccctt | 1680 |
| agagcttatt ttcttgcagc cgcatctgtt tacgagcctt gtagagctgc cgagaggctt | 1740 |
| gcatgggcta gagccgcaat actagctaac gccgtgagca cccacttaag aaatagccca | 1800 |
| tcattcagag aaaggttaga gcattctctt aggtgtagac ctagtgaaga gacagatggc | 1860 |
| tcctggttta actcctcaag tggctctgat gcagttttag taaaggctgt cttaagactt | 1920 |
| actgattcat tagccaggga agcacagcca atccatggag gtgacccaga agatattata | 1980 |
| cacaagttgt taagatctgc ttgggccgag tgggttaggg aaaaggcaga cgctgccgat | 2040 |
| agcgtgtgca atggtagttc tgcagtagaa caagagggat caagaatggt ccatgataaa | 2100 |
| cagacctgtc tattattggc tagaatgatc gaaatttctg ccggtagggc agctggtgaa | 2160 |
| gcagccagtg aggacggcga tagaagaata attcaattaa caggctccat ctgcgacagt | 2220 |
| cttaagcaaa aaatgctagt ttcacaggac cctgaaaaaa atgaagagat gatgtctcac | 2280 |
| gtggatgacg aattgaagtt gaggattaga gagttcgttc aatatttgct tagactaggt | 2340 |
| gaaaaaaaga ctggatctag cgaaaccagg caaacatttt taagtatagt gaaatcatgt | 2400 |
| tactatgctg ctcattgccc acctcatgtc gttgatagac acattagtag agtgattttc | 2460 |
| gagccagtaa gtgccgcaaa gtaaccgcgg | 2490 |

SEQ ID NO: 40
Zea mays

| | |
|---|---|
| MVLSSSCTTV PHLSSLAVVQ LGPWSSRIKK KTDTVAVPAA AGRWRRALAR AQHTSESAAV | 60 |
| AKGSSLTPIV RTDAESRRTR WPTDDDDAEP LVDEIRAMLT SMSDGDISVS AYDTAWVGLV | 120 |
| PRLDGGEGPQ FPAAVRWIRN NQLPDGSWGD AALFSAYDRL INTLACVVTL TRWSLEPEMR | 180 |
| GRGLSFLGRN MWKLATEDEE SMPIGFELAF PSLIELAKSL GVHDFPYDHQ ALQGIYSSRE | 240 |
| IKMKRIPKEV MHTVPTSILH SLEGMPGLDW AKLLKLQSSD GSFLFSPAAT AYALMNTGDD | 300 |
| RCFSYIDRTV KKFNGGVPNV YPVDLFEHIW AVDRLERLGI SRYFQKEIEQ CMDYVNRHWT | 360 |
| EDGICWARNS DVKEVDDTAM AFRLLRLHGY SVSPDVFKNF EKDGEFFAFV GQSNQAVTGM | 420 |
| YNLNRASQIS FPGEDVLHRA GAFSYEFLRR KEAEGALRDK WIISKDLPGE VVYTLDFPWY | 480 |
| GNLPRVEARD YLEQYGGGDD VWIGKTLYRM PLVNNDVYLE LARMDFNHCQ ALHQLEWQGL | 540 |
| KRWYTENRLM DFGVAQEDAL RAYFLAAASV YEPCRAAERL AWARAAILAN AVSTHLRNSP | 600 |
| SFRERLEHSL RCRPSEETDG SWFNSSSGSD AVLVKAVLRL TDSLAREAQP IHGGDPEDII | 660 |
| HKLLRSAWAE WVREKADAAD SVCNGSSAVE QEGSRMVHDK QTCLLLARMI EISAGRAAGE | 720 |
| AASEDGDRRI IQLTGSICDS LKQKMLVSQD PEKNEEMMSH VDDELKLRIR EFVQYLLRLG | 780 |
| EKKTGSSETR QTFLSIVKSC YYAAHCPPHV VDRHISRVIF EPVSAAK | 827 |

SEQ ID NO: 41
Artificial Sequence

| | |
|---|---|
| cttcttcact aaatacttag acagagaaaa cagagctttt taaagccatg tctcttcagt | 60 |
| atcatgttct aaactccatt ccaagtacaa cctttctcag ttctactaaa acaacaatat | 120 |
| cttcttcttt ccttaccatc tcaggatctc ctctcaatgt cgctagagac aaatccagaa | 180 |
| gcggttccat acattgttca aagcttcgaa ctcaagaata cattaattct caagaggttc | 240 |
| aacatgattt gcctctaata catgagtggc aacagcttca aggagaagat gctcctcaga | 300 |
| ttagtgttgg aagtaatagt aatgcattca aagaagcagt gaagagtgtg aaaacgatct | 360 |
| tgagaaacct aacggacggg gaaattacga tatcggctta cgatacagct tgggttgcat | 420 |
| tgatcgatgc cggagataaa actccggcgt ttccctccgc cgtgaaatgg atcgccgaga | 480 |
| accaactttc cgatggttct tggggagatg cgtatctctt ctcttatcat gatcgtctca | 540 |
| tcaataccct tgcatgcgtc gttgctctaa gatcatggaa tctctttcct catcaatgca | 600 |
| acaaaggaat cacgtttttc cgggaaaata ttgggaagct agaagacgaa aatgatgagc | 660 |
| atatgccaat cggattcgaa gtagcattcc catcgttgct tgagatagct cgaggaataa | 720 |
| acattgatgt accgtacgat tctccggtct taaaagatat atacgccaag aaagagctaa | 780 |
| agcttacaag gataccaaaa gagataatgc acaagatacc aacaacattg ttgcatagtt | 840 |
| tggaggggat gcgtgattta gattgggaaa agctcttgaa acttcaatct caagacggat | 900 |
| ctttcctctt ctctccttcc tctaccgctt ttgcattcat gcagacccga gacagtaact | 960 |
| gcctcgagta tttgcgaaat gccgtcaaac gtttcaatgg aggagttccc aatgtctttc | 1020 |
| ccgtggatct tttcgagcac atatggatag tggatcggtt acaacgttta gggatatcga | 1080 |
| gatactttga agaagagatt aaagagtgtc ttgactatgt ccacagatat tggaccgaca | 1140 |
| atggcatatg ttgggctaga tgttcccatg tccaagacat cgatgataca gccatggcat | 1200 |
| ttaggctctt aagacaacat ggataccaag tgtccgcaga tgtattcaag aactttgaga | 1260 |
| aagagggaga gtttttctgc tttgtggggc aatcaaacca agcagtaacc ggtatgttca | 1320 |
| acctataccg ggcatcacaa ttggcgtttc caagggaaga gatattgaaa aacgccaaag | 1380 |
| agttttctta taattatctg ctagaaaaac gggagagaga ggagttgatt gataagtgga | 1440 |
| ttataatgaa agacttacct ggcgagattg ggtttgcgtt agagattcca tggtacgcaa | 1500 |
| gcttgcctcg agtagagacg agattctata ttgatcaata tggtggagaa aacgacgttt | 1560 |
| ggattggcaa gactctttat aggatgccat acgtgaacaa taatggatat ctggaattag | 1620 |
| caaaacaaga ttacaacaat tgccaagctc agcatcagct cgaatgggac atattccaaa | 1680 |
| agtggtatga agaaaatagg ttaagtgagt ggggtgtgcg cagaagtgag cttctcgagt | 1740 |
| gttactactt agcggctgca actatatttg aatcagaaag gtcacatgag agaatggttt | 1800 |
| gggctaagtc aagtgtattg gttaaagcca tttcttcttc ttttggggaa tcctctgact | 1860 |
| ccagaagaag cttctccgat cagtttcatg aatacattgc caatgctcga cgaagtgatc | 1920 |
| atcactttaa tgacaggaac atgagattgg accgaccagg atcggttcag gccagtcggc | 1980 |
| ttgccggagt gttaatcggg actttgaatc aaatgtcttt tgaccttttc atgtctcatg | 2040 |
| gccgtgacgt taacaatctc ctctatctat cgtggggaga ttggatggaa aaatggaaac | 2100 |
| tatatggaga tgaaggagaa ggagagctca tggtgaagat gataattcta atgaagaaca | 2160 |
| atgacctaac taacttcttc acccacactc acttcgttcg tctcgcggaa atcatcaatc | 2220 |
| gaatctgtct tcctcgccaa tacttaaagg caaggagaaa cgatgagaag gagaagacaa | 2280 |
| taaagagtat ggagaaggag atggggaaaa tggttgagtt agcattgtcg gagagtgaca | 2340 |
| catttcgtga cgtcagcatc acgtttcttg atgtagcaaa agcattttac tactttgctt | 2400 |
| tatgtggcga tcatctccaa actcacatct ccaaagtctt gtttcaaaaa gtctagtaac | 2460 |
| ctcatcatca tcatcgatcc attaacaatc agtggatcga tgtatccata gatgcgtgaa | 2520 |
| taatatttca tgtagagaag gagaacaaat tagatcatgt agggttatca | 2570 |

SEQ ID NO: 42
Arabidopsis thaliana

| | |
|---|---|
| MSLQYHVLNS IPSTTFLSST KTTISSSFLT ISGSPLNVAR DKSRSGSIHC SKLRTQEYIN | 60 |
| SQEVQHDLPL IHEWQQLQGE DAPQISVGSN SNAFKEAVKS VKTILRNLTD GEITISAYDT | 120 |
| AWVALIDAGD KTPAFPSAVK WIAENQLSDG SWGDAYLFSY HDRLINTLAC VVALRSWNLF | 180 |
| PHQCNKGITF FRENIGKLED ENDEHMPIGF EVAFPSLLEI ARGINIDVPY DSPVLKDIYA | 240 |
| KKELKLTRIP KEIMHKIPTT LLHSLEGMRD LDWEKLLKLQ SQDGSFLFSP SSTAFAFMQT | 300 |
| RDSNCLEYLR NAVKRFNGGV PNVFPVDLFE HIWIVDRLQR LGISRYFEEE IKECLDYVHR | 360 |
| YWTDNGICWA RCSHVQDIDD TAMAFRLLRQ HGYQVSADVF KNFEKEGEFF CFVGQSNQAV | 420 |
| TGMFNLYRAS QLAFPREEIL KNAKEFSYNY LLEKREREEL IDKWIIMKDL PGEIGFALEI | 480 |
| PWYASLPRVE TRFYIDQYGG ENDVWIGKTL YRMPYVNNNG YLELAKQDYN NCQAQHQLEW | 540 |
| DIFQKWYEEN RLSEWGVRRS ELLECYYLAA ATIFESERSH ERMVWAKSSV LVKAISSSFG | 600 |
| ESSDSRRSFS DQFHEYIANA RRSDHHFNDR NMRLDRPGSV QASRLAGVLI GTLNQMSFDL | 660 |
| FMSHGRDVNN LLYLSWGDWM EKWKLYGDEG EGELMVKMII LMKNNDLTNF FTHTHFVRLA | 720 |
| EIINRICLPR QYLKARRNDE KEKTIKSMEK EMGKMVELAL SESDTFRDVS ITFLDVAKAF | 780 |
| YYFALCGDHL QTHISKVLFQ KV | 802 |

SEQ ID NO: 43
Artificial Sequence

| | |
|---|---|
| atgaatttga gtttgtgtat agcatctcca ctattgacca aatctaatag accagctgct | 60 |
| ttatcagcaa ttcatacagc tagtacatcc catggtggcc aaaccaaccc tacgaatctg | 120 |
| ataatcgata cgaccaagga gagaatacaa aaacaattca aaaatgttga aatttcagtt | 180 |
| tcttcttatg atactgcgtg ggttgccatg gttccatcac ctaattctcc aaagtctcca | 240 |
| tgtttcccag aatgtttgaa ttggctgatt aacaaccagt tgaatgatgg atcttggggt | 300 |
| ttagtcaatc acacgcacaa tcacaaccat ccacttttga aagattcttt atcctcaact | 360 |
| ttggcttgca tcgtggccct aaagagatgg aacgtaggtg aggatcagat taacaagggg | 420 |
| cttagtttca ttgaatctaa cttggcttcc gcgactgaaa aatctcaacc atctccaata | 480 |
| ggattcgata tcatctttcc aggtctgtta gagtacgcca aaaatctaga tatcaactta | 540 |
| ctgtctaagc aaactgattt ctcactaatg ttacacaaga gagaattaga acaaaagaga | 600 |
| tgtcattcaa acgaaatgga tggttaccta gcttatatct ctgaaggtct tggtaatctt | 660 |
| tacgattgga atatggtgaa aaagtaccag atgaaaaatg gctcagtttt caattcccct | 720 |
| tctgcaactg cggcagcatt cattaaccat caaaatccag gatgcctgaa ctatttgaat | 780 |
| tcactactag acaaattcgg caacgcagtt ccaactgtat accctcacga tttgtttatc | 840 |
| agattgagta tggtggatac aattgaaaga cttggtatat cccaccactt tagagtcgag | 900 |
| atcaaaaatg ttttggatga gacataccgt tgttgggtgg agagagatga acaaatcttt | 960 |
| atggatgttg tgacgtgcgc gttggccttt agattgttgc gtattaacgg ttacgaagtt | 1020 |
| agtccagatc cacttgccga aattacaaac gaattagctt taaaggatga atacgccgct | 1080 |
| cttgaaacat atcatgcgtc acatatcctt taccaagagg acttatcatc tggaaaacaa | 1140 |
| attcttaaat ctgctgattt cctgaaggaa atcatatcca ctgatagtaa tagactgtcc | 1200 |
| aaactgatcc ataaagaggt tgaaaatgca cttaagttcc ctattaacac cggcttagaa | 1260 |
| cgtattaaca caagacgtaa catccagctt tacaacgtag acaatactag aatcttgaaa | 1320 |
| accacttacc attcttccaa catatcaaac actgattacc taagattagc tgttgaagat | 1380 |
| ttctacacat gtcagtctat ctatagagaa gagctgaaag gattagagag atgggtcgtt | 1440 |
| gagaataagc tagatcaatt gaaatttgcc agacaaaaga cagcttattg ttacttctca | 1500 |
| gttgccgcca ctttatcaag tccagaattg tcagatgcac gtatttcttg ggctaaaaac | 1560 |
| ggaattttga caactgttgt tgatgatttc tttgatattg gcgggacaat cgacgaattg | 1620 |
| acaaacctga ttcaatgcgt tgaaaagtgg aatgtcgatg tcgataaaga ctgttgctca | 1680 |
| gaacatgtta gaatactgtt cttggctctg aaagatgcta tctgttggat cggggatgag | 1740 |
| gctttcaaat ggcaagctag agatgtgacg tctcacgtca ttcaaacctg gctagaactg | 1800 |
| atgaactcta tgttgagaga agcaatttgg actagagatg catacgttcc tacattaaac | 1860 |
| gagtatatgg aaaacgctta tgtctccttt gctttgggtc ctatcgttaa gcctgccata | 1920 |
| tactttgtag gaccaaagct atccgaggaa atcgtcgaat catcagaata ccataacttg | 1980 |
| ttcaagttaa tgtccacaca aggcagatta cttaatgata ttcattcttt caaaagagag | 2040 |
| tttaaggaag gaaagttaaa tgctgttgct ctgcatcttt ctaatggcga aagtggtaaa | 2100 |
| gtcgaagagg aagtagttga ggaaatgatg atgatgatca aaaacaagag aaaggagttg | 2160 |
| atgaaactaa tcttcgaaga gaacggttca attgttccta gagcatgtaa ggatgcattt | 2220 |
| tggaacatgt gtcatgtgct aaactttttc tacgcaaacg acgatggttt tactgggaac | 2280 |
| acaatactag atacagtaaa agacatcata tacaaccctt tggtcttagt aaacgaaaac | 2340 |
| gaggagcaaa gataa | 2355 |

SEQ ID NO: 44
Stevia rebaudiana

| | |
|---|---|
| MNLSLCIASP LLTKSNRPAA LSAIHTASTS HGGQTNPTNL IIDTTKERIQ KQFKNVEISV | 60 |
| SSYDTAWVAM VPSPNSPKSP CFPECLNWLI NNQLNDGSWG LVNHTHNHNH PLLKDSLSST | 120 |
| LACIVALKRW NVGEDQINKG LSFIESNLAS ATEKSQPSPI GFDIIFPGLL EYAKNLDINL | 180 |
| LSKQTDFSLM LHKRELEQKR CHSNEMDGYL AYISEGLGNL YDWNMVKKYQ MKNGSVFNSP | 240 |
| SATAAAFINH QNPGCLNYLN SLLDKFGNAV PTVYPHDLFI RLSMVDTIER LGISHHFRVE | 300 |
| IKNVLDETYR CWVERDEQIF MDVVTCALAF RLLRINGYEV SPDPLAEITN ELALKDEYAA | 360 |
| LETYHASHIL YQEDLSSGKQ ILKSADFLKE IISTDSNRLS KLIHKEVENA LKFPINTGLE | 420 |
| RINTRRNIQL YNVDNTRILK TTYHSSNISN TDYLRLAVED FYTCQSIYRE ELKGLERWVV | 480 |
| ENKLDQLKFA RQKTAYCYFS VAATLSSPEL SDARISWAKN GILTTVVDDF FDIGGTIDEL | 540 |
| TNLIQCVEKW NVDVDKDCCS EHVRILFLAL KDAICWIGDE AFKWQARDVT SHVIQTWLEL | 600 |
| MNSMLREAIW TRDAYVPTLN EYMENAYVSF ALGPIVKPAI YFVGPKLSEE IVESSEYHNL | 660 |
| FKLMSTQGRL LNDIHSFKRE FKEGKLNAVA LHLSNGESGK VEEEVVEEMM MMIKNKRKEL | 720 |
| MKLIFEENGS IVPRACKDAF WNMCHVLNFF YANDDGFTGN TILDTVKDII YNPLVLVNEN | 780 |
| EEQR | 784 |

SEQ ID NO: 45
Artificial Sequence

| | |
|---|---|
| atgaatctgt ccctttgtat agctagtcca ctgttgacaa aatcttctag accaactgct | 60 |
| ctttctgcaa ttcatactgc cagtactagt catggaggtc aaacaaaccc aacaaatttg | 120 |
| ataatcgata ctactaagga gagaatccaa aagctattca aaaatgttga aatctcagta | 180 |
| tcatcttatg acaccgcatg ggttgcaatg gtgccatcac ctaattcccc aaaaagtcca | 240 |
| tgttttccag agtgcttgaa ttggttaatc aataatcagt taaacgatgg ttcttggggt | 300 |
| ttagtcaacc acactcataa ccacaatcat ccattattga aggactcttt atcatcaaca | 360 |
| ttagcctgta ttgttgcatt gaaaagatgg aatgtaggtg aagatcaaat caacaagggt | 420 |
| ttatcattca tagaatccaa tctagcttct gctaccgaca aatcacaacc atctccaatc | 480 |
| gggttcgaca taatcttccc tggtttgctg gagtatgcca aaaaccttga tatcaactta | 540 |
| ctgtctaaac aaacagattt ctctttgatg ctacacaaaa gagagttaga gcagaaaaga | 600 |
| tgccattcta acgaaattga cgggtactta gcatatatct cagaaggttt gggtaatttg | 660 |
| tatgactgga acatggtcaa aaagtatcag atgaaaaatg gatccgtatt caattctcct | 720 |
| tctgcaactg ccgcagcatt cattaatcat caaaaccctg ggtgtcttaa ctacttgaac | 780 |
| tcactattag ataagtttgg aaatgcagtt ccaacagtct atcctttgga cttgtacatc | 840 |
| agattatcta tggttgacac tatagagaga ttaggtattt ctcatcattt cagagttgag | 900 |
| atcaaaaatg ttttggacga gacatacaga tgttgggtcg aaagagatga gcaaatcttt | 960 |
| atggatgtcg tgacctgcgc tctggctttt agattgctaa ggatacacgg atacaaagta | 1020 |
| tctcctgatc aactggctga gattacaaac gaactggctt tcaaagacga atacgccgca | 1080 |
| ttagaaacat accatgcatc ccaaatactt taccaggaag acctaagttc aggaaaacaa | 1140 |
| atcttgaagt ctgcagattt cctgaaaggc attctgtcta cagatagtaa taggttgtct | 1200 |
| aaattgatac acaaggaagt agaaaacgca ctaaagtttc ctattaacac tggtttagag | 1260 |
| agaatcaata ctaggagaaa cattcagctg tacaacgtag ataatacaag gattcttaag | 1320 |
| accacctacc atagttcaaa catttccaac acctattact taagattagc tgtcgaagac | 1380 |
| ttttacactt gtcaatcaat ctacagagag gagttaaagg gcctagaaag atgggtagtt | 1440 |
| caaaacaagt tggatcaact gaagtttgct agacagaaga cagcatactg ttatttctct | 1500 |
| gttgctgcta ccctttcatc cccagaattg tctgatgcca gaataagttg ggccaaaaat | 1560 |
| ggtattctta caactgtagt cgatgatttc tttgatattg gaggtactat tgatgaactg | 1620 |
| acaaatctta ttcaatgtgt tgaaaagtgg aacgtggatg tagataagga ttgctgcagt | 1680 |
| gaacatgtga gaatactttt cctggctcta aaagatgcaa tatgttggat tggcgacgag | 1740 |
| gccttcaagt ggcaagctag agatgttaca tctcatgtca tccaaacttg gcttgaactg | 1800 |
| atgaactcaa tgctaagaga agcaatctgg acaagagatg catacgttcc aacattgaac | 1860 |
| gaatacatgg aaaacgctta cgtctcattt gccttgggtc ctattgttaa gccagccata | 1920 |
| tactttgttg ggccaaagtt atccgaagag attgttgagt cttccgaata tcataaccta | 1980 |
| ttcaagttaa tgtcaacaca aggcagactt ctgaacgata tccactcctt caaaagagaa | 2040 |
| ttcaaggaag gtaagctaaa cgctgttgct ttgcacttgt ctaatggtga atctggcaaa | 2100 |
| gtggaagagg aagtcgttga ggaaatgatg atgatgatca aaaacaagag aaaggaattg | 2160 |
| atgaaattga ttttcgagga aaatggttca atcgtaccta gagcttgtaa agatgctttt | 2220 |
| tggaatatgt gccatgttct taacttcttt tacgctaatg atgatggctt cactggaaat | 2280 |
| acaatattgg atacagttaa agatatcatc tacaacccac ttgttttggt caatgagaac | 2340 |
| gaggaacaaa gataa | 2355 |

SEQ ID NO: 46
Stevia rebaudiana

| | |
|---|---|
| MNLSLCIASP LLTKSSRPTA LSAIHTASTS HGGQTNPTNL IIDTTKERIQ KLFKNVEISV | 60 |
| SSYDTAWVAM VPSPNSPKSP CFPECLNWLI NNQLNDGSWG LVNHTHNHNH PLLKDSLSST | 120 |
| LACIVALKRW NVGEDQINKG LSFIESNLAS ATDKSQPSPI GFDIIFPGLL EYAKNLDINL | 180 |
| LSKQTDFSLM LHKRELEQKR CHSNEIDGYL AYISEGLGNL YDWNMVKKYQ MKNGSVFNSP | 240 |
| SATAAAFINH QNPGCLNYLN SLLDKFGNAV PTVYPLDLYI RLSMVDTIER LGISHHFRVE | 300 |
| IKNVLDETYR CWVERDEQIF MDVVTCALAF RLLRIHGYKV SPDQLAEITN ELAFKDEYAA | 360 |
| LETYHASQIL YQEDLSSGKQ ILKSADFLKG ILSTDSNRLS KLIHKEVENA LKFPINTGLE | 420 |
| RINTRRNIQL YNVDNTRILK TTYHSSNISN TYYLRLAVED FYTCQSIYRE ELKGLERWVV | 480 |
| QNKLDQLKFA RQKTAYCYFS VAATLSSPEL SDARISWAKN GILTTVVDDF FDIGGTIDEL | 540 |
| TNLIQCVEKW NVDVDKDCCS EHVRILFLAL KDAICWIGDE AFKWQARDVT SHVIQTWLEL | 600 |
| MNSMLREAIW TRDAYVPTLN EYMENAYVSF ALGPIVKPAI YFVGPKLSEE IVESSEYHNL | 660 |
| FKLMSTQGRL LNDIHSFKRE FKEGKLNAVA LHLSNGESGK VEEEVVEEMM MMIKNKRKEL | 720 |
| MKLIFEENGS IVPRACKDAF WNMCHVLNFF YANDDGFTGN TILDTVKDII YNPLVLVNEN | 780 |
| EEQR | 784 |

SEQ ID NO: 47
Artificial Sequence

| | |
|---|---|
| atggctatgc cagtgaagct aacacctgcg tcattatcct taaaagctgt gtgctgcaga | 60 |
| ttctcatccg gtggccatgc tttgagattc gggagtagtc tgccatgttg gagaaggacc | 120 |
| cctacccaaa gatctacttc ttcctctact actagaccag ctgccgaagt gtcatcaggt | 180 |
| aagagtaaac aacatgatca ggaagctagt gaagcgacta tcagacaaca attacaactt | 240 |
| gtggatgtcc tggagaatat gggaatatcc agacattttg ctgcagagat aaagtgcata | 300 |
| ctagacagaa cttacagatc ttggttacaa agacacgagg aaatcatgct ggacactatg | 360 |
| acatgtgcta tggcttttag aatcctaaga ttgaacggat acaacgtttc atcagatgaa | 420 |
| ctataccacg ttgtagaggc atctggtctg cataattctt tgggtgggta tcttaacgat | 480 |
| accagaacac tacttgaatt acacaaggct tcaacagtta gtatctctga ggatgaatct | 540 |
| atcttagatt caattggctc tagatccaga acattgctta gagaacaatt ggagtctggt | 600 |
| ggcgcactga gaaagccttc tttattcaaa gaggttgaac atgcactgga tggacctttt | 660 |
| tacaccacac ttgatagact tcatcatagg tggaatattg aaaacttcaa cattattgag | 720 |
| caacacatgt tggagactcc atacttatct aaccagcata catcaaggga tatcctagca | 780 |
| ttgtcaatta gagatttttc ctcctcacaa ttcacttatc aacaagagct acagcatctg | 840 |
| gagagttggg ttaaggaatg tagattagat caactacagt tcgcaagaca gaaattagcg | 900 |
| tacttttacc tatcagccgc aggcaccatg ttttctcctg agctttctga tgcgagaaca | 960 |
| ttatgggcca aaaacggggt gttgacaact attgttgatg atttctttga tgttgccggt | 1020 |
| tctaaagagg aattggaaaa cttagtcatg ctggtcgaaa tgtgggatga acatcacaaa | 1080 |
| gttgaattct attctgagca ggtcgaaatc atcttctctt ccatctacga ttctgtcaac | 1140 |
| caattgggtg agaaggcctc tttggttcaa gacagatcaa ttacaaaaca ccttgttgaa | 1200 |
| atatggttag acttgttaaa gtccatgatg acggaagttg aatggagact gtcaaaatac | 1260 |
| gtgcctacag aaaaggaata catgattaat gcctctctta tcttcggcct aggtccaatc | 1320 |
| gttttaccag ctttgtattt cgttggtcca aagatttcag aaagtatagt aaaggaccca | 1380 |
| gaatatgatg aattgttcaa actaatgtca acatgtggta gattgttgaa tgacgtgcaa | 1440 |
| acgttcgaaa gagaatacaa tgagggtaaa ctgaattctg tcagtctatt ggttcttcac | 1500 |
| ggaggcccaa tgtctatttc agacgcaaag aggaaattac aaaagcctat tgatacgtgt | 1560 |
| agaagagatc ttctttcttt ggtccttaga gaagagtctg tagtaccaag accatgtaag | 1620 |
| gaactattct ggaaaatgtg taaagtgtgc tatttctttt actcaacaac tgatgggttt | 1680 |
| tctagtcaag tcgaaagagc aaaagaggta gacgctgtca taaatgagcc actgaagttg | 1740 |
| caaggttctc atacactggt atctgatgtt taa | 1773 |

SEQ ID NO: 48
Zea mays

| | |
|---|---|
| MAMPVKLTPA SLSLKAVCCR FSSGGHALRF GSSLPCWRRT PTQRSTSSST TRPAAEVSSG | 60 |
| KSKQHDQEAS EATIRQQLQL VDVLENMGIS RHFAAEIKCI LDRTYRSWLQ RHEEIMLDTM | 120 |
| TCAMAFRILR LNGYNVSSDE LYHVVEASGL HNSLGGYLND TRTLLELHKA STVSISEDES | 180 |
| ILDSIGSRSR TLLREQLESG GALRKPSLFK EVEHALDGPF YTTLDRLHHR WNIENFNIIE | 240 |
| QHMLETPYLS NQHTSRDILA LSIRDFSSSQ FTYQQELQHL ESWVKECRLD QLQFARQKLA | 300 |
| YFYLSAAGTM FSPELSDART LWAKNGVLTT IVDDFFDVAG SKEELENLVM LVEMWDEHHK | 360 |
| VEFYSEQVEI IFSSIYDSVN QLGEKASLVQ DRSITKHLVE IWLDLLKSMM TEVEWRLSKY | 420 |
| VPTEKEYMIN ASLIFGLGPI VLPALYFVGP KISESIVKDP EYDELFKLMS TCGRLLNDVQ | 480 |
| TFEREYNEGK LNSVSLLVLH GGPMSISDAK RKLQKPIDTC RRDLLSLVLR EESVVPRPCK | 540 |
| ELFWKMCKVC YFFYSTTDGF SSQVERAKEV DAVINEPLKL QGSHTLVSDV | 590 |

SEQ ID NO: 49
Artificial Sequence

| | |
|---|---|
| atgcagaact tccatggtac aaaggaaagg atcaaaaaga tgtttgacaa gattgaattg | 60 |
| tccgtttctt cttatgatac agcctgggtt gcaatggtcc catcccctga ttgcccagaa | 120 |
| acaccttgtt ttccagaatg tactaaatgg atcctagaaa atcagttggg tgatggtagt | 180 |
| tggtcacttc ctcatggcaa tccacttcta gttaaagatg cattatcttc cactcttgct | 240 |
| tgtattctgg ctcttaaaag atggggaatc ggtgaggaac agattaacaa aggactgaga | 300 |
| ttcatagaac tcaactctgc tagtgtaacc gataacgaac aacacaaacc aattggattt | 360 |
| gacattatct ttccaggtat gattgaatac gctatagact tagacctgaa tctaccacta | 420 |
| aaaccaactg acattaactc catgttgcat cgtagagccc ttgaattgac atcaggtgga | 480 |
| ggcaaaaatc tagaaggtag aagagcttac ttggcctacg tctctgaagg aatcggtaag | 540 |
| ctgcaagatt gggaaatggc tatgaaatac caacgtaaaa acggatctct gttcaatagt | 600 |
| ccatcaacaa ctgcagctgc attcatccat atacaagatg ctgaatgcct ccactatatt | 660 |
| cgttctcttc tccagaaatt tggaaacgca gtccctacaa tataccctct cgatatctat | 720 |
| gccagacttt caatggtaga tgccctggaa cgtcttggta ttgatagaca tttcagaaag | 780 |
| gagagaaagt tcgttctgga tgaaacatac agattttggt tgcaaggaga agaggagatt | 840 |
| ttctccgata acgcaacctg tgctttggcc ttcagaatat tgagacttaa tggttacgat | 900 |
| gtctctcttg aagatcactt ctctaactct ctgggcggtt acttaaagga ctcaggagca | 960 |
| gctttagaac tgtacagagc cctccaattg tcttacccag acgagtccct cctggaaaag | 1020 |
| caaaattcta gaacttctta cttcttaaaa caaggtttat ccaatgtctc cctctgtggt | 1080 |
| gacagattgc gtaaaaacat aattggagag gtgcatgatg ctttaaactt ttccgaccac | 1140 |
| gctaacttac aaagattagc tattcgtaga aggattaagc attacgctac tgacgataca | 1200 |
| aggattctaa aaacttccta cagatgctca acaatcggta accaagattt tctaaaactt | 1260 |
| gcagtggaag atttcaatat ctgtcaatca atacaaagag aggaattcaa gcatattgaa | 1320 |
| agatgggtcg ttgaaagacg tctagacaag ttaaagttcg ctagacaaaa agaggcctat | 1380 |
| tgctatttct cagccgcagc aacattgttt gcccctgaat tgtctgatgc tagaatgtct | 1440 |
| tgggccaaaa atggtgtatt gacaactgtg gttgatgatt tcttcgatgt cggaggctct | 1500 |
| gaagaggaat tagttaactt gatagaattg atcgagcgtt gggatgtgaa tggcagtgca | 1560 |
| gatttttgta gtgaggaagt tgagattatc tattctgcta tccactcaac tatctctgaa | 1620 |
| ataggtgata agtcatttgg ctggcaaggt agagatgtaa agtctcaagt tatcaagatc | 1680 |
| tggctggact tattgaaatc aatgttaact gaagctcaat ggtcttcaaa caagtctgtt | 1740 |
| cctaccctag atgagtatat gacaaccgcc catgtttcat tcgcacttgg tccaattgta | 1800 |
| cttccagcct tatacttcgt tggcccaaag ttgtcagaag aggttgcagg tcatcctgaa | 1860 |
| ctactaaacc tctacaaagt cacatctact tgtggcagac tactgaatga ttggagaagt | 1920 |
| tttaagagag aatccgagga aggtaagctc aacgctatta gtttatacat gatccactcc | 1980 |
| ggtggtgctt ctacagaaga ggaaacaatc gaacatttca aaggtttgat tgattctcag | 2040 |
| agaaggcaac tgttacaatt ggtgttgcaa gagaaggata gtatcatacc tagaccatgt | 2100 |
| aaagatctat tttggaatat gattaagtta ttacacactt tctacatgaa agatgatggc | 2160 |
| ttcacctcaa atgagatgag gaatgtagtt aaggcaatca ttaacgaacc aatctcactg | 2220 |
| gatgaattat ga | 2232 |

SEQ ID NO: 50
Populus trichocarpa

| | |
|---|---|
| MSCIRPWFCP SSISATLTDP ASKLVTGEFK TTSLNFHGTK ERIKKMFDKI ELSVSSYDTA | 60 |
| WVAMVPSPDC PETPCFPECT KWILENQLGD GSWSLPHGNP LLVKDALSST LACILALKRW | 120 |
| GIGEEQINKG LRFIELNSAS VTDNEQHKPI GFDIIFPGMI EYAKDLDLNL PLKPTDINSM | 180 |
| LHRRALELTS GGGKNLEGRR AYLAYVSEGI GKLQDWEMAM KYQRKNGSLF NSPSTTAAAF | 240 |
| IHIQDAECLH YIRSLLQKFG NAVPTIYPLD IYARLSMVDA LERLGIDRHF RKERKFVLDE | 300 |
| TYRFWLQGEE EIFSDNATCA LAFRILRLNG YDVSLEDHFS NSLGGYLKDS GAALELYRAL | 360 |
| QLSYPDESLL EKQNSRTSYF LKQGLSNVSL CGDRLRKNII GEVHDALNFP DHANLQRLAI | 420 |
| RRRIKHYATD DTRILKTSYR CSTIGNQDFL KLAVEDFNIC QSIQREEFKH IERWVVERRL | 480 |
| DKLKFARQKE AYCYFSAAAT LFAPELSDAR MSWAKNGVLT TVVDDFFDVG GSEEELVNLI | 540 |
| ELIERWDVNG SADFCSEEVE IIYSAIHSTI SEIGDKSFGW QGRDVKSHVI KIWLDLLKSM | 600 |
| LTEAQWSSNK SVPTLDEYMT TAHVSFALGP IVLPALYFVG PKLSEEVAGH PELLNLYKVM | 660 |
| STCGRLLNDW RSFKRESEEG KLNAISLYMI HSGGASTEEE TIEHFKGLID SQRRQLLQLV | 720 |
| LQEKDSIIPR PCKDLFWNMI KLLHTFYMKD DGFTSNEMRN VVKAIINEPI SLDEL | 775 |

SEQ ID NO: 51
Artificial Sequence

| | |
|---|---|
| atgtctatca accttcgctc ctccggttgt tcgtctccga tctcagctac tttggaacga | 60 |
| ggattggact cagaagtaca gacaagagct aacaatgtga gctttgagca aacaaaggag | 120 |
| aagattagga agatgttgga gaaagtggag ctttctgttt cggcctacga tactagttgg | 180 |
| gtagcaatgg ttccatcacc gagctcccaa aatgctccac ttttcccaca gtgtgtgaaa | 240 |
| tggttattgg ataatcaaca tgaagatgga tcttggggac ttgataacca tgaccatcaa | 300 |
| tctcttaaga aggatgtgtt atcatctaca ctggctagta tcctcgcgtt aaagaagtgg | 360 |
| ggaattggtg aaagacaaat aaacaagggt ctccagttta ttgagctgaa ttctgcatta | 420 |
| gtcactgatg aaaccataca gaaaccaaca gggtttgata ttatatttcc tgggatgatt | 480 |
| aaatatgcta gagatttgaa tctgacgatt ccattgggct cagaagtggt ggatgacatg | 540 |
| atacgaaaaa gagatctgga tcttaaatgt gatagtgaaa agttttcaaa gggaagagaa | 600 |
| gcatatctgg cctatgtttt agaggggaca agaaacctaa aagattggga tttgatagtc | 660 |
| aaatatcaaa ggaaaaatgg gtcactgttt gattctccag ccacaacagc agctgctttt | 720 |
| actcagtttg ggaatgatgg ttgtctccgt tatctctgtt ctctccttca gaaattcgag | 780 |
| gctgcagttc cttcagttta tccatttgat caatatgcac gccttagtat aattgtcact | 840 |
| cttgaaagct taggaattga tagagatttc aaaaccgaaa tcaaaagcat attggatgaa | 900 |
| acctatagat attggcttcg tggggatgaa gaaatatgtt tggacttggc cacttgtgct | 960 |
| ttggctttcc gattattgct tgctcatggc tatgatgtgt cttacgatcc gctaaaacca | 1020 |
| tttgcagaag aatctggttt ctctgatact ttggaaggat atgttaagaa tacgttttct | 1080 |
| gtgttagaat tatttaaggc tgctcaaagt tatccacatg aatcagcttt gaagaagcag | 1140 |
| tgttgttgga ctaaacaata tctggagatg gaattgtcca gctgggttaa gacctctgtt | 1200 |
| cgagataaat acctcaagaa agaggtcgag gatgctcttg cttttccctc ctatgcaagc | 1260 |
| ctagaaagat cagatcacag gagaaaaata ctcaatggtt ctgctgtgga aaacaccaga | 1320 |
| gttacaaaaa cctcatatcg tttgcacaat atttgcacct ctgatatcct gaagttagct | 1380 |
| gtggatgact tcaatttctg ccagtccata caccgtgaag aaatggaacg tcttgatagg | 1440 |
| tggattgtgg agaatagatt gcaggaactg aaatttgcca gacagaagct ggcttactgt | 1500 |
| tatttctctg gggctgcaac tttattttct ccagaactat ctgatgctcg tatatcgtgg | 1560 |
| gccaaaggtg gagtacttac aacggttgta gacgacttct ttgatgttgg agggtccaaa | 1620 |
| gaagaactgg aaaacctcat acacttggtc gaaaagtggg atttgaacgg tgttcctgag | 1680 |
| tacagctcag aacatgttga gatcatattc tcagttctaa gggacaccat tctcgaaaca | 1740 |
| ggagacaaag cattcaccta tcaaggacgc aatgtgacac accacattgt gaaaatttgg | 1800 |
| ttggatctgc tcaagtctat gttgagagaa gccgagtggt ccagtgacaa gtcaacacca | 1860 |
| agcttggagg attacatgga aaatgcgtac atatcatttg cattaggacc aattgtcctc | 1920 |
| ccagctacct atctgatcgg acctccactt ccagagaaga cagtcgatag ccaccaatat | 1980 |
| aatcagctct acaagctcgt gagcactatg ggtcgtcttc taaatgacat acaaggtttt | 2040 |
| aagagagaaa gcgcggaagg gaagctgaat gcggtttcat tgcacatgaa acacgagaga | 2100 |
| gacaatcgca gcaaagaagt gatcatagaa tcgatgaaag gtttagcaga gagaaagagg | 2160 |
| gaagaattgc ataagctagt tttggaggag aaaggaagtg tggttccaag ggaatgcaaa | 2220 |
| gaagcgttct tgaaaatgag caaagtgttg aacttatttt acaggaagga cgatggattc | 2280 |
| acatcaaatg atctgatgag tcttgttaaa tcagtgatct acgagcctgt tagcttacag | 2340 |
| aaagaatctt taacttga | 2358 |

SEQ ID NO: 52
Arabidopsis thaliana

| | |
|---|---|
| MSINLRSSGC SSPISATLER GLDSEVQTRA NNVSFEQTKE KIRKMLEKVE LSVSAYDTSW | 60 |
| VAMVPSPSSQ NAPLFPQCVK WLLDNQHEDG SWGLDNHDHQ SLKKDVLSST LASILALKKW | 120 |
| GIGERQINKG LQFIELNSAL VTDETIQKPT GFDIIFPGMI KYARDLNLTI PLGSEVVDDM | 180 |
| IRKRDLDLKC DSEKFSKGRE AYLAYVLEGT RNLKDWDLIV KYQRKNGSLF DSPATTAAAF | 240 |
| TQFGNDGCLR YLCSLLQKFE AAVPSVYPFD QYARLSIIVT LESLGIDRDF KTEIKSILDE | 300 |
| TYRYWLRGDE EICLDLATCA LAFRLLLAHG YDVSYDPLKP FAEESGFSDT LEGYVKNTFS | 360 |
| VLELFKAAQS YPHESALKKQ CCWTKQYLEM ELSSWVKTSV RDKYLKKEVE DALAFPSYAS | 420 |
| LERSDHRRKI LNGSAVENTR VTKTSYRLHN ICTSDILKLA VDDFNFCQSI HREEMERLDR | 480 |
| WIVENRLQEL KFARQKLAYC YFSGAATLFS PELSDARISW AKGGVLTTVV DDFFDVGGSK | 540 |
| EELENLIHLV EKWDLNGVPE YSSEHVEIIF SVLRDTILET GDKAFTYQGR NVTHHIVKIW | 600 |
| LDLLKSMLRE AEWSSDKSTP SLEDYMENAY ISFALGPIVL PATYLIGPPL PEKTVDSHQY | 660 |
| NQLYKLVSTM GRLLNDIQGF KRESAEGKLN AVSLHMKHER DNRSKEVIIE SMKGLAERKR | 720 |
| EELHKLVLEE KGSVVPRECK EAFLKMSKVL NLFYRKDDGF TSNDLMSLVK SVIYEPVSLQ | 780 |
| KESLT | 785 |

SEQ ID NO: 53
Artificial Sequence

| | |
|---|---|
| atggaatttg atgaaccatt ggttgacgaa gcaagatctt tagtgcagcg tactttacaa | 60 |
| gattatgatg acagatacgg cttcggtact atgtcatgtg ctgcttatga tacagcctgg | 120 |
| gtgtctttag ttacaaaaac agtcgatggg agaaaacaat ggcttttccc agagtgtttt | 180 |
| gaatttctac tagaaacaca atctgatgcc ggaggatggg aaatcgggaa ttcagcacca | 240 |
| atcgacggta tattgaatac agctgcatcc ttacttgctc taaaacgtca cgttcaaact | 300 |
| gagcaaatca tccaacctca acatgaccat aaggatctag caggtagagc tgaacgtgcc | 360 |
| gctgcatctt tgagagcaca attggctgca ttggatgtgt ctacaactga acacgtcggt | 420 |
| tttgagataa ttgttcctgc aatgctagac ccattagaag ccgaagatcc atctctagtt | 480 |
| ttcgattttc cagctaggaa acctttgatg aagattcatg atgctaagat gagtagattc | 540 |
| aggccagaat acttgtatgg caaacaacca atgaccgcct tacattcatt agaggctttc | 600 |
| ataggcaaaa tcgacttcga taaggtaaga caccaccgta cccatgggtc tatgatgggt | 660 |
| tctccttcat ctaccgcagc ctacttaatg cacgcttcac aatgggatgg tgactcagag | 720 |
| gcttacctta gacacgtgat taaacacgca gcagggcagg gaactggtgc tgtaccatct | 780 |
| gctttcccat caacacattt tgagtcatct tggattctta ccacattgtt tagagctgga | 840 |
| ttttcagctt ctcatcttgc ctgtgatgag ttgaacaagt tggtcgagat acttgagggc | 900 |
| tcattcgaga aggaaggtgg ggcaatcggt tacgctccag ggtttcaagc agatgttgat | 960 |
| gatactgcta aaacaataag tacattagca gtccttggaa gagatgctac accaagacaa | 1020 |
| atgatcaagg tatttgaagc taatacacat tttagaacat accctggtga aagagatcct | 1080 |
| tctttgacag ctaattgtaa tgctctatca gccttactac accaaccaga tgcagcaatg | 1140 |
| tatggatctc aaattcaaaa gattaccaaa tttgtctgtg actattggtg gaagtctgat | 1200 |
| ggtaagatta aagataagtg gaacacttgc tacttgtacc catctgtctt attagttgag | 1260 |
| gttttggttg atcttgttag tttattggag cagggtaaat tgcctgatgt tttggatcaa | 1320 |
| gagcttcaat acagagtcgc catcacattg ttccaagcat gtttaaggcc attactagac | 1380 |
| caagatgccg aaggatcatg gaacaagtct atcgaagcca cagcctacgg catccttatc | 1440 |
| ctaactgaag ctaggagagt ttgtttcttc gacagattgt ctgagccatt gaatgaggca | 1500 |
| atccgtagag gtatcgcttt cgccgactct atgtctggaa ctgaagctca gttgaactac | 1560 |
| atttggatcg aaaaggttag ttacgcacct gcattattga ctaaatccta tttgttagca | 1620 |
| gcaagatggg ctgctaagtc tcctttaggc gcttccgtag gctcttcttt gtggactcca | 1680 |
| ccaagagaag gattggataa gcatgtcaga ttattccatc aagctgagtt attcagatcc | 1740 |
| cttccagaat gggaattaag agcctccatg attgaagcag ctttgttcac accacttcta | 1800 |
| agagcacata gactagacgt tttccctaga caagatgtag gtgaagacaa atatcttgat | 1860 |
| gtagttccat tcttttggac tgccgctaac aacagagata gaacttacgc ttccactcta | 1920 |
| ttcctttacg atatgtgttt tatcgcaatg ttaaacttcc agttagacga attcatggag | 1980 |
| gccacagccg gtatcttatt cagagatcat atggatgatt tgaggcaatt gattcatgat | 2040 |
| cttttggcag agaaaacttc cccaaagagt tctggtagaa gtagtcaggg cacaaaagat | 2100 |
| gctgactcag gtatagagga agacgtgtca atgtccgatt cagcttcaga ttcccaggat | 2160 |
| agaagtccag aatacgactt ggttttcagt gcattgagta cctttacaaa acatgtcttg | 2220 |
| caacacccat ctatacaaag tgcctctgta tgggatagaa aactacttgc tagagagatg | 2280 |
| aaggcttact tacttgctca tatccaacaa gcagaagatt caactccatt gtctgaattg | 2340 |
| aaagatgtgc ctcaaaagac tgatgtaaca agagtttcta catctactac taccttcttt | 2400 |
| aactgggtta gaacaacttc cgcagaccat atatcctgcc catactcctt ccactttgta | 2460 |
| gcatgccatc taggcgcagc attgtcacct aaagggtcta acggtgattg ctatccttca | 2520 |
| gctggtgaga agttcttggc agctgcagtc tgcagacatt tggccaccat gtgtagaatg | 2580 |
| tacaacgatc ttggatcagc tgaacgtgat tctgatgaag gtaatttgaa ctccttggac | 2640 |
| ttccctgaat tcgccgattc cgcaggaaac ggagggatag aaattcagaa ggccgctcta | 2700 |
| ttaaggttag ctgagtttga gagagattca tacttagagg ccttccgtcg tttacaagat | 2760 |
| gaatccaata gagttcacgg tccagccggt ggtgatgaag ccagattgtc cagaaggaga | 2820 |
| atggcaatcc ttgaattctt cgcccagcag gtagatttgt acggtcaagt atacgtcatt | 2880 |
| agggatattt ccgctcgtat tcctaaaaac gaggttgaga aaaagagaaa attggatgat | 2940 |
| gctttcaatt ga | 2952 |

SEQ ID NO: 54
Phomopsis amygdali

| | |
|---|---|
| MEFDEPLVDE ARSLVQRTLQ DYDDRYGFGT MSCAAYDTAW VSLVTKTVDG RKQWLFPECF | 60 |
| EFLLETQSDA GGWEIGNSAP IDGILNTAAS LLALKRHVQT EQIIQPQHDH KDLAGRAERA | 120 |
| AASLRAQLAA LDVSTTEHVG FEIIVPAMLD PLEAEDPSLV FDFPARKPLM KIHDAKMSRF | 180 |
| RPEYLYGKQP MTALHSLEAF IGKIDFDKVR HHRTHGSMMG SPSSTAAYLM HASQWDGDSE | 240 |
| AYLRHVIKHA AGQGTGAVPS AFPSTHFESS WILTTLFRAG FSASHLACDE LNKLVEILEG | 300 |
| SFEKEGGAIG YAPGFQADVD DTAKTISTLA VLGRDATPRQ MIKVFEANTH FRTYPGERDP | 360 |
| SLTANCNALS ALLHQPDAAM YGSQIQKITK FVCDYWWKSD GKIKDKWNTC YLYPSVLLVE | 420 |
| VLVDLVSLLE QGKLPDVLDQ ELQYRVAITL FQACLRPLLD QDAEGSWNKS IEATAYGILI | 480 |
| LTEARRVCFF DRLSEPLNEA IRRGIAFADS MSGTEAQLNY IWIEKVSYAP ALLTKSYLLA | 540 |
| ARWAAKSPLG ASVGSSLWTP PREGLDKHVR LFHQAELFRS LPEWELRASM IEAALFTPLL | 600 |
| RAHRLDVFPR QDVGEDKYLD VVPFFWTAAN NRDRTYASTL FLYDMCFIAM LNFQLDEFME | 660 |
| ATAGILFRDH MDDLRQLIHD LLAEKTSPKS SGRSSQGTKD ADSGIEEDVS MSDSASDSQD | 720 |
| RSPEYDLVFS ALSTFTKHVL QHPSIQSASV WDRKLLAREM KAYLLAHIQQ AEDSTPLSEL | 780 |
| KDVPQKTDVT RVSTSTTTFF NWVRTTSADH ISCPYSFHFV ACHLGAALSP KGSNGDCYPS | 840 |
| AGEKFLAAAV CRHLATMCRM YNDLGSAERD SDEGNLNSLD FPEFADSAGN GGIEIQKAAL | 900 |
| LRLAEFERDS YLEAFRRLQD ESNRVHGPAG GDEARLSRRR MAILEFFAQQ VDLYGQVYVI | 960 |
| RDISARIPKN EVEKKRKLDD AFN | 983 |

SEQ ID NO: 55
Artificial Sequence

| | |
|---|---|
| atggcttcta gtacacttat ccaaaacaga tcatgtggcg tcacatcatc tatgtcaagt | 60 |
| tttcaaatct tcagaggtca accactaaga tttcctggca ctagaacccc agctgcagtt | 120 |
| caatgcttga aaaagaggag atgccttagg ccaaccgaat ccgtactaga atcatctcct | 180 |
| ggctctggtt catatagaat agtaactggc ccttctggaa ttaaccctag ttctaacggg | 240 |
| cacttgcaag agggttcctt gactcacagg ttaccaatac caatggaaaa atctatcgat | 300 |
| aacttccaat ctactctata tgtgtcagat atttggtctg aaacactaca gagaactgaa | 360 |
| tgtttgctac aagtaactga aaacgtccag atgaatgagt ggattgagga aattagaatg | 420 |
| tactttagaa atatgacttt aggtgaaatt tccatgtccc cttacgacac tgcttgggtg | 480 |
| gctagagttc cagcgttgga cggttctcat gggcctcaat tccacagatc tttgcaatgg | 540 |
| attatcgaca accaattacc agatggggac tggggcgaac cttctctttt cttgggttac | 600 |
| gatagagttt gtaatacttt agcctgtgtg attgcgttga aaacatgggg tgttggggca | 660 |
| caaaacgttg aaagaggaat tcagttccta caatctaaca tatacaagat ggaggaagat | 720 |
| gacgctaatc atatgccaat aggattcgaa atcgtattcc ctgctatgat ggaagatgcc | 780 |
| aaagcattag gtttggattt gccatacgat gctactattt tgcaacagat ttcagccgaa | 840 |
| agagagaaaa agatgaaaaa gatcccaatg gcaatggtgt acaaataccc aaccacttta | 900 |
| cttcactcct tagaaggctt gcatagagaa gttgattgga ataagttgtt acaattacaa | 960 |
| tctgaaaatg gtagttttct ttattcacct gcttcaaccg catgcgcctt aatgtacact | 1020 |
| aaggacgtta aatgttttga ttacttaaac cagttgttga tcaagttcga ccacgcatgc | 1080 |
| ccaaatgtat atccagtcga tctattcgaa agattatgga tggttgacag attgcagaga | 1140 |
| ttagggatct ccagatactt tgaaagagag attagagatt gtttacaata cgtctacaga | 1200 |
| tattggaaag attgtggaat cggatgggct tctaactctt ccgtacaaga tgttgatgat | 1260 |
| acagccatgg cgtttagact tttaaggact catggtttcg acgtaaagga agattgcttt | 1320 |
| agacagtttt tcaaggacgg agaattcttc tgcttcgcag gccaatcatc tcaagcagtt | 1380 |
| acaggcatgt ttaatctttc aagagccagt caaacattgt ttccaggaga atctttattg | 1440 |
| aaaaaggcta gaaccttctc tagaaacttc ttgagaacaa agcatgagaa caacgaatgt | 1500 |
| ttcgataaat ggatcattac taaagatttg gctggtgaag tcgagtataa cttgaccttc | 1560 |
| ccatggtatg cctctttgcc tagattagaa cataggacat acttagatca atatggaatc | 1620 |
| gatgatatct ggataggcaa atctttatac aaaatgcctg ctgttaccaa cgaagttttc | 1680 |
| ctaaagttgg caaaggcaga ctttaacatg tgtcaagctc tacacaaaaa ggaattggaa | 1740 |
| caagtgataa agtggaacgc gtcctgtcaa ttcagagatc ttgaattcgc cagacaaaaa | 1800 |
| tcagtagaat gctattttgc tggtgcagcc acaatgttcg aaccagaaat ggttcaagct | 1860 |
| agattagtct gggcaagatg ttgtgtattg acaactgtct tagacgatta ctttgaccac | 1920 |
| gggacacctg ttgaggaact tagagtgttt gttcaagctg tcagaacatg gaatccagag | 1980 |
| ttgatcaacg gtttgccaga gcaagctaaa atcttgttta tgggcttata caaaacagtt | 2040 |
| aacacaattg cagaggaagc attcatggca cagaaaagag acgtccatca tcatttgaaa | 2100 |
| cactattggg acaagttgat aacaagtgcc ctaaaggagg ccgaatgggc agagtcaggt | 2160 |
| tacgtcccaa catttgatga atacatggaa gtagctgaaa tttctgttgc tctagaacca | 2220 |
| attgtctgta gtaccttgtt ctttgcgggt catagactag atgaggatgt tctagatagt | 2280 |
| tacgattacc atctagttat gcatttggta aacagagtcg gtagaatctt gaatgatata | 2340 |
| caaggcatga agagggaggc ttcacaaggt aagatctcat cagttcaaat ctacatggag | 2400 |
| gaacatccat ctgttccatc tgaggccatg gcgatcgctc atcttcaaga gttagttgat | 2460 |
| aattcaatgc agcaattgac atacgaagtt cttaggttca ctgcggttcc aaaaagttgt | 2520 |
| aagagaatcc acttgaatat ggctaaaatc atgcatgcct tctacaagga tactgatgga | 2580 |
| ttctcatccc ttactgcaat gacaggattc gtcaaaaagg ttcttttcga acctgtgcct | 2640 |
| gagtaa | 2646 |

SEQ ID NO: 56
Physcomitrella patens

| | |
|---|---|
| MASSTLIQNR SCGVTSSMSS FQIFRGQPLR FPGTRTPAAV QCLKKRRCLR PTESVLESSP | 60 |
| GSGSYRIVTG PSGINPSSNG HLQEGSLTHR LPIPMEKSID NFQSTLYVSD IWSETLQRTE | 120 |
| CLLQVTENVQ MNEWIEEIRM YFRNMTLGEI SMSPYDTAWV ARVPALDGSH GPQFHRSLQW | 180 |
| IIDNQLPDGD WGEPSLFLGY DRVCNTLACV IALKTWGVGA QNVERGIQFL QSNIYKMEED | 240 |
| DANHMPIGFE IVFPAMMEDA KALGLDLPYD ATILQQISAE REKKMKKIPM AMVYKYPTTL | 300 |
| LHSLEGLHRE VDWNKLLQLQ SENGSFLYSP ASTACALMYT KDVKCFDYLN QLLIKFDHAC | 360 |
| PNVYPVDLFE RLWMVDRLQR LGISRYFERE IRDCLQYVYR YWKDCGIGWA SNSSVQDVDD | 420 |
| TAMAFRLLRT HGFDVKEDCF RQFFKDGEFF CFAGQSSQAV TGMFNLSRAS QTLFPGESLL | 480 |
| KKARTFSRNF LRTKHENNEC FDKWIITKDL AGEVEYNLTF PWYASLPRLE HRTYLDQYGI | 540 |
| DDIWIGKSLY KMPAVTNEVF LKLAKADFNM CQALHKKELE QVIKWNASCQ FRDLEFARQK | 600 |
| SVECYFAGAA TMFEPEMVQA RLVWARCCVL TTVLDDYFDH GTPVEELRVF VQAVRTWNPE | 660 |
| LINGLPEQAK ILFMGLYKTV NTIAEEAFMA QKRDVHHHLK HYWDKLITSA LKEAEWAESG | 720 |
| YVPTFDEYME VAEISVALEP IVCSTLFFAG HRLDEDVLDS YDYHLVMHLV NRVGRILNDI | 780 |
| QGMKREASQG KISSVQIYME EHPSVPSEAM AIAHLQELVD NSMQQLTYEV LRFTAVPKSC | 840 |
| KRIHLNMAKI MHAFYKDTDG FSSLTAMTGF VKKVLFEPVP E | 881 |

SEQ ID NO: 57
Artificial Sequence

| | |
|---|---|
| atgcctggta aaattgaaaa tggtacccca aaggacctca agactggaaa tgattttgtt | 60 |
| tctgctgcta agagtttact agatcgagct ttcaaaagtc atcattccta ctacggatta | 120 |
| tgctcaactt catgtcaagt ttatgataca gcttgggttg caatgattcc aaaaacaaga | 180 |
| gataatgtaa aacagtggtt gtttccagaa tgtttccatt acctcttaaa aacacaagcc | 240 |
| gcagatggct catggggttc attgcctaca acacagacag cgggtatcct agatacagcc | 300 |
| tcagctgtgc tggcattatt gtgccacgca caagagcctt tacaaatatt ggatgtatct | 360 |
| ccagatgaaa tggggttgag aatagaacac ggtgtcacat ccttgaaacg tcaattagca | 420 |
| gtttggaatg atgtggagga caccaaccat attggcgtcg agtttatcat accagcctta | 480 |
| ctttccatgc tagaaaagga attagatgtt ccatcttttg aatttccatg taggtccatc | 540 |
| ttagagagaa tgcacgggga gaaattaggt catttcgacc tggaacaagt ttacggcaag | 600 |
| ccaagctcat tgttgcactc attggaagca tttctcggta agctagattt tgatcgacta | 660 |
| tcacatcacc tataccacgg cagtatgatg gcatctccat cttcaacggc tgcttatctt | 720 |
| attggggcta caaaatggga tgacgaagcc gaagattacc taagacatgt aatgcgtaat | 780 |
| ggtgcaggac atgggaatgg aggtatttct ggtacatttc caactactca tttcgaatgt | 840 |
| agctggatta tagcaacgtt gttaaaggtt ggctttactt tgaagcaaat tgacggcgat | 900 |
| ggcttaagag gtttatcaac catcttactt gaggcgcttc gtgatgagaa tggtgtcata | 960 |
| ggctttgccc ctagaacagc agatgtagat gacacagcca aagctctatt ggccttgtca | 1020 |
| ttggtaaacc agccagtgtc acctgatatc atgattaagg tctttgaggg caaagaccat | 1080 |
| tttaccactt ttggttcaga aagagatcca tcattgactt ccaacctgca cgtcctttta | 1140 |
| tctttactta aacaatctaa cttgtctcaa taccatcctc aaatcctcaa aacaacatta | 1200 |
| ttcacttgta gatggtggtg gggttccgat cattgtgtca aagacaaatg gaatttgagt | 1260 |
| cacctatatc caactatgtt gttggttgaa gccttcactg aagtgctcca tctcattgac | 1320 |
| ggtggtgaat tgtctagtct gtttgatgaa tcctttaagt gtaagattgg tcttagcatc | 1380 |
| tttcaagcgg tacttagaat aatcctcacc caagacaacg acggctcttg gagaggatac | 1440 |
| agagaacaga cgtgttacgc aatattggct ttagttcaag cgagacatgt atgctttttc | 1500 |
| actcacatgg ttgacagact gcaatcatgt gttgatcgag gtttctcatg gttgaaatct | 1560 |
| tgctcttttc attctcaaga cctgacttgg acctctaaaa cagcttatga agtgggtttc | 1620 |
| gtagctgaag catataaact agctgcttta caatctgctt ccctggaggt tcctgctgcc | 1680 |
| accattggac attctgtcac gtctgccgtt ccatcaagtg atcttgaaaa atacatgaga | 1740 |
| ttggtgagaa aaactgcgtt attctctcca ctggatgagt ggggtctaat ggcttctatc | 1800 |
| atcgaatctt catttttcgt accattactg caggcacaaa gagttgaaat ataccctaga | 1860 |
| gataatatca aggtggacga agataagtac ttgtctatta tcccattcac atgggtcgga | 1920 |
| tgcaataata ggtctagaac tttcgcaagt aacagatggc tatacgatat gatgtacctt | 1980 |
| tcattactcg gctatcaaac cgacgagtac atggaagctg tagctgggcc agtgtttggg | 2040 |
| gatgtttcct tgttacatca aacaattgat aaggtgattg ataatacaat gggtaacctt | 2100 |
| gcgagagcca atggaacagt acacagtggt aatggacatc agcacgaatc tcctaatata | 2160 |
| ggtcaagtcg aggacacctt gactcgtttc acaaattcag tcttgaatca caaagacgtc | 2220 |
| cttaactcta gctcatctga tcaagatact ttgagaagag agtttagaac attcatgcac | 2280 |
| gctcatataa cacaaatcga agataactca cgattcagta agcaagcctc atccgatgcg | 2340 |
| ttttcctctc ctgaacaatc ttactttcaa tgggtgaact caactggtgg ctcacatgtc | 2400 |
| gcttgcgcct attcatttgc cttctctaat tgcctcatgt ctgcaaattt gttgcagggt | 2460 |
| aaagacgcat ttccaagcgg aacgcaaaag tacttaatct cctctgttat gagacatgcc | 2520 |
| acaaacatgt gtagaatgta taacgacttt ggctctattg ccagagacaa cgctgagaga | 2580 |
| aatgttaata gtattcattt tcctgagttt actctctgta acggaacttc tcaaaaccta | 2640 |
| gatgaaagga aggaaagact tctgaaaatc gcaacttacg aacaagggta tttggataga | 2700 |
| gcactagagg ccttggaaag acagagtaga gatgatgccg gagacagagc tggatctaaa | 2760 |
| gatatgagaa agttgaaaat cgttaagtta ttctgtgatg ttacggactt atacgatcag | 2820 |
| ctctacgtta tcaaagattt gtcatcctct atgaagtaa | 2859 |

SEQ ID NO: 58
Gibberella fujikuroi

| | |
|---|---|
| MPGKIENGTP KDLKTGNDFV SAAKSLLDRA FKSHHSYYGL CSTSCQVYDT AWVAMIPKTR | 60 |
| DNVKQWLFPE CFHYLLKTQA ADGSWGSLPT TQTAGILDTA SAVLALLCHA QEPLQILDVS | 120 |
| PDEMGLRIEH GVTSLKRQLA VWNDVEDTNH IGVEFIIPAL LSMLEKELDV PSFEFPCRSI | 180 |
| LERMHGEKLG HFDLEQVYGK PSSLLHSLEA FLGKLDFDRL SHHLYHGSMM ASPSSTAAYL | 240 |
| IGATKWDDEA EDYLRHVMRN GAGHGNGGIS GTFPTTHFEC SWIIATLLKV GFTLKQIDGD | 300 |
| GLRGLSTILL EALRDENGVI GFAPRTADVD DTAKALLALS LVNQPVSPDI MIKVFEGKDH | 360 |
| FTTFGSERDP SLTSNLHVLL SLLKQSNLSQ YHPQILKTTL FTCRWWWGSD HCVKDKWNLS | 420 |
| HLYPTMLLVE AFTEVLHLID GGELSSLFDE SFKCKIGLSI FQAVLRIILT QDNDGSWRGY | 480 |
| REQTCYAILA LVQARHVCFF THMVDRLQSC VDRGFSWLKS CSFHSQDLTW TSKTAYEVGF | 540 |
| VAEAYKLAAL QSASLEVPAA TIGHSVTSAV PSSDLEKYMR LVRKTALFSP LDEWGLMASI | 600 |
| IESSFFVPLL QAQRVEIYPR DNIKVDEDKY LSIIPFTWVG CNNRSRTFAS NRWLYDMMYL | 660 |
| SLLGYQTDEY MEAVAGPVFG DVSLLHQTID KVIDNTMGNL ARANGTVHSG NGHQHESPNI | 720 |
| GQVEDTLTRF TNSVLNHKDV LNSSSSDQDT LRREFRTFMH AHITQIEDNS RFSKQASSDA | 780 |
| FSSPEQSYFQ WVNSTGGSHV ACAYSFAFSN CLMSANLLQG KDAFPSGTQK YLISSVMRHA | 840 |
| TNMCRMYNDF GSIARDNAER NVNSIHFPEF TLCNGTSQNL DERKERLLKI ATYEQGYLDR | 900 |
| ALEALERQSR DDAGDRAGSK DMRKLKIVKL FCDVTDLYDQ LYVIKDLSSS MK | 952 |

SEQ ID NO: 59
Artificial Sequence

| | |
|---|---|
| atggatgctg tgacgggttt gttaactgtc ccagcaaccg ctataactat tggtggaact | 60 |
| gctgtagcat tggcggtagc gctaatcttt tggtacctga aatcctacac atcagctaga | 120 |
| agatcccaat caaatcatct tccaagagtg cctgaagtcc caggtgttcc attgttagga | 180 |
| aatctgttac aattgaagga gaaaaagcca tacatgactt ttacgagatg ggcagcgaca | 240 |
| tatggaccta tctatagtat caaaactggg gctacaagta tggttgtggt atcatctaat | 300 |
| gagatagcca aggaggcatt ggtgaccaga ttccaatcca tatctacaag gaacttatct | 360 |
| aaagccctga aagtacttac agcagataag acaatggtcg caatgtcaga ttatgatgat | 420 |
| tatcataaaa cagttaagag acacatactg accgccgtct tgggtcctaa tgcacagaaa | 480 |
| aagcatagaa ttcacagaga tatcatgatg gataacatat ctactcaact tcatgaattc | 540 |
| gtgaaaaaca acccagaaca ggaagaggta gaccttagaa aaatctttca atctgagtta | 600 |
| ttcggcttag ctatgagaca agccttagga aaggatgttg aaagtttgta cgttgaagac | 660 |
| ctgaaaatca ctatgaatag agacgaaatc tttcaagtcc ttgttgttga tccaatgatg | 720 |
| ggagcaatcg atgttgattg gagagacttc tttccatacc taaagtgggt cccaaacaaa | 780 |
| aagttcgaaa atactattca acaaatgtac atcagaagag aagctgttat gaaatcttta | 840 |
| atcaaagagc acaaaaagag aatagcgtca ggcgaaaagc taaatagtta tatcgattac | 900 |
| cttttatctg aagctcaaac tttaaccgat cagcaactat tgatgtcctt gtgggaacca | 960 |
| atcattgaat cttcagatac aacaatggtc acaacagaat gggcaatgta cgaattagct | 1020 |
| aaaaacccta aattgcaaga taggttgtac agagacatta agtccgtctg tggatctgaa | 1080 |
| aagataaccg aagagcatct atcacagctg ccttacatta cagctatttt ccacgaaaca | 1140 |
| ctgagaagac actcaccagt tcctatcatt cctctaagac atgtacatga agataccgtt | 1200 |
| ctaggcggct accatgttcc tgctggcaca gaacttgccg ttaacatcta cggttgcaac | 1260 |
| atggacaaaa acgtttggga aaatccagag gaatggaacc cagaaagatt catgaaagag | 1320 |
| aatgagacaa ttgattttca aaagacgatg gccttcggtg gtggtaagag agtttgtgct | 1380 |
| ggttccttgc aagccctttt aactgcatct attgggattg ggagaatggt tcaagagttc | 1440 |
| gaatggaaac tgaaggatat gactcaagag gaagtgaaca cgataggcct aactacacaa | 1500 |
| atgttaagac cattgagagc tattatcaaa cctaggatct aa | 1542 |

SEQ ID NO: 60
Stevia rebaudiana

| | |
|---|---|
| MDAVTGLLTV PATAITIGGT AVALAVALIF WYLKSYTSAR RSQSNHLPRV PEVPGVPLLG | 60 |
| NLLQLKEKKP YMTFTRWAAT YGPIYSIKTG ATSMVVVSSN EIAKEALVTR FQSISTRNLS | 120 |
| KALKVLTADK TMVAMSDYDD YHKTVKRHIL TAVLGPNAQK KHRIHRDIMM DNISTQLHEF | 180 |
| VKNNPEQEEV DLRKIFQSEL FGLAMRQALG KDVESLYVED LKITMNRDEI FQVLVVDPMM | 240 |
| GAIDVDWRDF FPYLKWVPNK KFENTIQQMY IRREAVMKSL IKEHKKRIAS GEKLNSYIDY | 300 |
| LLSEAQTLTD QQLLMSLWEP IIESSDTTMV TTEWAMYELA KNPKLQDRLY RDIKSVCGSE | 360 |
| KITEEHLSQL PYITAIFHET LRRHSPVPII PLRHVHEDTV LGGYHVPAGT ELAVNIYGCN | 420 |
| MDKNVWENPE EWNPERFMKE NETIDFQKTM AFGGGKRVCA GSLQALLTAS IGIGRMVQEF | 480 |
| EWKLKDMTQE EVNTIGLTTQ MLRPLRAIIK PRI | 513 |

SEQ ID NO: 61
Artificial Sequence

| | |
|---|---|
| aagcttacta gtaaaatgga cggtgtcatc gatatgcaaa ccattccatt gagaaccgct | 60 |
| attgctattg gtggtactgc tgttgctttg gttgttgcat tatacttttg gttcttgaga | 120 |
| tcctacgctt ccccatctca tcattctaat catttgccac cagtacctga agttccaggt | 180 |
| gttccagttt tgggtaattt gttgcaattg aaagaaaaaa agccttacat gaccttcacc | 240 |
| aagtgggctg aaatgtatgg tccaatctac tctattagaa ctggtgctac ttccatggtt | 300 |
| gttgtctctt ctaacgaaat cgccaaagaa gttgttgtta ccagattccc atctatctct | 360 |
| accagaaaat tgtcttacgc cttgaaggtt ttgaccgaag ataagtctat ggttgccatg | 420 |
| tctgattatc acgattacca taagaccgtc aagagacata ttttgactgc tgttttgggt | 480 |
| ccaaacgccc aaaaaaagtt tagagcacat agagacacca tgatggaaaa cgtttccaat | 540 |
| gaattgcatg ccttcttcga aaagaaccca aatcaagaag tcaacttgag aaagatcttc | 600 |
| caatcccaat tattcggttt ggctatgaag caagccttgg gtaaagatgt tgaatccatc | 660 |
| tacgttaagg atttggaaac caccatgaag agagaagaaa tcttcgaagt tttggttgtc | 720 |
| gatccaatga tgggtgctat tgaagttgat tggagagact ttttcccata cttgaaatgg | 780 |
| gttccaaaca agtccttcga aaacatcatc catagaatgt acactagaag agaagctgtt | 840 |
| atgaaggcct tgatccaaga acacaagaaa agaattgcct ccggtgaaaa cttgaactcc | 900 |
| tacattgatt acttgttgtc tgaagcccaa accttgaccg ataagcaatt attgatgtct | 960 |
| ttgtgggaac ctattatcga atcttctgat accactatgg ttactactga atgggctatg | 1020 |
| tacgaattgg ctaagaatcc aaacatgcaa gacagattat acgaagaaat ccaatccgtt | 1080 |
| tgcggttccg aaaagattac tgaagaaaac ttgtcccaat tgccatactt gtacgctgtt | 1140 |
| ttccaagaaa ctttgagaaa gcactgtcca gttcctatta tgccattgag atatgttcac | 1200 |
| gaaaacaccg ttttgggtgg ttatcatgtt ccagctggta ctgaagttgc tattaacatc | 1260 |
| tacggttgca acatggataa gaaggtctgg gaaaatccag aagaatggaa tccagaaaga | 1320 |
| ttcttgtccg aaaaagaatc catggacttg tacaaaacta tggcttttgg tggtggtaaa | 1380 |
| agagtttgcg ctggttcttt acaagccatg gttatttctt gcattggtat cggtagattg | 1440 |
| gtccaagatt ttgaatggaa gttgaaggat gatgccgaag aagatgttaa cactttgggt | 1500 |
| ttgactaccc aaaagttgca tccattattg gccttgatta acccaagaaa gtaactcgag | 1560 |
| ccgcgg | 1566 |

SEQ ID NO: 62
Lactuca sativa

| | |
|---|---|
| MDGVIDMQTI PLRTAIAIGG TAVALVVALY FWFLRSYASP SHHSNHLPPV PEVPGVPVLG | 60 |
| NLLQLKEKKP YMTFTKWAEM YGPIYSIRTG ATSMVVVSSN EIAKEVVVTR FPSISTRKLS | 120 |
| YALKVLTEDK SMVAMSDYHD YHKTVKRHIL TAVLGPNAQK KFRAHRDTMM ENVSNELHAF | 180 |
| FEKNPNQEVN LRKIFQSQLF GLAMKQALGK DVESIYVKDL ETTMKREEIF EVLVVDPMMG | 240 |
| AIEVDWRDFF PYLKWVPNKS FENIIHRMYT RREAVMKALI QEHKKRIASG ENLNSYIDYL | 300 |
| LSEAQTLTDK QLLMSLWEPI IESSDTTMVT TEWAMYELAK NPNMQDRLYE EIQSVCGSEK | 360 |
| ITEENLSQLP YLYAVFQETL RKHCPVPIMP LRYVHENTVL GGYHVPAGTE VAINIYGCNM | 420 |
| DKKVWENPEE WNPERFLSEK ESMDLYKTMA FGGGKRVCAG SLQAMVISCI GIGRLVQDFE | 480 |
| WKLKDDAEED VNTLGLTTQK LHPLLALINP RK | 512 |

SEQ ID NO: 63
Rubus suavissimus

| | |
|---|---|
| atggccaccc tccttgagca tttccaagct atgccctttg ccatccctat tgcactggct | 60 |
| gctctgtctt ggctgttcct cttttacatc aaagtttcat tcttttccaa caagagtgct | 120 |
| caggctaagc tccctcctgt gccagtggtt cctgggctgc cggtgattgg gaatttactg | 180 |
| caactcaagg agaagaaacc ctaccagact tttacaaggt gggctgagga gtatggacca | 240 |
| atctattcta tcaggactgg tgcttccacc atggtcgttc tcaataccac ccaagttgca | 300 |
| aaagaggcca tggtgaccag atatttatcc atctcaacca gaaagctatc aaacgcacta | 360 |
| aagattctta ctgctgataa atgtatggtt gcaataagtg actacaacga ttttcacaag | 420 |
| atgataaagc gatacatact ctcaaatgtt cttggaccta gtgctcagaa gcgtcaccgg | 480 |
| agcaacagag ataccttgag agctaatgtc tgcagccgat tgcattctca agtaaagaac | 540 |
| tctcctcgag aagctgtgaa tttcagaaga gtttttgagt gggaactctt tggaattgca | 600 |
| ttgaagcaag cctttggaaa ggacatagaa aagcccattt atgtggagga acttggcact | 660 |
| acactgtcaa gagatgagat ctttaaggtt ctagtgcttg acataatgga gggtgcaatt | 720 |
| gaggttgatt ggagagattt cttcccttac ctgagatgga ttccgaatac gcgcatggaa | 780 |
| acaaaaattc agcgactcta tttccgcagg aaagcagtga tgactgccct gatcaacgag | 840 |
| cagaagaagc gaattgcttc aggagaggaa atcaactgtt atatcgactt cttgcttaag | 900 |
| gaagggaaga cactgacaat ggaccaaata agtatgttgc tttgggagac ggttattgaa | 960 |
| acagcagata ctacaatggt aacgacagaa tgggctatgt atgaagttgc taaagactca | 1020 |
| aagcgtcagg atcgtctcta tcaggaaatc caaaaggttt gtggatcgga gatggttaca | 1080 |
| gaggaatact tgtcccaact gccgtacctg aatgcagttt tccatgaaac gctaaggaag | 1140 |
| cacagtccgg ctgcgttagt tcctttaaga tatgcacatg aagataccca actaggaggt | 1200 |
| tactacattc cagctggaac tgagattgct ataaacatat acgggtgtaa catggacaag | 1260 |
| catcaatggg aaagccctga ggaatggaaa ccggagagat ttttggaccc gaaatttgat | 1320 |
| cctatggatt tgtacaagac catggctttt ggggctggaa agagggtatg tgctggttct | 1380 |
| cttcaggcaa tgttaatagc gtgcccgacg attggtaggc tggtgcagga gtttgagtgg | 1440 |
| aagctgagag atggagaaga agaaaatgta gatactgttg ggctcaccac tcacaaacgc | 1500 |
| tatccaatgc atgcaatcct gaagccaaga agtta | 1535 |

SEQ ID NO: 64
Artificial Sequence

| | |
|---|---|
| atggctacct tgttggaaca ttttcaagct atgccattcg ctattccaat tgctttggct | 60 |
| gctttgtctt ggttgttttt gttctacatc aaggtttctt tcttctccaa caaatccgct | 120 |
| caagctaaat tgccaccagt tccagttgtt ccaggtttgc cagttattgg taatttgttg | 180 |
| caattgaaag aaaagaagcc ataccaaacc ttcactagat gggctgaaga atatggtcca | 240 |
| atctactcta ttagaactgg tgcttctact atggttgtct tgaacactac tcaagttgcc | 300 |
| aaagaagcta tggttaccag atacttgtct atctctacca gaaagttgtc caacgccttg | 360 |
| aaaattttga ccgctgataa gtgcatggtt gccatttctg attacaacga tttccacaag | 420 |
| atgatcaaga gatatatctt gtctaacgtt ttgggtccat ctgcccaaaa aagacataga | 480 |
| tctaacagag ataccttgag agccaacgtt tgttctagat tgcattccca agttaagaac | 540 |
| tctccaagag aagctgtcaa ctttagaaga gttttcgaat gggaattatt cggtatcgct | 600 |
| ttgaaacaag ccttcggtaa ggatattgaa aagccaatct acgtcgaaga attgggtact | 660 |
| actttgtcca gagatgaaat cttcaaggtt ttggtcttgg acattatgga aggtgccatt | 720 |
| gaagttgatt ggagagattt tttcccatac ttgcgttgga ttccaaacac cagaatggaa | 780 |
| actaagatcc aaagattata ctttagaaga aaggccgtta tgaccgcctt gattaacgaa | 840 |
| caaaagaaaa gaattgcctc cggtgaagaa atcaactgct acatcgattt cttgttgaaa | 900 |
| gaaggtaaga ccttgaccat ggaccaaatc tctatgttgt tgtgggaaac cgttattgaa | 960 |
| actgctgata ccacaatggt tactactgaa tgggctatgt acgaagttgc taaggattct | 1020 |
| aaaagacaag acagattata ccaagaaatc caaaaggtct gcggttctga aatggttaca | 1080 |
| gaagaatact tgtcccaatt gccatacttg aatgctgttt tccacgaaac tttgagaaaa | 1140 |
| cattctccag ctgctttggt tccattgaga tatgctcatg aagatactca attgggtggt | 1200 |
| tattacattc cagccggtac tgaaattgcc attaacatct acggttgcaa catggacaaa | 1260 |
| caccaatggg aatctccaga agaatggaag ccagaaagat ttttggatcc taagtttgac | 1320 |
| ccaatggact tgtacaaaac tatggctttt ggtgctggta aaagagtttg cgctggttct | 1380 |
| ttacaagcta tgttgattgc ttgtccaacc atcggtagat tggttcaaga atttgaatgg | 1440 |
| aagttgagag atggtgaaga agaaaacgtt gatactgttg gtttgaccac ccataagaga | 1500 |
| tatccaatgc atgctatttt gaagccaaga tcttaa | 1536 |

SEQ ID NO: 65
Artificial Sequence

| | |
|---|---|
| aagcttacta gtaaaatggc ctccatcacc catttcttac aagattttca agctactcca | 60 |
| ttcgctactg cttttgctgt tggtggtgtt tctttgttga tattcttctt cttcatccgt | 120 |
| ggtttccact ctactaagaa aaacgaatat tacaagttgc caccagttcc agttgttcca | 180 |
| ggtttgccag ttgttggtaa tttgttgcaa ttgaaagaaa agaagccata caagactttc | 240 |
| ttgagatggg ctgaaattca tggtccaatc tactctatta gaactggtgc ttctaccatg | 300 |
| gttgttgtta actctactca tgttgccaaa gaagctatgg ttaccagatt ctcttcaatc | 360 |
| tctaccagaa agttgtccaa ggctttggaa ttattgacct ccaacaaatc tatggttgcc | 420 |
| acctctgatt acaacgaatt tcacaagatg gtcaagaagt acatcttggc cgaattattg | 480 |
| ggtgctaatg ctcaaaagag acacagaatt catagagaca ccttgatcga aaacgtcttg | 540 |
| aacaaattgc atgcccatac caagaattct ccattgcaag ctgttaactt cagaaagatc | 600 |
| ttcgaatctg aattattcgg tttggctatg aagcaagcct tgggttatga tgttgattcc | 660 |
| ttgttcgttg aagaattggg tactaccttg tccagagaag aaatctacaa cgttttggtc | 720 |
| agtgacatgt tgaagggtgc tattgaagtt gattggagag actttttccc atacttgaaa | 780 |
| tggatcccaa acaagtcctt cgaaatgaag attcaaagat tggcctctag aagacaagcc | 840 |
| gttatgaact ctattgtcaa agaacaaaag aagtccattg cctctggtaa gggtgaaaac | 900 |
| tgttacttga attacttgtt gtccgaagct aagactttga ccgaaaagca aatttccatt | 960 |
| ttggcctggg aaaccattat tgaaactgct gatacaactg ttgttaccac tgaatgggct | 1020 |
| atgtacgaat tggctaaaaa cccaaagcaa caagacagat tatacaacga aatccaaaac | 1080 |
| gtctgcggta ctgataagat taccgaagaa catttgtcca agttgcctta cttgtctgct | 1140 |
| gtttttcacg aaaccttgag aaagtattct ccatctccat tggttccatt gagatacgct | 1200 |
| catgaagata ctcaattggg tggttattat gttccagccg gtactgaaat tgctgttaat | 1260 |
| atctacggtt gcaacatgga caagaatcaa tgggaaactc cagaagaatg gaagccagaa | 1320 |
| agatttttgg acgaaaagta cgatccaatg gacatgtaca agactatgtc ttttggttcc | 1380 |
| ggtaaaagag tttgcgctgg ttctttacaa gctagtttga ttgcttgtac ctccatcggt | 1440 |
| agattggttc aagaatttga atggagattg aaagacggtg aagttgaaaa cgttgatacc | 1500 |
| ttgggtttga ctacccataa gttgtatcca atgcaagcta tcttgcaacc tagaaactga | 1560 |
| ctcgagccgc gg | 1572 |

SEQ ID NO: 66
Castanea mollissima

| | |
|---|---|
| MASITHFLQD FQATPFATAF AVGGVSLLIF FFFIRGFHST KKNEYYKLPP VPVVPGLPVV | 60 |
| GNLLQLKEKK PYKTFLRWAE IHGPIYSIRT GASTMVVVNS THVAKEAMVT RFSSISTRKL | 120 |
| SKALELLTSN KSMVATSDYN EFHKMVKKYI LAELLGANAQ KRHRIHRDTL IENVLNKLHA | 180 |
| HTKNSPLQAV NFRKIFESEL FGLAMKQALG YDVDSLFVEE LGTTLSREEI YNVLVSDMLK | 240 |
| GAIEVDWRDF FPYLKWIPNK SFEMKIQRLA SRRQAVMNSI VKEQKKSIAS GKGENCYLNY | 300 |
| LLSEAKTLTE KQISILAWET IIETADTTVV TTEWAMYELA KNPKQQDRLY NEIQNVCGTD | 360 |
| KITEEHLSKL PYLSAVFHET LRKYSPSPLV PLRYAHEDTQ LGGYYVPAGT EIAVNIYGCN | 420 |
| MDKNQWETPE EWKPERFLDE KYDPMDMYKT MSFGSGKRVC AGSLQASLIA CTSIGRLVQE | 480 |
| FEWRLKDGEV ENVDTLGLTT HKLYPMQAIL QPRN | 514 |

SEQ ID NO: 67
Artificial Sequence

| | |
|---|---|
| atgatttcct tgttgttggg ttttgttgtc tcctccttct tgtttatctt cttcttgaaa | 60 |
| aaattgttgt tcttcttcag tcgtcacaaa atgtccgaag tttctagatt gccatctgtt | 120 |
| ccagttccag gttttccatt gattggtaac ttgttgcaat tgaaagaaaa gaagccacac | 180 |
| aagactttca ccaagtggtc tgaattatat ggtccaatct actctatcaa gatgggttcc | 240 |
| tcttctttga tcgtcttgaa ctctattgaa accgccaaag aagctatggt cagtagattc | 300 |
| tcttcaatct ctaccagaaa gttgtctaac gctttgactg ttttgacctg caacaaatct | 360 |
| atggttgcta cctctgatta cgatgacttt cataagttcg tcaagagatg cttgttgaac | 420 |
| ggtttgttgg gtgctaatgc tcaagaaaga aaaagacatt acagagatgc cttgatcgaa | 480 |
| aacgttacct ctaaattgca tgcccatacc agaaatcatc cacaagaacc agttaacttc | 540 |
| agagccattt tcgaacacga attattcggt gttgctttga aacaagcctt cggtaaagat | 600 |
| gtcgaatcca tctatgtaaa agaattgggt gtcaccttgt ccagagatga aattttcaag | 660 |
| gttttggtcc acgacatgat ggaaggtgct attgatgttg attggagaga tttcttccca | 720 |
| tacttgaaat ggatcccaaa caactctttc gaagccagaa ttcaacaaaa gcacaagaga | 780 |
| agattggctg ttatgaacgc cttgatccaa gacagattga atcaaaacga ttccgaatcc | 840 |
| gatgatgact gctacttgaa tttcttgatg tctgaagcta agaccttgac catggaacaa | 900 |
| attgctattt tggtttggga aaccattatc gaaactgctg ataccacttt ggttactact | 960 |
| gaatgggcta tgtacgaatt ggccaaacat caatctgttc aagatagatt attcaaagaa | 1020 |
| atccaatccg tctgcggtgg tgaaaagatc aaagaagaac aattgccaag attgccttac | 1080 |
| gtcaatggtg tttttcacga aaccttgaga aagtattctc cagctccatt ggttccaatt | 1140 |
| agatacgctc atgaagatac ccaaattggt ggttatcata ttccagccgg ttctgaaatt | 1200 |
| gccattaaca tctacggttg caacatggat aagaagagat gggaaagacc tgaagaatgg | 1260 |
| tggccagaaa gatttttgga agatagatac gaatcctccg acttgcataa gactatggct | 1320 |
| tttggtgctg gtaaaagagt ttgtgctggt gctttacaag ctagtttgat ggctggtatt | 1380 |
| gctatcggta gattggttca agaattcgaa tggaagttga gagatggtga agaagaaaac | 1440 |
| gttgatactt acggtttgac ctcccaaaag ttgtatccat tgatggccat tatcaaccca | 1500 |
| agaagatctt aa | 1512 |

SEQ ID NO: 68
Thellungiella halophila

| | |
|---|---|
| MASMISLLLG FVVSSFLFIF FLKKLLFFFS RHKMSEVSRL PSVPVPGFPL IGNLLQLKEK | 60 |
| KPHKTFTKWS ELYGPIYSIK MGSSSLIVLN SIETAKEAMV SRFSSISTRK LSNALTVLTC | 120 |
| NKSMVATSDY DDFHKFVKRC LLNGLLGANA QERKRHYRDA LIENVTSKLH AHTRNHPQEP | 180 |
| VNFRAIFEHE LFGVALKQAF GKDVESIYVK ELGVTLSRDE IFKVLVHDMM EGAIDVDWRD | 240 |
| FFPYLKWIPN NSFEARIQQK HKRRLAVMNA LIQDRLNQND SESDDDCYLN FLMSEAKTLT | 300 |
| MEQIAILVWE TIIETADTTL VTTEWAMYEL AKHQSVQDRL FKEIQSVCGG EKIKEEQLPR | 360 |
| LPYVNGVFHE TLRKYSPAPL VPIRYAHEDT QIGGYHIPAG SEIAINIYGC NMDKKRWERP | 420 |
| EEWWPERFLE DRYESSDLHK TMAFGAGKRV CAGALQASLM AGIAIGRLVQ EFEWKLRDGE | 480 |
| EENVDTYGLT SQKLYPLMAI INPRRS | 506 |

SEQ ID NO: 69
Artificial Sequence

| | |
|---|---|
| aagcttacta gtaaaatgga catgatgggt attgaagctg ttccatttgc tactgctgtt | 60 |
| gttttgggtg gtatttcctt ggttgttttg atcttcatca gaagattcgt ttccaacaga | 120 |
| aagagatccg ttgaaggttt gccaccagtt ccagatattc caggtttacc attgattggt | 180 |
| aacttgttgc aattgaaaga aaagaagcca cataagacct ttgctagatg ggctgaaact | 240 |
| tacggtccaa ttttctctat tagaactggt gcttctacca tgatcgtctt gaattcttct | 300 |
| gaagttgcca aagaagctat ggtcactaga ttctcttcaa tctctaccag aaagttgtcc | 360 |
| aacgccttga agattttgac cttcgataag tgtatggttg ccacctctga ttacaacgat | 420 |
| tttcacaaaa tggtcaaggg tttcatcttg agaaacgttt taggtgctcc agcccaaaaa | 480 |
| agacatagat gtcatagaga taccttgatc gaaaacatct ctaagtactt gcatgcccat | 540 |
| gttaagactt ctccattgga accagttgtc ttgaagaaga ttttcgaatc cgaaattttc | 600 |
| ggtttggctt tgaaacaagc cttgggtaag gatatcgaat ccatctatgt tgaagaattg | 660 |
| ggtactacct tgtccagaga agaaattttt gccgttttgg ttgttgatcc aatggctggt | 720 |
| gctattgaag ttgattggag agattttttc ccatacttgt cctggattcc aaacaagtct | 780 |
| atggaaatga agatccaaag aatggatttt agaagaggtg ctttgatgaa ggccttgatt | 840 |
| ggtgaacaaa agaaaagaat cggttccggt gaagaaaaga actcctacat tgatttcttg | 900 |
| ttgtctgaag ctaccacttt gaccgaaaag caaattgcta tgttgatctg ggaaaccatc | 960 |
| atcgaaattt ccgatacaac tttggttacc tctgaatggg ctatgtacga attggctaaa | 1020 |
| gacccaaata gacaagaaat cttgtacaga gaaatccaca aggtttgcgg ttctaacaag | 1080 |
| ttgactgaag aaaacttgtc caagttgcca tacttgaact ctgttttcca cgaaaccttg | 1140 |
| agaaagtatt ctccagctcc aatggttcca gttagatatg ctcatgaaga tactcaattg | 1200 |
| ggtggttacc atattccagc tggttctcaa attgccatta acatctacgg ttgcaacatg | 1260 |
| aacaaaaagc aatgggaaaa tcctgaagaa tggaagccag aaagattctt ggacgaaaag | 1320 |
| tatgacttga tggacttgca taagactatg gcttttggtg gtggtaaaag agtttgtgct | 1380 |
| ggtgctttac aagcaatgtt gattgcttgc acttccatcg gtagattcgt tcaagaattt | 1440 |
| gaatggaagt tgatgggtgg tgaagaagaa aacgttgata ctgttgcttt gacctcccaa | 1500 |
| aaattgcatc caatgcaagc cattattaag gccagagaat gactcgagcc gcgg | 1554 |

SEQ ID NO: 70
Vitis vinifera

| | |
|---|---|
| MDMMGIEAVP FATAVVLGGI SLVVLIFIRR FVSNRKRSVE GLPPVPDIPG LPLIGNLLQL | 60 |
| KEKKPHKTFA RWAETYGPIF SIRTGASTMI VLNSSEVAKE AMVTRFSSIS TRKLSNALKI | 120 |
| LTFDKCMVAT SDYNDFHKMV KGFILRNVLG APAQKRHRCH RDTLIENISK YLHAHVKTSP | 180 |
| LEPVVLKKIF ESEIFGLALK QALGKDIESI YVEELGTTLS REEIFAVLVV DPMAGAIEVD | 240 |
| WRDFFPYLSW IPNKSMEMKI QRMDFRRGAL MKALIGEQKK RIGSGEEKNS YIDFLLSEAT | 300 |
| TLTEKQIAML IWETIIEISD TTLVTSEWAM YELAKDPNRQ EILYREIHKV CGSNKLTEEN | 360 |
| LSKLPYLNSV FHETLRKYSP APMVPVRYAH EDTQLGGYHI PAGSQIAINI YGCNMNKKQW | 420 |
| ENPEEWKPER FLDEKYDLMD LHKTMAFGGG KRVCAGALQA MLIACTSIGR FVQEFEWKLM | 480 |
| GGEEENVDTV ALTSQKLHPM QAIIKARE | 508 |

SEQ ID NO: 71
Artificial Sequence

| | |
|---|---|
| aagcttaaaa tgagtaagtc taatagtatg aattctacat cacacgaaac cctttttcaa | 60 |
| caattggtct tgggtttgga ccgtatgcca ttgatggatg ttcactggtt gatctacgtt | 120 |
| gctttcggcg catggttatg ttcttatgtg atacatgttt tatcatcttc ctctacagta | 180 |
| aaagtgccag ttgttggata caggtctgta ttcgaaccta catggttgct tagacttaga | 240 |
| ttcgtctggg aaggtggctc tatcataggt caagggtaca ataagtttaa agactctatt | 300 |
| ttccaagtta ggaaattggg aactgatatt gtcattatac cacctaacta tattgatgaa | 360 |
| gtgagaaaat tgtcacagga caagactaga tcagttgaac ctttcattaa tgattttgca | 420 |
| ggtcaataca caagaggcat ggttttcttg caatctgact tacaaaaccg tgttatacaa | 480 |
| caaagactaa ctccaaaatt ggtttccttg accaaggtca tgaaggaaga gttggattat | 540 |
| gctttaacaa aagagatgcc tgatatgaaa aatgacgaat gggtagaagt agatatcagt | 600 |
| agtataatgg tgagattgat ttccaggatc tccgccagag tctttctagg gcctgaacac | 660 |
| tgtcgtaacc aggaatggtt gactactaca gcagaatatt cagaatcact tttcattaca | 720 |
| gggtttatct taagagttgt acctcatatc ttaagaccat tcatcgcccc tctattacct | 780 |
| tcatacagga ctctacttag aaacgtttca agtggtagaa gagtcatcgg tgacatcata | 840 |
| agatctcagc aaggggatgg taacgaagat atactttcct ggatgagaga tgctgccaca | 900 |
| ggagaggaaa agcaaatcga taacattgct cagagaatgt taattctttc tttagcatca | 960 |
| atccacacta ctgcgatgac catgacacat gccatgtacg atctatgtgc ttgccctgag | 1020 |
| tacattgaac cattaagaga tgaagttaaa tctgttgttg gggcttctgg ctgggacaag | 1080 |
| acagcgttaa acagatttca taagttggac tccttcctaa aagagtcaca aagattcaac | 1140 |
| ccagtattct tattgacatt caatagaatc taccatcaat ctatgacctt atcagatggc | 1200 |
| actaacattc catctggaac acgtattgct gttccatcac acgcaatgtt gcaagattct | 1260 |
| gcacatgtcc caggtccaac cccacctact gaatttgatg gattcagata tagtaagata | 1320 |
| cgttctgata gtaactacgc acaaaagtac ctattctcca tgaccgattc ttcaaacatg | 1380 |
| gctttcggat acggcaagta tgcttgtcca ggtagatttt acgcgtctaa tgagatgaaa | 1440 |
| ctaacattag ccattttgtt gctacaattt gagttcaaac taccagatgg taaaggtcgt | 1500 |
| cctagaaata tcactatcga ttctgatatg attccagacc caagagctag actttgcgtc | 1560 |
| agaaaaagat cacttagaga tgaatgaccg cgg | 1593 |

SEQ ID NO: 72
Gibberella fujikuroi

| | |
|---|---|
| MSKSNSMNST SHETLFQQLV LGLDRMPLMD VHWLIYVAFG AWLCSYVIHV LSSSSTVKVP | 60 |
| VVGYRSVFEP TWLLRLRFVW EGGSIIGQGY NKFKDSIFQV RKLGTDIVII PPNYIDEVRK | 120 |
| LSQDKTRSVE PFINDFAGQY TRGMVFLQSD LQNRVIQQRL TPKLVSLTKV MKEELDYALT | 180 |
| KEMPDMKNDE WVEVDISSIM VRLISRISAR VFLGPEHCRN QEWLTTTAEY SESLFITGFI | 240 |
| LRVVPHILRP FIAPLLPSYR TLLRNVSSGR RVIGDIIRSQ QGDGNEDILS WMRDAATGEE | 300 |
| KQIDNIAQRM LILSLASIHT TAMTMTHAMY DLCACPEYIE PLRDEVKSVV GASGWDKTAL | 360 |
| NRFHKLDSFL KESQRFNPVF LLTFNRIYHQ SMTLSDGTNI PSGTRIAVPS HAMLQDSAHV | 420 |
| PGPTPPTEFD GFRYSKIRSD SNYAQKYLFS MTDSSNMAFG YGKYACPGRF YASNEMKLTL | 480 |
| AILLLQFEFK LPDGKGRPRN ITIDSDMIPD PRARLCVRKR SLRDE | 525 |

SEQ ID NO: 73
Artificial Sequence

| | |
|---|---|
| aagcttaaaa tggaagatcc tactgtctta tatgcttgtc ttgccattgc agttgcaact | 60 |
| ttcgttgtta gatggtacag agatccattg agatccatcc caacagttgg tggttccgat | 120 |
| ttgcctattc tatcttacat cggcgcacta agatggacaa gacgtggcag agagatactt | 180 |
| caagagggat atgatggcta cagaggatct acattcaaaa tcgcgatgtt agaccgttgg | 240 |
| atcgtgatcg caaatggtcc taaactagct gatgaagtca gacgtagacc agatgaagag | 300 |
| ttaaacttta tggacggatt aggagcattc gtccaaacta agtacacctt aggtgaagct | 360 |
| attcataacg atccatacca tgtcgatatc ataagagaaa aactaacaag aggccttcca | 420 |
| gccgtgcttc ctgatgtcat tgaagagttg acacttgcgg ttagacagta cattccaaca | 480 |
| gaaggtgatg aatgggtgtc cgtaaactgt tcaaaggccg caagagatat tgttgctaga | 540 |
| gcttctaata gagtctttgt aggtttgcct gcttgcagaa accaaggtta cttagatttg | 600 |
| gcaatagact ttacattgtc tgttgtcaag gatagagcca tcatcaatat gtttccagaa | 660 |
| ttgttgaagc caatagttgg cagagttgta ggtaacgcca ccagaaatgt tcgtagagct | 720 |
| gttccttttg ttgctccatt ggtggaggaa agacgtagac ttatggaaga gtacggtgaa | 780 |
| gactggtctg aaaaacctaa tgatatgtta cagtggataa tggatgaagc tgcatccaga | 840 |
| gatagttcag tgaaggcaat cgcagagaga ttgttaatgg tgaacttcgc ggctattcat | 900 |
| acctcatcaa acactatcac tcatgctttg taccaccttg ccgaaatgcc tgaaactttg | 960 |
| caaccactta gagaagagat cgaaccatta gtcaaagagg agggctggac caaggctgct | 1020 |
| atgggaaaaa tgtggtggtt agattcattt ctaagagaat ctcaaagata caatggcatt | 1080 |
| aacatcgtat ctttaactag aatggctgac aaagatatta cattgagtga tggcacattt | 1140 |
| ttgccaaaag gtactctagt ggccgttcca gcgtattcta ctcatagaga tgatgctgtc | 1200 |
| tacgctgatg ccttagtatt cgatcctttc agattctcac gtatgagagc gagagaaggt | 1260 |
| gaaggtacaa agcaccagtt cgttaatact tcagtcgagt acgttccatt tggtcacgga | 1320 |
| aagcatgctt gtccaggaag attcttcgcc gcaaacgaat tgaaagcaat gttggcttac | 1380 |
| attgttctaa actatgatgt aaagttgcct ggtgacggta aacgtccatt gaacatgtat | 1440 |
| tggggtccaa cagttttgcc tgcaccagca ggccaagtat tgttcagaaa gagacaagtt | 1500 |
| agtctataac cgcgg | 1515 |

SEQ ID NO: 74
Trametes versicolor

| | |
|---|---|
| MEDPTVLYAC LAIAVATFVV RWYRDPLRSI PTVGGSDLPI LSYIGALRWT RRGREILQEG | 60 |
| YDGYRGSTFK IAMLDRWIVI ANGPKLADEV RRRPDEELNF MDGLGAFVQT KYTLGEAIHN | 120 |
| DPYHVDIIRE KLTRGLPAVL PDVIEELTLA VRQYIPTEGD EWVSVNCSKA ARDIVARASN | 180 |
| RVFVGLPACR NQGYLDLAID FTLSVVKDRA IINMFPELLK PIVGRVVGNA TRNVRRAVPF | 240 |
| VAPLVEERRR LMEEYGEDWS EKPNDMLQWI MDEAASRDSS VKAIAERLLM VNFAAIHTSS | 300 |
| NTITHALYHL AEMPETLQPL REEIEPLVKE EGWTKAAMGK MWWLDSFLRE SQRYNGINIV | 360 |
| SLTRMADKDI TLSDGTFLPK GTLVAVPAYS THRDDAVYAD ALVFDPFRFS RMRAREGEGT | 420 |
| KHQFVNTSVE YVPFGHGKHA CPGRFFAANE LKAMLAYIVL NYDVKLPGDG KRPLNMYWGP | 480 |
| TVLPAPAGQV LFRKRQVSL | 499 |

SEQ ID NO: 75
Artificial Sequence

| | |
|---|---|
| atggcatttt tctctatgat ttcaattttg ttgggatttg ttatttcttc tttcatcttc | 60 |
| atctttttct tcaaaaagtt acttagtttt agtaggaaaa acatgtcaga agtttctact | 120 |
| ttgccaagtg ttccagtagt gcctggtttt ccagttattg ggaatttgtt gcaactaaag | 180 |
| gagaaaaagc ctcataaaac tttcactaga tggtcagaga tatatggacc tatctactct | 240 |
| ataaagatgg gttcttcatc tcttattgta ttgaacagta cagaaactgc taaggaagca | 300 |
| atggtcacta gattttcatc aatatctacc agaaaattgt caaacgccct aacagttcta | 360 |
| acctgcgata agtctatggt cgccacttct gattatgatg acttccacaa attagttaag | 420 |
| agatgtttgc taaatggact tcttggtgct aatgctcaaa agagaaaaag acactacaga | 480 |
| gatgctttga ttgaaaatgt gagttccaag ctacatgcac acgctagaga tcatccacaa | 540 |
| gagccagtta actttagagc aattttcgaa cacgaattgt ttggtgtagc attaaagcaa | 600 |
| gccttcggta aagacgtaga atccatatac gtcaaggagt taggcgtaac attatcaaaa | 660 |
| gatgaaatct ttaaggtgct tgtacatgat atgatggagg gtgcaattga tgtagattgg | 720 |
| agagatttct tcccatattt gaaatggatc cctaataagt cttttgaagc taggatacaa | 780 |
| caaaagcaca agagaagact agctgttatg aacgcactta tacaggacag attgaagcaa | 840 |
| aatgggtctg aatcagatga tgattgttac cttaacttct taatgtctga ggctaaaaca | 900 |
| ttgactaagg aacagatcgc aatccttgtc tgggaaacaa tcattgaaac agcagatact | 960 |
| accttagtca caactgaatg ggccatatac gagctagcca aacatccatc tgtgcaagat | 1020 |
| aggttgtgta aggagatcca gaacgtgtgt ggtggagaga aattcaagga agagcagttg | 1080 |
| tcacaagttc cttaccttaa cggcgttttc catgaaacct tgagaaaata ctcacctgca | 1140 |
| ccattagttc ctattagata cgcccacgaa gatacacaaa tcggtggcta ccatgttcca | 1200 |
| gctgggtccg aaattgctat aaacatctac gggtgcaaca tggacaaaaa gagatgggaa | 1260 |
| agaccagaag attggtggcc agaaagattc ttagatgatg gcaaatatga aacatctgat | 1320 |
| ttgcataaaa caatggcttt cggagctggc aaaagagtgt gtgccggtgc tctacaagcc | 1380 |
| tccctaatgg ctggtatcgc tattggtaga ttggtccaag agttcgaatg gaaacttaga | 1440 |
| gatggtgaag aggaaaatgt cgatacttat gggttaacat ctcaaaagtt atacccacta | 1500 |
| atggcaatca tcaatcctag aagatcctaa | 1530 |

SEQ ID NO: 76
Arabidopsis thaliana

| | |
|---|---|
| MAFFSMISIL LGFVISSFIF IFFFKKLLSF SRKNMSEVST LPSVPVVPGF PVIGNLLQLK | 60 |
| EKKPHKTFTR WSEIYGPIYS IKMGSSSLIV LNSTETAKEA MVTRFSSIST RKLSNALTVL | 120 |
| TCDKSMVATS DYDDFHKLVK RCLLNGLLGA NAQKRKRHYR DALIENVSSK LHAHARDHPQ | 180 |
| EPVNFRAIFE HELFGVALKQ AFGKDVESIY VKELGVTLSK DEIFKVLVHD MMEGAIDVDW | 240 |
| RDFFPYLKWI PNKSFEARIQ QKHKRRLAVM NALIQDRLKQ NGSESDDDCY LNFLMSEAKT | 300 |
| LTKEQIAILV WETIIETADT TLVTTEWAIY ELAKHPSVQD RLCKEIQNVC GGEKFKEEQL | 360 |
| SQVPYLNGVF HETLRKYSPA PLVPIRYAHE DTQIGGYHVP AGSEIAINIY GCNMDKKRWE | 420 |
| RPEDWWPERF LDDGKYETSD LHKTMAFGAG KRVCAGALQA SLMAGIAIGR LVQEFEWKLR | 480 |
| DGEEENVDTY GLTSQKLYPL MAIINPRRS | 509 |

SEQ ID NO: 77
Artificial Sequence

| | |
|---|---|
| atgcaatcag attcagtcaa agtctctcca tttgatttgg tttccgctgc tatgaatggc | 60 |
| aaggcaatgg aaaagttgaa cgctagtgaa tctgaagatc caacaacatt gcctgcacta | 120 |
| aagatgctag ttgaaaatag agaattgttg acactgttca caacttcctt cgcagttctt | 180 |
| attgggtgtc ttgtatttct aatgtggaga cgttcatcct ctaaaaagct ggtacaagat | 240 |
| ccagttccac aagttatcgt tgtaaagaag aaagagaagg agtcagaggt tgatgacggg | 300 |
| aaaaagaaag tttctatttt ctacggcaca caaacaggaa ctgccgaagg ttttgctaaa | 360 |
| gcattagtcg aggaagcaaa agtgagatat gaaaagacct ctttcaaggt tatcgatcta | 420 |
| gatgactacg ctgcagatga tgatgaatat gaggaaaaac tgaaaaagga atccttagcc | 480 |
| ttcttcttct tggccacata cggtgatggt gaacctactg ataatgctgc taacttctac | 540 |
| aagtggttca cagaaggcga cgataaaggt gaatggctga aaaagttaca atacggagta | 600 |
| tttggtttag gtaacagaca atatgaacat ttcaacaaga tcgctattgt agttgatgat | 660 |
| aaacttactg aaatgggagc caaaagatta gtaccagtag gattagggga tgatgatcag | 720 |
| tgtatagaag atgacttcac cgcctggaag gaattggtat ggccagaatt ggatcaactt | 780 |
| ttaagggacg aagatgatac ttctgtgact accccataca ctgcagccgt attggagtac | 840 |
| agagtggttt accatgataa accagcagac tcatatgctg aagatcaaac ccatacaaac | 900 |
| ggtcatgttg ttcatgatgc acagcatcct tcaagatcta atgtggcttt caaaaaggaa | 960 |
| ctacacacct ctcaatcaga taggtcttgt actcacttag aattcgatat ttctcacaca | 1020 |
| ggactgtctt acgaaactgg cgatcacgtt ggcgtttatt ccgagaactt gtccgaagtt | 1080 |
| gtcgatgaag cactaaaact gttagggtta tcaccagaca catacttctc agtccatgct | 1140 |
| gataaggagg atgggacacc tatcggtggt gcttcactac caccaccttt tcctccttgc | 1200 |
| acattgagag acgctctaac cagatacgca gatgtcttat cctcacctaa aaaggtagct | 1260 |
| ttgctggcat tggctgctca tgctagtgat cctagtgaag ccgataggtt aaagttcctg | 1320 |
| gcttcaccag ccggaaaaga tgaatatgca caatggatcg tcgccaacca acgttctttg | 1380 |
| ctagaagtga tgcaaagttt tccatctgcc aagcctccat taggtgtgtt cttcgcagca | 1440 |
| gtagctccac gtttacaacc aagatactac tctatcagtt catctcctaa gatgtctcct | 1500 |
| aacagaatac atgttacatg tgctttggtg tacgagacta ctccagcagg cagaattcac | 1560 |
| agaggattgt gttcaacctg gatgaaaaat gctgtccctt taacagagtc acctgattgc | 1620 |
| tctcaagcat ccattttcgt tagaacatca aatttcagac ttccagtgga tccaaaagtt | 1680 |
| ccagtcatta tgataggacc aggcactggt cttgccccat tcaggggctt tcttcaagag | 1740 |
| agattggcct tgaaggaatc tggtacagaa ttgggttctt ctatcttttt ctttggttgc | 1800 |
| cgtaatagaa aagttgactt tatctacgag gacgagctta acaattttgt tgagacagga | 1860 |
| gcattgtcag aattgatcgt cgcattttca agagaaggga ctgccaaaga gtacgttcag | 1920 |
| cacaagatga gtcaaaaagc ctccgatata tggaaacttc taagtgaagg tgcctatctt | 1980 |
| tatgtctgtg gcgatgcaaa gggcatggcc aaggatgtcc atagaactct gcatacaatt | 2040 |
| gttcaggaac aagggagtct ggattcttcc aaggctgaat tgtacgtcaa aaacttacag | 2100 |
| atgtctggaa gatacttaag agatgtttgg taa | 2133 |

SEQ ID NO: 78
Stevia rebaudiana

| | |
|---|---|
| MQSDSVKVSP FDLVSAAMNG KAMEKLNASE SEDPTTLPAL KMLVENRELL TLFTTSFAVL | 60 |
| IGCLVFLMWR RSSSKKLVQD PVPQVIVVKK KEKESEVDDG KKKVSIFYGT QTGTAEGFAK | 120 |
| ALVEEAKVRY EKTSFKVIDL DDYAADDDEY EEKLKKESLA FFFLATYGDG EPTDNAANFY | 180 |
| KWFTEGDDKG EWLKKLQYGV FGLGNRQYEH FNKIAIVVDD KLTEMGAKRL VPVGLGDDDQ | 240 |
| CIEDDFTAWK ELVWPELDQL LRDEDDTSVT TPYTAAVLEY RVVYHDKPAD SYAEDQTHTN | 300 |
| GHVVHDAQHP SRSNVAFKKE LHTSQSDRSC THLEFDISHT GLSYETGDHV GVYSENLSEV | 360 |
| VDEALKLLGL SPDTYFSVHA DKEDGTPIGG ASLPPPFPPC TLRDALTRYA DVLSSPKKVA | 420 |
| LLALAAHASD PSEADRLKFL ASPAGKDEYA QWIVANQRSL LEVMQSFPSA KPPLGVFFAA | 480 |
| VAPRLQPRYY SISSSPKMSP NRIHVTCALV YETTPAGRIH RGLCSTWMKN AVPLTESPDC | 540 |
| SQASIFVRTS NFRLPVDPKV PVIMIGPGTG LAPFRGFLQE RLALKESGTE LGSSIFFFGC | 600 |
| RNRKVDFIYE DELNNFVETG ALSELIVAFS REGTAKEYVQ HKMSQKASDI WKLLSEGAYL | 660 |
| YVCGDAKGMA KDVHRTLHTI VQEQGSLDSS KAELYVKNLQ MSGRYLRDVW | 710 |

SEQ ID NO: 79
Siraitia grosvenorii

| | |
|---|---|
| atgaaggtca gtccattcga attcatgtcc gctattatca agggtagaat ggacccatct | 60 |
| aactcctcat ttgaatctac tggtgaagtt gcctccgtta tctttgaaaa cagagaattg | 120 |
| gttgccatct tgaccacttc tattgctgtt atgattggtt gcttcgttgt cttgatgtgg | 180 |
| agaagagctg gttctagaaa ggttaagaat gtcgaattgc caaagccatt gattgtccat | 240 |
| gaaccagaac ctgaagttga agatggtaag aagaaggttt ccatcttctt cggtactcaa | 300 |
| actggtactg ctgaaggttt tgctaaggct ttggctgatg aagctaaagc tagatacgaa | 360 |
| aaggctacct tcagagttgt tgatttggat gattatgctg ccgatgatga ccaatacgaa | 420 |
| gaaaaattga agaacgaatc cttcgccgtt ttcttgttgg ctacttatgg tgatggtgaa | 480 |
| cctactgata atgctgctag attttacaag tggttcgccg aaggtaaaga aagaggtgaa | 540 |
| tggttgcaaa acttgcacta tgctgttttt ggtttgggta acagacaata cgaacacttc | 600 |
| aacaagattg ctaaggttgc cgacgaatta ttggaagctc aaggtggtaa tagattggtt | 660 |
| aaggttggtt taggtgatga cgatcaatgc atcgaagatg atttttctgc ttggagagaa | 720 |
| tctttgtggc cagaattgga tatgttgttg agagatgaag atgatgctac tactgttact | 780 |
| actccatata ctgctgctgt cttggaatac agagttgtct ttcatgattc tgctgatgtt | 840 |
| gctgctgaag ataagtcttg gattaacgct aatggtcatg ctgttcatga tgctcaacat | 900 |
| ccattcagat ctaacgttgt cgtcagaaaa gaattgcata cttctgcctc tgatagatcc | 960 |
| tgttctcatt tggaattcaa catttccggt tccgctttga attacgaaac tggtgatcat | 1020 |
| gttggtgtct actgtgaaaa cttgactgaa actgttgatg aagccttgaa cttgttgggt | 1080 |
| ttgtctccag aaacttactt ctctatctac accgataacg aagatggtac tccattgggt | 1140 |
| ggttcttcat tgccaccacc atttccatca tgtactttga gaactgcttt gaccagatac | 1200 |
| gctgatttgt tgaactctcc aaaaaagtct gctttgttgg ctttagctgc tcatgcttct | 1260 |
| aatccagttg aagctgatag attgagatac ttggcttctc cagctggtaa agatgaatat | 1320 |
| gcccaatctg ttatcggttc ccaaaagtct ttgttggaag ttatggctga attcccatct | 1380 |
| gctaaaccac cattaggtgt tttttttgct gctgttgctc caagattgca acctagattc | 1440 |
| tactccattt catcctctcc aagaatggct ccatctagaa tccatgttac ttgtgctttg | 1500 |
| gtttacgata agatgccaac tggtagaatt cataagggtg tttgttctac ctggatgaag | 1560 |
| aattctgttc caatggaaaa gtcccatgaa tgttcttggg ctccaatttt cgttagacaa | 1620 |
| tccaatttta agttgccagc cgaatccaag gttccaatta tcatggttgg tccaggtact | 1680 |
| ggtttggctc cttttagagg ttttttacaa gaaagattgg ccttgaaaga atccggtgtt | 1740 |
| gaattgggtc catccatttt gtttttcggt tgcagaaaca gaagaatgga ttacatctac | 1800 |
| gaagatgaat tgaacaactt cgttgaaacc ggtgctttgt ccgaattggt tattgctttt | 1860 |
| tctagagaag gtcctaccaa agaatacgtc caacataaga tggctgaaaa ggcttctgat | 1920 |
| atctggaact tgatttctga aggtgcttac ttgtacgttt gtggtgatgc taaaggtatg | 1980 |
| gctaaggatg ttcatagaac cttgcatacc atcatgcaag aacaaggttc tttggattct | 2040 |
| tccaaagctg aatccatggt caagaacttg caaatgaatg gtagatactt aagagatgtt | 2100 |
| tggtaa | 2106 |

SEQ ID NO: 80
Siraitia grosvenorii

| | |
|---|---|
| MKVSPFEFMS AIIKGRMDPS NSSFESTGEV ASVIFENREL VAILTTSIAV MIGCFVVLMW | 60 |
| RRAGSRKVKN VELPKPLIVH EPEPEVEDGK KKVSIFFGTQ TGTAEGFAKA LADEAKARYE | 120 |
| KATFRVVDLD DYAADDDQYE EKLKNESFAV FLLATYGDGE PTDNAARFYK WFAEGKERGE | 180 |
| WLQNLHYAVF GLGNRQYEHF NKIAKVADEL LEAQGGNRLV KVGLGDDDQC IEDDFSAWRE | 240 |
| SLWPELDMLL RDEDDATTVT TPYTAAVLEY RVVFHDSADV AAEDKSWINA NGHAVHDAQH | 300 |
| PFRSNVVVRK ELHTSASDRS CSHLEFNISG SALNYETGDH VGVYCENLTE TVDEALNLLG | 360 |
| LSPETYFSIY TDNEDGTPLG GSSLPPPFPS CTLRTALTRY ADLLNSPKKS ALLALAAHAS | 420 |
| NPVEADRLRY LASPAGKDEY AQSVIGSQKS LLEVMAEFPS AKPPLGVFFA AVAPRLQPRF | 480 |
| YSISSSPRMA PSRIHVTCAL VYDKMPTGRI HKGVCSTWMK NSVPMEKSHE CSWAPIFVRQ | 540 |
| SNFKLPAESK VPIIMVGPGT GLAPFRGFLQ ERLALKESGV ELGPSILFFG CRNRRMDYIY | 600 |
| EDELNNFVET GALSELVIAF SREGPTKEYV QHKMAEKASD IWNLISEGAY LYVCGDAKGM | 660 |
| AKDVHRTLHT IMQEQGSLDS SKAESMVKNL QMNGRYLRDV W | 701 |

SEQ ID NO: 81
Artificial Sequence

| | |
|---|---|
| atggcagaat tagatacact tgatatagta gtattaggtg ttatcttttt gggtactgtg | 60 |
| gcatacttta ctaagggtaa attgtggggt gttaccaagg atccatacgc taacggattc | 120 |
| gctgcaggtg gtgcttccaa gcctggcaga actagaaaca tcgtcgaagc tatggaggaa | 180 |
| tcaggtaaaa actgtgttgt tttctacggc agtcaaacag gtacagcgga ggattacgca | 240 |
| tcaagacttg caaaggaagg aaagtccaga ttcggtttga acactatgat cgccgatcta | 300 |
| gaagattatg acttcgataa cttagacact gttccatctg ataacatcgt tatgtttgta | 360 |
| ttggctactt acggtgaagg cgaaccaaca gataacgccg tggatttcta tgagttcatt | 420 |
| actggcgaag atgcctcttt caatgagggc aacgatcctc cactaggtaa cttgaattac | 480 |
| gttgcgttcg gtctgggcaa caatacctac gaacactaca actcaatggt caggaacgtt | 540 |
| aacaaggctc tagaaaagtt aggagctcat agaattggag aagcaggtga gggtgacgac | 600 |
| ggagctggaa ctatggaaga ggacttttta gcttggaaag atccaatgtg ggaagccttg | 660 |
| gctaaaaaga tgggcttgga ggaaagagaa gctgtatatg aacctatttt cgctatcaat | 720 |
| gagagagatg atttgacccc tgaagcgaat gaggtatact tgggagaacc taataagcta | 780 |
| cacttggaag gtacagcgaa aggtccattc aactcccaca acccatatat cgcaccaatt | 840 |
| gcagaatcat acgaactttt ctcagctaag gatagaaatt gtctgcatat ggaaattgat | 900 |
| atttctggta gtaatctaaa gtatgaaaca ggcgaccata tcgcgatctg gcctaccaac | 960 |
| ccaggtgaag aggtcaacaa atttcttgac attctagatc tgtctggtaa gcaacattcc | 1020 |
| gtcgtaacag tgaaagcctt agaacctaca gccaaagttc cttttccaaa tccaactacc | 1080 |
| tacgatgcta tattgagata ccatctggaa atatgcgctc cagtttctag acagtttgtc | 1140 |
| tcaactttag cagcattcgc ccctaatgat gatatcaaag ctgagatgaa ccgtttggga | 1200 |
| tcagacaaag attacttcca cgaaaagaca ggaccacatt actacaatat cgctagattt | 1260 |
| ttggcctcag tctctaaagg tgaaaaatgg acaaagatac cattttctgc tttcatagaa | 1320 |
| ggccttacaa aactacaacc aagatactat tctatctctt cctctagttt agttcagcct | 1380 |
| aaaaagatta gtattactgc tgttgtcgaa tctcagcaaa ttccaggtag agatgaccca | 1440 |
| ttcagaggtg tagcgactaa ctacttgttc gctttgaagc agaaacaaaa cggtgatcca | 1500 |
| aatccagctc cttttggcca atcatacgag ttgacaggac caaggaataa gtatgatggt | 1560 |
| atacatgttc cagtccatgt aagacattct aactttaagc taccatctga tccaggcaaa | 1620 |
| cctattatca tgatcggtcc aggtaccggt gttgcccctt ttagaggctt cgtccaagag | 1680 |
| agggcaaaac aagccagaga tggtgtagaa gttggtaaaa cactgctgtt ctttggatgt | 1740 |
| agaaagagta cagaagattt catgtatcaa aaagagtggc aagagtacaa ggaagctctt | 1800 |
| ggcgacaaat tcgaaatgat tacagctttt tcaagagaag gatctaaaaa ggtttatgtt | 1860 |
| caacacagac tgaaggaaag atcaaaggaa gtttctgatc ttctatccca aaaagcatac | 1920 |
| ttctacgttt gcggagacgc cgcacatatg gcacgtgaag tgaacactgt gttagcacag | 1980 |
| atcatagcag aaggccgtgg tgtatcagaa gccaagggtg aggaaattgt caaaaacatg | 2040 |
| agatcagcaa atcaatacca agtgtgttct gatttcgtaa ctttacactg taaagagaca | 2100 |
| acatacgcga attcagaatt gcaagaggat gtctggagtt aa | 2142 |

SEQ ID NO: 82
Gibberella fujikuroi

| | |
|---|---|
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQVCS DFVTLHCKET TYANSELQED VWS | 713 |

SEQ ID NO: 83
Stevia rebaudiana

| | |
|---|---|
| atgcaatcgg aatccgttga agcatcgacg attgatttga tgactgctgt tttgaaggac | 60 |
| acagtgatcg atacagcgaa cgcatctgat aacggagact caaagatgcc gccggcgttg | 120 |
| gcgatgatgt tcgaaattcg tgatctgttg ctgattttga ctacgtcagt tgctgttttg | 180 |
| gtcggatgtt tcgttgtttt ggtgtggaag agatcgtccg ggaagaagtc cggcaaggaa | 240 |
| ttggagccgc cgaagatcgt tgtgccgaag aggcggctgg agcaggaggt tgatgatggt | 300 |
| aagaagaagg ttacgatttt cttcggaaca caaactggaa cggctgaagg tttcgctaag | 360 |
| gcacttttcg aagaagcgaa agcgcgatat gaaaaggcag cgtttaaagt gattgatttg | 420 |
| gatgattatg ctgctgattt ggatgagtat gcagagaagc tgaagaagga aacatatgct | 480 |
| ttcttcttct tggctacata tggagatggt gagccaactg ataatgctgc caaattttat | 540 |
| aaatggttta ctgagggaga cgagaaaggc gtttggcttc aaaaacttca atatggagta | 600 |
| tttggtcttg gcaacagaca atatgaacat ttcaacaaga ttggaatagt ggttgatgat | 660 |
| ggtctcaccg agcagggtgc aaaacgcatt gttcccgttg gtcttggaga cgacgatcaa | 720 |
| tcaattgaag acgatttttc ggcatggaaa gagttagtgt ggcccgaatt ggatctattg | 780 |
| cttcgcgatg aagatgacaa agctgctgca actccttaca cagctgcaat ccctgaatac | 840 |
| cgcgtcgtat ttcatgacaa acccgatgcg ttttctgatg atcatactca aaccaatggt | 900 |
| catgctgttc atgatgctca acatccatgc agatccaatg tggctgttaa aaaagagctt | 960 |
| catactcctg aatccgatcg ttcatgcaca catcttgaat ttgacatttc tcacactgga | 1020 |
| ttatcttatg aaactgggga tcatgttggt gtatactgtg aaaacctaat tgaagtagtg | 1080 |
| gaagaagctg ggaaattgtt aggattatca acagatactt atttctcgtt acatattgat | 1140 |
| aacgaagatg gttcaccact tggtggacct tcattacaac ctccttttcc tccttgtact | 1200 |
| ttaagaaaag cattgactaa ttatgcagat ctgttaagct ctcccaaaaa gtcaactttg | 1260 |
| cttgctctag ctgctcatgc ttccgatccc actgaagctg atcgtttaag atttcttgca | 1320 |
| tctcgcgagg gcaaggatga atatgctgaa tgggttgttg caaaccaaag aagtcttctt | 1380 |
| gaagtcatgg aagctttccc gtcagctaga ccgccacttg gtgttttctt tgcagcggtt | 1440 |
| gcaccgcgtt tacagcctcg ttactactct atttcttcct ccccaaagat ggaaccaaac | 1500 |
| aggattcatg ttacttgcgc gttggtttat gaaaaaactc ccgcaggtcg tatccacaaa | 1560 |
| ggaatctgct caacctggat gaagaacgct gtacctttga ccgaaagtca agattgcagt | 1620 |
| tgggcaccga tttttgttag aacatcaaac ttcagacttc caattgaccc gaaagtcccg | 1680 |
| gttatcatga ttggtcctgg aaccgggttg gctccattta ggggttttct tcaagaaaga | 1740 |
| ttggctctta aagaatccgg aaccgaactc gggtcatcta ttttattctt cggttgtaga | 1800 |
| aaccgcaaag tggattacat atatgagaat gaactcaaca actttgttga aaatggtgcg | 1860 |
| ctttctgagc ttgatgttgc tttctcccgc gatggcccga cgaaagaata cgtgcaacat | 1920 |
| aaaatgaccc aaaaggcttc tgaaatatgg aatatgcttt ctgagggagc atatttatat | 1980 |
| gtatgtggtg atgctaaagg catggctaaa gatgtacacc gtacacttca caccattgtg | 2040 |
| caagaacagg gaagtttgga ctcgtctaaa gcggagttgt atgtgaagaa tctacaaatg | 2100 |
| tcaggaagat acctccgtga tgtttggtaa | 2130 |

SEQ ID NO: 84
Stevia rebaudiana

| | |
|---|---|
| MQSESVEAST IDLMTAVLKD TVIDTANASD NGDSKMPPAL AMMFEIRDLL LILTTSVAVL | 60 |
| VGCFVVLVWK RSSGKKSGKE LEPPKIVVPK RRLEQEVDDG KKKVTIFFGT QTGTAEGFAK | 120 |
| ALFEEAKARY EKAAFKVIDL DDYAADLDEY AEKLKKETYA FFFLATYGDG EPTDNAAKFY | 180 |
| KWFTEGDEKG VWLQKLQYGV FGLGNRQYEH FNKIGIVVDD GLTEQGAKRI VPVGLGDDDQ | 240 |
| SIEDDFSAWK ELVWPELDLL LRDEDDKAAA TPYTAAIPEY RVVFHDKPDA FSDDHTQTNG | 300 |
| HAVHDAQHPC RSNVAVKKEL HTPESDRSCT HLEFDISHTG LSYETGDHVG VYCENLIEVV | 360 |
| EEAGKLLGLS TDTYFSLHID NEDGSPLGGP SLQPPFPPCT LRKALTNYAD LLSSPKKSTL | 420 |
| LALAAHASDP TEADRLRFLA SREGKDEYAE WVVANQRSLL EVMEAFPSAR PPLGVFFAAV | 480 |
| APRLQPRYYS ISSSPKMEPN RIHVTCALVY EKTPAGRIHK GICSTWMKNA VPLTESQDCS | 540 |
| WAPIFVRTSN FRLPIDPKVP VIMIGPGTGL APFRGFLQER LALKESGTEL GSSILFFGCR | 600 |
| NRKVDYIYEN ELNNFVENGA LSELDVAFSR DGPTKEYVQH KMTQKASEIW NMLSEGAYLY | 660 |
| VCGDAKGMAK DVHRTLHTIV QEQGSLDSSK AELYVKNLQM SGRYLRDVW | 709 |

SEQ ID NO: 85
Artificial Sequence

| | |
|---|---|
| atgcaatcta actccgtgaa gatttcgccg cttgatctgg taactgcgct gtttagcggc | 60 |
| aaggttttgg acacatcgaa cgcatcggaa tcgggagaat ctgctatgct gccgactata | 120 |
| gcgatgatta tggagaatcg tgagctgttg atgatactca caacgtcggt tgctgtattg | 180 |
| atcggatgcg ttgtcgtttt ggtgtggcgg agatcgtcta cgaagaagtc ggcgttggag | 240 |
| ccaccggtga ttgtggttcc gaagagagtg caagaggagg aagttgatga tggtaagaag | 300 |
| aaagttacgg ttttcttcgg cacccaaact ggaacagctg aaggcttcgc taaggcactt | 360 |
| gttgaggaag ctaaagctcg atatgaaaag gctgtcttta aagtaattga tttggatgat | 420 |
| tatgctgctg atgacgatga gtatgaggag aaactaaaga aagaatcttt ggcctttttc | 480 |
| tttttggcta cgtatggaga tggtgagcca acagataatg ctgccagatt ttataaatgg | 540 |
| tttactgagg gagatgcgaa aggagaatgg cttaataagc ttcaatatgg agtatttggt | 600 |
| ttgggtaaca gacaatatga acattttaac aagatcgcaa aagtggttga tgatggtctt | 660 |
| gtagaacagg gtgcaaagcg tcttgttcct gttggacttg gagatgatga tcaatgtatt | 720 |
| gaagatgact tcaccgcatg gaaagagtta gtatggccgg agttggatca attacttcgt | 780 |
| gatgaggatg acacaactgt tgctactcca tacacagctg ctgttgcaga atatcgcgtt | 840 |
| gtttttcatg aaaaaccaga cgcgctttct gaagattata gttatacaaa tggccatgct | 900 |
| gttcatgatg ctcaacatcc atgcagatcc aacgtggctg tcaaaaagga acttcatagt | 960 |
| cctgaatctg accggtcttg cactcatctt gaatttgaca tctcgaacac cggactatca | 1020 |
| tatgaaactg gggaccatgt tggagtttac tgtgaaaact tgagtgaagt tgtgaatgat | 1080 |
| gctgaaagat tagtaggatt accaccagac acttactcct ccatccacac tgatagtgaa | 1140 |
| gacgggtcgc cacttggcgg agcctcattg ccgcctcctt tcccgccatg cactttaagg | 1200 |
| aaagcattga cgtgttatgc tgatgttttg agttctccca agaagtcggc tttgcttgca | 1260 |
| ctagctgctc atgccaccga tcccagtgaa gctgatagat tgaaatttct tgcatccccc | 1320 |
| gccggaaagg atgaatattc tcaatggata gttgcaagcc aaagaagtct ccttgaagtc | 1380 |
| atggaagcat tcccgtcagc taagccttca cttggtgttt tctttgcatc tgttgccccg | 1440 |
| cgcttacaac caagatacta ctctatttct tcctcaccca agatggcacc ggataggatt | 1500 |
| catgttacat gtgcattagt ctatgagaaa acacctgcag gccgcatcca caaaggagtt | 1560 |
| tgttcaactt ggatgaagaa cgcagtgcct atgaccgaga gtcaagattg cagttgggcc | 1620 |
| ccaatatacg tccgaacatc caatttcaga ctaccatctg accctaaggt cccggttatc | 1680 |
| atgattggac ctggcactgg tttggctcct tttagaggtt tccttcaaga gcggttagct | 1740 |
| ttaaaggaag ccggaactga cctcggttta tccattttat tcttcggatg taggaatcgc | 1800 |
| aaagtggatt tcatatatga aaacgagctt aacaactttg tggagactgg tgctctttct | 1860 |
| gagcttattg ttgctttctc ccgtgaaggc ccgactaagg aatatgtgca acacaagatg | 1920 |
| agtgagaagg cttcggatat ctggaacttg ctttctgaag gagcatattt atacgtatgt | 1980 |
| ggtgatgcca aaggcatggc caaagatgta catcgaaccc tccacacaat tgtgcaagaa | 2040 |
| cagggatctc ttgactcgtc aaaggcagaa ctctacgtga agaatctaca aatgtcagga | 2100 |
| agatacctcc gtgacgtttg gtaa | 2124 |

SEQ ID NO: 86
Stevia rebaudiana

| | |
|---|---|
| MQSNSVKISP LDLVTALFSG KVLDTSNASE SGESAMLPTI AMIMENRELL MILTTSVAVL | 60 |
| IGCVVVLVWR RSSTKKSALE PPVIVVPKRV QEEEVDDGKK KVTVFFGTQT GTAEGFAKAL | 120 |
| VEEAKARYEK AVFKVIDLDD YAADDDEYEE KLKKESLAFF FLATYGDGEP TDNAARFYKW | 180 |
| FTEGDAKGEW LNKLQYGVFG LGNRQYEHFN KIAKVVDDGL VEQGAKRLVP VGLGDDDQCI | 240 |
| EDDFTAWKEL VWPELDQLLR DEDDTTVATP YTAAVAEYRV VFHEKPDALS EDYSYTNGHA | 300 |
| VHDAQHPCRS NVAVKKELHS PESDRSCTHL EFDISNTGLS YETGDHVGVY CENLSEVVND | 360 |
| AERLVGLPPD TYSSIHTDSE DGSPLGGASL PPPFPPCTLR KALTCYADVL SSPKKSALLA | 420 |
| LAAHATDPSE ADRLKFLASP AGKDEYSQWI VASQRSLLEV MEAFPSAKPS LGVFFASVAP | 480 |
| RLQPRYYSIS SSPKMAPDRI HVTCALVYEK TPAGRIHKGV CSTWMKNAVP MTESQDCSWA | 540 |
| PIYVRTSNFR LPSDPKVPVI MIGPGTGLAP FRGFLQERLA LKEAGTDLGL SILFFGCRNR | 600 |
| KVDFIYENEL NNFVETGALS ELIVAFSREG PTKEYVQHKM SEKASDIWNL LSEGAYLYVC | 660 |
| GDAKGMAKDV HRTLHTIVQE QGSLDSSKAE LYVKNLQMSG RYLRDVW | 707 |

SEQ ID NO: 87
Artificial Sequence

| | |
|---|---|
| atgtcctcca actccgattt ggtcagaaga ttggaatctg ttttgggtgt ttctttcggt | 60 |
| ggttctgtta ctgattccgt tgttgttatt gctaccacct ctattgcttt ggttatcggt | 120 |
| gttttggttt tgttgtggag aagatcctct gacagatcta gagaagttaa gcaattggct | 180 |
| gttccaaagc cagttactat cgttgaagaa gaagatgaat tcgaagttgc ttctggtaag | 240 |
| accagagttt ctattttcta cggtactcaa actggtactg ctgaaggttt tgctaaggct | 300 |
| ttggctgaag aaatcaaagc cagatacgaa aaagctgccg ttaaggttat tgatttggat | 360 |
| gattacacag ccgaagatga caaatacggt gaaaagttga agaaagaaac tatggccttc | 420 |
| ttcatgttgg ctacttatgg tgatggtgaa cctactgata atgctgctag attttacaag | 480 |
| tggttcaccg aaggtactga tagaggtgtt tggttggaac atttgagata cggtgtattc | 540 |
| ggtttgggta acagacaata cgaacacttc aacaagattg ccaaggttgt tgatgatttg | 600 |
| ttggttgaac aaggtgccaa gagattggtt actgttggtt tgggtgatga tgatcaatgc | 660 |
| atcgaagatg atttctccgc ttggaaagaa gccttgtggc cagaattgga tcaattattg | 720 |
| caagatgata ccaacaccgt ttctactcca tacactgctg ttattccaga atacagagtt | 780 |
| gttatccacg atccatctgt tacctcttat gaagatccat actctaacat ggctaacggt | 840 |
| aatgcctctt acgatattca tcatccatgt agagctaacg ttgccgtcca aaaagaattg | 900 |
| cataagccag aatctgacag aagttgcatc catttggaat tcgatatttt cgctactggt | 960 |
| ttgacttacg aaaccggtga tcatgttggt gtttacgctg ataattgtga tgatactgta | 1020 |
| gaagaagccg ctaagttgtt gggtcaacca ttggatttgt tgttctccat tcataccgat | 1080 |
| aacaacgacg gtacttcttt gggttcttct ttgccaccac catttccagg tccatgtact | 1140 |
| ttgagaactg ctttggctag atatgccgat ttgttgaatc caccaaaaaa ggctgctttg | 1200 |
| attgctttag ctgctcatgc tgatgaacca tctgaagctg aaagattgaa gttcttgtca | 1260 |
| tctccacaag gtaaggacga atattctaaa tgggttgtcg gttcccaaag atccttggtt | 1320 |
| gaagttatgg ctgaatttcc atctgctaaa ccaccattgg gtgtattttt tgctgctgtt | 1380 |
| gttcctagat tgcaacctag atattactcc atctcttcca gtccaagatt tgctccacat | 1440 |
| agagttcatg ttacttgcgc tttggtttat ggtccaactc caactggtag aattcacaga | 1500 |
| ggtgtatgtt cattctggat gaagaatgtt gtcccattgg aaaagtctca aaactgttct | 1560 |
| tgggccccaa ttttcatcag acaatctaat ttcaagttgc cagccgatca ttctgttcca | 1620 |
| atagttatgg ttggtccagg tactggttta gctcctttta gaggtttctt acaagaaaga | 1680 |
| ttggccttga aagaagaagg tgctcaagtt ggtcctgctt tgttgttttt tggttgcaga | 1740 |
| aacagacaaa tggacttcat ctacgaagtc gaattgaaca actttgtcga acaaggtgct | 1800 |
| ttgtccgaat tgatcgttgc tttttcaaga gaaggtccat ccaaagaata cgtccaacat | 1860 |
| aagatggttg aaaaggcagc ttacatgtgg aacttgattt ctcaaggtgg ttacttctac | 1920 |
| gtttgtggtg atgctaaagg tatggctaga gatgttcata gaacattgca taccatcgtc | 1980 |
| caacaagaag aaaaggttga ttctaccaag gccgaatcca tcgttaagaa attgcaaatg | 2040 |
| gacggtagat acttgagaga tgtttggtga | 2070 |

SEQ ID NO: 88
Rubus suavissimus

| | |
|---|---|
| MSSNSDLVRR LESVLGVSFG GSVTDSVVVI ATTSIALVIG VLVLLWRRSS DRSREVKQLA | 60 |
| VPKPVTIVEE EDEFEVASGK TRVSIFYGTQ TGTAEGFAKA LAEEIKARYE KAAVKVIDLD | 120 |
| DYTAEDDKYG EKLKKETMAF FMLATYGDGE PTDNAARFYK WFTEGTDRGV WLEHLRYGVF | 180 |
| GLGNRQYEHF NKIAKVVDDL LVEQGAKRLV TVGLGDDDQC IEDDFSAWKE ALWPELDQLL | 240 |
| QDDTNTVSTP YTAVIPEYRV VIHDPSVTSY EDPYSNMANG NASYDIHHPC RANVAVQKEL | 300 |
| HKPESDRSCI HLEFDIFATG LTYETGDHVG VYADNCDDTV EEAAKLLGQP LDLLFSIHTD | 360 |
| NNDGTSLGSS LPPPFPGPCT LRTALARYAD LLNPPKKAAL IALAAHADEP SEAERLKFLS | 420 |
| SPQGKDEYSK WVVGSQRSLV EVMAEFPSAK PPLGVFFAAV VPRLQPRYYS ISSSPRFAPH | 480 |
| RVHVTCALVY GPTPTGRIHR GVCSFWMKNV VPLEKSQNCS WAPIFIRQSN FKLPADHSVP | 540 |
| IVMVGPGTGL APFRGFLQER LALKEEGAQV GPALLFFGCR NRQMDFIYEV ELNNFVEQGA | 600 |
| LSELIVAFSR EGPSKEYVQH KMVEKAAYMW NLISQGGYFY VCGDAKGMAR DVHRTLHTIV | 660 |
| QQEEKVDSTK AESIVKKLQM DGRYLRDVW | 689 |

SEQ ID NO: 89
Artificial Sequence

| | |
|---|---|
| atgacttctg cactttatgc ctccgatctt ttcaaacaat tgaaaagtat catgggaacg | 60 |
| gattctttgt ccgatgatgt tgtattagtt attgctacaa cttctctggc actggttgct | 120 |
| ggtttcgttg tcttattgtg gaaaaagacc acggcagatc gttccggcga gctaaagcca | 180 |
| ctaatgatcc ctaagtctct gatggcgaaa gatgaggatg atgacttaga tctaggttct | 240 |
| ggaaaaacga gagtctctat cttcttcggc acacaaaccg gaacagccga aggattcgct | 300 |
| aaagcacttt cagaagagat caaagcaaga tacgaaaagg cggctgtaaa agtaatcgat | 360 |
| ttggatgatt acgctgccga tgatgaccaa tatgaggaaa agttgaaaaa ggaaacattg | 420 |
| gctttctttt gtgtagccac gtatggtgat ggtgaaccaa ccgataacgc cgcaagattc | 480 |
| tacaagtggt ttactgaaga gaacgaaaga gatatcaagt tgcagcaact tgcttacggc | 540 |
| gtttttgcct taggtaacag acaatacgag cactttaaca agataggtat tgtcttagat | 600 |
| gaagagttat gcaaaaaggg tgcgaagaga ttgattgaag tcggtttagg agatgatgat | 660 |
| caatctatcg aggatgactt taatgcatgg aaggaatctt tgtggtctga attagataag | 720 |
| ttacttaagg acgaagatga taaatccgtt gccactccat acacagccgt cattccagaa | 780 |
| tatagagtag ttactcatga tccaagattc acaacacaga aatcaatgga aagtaatgtg | 840 |
| gctaatggta atactaccat cgatattcat catccatgta gagtagacgt tgcagttcaa | 900 |
| aaggaattgc acactcatga atcagacaga tcttgcatac atcttgaatt tgatatatca | 960 |
| cgtactggta tcacttacga aacaggtgat cacgtgggtg tctacgctga aaaccatgtt | 1020 |
| gaaattgtag aggaagctgg aaagttgttg ggccatagtt tagatcttgt tttctcaatt | 1080 |
| catgccgata aagaggatgg ctcaccacta gaaagtgcag tgcctccacc atttccagga | 1140 |
| ccatgcaccc taggtaccgg tttagctcgt tacgcggatc tgttaaatcc tccacgtaaa | 1200 |
| tcagctctag tggccttggc tgcgtacgcc acagaacctt ctgaggcaga aaaactgaaa | 1260 |
| catctaactt caccagatgg taaggatgaa tactcacaat ggatagtagc tagtcaacgt | 1320 |
| tctttactag aagttatggc tgctttccca tccgctaaac ctcctttggg tgttttcttc | 1380 |
| gccgcaatag cgcctagact gcaaccaaga tactattcaa tttcatcctc acctagactg | 1440 |
| gcaccatcaa gagttcatgt cacatccgct ttagtgtacg gtccaactcc tactggtaga | 1500 |
| atccataagg gcgtttgttc aacatggatg aaaaacgcgg ttccagcaga gaagtctcac | 1560 |
| gaatgttctg gtgctccaat ctttatcaga gcctccaact tcaaactgcc ttccaatcct | 1620 |
| tctactccta ttgtcatggt cggtcctggt acaggtcttg ctccattcag aggtttctta | 1680 |
| caagagagaa tggccttaaa ggaggatggt gaagagttgg gatcttcttt gttgtttttc | 1740 |
| ggctgtagaa acagacaaat ggatttcatc tacgaagatg aactgaataa ctttgtagat | 1800 |
| caaggagtta tttcagagtt gataatggct ttttctagag aaggtgctca gaaggagtac | 1860 |
| gtccaacaca aaatgatgga aaaggccgca caagtttggg acttaatcaa agaggaaggc | 1920 |
| tatctatatg tctgtggtga tgcaaagggt atggcaagag atgttcacag aacacttcat | 1980 |
| actatagtcc aggaacagga aggcgttagt tcttctgaag cggaagcaat tgtgaaaaag | 2040 |
| ttacaaacag agggaagata cttgagagat gtgtggtaa | 2079 |

SEQ ID NO: 90
Arabidopsis thaliana

| | |
|---|---|
| MTSALYASDL FKQLKSIMGT DSLSDDVVLV IATTSLALVA GFVVLLWKKT TADRSGELKP | 60 |
| LMIPKSLMAK DEDDDLDLGS GKTRVSIFFG TQTGTAEGFA KALSEEIKAR YEKAAVKVID | 120 |
| LDDYAADDDQ YEEKLKKETL AFFCVATYGD GEPTDNAARF YKWFTEENER DIKLQQLAYG | 180 |
| VFALGNRQYE HFNKIGIVLD EELCKKGAKR LIEVGLGDDD QSIEDDFNAW KESLWSELDK | 240 |
| LLKDEDDKSV ATPYTAVIPE YRVVTHDPRF TTQKSMESNV ANGNTTIDIH HPCRVDVAVQ | 300 |
| KELHTHESDR SCIHLEFDIS RTGITYETGD HVGVYAENHV EIVEEAGKLL GHSLDLVFSI | 360 |
| HADKEDGSPL ESAVPPPFPG PCTLGTGLAR YADLLNPPRK SALVALAAYA TEPSEAEKLK | 420 |
| HLTSPDGKDE YSQWIVASQR SLLEVMAAFP SAKPPLGVFF AAIAPRLQPR YYSISSSPRL | 480 |
| APSRVHVTSA LVYGPTPTGR IHKGVCSTWM KNAVPAEKSH ECSGAPIFIR ASNFKLPSNP | 540 |
| STPIVMVGPG TGLAPFRGFL QERMALKEDG EELGSSLLFF GCRNRQMDFI YEDELNNFVD | 600 |
| QGVISELIMA FSREGAQKEY VQHKMMEKAA QVWDLIKEEG YLYVCGDAKG MARDVHRTLH | 660 |
| TIVQEQEGVS SSEAEAIVKK LQTEGRYLRD VW | 692 |

SEQ ID NO: 91
Artificial Sequence

| | |
|---|---|
| atgtcttcct cttcctcttc cagtacctct atgattgatt tgatggctgc tattattaaa | 60 |
| ggtgaaccag ttatcgtctc cgacccagca aatgcctctg cttatgaatc agttgctgca | 120 |
| gaattgtctt caatgttgat cgaaaacaga caattcgcca tgatcgtaac tacatcaatc | 180 |
| gctgttttga tcggttgtat tgtcatgttg gtatggagaa gatccggtag tggtaattct | 240 |
| aaaagagtcg aacctttgaa accattagta attaagccaa gagaagaaga aatagatgac | 300 |
| ggtagaaaga aagttacaat atttttcggt acccaaactg gtacagctga aggttttgca | 360 |
| aaagccttag gtgaagaagc taaggcaaga tacgaaaaga ctagattcaa gatagtcgat | 420 |
| ttggatgact atgccgctga tgacgatgaa tacgaagaaa agttgaagaa agaagatgtt | 480 |
| gcatttttct ttttggcaac ctatggtgac ggtgaaccaa ctgacaatgc agccagattc | 540 |
| tacaaatggt ttacagaggg taatgatcgt ggtgaatggt tgaaaaactt aaagtacggt | 600 |
| gttttcggtt tgggtaacag acaatacgaa catttcaaca aagttgcaaa ggttgtcgac | 660 |
| gatattttgg tcgaacaagg tgctcaaaga ttagtccaag taggtttggg tgacgatgac | 720 |
| caatgtatag aagatgactt tactgcctgg agagaagctt tgtggcctga attagacaca | 780 |
| atcttgagag aagaaggtga caccgccgtt gctaccccat atactgctgc agtattagaa | 840 |
| tacagagttt ccatccatga tagtgaagac gcaaagttta atgatatcac tttggccaat | 900 |
| ggtaacggtt atacagtttt cgatgcacaa cacccttaca aagctaacgt tgcagtcaag | 960 |
| agagaattac atacaccaga atccgacaga agttgtatac acttggaatt tgatatcgct | 1020 |
| ggttccggtt taaccatgaa gttgggtgac catgtaggtg ttttatgcga caatttgtct | 1080 |
| gaaactgttg atgaagcatt gagattgttg gatatgtccc ctgacactta ttttagtttg | 1140 |
| cacgctgaaa aagaagatgg tacaccaatt tccagttctt taccacctcc attccctcca | 1200 |
| tgtaacttaa gaacagcctt gaccagatac gcttgcttgt tatcatcccc taaaaagtcc | 1260 |
| gccttggttg ctttagccgc tcatgctagt gatcctactg aagcagaaag attgaaacac | 1320 |
| ttagcatctc cagccggtaa agatgaatat tcaaagtggg tagttgaatc tcaaagatca | 1380 |
| ttgttagaag ttatggcaga atttccatct gccaagcctc cattaggtgt cttctttgct | 1440 |
| ggtgtagcac ctagattgca accaagattc tactcaatca gttcttcacc taagatcgct | 1500 |
| gaaactagaa ttcatgttac atgtgcatta gtctacgaaa agatgccaac cggtagaatt | 1560 |
| cacaagggtg tatgctctac ttggatgaaa aatgctgttc cttacgaaaa atcagaaaag | 1620 |
| ttgttcttag gtagaccaat cttcgtaaga caatcaaact tcaagttgcc ttctgattca | 1680 |
| aaggttccaa taatcatgat aggtcctggt acaggtttag ccccattcag aggtttcttg | 1740 |
| caagaaagat tggctttagt tgaatctggt gtcgaattag gtccttcagt tttgttcttt | 1800 |
| ggttgtagaa acagaagaat ggatttcatc tatgaagaag aattgcaaag attcgtcgaa | 1860 |
| tctggtgcat tggccgaatt atctgtagct ttttcaagag aaggtccaac taaggaatac | 1920 |
| gttcaacata agatgatgga taaggcatcc gacatatgga acatgatcag tcaaggtgct | 1980 |
| tatttgtacg tttgcggtga cgcaaagggt atggccagag atgtccatag atctttgcac | 2040 |
| acaattgctc aagaacaagg ttccatggat agtaccaaag ctgaaggttt cgtaaagaac | 2100 |
| ttacaaactt ccggtagata cttgagagat gtctggtga | 2139 |

SEQ ID NO: 92
Arabidopsis thaliana

| | |
|---|---|
| MSSSSSSSTS MIDLMAAIIK GEPVIVSDPA NASAYESVAA ELSSMLIENR QFAMIVTTSI | 60 |
| AVLIGCIVML VWRRSGSGNS KRVEPLKPLV IKPREEEIDD GRKKVTIFFG TQTGTAEGFA | 120 |
| KALGEEAKAR YEKTRFKIVD LDDYAADDDE YEEKLKKEDV AFFFLATYGD GEPTDNAARF | 180 |
| YKWFTEGNDR GEWLKNLKYG VFGLGNRQYE HFNKVAKVVD DILVEQGAQR LVQVGLGDDD | 240 |
| QCIEDDFTAW REALWPELDT ILREEGDTAV ATPYTAAVLE YRVSIHDSED AKFNDITLAN | 300 |
| GNGYTVFDAQ HPYKANVAVK RELHTPESDR SCIHLEFDIA GSGLTMKLGD HVGVLCDNLS | 360 |
| ETVDEALRLL DMSPDTYFSL HAEKEDGTPI SSSLPPPFPP CNLRTALTRY ACLLSSPKKS | 420 |
| ALVALAAHAS DPTEAERLKH LASPAGKDEY SKWVVESQRS LLEVMAEFPS AKPPLGVFFA | 480 |
| GVAPRLQPRF YSISSSPKIA ETRIHVTCAL VYEKMPTGRI HKGVCSTWMK NAVPYEKSEK | 540 |
| LFLGRPIFVR QSNFKLPSDS KVPIIMIGPG TGLAPFRGFL QERLALVESG VELGPSVLFF | 600 |
| GCRNRRMDFI YEEELQRFVE SGALAELSVA FSREGPTKEY VQHKMMDKAS DIWNMISQGA | 660 |
| YLYVCGDAKG MARDVHRSLH TIAQEQGSMD STKAEGFVKN LQTSGRYLRD VW | 712 |

SEQ ID NO: 93
Artificial Sequence

| | |
|---|---|
| atggaagcct cttacctata catttctatt ttgcttttac tggcatcata cctgttcacc | 60 |
| actcaactta gaaggaagag cgctaatcta ccaccaaccg tgtttccatc aataccaatc | 120 |
| attggacact tatacttact caaaaagcct ctttatagaa ctttagcaaa aattgccgct | 180 |
| aagtacggac caatactgca attacaactc ggctacagac gtgttctggt gatttcctca | 240 |
| ccatcagcag cagaagagtg ctttaccaat aacgatgtaa tcttcgcaaa tagacctaag | 300 |
| acattgtttg gcaaaatagt gggtggaaca tcccttggca gtttatccta cggcgatcaa | 360 |
| tggcgtaatc taaggagagt agcttctatc gaaatcctat cagttcatag gttgaacgaa | 420 |
| tttcatgata tcagagtgga tgagaacaga ttgttaatta gaaaacttag aagttcatct | 480 |
| tctcctgtta ctcttataac agtcttttat gctctaacat tgaacgtcat tatgagaatg | 540 |
| atctctggca aaagatattt cgacagtggg gatagagaat tggaggagga aggtaagaga | 600 |
| tttcgagaaa tcttagacga aacgttgctt ctagccggtg cttctaatgt tggcgactac | 660 |
| ttaccaatat tgaactggtt gggagttaag tctcttgaaa agaaattgat cgctttgcag | 720 |
| aaaaagagag atgacttttt ccagggtttg attgaacagg ttagaaaatc tcgtggtgct | 780 |
| aaagtaggca aaggtagaaa aacgatgatc gaactcttat tatctttgca agagtcagaa | 840 |
| cctgagtact atacagatgc tatgataaga tcttttgtcc taggtctgct ggctgcaggt | 900 |
| agtgatactt cagcgggcac tatggaatgg gccatgagct tactggtcaa tcacccacat | 960 |
| gtattgaaga aagctcaagc tgaaatcgat agagttatcg gtaataacag attgattgac | 1020 |
| gagtcagaca ttggaaatat cccttacatc gggtgtatta tcaatgaaac tctaagactc | 1080 |
| tatccagcag ggccattgtt gttcccacat gaaagttctg ccgactgcgt tatttccggt | 1140 |
| tacaatatac ctagaggtac aatgttaatc gtaaaccaat gggcgattca tcacgatcct | 1200 |
| aaagtctggg atgatcctga aacctttaaa cctgaaagat ttcaaggatt agaaggaact | 1260 |
| agagatggtt tcaaacttat gccattcggt tctgggagaa gaggatgtcc aggtgaaggt | 1320 |
| ttggcaataa ggctgttagg gatgacacta ggctcagtga tccaatgttt tgattgggag | 1380 |
| agagtaggag atgagatggt tgacatgaca gaaggtttgg gtgtcacact tcctaaggcc | 1440 |
| gttccattag ttgccaaatg taagccacgt tccgaaatga ctaatctcct atccgaactt | 1500 |
| taa | 1503 |

SEQ ID NO: 94
S. rebaudiana

| | |
|---|---|
| MEASYLYISI LLLLASYLFT TQLRRKSANL PPTVFPSIPI IGHLYLLKKP LYRTLAKIAA | 60 |
| KYGPILQLQL GYRRVLVISS PSAAEECFTN NDVIFANRPK TLFGKIVGGT SLGSLSYGDQ | 120 |
| WRNLRRVASI EILSVHRLNE FHDIRVDENR LLIRKLRSSS SPVTLITVFY ALTLNVIMRM | 180 |
| ISGKRYFDSG DRELEEEGKR FREILDETLL LAGASNVGDY LPILNWLGVK SLEKKLIALQ | 240 |
| KKRDDFFQGL IEQVRKSRGA KVGKGRKTMI ELLLSLQESE PEYYTDAMIR SFVLGLLAAG | 300 |
| SDTSAGTMEW AMSLLVNHPH VLKKAQAEID RVIGNNRLID ESDIGNIPYI GCIINETLRL | 360 |
| YPAGPLLFPH ESSADCVISG YNIPRGTMLI VNQWAIHHDP KVWDDPETFK PERFQGLEGT | 420 |
| RDGFKLMPFG SGRRGCPGEG LAIRLLGMTL GSVIQCFDWE RVGDEMVDMT EGLGVTLPKA | 480 |
| VPLVAKCKPR SEMTNLLSEL | 500 |

SEQ ID NO: 95
Rubus suavissimus

| | |
|---|---|
| atggaagtaa cagtagctag tagtgtagcc ctgagcctgg tctttattag catagtagta | 60 |
| agatgggcat ggagtgtggt gaattgggtg tggtttaagc cgaagaagct ggaaagattt | 120 |
| ttgagggagc aaggccttaa aggcaattcc tacaggtttt tatatggaga catgaaggag | 180 |
| aactctatcc tgctcaaaca agcaagatcc aaacccatga acctctccac ctcccatgac | 240 |
| atagcacctc aagtcacccc ttttgtcgac caaaccgtga aagcttacgg taagaactct | 300 |
| tttaattggg ttggccccat accaagggtg aacataatga atccagaaga tttgaaggac | 360 |
| gtcttaacaa aaaatgttga ctttgttaag ccaatatcaa acccacttat caagttgcta | 420 |
| gctacaggta ttgcaatcta tgaaggtgag aaatggacta aacacagaag gattatcaac | 480 |
| ccaacattcc attcggagag gctaaagcgt atgttacctt catttcacca aagttgtaat | 540 |
| gagatggtca aggaatggga gagcttggtg tcaaaagagg gttcatcatg tgagttggat | 600 |
| gtctggcctt ttcttgaaaa tatgtcggca gatgtgatct cgagaacagc atttggaact | 660 |
| agctacaaaa aaggacagaa aatctttgaa ctcttgagag agcaagtaat atatgtaacg | 720 |
| aaaggctttc aaagttttta cattccagga tggaggtttc tcccaactaa gatgaacaag | 780 |
| aggatgaatg agattaacga agaaataaaa ggattaatca ggggtattat aattgacaga | 840 |
| gagcaaatca ttaaggcagg tgaagaaacc aacgatgact tattaggtgc acttatggag | 900 |
| tcaaacttga aggacattcg ggaacatggg aaaaacaaca aaaatgttgg gatgagtatt | 960 |
| gaagatgtaa ttcaggagtg taagctgttt tactttgctg ggcaagaaac cacttcagtg | 1020 |
| ttgctggctt ggacaatggt tttacttggt caaaatcaga actggcaaga tcgagcaaga | 1080 |
| caagaggttt tgcaagtctt tggaagcagc aagccagatt ttgatggtct agctcacctt | 1140 |
| aaagtcgtaa ccatgatttt gcttgaagtt cttcgattat acccaccagt cattgaactt | 1200 |
| attcgaacca ttcacaagaa aacacaactt gggaagctct cactaccaga aggagttgaa | 1260 |
| gtccgcttac caacactgct cattcaccat gacaaggaac tgtggggtga tgatgcaaac | 1320 |
| cagttcaatc cagagaggtt ttcggaagga gtttccaaag caacaaagaa ccgactctca | 1380 |
| ttcttcccct tcggagccgg tccacgcatt tgcattggac agaacttttc tatgatggaa | 1440 |
| gcaaagttgg ccttagcatt gatcttgcaa cacttcacct ttgagctttc tccatctcat | 1500 |
| gcacatgctc cttcccatcg tataaccctt caaccacagt atggtgttcg tatcatttta | 1560 |
| catcgacgtt ag | 1572 |

SEQ ID NO: 96
Artificial Sequence

| | |
|---|---|
| atggaagtca ctgtcgcctc ttctgtcgct ttatccttag tcttcatttc cattgtcgtc | 60 |
| agatgggctt ggtccgttgt caactgggtt tggttcaaac caaagaagtt ggaaagattc | 120 |
| ttgagagagc aaggtttgaa gggtaattct tatagattct tgtacggtga catgaaggaa | 180 |
| aattctattt tgttgaagca agccagatcc aaaccaatga acttgtctac ctctcatgat | 240 |
| attgctccac aagttactcc attcgtcgat caaactgtta aagcctacgg taagaactct | 300 |
| ttcaattggg ttggtccaat tcctagagtt aacatcatga acccagaaga tttgaaggat | 360 |
| gtcttgacca agaacgttga cttcgttaag ccaatttcca acccattgat taaattgttg | 420 |
| gctactggta ttgccattta cgaaggtgaa aagtggacta agcatagaag aatcatcaac | 480 |
| cctaccttcc actctgaaag attgaagaga atgttaccat ctttccatca atcctgtaat | 540 |
| gaaatggtta aggaatggga atccttggtt tctaaagaag gttcttcttg cgaattggat | 600 |
| gtttggccat tcttggaaaa tatgtctgct gatgtcattt ccagaaccgc tttcggtacc | 660 |
| tcctacaaga agggtcaaaa gattttcgaa ttgttgagag agcaagttat ttacgttacc | 720 |
| aagggtttcc aatccttcta catcccaggt tggagattct tgccaactaa aatgaacaag | 780 |
| cgtatgaacg agatcaacga agaaattaaa ggtttgatca gaggtattat tatcgacaga | 840 |
| gaacaaatta ttaaagctgg tgaagaaacc aacgatgatt tgttgggtgc tttgatggag | 900 |
| tccaacttga aggatattag agaacatggt aagaacaaca agaatgttgg tatgtctatt | 960 |
| gaagatgtta ttcaagaatg taagttattc tacttcgctg gtcaagagac cacttctgtt | 1020 |
| ttgttagcct ggactatggt cttgttaggt caaaaccaaa attggcaaga tagagctaga | 1080 |
| caagaagttt tgcaagtctt cggttcttcc aagccagact ttgatggttt ggcccacttg | 1140 |
| aaggttgtta ctatgatttt gttagaagtt ttgagattgt acccaccagt cattgagtta | 1200 |
| atcagaacca ttcataaaaa gactcaattg ggtaaattat ctttgccaga aggtgttgaa | 1260 |
| gtcagattac caaccttgtt gattcaccac gataaggaat tatggggtga cgacgctaat | 1320 |
| caatttaatc cagaaagatt ttccgaaggt gtttccaagg ctaccaaaaa ccgtttgtcc | 1380 |
| ttcttcccat ttggtgctgg tccacgtatt tgtatcggtc aaaacttttc catgatggaa | 1440 |
| gccaagttgg ctttggcttt aatcttgcaa cacttcactt tcgaattgtc tccatcccat | 1500 |
| gcccacgctc cttctcatag aatcacttta caaccacaat acggtgtcag aatcatctta | 1560 |
| cacagaagat aa | 1572 |

SEQ ID NO: 97
Rubus suavissimus

| | |
|---|---|
| MEVTVASSVA LSLVFISIVV RWAWSVVNWV WFKPKKLERF LREQGLKGNS YRFLYGDMKE | 60 |
| NSILLKQARS KPMNLSTSHD IAPQVTPFVD QTVKAYGKNS FNWVGPIPRV NIMNPEDLKD | 120 |
| VLTKNVDFVK PISNPLIKLL ATGIAIYEGE KWTKHRRIIN PTFHSERLKR MLPSFHQSCN | 180 |
| EMVKEWESLV SKEGSSCELD VWPFLENMSA DVISRTAFGT SYKKGQKIFE LLREQVIYVT | 240 |
| KGFQSFYIPG WRFLPTKMNK RMNEINEEIK GLIRGIIIDR EQIIKAGEET NDDLLGALME | 300 |
| SNLKDIREHG KNNKNVGMSI EDVIQECKLF YFAGQETTSV LLAWTMVLLG QNQNWQDRAR | 360 |
| QEVLQVFGSS KPDFDGLAHL KVVTMILLEV LRLYPPVIEL IRTIHKKTQL GKLSLPEGVE | 420 |
| VRLPTLLIHH DKELWGDDAN QFNPERFSEG VSKATKNRLS FFPFGAGPRI CIGQNFSMME | 480 |
| AKLALALILQ HFTFELSPSH AHAPSHRITL QPQYGVRIIL HRR | 523 |

SEQ ID NO: 98
Prunus avium

| | |
|---|---|
| atggaagcat caagggctag ttgtgttgcg ctatgtgttg tttgggtgag catagtaatt | 60 |
| acattggcat ggagggtgct gaattgggtg tggttgaggc caaagaaact agaaagatgc | 120 |
| ttgagggagc aaggccttac aggcaattct tacaggcttt tgtttggaga caccaaggat | 180 |
| ctctcgaaga tgctggaaca aacacaatcc aaacccatca aactctccac ctcccatgat | 240 |
| atagcgccac gagtcacccc atttttccat cgaactgtga actctaatgg caagaattct | 300 |
| tttgtttgga tgggccctat accaagagtg cacatcatga atccagaaga tttgaaagat | 360 |
| gccttcaaca gacatgatga ttttcataag acagtaaaaa atcctatcat gaagtctcca | 420 |
| ccaccgggca ttgtaggcat tgaaggtgag caatgggcta aacacagaaa gattatcaac | 480 |
| ccagcattcc atttagagaa gctaaagggt atggtaccaa tattttacca aagttgtagc | 540 |
| gagatgatta acaaatggga gagcttggtg tccaaagaga gttcatgtga gttggatgtg | 600 |
| tggccttatc ttgaaaattt taccagcgat gtgatttccc gagctgcatt tggaagtagc | 660 |
| tatgaagagg gaaggaaaat atttcaacta ctaagagagg aagcaaaagt ttattcggta | 720 |
| gctctacgaa gtgtttacat tccaggatgg aggtttctac caaccaagca gaacaagaag | 780 |
| acgaaggaaa ttcacaatga aattaaaggc ttacttaagg gcattataaa taaaagggaa | 840 |
| gaggcgatga aggcagggga agccactaaa gatgacttac taggaatact tatggagtcc | 900 |
| aacttcaggg aaattcagga acatgggaac aacaaaaatg ctggaatgag tattgaagat | 960 |
| gtaattggag agtgtaagtt gttttacttt gctgggcaag agaccacttc ggtgttgctt | 1020 |
| gtttggacaa tgattttact aagccaaaat caggattggc aagctcgtgc aagagaagag | 1080 |
| gtcttgaaag tctttggaag caacatccca acctatgaag agctaagtca cctaaaagtt | 1140 |
| gtgaccatga ttttacttga agttcttcga ttatacccat cagtcgttgc gcttcctcga | 1200 |
| accactcaca agaaaacaca gcttggaaaa ttatcattac cagctggagt ggaagtctcc | 1260 |
| ttgcccatac tgcttgttca ccatgacaaa gagttgtggg gtgaggatgc aaatgagttc | 1320 |
| aagccagaga ggttttcaga gggagtttca aaggcaacaa agaacaaatt tacatactta | 1380 |
| cctttcggag ggggtccaag gatttgcatt ggacaaaact ttgccatggt ggaagctaaa | 1440 |
| ttggccttgg ccctgatttt acaacacttt gcctttgagc tttctccatc ctatgctcat | 1500 |
| gctccttctg cagttataac ccttcaacct caatttggtg ctcatatcat tttgcataaa | 1560 |
| cgttga | 1566 |

SEQ ID NO: 99
Artificial Sequence

| | |
|---|---|
| atggaagctt ctagagcatc ttgtgttgct ttgtgtgttg tttgggtttc catcgttatt | 60 |
| actttggctt ggagagtttt gaattgggtc tggttaagac caaaaaagtt ggaaagatgc | 120 |
| ttgagagaac aaggtttgac tggtaactct tacagattgt tgttcggtga taccaaggac | 180 |
| ttgtctaaga tgttggaaca aactcaatcc aagcctatca agttgtctac ctctcatgat | 240 |
| attgctccaa gagttactcc attcttccat agaactgtta actccaacgg taagaactct | 300 |
| tttgtttgga tgggtccaat tccaagagtc catattatga accctgaaga tttgaaggac | 360 |
| gctttcaaca gacatgatga tttccataag accgtcaaga acccaattat gaagtctcca | 420 |
| ccaccaggta tagttggtat tgaaggtgaa caatgggcca aacatagaaa gattattaac | 480 |
| ccagccttcc acttggaaaa gttgaaaggt atggttccaa tcttctacca atcctgctct | 540 |
| gaaatgatta acaagtggga atccttggtt tccaaagaat cttcctgtga attggatgtc | 600 |
| tggccatatt tggaaaactt cacctccgat gttatttcca gagctgcttt tggttcttct | 660 |
| tacgaagaag gtagaaagat cttccaatta ttgagagaag aagccaaggt ttactccgtt | 720 |
| gctttgagat ctgtttacat tccaggttgg agattcttgc caactaagca aaacaaaaag | 780 |
| accaaagaaa tccacaacga aatcaagggt ttgttgaagg gtatcatcaa caagagagaa | 840 |
| gaagctatga aggctggtga agctacaaaa gatgatttgt tgggtatctt gatggaatcc | 900 |
| aacttcagag aaatccaaga acacggtaac aacaagaatg ccggtatgtc tattgaagat | 960 |
| gttatcggtg aatgcaagtt gttctacttt gctggtcaag aaactacctc cgttttgttg | 1020 |
| gtttggacca tgattttgtt gtcccaaaat caagattggc aagctagagc tagagaagaa | 1080 |
| gtcttgaaag ttttcggttc taacatccca acctacgaag aattgtctca cttgaaggtt | 1140 |
| gtcactatga tcttgttgga agtattgaga ttatacccat ccgttgttgc attgccaaga | 1200 |
| actactcata agaaaactca attgggtaaa ttgtccttgc cagctggtgt tgaagtttct | 1260 |
| ttgccaattt tgttagtcca ccacgacaaa gaattgtggg gtgaagatgc taatgaattc | 1320 |
| aagccagaaa gattctccga aggtgtttct aaagctacca agaacaagtt cacttacttg | 1380 |
| ccatttggtg gtggtccaag aatatgtatt ggtcaaaatt tcgctatggt cgaagctaaa | 1440 |
| ttggctttgg ctttgatctt gcaacatttc gctttcgaat tgtcaccatc ttatgctcat | 1500 |
| gctccatctg ctgttattac attgcaacca caatttggtg cccatatcat cttgcataag | 1560 |
| agataac | 1567 |

SEQ ID NO: 100
Prunus avium

| | |
|---|---|
| MEASRASCVA LCVVWVSIVI TLAWRVLNWV WLRPKKLERC LREQGLTGNS YRLLFGDTKD | 60 |
| LSKMLEQTQS KPIKLSTSHD IAPRVTPFFH RTVNSNGKNS FVWMGPIPRV HIMNPEDLKD | 120 |
| AFNRHDDFHK TVKNPIMKSP PPGIVGIEGE QWAKHRKIIN PAFHLEKLKG MVPIFYQSCS | 180 |
| EMINKWESLV SKESSCELDV WPYLENFTSD VISRAAFGSS YEEGRKIFQL LREEAKVYSV | 240 |
| ALRSVYIPGW RFLPTKQNKK TKEIHNEIKG LLKGIINKRE EAMKAGEATK DDLLGILMES | 300 |
| NFREIQEHGN NKNAGMSIED VIGECKLFYF AGQETTSVLL VWTMILLSQN QDWQARAREE | 360 |
| VLKVFGSNIP TYEELSHLKV VTMILLEVLR LYPSVVALPR TTHKKTQLGK LSLPAGVEVS | 420 |
| LPILLVHHDK ELWGEDANEF KPERFSEGVS KATKNKFTYL PFGGGPRICI GQNFAMVEAK | 480 |
| LALALILQHF AFELSPSYAH APSAVITLQP QFGAHIILHK R | 521 |

SEQ ID NO: 101
Prunus mume

| | |
|---|---|
| ASWVAVLSVV WVSMVIAWAW RVLNWVWLRP KKLEKCLREQ GLAGNSYRLL FGDTKDLSKM | 60 |
| LEQTQSKPIK LSTSHDIAPH VTPFFHQTVN SYGKNSFVWM GPIPRVHIMN PEDLKDTFNR | 120 |
| HDDFHKVVKN PIMKSLPQGI VGIEGEQWAK HRKIINPAFH LEKLKGMVPI FYRSCSEMIN | 180 |
| KWESLVSKES SCELDVWPYL ENFTSDVISR AAFGSSYEEG RKIFQLLREE AKIYTVAMRS | 240 |
| VYIPGWRFLP TKQNKKAKEI HNEIKGLLKG IINKREEAMK AGEATKDDLL GILMESNFRE | 300 |
| IQEHGNNKNA GMSIEDVIGE CKLFYFAGQE TTSVLLVWTM VLLSQNQDWQ ARAREEVLQV | 360 |
| FGSNIPTYEE LSQLKVVTMI LLEVLRLYPS VVALPRTTHK KTQLGKLSLP AGVEVSLPIL | 420 |
| LVHHDKELWG EDANEFKPER FSEGVSKATK NQFTYFPFGG GPRICIGQNF AMMEAKLALS | 480 |
| LILRHFALEL SPLYAHAPSV TITLQPQYGA HIILHKR | 517 |

SEQ ID NO: 102
Prunus mume

| | |
|---|---|
| MEASRPSCVA LSVVLVSIVI AWAWRVLNWV WLRPNKLERC LREQGLTGNS YRLLFGDTKE | 60 |
| ISMMVEQAQS KPIKLSTTHD IAPRVIPFSH QIVYTYGRNS FVWMGPTPRV TIMNPEDLKD | 120 |
| AFNKSDEFQR AISNPIVKSI SQGLSSLEGE KWAKHRKIIN PAFHLEKLKG MLPTFYQSCS | 180 |
| EMINKWESLV FKEGSREMDV WPYLENLTSD VISRAAFGSS YEEGRKIFQL LREEAKFYTI | 240 |
| AARSVYIPGW RFLPTKQNKR MKEIHKEVRG LLKGIINKRE DAIKAGEAAK GNLLGILMES | 300 |
| NFREIQEHGN NKNAGMSIED VIGECKLFYF AGQETTSVLL VWTLVLLSQN QDWQARAREE | 360 |
| VLQVFGTNIP TYDQLSHLKV VTMILLEVLR LYPAVVELPR TTYKKTQLGK FLLPAGVEVS | 420 |
| LHIMLAHHDK ELWGEDAKEF KPERFSEGVS KATKNQFTYF PFGAGPRICI GQNFAMLEAK | 480 |
| LALSLILQHF TFELSPSYAH APSVTITLHP QFGAHFILHK R | 521 |

SEQ ID NO: 103
Prunus mume

| | |
|---|---|
| CVALSVVLVS IVIAWAWRVL NWVWLRPNKL ERCLREQGLT GNSYRLLFGD TKEISMMVEQ | 60 |
| AQSKPIKLST THDIAPRVIP FSHQIVYTYG RNSFVWMGPT PRVTIMNPED LKDAFNKSDE | 120 |
| FQRAISNPIV KSISQGLSSL EGEKWAKHRK IINPAFHLEK LKGMLPTFYQ SCSEMINKWE | 180 |
| SLVFKEGSRE MDVWPYLENL TSDVISRAAF GSSYEEGRKI FQLLREEAKF YTIAARSVYI | 240 |
| PGWRFLPTKQ NKRMKEIHKE VRGLLKGIIN KREDAIKAGE AAKGNLLGIL MESNFREIQE | 300 |
| HGNNKNAGMS IEDVIGECKL FYFAGQETTS VLLVWTLVLL SQNQDWQARA REEVLQVFGT | 360 |
| NIPTYDQLSH LKVVTMILLE VLRLYPAVVE LPRTTYKKTQ LGKFLLPAGV EVSLHIMLAH | 420 |
| HDKELWGEDA KEFKPERFSE GVSKATKNQF TYFPFGAGPR ICIGQNFAML EAKLALSLIL | 480 |
| QHFTFELSPS YAHAPSVTIT LHPQFGAHFI LHKR | 514 |

SEQ ID NO: 104
Prunus persica

| | |
|---|---|
| MGPIPRVHIM NPEDLKDTFN RHDDFHKVVK NPIMKSLPQG IVGIEGDQWA KHRKIINPAF | 60 |
| HLEKLKGMVP IFYQSCSEMI NIWKSLVSKE SSCELDVWPY LENFTSDVIS RAAFGSSYEE | 120 |
| GRKIFQLLRE EAKVYTVAVR SVYIPGWRFL PTKQNKKTKE IHNEIKGLLK GIINKREEAM | 180 |
| KAGEATKDDL LGILMESNFR EIQEHGNNKN AGMSIEDVIG ECKLFYFAGQ ETTSVLLVWT | 240 |
| MVLLSQNQDW QARAREEVLQ VFGSNIPTYE ELSHLKVVTM ILLEVLRLYP SVVALPRTTH | 300 |
| KKTQLGKLSL PAGVEVSLPI LLVHHDKELW GEDANEFKPE RFSEGVSKAT KNQFTYFPFG | 360 |
| GGPRICIGQN FAMMEAKLAL SLILQHFTFE LSPQYSHAPS VTITLQPQYG AHLILHKR | 418 |

SEQ ID NO: 105
Artificial Sequence

| | |
|---|---|
| atgggtttgt tcccattaga ggattcctac gcgctggtct ttgaaggact agcaataaca | 60 |
| ctggctttgt actatctact gtctttcatc tacaaaacat ctaaaaagac atgtacacct | 120 |
| cctaaagcat ctggtgaaat cattccaatt acaggaatca tattgaatct gctatctggc | 180 |
| tcaagtggtc tacctattat cttagcactt gcctctttag cagacagatg tggtcctatt | 240 |
| ttcaccatta ggctgggtat taggagagtg ctagtagtat caaattggga aatcgctaag | 300 |
| gagattttca ctacccacga tttgatagtt tctaatagac caaaatactt agccgctaag | 360 |
| attcttggtt tcaattatgt ttcattctct ttcgctccat acggcccata ttgggtcgga | 420 |
| atcagaaaga ttattgctac aaaactaatg tcttcttcca gacttcagaa gttgcaattt | 480 |
| gtaagagttt ttgaactaga aaactctatg aaatctatca gagaatcatg gaaggagaaa | 540 |
| aaggatgaag agggaaaggt attagttgag atgaaaaagt ggttctggga actgaatatg | 600 |
| aacatagtgt taaggacagt tgctggtaaa caatacactg gtacagttga tgatgccgat | 660 |
| gcaaagcgta tctccgagtt attcagagaa tggtttcact acactggcag atttgtcgtt | 720 |
| ggagacgctt ttccttttct aggttggttg gacctgggcg gatacaaaaa gacaatggaa | 780 |
| ttagttgcta gtagattgga ctcaatggtc agtaaatggt tagatgagca tcgtaaaaag | 840 |
| caagctaacg atgacaaaaa ggaggatatg gatttcatgg atatcatgat ctccatgaca | 900 |
| gaagcaaatt caccacttga aggatacggc actgatacta ttatcaagac cacatgtatg | 960 |
| actttgattg tttcaggagt tgatacaacc tcaatcgtac ttacttgggc cttatcactt | 1020 |
| ttgttaaaca acagagatac tttgaaaaag gcacaagagg aattagatat gtgcgtaggt | 1080 |
| aaaggaagac aagtcaacga gtctgatctt gttaacttga tatacttgga agcagtgctt | 1140 |
| aaagaggctt taagacttta cccagcagcg ttcttaggcg gaccaagagc attcttggaa | 1200 |
| gattgtactg ttgctggtta tagaattcca aagggcacct gcttgttgat taacatgtgg | 1260 |
| aaactgcata gagatccaaa catttggagt gatccttgcg aattcaagcc agaaagattt | 1320 |
| ttgacaccta atcaaaagga tgttgatgtg atcggtatgg atttcgaatt gataccattt | 1380 |
| ggtgccggca gaagatattg tccaggtact agattggctt tacagatgtt gcatatcgta | 1440 |
| ttagcgacat tgctgcaaaa cttcgaaatg tcaacaccaa acgatgcgcc agtcgatatg | 1500 |
| actgcttctg ttggcatgac aaatgccaaa gcatcacctt tagaagtctt gctatcacct | 1560 |
| cgtgttaaat ggtcctaa | 1578 |

SEQ ID NO: 106
Stevia rebaudiana

| | |
|---|---|
| MGLFPLEDSY ALVFEGLAIT LALYYLLSFI YKTSKKTCTP PKASGEHPIT GHLNLLSGSS | 60 |
| GLPHLALASL ADRCGPIFTI RLGIRRVLVV SNWEIAKEIF TTHDLIVSNR PKYLAAKILG | 120 |
| FNYVSFSFAP YGPYWVGIRK IIATKLMSSS RLQKLQFVRV FELENSMKSI RESWKEKKDE | 180 |
| EGKVLVEMKK WFWELNMNIV LRTVAGKQYT GTVDDADAKR ISELFREWFH YTGRFVVGDA | 240 |
| FPFLGWLDLG GYKKTMELVA SRLDSMVSKW LDEHRKKQAN DDKKEDMDFM DIMISMTEAN | 300 |
| SPLEGYGTDT IIKTTCMTLI VSGVDTTSIV LTWALSLLLN NRDTLKKAQE ELDMCVGKGR | 360 |
| QVNESDLVNL IYLEAVLKEA LRLYPAAFLG GPRAFLEDCT VAGYRIPKGT CLLINMWKLH | 420 |
| RDPNIWSDPC EFKPERFLTP NQKDVDVIGM DFELIPFGAG RRYCPGTRLA LQMLHIVLAT | 480 |
| LLQNFEMSTP NDAPVDMTAS VGMTNAKASP LEVLLSPRVK WS | 522 |

SEQ ID NO: 107
Artificial Sequence

| | |
|---|---|
| atgatacaag ttttaactcc aattctactc ttcctcatct tcttcgtttt ctggaaagtc | 60 |
| tacaaacatc aaaagactaa aatcaatcta ccaccaggtt ccttcggctg gccatttttg | 120 |
| ggtgaaacct tagccttact tagagcaggc tgggattctg agccagaaag attcgtaaga | 180 |
| gagcgtatca aaaagcatgg atctccactt gttttcaaga catcactatt tggagacaga | 240 |
| ttcgctgttc tttgcggtcc agctggtaat aagtttttgt tctgcaacga aaacaaatta | 300 |
| gtggcatctt ggtggccagt ccctgtaagg aagttgttcg gtaaaagttt actcacaata | 360 |
| agaggagatg aagcaaaatg gatgagaaaa atgctattgt cttacttggg tccagatgca | 420 |
| tttgccacac attatgccgt tactatggat gttgtaacac gtagacatat tgatgtccat | 480 |
| tggaggggca aggaggaagt taatgtattt caaacagtta agttgtacgc attcgaatta | 540 |
| gcttgtagat tattcatgaa cctagatgac ccaaaccaca tcgcgaaact cggtagtctt | 600 |
| ttcaacattt tcctcaaagg gatcatcgag cttcctatag acgttcctgg aactagattt | 660 |
| tactccagta aaaaggccgc agctgccatt agaattgaat tgaaaaagct cattaaagct | 720 |
| agaaaactcg aattgaagga gggtaaggcg tcttcttcac aggacttgct ttctcatcta | 780 |
| ttaacatcac ctgatgagaa tgggatgttc ttgacagaag aggaaatagt cgataacatt | 840 |
| ctacttttgt tattcgctgg tcacgatacc tctgcactat caataacact tttgatgaaa | 900 |
| accttaggtg aacacagtga tgtgtacgac aaggttttga aggaacaatt agaaatttcc | 960 |
| aaaacaaagg aggcttggga atcactaaag tgggaagata tccagaagat gaagtactca | 1020 |
| tggtcagtaa tctgtgaagt catgagattg aatcctcctg tcatagggac atacagagag | 1080 |
| gcgttggttg atatcgacta tgctggttac actatcccaa aaggatggaa gttgcattgg | 1140 |
| tcagctgttt ctactcaaag agacgaagcc aatttcgaag atgtaactag attcgatcca | 1200 |
| tccagatttg aaggggcagg ccctactcca ttcacatttg tgcctttcgg tggaggtcct | 1260 |
| agaatgtgtt taggcaaaga gtttgccagg ttagaagtgt tagcatttct ccacaacatt | 1320 |
| gttaccaact ttaagtggga tcttctaatc cctgatgaga agatcgaata tgatccaatg | 1380 |
| gctactccag ctaagggctt gccaattaga cttcatccac accaagtcta a | 1431 |

SEQ ID NO: 108
Stevia rebaudiana

| | |
|---|---|
| MIQVLTPILL FLIFFVFWKV YKHQKTKINL PPGSFGWPFL GETLALLRAG WDSEPERFVR | 60 |
| ERIKKHGSPL VFKTSLFGDR FAVLCGPAGN KFLFCNENKL VASWWPVPVR KLFGKSLLTI | 120 |
| RGDEAKWMRK MLLSYLGPDA FATHYAVTMD VVTRRHIDVH WRGKEEVNVF QTVKLYAFEL | 180 |
| ACRLFMNLDD PNHIAKLGSL FNIFLKGIIE LPIDVPGTRF YSSKKAAAAI RIELKKLIKA | 240 |
| RKLELKEGKA SSSQDLLSHL LTSPDENGMF LTEEEIVDNI LLLLFAGHDT SALSITLLMK | 300 |
| TLGEHSDVYD KVLKEQLEIS KTKEAWESLK WEDIQKMKYS WSVICEVMRL NPPVIGTYRE | 360 |
| ALVDIDYAGY TIPKGWKLHW SAVSTQRDEA NFEDVTRFDP SRFEGAGPTP FTFVPFGGGP | 420 |
| RMCLGKEFAR LEVLAFLHNI VTNFKWDLLI PDEKIEYDPM ATPAKGLPIR LHPHQV | 476 |

SEQ ID NO: 109
Artificial Sequence

| | |
|---|---|
| atggagtctt tagtggttca tacagtaaat gctatctggt gtattgtaat cgtcgggatt | 60 |
| ttctcagttg gttatcacgt ttacggtaga gctgtggtcg aacaatggag aatgagaaga | 120 |
| tcactgaagc tacaaggtgt taaaggccca ccaccatcca tcttcaatgg taacgtctca | 180 |
| gaaatgcaac gtatccaatc cgaagctaaa cactgctctg gcgataacat tatctcacat | 240 |
| gattattctt cttcattatt cccacacttc gatcactgga gaaaacagta cggcagaatc | 300 |
| tacacatact ctactggatt aaagcaacac ttgtacatca atcatccaga aatggtgaag | 360 |
| gagctatctc agactaacac attgaacttg ggtagaatca cccatataac caaaagattg | 420 |
| aatcctatct taggtaacgg aatcataacc tctaatggtc ctcattgggc ccatcagcgt | 480 |
| agaattatcg cctacgagtt tactcatgat aagatcaagg gtatggttgg tttgatggtt | 540 |
| gagtctgcta tgcctatgtt gaataagtgg gaggagatgg taaagagagg cggagaaatg | 600 |
| ggatgcgaca taagagttga tgaggacttg aaagatgttt cagcagatgt gattgcaaaa | 660 |
| gcctgtttcg gatcctcatt ttctaaaggt aaggctattt tctctatgat aagagatttg | 720 |
| cttacagcta tcacaaagag aagtgttcta ttcagattca acggattcac tgatatggtc | 780 |
| tttgggagta aaaagcatgg tgacgttgat atagacgctt tagaaatgga attggaatca | 840 |
| tccatttggg aaactgtcaa ggaacgtgaa atagaatgta aagatactca caaaaaggat | 900 |
| ctgatgcaat tgattttgga aggggcaatg cgttcatgtg acggtaacct ttgggataaa | 960 |
| tcagcatata gaagatttgt tgtagataat tgtaaatcta tctacttcgc agggcatgat | 1020 |
| agtacagctg tctcagtgtc atggtgtttg atgttactgg ccctaaaccc atcatggcaa | 1080 |
| gttaagatcc gtgatgaaat tctgtcttct tgcaaaaatg gtattccaga tgccgaaagt | 1140 |
| atcccaaacc ttaaaacagt gactatggtt attcaagaga caatgagatt ataccctcca | 1200 |
| gcaccaatcg tcgggagaga agcctctaaa gatatcagat tgggcgatct agttgttcct | 1260 |
| aaaggcgtct gtatatggac actaatacca gctttacaca gagatcctga gatttgggga | 1320 |
| ccagatgcaa acgatttcaa accagaaaga ttttctgaag gaatttcaaa ggcttgtaag | 1380 |
| tatcctcaaa gttacattcc atttggtctg ggtcctagaa catgcgttgg taaaaacttt | 1440 |
| ggcatgatgg aagtaaaggt tcttgtttcc ctgattgtct ccaagttctc tttcactcta | 1500 |
| tctcctacct accaacatag tcctagtcac aaacttttag tagaaccaca acatggggtg | 1560 |
| gtaattagag tggtttaa | 1578 |

SEQ ID NO: 110
Arabidopsis thaliana

| | |
|---|---|
| MESLVVHTVN AIWCIVIVGI FSVGYHVYGR AVVEQWRMRR SLKLQGVKGP PPSIFNGNVS | 60 |
| EMQRIQSEAK HCSGDNIISH DYSSSLFPHF DHWRKQYGRI YTYSTGLKQH LYINHPEMVK | 120 |
| ELSQTNTLNL GRITHITKRL NPILGNGIIT SNGPHWAHQR RIIAYEFTHD KIKGMVGLMV | 180 |
| ESAMPMLNKW EEMVKRGGEM GCDIRVDEDL KDVSADVIAK ACFGSSFSKG KAIFSMIRDL | 240 |
| LTAITKRSVL FRFNGFTDMV FGSKKHGDVD IDALEMELES SIWETVKERE IECKDTHKKD | 300 |
| LMQLILEGAM RSCDGNLWDK SAYRRFVVDN CKSIYFAGHD STAVSVSWCL MLLALNPSWQ | 360 |
| VKIRDEILSS CKNGIPDAES IPNLKTVTMV IQETMRLYPP APIVGREASK DIRLGDLVVP | 420 |
| KGVCIWTLIP ALHRDPEIWG PDANDFKPER FSEGISKACK YPQSYIPFGL GPRTCVGKNF | 480 |
| GMMEVKVLVS LIVSKFSFTL SPTYQHSPSH KLLVEPQHGV VIRVV | 525 |

SEQ ID NO: 111
Artificial Sequence

| | |
|---|---|
| atgtacttcc tactacaata cctcaacatc acaaccgttg gtgtctttgc cacattgttt | 60 |
| ctctcttatt gtttacttct ctggagaagt agagcgggta acaaaaagat tgccccagaa | 120 |
| gctgccgctg catggcctat tatcggccac ctccacttac ttgcaggtgg atcccatcaa | 180 |
| ctaccacata ttacattggg taacatggca gataagtacg gtcctgtatt cacaatcaga | 240 |
| ataggcttgc atagagctgt agttgtctca tcttgggaaa tggcaaagga atgttcaaca | 300 |
| gctaatgatc aagtgtcttc ttcaagacct gaactattag cttctaagtt gttgggttat | 360 |
| aactacgcca tgtttggttt ttcaccatac ggttcatact ggagagaaat gagaaagatc | 420 |
| atctctctcg aattactatc taattccaga ttggaactat tgaaagatgt tagagcctca | 480 |
| gaagttgtca catctattaa ggaactatac aaattgtggg cggaaaagaa gaatgagtca | 540 |
| ggattggttt ctgtcgagat gaaacaatgg ttcggagatt tgactttaaa cgtgatcttg | 600 |
| agaatggtgg ctggtaaaag atacttctcc gcgagtgacg cttcagaaaa caaacaggcc | 660 |
| cagcgttgta gaagagtctt cagagaattc ttccatctct ccggcttgtt tgtggttgct | 720 |
| gatgctatac cttttcttgg atggctcgat tggggaagac acgagaagac cttgaaaaag | 780 |
| accgccatag aaatggattc catcgcccag gagtggcttg aggaacatag acgtagaaaa | 840 |
| gattctggag atgataattc tacccaagat ttcatggacg ttatgcaatc tgtgctagat | 900 |
| ggcaaaaatc taggcggata cgatgctgat acgattaaca aggctacatg cttaactctt | 960 |
| atatcaggtg gcagtgatac tactgtagtt tctttgacat gggctcttag tcttgtgtta | 1020 |
| aacaatagag atactttgaa aaaggcacag gaagagttag acatccaagt cggtaaggaa | 1080 |
| agattggtta acgagcaaga catcagtaag ttagtttact tgcaagcaat agtaaaagag | 1140 |
| acactcagac tttatccacc aggtcctttg ggtggtttga gacaattcac tgaagattgt | 1200 |
| acactaggtg gctatcacgt ttcaaaagga actagattaa tcatgaactt atccaagatt | 1260 |
| caaaaagatc cacgtatttg gtctgatcct actgaattcc aaccagagag attccttacg | 1320 |
| actcataaag atgtcgatcc acgtggtaaa cactttgaat tcattccatt cggtgcagga | 1380 |
| agacgtgcat gtcctggtat cacattcgga ttacaagtac tacatctaac attggcatct | 1440 |
| ttcttgcatg cgtttgaatt ttcaacacca tcaaatgagc aggttaacat gagagaatca | 1500 |
| ttaggtctta cgaatatgaa atctacccca ttagaagttt tgatttctcc aagactatcc | 1560 |
| cttaattgct tcaaccttat gaaaatttga | 1590 |

SEQ ID NO: 112
Vitis vinifera

| | |
|---|---|
| MYFLLQYLNI TTVGVFATLF LSYCLLLWRS RAGNKKIAPE AAAAWPIIGH LHLLAGGSHQ | 60 |
| LPHITLGNMA DKYGPVFTIR IGLHRAVVVS SWEMAKECST ANDQVSSSRP ELLASKLLGY | 120 |
| NYAMFGFSPY GSYWREMRKI ISLELLSNSR LELLKDVRAS EVVTSIKELY KLWAEKKNES | 180 |
| GLVSVEMKQW FGDLTLNVIL RMVAGKRYFS ASDASENKQA QRCRRVFREF FHLSGLFVVA | 240 |
| DAIPFLGWLD WGRHEKTLKK TAIEMDSIAQ EWLEEHRRRK DSGDDNSTQD FMDVMQSVLD | 300 |
| GKNLGGYDAD TINKATCLTL ISGGSDTTVV SLTWALSLVL NNRDTLKKAQ EELDIQVGKE | 360 |
| RLVNEQDISK LVYLQAIVKE TLRLYPPGPL GGLRQFTEDC TLGGYHVSKG TRLIMNLSKI | 420 |
| QKDPRIWSDP TEFQPERFLT THKDVDPRGK HFEFIPFGAG RRACPGITFG LQVLHLTLAS | 480 |
| FLHAFEFSTP SNEQVNMRES LGLTNMKSTP LEVLISPRLS SCSLYN | 526 |

SEQ ID NO: 113
Artificial Sequence

| | |
|---|---|
| atggaaccta acttttactt gtcattacta ttgttgttcg tgaccttcat ttctttaagt | 60 |
| ctgtttttca tcttttacaa acaaaagtcc ccattgaatt tgccaccagg gaaaatgggt | 120 |
| taccctatca taggtgaaag tttagaattc ctatccacag gctggaaggg acatcctgaa | 180 |
| aagttcatat ttgatagaat gcgtaagtac agtagtgagt tattcaagac ttctattgta | 240 |
| ggcgaatcca cagttgtttg ctgtggggca gctagtaaca aattcctatt ctctaacgaa | 300 |
| aacaaactgg taactgcctg gtggccagat tctgttaaca aaatcttccc aacaacttca | 360 |
| ctggattcta atttgaagga ggaatctata aagatgagaa agttgctgcc acagttcttc | 420 |
| aaaccagaag cacttcaaag atacgtcggc gttatggatg taatcgcaca aagacatttt | 480 |
| gtcactcact gggacaacaa aaatgagatc acagtttatc cacttgctaa aagatacact | 540 |
| ttcttgcttg cgtgtagact gttcatgtct gttgaggatg aaaatcatgt ggcgaaattc | 600 |
| tcagacccat tccaactaat cgctgcaggc atcatttcac ttcctatcga tcttcctggt | 660 |
| actccattca acaaggccat aaaggcttca aatttcatta gaaaagagct gataaagatt | 720 |
| atcaaacaaa gacgtgttga tctggcagag ggtacagcat ctccaaccca ggatatcttg | 780 |
| tcacatatgc tattaacatc tgatgaaaac ggtaaatcta tgaacgagtt gaacattgcc | 840 |
| gacaagattc ttggactatt gataggaggc cacgatacag cttcagtagc ttgcacattt | 900 |
| ctagtgaagt acttaggaga attaccacat atctacgata aagtctacca agagcaaatg | 960 |
| gaaattgcca agtccaaacc tgctggggaa ttgttgaatt gggatgactt gaaaaagatg | 1020 |
| aagtattcat ggaatgtggc atgtgaggta atgagattgt caccaccttt acaaggtggt | 1080 |
| tttagagagg ctataactga ctttatgttt aacggtttct ctattccaaa agggtggaag | 1140 |
| ttatactggt ccgccaactc tacacacaaa aatgcagaat gtttcccaat gcctgagaaa | 1200 |
| ttcgatccta ccagatttga aggtaatggt ccagcgcctt atacatttgt accattcggt | 1260 |
| ggaggcccta gaatgtgtcc tggaaaggaa tacgctagat tagaaatctt ggttttcatg | 1320 |
| cataatctgg tcaaacgttt taagtgggaa aaggttattc cagacgaaaa gattattgtc | 1380 |
| gatccattcc caatcccagc taaagatctt ccaatccgtt tgtatcctca caaagcttaa | 1440 |

SEQ ID NO: 114
Medicago truncatula

| | |
|---|---|
| MEPNFYLSLL LLFVTFISLS LFFIFYKQKS PLNLPPGKMG YPIIGESLEF LSTGWKGHPE | 60 |
| KFIFDRMRKY SSELFKTSIV GESTVVCCGA ASNKFLFSNE NKLVTAWWPD SVNKIFPTTS | 120 |
| LDSNLKEESI KMRKLLPQFF KPEALQRYVG VMDVIAQRHF VTHWDNKNEI TVYPLAKRYT | 180 |
| FLLACRLFMS VEDENHVAKF SDPFQLIAAG IISLPIDLPG TPFNKAIKAS NFIRKELIKI | 240 |
| IKQRRVDLAE GTASPTQDIL SHMLLTSDEN GKSMNELNIA DKILGLLIGG HDTASVACTF | 300 |
| LVKYLGELPH IYDKVYQEQM EIAKSKPAGE LLNWDDLKKM KYSWNVACEV MRLSPPLQGG | 360 |
| FREAITDFMF NGFSIPKGWK LYWSANSTHK NAECFPMPEK FDPTRFEGNG PAPYTFVPFG | 420 |
| GGPRMCPGKE YARLEILVFM HNLVKRFKWE KVIPDEKIIV DPFPIPAKDL PIRLYPHKA | 479 |

SEQ ID NO: 115
Artificial Sequence

| | |
|---|---|
| atggcctctg ttactttggg ttcctggatc gtcgtccacc accataacca tcaccatcca | 60 |
| tcatctatcc taactaaatc tcgttcaaga tcctgtccta ttacactaac caaaccaatc | 120 |
| tcttttcgtt caaagagaac agtttcctct agtagttcta tcgtgtcctc tagtgtcgtc | 180 |
| actaaggaag acaatctgag acagtctgaa ccttcttcct ttgatttcat gtcatatatc | 240 |
| attactaagg cagaactagt gaataaggct cttgattcag cagttccatt aagagagcca | 300 |
| ttgaaaatcc atgaagcaat gagatactct cttctagctg gcgggaagag agtcagacct | 360 |
| gtactctgca tagcagcgtg cgaattagtt ggtggcgagg aatcaaccgc tatgcctgcc | 420 |
| gcttgtgctg tagaaatgat tcatacaatg tcactgatac acgatgattt gccatgtatg | 480 |
| gataacgatg atctgagaag gggtaagcca actaaccata aggttttcgg cgaagatgtt | 540 |
| gccgtcttag ctggtgatgc tttgttatct ttcgcgttcg aacatttggc atccgcaaca | 600 |
| tcaagtgatg ttgtgtcacc agtaagagta gttagagcag ttggagaact ggctaaagct | 660 |
| attggaactg agggtttagt tgcaggtcaa gtcgtcgata tctcttccga aggtcttgat | 720 |
| ttgaatgatg taggtcttga acatctcgaa ttcatccatc ttcacaagac agctgcactt | 780 |
| ttagaagcca gtgcggttct cggcgcaatt gttggcggag ggagtgatga cgaaattgag | 840 |
| agattgagga agtttgctag atgtatagga ttactgttcc aagtagtaga cgatatacta | 900 |
| gatgtgacaa agtcttccaa agagttggga aaaacagctg gtaaagattt gattgccgac | 960 |
| aaattgacct accctaagat tatggggcta gaaaaatcaa gagaatttgc cgagaaactc | 1020 |
| aatagagagg cgcgtgatca actgttgggt ttcgattctg ataaagttgc accactctta | 1080 |
| gccttagcca actacatcgc ttacagacaa aactaa | 1116 |

SEQ ID NO: 116
Arabidopsis thaliana

| | |
|---|---|
| MASVTLGSWI VVHHHNHHHP SSILTKSRSR SCPITLTKPI SFRSKRTVSS SSSIVSSSVV | 60 |
| TKEDNLRQSE PSSFDFMSYI ITKAELVNKA LDSAVPLREP LKIHEAMRYS LLAGGKRVRP | 120 |
| VLCIAACELV GGEESTAMPA ACAVEMIHTM SLIHDDLPCM DNDDLRRGKP TNHKVFGEDV | 180 |
| AVLAGDALLS FAFEHLASAT SSDVVSPVRV VRAVGELAKA IGTEGLVAGQ VVDISSEGLD | 240 |
| LNDVGLEHLE FIHLHKTAAL LEASAVLGAI VGGGSDDEIE RLRKFARCIG LLFQVVDDIL | 300 |
| DVTKSSKELG KTAGKDLIAD KLTYPKIMGL EKSREFAEKL NREARDQLLG FDSDKVAPLL | 360 |
| ALANYIAYRQ N | 371 |

SEQ ID NO: 117
Rubus suavissimus

| | |
|---|---|
| MATLLEHFQA MPFAIPIALA ALSWLFLFYI KVSFFSNKSA QAKLPPVPVV PGLPVIGNLL | 60 |
| QLKEKKPYQT FTRWAEEYGP IYSIRTGAST MVVLNTTQVA KEAMVTRYLS ISTRKLSNAL | 120 |
| KILTADKCMV AISDYNDFHK MIKRYILSNV LGPSAQKRHR SNRDTLRANV CSRLHSQVKN | 180 |
| SPREAVNFRR VFEWELFGIA LKQAFGKDIE KPIYVEELGT TLSRDEIFKV LVLDIMEGAI | 240 |
| EVDWRDFFPY LRWIPNTRME TKIQRLYFRR KAVMTALINE QKKRIASGEE INCYIDFLLK | 300 |
| EGKTLTMDQI SMLLWETVIE TADTTMVTTE WAMYEVAKDS KRQDRLYQEI QKVCGSEMVT | 360 |
| EEYLSQLPYL NAVFHETLRK HSPAALVPLR YAHEDTQLGG YYIPAGTEIA INIYGCNMDK | 420 |
| HQWESPEEWK PERFLDPKFD PMDLYKTMAF GAGKRVCAGS LQAMLIACPT IGRLVQEFEW | 480 |
| KLRDGEEENV DTVGLTTHKR YPMHAILKPR S | 511 |

SEQ ID NO: 126
Arabidopsis thaliana

| | |
|---|---|
| atggcatcgg aatttcgtcc tcctcttcat tttgttctct tccctttcat ggctcaaggc | 60 |
| cacatgatcc caatggtaga tattgcaagg ctcctggctc agcgcggggt gactataacc | 120 |
| attgtcacta cacctcaaaa cgcaggccgg ttcaagaacg ttcttagccg ggctatccaa | 180 |
| tccggcttgc ccatcaatct cgtgcaagta aagtttccat ctcaagaatc gggttcaccg | 240 |
| gaaggacagg agaatttgga cttgctcgat tcattggggg cttcattaac cttcttcaaa | 300 |
| gcatttagcc tgctcgagga accagtcgag aagctcttga aagagattca acctaggcca | 360 |
| aactgcataa tcgctgacat gtgtttgcct tatacaaaca gaattgccaa gaatcttggt | 420 |
| ataccaaaaa tcatctttca tggcatgtgt tgcttcaatc ttctttgtac gcacataatg | 480 |
| caccaaaacc acgagttctt ggaaactata gagtctgaca aggaatactt ccccattcct | 540 |
| aatttccctg acagagttga gttcacaaaa tctcagcttc caatggtatt agttgctgga | 600 |
| gattggaaag acttccttga cggaatgaca gaaggggata acacttctta tggtgtgatt | 660 |
| gttaacacgt ttgaagagct cgagccagct tatgttagag actacaagaa ggttaaagcg | 720 |
| ggtaagatat ggagcatcgg accggtttcc ttgtgcaaca agttaggaga agaccaagct | 780 |
| gagaggggaa acaaggcgga cattgatcaa gacgagtgta ttaaatggct tgattctaaa | 840 |
| gaagaagggt cggtgctata tgtttgcctt ggaagtatat gcaatcttcc tctgtctcag | 900 |
| ctcaaagagc tcggcttagg cctcgaggaa tcccaaagac ctttcatttg ggtcataaga | 960 |
| ggttgggaga agtataacga gttacttgaa tggatctcag agagcggtta taaggaaaga | 1020 |
| atcaaagaaa gaggccttct cataacagga tggtcgcctc aaatgcttat ccttacacat | 1080 |
| cctgccgttg gaggattctt gacacattgt ggatggaact ctactcttga aggaatcact | 1140 |
| tcaggcgttc cattactcac gtggccactg tttggagacc aattctgcaa tgagaaattg | 1200 |
| gcggtgcaga tactaaaagc cggtgtgaga gctggggttg aagagtccat gagatgggga | 1260 |
| gaagaggaga aaataggagt actggtggat aaagaaggag taaagaaggc agtggaggaa | 1320 |
| ttgatgggtg atagtaatga tgctaaggag agaagaaaaa gagtgaaaga gcttggagaa | 1380 |
| ttagctcaca aggctgtgga agaaggaggc tcttctcatt ccaacatcac attcttgcta | 1440 |
| caagacataa tgcaattaga acaacccaag cgctag | 1476 |

SEQ ID NO: 127
Arabidopsis thaliana

| | |
|---|---|
| MASEFRPPLH FVLFPFMAQG HMIPMVDIAR LLAQRGVTIT IVTTPQNAGR FKNVLSRAIQ | 60 |
| SGLPINLVQV KFPSQESGSP EGQENLDLLD SLGASLTFFK AFSLLEEPVE KLLKEIQPRP | 120 |
| NCIIADMCLP YTNRIAKNLG IPKIIFHGMC CFNLLCTHIM HQNHEFLETI ESDKEYFPIP | 180 |
| NFPDRVEFTK SQLPMVLVAG DWKDFLDGMT EGDNTSYGVI VNTFEELEPA YVRDYKKVKA | 240 |
| GKIWSIGPVS LCNKLGEDQA ERGNKADIDQ DECIKWLDSK EEGSVLYVCL GSICNLPLSQ | 300 |
| LKELGLGLEE SQRPFIWVIR GWEKYNELLE WISESGYKER IKERGLLITG WSPQMLILTH | 360 |
| PAVGGFLTHC GWNSTLEGIT SGVPLLTWPL FGDQFCNEKL AVQILKAGVR AGVEESMRWG | 420 |
| EEEKIGVLVD KEGVKKAVEE LMGDSNDAKE RRKRVKELGE LAHKAVEEGG SSHSNITFLL | 480 |
| QDIMQLEQPK R | 491 |

SEQ ID NO: 132
Arabidopsis thaliana

| | |
|---|---|
| atggctacgg aaaaaaccca ccaatttcat ccttctcttc actttgtcct cttccctttc | 60 |
| atggctcaag gccacatgat tcccatgatt gatattgcaa gactcttggc tcagcgtggt | 120 |
| gtgaccataa caattgtcac gacacctcac aacgcagcaa ggtttaagaa tgtcctaaac | 180 |
| cgagcgatcg agtctggctt ggccatcaac atactgcatg tgaagtttcc atatcaagag | 240 |
| tttggtttgc cagaaggaaa agagaatata gattcgttag actcaacgga gttgatggta | 300 |
| cctttcttca aagcggtgaa cttgcttgaa gatccggtca tgaagctcat ggaagagatg | 360 |
| aaacctagac ctagctgtct aatttctgat tggtgtttgc cttatacaag cataatcgcc | 420 |
| aagaacttca atataccaaa gatagttttc cacggcatgg gttgctttaa tcttttgtgt | 480 |
| atgcatgttc tacgcagaaa cttagagatc ctagagaatg taaagtcgga tgaagagtat | 540 |
| ttcttggttc ctagttttcc tgatagagtt gaatttacaa agcttcaact tcctgtgaaa | 600 |
| gcaaatgcaa gtggagattg gaaagagata atggatgaaa tggtaaaagc agaatacaca | 660 |
| tcctatggtg tgatcgtcaa cacatttcag gagttggagc caccttatgt caaagactac | 720 |
| aaagaggcaa tggatggaaa agtatggtcc attggacccg tttccttgtg taacaaggca | 780 |
| ggtgcagaca aagctgagag gggaagcaag gccgccattg atcaagatga gtgtcttcaa | 840 |
| tggcttgatt ctaaagaaga aggttcggtg ctctatgttt gccttggaag tatatgtaat | 900 |
| cttcctttgt ctcagctcaa ggagctgggg ctaggccttg aggaatctcg aagatctttt | 960 |
| atttgggtca taagaggttc ggaaaagtat aaagaactat ttgagtggat gttggagagc | 1020 |
| ggttttgaag aaagaatcaa agagagagga cttctcatta aagggtgggc acctcaagtc | 1080 |
| cttatccttt cacatccttc cgttggagga ttcctgacac actgtggatg gaactcgact | 1140 |
| ctcgaaggaa tcacctcagg cattccactg atcacttggc cgctgtttgg agaccaattc | 1200 |
| tgcaaccaaa aactggtcgt tcaagtacta aaagccggtg taagtgccgg ggttgaagaa | 1260 |
| gtcatgaaat ggggagaaga agataaaata ggagtgttag tggataaaga aggagtgaaa | 1320 |
| aaggctgtgg aagaattgat gggtgatagt gatgatgcaa aagagaggag aagaagagtc | 1380 |
| aaagagcttg gagaattagc tcacaaagct gtggaaaaag gaggctcttc tcattctaac | 1440 |
| atcacactct tgctacaaga cataatgcaa ctagcacaat tcaagaattg a | 1491 |

SEQ ID NO: 133
Arabidopsis thaliana

| | |
|---|---|
| MATEKTHQFH PSLHFVLFPF MAQGHMIPMI DIARLLAQRG VTITIVTTPH NAARFKNVLN | 60 |
| RAIESGLAIN ILHVKFPYQE FGLPEGKENI DSLDSTELMV PFFKAVNLLE DPVMKLMEEM | 120 |
| KPRPSCLISD WCLPYTSIIA KNFNIPKIVF HGMGCFNLLC MHVLRRNLEI LENVKSDEEY | 180 |
| FLVPSFPDRV EFTKLQLPVK ANASGDWKEI MDEMVKAEYT SYGVIVNTFQ ELEPPYVKDY | 240 |
| KEAMDGKVWS IGPVSLCNKA GADKAERGSK AAIDQDECLQ WLDSKEEGSV LYVCLGSICN | 300 |
| LPLSQLKELG LGLEESRRSF IWVIRGSEKY KELFEWMLES GFEERIKERG LLIKGWAPQV | 360 |
| LILSHPSVGG FLTHCGWNST LEGITSGIPL ITWPLFGDQF CNQKLVVQVL KAGVSAGVEE | 420 |
| VMKWGEEDKI GVLVDKEGVK KAVEELMGDS DDAKERRRRV KELGELAHKA VEKGGSSHSN | 480 |
| ITLLLQDIMQ LAQFKN | 496 |

SEQ ID NO: 134
Arabidopsis thaliana

| | |
|---|---|
| atggtttccg aaacaaccaa atcttctcca cttcactttg ttctcttccc tttcatggct | 60 |
| caaggccaca tgattcccat ggttgatatt gcaaggctct tggctcagcg tggtgtgatc | 120 |
| ataacaattg tcacgacgcc tcacaatgca gcgaggttca agaatgtcct aaaccgtgcc | 180 |
| attgagtctg gcttgcccat caacttagtg caagtcaagt ttccatatct agaagctggt | 240 |
| ttgcaagaag gacaagagaa tatcgattct cttgacacaa tggagcggat gatacctttc | 300 |
| tttaaagcgg ttaactttct cgaagaacca gtccagaagc tcattgaaga gatgaaccct | 360 |
| cgaccaagct gtctaatttc tgatttttgt ttgccttata caagcaaaat cgccaagaag | 420 |
| ttcaatatcc caaagatcct cttccatggc atgggttgct tttgtcttct gtgtatgcat | 480 |
| gttttacgca agaaccgtga gatcttggac aatttaaagt cagataagga gcttttcact | 540 |
| gttcctgatt ttcctgatag agttgaattc acaagaacgc aagttccggt agaaacatat | 600 |
| gttccagctg gagactggaa agatatcttt gatggtatgg tagaagcgaa tgagacatct | 660 |
| tatggtgtga tcgtcaactc atttcaagag ctcgagcctg cttatgccaa agactacaag | 720 |
| gaggtaaggt ccggtaaagc atggaccatt ggacccgttt ccttgtgcaa caaggtagga | 780 |
| gccgacaaag cagagagggg aaacaaatca gacattgatc aagatgagtg ccttaaatgg | 840 |
| ctcgattcta agaaacatgg ctcggtgctt tacgtttgtc ttggaagtat ctgtaatctt | 900 |
| cctttgtctc aactcaagga gctgggacta ggcctagagg aatcccaaag acctttcatt | 960 |
| tgggtcataa gaggttggga gaagtacaaa gagttagttg agtggttctc ggaaagcggc | 1020 |
| tttgaagata gaatccaaga tagaggactt ctcatcaaag gatggtcccc tcaaatgctt | 1080 |
| atcctttcac atccatcagt tggagggttc ctaacacact gtggttggaa ctcgactctt | 1140 |
| gaggggataa ctgctggtct accgctactt acatggccgc tattcgcaga ccaattctgc | 1200 |
| aatgagaaat tggtcgttga ggtactaaaa gccggtgtaa gatccggggt tgaacagcct | 1260 |
| atgaaatggg gagaagagga gaaaatagga gtgttggtgg ataaagaagg agtgaagaag | 1320 |
| gcagtggaag aattaatggg tgagagtgat gatgcaaaag agagaagaag aagagccaaa | 1380 |
| gagcttggag attcagctca caaggctgtg gaagaaggag gctcttctca ttctaacatc | 1440 |
| tctttcttgc tacaagacat aatggaactg gcagaaccca ataattga | 1488 |

SEQ ID NO: 135
Arabidopsis thaliana

| | |
|---|---|
| MVSETTKSSP LHFVLFPFMA QGHMIPMVDI ARLLAQRGVI ITIVTTPHNA ARFKNVLNRA | 60 |
| IESGLPINLV QVKFPYLEAG LQEGQENIDS LDTMERMIPF FKAVNFLEEP VQKLIEEMNP | 120 |
| RPSCLISDFC LPYTSKIAKK FNIPKILFHG MGCFCLLCMH VLRKNREILD NLKSDKELFT | 180 |
| VPDFPDRVEF TRTQVPVETY VPAGDWKDIF DGMVEANETS YGVIVNSFQE LEPAYAKDYK | 240 |
| EVRSGKAWTI GPVSLCNKVG ADKAERGNKS DIDQDECLKW LDSKKHGSVL YVCLGSICNL | 300 |
| PLSQLKELGL GLEESQRPFI WVIRGWEKYK ELVEWFSESG FEDRIQDRGL LIKGWSPQML | 360 |
| ILSHPSVGGF LTHCGWNSTL EGITAGLPLL TWPLFADQFC NEKLVVEVLK AGVRSGVEQP | 420 |
| MKWGEEEKIG VLVDKEGVKK AVEELMGESD DAKERRRRAK ELGDSAHKAV EEGGSSHSNI | 480 |
| SFLLQDIMEL AEPNN | 495 |

SEQ ID NO: 136
Arabidopsis thaliana

| | |
|---|---|
| atggctttcg aaaaaaacaa cgaacctttt cctcttcact ttgttctctt ccctttcatg | 60 |
| gctcaaggcc acatgattcc catggttgat attgcaaggc tcttggctca gcgaggtgtg | 120 |
| cttataacaa ttgtcacgac gcctcacaat gcagcaaggt tcaagaatgt cctaaaccgt | 180 |
| gccattgagt ctggtttgcc catcaaccta gtgcaagtca agtttccata tcaagaagct | 240 |
| ggtctgcaag aaggacaaga aaatatggat ttgcttacca cgatggagca gataacatct | 300 |
| ttctttaaag cggttaactt actcaaagaa ccagtccaga accttattga agagatgagc | 360 |
| ccgcgaccaa gctgtctaat ctctgatatg tgtttgtcgt atacaagcga aatcgccaag | 420 |
| aagttcaaaa taccaaagat cctcttccat ggcatgggtt gcttttgtct tctgtgtgtt | 480 |
| aacgttctgc gcaagaaccg tgagatcttg gacaatttaa agtctgataa ggagtacttc | 540 |
| attgttcctt attttcctga tagagttgaa ttcacaagac ctcaagttcc ggtggaaaca | 600 |
| tatgttcctg caggctggaa agagatcttg gaggatatgg tagaagcgga taagacatct | 660 |
| tatggtgtta tagtcaactc atttcaagag ctcgaacctg cgtatgccaa agacttcaag | 720 |
| gaggcaaggt ctggtaaagc atggaccatt ggacctgttt ccttgtgcaa caaggtagga | 780 |
| gtagacaaag cagagagggg aaacaaatca gatattgatc aagatgagtg ccttgaatgg | 840 |
| ctcgattcta aggaaccggg atctgtgctc tacgtttgcc ttggaagtat ttgtaatctt | 900 |
| cctctgtctc agctccttga gctgggacta ggcctagagg aatcccaaag acctttcatc | 960 |
| tgggtcataa gaggttggga gaaatacaaa gagttagttg agtggttctc ggaaagcggc | 1020 |
| tttgaagata gaatccaaga tagaggactt ctcatcaaag gatggtcccc tcaaatgctt | 1080 |
| atcctttcac atccttctgt tggagggttc ttaacgcact gcggatggaa ctcgactctt | 1140 |
| gaggggataa ctgctggtct accaatgctt acatggccac tatttgcaga ccaattctgc | 1200 |
| aacgagaaac tggtcgtaca aatactaaaa gtcggtgtaa gtgccgaggt taaagaggtc | 1260 |
| atgaaatggg gagaagaaga gaagatagga gtgttggtgg ataaagaagg agtgaagaag | 1320 |
| gcagtggaag aactaatggg tgagagtgat gatgcaaaag agagaagaag aagagccaaa | 1380 |
| gagcttggag aatcagctca caaggctgtg gaagaaggag gctcctctca ttctaatatc | 1440 |
| actttcttgc tacaagacat aatgcaacta gcacagtcca ataattga | 1488 |

SEQ ID NO: 137
Arabidopsis thaliana

| | |
|---|---|
| MAFEKNNEPF PLHFVLFPFM AQGHMIPMVD IARLLAQRGV LITIVTTPHN AARFKNVLNR | 60 |
| AIESGLPINL VQVKFPYQEA GLQEGQENMD LLTTMEQITS FFKAVNLLKE PVQNLIEEMS | 120 |
| PRPSCLISDM CLSYTSEIAK KFKIPKILFH GMGCFCLLCV NVLRKNREIL DNLKSDKEYF | 180 |
| IVPYFPDRVE FTRPQVPVET YVPAGWKEIL EDMVEADKTS YGVIVNSFQE LEPAYAKDFK | 240 |
| EARSGKAWTI GPVSLCNKVG VDKAERGNKS DIDQDECLEW LDSKEPGSVL YVCLGSICNL | 300 |
| PLSQLLELGL GLEESQRPFI WVIRGWEKYK ELVEWFSESG FEDRIQDRGL LIKGWSPQML | 360 |
| ILSHPSVGGF LTHCGWNSTL EGITAGLPML TWPLFADQFC NEKLVVQILK VGVSAEVKEV | 420 |
| MKWGEEEKIG VLVDKEGVKK AVEELMGESD DAKERRRRAK ELGESAHKAV EEGGSSHSNI | 480 |
| TFLLQDIMQL AQSNN | 495 |

SEQ ID NO: 138
Arabidopsis thaliana

| | |
|---|---|
| atgtgttctc atgatcctct tcacttcgtc gtaataccct ttatggccca aggccatatg | 60 |
| atcccattgg tcgacatctc taggctcttg tcccagcgcc aaggcgtgac tgtctgcatc | 120 |
| atcacaacta ctcaaaatgt agccaagatc aagacttcac tctcattttc ctctttgttt | 180 |
| gcgactatca acatcgttga agttaagttt ctgtctcaac aaacgggttt gccagaaggg | 240 |
| tgcgagagtt tagatatgtt ggcttcaatg ggcgatatgg tgaagttctt tgatgctgcc | 300 |
| aactcacttg aggagcaagt tgagaaagct atggaagaga tggttcagcc gcggccaagc | 360 |
| tgcatcattg gagacatgag ccttcctttc acttcaagac ttgccaagaa attcaagatc | 420 |
| cccaaactta tcttccatgg gttttcttgt ttcagcctca tgtctataca agtggttcga | 480 |
| gaaagcggga tcttgaaaat gatagaatca aacgacgagt attttgattt gcccggcttg | 540 |
| cctgacaaag ttgagttcac gaaacctcag gtctctgtgt tgcaacctgt tgaaggaaat | 600 |
| atgaaagaga gtacggccaa gattattgaa gctgataatg actcttatgg tgttattgtg | 660 |
| aacacttttg aagagttaga ggttgattat gcaagagaat ataggaaagc aagggctgga | 720 |
| aaagtttggt gcgttggacc tgtttccttg tgcaataggt tagggttaga caaagctaaa | 780 |
| agaggagata aggcttctat tggtcaagac caatgtcttc aatggcttga ctctcaagaa | 840 |
| actggttcag tgctctacgt ttgccttgga agtctatgta atcttccctt ggctcagctc | 900 |
| aaagagctgg gactaggcct tgaggcatct aataaacctt tcatatgggt tataagagaa | 960 |
| tggggaaaat atggagattt agcaaattgg atgcaacaaa gcggatttga agagcggatc | 1020 |
| aaagatagag gactggtgat caaaggttgg gcgccgcaag ttttcatcct ctcacacgca | 1080 |
| tccattggag ggtttttgac tcactgtgga tggaactcga cactagaagg aattactgca | 1140 |
| ggagttccat tattgacatg gcctttgttt gctgaacaat tcttgaatga gaagttagtt | 1200 |
| gtgcagatac taaaagcagg gttaaagata ggagtagaga aattgatgaa atatggaaaa | 1260 |
| gaagaggaga taggagcgat ggtgagcaga gaatgtgtga gaaaagctgt ggatgagcta | 1320 |
| atgggtgata gtgaagaagc agaagagaga agaagaaaag ttacagaact tagtgacttg | 1380 |
| gcaaataagg ctttggaaaa aggaggatct tcagattcta atatcacatt gctcattcaa | 1440 |
| gatattatgg agcaatcaca aaatcaattc tag | 1473 |

SEQ ID NO: 139
Arabidopsis thaliana

| | |
|---|---|
| MCSHDPLHFV VIPFMAQGHM IPLVDISRLL SQRQGVTVCI ITTTQNVAKI KTSLSFSSLF | 60 |
| ATINIVEVKF LSQQTGLPEG CESLDMLASM GDMVKFFDAA NSLEEQVEKA MEEMVQPRPS | 120 |
| CIIGDMSLPF TSRLAKKFKI PKLIFHGFSC FSLMSIQVVR ESGILKMIES NDEYFDLPGL | 180 |
| PDKVEFTKPQ VSVLQPVEGN MKESTAKIIE ADNDSYGVIV NTFEELEVDY AREYRKARAG | 240 |
| KVWCVGPVSL CNRLGLDKAK RGDKASIGQD QCLQWLDSQE TGSVLYVCLG SLCNLPLAQL | 300 |
| KELGLGLEAS NKPFIWVIRE WGKYGDLANW MQQSGFEERI KDRGLVIKGW APQVFILSHA | 360 |
| SIGGFLTHCG WNSTLEGITA GVPLLTWPLF AEQFLNEKLV VQILKAGLKI GVEKLMKYGK | 420 |
| EEEIGAMVSR ECVRKAVDEL MGDSEEAEER RRKVTELSDL ANKALEKGGS SDSNITLLIQ | 480 |
| DIMEQSQNQF | 490 |

SEQ ID NO: 140
Stevia rebaudiana

| | |
|---|---|
| atgtcgccaa aaatggtggc accaccaacc aaccttcatt ttgttttgtt tcctcttatg | 60 |
| gctcaaggcc atctggtacc catggtcgac atcgctcgaa tcttagccca acgtggtgca | 120 |
| acggtcacca taatcaccac accctaccat gccaaccggg tcagaccggt tatctcccga | 180 |
| gccatcgcga ccaatctcaa gatccagcta ctcgaactcc aactgcggtc aaccgaagcc | 240 |
| ggtttacccg aagggtgcga aagcttcgac caacttccgt cattcgagta ctggaaaaat | 300 |
| atttcaaccg ctatcgattt gttacaacaa cccgctgaag atttgctccg agaactttca | 360 |
| ccaccacccg attgcatcat atcggacttt ttgttcccgt ggaccaccga tgtggctcga | 420 |
| cggttaaaca tcccccggct cgtgttcaat ggaccgggct gcttttatct cttgtgcatc | 480 |
| catgttgcga tcacttccaa cattttggga gagaatgaac cggtcagtag taataccgag | 540 |
| cgcgttgtgc tgcccggttt acctgaccgg atcgaagtca ctaaacttca gatcgtcggt | 600 |
| tcgtcgagac cagccaacgt agacgaaatg ggctcgtggc ttcgagccgt agaagctgag | 660 |
| aaagcttcat tcgggatagt ggttaatact ttcgaagagc ttgaaccgga gtacgttgaa | 720 |
| gaatacaaaa cggttaaaga taagaagatg tggtgtatcg gcccggtttc gttatgcaac | 780 |
| aaaaccgggc cggatttagc cgagcgagga aacaaagctg caataaccga acacaactgc | 840 |
| ttaaaatggc tcgatgagag aaaactgggg tccgtgttat acgtttgttt aggtagcctt | 900 |
| gcacgcattt ctgccgcaca agcaatcgag ctcgggttag gactcgagtc cataaaccgt | 960 |
| ccctttatat ggtgcgtaag aaacgaaacc gatgagctca aaacatggtt tttggatggg | 1020 |
| tttgaagaaa gggttagaga tcgcgggttg atcgttcatg gttgggcgcc acaggttttg | 1080 |
| atactgtcgc acccaaccat tggcggtttc ttaacccatt gcggttggaa ctcgactatt | 1140 |
| gaatcgatta ccgcgggtgt tccaatgatc acgtggccat tttttgcgga ccagtttttg | 1200 |
| aatgaagctt ttatagttga agttttgaag attggagtta ggattggtgt tgagagggct | 1260 |
| tgtttgtttg gggaagaaga taaggttgga gtgttggtga agaaggagga tgtgaagaag | 1320 |
| gctgttgaat gcttgatgga tgaagatgaa gatggtgatc agagaagaaa gagggtgatt | 1380 |
| gagcttgcaa aaatggcgaa gattgcaatg gcggaaggtg gatcttctta tgaaaatgta | 1440 |
| tcgtcgttga ttcgagatgt gactgaaaca gttagagcac cacattag | 1488 |

SEQ ID NO: 141
Stevia rebaudiana

| | |
|---|---|
| MSPKMVAPPT NLHFVLFPLM AQGHLVPMVD IARILAQRGA TVTIITTPYH ANRVRPVISR | 60 |
| AIATNLKIQL LELQLRSTEA GLPEGCESFD QLPSFEYWKN ISTAIDLLQQ PAEDLLRELS | 120 |
| PPPDCIISDF LFPWTTDVAR RLNIPRLVFN GPGCFYLLCI HVAITSNILG ENEPVSSNTE | 180 |
| RVVLPGLPDR IEVTKLQIVG SSRPANVDEM GSWLRAVEAE KASFGIVVNT FEELEPEYVE | 240 |
| EYKTVKDKKM WCIGPVSLCN KTGPDLAERG NKAAITEHNC LKWLDERKLG SVLYVCLGSL | 300 |
| ARISAAQAIE LGLGLESINR PFIWCVRNET DELKTWFLDG FEERVRDRGL IVHGWAPQVL | 360 |
| ILSHPTIGGF LTHCGWNSTI ESITAGVPMI TWPFFADQFL NEAFIVEVLK IGVRIGVERA | 420 |
| CLFGEEDKVG VLVKKEDVKK AVECLMDEDE DGDQRRKRVI ELAKMAKIAM AEGGSSYENV | 480 |
| SSLIRDVTET VRAPH | 495 |

SEQ ID NO: 142
Arabidopsis thaliana

| | |
|---|---|
| atgggagaga aagcgaaagc aaatgtgtta gtcttctcat ttccgataca aggtcacata | 60 |
| aaccctctcc tccaattctc aaaacgccta ctctctaaaa acgtcaacgt cacattcctc | 120 |
| accacttcct ccacccacaa ctccatcctc cgccgtgcca tcaccggcgg agccactgct | 180 |
| cttcctctct cttttgtccc cattgacgat ggattcgagg aagatcaccc atctacggac | 240 |
| acatctcccg actacttcgc aaagttccaa gaaaacgtat ctcgaagcct ctcagagctt | 300 |
| atctcctcga tggacccaaa accaaacgcc gtcgtttacg actcgtgcct gccttatgtc | 360 |
| ctcgacgttt gccggaaaca tcctggcgtt gctgcggcgt cgtttttcac tcagtcctcc | 420 |
| accgtgaacg cgacctatat tcatttcttg cgtggagagt ttaaggagtt tcaaaatgat | 480 |
| gtcgttttgc ctgcaatgcc tccgctgaag ggtaatgact taccggtgtt tctgtacgat | 540 |
| aacaatctct gccggccgtt gtttgagctc attagtagcc agttcgtgaa tgttgacgac | 600 |
| attgacttct tcttggttaa ctctttcgac gaactcgaag tcgaggtgct acaatggatg | 660 |
| aaaaaccaat ggccggtcaa gaacatagga ccgatgattc catcaatgta cttagacaaa | 720 |
| cgattagcag gtgacaaaga ctacggaatc aacctcttca atgcccaagt caacgaatgc | 780 |
| cttgattggc ttgactcaaa accgcccggt tcagtgatct acgtgtcttt tggaagcttg | 840 |
| gccgtcttaa aagacgatca aatgatagaa gtcgcggctg gtctaaaaca aactggccat | 900 |
| aacttcttat gggttgttag agaaactgaa acaaagaagc ttccaagcaa ttacatagag | 960 |
| gacatttgtg acaagggatt gatagtgaat tggagtcctc aattacaagt tcttgcacat | 1020 |
| aaatcaatcg gttgtttcat gactcattgc gggtggaatt cgactttaga ggcattgagc | 1080 |
| ttaggagttg ctttgatagg aatgccggct tatagcgacc agccgactaa tgctaagttt | 1140 |
| attgaagatg tgtggaaggt tggggttagg gttaaggcag atcaaaatgg gtttgttccg | 1200 |
| aaggaagaga ttgtgagatg tgttggagaa gttatggaag atatgtcgga gaaagggaag | 1260 |
| gagattagaa aaaatgctcg gaggttgatg gagtttgcaa gggaagcttt gtctgatgga | 1320 |
| ggaaattctg ataagaatat tgatgagttt gttgctaaaa ttgtgaggta a | 1371 |

SEQ ID NO: 143
Arabidopsis thaliana

| | |
|---|---|
| MGEKAKANVL VFSFPIQGHI NPLLQFSKRL LSKNVNVTFL TTSSTHNSIL RRAITGGATA | 60 |
| LPLSFVPIDD GFEEDHPSTD TSPDYFAKFQ ENVSRSLSEL ISSMDPKPNA VVYDSCLPYV | 120 |
| LDVCRKHPGV AAASFFTQSS TVNATYIHFL RGEFKEFQND VVLPAMPPLK GNDLPVFLYD | 180 |
| NNLCRPLFEL ISSQFVNVDD IDFFLVNSFD ELEVEVLQWM KNQWPVKNIG PMIPSMYLDK | 240 |
| RLAGDKDYGI NLFNAQVNEC LDWLDSKPPG SVIYVSFGSL AVLKDDQMIE VAAGLKQTGH | 300 |
| NFLWVVRETE TKKLPSNYIE DICDKGLIVN WSPQLQVLAH KSIGCFMTHC GWNSTLEALS | 360 |
| LGVALIGMPA YSDQPTNAKF IEDVWKVGVR VKADQNGFVP KEEIVRCVGE VMEDMSEKGK | 420 |
| EIRKNARRLM EFAREALSDG GNSDKNIDEF VAKIVR | 456 |

SEQ ID NO: 144
Arabidopsis thaliana

| | |
|---|---|
| atggcgccac cgcattttct actggtaacg tttccggcgc aaggtcacgt gaacccatct | 60 |
| ctccgttttg ctcgtcggct catcaaaaga accggcgcac gtgtcacttt cgtcacttgt | 120 |
| gtctccgtct tccacaactc catgatcgca aaccacaaca aagtcgaaaa tctctctttc | 180 |
| cttactttct ccgacggttt cgacgatgga ggcatttcca cctacgaaga ccgtcagaaa | 240 |
| aggtcggtga atctcaaggt taacggcgat aaggcactat cggatttcat cgaagctact | 300 |
| aagaatggtg actctcccgt gacttgcttg atctacacga ttcttctcaa ttgggctcca | 360 |
| aaagtagcac gtagatttca acttccctcc gctcttctct ggatccaacc ggctttggtt | 420 |
| ttcaacatct attacactca tttcatggga aacaagtccg ttttcgagtt acctaatctg | 480 |
| tcttctctgg aaatcagaga tcttccatct ttcctcacac cttccaacac aaacaaaggc | 540 |
| gcatacgatg cgtttcaaga aatgatggag tttctcataa aagaaaccaa accgaaaatt | 600 |
| ctcatcaaca ctttcgattc gctggaacca gaggccttaa cggctttccc gaatatcgat | 660 |
| atggtggcgg ttggtccttt acttcccacg gagattttct caggaagcac caacaaatca | 720 |
| gttaaagatc aaagtagtag ttatacactt tggctagact cgaaaacaga gtcctctgtt | 780 |
| atttacgttt cctttggaac aatggttgag ttgtccaaga aacagataga ggaactagcg | 840 |
| agagcactca tagaagggaa acgaccgttt ttgtgggtta taactgataa atccaacaga | 900 |
| gaaacgaaaa cagaaggaga agaagagaca gagattgaga agatagctgg attcagacac | 960 |
| gagcttgaag aggttgggat gattgtgtcg tggtgttcgc agatagaggt tttaagtcac | 1020 |
| cgagccgtag gttgttttgt gactcattgt gggtggagct cgacgctgga gagtttggtt | 1080 |
| cttggcgttc cggttgtggc gtttccgatg tggtcggatc aaccgacgaa cgcgaagcta | 1140 |
| ctggaagaaa gttggaagac tggtgtgagg gtaagagaga acaaggatgg tttggtggag | 1200 |
| agaggagaga tcaggaggtg tttggaagcc gtgatggagg agaagtcggt ggagttgagg | 1260 |
| gaaaacgcaa agaaatggaa gcgtttagcg atggaagcgg gtagagaagg aggatcttcg | 1320 |
| gataagaaca tggaggcttt tgtggaggat atttgtggag aatctcttat tcaaaacttg | 1380 |
| tgtgaagcag aggaggtaaa agtacgctag | 1410 |

SEQ ID NO: 145
Arabidopsis thaliana

| | |
|---|---|
| MAPPHFLLVT FPAQGHVNPS LRFARRLIKR TGARVTFVTC VSVFHNSMIA NHNKVENLSF | 60 |
| LTFSDGFDDG GISTYEDRQK RSVNLKVNGD KALSDFIEAT KNGDSPVTCL IYTILLNWAP | 120 |
| KVARRFQLPS ALLWIQPALV FNIYYTHFMG NKSVFELPNL SSLEIRDLPS FLTPSNTNKG | 180 |
| AYDAFQEMME FLIKETKPKI LINTFDSLEP EALTAFPNID MVAVGPLLPT EIFSGSTNKS | 240 |
| VKDQSSSYTL WLDSKTESSV IYVSFGTMVE LSKKQIEELA RALIEGKRPF LWVITDKSNR | 300 |
| ETKTEGEEET EIEKIAGFRH ELEEVGMIVS WCSQIEVLSH RAVGCFVTHC GWSSTLESLV | 360 |
| LGVPVVAFPM WSDQPTNAKL LEESWKTGVR VRENKDGLVE RGEIRRCLEA VMEEKSVELR | 420 |
| ENAKKWKRLA MEAGREGGSS DKNMEAFVED ICGESLIQNL CEAEEVKVR | 469 |

SEQ ID NO: 146
Gardenia jasminoides

| | |
|---|---|
| atggttcaac aaagacacgt tttgttgatt acctatccag ctcaaggtca tattaaccca | 60 |
| gctttacaat tcgcccaaag attattgaga atgggtatcc aagttacctt ggctacttct | 120 |
| gtttatgcct tgtccagaat gaagaagtca tctggttcta ctccaaaggg tttgactttt | 180 |
| gctactttct ctgatggtta cgatgatggt tttagaccta agggtgttga tcacaccgaa | 240 |
| tatatgtcat ctttggctaa gcaaggttcc aacactttga gaaacgttat taacacctct | 300 |
| gctgatcaag gttgtccagt tacttgtttg gtttacactt tgttgttgcc atgggctgct | 360 |
| actgttgcta gagaatgtca tattccatct gccttgttgt ggattcaacc agttgctgtt | 420 |
| atggacatct attactacta cttcagaggt tacgaagatg acgtcaagaa caattctaat | 480 |
| gatccaacct ggtccattca atttccaggt ttgccatcta tgaaggctaa agatttgcct | 540 |
| tcctttatct tgccatcctc cgataatatc tactcttttg ctttgccaac cttcaagaag | 600 |
| caattggaaa ctttggacga agaagaaaga ccaaaggttt tggttaatac cttcgatgct | 660 |
| ttggaaccac aagccttgaa agctattgaa tcttacaact tgattgccat cggtccattg | 720 |
| actccatctg cttttttgga tggtaaagat ccatccgaaa catccttttc tggtgacttg | 780 |
| tttcaaaagt ccaaggacta caaagaatgg ttgaactcta gaccagcagg ttctgttgtt | 840 |
| tacgtttctt ttggttcctt gttgaccttg ccaaagcaac aaatggaaga aattgctaga | 900 |
| ggtttgttga agtctggtag accatttttg tgggttatca gagctaaaga aaacggtgaa | 960 |
| gaagaaaaag aagaagatag attgatctgc atggaagaat tggaagaaca aggtatgata | 1020 |
| gttccatggt gctcccaaat tgaagttttg actcatccat ctttgggttg cttcgttact | 1080 |
| cattgtggtt ggaatagtac tttggaaacc ttggtttgtg gtgttccagt tgttgcattt | 1140 |
| ccacattgga ccgatcaagg tactaatgcc aaattgattg aagatgtttg ggaaaccggt | 1200 |
| gttagagttg ttccaaatga agatggtact gtcgaatctg acgaaatcaa gagatgtatc | 1260 |
| gaaaccgtta tggatgatgg tgaaaaaggt gtcgaattga agagaaatgc caagaagtgg | 1320 |
| aaagaattgg ctagagaagc tatgcaagaa gatggttctt ctgacaagaa tttgaaggct | 1380 |
| ttcgttgaag atgctggtaa aggttatcaa gccgaatcta actga | 1425 |

SEQ ID NO: 147
Gardenia jasminoides

| | |
|---|---|
| MVQQRHVLLI TYPAQGHINP ALQFAQRLLR MGIQVTLATS VYALSRMKKS SGSTPKGLTF | 60 |
| ATFSDGYDDG FRPKGVDHTE YMSSLAKQGS NTLRNVINTS ADQGCPVTCL VYTLLLPWAA | 120 |
| TVARECHIPS ALLWIQPVAV MDIYYYYFRG YEDDVKNNSN DPTWSIQFPG LPSMKAKDLP | 180 |
| SFILPSSDNI YSFALPTFKK QLETLDEEER PKVLVNTFDA LEPQALKAIE SYNLIAIGPL | 240 |
| TPSAFLDGKD PSETSFSGDL FQKSKDYKEW LNSRPAGSVV YVSFGSLLTL PKQQMEEIAR | 300 |
| GLLKSGRPFL WVIRAKENGE EEKEEDRLIC MEELEEQGMI VPWCSQIEVL THPSLGCFVT | 360 |
| HCGWNSTLET LVCGVPVVAF PHWTDQGTNA KLIEDVWETG VRVVPNEDGT VESDEIKRCI | 420 |
| ETVMDDGEKG VELKRNAKKW KELAREAMQE DGSSDKNLKA FVEDAGKGYQ AESN | 474 |

SEQ ID NO: 152
Arabidopsis thaliana

| | |
|---|---|
| atggaggaaa agcctgcaag gagaagcgta gtgttggttc catttccagc acaaggacat | 60 |
| atatctccaa tgatgcaact tgccaaaacc cttcacttaa agggtttctc gatcacagtt | 120 |
| gttcagacta agttcaatta ctttagccct tcagatgact tcactcatga ttttcagttc | 180 |
| gtcaccattc cagaaagctt accagagtct gatttcaaga atctcggacc aatacagttt | 240 |
| ctgtttaagc tcaacaaaga gtgtaaggtg agcttcaagg actgtttggg tcagttggtg | 300 |
| ctgcaacaaa gtaatgagat ctcatgtgtc atctacgatg agttcatgta ctttgctgaa | 360 |
| gctgcagcca aagagtgtaa gcttccaaac atcattttca gcacaacaag tgccacggct | 420 |
| ttcgcttgcc gctctgtatt tgacaaacta tatgcaaaca atgtccaagc tcccttgaaa | 480 |
| gaaactaaag gacaacaaga agagctagtt ccggagtttt atcccttgag atataaagac | 540 |
| tttccagttt cacggtttgc atcattagag agcataatgg aggtgtatag gaatacagtt | 600 |
| gacaaacgga cagcttcctc ggtgataatc aacactgcga gctgtctaga gagctcatct | 660 |
| ctgtcttttc tgcaacaaca acagctacaa attccagtgt atcctatagg ccctcttcac | 720 |
| atggtggcct cagctcctac aagtctgctt gaagagaaca agagctgcat cgaatggttg | 780 |
| aacaaacaaa aggtaaactc ggtgatatac ataagcatgg gaagcatagc tttaatggaa | 840 |
| atcaacgaga taatggaagt cgcgtcagga ttggctgcta gcaaccaaca cttcttatgg | 900 |
| gtgatccgac cagggtcaat acctggttcc gagtggatag agtccatgcc tgaagagttt | 960 |
| agtaagatgg ttttggaccg aggttacatt gtgaaatggg ctccacagaa ggaagtactt | 1020 |
| tctcatcctg cagtaggagg gttttggagc cattgtggat ggaactcgac actagaaagc | 1080 |
| atcggccaag gagttccaat gatctgcagg ccattttcgg gtgatcaaaa ggtgaacgct | 1140 |
| agatacttgg agtgtgtatg gaaaattggg attcaagtgg agggtgagct agacagagga | 1200 |
| gtggtcgaga gagctgtgaa gaggttaatg gttgacgaag aaggagagga gatgaggaag | 1260 |
| agagctttca gtttaaaaga gcaacttaga gcctctgtta aaagtggagg ctcttcacac | 1320 |
| aactcgctag aagagtttgt acacttcata aggactgcct ag | 1362 |

SEQ ID NO: 153
Arabidopsis thaliana

| | |
|---|---|
| MEEKPARRSV VLVPFPAQGH ISPMMQLAKT LHLKGFSITV VQTKFNYFSP SDDFTHDFQF | 60 |
| VTIPESLPES DFKNLGPIQF LFKLNKECKV SFKDCLGQLV LQQSNEISCV IYDEFMYFAE | 120 |
| AAAKECKLPN IIFSTTSATA FACRSVFDKL YANNVQAPLK ETKGQQEELV PEFYPLRYKD | 180 |
| FPVSRFASLE SIMEVYRNTV DKRTASSVII NTASCLESSS LSFLQQQQLQ IPVYPIGPLH | 240 |
| MVASAPTSLL EENKSCIEWL NKQKVNSVIY ISMGSIALME INEIMEVASG LAASNQHFLW | 300 |
| VIRPGSIPGS EWIESMPEEF SKMVLDRGYI VKWAPQKEVL SHPAVGGFWS HCGWNSTLES | 360 |
| IGQGVPMICR PFSGDQKVNA RYLECVWKIG IQVEGELDRG VVERAVKRLM VDEEGEEMRK | 420 |
| RAFSLKEQLR ASVKSGGSSH NSLEEFVHFI RTA | 453 |

SEQ ID NO: 168
Catharanthus roseus

| | |
|---|---|
| atggcaactg aacaacaaca agcatctatc tcctgcaaaa tcttaatgtt tccttggtta | 60 |
| gccttcggtc atatctcttc tttcttacaa ttggctaaga aattgtctga tagaggtttc | 120 |
| tacttctaca tttgtagtac tccaattaat ttggactcta ttaaaaataa gataaaccaa | 180 |
| aactattctt catccataca attggttgat ttgcatttgc caaacagtcc tcaattgcca | 240 |
| ccttctttac atactacaaa tggtttgcca cctcacttaa tgtctacatt gaaaaacgct | 300 |
| ttgatcgatg caaatccaga cttatgcaag attatagcct caattaaacc agatttgatc | 360 |
| atctatgact tacatcaacc ttggaccgaa gcattggctt ctagacacaa cattcctgct | 420 |
| gttagttttt ctactatgaa tgccgtatcc tttgcttacg ttatgcacat gttcatgaat | 480 |
| ccaggtatag aatttccttt caaagcaatc cacttatcag attttgaaca agccagattc | 540 |
| ttggaacaat tagaatcagc taagaacgat gcctccgcta aagacccaga attgcaaggt | 600 |
| agtaagggtt tctttaactc taccttcatt gttagaagtt ctagagaaat cgagggtaaa | 660 |
| tacgttgatt acttgtcaga aatcttaaag tccaaggtca ttccagtatg tcctgttata | 720 |
| tctttgaata acaacgatca aggtcagggt aacaaagatg aagacgaaat aatccaatgg | 780 |
| ttagacaaaa agtctcatag atcatccgta tttgtttcat tcggttccga atactttttg | 840 |
| aacatgcaag aaatcgaaga aatcgctata ggtttggaat tatctaacgt caactttata | 900 |
| tgggtattga gattcccaaa gggtgaagat acaaaaattg aagaagtttt gcctgaaggt | 960 |
| ttcttggaca gagttaaaac caagggtaga attgtccacg gttgggcacc acaagccaga | 1020 |
| atcttgggtc atccttcaat tggtggtttc gtatcccact gcggttggaa tagtgttatg | 1080 |
| gaatctatcc aaatcggtgt cccaattata gcaatgccta tgaacttgga tcaacctttt | 1140 |
| aatgccagat tagttgtcga aatcggtgtc ggtattgaag taggtagaga tgaaaacggt | 1200 |
| aaattaaaga gagaaagaat cggtgaagtt atcaaggaag tcgctatagg taaaaagggt | 1260 |
| gaaaaattga gaaagacagc aaaagatttg ggtcaaaaat tgagagatag agaaaaacaa | 1320 |
| gactttgacg aattagcagc aactttgaaa caattatgcg tatga | 1365 |

SEQ ID NO: 169
Catharanthus roseus

| | |
|---|---|
| MATEQQQASI SCKILMFPWL AFGHISSFLQ LAKKLSDRGF YFYICSTPIN LDSIKNKINQ | 60 |
| NYSSSIQLVD LHLPNSPQLP PSLHTTNGLP PHLMSTLKNA LIDANPDLCK IIASIKPDLI | 120 |
| IYDLHQPWTE ALASRHNIPA VSFSTMNAVS FAYVMHMFMN PGIEFPFKAI HLSDFEQARF | 180 |
| LEQLESAKND ASAKDPELQG SKGFFNSTFI VRSSREIEGK YVDYLSEILK SKVIPVCPVI | 240 |
| SLNNNDQGQG NKDEDEIIQW LDKKSHRSSV FVSFGSEYFL NMQEIEEIAI GLELSNVNFI | 300 |
| WVLRFPKGED TKIEEVLPEG FLDRVKTKGR IVHGWAPQAR ILGHPSIGGF VSHCGWNSVM | 360 |
| ESIQIGVPII AMPMNLDQPF NARLVVEIGV GIEVGRDENG KLKRERIGEV IKEVAIGKKG | 420 |
| EKLRKTAKDL GQKLRDREKQ DFDELAATLK QLCV | 454 |

SEQ ID NO: 172
Arabidopsis thaliana

| | |
|---|---|
| atgaccaaat tctccgagcc aatcagagac tcccacgtgg cagttctcgc gtttttcccc | 60 |
| gttggcgctc atgccggtcc tctcttagcc gtcactcgcc gtctcgccgc cgcttctccc | 120 |
| tccaccatct tttctttctt caacaccgca agatcaaacg cgtcgttgtt ctcctctgat | 180 |
| catcccgaga acatcaaggt ccacgacgtc tctgacggtg ttccggaggg aaccatgctc | 240 |
| gggaatccac tggagatggt cgagctgttt ctcgaagcgg ctccacgtat tttccggagc | 300 |
| gaaatcgcgg cggcagagat agaagttgga aagaaagtga catgcatgct aacagatgcc | 360 |
| ttcttctggt tcgcagcgga catagcggct gagctgaacg cgacttgggt tgccttctgg | 420 |
| gccggcggag caaactcact ctgtgctcat ctctacactg atctcatcag agaaaccatc | 480 |
| ggtctcaaag atgtgagtat ggaagagaca ttagggttta taccaggaat ggagaattac | 540 |
| agagttaaag atataccaga ggaagttgta tttgaagatt tggactctgt tttcccaaag | 600 |
| gctttatacc aaatgagtct tgctttacct cgtgcctctg ctgttttcat cagttccttt | 660 |
| gaagagttag aacctacatt gaactataac ctaagatcca aacttaaacg tttcttgaac | 720 |
| atcgcccctc tcacgttatt atcttctaca tcggagaaag agatgcgtga tcctcatggc | 780 |
| tgctttgctt ggatggggaa gagatcagct gcttctgtag cgtacattag cttcggcacc | 840 |
| gtcatggaac ctcctcctga agagcttgtg gcgatagcac aagggttgga atcaagcaaa | 900 |
| gtgccgtttg tttggtcgct gaaggagaag aacatggttc atctaccaaa agggtttttg | 960 |
| gatcggacaa gagagcaagg gatagtggtt ccttgggctc cacaagtgga actgctgaaa | 1020 |
| cacgaggcaa tgggtgtgaa tgtgacacat tgtggatgga actcagtgtt ggagagtgtg | 1080 |
| tcggcaggtg taccgatgat cggcagaccg attttggcgg ataataggct caacggaaga | 1140 |
| gcagtggagg ttgtgtggaa ggttggagtg atgatggata atggagtctt cacgaaagaa | 1200 |
| ggatttgaga agtgtttgaa tgatgttttt gttcatgatg atggtaagac gatgaaggct | 1260 |
| aatgccaaga agcttaaaga aaaactccaa gaagatttct ccatgaaagg aagctcttta | 1320 |
| gagaatttca aaatattgtt ggacgaaatt gtgaaagttt ag | 1362 |

SEQ ID NO: 173
Arabidopsis thaliana

| | |
|---|---|
| MTKFSEPIRD SHVAVLAFFP VGAHAGPLLA VTRRLAAASP STIFSFFNTA RSNASLFSSD | 60 |
| HPENIKVHDV SDGVPEGTML GNPLEMVELF LEAAPRIFRS EIAAAEIEVG KKVTCMLTDA | 120 |
| FFWFAADIAA ELNATWVAFW AGGANSLCAH LYTDLIRETI GLKDVSMEET LGFIPGMENY | 180 |
| RVKDIPEEVV FEDLDSVFPK ALYQMSLALP RASAVFISSF EELEPTLNYN LRSKLKRFLN | 240 |
| IAPLTLLSST SEKEMRDPHG CFAWMGKRSA ASVAYISFGT VMEPPPEELV AIAQGLESSK | 300 |
| VPFVWSLKEK NMVHLPKGFL DRTREQGIVV PWAPQVELLK HEAMGVNVTH CGWNSVLESV | 360 |
| SAGVPMIGRP ILADNRLNGR AVEVVWKVGV MMDNGVFTKE GFEKCLNDVF VHDDGKTMKA | 420 |
| NAKKLKEKLQ EDFSMKGSSL ENFKILLDEI VKV | 453 |

SEQ ID NO: 176
Streptomyces antibioticus

| | |
|---|---|
| atgacttctg aacatagatc cgcttccgtt actccaagac atatttcatt cttcaacatc | 60 |
| ccaggtcatg gtcatgttaa tccatctttg ggtatcgttc aagaattggt tgctagaggt | 120 |
| cacagagttt cttacgctat taccgatgaa tttgctgctc aagttaaggc tgctggtgct | 180 |
| actccagttg tttatgattc catcttgcca aaagaatcca acccagaaga atcttggcca | 240 |
| gaagatcaag aatctgctat gggtttgttc ttggatgaag ctgttagagt cttgccacaa | 300 |
| ttagaagatg cttacgctga tgatagacca gatttgatcg tttacgatat tgcttcttgg | 360 |
| ccagctccag ttttgggtag aaaatgggat attccattcg tccaattatc cccaactttc | 420 |
| gttgcttacg aaggttttga agaagatgtt ccagcagttc aagatccaac tgctgataga | 480 |
| ggtgaagaag ctgctgctcc agcaggtact ggtgatgctg aagaaggtgc tgaagctgaa | 540 |
| gatggtttgg ttagattctt cactagattg tccgctttct tggaagaaca tggtgttgat | 600 |
| actccagcta ccgaattttt gattgctcca aacagatgca tcgttgcttt gccaagaact | 660 |
| tttcaaatca agggtgatac cgttggtgat aactacactt ttgttggtcc aacttacggt | 720 |
| gatagatctc atcaaggtac ttgggaaggt ccaggtgatg gtagaccagt tttgttgatt | 780 |
| gctttgggtt ctgctttcac tgatcacttg gatttctaca gaacctgttt gtctgctgtt | 840 |
| gatggtttgg attggcatgt tgttttgtct gttggtagat ttgttgatcc agcagatttg | 900 |
| ggtgaagttc caccaaatgt tgaagttcat caatgggttc cacaattaga tattttgacc | 960 |
| aaggcttccg ccttcattac tcatgctggt atgggttcta ctatggaagc cttgtctaat | 1020 |
| gctgttccaa tggttgctgt tccacaaatt gctgaacaaa ctatgaacgc cgaaagaata | 1080 |
| gtcgaattgg gtttgggtag acatatccca agagatcaag ttactgccga aaaattgaga | 1140 |
| gaagctgttt tggctgttgc ttctgatcca ggtgttgctg aaagattggc tgctgttaga | 1200 |
| caagaaatta gagaagccgg tggtgctaga gctgctgctg atattttgga aggtattttg | 1260 |
| gctgaagccg gttaa | 1275 |

SEQ ID NO: 177
Streptomyces antibioticus

| | |
|---|---|
| MTSEHRSASV TPRHISFFNI PGHGHVNPSL GIVQELVARG HRVSYAITDE FAAQVKAAGA | 60 |
| TPVVYDSILP KESNPEESWP EDQESAMGLF LDEAVRVLPQ LEDAYADDRP DLIVYDIASW | 120 |
| PAPVLGRKWD IPFVQLSPTF VAYEGFEEDV PAVQDPTADR GEEAAAPAGT GDAEEGAEAE | 180 |
| DGLVRFFTRL SAFLEEHGVD TPATEFLIAP NRCIVALPRT FQIKGDTVGD NYTFVGPTYG | 240 |
| DRSHQGTWEG PGDGRPVLLI ALGSAFTDHL DFYRTCLSAV DGLDWHVVLS VGRFVDPADL | 300 |
| GEVPPNVEVH QWVPQLDILT KASAFITHAG MGSTMEALSN AVPMVAVPQI AEQTMNAERI | 360 |
| VELGLGRHIP RDQVTAEKLR EAVLAVASDP GVAERLAAVR QEIREAGGAR AAADILEGIL | 420 |
| AEAG | 424 |

SEQ ID NO: 180
Oryza sativa

| | |
|---|---|
| atgaagcaaa ccgtcgtcct gtaccccggc ggcggcgtcg gccacgtcgt ccccatgctg | 60 |
| gagctcgcca aggtcttcgt caagcacggg cacgacgtca ccatggtgct gctggagccg | 120 |
| cccttcaagt cgtccgactc cggcgccctc gccgtcgagc gcctcgtcgc ctccaaccct | 180 |
| tccgtctcct tccacgtcct cccgccactc cccgcccccg acttcgccag cttcggcaag | 240 |
| cacccgttcc tcctcgtcat ccagctcctg cgccagtaca acgagcggct cgagagcttc | 300 |
| ctcctctcca tccctcgaca gcgcctgcac tccctcgtca tcgacatgtt ctgcgtcgac | 360 |
| gccatcgacg tgtgcgcaaa gctcggcgtg ccggtgtaca cgttcttcgc ctcgggcgtc | 420 |
| tcggtgctgt ccgtcttgac ccagctccca ccgtttcttg ccggtaggga gacgggcctg | 480 |
| aaggagcttg gcgacacgcc gcttgatttc ctcggtgttt cgccgatgcc ggcgtctcat | 540 |
| ctcgtcaagg aattgctcga gcatccggag gacgagttgt gcaaggccat ggtgaaccgc | 600 |
| tgggagcgca acacggaaac catgggcgtc ctggtgaact cgttcgaatc gttggagagc | 660 |
| cgggcggctc aggcgctcag ggacgacccg ctctgcgtcc caggcaaggt gctgcctccg | 720 |
| atctactgcg tcgggccttt ggtcggcggc ggcgcggagg aggcggccga gaggcacgag | 780 |
| tgcctcgtct ggctcgacgc tcagccggag cacagcgtcg tgttcctctg cttcgggagc | 840 |
| aagggcgtgt tctccgcgga gcagctcaag gagatcgccg tcggcttgga gaactccagg | 900 |
| caacggttca tgtgggtcgt gcgcacgccg ccgacaacca ccgaaggctt gaagaagtac | 960 |
| ttcgagcaac gcgcggcgcc ggacctcgac gcgctcttcc cggatgggtt cgtggagcgt | 1020 |
| accaaggacc gtggcttcat cgtcacgacg tgggcgccgc aggtggacgt gctccgccac | 1080 |
| cgggcgaccg gcgcgttcgt gacgcactgc gggtggaact cggcgctgga gggcatcacg | 1140 |
| gcgggggtgc cgatgctgtg ctggccgcag tacgcggagc agaagatgaa caaggtgttc | 1200 |
| atgacggcgg agatgggcgt cggggtggag ctggacgggt acaactcgga ctttgtcaaa | 1260 |
| gcggaggagt tggaggccaa ggtgaggctg gtgatggagt cggaggaagg gaagcagctc | 1320 |
| agggctcgtt cggctgcgcg gaagaaggag gcagaggcgg cgctggagga agggggctcg | 1380 |
| tcgcacgctg cgttcgtcca gttcctgtcc gatgtggaga atcttgtcca gaactaa | 1437 |

SEQ ID NO: 181
Oryza sativa

| | |
|---|---|
| MKQTVVLYPG GGVGHVVPML ELAKVFVKHG HDVTMVLLEP PFKSSDSGAL AVERLVASNP | 60 |
| SVSFHVLPPL PAPDFASFGK HPFLLVIQLL RQYNERLESF LLSIPRQRLH SLVIDMFCVD | 120 |
| AIDVCAKLGV PVYTFFASGV SVLSVLTQLP PFLAGRETGL KELGDTPLDF LGVSPMPASH | 180 |
| LVKELLEHPE DELCKAMVNR WERNTETMGV LVNSFESLES RAAQALRDDP LCVPGKVLPP | 240 |
| IYCVGPLVGG GAEEAAERHE CLVWLDAQPE HSVVFLCFGS KGVFSAEQLK EIAVGLENSR | 300 |
| QRFMWVVRTP PTTTEGLKKY FEQRAAPDLD ALFPDGFVER TKDRGFIVTT WAPQVDVLRH | 360 |
| RATGAFVTHC GWNSALEGIT AGVPMLCWPQ YAEQKMNKVF MTAEMGVGVE LDGYNSDFVK | 420 |
| AEELEAKVRL VMESEEGKQL RARSAARKKE AEAALEEGGS SHAAFVQFLS DVENLVQN | 478 |

SEQ ID NO: 182
Nicotiana tabacum

| | |
|---|---|
| atgactactc aaaaagctca ttgcttgatc ttaccatatc cagctcaggg tcatatcaac | 60 |
| cctatgctcc aattctccaa acgtttgcaa tccaaaggtg tcaaaatcac tatagcagcc | 120 |
| accaaatcat tcttgaaaac catgcaagaa ttgtcaactt ctgtgtcagt cgaggctatc | 180 |
| tccgatggct atgatgatgg cggacgcgag caagctggaa cctttgtggc ctatattaca | 240 |
| agattcaaag aagttggctc ggatactttg tctcagctta ttggaaagtt aacaaattgt | 300 |
| ggttgtcctg tgagttgcat agtttacgat ccatttcttc cttgggctgt tgaagtggga | 360 |
| aataattttg gagtagctac tgctgctttt ttcactcaat cttgtgcagt ggataacatt | 420 |
| tattaccatg tacataaagg ggttctaaaa cttcctccaa ctgacgttga taaagaaatc | 480 |
| tcaattcctg gattattaac aattgaggca tcagatgtac ctagttttgt ttctaatcct | 540 |
| gaatcttcaa gaatacttga aatgttggtg aatcagttct cgaatcttga gaacacagat | 600 |
| tgggtcctaa tcaacagttt ctatgaattg gagaaagagg taattgattg gatggccaag | 660 |
| atctatccaa tcaagacaat tggaccaact ataccatcaa tgtacctaga caagaggcta | 720 |
| ccagatgaca aagaatatgg ccttagtgtc ttcaagccaa tgacaaatgc atgcctaaac | 780 |
| tggttaaacc atcaaccagt tagctcagta gtatatgtat catttggaag tttagccaaa | 840 |
| ttagaagcag agcaaatgga agaattagca tggggtttga gtaatagcaa caagaacttc | 900 |
| ttgtgggtag ttagatccac tgaagaatcc aaacttccca acaacttttt agaggaatta | 960 |
| gcaagtgaaa aaggattagt cgtgtcatgg tgtccacaat tacaagtctt ggaacataaa | 1020 |
| tcaatagggt gttttctcac gcactgtggc tggaattcaa ctttggaagc aattagtttg | 1080 |
| ggagtaccaa tgattgcaat gccacattgg tcagaccagc caacaaatgc gaagcttgtg | 1140 |
| gaagatgttt gggagatggg aattagacca aaacaagatg aaaaaggatt agttagaaga | 1200 |
| gaagttattg aagaatgtat taagatagtg atggaggaaa agaaaggaaa aaagattagg | 1260 |
| gaaaatgcaa agaaatggaa ggaattggct aggaaagctg tggatgaagg aggaagttca | 1320 |
| gatagaaata ttgaagaatt tgtttccaag ttggtgacta ttgcctcagt ggaaagctaa | 1380 |

SEQ ID NO: 183
Nicotiana tabacum

| | |
|---|---|
| MTTQKAHCLI LPYPAQGHIN PMLQFSKRLQ SKGVKITIAA TKSFLKTMQE LSTSVSVEAI | 60 |
| SDGYDDGGRE QAGTFVAYIT RFKEVGSDTL SQLIGKLTNC GCPVSCIVYD PFLPWAVEVG | 120 |
| NNFGVATAAF FTQSCAVDNI YYHVHKGVLK LPPTDVDKEI SIPGLLTIEA SDVPSFVSNP | 180 |
| ESSRILEMLV NQFSNLENTD WVLINSFYEL EKEVIDWMAK IYPIKTIGPT IPSMYLDKRL | 240 |
| PDDKEYGLSV FKPMTNACLN WLNHQPVSSV VYVSFGSLAK LEAEQMEELA WGLSNSNKNF | 300 |
| LWVVRSTEES KLPNNFLEEL ASEKGLVVSW CPQLQVLEHK SIGCFLTHCG WNSTLEAISL | 360 |
| GVPMIAMPHW SDQPTNAKLV EDVWEMGIRP KQDEKGLVRR EVIEECIKIV MEEKKGKKIR | 420 |
| ENAKKWKELA RKAVDEGGSS DRNIEEFVSK LVTIASVES | 459 |

SEQ ID NO: 184
Siraitia grosvenorii

| | |
|---|---|
| atggagaaag gcgatacgca tattctagtg tttcctttcc cttcacaagg ccacataaac | 60 |
| cctcttcttc aactatcgaa gcgcctaatc gccaagggaa tcaaggtttc gctggtcaca | 120 |
| accttacatg ttagcaatca cttgcagttg cagggtgctt attccaactc cgtgaagatc | 180 |
| gaagtcattt ccgatggctc tgaggatcgt ctggaaaccg atactatgcg ccaaactctg | 240 |
| gatcgatttc ggcagaagat gacgaagaac ttggaagatt tcttgcagaa agccatggtt | 300 |
| tcttcaaatc cgcctaaatt cattctgtat gattcgacaa tgccgtgggt tttggaggtc | 360 |
| gccaaggagt tcggactcga tagggccccg ttctacactc agtcttgtgc gcttaacagt | 420 |
| atcaattatc atgttcttca tggtcaattg aagcttcctc ctgaaacccc cacgatttcg | 480 |
| ttgccttcta tgcctctgct tcgccccagc gatctcccgg cttatgattt tgatcctgcc | 540 |
| tccactgaca ccatcatcga tcttcttacc agtcagtatt ctaatatcca ggatgcaaat | 600 |
| ctgcttttct gcaacacttt tgacaagttg gaaggcgaga ttatccaatg gatggagacc | 660 |
| ctgggtcgcc ctgtgaaaac cgtaggacca actgttccat cagcctactt agacaaaagg | 720 |
| gtagagaacg acaagcacta tgggctgagt ctgttcaagc ccaacgagga cgtctgcctc | 780 |
| aaatggcttg atagcaagcc ctctggttct gttctgtatg tgtcttatgg cagtttggtt | 840 |
| gaaatggggg aagagcagct gaaggagttg gctctgggaa tcaaggaaac tggcaagttc | 900 |
| ttcttgtggg tggtgagaga cactgaagca gagaagcttc ctcccaactt tgtggagagt | 960 |
| gtggcagaga aggggcttgt ggtcagctgg tgctcccagc tggaggtatt ggctcacccc | 1020 |
| tccgtcggct gcttcttcac gcactgtggc tggaactcga cgcttgaggc gctgtgcttg | 1080 |
| ggcgtcccgg tggtcgcttt cccacagtgg gctgatcagg taaccaatgc aaagttttta | 1140 |
| gaagatgttt ggaaggttgg gaagagggtg aagcggaatg agcagaggct ggcaagtaaa | 1200 |
| gaagaagtaa ggagttgcat ttgggaagtg atggagggag agagagccag cgagttcaag | 1260 |
| agcaactcca tggagtggaa gaagtgggca aaagaagctg tggatgaagg tgggagctct | 1320 |
| gataagaaca ttgaggagtt tgtggctatg ctcaagcaaa cttga | 1365 |

SEQ ID NO: 185
Siraitia grosvenorii

| | |
|---|---|
| MEKGDTHILV FPFPSQGHIN PLLQLSKRLI AKGIKVSLVT TLHVSNHLQL QGAYSNSVKI | 60 |
| EVISDGSEDR LETDTMRQTL DRFRQKMTKN LEDFLQKAMV SSNPPKFILY DSTMPWVLEV | 120 |
| AKEFGLDRAP FYTQSCALNS INYHVLHGQL KLPPETPTIS LPSMPLLRPS DLPAYDFDPA | 180 |
| STDTIIDLLT SQYSNIQDAN LLFCNTFDKL EGEIIQWMET LGRPVKTVGP TVPSAYLDKR | 240 |
| VENDKHYGLS LFKPNEDVCL KWLDSKPSGS VLYVSYGSLV EMGEEQLKEL ALGIKETGKF | 300 |
| FLWVVRDTEA EKLPPNFVES VAEKGLVVSW CSQLEVLAHP SVGCFFTHCG WNSTLEALCL | 360 |
| GVPVVAFPQW ADQVTNAKFL EDVWKVGKRV KRNEQRLASK EEVRSCIWEV MEGERASEFK | 420 |
| SNSMEWKKWA KEAVDEGGSS DKNIEEFVAM LKQT | 454 |

SEQ ID NO: 198
Crocus sativus

| | |
|---|---|
| atggggtcag aagataggtc cttgtccatc ttattctttc cttttatggc acaaggtcac | 60 |
| atgttaccta tgctagatat ggctaagtta tttgctctgt atggtgtcaa atcaacagta | 120 |
| gtgaccactc cagctaatgt accaatagtc aactcagtaa ttgatcagcc tgatgtttct | 180 |
| actttgcacc caatccaatt acgactgata ccatttccat ctgacacggg cttgcctgaa | 240 |
| ggttgtgaaa acgtatcatc aattcctcca agagacatgc caactgttca tgtcactttc | 300 |
| ttcagcgcta cagcaaaact tagagaacct tttggtaagg tgctagagga tctaagacca | 360 |
| gattgtattg ttactgacat gtttttccct tggacctacg atgtggccgc agaattaggt | 420 |
| atcccaagga ttgttttcca tgggacaaat ttcttttctc tctgcgtaac agattctctt | 480 |
| gaaagatata aaccagttga aaacttgcga agtgatgccg agtctgtagt gatcccagga | 540 |
| ctcccacaca gaatcgaggt attgcgttct caaataccag aatacgaaaa atcaaaagca | 600 |
| gattttgtta gagaagttag ggaatcagaa tctaagtctt acggagcggt ggttaattct | 660 |
| ttctttgaat tggaacctga ctacgctaga cattacagag aggttgtcgg cagacgtgct | 720 |
| tggcatatcg ggccacttgc tctggtcaat aactctacta cagacaaaag ctcaagagga | 780 |
| tacaagacag cgatcgatag aaacgattgt ttgaaatggc tcgattctaa aagactaaga | 840 |
| tccgttgtat atgtgtgctt tggctcaatg tctgactttt ccgatgccca attacgtgaa | 900 |
| atggcaagtg gtctagaggc atccaatcat cctttcattt gggtggttag aaaatctggc | 960 |
| aaggaatggt taccagaagg atttgaggaa agagtccagg agagaggttt gattatcaga | 1020 |
| ggctgggctc cacaaatctt aatactcaac catagagcag tgggaggctt catgacccat | 1080 |
| tgtgggtgga atagtagttt ggaagcagtt tctgccggac tgcctcttgt tacatggcct | 1140 |
| ctatttgcag aacaatttta caatgaaaga ttcatggttg atgttttgag aattggtgta | 1200 |
| tcagtgggtg cgaagagaca cggtatgaaa gccgaagaga gagaagtcgt agaagccaaa | 1260 |
| atggttaagg aagctgttga tggcttgatg gacgacggtg aagaggctga gggtagaagg | 1320 |
| cgtagagcta gagaactggg cgaaaaagct agaaaggccg tcgaaaaagg tggttcatcc | 1380 |
| tacgaggaca tgagaaatct tttgcaagag cttaagggtg atagcaagtt aactgtcgga | 1440 |
| tgctaa | 1446 |

SEQ ID NO: 199
Crocus sativus

| | |
|---|---|
| MGSEDRSLSI LFFPFMAQGH MLPMLDMAKL FALYGVKSTV VTTPANVPIV NSVIDQPDVS | 60 |
| TLHPIQLRLI PFPSDTGLPE GCENVSSIPP RDMPTVHVTF FSATAKLREP FGKVLEDLRP | 120 |
| DCIVTDMFFP WTYDVAAELG IPRIVFHGTN FFSLCVTDSL ERYKPVENLR SDAESVVIPG | 180 |
| LPHRIEVLRS QIPEYEKSKA DFVREVRESE SKSYGAVVNS FFELEPDYAR HYREVVGRRA | 240 |
| WHIGPLALVN NSTTDKSSRG YKTAIDRNDC LKWLDSKRLR SVVYVCFGSM SDFSDAQLRE | 300 |
| MASGLEASNH PFIWVVRKSG KEWLPEGFEE RVQERGLIIR GWAPQILILN HRAVGGFMTH | 360 |
| CGWNSSLEAV SAGLPLVTWP LFAEQFYNER FMVDVLRIGV SVGAKRHGMK AEEREVVEAK | 420 |
| MVKEAVDGLM DDGEEAEGRR RRARELGEKA RKAVEKGGSS YEDMRNLLQE LKGDSKLTVG | 480 |
| C | 481 |

SEQ ID NO: 200
Crocus sativus

| | |
|---|---|
| atggaggctg gaggtgacaa acttcacatt gttgtctttc catggttagc ttttggccac | 60 |
| atgttgccat ttctagagct gtctaagtct ttggctaaaa gaggtcactt aatcagtttt | 120 |
| gtttctacac ctaaaaacat tcaaagattt cctaatcttc caccacaaat ctcaccactt | 180 |
| atcaacttta tcccattaag tctacctaaa gtggagggca tgccaggtga cgtagaagct | 240 |
| accacagacc taccacctgc caacctacaa tatctgaaaa aggcacttga cgggttagaa | 300 |
| caacctttca gatcattcct aagagaggcc tccccaaaac ctgattggat aatccaagat | 360 |
| cttttacaac attggatacc tccaattgcc gcagaacttc atgttccttc catgtacttt | 420 |
| ggcacagtgc cagctgccgc cttgaccttt ttcggtcatc catcacaact tagttcaaga | 480 |
| gggaagggat tggaaggctg gctggcttca ccaccatggg ttccattccc atctaaggtg | 540 |
| gcatacagat tgcacgaact aatcgttatg gctaaagatg ccgctggtcc attgcattcc | 600 |
| ggtatgactg atgctagaag gatggaagct gcaatagttg gatgctgtgc agtcgctatt | 660 |
| agaacatgta gagaattgga atcagaatgg ttacctattc tggaggagat ctacggaaag | 720 |
| cctgtgatac cagttggatt acttttacct actgctgatg aatctactga tggaaactct | 780 |
| atcatagact ggttaggcac aagatcccag gaatcagtag tgtacattgc tctgggttca | 840 |
| gaagtttcta ttggtgtgga attgatacat gaattggcct tgggtcttga attagcaggt | 900 |
| ttgccattcc tatgggcact acgtagacct tatggactgt ctagtgatac tgagattttg | 960 |
| cctggtggat tcgaggagag aactagaggc tatggaaagg tagtcatggg ctgggttcct | 1020 |
| caaatgagag tcttggcaga tcgttctgta ggcggctttg tcacacactg tggttggtca | 1080 |
| tctgtagttg aatcattaca ttttgggcat ccactagttt tactgccaat cttcggtgac | 1140 |
| caaggattga atgcaagatt gctggaggaa aagggaattg gggtcgaagt agaaaggaag | 1200 |
| ggtgatgggt cttttacccg taatgaagtt gcaaaagcaa tcaatttgat catggtcgaa | 1260 |
| ggtgacggtt ctggttcctc ctacaggaaa aaggcaaagg aaatgaaaaa gattttcgct | 1320 |
| gataaggaat gccaggagaa atacgtggat gaatttgtgc agttcctgtt atcaaatggt | 1380 |
| actgctaaag gctaa | 1395 |

SEQ ID NO: 201
Crocus sativus

| | |
|---|---|
| MEAGGDKLHI VVFPWLAFGH MLPFLELSKS LAKRGHLISF VSTPKNIQRF PNLPPQISPL | 60 |
| INFIPLSLPK VEGMPGDVEA TTDLPPANLQ YLKKALDGLE QPFRSFLREA SPKPDWIIQD | 120 |
| LLQHWIPPIA AELHVPSMYF GTVPAAALTF FGHPSQLSSR GKGLEGWLAS PPWVPFPSKV | 180 |
| AYRLHELIVM AKDAAGPLHS GMTDARRMEA AIVGCCAVAI RTCRELESEW LPILEEIYGK | 240 |
| PVIPVGLLLP TADESTDGNS IIDWLGTRSQ ESVVYIALGS EVSIGVELIH ELALGLELAG | 300 |
| LPFLWALRRP YGLSSDTEIL PGGFEERTRG YGKVVMGWVP QMRVLADRSV GGFVTHCGWS | 360 |
| SVVESLHFGH PLVLLPIFGD QGLNARLLEE KGIGVEVERK GDGSFTRNEV AKAINLIMVE | 420 |
| GDGSGSSYRK KAKEMKKIFA DKECQEKYVD EFVQFLLSNG TAKG | 464 |

SEQ ID NO: 202
Arabidopsis thaliana

| | |
|---|---|
| atggagaaga tgagaggaca tgtattagca gtgccatttc caagccaagg acacatcacc | 60 |
| ccgattcgcc aattctgcaa acgacttcac tccaaaggtt tcaaaaccac tcacactctc | 120 |
| accactttta tcttcaacac aatccacctc gacccatcta gtcctatctc catagccaca | 180 |
| atctccgatg gctatgacca gggagggttc tcatcagccg gttctgtccc ggagtaccta | 240 |
| caaaacttca aaaccttcgg ctccaaaacc gtcgctgata tcatccgcaa acaccagagt | 300 |
| actgataacc ctattacttg tatcgtctat gattctttca tgccttgggc gcttgacctt | 360 |
| gcaatggatt ttggtctagc tgcggctcct ttcttcacgc agtcttgcgc cgttaactat | 420 |
| atcaattatc tttcttacat aaacaatggt agcttgacac ttcccatcaa ggatttgcct | 480 |
| cttcttgagc tccaagattt gcctactttc gtcactccta ctggttcaca ccttgcttac | 540 |
| tttgagatgg tgcttcaaca gttcaccaac ttcgacaaag ctgatttcgt actcgttaat | 600 |
| tccttccatg acctcgacct tcatgaagag gagttgttgt cgaaagtatg tcctgtgttg | 660 |
| acaattggtc caactgttcc atcaatgtac ttagaccaac agatcaaatc agacaacgac | 720 |
| tatgatctga acctctttga cttaaaagaa gctgccttat gcactgactg gctagacaag | 780 |
| aggccagaag gatcggtagt atatatagct tttgggagca tggctaaact gagtagtgag | 840 |
| cagatggaag agattgcttc ggcgataagc aacttcagct acctctgggt tgtcagagct | 900 |
| tcagaggagt caaagctccc accagggttt cttgaaacag tggataaaga caagagcttg | 960 |
| gtcttgaagt ggagtcctca gcttcaagtt ctgtcaaaca aagccatcgg ttgtttcatg | 1020 |
| actcactgtg gctggaactc aaccatggag ggtttgagtt taggggttcc catggtggct | 1080 |
| atgcctcaat ggactgatca accaatgaat gcaaagtata tacaagatgt atggaaggtt | 1140 |
| ggggttcgtg tgaaagcaga gaaagaaagt ggcatttgca aaagagagga gattgagttt | 1200 |
| agcatcaagg aagtgatgga aggagagaag agcaaagaga tgaaagagaa tgcgggaaaa | 1260 |
| tggagagact tggctgtgaa gtcactcagt gaaggaggtt ctacagatat caacattaac | 1320 |
| gaatttgtat caaaaattca aatcaaataa | 1350 |

SEQ ID NO: 203
Arabidopsis thaliana

| | |
|---|---|
| MEKMRGHVLA VPFPSQGHIT PIRQFCKRLH SKGFKTTHTL TTFIFNTIHL DPSSPISIAT | 60 |
| ISDGYDQGGF SSAGSVPEYL QNFKTFGSKT VADIIRKHQS TDNPITCIVY DSFMPWALDL | 120 |
| AMDFGLAAAP FFTQSCAVNY INYLSYINNG SLTLPIKDLP LLELQDLPTF VTPTGSHLAY | 180 |
| FEMVLQQFTN FDKADFVLVN SFHDLDLHEE ELLSKVCPVL TIGPTVPSMY LDQQIKSDND | 240 |
| YDLNLFDLKE AALCTDWLDK RPEGSVVYIA FGSMAKLSSE QMEEIASAIS NFSYLWVVRA | 300 |
| SEESKLPPGF LETVDKDKSL VLKWSPQLQV LSNKAIGCFM THCGWNSTME GLSLGVPMVA | 360 |
| MPQWTDQPMN AKYIQDVWKV GVRVKAEKES GICKREEIEF SIKEVMEGEK SKEMKENAGK | 420 |
| WRDLAVKSLS EGGSTDININ EFVSKIQIK | 449 |

SEQ ID NO: 204
Arabidopsis thaliana

| | |
|---|---|
| atggccaaca acaattccaa ctctcccacc ggtccacact ttctattcgt aacatttcca | 60 |
| gcccaaggtc acatcaaccc atctctcgag ctagccaaac gcctcgccgg aacaatctct | 120 |
| ggtgctcgag tcaccttcgc cgcctcaatc tctgcctaca accgccgcat gttctctaca | 180 |
| gaaaacgtcc ccgaaaccct aatcttcgct acctactccg atggccacga cgacggtttc | 240 |
| aaatcctctg cttactccga caaatctcgt caagacgcca ctggaaactt catgtctgag | 300 |
| atgagacgac gtggcaaaga gacactaacc gaactaatcg aagataaccg gaaacaaaac | 360 |
| aggcctttta cttgcgtggt ttacacgatt ctcctcactt gggtcgctga gctagcgcgt | 420 |
| gagtttcatc ttccttctgc tcttctttgg gtccaaccag taacagtctt ctccattttt | 480 |
| taccattact tcaatggcta cgaagatgca atctcagaga tggctaatac cccctctagt | 540 |
| tctattaaat taccttctct gccactgctt actgtccgtg atattccttc tttcattgtc | 600 |
| tcttccaatg tctacgcgtt tcttctaccc gcgtttcgag aacagattga ttcactgaag | 660 |
| gaagaaataa accctaagat cctcatcaac actttccaag agcttgagcc agaagccatg | 720 |
| agctcggttc cagataattt caagattgtc cctgtcggtc cgttactaac gttgagaacg | 780 |
| gatttttcga gtcgcggtga atacatagag tggttggata ctaaagcgga ttcgtctgtg | 840 |
| ctttatgttt cgttcgggac gcttgccgtg ttgagcaaga aacagcttgt ggagctttgt | 900 |
| aaagcgttga tacaaagtcg gagaccattc ttgtgggtga ttacggataa gtcgtacaga | 960 |
| aataaagaag atgagcaaga gaaggaagaa gattgcataa gtagtttcag agaagagctc | 1020 |
| gatgagatag gaatggtggt ttcatggtgt gatcagttta gggttttgaa tcatagatcg | 1080 |
| ataggttgtt tcgtgacgca ttgcgggtgg aactctacgc tggagagctt ggtttcagga | 1140 |
| gttccggtgg tggcgtttcc gcaatggaat gatcagatga tgaacgcgaa gcttttagaa | 1200 |
| gattgttgga aaacaggtgt aagagtgatg gagaagaagg aagaagaagg agttgtggtg | 1260 |
| gtggatagtg aggagatacg gcggtgcatt gaggaagtta tggaagacaa ggcggaggag | 1320 |
| tttagaggaa atgccacgag gtggaaggat ttagcggcgg aggctgtgag agaaggaggc | 1380 |
| tcttccttta atcatctcaa agcttttgtc gatgagcaca tctag | 1425 |

SEQ ID NO: 205
Arabidopsis thaliana

| | |
|---|---|
| MANNNSNSPT GPHFLFVTFP AQGHINPSLE LAKRLAGTIS GARVTFAASI SAYNRRMFST | 60 |
| ENVPETLIFA TYSDGHDDGF KSSAYSDKSR QDATGNFMSE MRRRGKETLT ELIEDNRKQN | 120 |
| RPFTCVVYTI LLTWVAELAR EFHLPSALLW VQPVTVFSIF YHYFNGYEDA ISEMANTPSS | 180 |
| SIKLPSLPLL TVRDIPSFIV SSNVYAFLLP AFREQIDSLK EEINPKILIN TFQELEPEAM | 240 |
| SSVPDNFKIV PVGPLLTLRT DFSSRGEYIE WLDTKADSSV LYVSFGTLAV LSKKQLVELC | 300 |
| KALIQSRRPF LWVITDKSYR NKEDEQEKEE DCISSFREEL DEIGMVVSWC DQFRVLNHRS | 360 |
| IGCFVTHCGW NSTLESLVSG VPVVAFPQWN DQMMNAKLLE DCWKTGVRVM EKKEEEGVVV | 420 |
| VDSEEIRRCI EEVMEDKAEE FRGNATRWKD LAAEAVREGG SSFNHLKAFV DEHI | 474 |

SEQ ID NO: 206
Arabidopsis thaliana

| | |
|---|---|
| atgggaagta atgagggtca agaaacacat gtcctaatgg tagcattagc attccaaggt | 60 |
| catctcaatc caatgctcaa attcgcaaaa catctcgcac gaaccaatct acacttcact | 120 |
| ctcgccacca ctgagcaagc ccgtgacctc ctctcttcca ccgctgacga acctcataga | 180 |
| ccggtggacc tcgctttctt ctcagacggt ctacctaaag acgatccaag agatcccgac | 240 |
| actctcgcaa agtcattgaa aaaagatgga gccaagaact tgtcaaaaat catcgaagaa | 300 |
| aagagatttg attgcatcat ctctgtgcct tttactccct gggttccagc tgttgcagct | 360 |
| gcacataaca ttccttgtgc aatcctctgg atccaagctt gtggagcttt ttctgtttat | 420 |
| taccgttatt acatgaagac aaatcctttc cccgaccttg aagatctgaa tcaaacagtg | 480 |
| gagttaccag ctttaccatt gttggaagtc cgagatctcc cgtcattgat gttaccttct | 540 |
| caaggagcta atgtcaatac cctaatggcg gaatttgcag attgtttgaa agatgtgaaa | 600 |
| tgggttttgg ttaactcgtt ttacgaactc gaatcagaga tcatcgagtc tatgtctgat | 660 |
| ttaaaaccta taatcccaat tggtcctctt gtttctccat tcctgttggg aaatgatgaa | 720 |
| gaaaaaaccc tagatatgtg gaaagttgat gattattgta tggagtggct tgacaagcaa | 780 |
| gctaggtctt cagttgttta catatctttc ggaagcatac tcaaatcatt ggagaatcaa | 840 |
| gttgagacca tagcaacggc attaaaaaac agaggagttc catttctttg ggtgatacgg | 900 |
| ccgaaggaga aaggcgaaaa cgtccaggtt ttgcaggaga tggttaaaga aggtaaaggg | 960 |
| gttgtaactg aatggggtca acaagaaaag atattgagcc acatggcgat ttcttgcttc | 1020 |
| atcacgcatt gtggatggaa ctcgacgatc gagacggtgg tgactggtgt tcccgtggtg | 1080 |
| gcgtatccga cttggataga tcagccgctt gatgcgagac tgcttgtgga tgtgtttgga | 1140 |
| atcggagtaa ggatgaagaa cgacgctatc gatggagagc ttaaggttgc agaggtggag | 1200 |
| agatgcattg aggccgtgac agagggacct gccgccgcgg atatgaggag gagagcgacg | 1260 |
| gagctgaagc acgccgcaag atcggcgatg tcacctggtg gatcttccgc tcagaattta | 1320 |
| gactcgttca ttagtgatat cccaatcact tga | 1353 |

SEQ ID NO: 207
Arabidopsis thaliana

| | |
|---|---|
| MGSNEGQETH VLMVALAFQG HLNPMLKFAK HLARTNLHFT LATTEQARDL LSSTADEPHR | 60 |
| PVDLAFFSDG LPKDDPRDPD TLAKSLKKDG AKNLSKIIEE KRFDCIISVP FTPWVPAVAA | 120 |
| AHNIPCAILW IQACGAFSVY YRYYMKTNPF PDLEDLNQTV ELPALPLLEV RDLPSLMLPS | 180 |
| QGANVNTLMA EFADCLKDVK WVLVNSFYEL ESEIIESMSD LKPIIPIGPL VSPFLLGNDE | 240 |
| EKTLDMWKVD DYCMEWLDKQ ARSSVVYISF GSILKSLENQ VETIATALKN RGVPFLWVIR | 300 |
| PKEKGENVQV LQEMVKEGKG VVTEWGQQEK ILSHMAISCF ITHCGWNSTI ETVVTGVPVV | 360 |
| AYPTWIDQPL DARLLVDVFG IGVRMKNDAI DGELKVAEVE RCIEAVTEGP AAADMRRRAT | 420 |
| ELKHAARSAM SPGGSSAQNL DSFISDIPIT | 450 |

SEQ ID NO: 208
Catharanthus roseus

| | |
|---|---|
| atggttaatc agctccatat tttcaacttc ccattcatgg cacagggcca tatgttaccc | 60 |
| gccttagaca tggccaatct attcacttct cgtggagtca aagtaacatt aatcacaacc | 120 |
| catcaacatg ttcccatgtt tacaaaatcc atagaaagga gcagaaattc tggatttgat | 180 |
| atatccattc aatccatcaa attcccagct tcagaagttg gtttacctga aggaatcgaa | 240 |
| agtctagatc aagtttcagg ggacgacgaa atgcttccta agttcatgag aggagttaat | 300 |
| ttactccaac aacctctcga acaactattg caagaatctc gtcctcattg tcttctttct | 360 |
| gatatgttct tcccttggac tactgaatct gctgctaaat ttggtattcc cagattgctt | 420 |
| tttcatgggt cctgttcctt tgccctctct gcagctgaaa gtgtgagaag aaataaacct | 480 |
| ttcgagaatg tttccacaga cacagaggaa tttgttgtgc ctgatcttcc ccaccaaatt | 540 |
| aaattaacca gaacacaaat ttcaacatac gaaagggaaa atattgagtc agattttacc | 600 |
| aaaatgctga agaaagttag ggattcagaa tccacatctt acggagttgt agtcaatagt | 660 |
| ttctatgaac ttgaaccaga ttatgccgat tattacatca acgttttggg aagaaaagca | 720 |
| tggcatatag ggcctttttt gctttgtaac aaatcacgag ctgaagataa agcccaaagg | 780 |
| gggaagaaat cagcaattga tgcagacgaa tgtttaaatt ggcttgattc gaaacaacca | 840 |
| aattccgtaa tttatctctg tttcggaagt atggccaatt taaattctgc ccaattacac | 900 |
| gaaattgcaa cagcccttga atcctccggc caaaatttca tctgggttgt tagaaaatgt | 960 |
| gtggacgaag aaaacagttc aaaatggttt ccagaaggat tcgaagaaag aacaaaagaa | 1020 |
| aaagggctaa ttataaaggg atgggcacca caaaccctaa ttcttgaaca cgaatcagta | 1080 |
| ggagcatttg ttacccattg tggttggaat tcaactcttg aaggaatctg cgcaggggtt | 1140 |
| cctctggtga cttggccttt ctttgctgag caatttttca atgagaaatt gattacagag | 1200 |
| gtactgaaaa cgggatacgg agttggggct cggcaatgga gtagagtttc aacagagatt | 1260 |
| ataaaaggag aagccatagc taatgctatt aatcgagtaa tggtgggtga tgaagctgtt | 1320 |
| gagatgagaa acagagcaaa agatttgaag gaaaaggcaa gaaaagcttt ggaagaagat | 1380 |
| ggatcttctt atcgtgatct tactgctctt attgaagaat tgggggcata tcgttctcaa | 1440 |
| gttgaaagaa agcaacaaga ctag | 1464 |

SEQ ID NO: 209
Catharanthus roseus

| | |
|---|---|
| MVNQLHIFNF PFMAQGHMLP ALDMANLFTS RGVKVTLITT HQHVPMFTKS IERSRNSGFD | 60 |
| ISIQSIKFPA SEVGLPEGIE SLDQVSGDDE MLPKFMRGVN LLQQPLEQLL QESRPHCLLS | 120 |
| DMFFPWTTES AAKFGIPRLL FHGSCSFALS AAESVRRNKP FENVSTDTEE FVVPDLPHQI | 180 |
| KLTRTQISTY ERENIESDFT KMLKKVRDSE STSYGVVVNS FYELEPDYAD YYINVLGRKA | 240 |
| WHIGPFLLCN KSRAEDKAQR GKKSAIDADE CLNWLDSKQP NSVIYLCFGS MANLNSAQLH | 300 |
| EIATALESSG QNFIWVVRKC VDEENSSKWF PEGFEERTKE KGLIIKGWAP QTLILEHESV | 360 |
| GAFVTHCGWN STLEGICAGV PLVTWPFFAE QFFNEKLITE VLKTGYGVGA RQWSRVSTEI | 420 |
| IKGEAIANAI NRVMVGDEAV EMRNRAKDLK EKARKALEED GSSYRDLTAL IEELGAYRSQ | 480 |
| VERKQQD | 487 |

SEQ ID NO: 210
Solanum lycopersicum

| | |
|---|---|
| atgactactc acaaagctca ttgcttaatt ttgccatttc caggccaagg tcatatcaac | 60 |
| ccaatgcttc aattctccaa acgtttacaa tccaaacgcg ttaaaatcac tatagcactc | 120 |
| acaaaatcct gtttgaaaac aatgcaagaa ttgtcaactt cagtatcaat cgaggcgatt | 180 |
| tctgatggct acgatgatgg tggtttccat caagcagaaa atttcgtagc ctacataaca | 240 |
| cgattcaaag aagttggttc ggatactctg tctcagctta ttaaaaaatt ggaaaatagt | 300 |
| gattgtcctg taaattgcat agtatatgat ccattcattc cttgggctgt tgaagttgca | 360 |
| aaacaatttg gattaattag tgctgcattt ttcacacaaa attgtgtagt ggataatctt | 420 |
| tattaccatg tacataaagg ggtgataaaa cttccaccta ctcaaaatga cgaagaaata | 480 |
| ttaattcctg gatttccaaa ttcgatcgat gcatcagatg taccttcttt tgttattagt | 540 |
| cctgaagcag aaaggatagt tgaaatgtta gcaaatcaat tctcaaatct tgacaaagtt | 600 |
| gattatgttc taatcaatag cttctatgag ttggagaaag aggtaaatga atggatgtca | 660 |
| aagatatatc caataaagac aattggacca acaataccat caatgtactt agacaagaga | 720 |
| ctacatgatg ataaagagta tggtcttagt gtcttcaagc caatgacaaa tgaatgtcta | 780 |
| aattggttaa accatcaacc aattagctca gtggtgtatg tatcatttgg aagtataacc | 840 |
| aaattaggag atgagcaaat ggaagaattg gcatggggtt tgaagaatag caacaagagc | 900 |
| ttcttgtggg ttgttaggtc tactgaagag cccaaacttc ccaacaactt tattgaggaa | 960 |
| ttaacaagtg aaaaaggctt agtggtgtca tggtgtccac aattacaagt gttggaacat | 1020 |
| gaatcgacag gttgttttct gacgcactgt ggatggaatt caactctgga agcgattagt | 1080 |
| ttgggagtgc caatggtggc aatgccacaa tggtctgatc aaccaacaaa tgcaaagctt | 1140 |
| gtgaaagatg tttgggaaat aggtgttaga gccaaacaag atgaaaaagg ggtagttaga | 1200 |
| agagaagtta tagaagaatg tataaagcta gtgatggaag aagataaagg aaaactaatt | 1260 |
| agagaaaatg caaagaaatg gaaggaaata gctagaaatg ttgtgaatga aggaggaagt | 1320 |
| tcagataaaa acattgaaga atttgtttcc aagttggtta ctatttccta a | 1371 |

SEQ ID NO: 211
Solanum lycopersicum

| | |
|---|---|
| MTTHKAHCLI LPFPGQGHIN PMLQFSKRLQ SKRVKITIAL TKSCLKTMQE LSTSVSIEAI | 60 |
| SDGYDDGGFH QAENFVAYIT RFKEVGSDTL SQLIKKLENS DCPVNCIVYD PFIPWAVEVA | 120 |
| KQFGLISAAF FTQNCVVDNL YYHVHKGVIK LPPTQNDEEI LIPGFPNSID ASDVPSFVIS | 180 |
| PEAERIVEML ANQFSNLDKV DYVLINSFYE LEKEVNEWMS KIYPIKTIGP TIPSMYLDKR | 240 |
| LHDDKEYGLS VFKPMTNECL NWLNHQPISS VVYVSFGSIT KLGDEQMEEL AWGLKNSNKS | 300 |
| FLWVVRSTEE PKLPNNFIEE LTSEKGLVVS WCPQLQVLEH ESTGCFLTHC GWNSTLEAIS | 360 |
| LGVPMVAMPQ WSDQPTNAKL VKDVWEIGVR AKQDEKGVVR REVIEECIKL VMEEDKGKLI | 420 |
| RENAKKWKEI ARNVVNEGGS SDKNIEEFVS KLVTIS | 456 |

SEQ ID NO: 212
Artificial Sequence

| | |
|---|---|
| atggctacca gtgactccat agttgacgac cgtaagcagc ttcatgttgc gacgttccca | 60 |
| tggcttgctt tcggtcacat cctcccttac cttcagcttt cgaaattgat agctgaaaag | 120 |
| ggtcacaaag tctcgtttct ttctaccacc agaaacattc aacgtctctc ttctcatatc | 180 |
| tcgccactca taaatgttgt tcaactcaca cttccacgtg tccaagagct gccggaggat | 240 |
| gcagaggcga ccactgacgt ccaccctgaa gatattccat atctcaagaa ggcttctgat | 300 |
| ggtcttcaac cggaggtcac ccggtttcta gaacaacact ctccggactg gattatttat | 360 |
| gattatactc actactggtt gccatccatc gcggctagcc tcggtatctc acgagcccac | 420 |
| ttctccgtca ccactccatg ggccattgct tatatgggac cctcagctga cgccatgata | 480 |
| aatggttcag atggtcgaac cacggttgag gatctcacga caccgcccaa gtggtttccc | 540 |
| tttccgacca aagtatgctg gcggaagcat gatcttgccc gactggtgcc ttacaaagct | 600 |
| ccggggatat ctgatggata ccgtatgggg atggttctta agggatctga ttgtttgctt | 660 |
| tccaaatgtt accatgagtt tggaactcaa tggctacctc ttttggagac actacaccaa | 720 |
| gtaccggtgg ttccggtggg attactgcca ccggaaatac ccggagacga gaaagatgaa | 780 |
| acatgggtgt caatcaagaa atggctcgat ggtaaacaaa aaggcagtgt ggtgtacgtt | 840 |
| gcattaggaa gcgaggcttt ggtgagccaa accgaggttg ttgagttagc attgggtctc | 900 |
| gagctttctg ggttgccatt tgtttgggct tatagaaaac caaaaggtcc cgcgaagtca | 960 |
| gactcggtgg agttgccaga cgggttcgtg gaacgaactc gtgaccgtgg gttggtctgg | 1020 |
| acgagttggg cacctcagtt acgaatactg agccatgagt cggtttgtgg tttcttgact | 1080 |
| cattgtggtt ctggatcaat tgtggaaggg ctaatgtttg gtcaccctct aatcatgcta | 1140 |
| ccgatttttg gggaccaacc tctgaatgct cgattactgg aggacaaaca ggtgggaatc | 1200 |
| gagataccaa gaaatgagga agatggttgc ttgaccaagg agtcggttgc tagatcactg | 1260 |
| aggtccgttg ttgtggaaaa agaaggggag atctacaagg cgaacgcgag ggagctgagt | 1320 |
| aaaatctata acgacactaa ggttgaaaaa gaatatgtaa gccaattcgt agactatttg | 1380 |
| gaaaagaatg cgcgtgcggt tgccatcgat catgagagtt aa | 1422 |

SEQ ID NO: 213
Stevia rebaudiana

| | |
|---|---|
| atggcggaac aacaaaagat caagaaatca ccacacgttc tactcatccc attcccttta | 60 |
| caaggccata taaacccttt catccagttt ggcaaacgat taatctccaa aggtgtcaaa | 120 |
| acaacacttg ttaccaccat ccacacctta aactcaaccc taaaccacag taacaccacc | 180 |
| accacctcca tcgaaatcca agcaatttcc gatggttgtg atgaaggcgg ttttatgagt | 240 |
| gcaggagaat catatttgga aacattcaaa caagttgggt ctaaatcact agctgactta | 300 |
| atcaagaagc ttcaaagtga aggaaccaca attgatgcaa tcatttatga ttctatgact | 360 |
| gaatgggttt tagatgttgc aattgagttt ggaatcgatg gtggttcgtt tttcactcaa | 420 |
| gcttgtgttg taaacagctt atattatcat gttcataagg gtttgatttc tttgccattg | 480 |
| ggtgaaactg tttcggttcc tggatttcca gtgcttcaac ggtgggagac accgttaatt | 540 |
| ttgcagaatc atgagcaaat acagagccct tggtctcaga tgttgtttgg tcagtttgct | 600 |
| aatattgatc aagcacgttg ggtcttcaca aatagttttt acaagctcga ggaagaggta | 660 |
| atagagtgga cgagaaagat atggaacttg aaggtaatcg ggccaacact tccatccatg | 720 |
| taccttgaca aacgacttga tgatgataaa gataacggat ttaatctcta caaagcaaac | 780 |
| catcatgagt gcatgaactg gttagacgat aagccaaagg aatcagttgt ttacgtagca | 840 |
| tttggtagcc tggtgaaaca tggacccgaa caagtggaag aaatcacacg ggctttaata | 900 |
| gatagtgatg tcaacttctt gtgggttatc aaacataaag aagagggaaa gctcccagaa | 960 |
| aatctttcgg aagtaataaa aaccggaaag ggtttgattg tagcatggtg caaacaattg | 1020 |
| gatgtgttag cacacgaatc agtaggatgc tttgttacac attgtgggtt caactcaact | 1080 |
| cttgaagcaa taagtcttgg agtccccgtt gttgcaatgc ctcaattttc ggatcaaact | 1140 |
| acaaatgcca agcttctaga tgaaattttg ggtgttggag ttagagttaa ggctgatgag | 1200 |
| aatgggatag tgagaagagg aaatcttgcg tcatgtatta agatgattat ggaggaggaa | 1260 |
| agaggagtaa taatccgaaa gaatgcggta aaatggaagg atttggctaa agtagccgtt | 1320 |
| catgaaggtg gtagctcaga caatgatatt gtcgaatttg taagtgagct aattaaggct | 1380 |
| taa | 1383 |

What is claimed is:

1. A recombinant host cell capable of producing one or more steviol glycosides and/or glycosylated steviol precursors, or a composition thereof, comprising:
(a) a gene encoding a polypeptide capable of glycosylating steviol or a steviol glycoside at its C-19 carboxyl position;
(b) a gene encoding a polypeptide capable of glycosylating steviol or a steviol glycoside at its C-13 hydroxyl position;
(c) a gene encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside; and/or
(d) a gene encoding a polypeptide capable of glycosylating a steviol precursor at its C-19 carboxyl or C-19 hydroxyl position;
wherein at least one of the genes is a recombinant gene.

2. The recombinant host cell of claim 1, wherein:
(a) the polypeptide capable of glycosylating steviol or a steviol glycoside at its C-19 carboxyl position is a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, and/or a UGT74F2-like UGT polypeptide;
(b) the polypeptide capable of glycosylating steviol or a steviol glycoside at its C-13 hydroxyl position is a UGT73C1 polypeptide, a UGT73C3 polypeptide, a UGT73C5 polypeptide, a UGT73C6 polypeptide, a UGT73C7 polypeptide, a UGT73E1 polypeptide, and/or a UGT76E12 polypeptide;
(c) the polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside is a UGT73C6 polypeptide, a CaUGT3 polypeptide, a UN32491 polypeptide, and/or a UN1671 polypeptide; and/or
(d) the polypeptide capable of glycosylating a steviol precursor at its C-19 carboxyl or C-19 hydroxyl position is a UGT73C1 polypeptide, a UGT73C3 polypeptide, a UGT73C5 polypeptide, a UGT73C6 polypeptide, a UGT73E1 polypeptide, a UGT74D1 polypeptide, a UGT75B1 polypeptide, a UGT75L6 polypeptide, a UGT76E12 polypeptide, a Olel polypeptide, a UGT5 polypeptide, a SA Gtase, a UDPG1 polypeptide, a UGT74F1 polypeptide, a UGT75D1 polypeptide, a UGT84B2 polypeptide, a CaUGT2 polypeptide, and/or a UGT74F2-like UGT polypeptide.

3. The recombinant host cell of claim 2, wherein:
the UGT73C1 polypeptide comprises a polypeptide having at least 60% identity to an amino acid sequence set forth in SEQ ID NO:127, the UGT73C3 polypeptide comprises a polypeptide having at least 60% identity to an amino acid sequence set forth in SEQ ID NO:133, the UGT73C5 polypeptide comprises a polypeptide having at least 60% identity to an amino acid sequence set forth in SEQ ID NO:135, the UGT73C6 polypeptide comprises a polypeptide having at least 60% identity to an amino acid sequence set forth in SEQ ID NO:137, the UGT73E1 polypeptide comprises a polypeptide having at least 50% identity to an amino acid sequence set forth in SEQ ID NO:141, the UGT74D1 polypeptide comprises a polypeptide having at least 50% identity to an amino acid sequence set forth in SEQ ID NO:143, the UGT75B1 polypeptide comprises a polypeptide having at least 50% sequence identity to an amino acid sequence set forth in SEQ ID NO:145, the UGT75L6 polypeptide comprises a polypeptide having at least 60% sequence identity to an amino acid sequence set forth in SEQ ID NO:147, the UGT76E12 polypeptide comprises a polypeptide having at least 60% sequence identity to an amino acid sequence set forth in SEQ ID NO:153, the Olel polypeptide comprises a polypeptide having at least 55% identity to an amino acid sequence set forth in SEQ ID NO:177, the UGT5 polypeptide comprises a polypeptide having at least 65% identity to an amino acid sequence set forth in SEQ ID NO:181, the SA Gtase polypeptide comprises a polypeptide having at least 55% identity to an amino acid sequence set forth in SEQ ID NO:183, the UDPG1 polypeptide comprises a polypeptide having at least 50% sequence identity to an amino acid sequence set forth in SEQ ID NO:185, the UN1671 polypeptide comprises a polypeptide having at least 45% identity to an amino acid sequence set forth in SEQ ID NO:201, the UGT74F1 polypeptide comprises a polypeptide having at least 50% sequence identity to an amino acid sequence set forth in SEQ ID NO:203, the UGT75D1 polypeptide comprises a polypeptide having at least 50% sequence identity to an amino acid sequence set forth in SEQ ID NO:205, the UGT84B2 polypeptide comprises a polypeptide having at least 40% sequence identity to an amino acid sequence set forth in SEQ ID NO:207, the UGT74F2-like UGT polypeptide comprises a polypeptide having at least 55% identity to an amino acid sequence set forth in SEQ ID NO:211, the UGT73C7 polypeptide comprises a polypeptide having at least 60% identity to an amino acid sequence set forth in SEQ ID NO:139, the CaUGT3 polypeptide comprises a polypeptide having at least 50% identity to an amino acid sequence set forth in SEQ ID NO:169, the UN32491 polypeptide comprises a polypeptide having at least 50% identity to an amino acid sequence set forth in SEQ ID NO:199, and/or the CaUGT2 polypeptide comprises a polypeptide having at least 55% identity to an amino acid sequence set forth in SEQ ID NO:209.

4. The recombinant host cell of any one of claims 1-3, wherein the recombinant host cell further comprises:
(a) a gene encoding a polypeptide capable of synthesizing geranylgeranyl pyrophosphate (GGPP) from farnesyl diphosphate (FPP) and isopentenyl diphosphate (IPP);
(b) a gene encoding a polypeptide capable of synthesizing ent-copalyl diphosphate from GGPP;
(c) a gene encoding an a polypeptide capable of synthesizing ent-kaurene from ent-copalyl diphosphate;
(d) a gene encoding a polypeptide capable of synthesizing ent-kaurenoic acid from ent-kaurene;
(e) a gene encoding a polypeptide capable of reducing cytochrome P450 complex;
(f) a gene encoding a polypeptide capable of synthesizing steviol from ent-kaurenoic acid;
(g) a gene encoding a polypeptide capable of glycosylating steviol or a steviol glycoside at its C-13 hydroxyl position thereof;
(h) a gene encoding a polypeptide capable of beta 1,3 glycosylation of the C3′ of the 13-O-glucose, 19-O-glucose, or both 13-O-glucose and 19-O-glucose of a steviol glycoside;
(i) a gene encoding a polypeptide capable of glycosylating steviol or a steviol glycoside at its C-19 carboxyl position; and/or
(k) a gene encoding a polypeptide capable of beta 1,2 glycosylation of the C2′ of the 13-O-glucose, 19-O-glucose, or both 13-O-glucose and 19-O-glucose of a steviol glycoside;
wherein at least one of the genes is a recombinant gene.

5. The recombinant host cell of claim 4, wherein:
(a) the polypeptide capable of synthesizing GGPP comprises a polypeptide having at least 70% sequence identity to the amino acid sequence set forth in SEQ ID NO:20, SEQ ID NO:22, SEQ ID NO:24, SEQ ID NO:26, SEQ ID NO:28, SEQ ID NO:30, SEQ ID NO:32, or SEQ ID NO:116;
(b) the polypeptide capable of synthesizing ent-copalyl diphosphate comprises a polypeptide having at least 70% sequence identity to the amino acid sequence set forth in SEQ ID NO:34, SEQ ID NO:36, SEQ ID NO:38, SEQ ID NO:40, SEQ ID NO:42, or SEQ ID NO:120;
(c) the polypeptide capable of synthesizing ent-kaurene comprises a polypeptide having at least 70% sequence identity to the amino acid sequence set forth in SEQ ID NO:44, SEQ ID NO:46, SEQ ID NO:48, SEQ ID NO:50, or SEQ ID NO:52;
(d) the polypeptide capable of synthesizing ent-kaurenoic acid comprises a polypeptide having at least 70% sequence identity to the amino acid sequence set forth in SEQ ID NO:60, SEQ ID NO:62, SEQ ID NO:117, SEQ ID NO:66, SEQ ID NO:68, SEQ ID NO:70, SEQ ID NO:72, SEQ ID NO:74, or SEQ ID NO:76;
(e) the polypeptide capable of reducing cytochrome P450 complex comprises a polypeptide having at least 70% sequence identity to the amino acid sequence set forth in SEQ ID NO:78, SEQ ID NO:80, SEQ ID NO:82, SEQ ID NO:84, SEQ ID NO:86, SEQ ID NO:88, SEQ ID NO:90, SEQ ID NO:92;
(f) the polypeptide capable of synthesizing steviol comprises a polypeptide having at least 70% sequence identity to the amino acid sequence set forth in SEQ ID NO:94, SEQ ID NO:97, SEQ ID NO:100, SEQ ID NO:101, SEQ ID NO:102, SEQ ID NO:103, SEQ ID NO:104, SEQ ID NO:106, SEQ ID NO:108, SEQ ID NO:110, SEQ ID NO:112, or SEQ ID NO:114;
(g) the polypeptide capable of glycosylating steviol or a steviol glycoside at its C-13 hydroxyl position thereof comprises a polypeptide having at least 55% sequence identity to the amino acid sequence set forth in SEQ ID NO:7;
(h) the polypeptide capable of beta 1,3 glycosylation of the C3′ of the 13-O-glucose, 19-O-glucose, or both 13-O-glucose and 19-O-glucose of a steviol glycoside comprises a polypeptide having at least 50% sequence identity to the amino acid sequence set forth in SEQ ID NO:9;
(i) the polypeptide capable of glycosylating steviol or a steviol glycoside at its C-19 carboxyl position comprises a polypeptide having at least 55% sequence identity to the amino acid sequence set forth in SEQ ID NO:4; and/or
(k) the polypeptide capable of beta 1,2 glycosylation of the C2′ of the 13-O-glucose, 19-O-glucose, or both 13-O-glucose and 19-O-glucose of a steviol glycoside comprises a polypeptide having 80% or greater identity to the amino acid sequence set forth in SEQ ID NO:11; a polypeptide having 80% or greater identity to the amino acid sequence set forth in SEQ ID NO:13; or a polypeptide having at least 65% sequence identity to the amino acid sequence set forth in SEQ ID NO:16.

6. The recombinant host cell of any of claims 1-5, wherein expression of the one or more recombinant genes increases an amount of the one or more steviol glycosides and/or glycosylated steviol precursors, or a composition thereof accumulated by the cell relative to a corresponding host lacking the one or more recombinant genes.

7. The recombinant host cell of claim 6, wherein expression of the one or more recombinant genes increases the amount of the one or more steviol glycosides and/or glycosylated steviol precursors, or the composition thereof, accumulated by the cell by at least about 5%, at least about 10%, at least about 25%, at least about 50%, at least about 75%, or at least about 100% relative to a corresponding host lacking the one or more recombinant genes.

8. The recombinant host cell of claim 6 or 7, wherein expression of the one or more recombinant genes increases the amount of ent-kaurenoic acid+2Glc (#7), ent-kaurenoic acid+3Glc (isomer 1), ent-kaurenoic acid+3Glc (isomer 2), steviol-13-O-glucoside (13-SMG), Rebaudioside A (RebA), Rebaudioside B (RebB), Steviol+4Glc (#36), Steviol+6Glc (isomer 1), Steviol+7Glc (isomer 2), and/or ent-Kaurenol+3Glc (isomer 1 and/or isomer 2) accumulated by the cell relative to a corresponding host lacking the one or more recombinant genes.

9. The recombinant host cell of any one of claims 1-8, wherein the one or more steviol glycosides and/or glycosylated steviol precursors are, or the composition thereof comprises, steviol-13-O-glucoside (13-SMG), steviol-19-O-glucoside (19-SMG), steviol-1,2-bioside, steviol-1,3-bioside, 1,2-stevioside, 1,3-stevioside, rubusoside, Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, a mono-glycosylated ent-kaurenoic acid, a di-glycosylated ent-kaurenoic acid, a tri-glycosylated ent-kaurenoic acid, a mono-glycosylated ent-kaurenols, a di-glycosylated ent-kaurenol, a tri-glycosylated ent-kaurenol, a tri-glycosylated steviol glycoside, a tetra-glycosylated steviol glycoside, a penta-glycosylated steviol glycoside, a hexa-glycosylated steviol glycoside, a hepta-glycosylated steviol glycoside, or an isomer thereof.

10. The recombinant host cell of claim 9, wherein the mono-glycosylated ent-kaurenoic acid comprises KA1.58 of Table 1 and/or the penta-glycosylated steviol comprises Compound 5.24 of Table 1.

11. The recombinant host cell of claim 1-10, wherein the recombinant host cell comprises a plant cell, a mammalian cell, an insect cell, a fungal cell, an algal cell, or a bacterial cell.

12. A method of producing in a cell culture one or more steviol glycosides and/or glycosylated steviol precursors, or a composition thereof, comprising growing the recombinant host cell of any one of claims 1-11 in the cell culture, under conditions in which the genes are expressed, and wherein the one or more steviol glycosides and/or glycosylated steviol precursors, or the composition thereof is produced by the recombinant host cell.

13. The method of claim 12, wherein the genes are constitutively expressed and/or expression of the genes is induced.

14. The method of claim 12 or 13, wherein an amount of ent-kaurenoic acid+2Glc (#7), ent-kaurenoic acid+3Glc (isomer 1), ent-kaurenoic acid+3Glc (isomer 2), 13-SMG, RebA, RebB, Steviol+4Glc (#36), Steviol+6Glc (isomer 1), Steviol+7Glc (isomer 2), and/or ent-Kaurenol+3Glc (isomer 1 and/or isomer 2) accumulated by the recombinant host cell is increased by at least about 5% relative to a corresponding host lacking the one or more recombinant genes.

15. The method of any one of claims 12-14, further comprising isolating from the cell cultures the one or more steviol glycosides and/or glycosylated steviol precursors or the composition thereof produced thereby.

16. The method of claim 15, wherein the isolating step comprises:
(a) providing the cell culture comprising the one or more steviol glycosides and/or glycosylated steviol precursors, or the composition thereof;
(b) separating a liquid phase of the cell culture from a solid phase of the cell culture to obtain a supernatant comprising the produced one or more steviol glycosides and/or glycosylated steviol precursors, or the composition thereof;
(c) providing one or more adsorbent resins, comprising providing the adsorbent resins in a packed column; and
(d) contacting the supernatant of step (b) with the one or more adsorbent resins in order to obtain at least a portion of the produced one or more steviol glycosides and/or glycosylated steviol precursors, or the composition thereof, thereby isolating the produced one or more steviol glycosides or the steviol glycoside composition;
or
(a) providing the cell culture comprising the one or more steviol glycosides and/or glycosylated steviol precursors, or the composition thereof;
(b) separating a liquid phase of the cell culture from a solid phase of the cell culture to obtain a supernatant comprising the produced one or more steviol glycosides and/or glycosylated steviol precursors, or the composition thereof;
(c) providing one or more ion exchange or ion exchange or reversed-phase chromatography columns; and
(d) contacting the supernatant of step (b) with the one or more ion exchange or ion exchange or reversed-phase chromatography columns in order to obtain at least a portion of the produced one or more steviol glycosides and/or glycosylated steviol precursors, or the composition thereof, thereby isolating the produced one or more steviol glycosides and/or glycosylated steviol precursors, or the composition thereof;
or
(a) providing the cell culture comprising the one or more steviol glycosides and/or glycosylated steviol precursors, or the composition thereof;
(b) separating a liquid phase of the cell culture from a solid phase of the cell culture to obtain a supernatant comprising the produced one or more steviol glycosides and/or glycosylated steviol precursors, or the composition thereof;
(c) crystallizing or extracting the produced one or more steviol glycosides and/or glycosylated steviol precursors, or the composition thereof, thereby isolating the produced one or more steviol glycosides and/or glycosylated steviol precursors, or the composition thereof.

17. The method of any one of claims 12-14, further comprising recovering from the cell culture the one or more steviol glycosides and/or glycosylated steviol precursors or the composition thereof from the cell culture, wherein the cell culture is enriched for the one or more steviol glycosides and/or glycosides of a steviol precursor, or the composition thereof relative to a steviol glycoside composition from a Stevia plant and has a reduced level of Stevia plant-derived components relative to a plant-derived Stevia extract.

18. The method of claim 17, wherein the recovered one or more steviol glycosides and/or glycosylated steviol precursors, or the composition thereof are present in relative amounts that are different from a steviol glycoside composition recovered from a Stevia plant and have a reduced level of Stevia plant-derived components relative to a plant-derived Stevia extract.

19. A method for producing one or more steviol glycosides and/or glycosylated steviol precursors, or the composition thereof, comprising whole cell bioconversion of plant-derived or synthetic steviol, steviol precursors and/or steviol glycosides in a cell culture medium of a recombinant host cell using:
(a) a gene encoding a polypeptide capable of glycosylating steviol or a steviol glycoside at its C-19 carboxyl position;
(b) a gene encoding a polypeptide capable of glycosylating steviol or a steviol glycoside at its C-13 hydroxyl position;
(c) a gene encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside; and/or
(d) a gene encoding a polypeptide capable of glycosylating a steviol precursor at its C-19 carboxyl or C-19 hydroxyl position;
wherein at least one of the polypeptides is a recombinant polypeptide expressed in the recombinant host cell; and producing the one or more steviol glycosides and/or glycosylated steviol precursors, or the composition thereof, thereby.

20. The method of claim 19, wherein:
(a) the polypeptide capable of glycosylating steviol or a steviol glycoside at its C-19 carboxyl position is a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a Olel polypeptide, a UGT5 polypeptide, a SA Gtase polypeptide, a UDPG1 polypeptide, a UN1671 polypeptide, a UGT74F1 polypeptide, a UGT84B2 polypeptide, and/or a UGT74F2-like UGT polypeptide;
(b) the polypeptide capable of glycosylating steviol or a steviol glycoside at its C-13 hydroxyl position is a UGT73C1 polypeptide, a UGT73C3 polypeptide, a UGT73C5 polypeptide, a UGT73C6 polypeptide, a UGT73C7 polypeptide, a UGT73E1 polypeptide, and/or a UGT76E12 polypeptide;
(c) the polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside is a UGT73C6 polypeptide, a CaUGT3 polypeptide, a UN32491 polypeptide, and/or a UN1671 polypeptide; and/or
(d) the polypeptide capable of glycosylating a steviol precursor at its C-19 carboxyl or C-19 hydroxyl position is a UGT73C1 polypeptide, a UGT73C3 polypeptide, a UGT73C5 polypeptide, a UGT73C6 polypeptide, a UGT73E1 polypeptide, a UGT75B1 polypeptide, a UGT75L6 polypeptide, a UGT76E12 polypeptide, a Olel polypeptide, a UGT5 polypeptide, a SA Gtase, a UDPG1 polypeptide, a UGT74F1 polypeptide, a UGT75D1 polypeptide, a UGT84B2 polypeptide, and/or a UGT74F2-like UGT polypeptide.

21. The method of claim 20, wherein:
the UGT73C1 polypeptide comprises a polypeptide having at least 60% identity to an amino acid sequence set forth in SEQ ID NO:127, the UGT73C3 polypeptide comprises a polypeptide having at least 60% identity to an amino acid sequence set forth in SEQ ID NO:133, the UGT73C5 polypeptide comprises a polypeptide having at least 60% identity to an amino acid sequence set forth in SEQ ID NO:135, the UGT73C6 polypeptide comprises a polypeptide having at least 60% identity to an amino acid sequence set forth in SEQ ID NO:137, the UGT73E1 polypeptide comprises a polypeptide having at least 50% identity to an amino acid sequence set forth in SEQ ID NO:141, a UGT74D1 polypeptide comprises a polypeptide having at least 50% identity to an amino acid sequence set forth in SEQ ID NO:143, the UGT75B1 polypeptide comprises a polypeptide having at least 50% sequence identity to an amino acid sequence set forth in SEQ ID NO:145, the UGT75L6 polypeptide comprises a polypeptide having at least 60% sequence identity to an amino acid sequence set forth in SEQ ID NO:147, the UGT76E12 polypeptide comprises a polypeptide having at least 60% sequence identity to an amino acid sequence set forth in SEQ ID NO:153, the Olel polypeptide comprises a polypeptide having at least 55% identity to an amino acid sequence set forth in SEQ ID NO:177, the UGT5 polypeptide comprises a polypeptide having at least 65% identity to an amino acid sequence set forth in SEQ ID NO:181, the SA Gtase polypeptide comprises a polypeptide having at least 55% identity to an amino acid sequence set forth in SEQ ID NO:183, the UDPG1 polypeptide comprises a polypeptide having at least 50% sequence identity to an amino acid sequence set forth in SEQ ID NO:185, the UN1671 polypeptide comprises a polypeptide having at least 45% identity to an amino acid sequence set forth in SEQ ID NO:201, the UGT74F1 polypeptide comprises a polypeptide having at least 50% sequence identity to an amino acid sequence set forth in SEQ ID NO:203, the UGT75D1 polypeptide comprises a polypeptide having at least 50% sequence identity to an amino acid sequence set forth in SEQ ID NO:205, the UGT84B2 polypeptide comprises a polypeptide having at least 40% sequence identity to an amino acid sequence set forth in SEQ ID NO:207, the UGT74F2-like UGT polypeptide comprises a polypeptide having at least 55% identity to an amino acid sequence set forth in SEQ ID NO:211, the UGT73C7 polypeptide comprises a polypeptide having at least 60% identity to an amino acid sequence set forth in SEQ ID NO:139, the CaUGT3 polypeptide comprises a polypeptide having at least 50% identity to an amino acid sequence set forth in SEQ ID NO:169, the UN32491 polypeptide comprises a polypeptide having at least 50% identity to an amino acid sequence set forth in SEQ ID NO:199, or the CaUGT2 polypeptide comprises a polypeptide having at least 55% identity to an amino acid sequence set forth in SEQ ID NO:209.

22. The method of any one of claims 12-21, wherein the recombinant host cell is a plant cell, a mammalian cell, an insect cell, a fungal cell, an algal cell or a bacterial cell.

23. An in vitro method for producing one or more steviol glycosides and/or glycosylated steviol precursors, or a composition thereof comprising adding:
(a) a UGT85C2 polypeptide having at least 55% identity to an amino acid sequence set forth in SEQ ID NO:7;
(b) a UGT76G1 polypeptide having at least 50% identity to an amino acid sequence set forth in SEQ ID NO:9;
(c) a UGT74G1 polypeptide having at least 55% identity to an amino acid sequence set forth in SEQ ID NO:4;
(d) a UGT91D2 functional homolog polypeptide comprising a UGT91D2e polypeptide having 90% or greater identity to an amino acid sequence set forth in SEQ ID NO:11 or a UGT91D2e-b polypeptide having 90% or greater identity to an amino acid sequence set forth in SEQ ID NO:13;
(e) a EUGT11 polypeptide having at least 65% identity to an amino acid sequence set forth in SEQ ID NO:16; and/or
(f) a UGT73C1 polypeptide comprises a polypeptide having at least 60% identity to an amino acid sequence set forth in SEQ ID NO:127, a UGT73C3 polypeptide comprises a polypeptide having at least 60% identity to an amino acid sequence set forth in SEQ ID NO:133, a UGT73C5 polypeptide comprises a polypeptide having at least 60% identity to an amino acid sequence set forth in SEQ ID NO:135, a UGT73C6 polypeptide comprises a polypeptide having at least 60% identity to an amino acid sequence set forth in SEQ ID NO:137, a UGT73E1 polypeptide comprises a polypeptide having at least 50% identity to an amino acid sequence set forth in SEQ ID NO:141, a UGT74D1 polypeptide comprises a polypeptide having at least 50% identity to an amino acid sequence set forth in SEQ ID NO:143, a UGT75B1 polypeptide comprises a polypeptide having at least 50% sequence identity to an amino acid sequence set forth in SEQ ID NO:145, a UGT75L6 polypeptide comprises a polypeptide having at least 60% sequence identity to an amino acid sequence set forth in SEQ ID NO:147, a UGT76E12 polypeptide comprises a polypeptide having at least 60% sequence identity to an amino acid sequence set forth in SEQ ID NO:153, a Olel polypeptide comprises a polypeptide having at least 55% identity to an amino acid sequence set forth in SEQ ID NO:177, a UGTS polypeptide comprises a polypeptide having at least 65% identity to an amino acid sequence set forth in SEQ ID NO:181, a SA Gtase polypeptide comprises a polypeptide having at least 55% identity to an amino acid sequence set forth in SEQ ID NO:183, a UDPG1 polypeptide comprises a polypeptide having at least 50% sequence identity to an amino acid sequence set forth in SEQ ID NO:185, a UN1671 polypeptide comprises a polypeptide having at least 45% identity to an amino acid sequence set forth in SEQ ID NO:201, a UGT74F1 polypeptide comprises a polypeptide having at least 50% sequence identity to an amino acid sequence set forth in SEQ ID NO:203, a UGT75D1 polypeptide comprises a polypeptide having at least 50% sequence identity to an amino acid sequence set forth in SEQ ID NO:205, a UGT84B2 polypeptide comprises a polypeptide having at least 40% sequence identity to an amino acid sequence set forth in SEQ ID NO:207, a UGT74F2-like UGT polypeptide comprises a polypeptide having at least 55% identity to an amino acid sequence set forth in SEQ ID NO:211, a UGT73C7 polypeptide comprises a polypeptide having at least 60% identity to an amino acid sequence set forth in SEQ ID NO:139, a CaUGT3 polypeptide comprises a polypeptide having at least 50% identity to an amino acid sequence set forth in SEQ ID NO:169, a UN32491 polypeptide comprises a polypeptide having at least 50% identity to an amino acid sequence set forth in SEQ ID NO:199, or a CaUGT2 polypeptide comprises a polypeptide having at least 55% identity to an amino acid sequence set forth in SEQ ID NO:209;
and a plant-derived or synthetic steviol glycoside precursor or a plant-derived or synthetic steviol precursor to a reaction mixture;
wherein at least one of the polypeptides is a recombinant polypeptide; and
producing the one or more steviol glycosides and/or glycosylated steviol precursors, or the composition thereof, thereby.

24. The method of claim 23, wherein the reaction mixture comprises:
(a) glucose, fructose, sucrose, xylose, rhamnose, uridine diphosphate (UDP)-glucose, UDP-rhamnose, UDP-xylose, and/or N-acetyl-glucosamine; and/or
(b) reaction buffer and/or salts.

25. The method of any one of claims 12-24, wherein the one or more steviol glycosides and/or glycosylated steviol precursors are, or the composition thereof comprises, 13-SMG, 19-SMG, steviol-1,2-bioside, steviol-1,3-bioside, 1,2-stevioside, 1,3-stevioside, rubusoside, RebA, RebB, RebC, RebD, RebE, RebF, RebM, RebQ, RebI, dulcoside A, a mono-glycosylated ent-kaurenoic acid, a di-glycosylated ent-kaurenoic acid, a tri-glycosylated ent-kaurenoic acid, a mono-glycosylated ent-kaurenols, a di-glycosylated ent-kaurenol, a tri-glycosylated ent-kaurenol, a tri-glycosylated steviol glycoside, a tetra-glycosylated steviol glycoside, a penta-glycosylated steviol glycoside, a hexa-glycosylated steviol glycoside, a hepta-glycosylated steviol glycoside, or an isomer thereof.

26. The method of claim 25, wherein the mono-glycosylated ent-kaurenoic acid comprises KA1.58 of Table 1 and/or the penta-glycosylated steviol comprises Compound 5.24 of Table 1.

27. A cell culture, comprising the recombinant host cell of any one of claims 1-11, the cell culture further comprising:
(a) one or more steviol glycosides and/or glycosylated steviol precursors, or the composition thereof produced by the recombinant host cell,
(b) glucose, fructose, sucrose, xylose, rhamnose, UDP-glucose, UDP-rhamnose, UDP-xylose, and/or N-acetyl-glucosamine; and
(c) supplemental nutrients comprising trace metals, vitamins, salts, yeast nitrogen base (YNB), and/or amino acids;
wherein the one or more steviol glycosides and/or glycosylated steviol precursors, or the composition thereof is present at a concentration of at least 1 mg/liter of the cell culture;
wherein the cell culture is enriched for the one or more steviol glycosides and/or glycosides of a steviol precursor, or the composition thereof relative to a steviol glycoside composition from a Stevia plant and has a reduced level of Stevia plant-derived components relative to a plant-derived Stevia extract.

28. A cell lysate from the recombinant host cell of any one of claims 1-11 grown in the cell culture, comprising:
(a) one or more steviol glycosides and/or glycosylated steviol precursors, or the composition thereof produced by the recombinant host cell;
(b) glucose, fructose, sucrose, xylose, rhamnose, UDP-glucose, UDP-rhamnose, UDP-xylose, and/or N-acetyl-glucosamine; and/or
(c) supplemental nutrients comprising trace metals, vitamins, salts, yeast nitrogen base, YNB, and/or amino acids;
wherein the one or more steviol glycosides and/or glycosylated steviol precursors, or the composition thereof produced by the recombinant host cell is present at a concentration of at least 1 mg/liter of the cell culture.

29. A reaction mixture, comprising:
(a) a UGT85C2 polypeptide having at least 55% identity to an amino acid sequence set forth in SEQ ID NO:7;
(b) a UGT76G1 polypeptide having at least 50% identity to an amino acid sequence set forth in SEQ ID NO:9;
(c) a UGT74G1 polypeptide having at least 55% identity to an amino acid sequence set forth in SEQ ID NO:4;
(d) a UGT91D2 functional homolog polypeptide comprising a UGT91D2e polypeptide having 90% or greater identity to an amino acid sequence set forth in SEQ ID NO:11 or a UGT91D2e-b polypeptide having 90% or greater identity to an amino acid sequence set forth in SEQ ID NO:13;
(e) a EUGT11 polypeptide having at least 65% identity to an amino acid sequence set forth in SEQ ID NO:16; and/or
(f) a UGT73C1 polypeptide comprises a polypeptide having at least 60% identity to an amino acid sequence set forth in SEQ ID NO:127, a UGT73C3 polypeptide comprises a polypeptide having at least 60% identity to an amino acid sequence set forth in SEQ ID NO:133, a UGT73C5 polypeptide comprises a polypeptide having at least 60% identity to an amino acid sequence set forth in SEQ ID NO:135, a UGT73C6 polypeptide comprises a polypeptide having at least 60% identity to an amino acid sequence set forth in SEQ ID NO:137, a UGT73E1 polypeptide comprises a polypeptide having at least 50% identity to an amino acid sequence set forth in SEQ ID NO:141, a UGT75B1 polypeptide comprises a polypeptide having at least 50% sequence identity to an amino acid sequence set forth in SEQ ID NO:145, a UGT75L6 polypeptide comprises a polypeptide having at least 60% sequence identity to an amino acid sequence set forth in SEQ ID NO:147, a UGT76E12 polypeptide comprises a polypeptide having at least 60% sequence identity to an amino acid sequence set forth in SEQ ID NO:153, a Olel polypeptide comprises a polypeptide having at least 55% identity to an amino acid sequence set forth in SEQ ID NO:177, a UGT5 polypeptide comprises a polypeptide having at least 65% identity to an amino acid sequence set forth in SEQ ID NO:181, a SA Gtase polypeptide comprises a polypeptide having at least 55% identity to an amino acid sequence set forth in SEQ ID NO:183, a UDPG1 polypeptide comprises a polypeptide having at least 50% sequence identity to an amino acid sequence set forth in SEQ ID NO:185, a UN1671 polypeptide comprises a polypeptide having at least 45% identity to an amino acid sequence set forth in SEQ ID NO:201, a UGT74F1 polypeptide comprises a polypeptide having at least 50% sequence identity to an amino acid sequence set forth in SEQ ID NO:203, a UGT75D1 polypeptide comprises a polypeptide having at least 50% sequence identity to an amino acid sequence set forth in SEQ ID NO:205, a UGT84B2 polypeptide comprises a polypeptide having at least 40% sequence identity to an amino acid sequence set forth in SEQ ID NO:207, a UGT74F2-like UGT polypeptide comprises a polypeptide having at least 55% identity to an amino acid sequence set forth in SEQ ID NO:211, a UGT73C7 polypeptide comprises a polypeptide having at least 60% identity to an amino acid sequence set forth in SEQ ID NO:139, a CaUGT3 polypeptide comprises a polypeptide having at least 50% identity to an amino acid sequence set forth in SEQ ID NO:169, or a UN32491 polypeptide comprises a polypeptide having at least 50% identity to an amino acid sequence set forth in SEQ ID NO:199;
and further comprising:
(g) one or more steviol glycosides and/or glycosylated steviol precursors, or a composition thereof;
(h) glucose, fructose, sucrose, xylose, rhamnose, uridine diphosphate (UDP)-glucose, UDP-rhamnose, UDP-xylose, and/or N-acetyl-glucosamine; and/or
(i) reaction buffer and/or salts.

30. A composition of one or more steviol glycosides and/or glycosylated steviol precursors produced by the recombinant host cell of any one of claims 1-11;
wherein the one or more steviol glycosides and/or glycosylated steviol precursors produced by the recombinant host cell are present in relative amounts that are different from a steviol glycoside composition from a Stevia plant and have a reduced level of Stevia plant-derived components relative to a plant-derived Stevia extract.

31. A composition of one or more steviol glycosides and/or glycosylated steviol precursors produced by the method of any one of claims 12-26;
wherein the one or more steviol glycosides and/or glycosylated steviol precursors produced by the recombinant host cell are present in relative amounts that are different from a steviol glycoside composition from a Stevia plant and have a reduced level of Stevia plant-derived components relative to a plant-derived Stevia extract.

32. A sweetener composition, comprising one or more steviol glycosides and/or glycosylated steviol precursors of claim 30 or 31.

33. A food product, comprising the sweetener composition of claim 32.

34. A beverage or a beverage concentrate, comprising the sweetener composition of claim 32.

35. An isolated nucleic acid molecule encoding a polypeptide capable of glycosylating steviol or a steviol glycoside at its C-19 carboxyl position or a catalytically active portion thereof, wherein the encoded polypeptide capable of glycosylating steviol or a steviol glycoside at its C-19 carboxyl position or the catalytically active portion thereof has at least 60% sequence identity to the amino acid sequence set forth in SEQ ID NO:127, at least 60% sequence identity to the amino acid sequence set forth in SEQ ID NO:133, at least 60% sequence identity to the amino acid sequence set forth in SEQ ID NO:135, at least 60% sequence identity to the amino acid sequence set forth in SEQ ID NO:137, at least 50% sequence identity to the amino acid sequence set forth in SEQ ID NO:141, at least 50% sequence identity to the amino acid sequence set forth in SEQ ID NO:145, at least 60% sequence identity to the amino acid sequence set forth in SEQ ID NO:147, at least 55% sequence identity to the amino acid sequence set forth in SEQ ID NO:177, at least 65% sequence identity to the amino acid sequence set forth in SEQ ID NO:181, at least 55% sequence identity to the amino acid sequence set forth in SEQ ID NO:183, at least 50% sequence identity to the amino acid sequence set forth in SEQ ID NO:185, at least 45% sequence identity to the amino acid sequence set forth in SEQ ID NO:201, at least 50% sequence identity to the amino acid sequence set forth in SEQ ID NO:203, at least 40% sequence identity to the amino acid sequence set forth in SEQ ID NO:207, or at least 55% sequence identity to the amino acid sequence set forth in SEQ ID NO:211.

36. An isolated nucleic acid molecule encoding a polypeptide capable of glycosylating steviol or a steviol glycoside at its C-13 hydroxyl position or a catalytically active portion thereof, wherein the encoded polypeptide capable of glycosylating steviol or a steviol glycoside at its C-13 hydroxyl position or the catalytically active portion thereof has at least 60% sequence identity to the amino acid sequence set forth in SEQ ID NO:127, at least 60% sequence identity to the amino acid sequence set forth in SEQ ID NO:133, at least 60% sequence identity to the amino acid sequence set forth in SEQ ID NO:135, at least 60% sequence identity to the amino acid sequence set forth in SEQ ID NO:137, at least 60% sequence identity to the amino acid sequence set forth in SEQ ID NO:139, at least 50% sequence identity to the amino acid sequence set forth in SEQ ID NO:141, or at least 60% sequence identity to the amino acid sequence set forth in SEQ ID NO:153.

37. An isolated nucleic acid molecule encoding a polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside or a catalytically active portion thereof, wherein the encoded polypeptide capable of beta-1,2-glycosylation of the C2′ and/or beta-1,3-glycosylation of the C3′ of the 13-O-glucose, 19-O-glucose, or both 13-O-glucose and 19-O-glucose of a steviol glycoside or the catalytically active portion thereof has at least 60% sequence identity to the amino acid sequence set forth in SEQ ID NO:137, at least 50% sequence identity to the amino acid sequence set forth in SEQ ID NO:169, at least 50% sequence identity to the amino acid sequence set forth in SEQ ID NO:199, or at least 45% sequence identity to the amino acid sequence set forth in SEQ ID NO:201.

38. An isolated nucleic acid molecule encoding a polypeptide capable of glycosylating a steviol precursor at its C-19 carboxyl or C-19 hydroxyl position or a catalytically active portion thereof, wherein the encoded polypeptide capable of glycosylating a steviol precursor at its C-19 carboxyl or C-19 hydroxyl position or the catalytically active portion thereof has at least 60% sequence identity to the amino acid sequence set forth in SEQ ID NO:127, at least 60% sequence identity to the amino acid sequence set forth in SEQ ID NO:133, at least 60% sequence identity to the amino acid sequence set forth in SEQ ID NO:135, at least 60% sequence identity to the amino acid sequence set forth in SEQ ID NO:137, at least 50% sequence identity to the amino acid sequence set forth in SEQ ID NO:141, at least 50% sequence identity to the amino acid sequence set forth in SEQ ID NO:145, at least 60% sequence identity to the amino acid sequence set forth in SEQ ID NO:147, at least 60% sequence identity to the amino acid sequence set forth in SEQ ID NO:153, at least 55% sequence identity to the amino acid sequence set forth in SEQ ID NO:177, at least 65% sequence identity to the amino acid sequence set forth in SEQ ID NO:181, at least 55% sequence identity to the amino acid sequence set forth in SEQ ID NO:183, at least 50% sequence identity to the amino acid sequence set forth in SEQ ID NO:185, at least 50% sequence identity to the amino acid sequence set forth in SEQ ID NO:203, at least 50% sequence identity to the amino acid sequence set forth in SEQ ID NO:205, at least 40% sequence identity to the amino acid sequence set forth in SEQ ID NO:207, or at least 55% sequence identity to the amino acid sequence set forth in SEQ ID NO:211.

39. The isolated nucleic acid of any one of claims 35-38, wherein the nucleic acid is cDNA.


SEQUENCE LISTING (213 sequences)

SEQ ID NO: 1-2 - skipped (2 entries)

SEQ ID NO: 3 - DNA, 1383 bp, Artificial Sequence
  Codon-optimized UGT74G1
atggcagagcaacaaaagatcaaaaagtcacctcacgtcttacttattccatttcctctgcaaggacatatcaacccattcatacaatttgggaaaagattgattagtaagggtgtaaagacaacactggtaaccactatccacactttgaattctactctgaaccactcaaatactactactacaagtatagaaattcaagctatatcagacggatgcgatgagggtggctttatgtctgccggtgaatcttacttggaaacattcaagcaagtgggatccaagtctctggccgatctaatcaaaaagttacagagtgaaggcaccacaattgacgccataatctacgattctatgacagagtgggttttagacgttgctatcgaatttggtattgatggaggttcctttttcacacaagcatgtgttgtgaattctctatactaccatgtgcataaagggttaatctctttaccattgggtgaaactgtttcagttccaggttttccagtgttacaacgttgggaaaccccattgatcttacaaaatcatgaacaaatacaatcaccttggtcccagatgttgtttggtcaattcgctaacatcgatcaagcaagatgggtctttactaattcattctataagttagaggaagaggtaattgaatggactaggaagatctggaatttgaaagtcattggtccaacattgccatcaatgtatttggacaaaagacttgatgatgataaagataatggtttcaatttgtacaaggctaatcatcacgaatgtatgaattggctggatgacaaaccaaaggaatcagttgtatatgttgctttcggctctcttgttaaacatggtccagaacaagttgaggagattacaagagcacttatagactctgacgtaaactttttgtgggtcattaagcacaaagaggaggggaaactgccagaaaacctttctgaagtgataaagaccggaaaaggtctaatcgttgcttggtgtaaacaattggatgttttagctcatgaatctgtaggctgttttgtaacacattgcggattcaactctacactagaagccatttccttaggcgtacctgtcgttgcaatgcctcagttctccgatcagacaaccaacgctaaacttttggacgaaatactaggggtgggtgtcagagttaaagcagacgagaatggtatcgtcagaagagggaacctagcttcatgtatcaaaatgatcatggaagaggaaagaggagttatcataaggaaaaacgcagttaagtggaaggatcttgcaaaggttgccgtccatgaaggcggctcttcagataatgatattgttgaatttgtgtccgaactaatcaaagcctaa

SEQ ID NO: 4 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 5 - DNA, 1446 bp, Stevia rebaudiana
atggatgcaatggctacaactgagaagaaaccacacgtcatcttcataccatttccagcacaaagccacattaaagccatgctcaaactagcacaacttctccaccacaaaggactccagataaccttcgtcaacaccgacttcatccacaaccagtttcttgaatcatcgggcccacattgtctagacggtgcaccgggtttccggttcgaaaccattccggatggtgtttctcacagtccggaagcgagcatcccaatcagagaatcactcttgagatccattgaaaccaacttcttggatcgtttcattgatcttgtaaccaaacttccggatcctccgacttgtattatctcagatgggttcttgtcggttttcacaattgacgctgcaaaaaagcttggaattccggtcatgatgtattggacacttgctgcctgtgggttcatgggtttttaccatattcattctctcattgagaaaggatttgcaccacttaaagatgcaagttacttgacaaatgggtatttggacaccgtcattgattgggttccgggaatggaaggcatccgtctcaaggatttcccgctggactggagcactgacctcaatgacaaagttttgatgttcactacggaagctcctcaaaggtcacacaaggtttcacatcatattttccacacgttcgatgagttggagcctagtattataaaaactttgtcattgaggtataatcacatttacaccatcggcccactgcaattacttcttgatcaaatacccgaagagaaaaagcaaactggaattacgagtctccatggatacagtttagtaaaagaagaaccagagtgtttccagtggcttcagtctaaagaaccaaattccgtcgtttatgtaaattttggaagtactacagtaatgtctttagaagacatgacggaatttggttggggacttgctaatagcaaccattatttcctttggatcatccgatcaaacttggtgataggggaaaatgcagttttgccccctgaacttgaggaacatataaagaaaagaggctttattgctagctggtgttcacaagaaaaggtcttgaagcacccttcggttggagggttcttgactcattgtgggtggggatcgaccatcgagagcttgtctgctggggtgccaatgatatgctggccttattcgtgggaccagctgaccaactgtaggtatatatgcaaagaatgggaggttgggctcgagatgggaaccaaagtgaaacgagatgaagtcaagaggcttgtacaagagttgatgggagaaggaggtcacaaaatgaggaacaaggctaaagattggaaagaaaaggctcgcattgcaatagctcctaacggttcatcttctttgaacatagacaaaatggtcaaggaaatcaccgtgctagcaagaaactag

SEQ ID NO: 6 - DNA, 1446 bp, Artificial Sequence
  Codon-optimized UGT85C2
atggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaa

SEQ ID NO: 7 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 8 - DNA, 1377 bp, Artificial Sequence
  Codon-optimized UGT76G1
atggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 9 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 10 - DNA, 1422 bp, Artificial Sequence
  Codon-optimized UGT91D2e
atggctacatctgattctattgttgatgacaggaagcagttgcatgtggctactttcccttggcttgctttcggtcatatactgccttacctacaactatcaaaactgatagctgaaaaaggacataaagtgtcattcctttcaacaactagaaacattcaaagattatcttcccacatatcaccattgattaacgtcgttcaattgacacttccaagagtacaggaattaccagaagatgctgaagctacaacagatgtgcatcctgaagatatcccttacttgaaaaaggcatccgatggattacagcctgaggtcactagattccttgagcaacacagtccagattggatcatatacgactacactcactattggttgccttcaattgcagcatcactaggcatttctagggcacatttcagtgtaaccacaccttgggccattgcttacatgggtccatccgctgatgctatgattaacggcagtgatggtagaactaccgttgaagatttgacaaccccaccaaagtggtttccatttccaactaaagtctgttggagaaaacacgacttagcaagactggttccatacaaggcaccaggaatctcagacggctatagaatgggtttagtccttaaagggtctgactgcctattgtctaagtgttaccatgagtttgggacacaatggctaccacttttggaaacattacaccaagttcctgtcgtaccagttggtctattacctccagaaatccctggtgatgagaaggacgagacttgggtttcaatcaaaaagtggttagacgggaagcaaaaaggctcagtggtatatgtggcactgggttccgaagttttagtatctcaaacagaagttgtggaacttgccttaggtttggaactatctggattgccatttgtctgggcctacagaaaaccaaaaggccctgcaaagtccgattcagttgaattgccagacggctttgtcgagagaactagagatagagggttggtatggacttcatgggctccacaattgagaatcctgagtcacgaatctgtgtgcggtttcctaacacattgtggttctggttctatagttgaaggactgatgtttggtcatccacttatcatgttgccaatctttggtgaccagcctttgaatgcacgtctgttagaagataaacaagttggaattgaaatcccacgtaatgaggaagatggatgtttaaccaaggagtctgtggccagatcattacgttccgttgtcgttgaaaaggaaggcgaaatctacaaggccaatgcccgtgaactttcaaagatctacaatgacacaaaagtagagaaggaatatgtttctcaatttgtagattacctagagaaaaacgctagagccgtagctattgatcatgaatcctaa

SEQ ID NO: 11 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 12 - DNA, 1422 bp, Artificial Sequence
  Codon-optimized UGT91D2e-b
atggctacttctgattccatcgttgacgatagaaagcaattgcatgttgctacttttccatggttggctttcggtcatattttgccatacttgcaattgtccaagttgattgctgaaaagggtcacaaggtttcattcttgtctaccaccagaaacatccaaagattgtcctctcatatctccccattgatcaacgttgttcaattgactttgccaagagtccaagaattgccagaagatgctgaagctactactgatgttcatccagaagatatcccttacttgaaaaaggcttccgatggtttacaaccagaagttactagattcttggaacaacattccccagattggatcatctacgattatactcattactggttgccatccattgctgcttcattgggtatttctagagcccatttctctgttactactccatgggctattgcttatatgggtccatctgctgatgctatgattaacggttctgatggtagaactaccgttgaagatttgactactccaccaaagtggtttccatttccaacaaaagtctgttggagaaaacacgatttggctagattggttccatacaaagctccaggtatttctgatggttacagaatgggtatggttttgaaaggttccgattgcttgttgtctaagtgctatcatgaattcggtactcaatggttgcctttgttggaaacattgcatcaagttccagttgttccagtaggtttgttgccaccagaaattccaggtgacgaaaaagacgaaacttgggtttccatcaaaaagtggttggatggtaagcaaaagggttctgttgtttatgttgctttgggttccgaagctttggtttctcaaaccgaagttgttgaattggctttgggtttggaattgtctggtttgccatttgtttgggcttacagaaaacctaaaggtccagctaagtctgattctgttgaattgccagatggtttcgttgaaagaactagagatagaggtttggtttggacttcttgggctccacaattgagaattttgtctcatgaatccgtctgtggtttcttgactcattgtggttctggttctatcgttgaaggtttgatgtttggtcacccattgattatgttgccaatctttggtgaccaaccattgaacgctagattattggaagataagcaagtcggtatcgaaatcccaagaaatgaagaagatggttgcttgaccaaagaatctgttgctagatctttgagatccgttgtcgttgaaaaagaaggtgaaatctacaaggctaacgctagagaattgtccaagatctacaacgataccaaggtcgaaaaagaatacgtttcccaattcgttgactacttggaaaagaatgctagagctgttgccattgatcatgaatcttga

SEQ ID NO: 13 - protein, 473 aa, Artificial Sequence
  UGT91D2e-b
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 14 - DNA, 1389 bp, Oryza sativa
atggactccggctactcctcctcctacgccgccgccgccgggatgcacgtcgtgatctgcccgtggctcgccttcggccacctgctcccgtgcctcgacctcgcccagcgcctcgcgtcgcggggccaccgcgtgtcgttcgtctccacgccgcggaacatatcccgcctcccgccggtgcgccccgcgctcgcgccgctcgtcgccttcgtggcgctgccgctcccgcgcgtcgaggggctccccgacggcgccgagtccaccaacgacgtcccccacgacaggccggacatggtcgagctccaccggagggccttcgacgggctcgccgcgcccttctcggagttcttgggcaccgcgtgcgccgactgggtcatcgtcgacgtcttccaccactgggccgcagccgccgctctcgagcacaaggtgccatgtgcaatgatgttgttgggctctgcacatatgatcgcttccatagcagacagacggctcgagcgcgcggagacagagtcgcctgcggctgccgggcagggacgcccagcggcggcgccaacgttcgaggtggcgaggatgaagttgatacgaaccaaaggctcatcgggaatgtccctcgccgagcgcttctccttgacgctctcgaggagcagcctcgtcgtcgggcggagctgcgtggagttcgagccggagaccgtcccgctcctgtcgacgctccgcggtaagcctattaccttccttggccttatgccgccgttgcatgaaggccgccgcgaggacggcgaggatgccaccgtccgctggctcgacgcgcagccggccaagtccgtcgtgtacgtcgcgctaggcagcgaggtgccactgggagtggagaaggtccacgagctcgcgctcgggctggagctcgccgggacgcgcttcctctgggctcttaggaagcccactggcgtctccgacgccgacctcctccccgccggcttcgaggagcgcacgcgcggccgcggcgtcgtggcgacgagatgggttcctcagatgagcatactggcgcacgccgccgtgggcgcgttcctgacccactgcggctggaactcgaccatcgaggggctcatgttcggccacccgcttatcatgctgccgatcttcggcgaccagggaccgaacgcgcggctaatcgaggcgaagaacgccggattgcaggtggcaagaaacgacggcgatggatcgttcgaccgagaaggcgtcgcggcggcgattcgtgcagtcgcggtggaggaagaaagcagcaaagtgtttcaagccaaagccaagaagctgcaggagatcgtcgcggacatggcctgccatgagaggtacatcgacggattcattcagcaattgagatcttacaaggattga

SEQ ID NO: 15 - DNA, 1389 bp, Artificial Sequence
  Codon-optimized EUGT11
atggatagtggctactcctcatcttatgctgctgccgctggtatgcacgttgtgatctgcccttggttggcctttggtcacctgttaccatgtctggatttagcccaaagactggcctcaagaggccatagagtatcatttgtgtctactcctagaaatatctctcgtttaccaccagtcagacctgctctagctcctctagttgcattcgttgctcttccacttccaagagtagaaggattgccagacggcgctgaatctactaatgacgtaccacatgatagacctgacatggtcgaattgcatagaagagcctttgatggattggcagctccattttctgagttcctgggcacagcatgtgcagactgggttatagtcgatgtatttcatcactgggctgctgcagccgcattggaacataaggtgccttgtgctatgatgttgttagggtcagcacacatgatcgcatccatagctgatagaagattggaaagagctgaaacagaatccccagccgcagcaggacaaggtaggccagctgccgccccaacctttgaagtggctagaatgaaattgattcgtactaaaggtagttcagggatgagtcttgctgaaaggttttctctgacattatctagatcatcattagttgtaggtagatcctgcgtcgagttcgaacctgaaacagtacctttactatctactttgagaggcaaacctattactttccttggtctaatgcctccattacatgaaggaaggagagaagatggtgaagatgctactgttaggtggttagatgcccaacctgctaagtctgttgtttacgttgcattgggttctgaggtaccactaggggtggaaaaggtgcatgaattagcattaggacttgagctggccggaacaagattcctttgggctttgagaaaaccaaccggtgtttctgacgccgacttgctaccagctgggttcgaagagagaacaagaggccgtggtgtcgttgctactagatgggtcccacaaatgagtattctagctcatgcagctgtaggggcctttctaacccattgcggttggaactcaacaatagaaggactgatgtttggtcatccacttattatgttaccaatctttggcgatcagggacctaacgcaagattgattgaggcaaagaacgcaggtctgcaggttgcacgtaatgatggtgatggttcctttgatagagaaggcgttgcagctgccatcagagcagtcgccgttgaggaagagtcatctaaagttttccaagctaaggccaaaaaattacaagagattgtggctgacatggcttgtcacgaaagatacatcgatggtttcatccaacaattgagaagttataaagactaa

SEQ ID NO: 16 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 17 - protein, 465 aa, Artificial Sequence
  UGT91D2e-b-EUGT11 chimera 3
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIARNDGDGSFDREGVAAAIRAVAVEEESSKVFQAKAKKLQEIVADMACHERYIDGFIQQLRSYKD

SEQ ID NO: 18 - protein, 470 aa, Artificial Sequence
  UGT91D2e-b-EUGT11 chimera 7
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVPRNEEDGCLTKESVARSLRSVVVEKEGEIYKANARELSKIYNDTKVEKEYVSQFVDYLEKNARAVAIDHES

SEQ ID NO: 19 - DNA, 1086 bp, Artificial Sequence
  Codon-optimized GGPPS
atggctttggtaaacccaaccgctcttttctatggtacctctatcagaacaagacctacaaacttactaaatccaactcaaaagctaagaccagtttcatcatcttccttaccttctttctcatcagttagtgcgattcttactgaaaaacatcaatctaatccttctgagaacaacaatttgcaaactcatctagaaactcctttcaactttgatagttatatgttggaaaaagtcaacatggttaacgaggcgcttgatgcatctgtcccactaaaagacccaatcaaaatccatgaatccatgagatactctttattggcaggcggtaagagaatcagaccaatgatgtgtattgcagcctgcgaaatagtcggaggtaatatccttaacgccatgccagccgcatgtgccgtggaaatgattcatactatgtctttggtgcatgacgatcttccatgtatggataatgatgacttcagaagaggtaaacctatttcacacaaggtctacggggaggaaatggcagtattgaccggcgatgctttactaagtttatctttcgaacatatagctactgctacaaagggtgtatcaaaggatagaatcgtcagagctataggggagttggcccgttcagttggctccgaaggtttagtggctggacaagttgtagatatcttgtcagagggtgctgatgttggattagatcacctagaatacattcacatccacaaaacagcaatgttgcttgagtcctcagtagttattggcgctatcatgggaggaggatctgatcagcagatcgaaaagttgagaaaattcgctagatctattggtctactattccaagttgtggatgacattttggatgttacaaaatctaccgaagagttggggaaaacagctggtaaggatttgttgacagataagacaacttacccaaagttgttaggtatagaaaagtccagagaatttgccgaaaaacttaacaaggaagcacaagagcaattaagtggctttgatagacgtaaggcagctcctttgatcgcgttagccaactacaatgcgtaccgtcaaaattga

SEQ ID NO: 20 - protein, 361 aa, Stevia rebaudiana
MALVNPTALFYGTSIRTRPTNLLNPTQKLRPVSSSSLPSFSSVSAILTEKHQSNPSENNNLQTHLETPFNFDSYMLEKVNMVNEALDASVPLKDPIKIHESMRYSLLAGGKRIRPMMCIAACEIVGGNILNAMPAACAVEMIHTMSLVHDDLPCMDNDDFRRGKPISHKVYGEEMAVLTGDALLSLSFEHIATATKGVSKDRIVRAIGELARSVGSEGLVAGQVVDILSEGADVGLDHLEYIHIHKTAMLLESSVVIGAIMGGGSDQQIEKLRKFARSIGLLFQVVDDILDVTKSTEELGKTAGKDLLTDKTTYPKLLGIEKSREFAEKLNKEAQEQLSGFDRRKAAPLIALANYNAYRQN

SEQ ID NO: 21 - DNA, 1029 bp, Artificial Sequence
  Codon-optimized GGPPS
atggctgagcaacaaatatctaacttgctgtctatgtttgatgcttcacatgctagtcagaaattagaaattactgtccaaatgatggacacataccattacagagaaacgcctccagattcctcatcttctgaaggcggttcattgtctagatacgacgagagaagagtctctttgcctctcagtcataatgctgcctctccagatattgtatcacaactatgtttttccactgcaatgtcttcagagttgaatcacagatggaaatctcaaagattaaaggtggccgattctccttacaactatatcctaacattaccatcaaaaggaattagaggtgcctttatcgattccctgaacgtatggttggaggttccagaggatgaaacatcagtcatcaaggaagttattggtatgctccacaactcttcattaatcattgatgacttccaagataattctccacttagaagaggaaagccatctacccatacagtcttcggccctgcccaggctatcaatactgctacttacgttatagttaaagcaatcgaaaagatacaagacatagtgggacacgatgcattggcagatgttacgggtactattacaactattttccaaggtcaggccatggacttgtggtggacagcaaatgcaatcgttccatcaatacaggaatacttacttatggtaaacgataaaaccggtgctctctttagactgagtttggagttgttagctctgaattccgaagccagtatttctgactctgctttagaaagtttatctagtgctgtttccttgctaggtcaatacttccaaatcagagacgactatatgaacttgatcgataacaagtatacagatcagaaaggcttctgcgaagatcttgatgaaggcaagtactcactaacacttattcatgccctccaaactgattcatccgatctactgaccaacatcctttcaatgagaagagtgcaaggaaagttaacggcacaaaagagatgttggttctggaaatga

SEQ ID NO: 22 - protein, 342 aa, Gibberella fujikuroi
MAEQQISNLLSMFDASHASQKLEITVQMMDTYHYRETPPDSSSSEGGSLSRYDERRVSLPLSHNAASPDIVSQLCFSTAMSSELNHRWKSQRLKVADSPYNYILTLPSKGIRGAFIDSLNVWLEVPEDETSVIKEVIGMLHNSSLIIDDFQDNSPLRRGKPSTHTVFGPAQAINTATYVIVKAIEKIQDIVGHDALADVTGTITTIFQGQAMDLWWTANAIVPSIQEYLLMVNDKTGALFRLSLELLALNSEASISDSALESLSSAVSLLGQYFQIRDDYMNLIDNKYTDQKGFCEDLDEGKYSLTLIHALQTDSSDLLTNILSMRRVQGKLTAQKRCWFWK

SEQ ID NO: 23 - DNA, 903 bp, Artificial Sequence
  Codon-optimized GGPPS
atggaaaagactaaggagaaagcagaacgtatcttgctggagccatacagatacttattacaactaccaggaaagcaagtccgttctaaactatcacaagcgttcaatcactggttaaaagttcctgaagataagttacaaatcattattgaagtcacagaaatgctacacaatgcttctttactgatcgatgatatagaggattcttccaaactgagaagaggttttcctgtcgctcattccatatacggggtaccaagtgtaatcaactcagctaattacgtctacttcttgggattggaaaaagtattgacattagatcatccagacgctgtaaagctattcaccagacaacttcttgaattgcatcaaggtcaaggtttggatatctattggagagacacttatacttgcccaacagaagaggagtacaaagcaatggttctacaaaagactggcggtttgttcggacttgccgttggtctgatgcaacttttctctgattacaaggaggacttaaagcctctgttggataccttgggcttgtttttccagattagagatgactacgctaacttacattcaaaggaatattcagaaaacaaatcattctgtgaagatttgactgaagggaagtttagttttccaacaatccacgccatttggtcaagaccagaatctactcaagtgcaaaacattctgcgtcagagaacagagaatattgacatcaaaaagtattgtgttcagtacttggaagatgttggttcttttgcttacacaagacatacacttagagaattagaggcaaaagcatacaagcaaatagaagcctgtggaggcaatccttctctagtggcattggttaaacatttgtccaaaatgttcaccgaggaaaacaagtaa

SEQ ID NO: 24 - protein, 300 aa, Mus musculus
MEKTKEKAERILLEPYRYLLQLPGKQVRSKLSQAFNHWLKVPEDKLQIIIEVTEMLHNASLLIDDIEDSSKLRRGFPVAHSIYGVPSVINSANYVYFLGLEKVLTLDHPDAVKLFTRQLLELHQGQGLDIYWRDTYTCPTEEEYKAMVLQKTGGLFGLAVGLMQLFSDYKEDLKPLLDTLGLFFQIRDDYANLHSKEYSENKSFCEDLTEGKFSFPTIHAIWSRPESTQVQNILRQRTENIDIKKYCVQYLEDVGSFAYTRHTLRELEAKAYKQIEACGGNPSLVALVKHLSKMFTEENK

SEQ ID NO: 25 - DNA, 1020 bp, Artificial Sequence
  Codon-optimized GGPPS
atggcaagattctattttcttaacgcactattgatggttatctcattacaatcaactacagccttcactccagctaaacttgcttatccaacaacaacaacagctctaaatgtcgcctccgccgaaacttctttcagtctagatgaatacttggcctctaagataggacctatagagtctgccttggaagcatcagtcaaatccagaattccacagaccgataagatctgcgaatctatggcctactctttgatggcaggaggcaagagaattagaccagtgttgtgtatcgctgcatgtgagatgttcggtggatcccaagatgtcgctatgcctactgctgtggcattagaaatgatacacacaatgtctttgattcatgatgatttgccatccatggataacgatgacttgagaagaggtaaaccaacaaaccatgtcgttttcggcgaagatgtagctattcttgcaggtgactctttattgtcaacttccttcgagcacgtcgctagagaaacaaaaggagtgtcagcagaaaagatcgtggatgttatcgctagattaggcaaatctgttggtgccgagggccttgctggcggtcaagttatggacttagaatgtgaagctaaaccaggtaccacattagacgacttgaaatggattcatatccataaaaccgctacattgttacaagttgctgtagcttctggtgcagttctaggtggtgcaactcctgaagaggttgctgcatgcgagttgtttgctatgaatataggtcttgcctttcaagttgccgacgatatccttgatgtaaccgcttcatcagaagatttgggtaaaactgcaggcaaagatgaagctactgataagacaacttacccaaagttattaggattagaagagagtaaggcatacgcaagacaactaatcgatgaagccaaggaaagtttggctccttttggagatagagctgcccctttattggccattgcagatttcattattgatagaaagaattga

SEQ ID NO: 26 - protein, 339 aa, Thalassiosira pseudonana
MARFYFLNALLMVISLQSTTAFTPAKLAYPTTTTALNVASAETSFSLDEYLASKIGPIESALEASVKSRIPQTDKICESMAYSLMAGGKRIRPVLCIAACEMFGGSQDVAMPTAVALEMIHTMSLIHDDLPSMDNDDLRRGKPTNHVVFGEDVAILAGDSLLSTSFEHVARETKGVSAEKIVDVIARLGKSVGAEGLAGGQVMDLECEAKPGTTLDDLKWIHIHKTATLLQVAVASGAVLGGATPEEVAACELFAMNIGLAFQVADDILDVTASSEDLGKTAGKDEATDKTTYPKLLGLEESKAYARQLIDEAKESLAPFGDRAAPLLAIADFIIDRKN

SEQ ID NO: 27 - DNA, 1068 bp, Artificial Sequence
  Codon-optimized GGPPS
atgcacttagcaccacgtagagtccctagaggtagaagatcaccacctgacagagttcctgaaagacaaggtgccttgggtagaagacgtggagctggctctactggctgtgcccgtgctgctgctggtgttcaccgtagaagaggaggaggcgaggctgatccatcagctgctgtgcatagaggctggcaagccggtggtggcaccggtttgcctgatgaggtggtgtctaccgcagccgccttagaaatgtttcatgcttttgctttaatccatgatgatatcatggatgatagtgcaactagaagaggctccccaactgttcacagagccctagctgatcgtttaggcgctgctctggacccagatcaggccggtcaactaggagtttctactgctatcttggttggagatctggctttgacatggtccgatgaattgttatacgctccattgactccacatagactggcagcagtactaccattggtaacagctatgagagctgaaaccgttcatggccaatatcttgatataactagtgctagaagacctgggaccgatacttctcttgcattgagaatagccagatataagacagcagcttacacaatggaacgtccactgcacattggtgcagccctggctggggcaagaccagaactattagcagggctttcagcatacgccttgccagctggagaagccttccaattggcagatgacctgctaggcgtcttcggtgatccaagacgtacagggaaacctgacctagatgatcttagaggtggaaagcatactgtcttagtcgccttggcaagagaacatgccactccagaacagagacacacattggatacattattgggtacaccaggtcttgatagacaaggcgcttcaagactaagatgcgtattggtagcaactggtgcaagagccgaagccgaaagacttattacagagagaagagatcaagcattaactgcattgaacgcattaacactgccacctcctttagctgaggcattagcaagattgacattagggtctacagctcatcctgcctaa

SEQ ID NO: 28 - protein, 355 aa, Streptomyces clavuligerus
MHLAPRRVPRGRRSPPDRVPERQGALGRRRGAGSTGCARAAAGVHRRRGGGEADPSAAVHRGWQAGGGTGLPDEVVSTAAALEMFHAFALIHDDIMDDSATRRGSPTVHRALADRLGAALDPDQAGQLGVSTAILVGDLALTWSDELLYAPLTPHRLAAVLPLVTAMRAETVHGQYLDITSARRPGTDTSLALRIARYKTAAYTMERPLHIGAALAGARPELLAGLSAYALPAGEAFQLADDLLGVFGDPRRTGKPDLDDLRGGKHTVLVALAREHATPEQRHTLDTLLGTPGLDRQGASRLRCVLVATGARAEAERLITERRDQALTALNALTLPPPLAEALARLTLGSTAHPA

SEQ ID NO: 29 - DNA, 993 bp, Artificial Sequence
  Codon-optimized GGPPS
atgtcatatttcgataactacttcaatgagatagttaattccgtgaacgacatcattaagtcttacatctctggcgacgtaccaaaactatacgaagcctcctaccatttgtttacatcaggaggaaagagactaagaccattgatccttacaatttcttctgatcttttcggtggacagagagaaagagcatactatgctggcgcagcaatcgaagttttgcacacattcactttggttcacgatgatatcatggatcaagataacattcgtagaggtcttcctactgtacatgtcaagtatggcctacctttggccattttagctggtgacttattgcatgcaaaagcctttcaattgttgactcaggcattgagaggtctaccatctgaaactatcatcaaggcgtttgatatctttacaagatctatcattatcatatcagaaggtcaagctgtcgatatggaattcgaagatagaattgatatcaaggaacaagagtatttggatatgatatctcgtaaaaccgctgccttattctcagcttcttcttccattggggcgttgatagctggagctaatgataacgatgtgagattaatgtccgatttcggtacaaatcttgggatcgcatttcaaattgtagatgatatacttggtttaacagctgatgaaaaagagctaggaaaacctgttttcagtgatatcagagaaggtaaaaagaccatattagtcattaagactttagaattgtgtaaggaagacgagaaaaagattgtgttaaaagcgctaggcaacaagtcagcatcaaaggaagagttgatgagttctgctgacataatcaaaaagtactcattggattacgcctacaacttagctgagaaatactacaaaaacgccatcgattctctaaatcaagtttcaagtaaaagtgatattccagggaaggcattgaaatatcttgctgaattcaccatcagaagacgtaagtaa

SEQ ID NO: 30 - protein, 330 aa, Sulfolobus acidocaldarius
MSYFDNYFNEIVNSVNDIIKSYISGDVPKLYEASYHLFTSGGKRLRPLILTISSDLFGGQRERAYYAGAAIEVLHTFTLVHDDIMDQDNIRRGLPTVHVKYGLPLAILAGDLLHAKAFQLLTQALRGLPSETIIKAFDIFTRSIIIISEGQAVDMEFEDRIDIKEQEYLDMISRKTAALFSASSSIGALIAGANDNDVRLMSDFGTNLGIAFQIVDDILGLTADEKELGKPVFSDIREGKKTILVIKTLELCKEDEKKIVLKALGNKSASKEELMSSADIIKKYSLDYAYNLAEKYYKNAIDSLNQVSSKSDIPGKALKYLAEFTIRRRK

SEQ ID NO: 31 - DNA, 894 bp, Artificial Sequence
  Codon-optimized GGPPS
atggtcgcacaaactttcaacctggatacctacttatcccaaagacaacaacaagttgaagaggccctaagtgctgctcttgtgccagcttatcctgagagaatatacgaagctatgagatactccctcctggcaggtggcaaaagattaagacctatcttatgtttagctgcttgcgaattggcaggtggttctgttgaacaagccatgccaactgcgtgtgcacttgaaatgatccatacaatgtcactaattcatgatgacctgccagccatggataacgatgatttcagaagaggaaagccaactaatcacaaggtgttcggggaagatatagccatcttagcgggtgatgcgcttttagcttacgcttttgaacatattgcttctcaaacaagaggagtaccacctcaattggtgctacaagttattgctagaatcggacacgccgttgctgcaacaggcctcgttggaggccaagtcgtagaccttgaatctgaaggtaaagctatttccttagaaacattggagtatattcactcacataagactggagccttgctggaagcatcagttgtctcaggcggtattctcgcaggggcagatgaagagcttttggccagattgtctcattacgctagagatataggcttggcttttcaaatcgtcgatgatatcctggatgttactgctacatctgaacagttggggaaaaccgctggtaaagaccaggcagccgcaaaggcaacttatccaagtctattgggtttagaagcctctagacagaaagcggaagagttgattcaatctgctaaggaagccttaagaccttacggttcacaagcagagccactcctagcgctggcagacttcatcacacgtcgtcagcattaa

SEQ ID NO: 32 - protein, 297 aa, Synechococcus sp.
MVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQH

SEQ ID NO: 33 - DNA, 1680 bp, Artificial Sequence
  Codon-optimized CDPS
atgaaaaccgggtttatctcaccagcaacagtatttcatcacagaatctcaccagcgaccactttcagacatcacttatcacctgctactacaaactctacaggcattgtcgccttaagagacatcaacttcagatgtaaagcagtttctaaagagtactctgatctgttgcagaaagatgaggcttctttcacaaaatgggacgatgacaaggtgaaagatcatcttgataccaacaaaaacttatacccaaatgatgagattaaggaatttgttgaatcagtaaaggctatgttcggtagtatgaatgacggggagataaacgtctctgcatacgatactgcatgggttgctttggttcaagatgtcgatggatcaggtagtcctcagttcccttcttctttagaatggattgccaacaatcaattgtcagatggatcatggggagatcatttgctgttctcagctcacgatagaatcatcaacacattagcatgcgttattgcacttacaagttggaatgttcatccttctaagtgtgaaaaaggtttgaattttctgagagaaaacatttgcaaattagaagatgaaaacgcagaacatatgccaattggttttgaagtaacattcccatcactaattgatatcgcgaaaaagttgaacattgaagtacctgaggatactccagcacttaaagagatctacgcacgtagagatatcaagttaactaagatcccaatggaagttcttcacaaggtacctactactttgttacattctttggaaggaatgcctgatttggagtgggaaaaactgttaaagctacaatgtaaagatggtagtttcttgttttccccatctagtaccgcattcgccctaatgcaaacaaaagatgagaaatgcttacagtatctaacaaatatcgtcactaagttcaacggtggcgtgcctaatgtgtacccagtcgatttgtttgaacatatttgggttgttgatagactgcagagattggggattgccagatacttcaaatcagagataaaagattgtgtagagtatatcaataagtactggaccaaaaatggaatttgttgggctagaaatactcacgttcaagatatcgatgatacagccatgggattcagagtgttgagagcgcacggttatgacgtcactccagatgtttttagacaatttgaaaaagatggtaaattcgtttgctttgcagggcaatcaacacaagccgtgacaggaatgtttaacgtttacagagcctctcaaatgttgttcccaggggagagaattttggaagatgccaaaaagttctcttacaattacttaaaggaaaagcaaagtaccaacgaattgctggataaatggataatcgctaaagatctacctggtgaagttggttatgctctggatatcccatggtatgcttccttaccaagattggaaactcgttattaccttgaacaatacggcggtgaagatgatgtctggataggcaagacattatacagaatgggttacgtgtccaataacacatatctagaaatggcaaagctggattacaataactatgttgcagtccttcaattagaatggtacacaatacaacaatggtacgtcgatattggtatagagaagttcgaatctgacaacatcaagtcagtcctg

SEQ ID NO: 34 - protein, 787 aa, Stevia rebaudiana
MKTGFISPATVFHHRISPATTFRHHLSPATTNSTGIVALRDINFRCKAVSKEYSDLLQKDEASFTKWDDDKVKDHLDTNKNLYPNDEIKEFVESVKAMFGSMNDGEINVSAYDTAWVALVQDVDGSGSPQFPSSLEWIANNQLSDGSWGDHLLFSAHDRIINTLACVIALTSWNVHPSKCEKGLNFLRENICKLEDENAEHMPIGFEVTFPSLIDIAKKLNIEVPEDTPALKEIYARRDIKLTKIPMEVLHKVPTTLLHSLEGMPDLEWEKLLKLQCKDGSFLFSPSSTAFALMQTKDEKCLQYLTNIVTKFNGGVPNVYPVDLFEHIWVVDRLQRLGIARYFKSEIKDCVEYINKYWTKNGICWARNTHVQDIDDTAMGFRVLRAHGYDVTPDVFRQFEKDGKFVCFAGQSTQAVTGMFNVYRASQMLFPGERILEDAKKFSYNYLKEKQSTNELLDKWIIAKDLPGEVGYALDIPWYASLPRLETRYYLEQYGGEDDVWIGKTLYRMGYVSNNTYLEMAKLDYNNYVAVLQLEWYTIQQWYVDIGIEKFESDNIKSVLVSYYLAAASIFEPERSKERIAWAKTTILVDKITSIFDSSQSSKEDITAFIDKFRNKSSSKKHSINGEPWHEVMVALKKTLHGFALDALMTHSQDIHPQLHQAWEMWLTKLQDGVDVTAELMVQMINMTAGRWVSKELLTHPQYQRLSTVTNSVCHDITKLHNFKENSTTVDSKVQELVQLVFSDTPDDLDQDMKQTFLTVMKTFYYKAWCDPNTINDHISKVFEIVI

SEQ ID NO: 35 - DNA, 1584 bp, Artificial Sequence
  Codon-optimized CDPS
atgcctgatgcacacgatgctccacctccacaaataagacagagaacactagtagatgaggctacccaactgctaactgagtccgcagaagatgcatggggtgaagtcagtgtgtcagaatacgaaacagcaaggctagttgcccatgctacatggttaggtggacacgccacaagagtggccttccttctggagagacaacacgaagacgggtcatggggtccaccaggtggatataggttagtccctacattatctgctgttcacgcattattgacatgtcttgcctctcctgctcaggatcatggcgttccacatgatagacttttaagagctgttgacgcaggcttgactgccttgagaagattggggacatctgactccccacctgatactatagcagttgagctggttatcccatctttgctagagggcattcaacacttactggaccctgctcatcctcatagtagaccagccttctctcaacatagaggctctcttgtttgtcctggtggactagatgggagaactctaggagctttgagatcacacgccgcagcaggtacaccagtaccaggaaaagtctggcacgcttccgagactttgggcttgagtaccgaagctgcttctcacttgcaaccagcccaaggtataatcggtggctctgctgctgccacagcaacatggctaaccagggttgcaccatctcaacagtcagattctgccagaagataccttgaggaattacaacacagatactctggcccagttccttccattacccctatcacatacttcgaaagagcatggttattgaacaattttgcagcagccggtgttccttgtgaggctccagctgctttgttggattccttagaagcagcacttacaccacaaggtgctcctgctggagcaggattgcctccagatgctgatgatacagccgctgtgttgcttgcattggcaacacatgggagaggtagaagaccagaagtactgatggattacaggactgacgggtatttccaatgctttattggggaaaggactccatcaatttcaacaaacgctcacgtattggaaacattagggcatcatgtggcccaacatccacaagatagagccagatacggatcagccatggataccgcatcagcttggctgctggcagctcaaaagcaagatggctcttggttagataaatggcatgcctcaccatactacgctactgtttgttgcacacaagccctagccgctcatgcaagtcctgcaactgcaccagctagacagagagctgtcagatgggttttagccacacaaagatccgatggcggttggggtctatggcattcaactgttgaagagactgcttatgccttacagatcttggccccaccttctggtggtggcaatatcccagtccaacaagcacttactagaggcagagcaagattgtgtggagccttgccactgactcctttatggcatgataaggatttgtatactccagtaagagtagtcagagctgccagagctgctgctctgtacactaccagagatctattgttaccaccattgtaa

SEQ ID NO: 36 - protein, 527 aa, Streptomyces clavuligerus
MPDAHDAPPPQIRQRTLVDEATQLLTESAEDAWGEVSVSEYETARLVAHATWLGGHATRVAFLLERQHEDGSWGPPGGYRLVPTLSAVHALLTCLASPAQDHGVPHDRLLRAVDAGLTALRRLGTSDSPPDTIAVELVIPSLLEGIQHLLDPAHPHSRPAFSQHRGSLVCPGGLDGRTLGALRSHAAAGTPVPGKVWHASETLGLSTEAASHLQPAQGIIGGSAAATATWLTRVAPSQQSDSARRYLEELQHRYSGPVPSITPITYFERAWLLNNFAAAGVPCEAPAALLDSLEAALTPQGAPAGAGLPPDADDTAAVLLALATHGRGRRPEVLMDYRTDGYFQCFIGERTPSISTNAHVLETLGHHVAQHPQDRARYGSAMDTASAWLLAAQKQDGSWLDKWHASPYYATVCCTQALAAHASPATAPARQRAVRWVLATQRSDGGWGLWHSTVEETAYALQILAPPSGGGNIPVQQALTRGRARLCGALPLTPLWHDKDLYTPVRVVRAARAAALYTTRDLLLPPL

SEQ ID NO: 37 - DNA, 1551 bp, Artificial Sequence
  Codon-optimized CDPS
atgaacgccctatccgaacacattttgtctgaattgagaagattattgtctgaaatgagtgatggcggatctgttggtccatctgtgtatgatacggcccaggccctaagattccacggtaacgtaacaggtagacaagatgcatatgcttggttgatcgcccagcaacaagcagatggaggttggggctctgccgactttccactctttagacatgctccaacatgggctgcacttctcgcattacaaagagctgatccacttcctggcgcagcagacgcagttcagaccgcaacaagattcttgcaaagacaaccagatccatacgctcatgccgttcctgaggatgcccctattggtgctgaactgatcttgcctcagttttgtggagaggctgcttggttgttgggaggtgtggccttccctagacacccagccctattaccattaagacaggcttgtttagtcaaactgggtgcagtcgccatgttgccttcaggacacccattgctccactcctgggaggcatggggtacttctccaacaacagcctgtccagacgatgatggttctataggtatctcaccagcagctacagccgcctggagagcccaggctgtgaccagaggctcaactcctcaagtgggcagagctgacgcatacttacaaatggcttcaagagcaacgagatcaggcatagaaggagtcttccctaatgtttggcctataaacgtattcgaaccatgctggtcactgtacactctccatcttgccggtctgttcgcccatccagcactggctgaggctgtaagagttatcgttgctcaacttgaagcaagattgggagtgcatggcctcggaccagctttacattttgctgccgacgctgatgatactgcagttgccttatgcgttctgcatttggctggcagagatcctgcagttgacgcattgagacattttgaaattggtgagctctttgttacattcccaggagagagaaatgctagtgtctctacgaacattcacgctcttcatgctttgagattgttaggtaaaccagctgccggagcaagtgcatacgtcgaagcaaatagaaatccacatggtttgtgggacaacgaaaaatggcacgtttcatggctttatccaactgcacacgccgttgcagctctagctcaaggcaagcctcaatggagagatgaaagagcactagccgctctactacaagctcaaagagatgatggtggttggggagctggtagaggatccactttcgaggaaaccgcctacgctcttttcgctttacacgttatggacggatctgaggaagccacaggcagaagaagaatcgctcaagtcgtcgcaagagccttagaatggatgctagctagacatgccgcacatggattaccacaaacaccactctggattggtaaggaattgtactgtcctactagagtcgtaagagtagctgagctagctggcctgtggttagcattaagatggggtagaagagtattagctgaaggtgctggtgctgcaccttaa

SEQ ID NO: 38 - protein, 516 aa, Bradyrhizobium japonicum
MNALSEHILSELRRLLSEMSDGGSVGPSVYDTAQALRFHGNVTGRQDAYAWLIAQQQADGGWGSADFPLFRHAPTWAALLALQRADPLPGAADAVQTATRFLQRQPDPYAHAVPEDAPIGAELILPQFCGEAAWLLGGVAFPRHPALLPLRQACLVKLGAVAMLPSGHPLLHSWEAWGTSPTTACPDDDGSIGISPAATAAWRAQAVTRGSTPQVGRADAYLQMASRATRSGIEGVFPNVWPINVFEPCWSLYTLHLAGLFAHPALAEAVRVIVAQLEARLGVHGLGPALHFAADADDTAVALCVLHLAGRDPAVDALRHFEIGELFVTFPGERNASVSTNIHALHALRLLGKPAAGASAYVEANRNPHGLWDNEKWHVSWLYPTAHAVAALAQGKPQWRDERALAALLQAQRDDGGWGAGRGSTFEETAYALFALHVMDGSEEATGRRRIAQVVARALEWMLARHAAHGLPQTPLWIGKELYCPTRVVRVAELAGLWLALRWGRRVLAEGAGAAP

SEQ ID NO: 39 - DNA, 2490 bp, Artificial Sequence
  Codon-optimized CDPS
atggttttgtcttcttcttgtactacagtaccacacttatcttcattagctgtcgtgcaacttggtccttggagcagtaggattaaaaagaaaaccgatactgttgcagtaccagccgctgcaggaaggtggagaagggccttggctagagcacagcacacatcagaatccgcagctgtcgcaaagggcagcagtttgacccctatagtgagaactgacgctgagtcaaggagaacaagatggccaaccgatgacgatgacgccgaacctttagtggatgagatcagggcaatgcttacttccatgtctgatggtgacatttccgtgagcgcatacgatacagcctgggtcggattggttccaagattagacggcggtgaaggtcctcaatttccagcagctgtgagatggataagaaataaccagttgcctgacggaagttggggcgatgccgcattattctctgcctatgacaggcttatcaatacccttgcctgcgttgtaactttgacaaggtggtccctagaaccagagatgagaggtagaggactatcttttttgggtaggaacatgtggaaattagcaactgaagatgaagagtcaatgcctattggcttcgaattagcatttccatctttgatagagcttgctaagagcctaggtgtccatgacttcccttatgatcaccaggccctacaaggaatctactcttcaagagagatcaaaatgaagaggattccaaaagaagtgatgcataccgttccaacatcaatattgcacagtttggagggtatgcctggcctagattgggctaaactacttaaactacagagcagcgacggaagttttttgttctcaccagctgccactgcatatgctttaatgaataccggagatgacaggtgttttagctacatcgatagaacagtaaagaaattcaacggcggcgtccctaatgtttatccagtggatctatttgaacatatttgggccgttgatagacttgaaagattaggaatctccaggtacttccaaaaggagatcgaacaatgcatggattatgtaaacaggcattggactgaggacggtatttgttgggcaaggaactctgatgtcaaagaggtggacgacacagctatggcctttagacttcttaggttgcacggctacagcgtcagtcctgatgtgtttaaaaacttcgaaaaggacggtgaatttttcgcatttgtcggacagtctaatcaagctgttaccggtatgtacaacttaaacagagcaagccagatatccttcccaggcgaggatgtgcttcatagagctggtgccttctcatatgagttcttgaggagaaaagaagcagagggagctttgagggacaagtggatcatttctaaagatctacctggtgaagttgtgtatactttggattttccatggtacggcaacttacctagagtcgaggccagagactacctagagcaatacggaggtggtgatgacgtttggattggcaagacattgtataggatgccacttgtaaacaatgatgtatatttggaattggcaagaatggatttcaaccactgccaggctttgcatcagttagagtggcaaggactaaaaagatggtatactgaaaataggttgatggactttggtgtcgcccaagaagatgcccttagagcttattttcttgcagccgcatctgtttacgagccttgtagagctgccgagaggcttgcatgggctagagccgcaatactagctaacgccgtgagcacccacttaagaaatagcccatcattcagagaaaggttagagcattctcttaggtgtagacctagtgaagagacagatggctcctggtttaactcctcaagtggctctgatgcagttttagtaaaggctgtcttaagacttactgattcattagccagggaagcacagccaatccatggaggtgacccagaagatattatacacaagttgttaagatctgcttgggccgagtgggttagggaaaaggcagacgctgccgatagcgtgtgcaatggtagttctgcagtagaacaagagggatcaagaatggtccatgataaacagacctgtctattattggctagaatgatcgaaatttctgccggtagggcagctggtgaagcagccagtgaggacggcgatagaagaataattcaattaacaggctccatctgcgacagtcttaagcaaaaaatgctagtttcacaggaccctgaaaaaaatgaagagatgatgtctcacgtggatgacgaattgaagttgaggattagagagttcgttcaatatttgcttagactaggtgaaaaaaagactggatctagcgaaaccaggcaaacatttttaagtatagtgaaatcatgttactatgctgctcattgcccacctcatgtcgttgatagacacattagtagagtgattttcgagccagtaagtgccgcaaagtaaccgcgg

SEQ ID NO: 40 - protein, 827 aa, Zea mays
MVLSSSCTTVPHLSSLAVVQLGPWSSRIKKKTDTVAVPAAAGRWRRALARAQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 41 - DNA, 2570 bp, Artificial Sequence
  Codon-optimized CDPS
cttcttcactaaatacttagacagagaaaacagagctttttaaagccatgtctcttcagtatcatgttctaaactccattccaagtacaacctttctcagttctactaaaacaacaatatcttcttctttccttaccatctcaggatctcctctcaatgtcgctagagacaaatccagaagcggttccatacattgttcaaagcttcgaactcaagaatacattaattctcaagaggttcaacatgatttgcctctaatacatgagtggcaacagcttcaaggagaagatgctcctcagattagtgttggaagtaatagtaatgcattcaaagaagcagtgaagagtgtgaaaacgatcttgagaaacctaacggacggggaaattacgatatcggcttacgatacagcttgggttgcattgatcgatgccggagataaaactccggcgtttccctccgccgtgaaatggatcgccgagaaccaactttccgatggttcttggggagatgcgtatctcttctcttatcatgatcgtctcatcaatacccttgcatgcgtcgttgctctaagatcatggaatctctttcctcatcaatgcaacaaaggaatcacgtttttccgggaaaatattgggaagctagaagacgaaaatgatgagcatatgccaatcggattcgaagtagcattcccatcgttgcttgagatagctcgaggaataaacattgatgtaccgtacgattctccggtcttaaaagatatatacgccaagaaagagctaaagcttacaaggataccaaaagagataatgcacaagataccaacaacattgttgcatagtttggaggggatgcgtgatttagattgggaaaagctcttgaaacttcaatctcaagacggatctttcctcttctctccttcctctaccgcttttgcattcatgcagacccgagacagtaactgcctcgagtatttgcgaaatgccgtcaaacgtttcaatggaggagttcccaatgtctttcccgtggatcttttcgagcacatatggatagtggatcggttacaacgtttagggatatcgagatactttgaagaagagattaaagagtgtcttgactatgtccacagatattggaccgacaatggcatatgttgggctagatgttcccatgtccaagacatcgatgatacagccatggcatttaggctcttaagacaacatggataccaagtgtccgcagatgtattcaagaactttgagaaagagggagagtttttctgctttgtggggcaatcaaaccaagcagtaaccggtatgttcaacctataccgggcatcacaattggcgtttccaagggaagagatattgaaaaacgccaaagagttttcttataattatctgctagaaaaacgggagagagaggagttgattgataagtggattataatgaaagacttacctggcgagattgggtttgcgttagagattccatggtacgcaagcttgcctcgagtagagacgagattctatattgatcaatatggtggagaaaacgacgtttggattggcaagactctttataggatgccatacgtgaacaataatggatatctggaattagcaaaacaagattacaacaattgccaagctcagcatcagctcgaatgggacatattccaaaagtggtatgaagaaaataggttaagtgagtggggtgtgcgcagaagtgagcttctcgagtgttactacttagcggctgcaactatatttgaatcagaaaggtcacatgagagaatggtttgggctaagtcaagtgtattggttaaagccatttcttcttcttttggggaatcctctgactccagaagaagcttctccgatcagtttcatgaatacattgccaatgctcgacgaagtgatcatcactttaatgacaggaacatgagattggaccgaccaggatcggttcaggccagtcggcttgccggagtgttaatcgggactttgaatcaaatgtcttttgaccttttcatgtctcatggccgtgacgttaacaatctcctctatctatcgtggggagattggatggaaaaatggaaactatatggagatgaaggagaaggagagctcatggtgaagatgataattctaatgaagaacaatgacctaactaacttcttcacccacactcacttcgttcgtctcgcggaaatcatcaatcgaatctgtcttcctcgccaatacttaaaggcaaggagaaacgatgagaaggagaagacaataaagagtatggagaaggagatggggaaaatggttgagttagcattgtcggagagtgacacatttcgtgacgtcagcatcacgtttcttgatgtagcaaaagcattttactactttgctttatgtggcgatcatctccaaactcacatctccaaagtcttgtttcaaaaagtctagtaacctcatcatcatcatcgatccattaacaatcagtggatcgatgtatccatagatgcgtgaataatatttcatgtagagaaggagaacaaattagatcatgtagggttatca

SEQ ID NO: 42 - protein, 802 aa, Arabidopsis thaliana
MSLQYHVLNSIPSTTFLSSTKTTISSSFLTISGSPLNVARDKSRSGSIHCSKLRTQEYINSQEVQHDLPLIHEWQQLQGEDAPQISVGSNSNAFKEAVKSVKTILRNLTDGEITISAYDTAWVALIDAGDKTPAFPSAVKWIAENQLSDGSWGDAYLFSYHDRLINTLACVVALRSWNLFPHQCNKGITFFRENIGKLEDENDEHMPIGFEVAFPSLLEIARGINIDVPYDSPVLKDIYAKKELKLTRIPKEIMHKIPTTLLHSLEGMRDLDWEKLLKLQSQDGSFLFSPSSTAFAFMQTRDSNCLEYLRNAVKRFNGGVPNVFPVDLFEHIWIVDRLQRLGISRYFEEEIKECLDYVHRYWTDNGICWARCSHVQDIDDTAMAFRLLRQHGYQVSADVFKNFEKEGEFFCFVGQSNQAVTGMFNLYRASQLAFPREEILKNAKEFSYNYLLEKREREELIDKWIIMKDLPGEIGFALEIPWYASLPRVETRFYIDQYGGENDVWIGKTLYRMPYVNNNGYLELAKQDYNNCQAQHQLEWDIFQKWYEENRLSEWGVRRSELLECYYLAAATIFESERSHERMVWAKSSVLVKAISSSFGESSDSRRSFSDQFHEYIANARRSDHHFNDRNMRLDRPGSVQASRLAGVLIGTLNQMSFDLFMSHGRDVNNLLYLSWGDWMEKWKLYGDEGEGELMVKMIILMKNNDLTNFFTHTHFVRLAEIINRICLPRQYLKARRNDEKEKTIKSMEKEMGKMVELALSESDTFRDVSITFLDVAKAFYYFALCGDHLQTHISKVLFQKV

SEQ ID NO: 43 - DNA, 2355 bp, Artificial Sequence
  Codon-optimized KS
atgaatttgagtttgtgtatagcatctccactattgaccaaatctaatagaccagctgctttatcagcaattcatacagctagtacatcccatggtggccaaaccaaccctacgaatctgataatcgatacgaccaaggagagaatacaaaaacaattcaaaaatgttgaaatttcagtttcttcttatgatactgcgtgggttgccatggttccatcacctaattctccaaagtctccatgtttcccagaatgtttgaattggctgattaacaaccagttgaatgatggatcttggggtttagtcaatcacacgcacaatcacaaccatccacttttgaaagattctttatcctcaactttggcttgcatcgtggccctaaagagatggaacgtaggtgaggatcagattaacaaggggcttagtttcattgaatctaacttggcttccgcgactgaaaaatctcaaccatctccaataggattcgatatcatctttccaggtctgttagagtacgccaaaaatctagatatcaacttactgtctaagcaaactgatttctcactaatgttacacaagagagaattagaacaaaagagatgtcattcaaacgaaatggatggttacctagcttatatctctgaaggtcttggtaatctttacgattggaatatggtgaaaaagtaccagatgaaaaatggctcagttttcaattccccttctgcaactgcggcagcattcattaaccatcaaaatccaggatgcctgaactatttgaattcactactagacaaattcggcaacgcagttccaactgtataccctcacgatttgtttatcagattgagtatggtggatacaattgaaagacttggtatatcccaccactttagagtcgagatcaaaaatgttttggatgagacataccgttgttgggtggagagagatgaacaaatctttatggatgttgtgacgtgcgcgttggcctttagattgttgcgtattaacggttacgaagttagtccagatccacttgccgaaattacaaacgaattagctttaaaggatgaatacgccgctcttgaaacatatcatgcgtcacatatcctttaccaagaggacttatcatctggaaaacaaattcttaaatctgctgatttcctgaaggaaatcatatccactgatagtaatagactgtccaaactgatccataaagaggttgaaaatgcacttaagttccctattaacaccggcttagaacgtattaacacaagacgtaacatccagctttacaacgtagacaatactagaatcttgaaaaccacttaccattcttccaacatatcaaacactgattacctaagattagctgttgaagatttctacacatgtcagtctatctatagagaagagctgaaaggattagagagatgggtcgttgagaataagctagatcaattgaaatttgccagacaaaagacagcttattgttacttctcagttgccgccactttatcaagtccagaattgtcagatgcacgtatttcttgggctaaaaacggaattttgacaactgttgttgatgatttctttgatattggcgggacaatcgacgaattgacaaacctgattcaatgcgttgaaaagtggaatgtcgatgtcgataaagactgttgctcagaacatgttagaatactgttcttggctctgaaagatgctatctgttggatcggggatgaggctttcaaatggcaagctagagatgtgacgtctcacgtcattcaaacctggctagaactgatgaactctatgttgagagaagcaatttggactagagatgcatacgttcctacattaaacgagtatatggaaaacgcttatgtctcctttgctttgggtcctatcgttaagcctgccatatactttgtaggaccaaagctatccgaggaaatcgtcgaatcatcagaataccataacttgttcaagttaatgtccacacaaggcagattacttaatgatattcattctttcaaaagagagtttaaggaaggaaagttaaatgctgttgctctgcatctttctaatggcgaaagtggtaaagtcgaagaggaagtagttgaggaaatgatgatgatgatcaaaaacaagagaaaggagttgatgaaactaatcttcgaagagaacggttcaattgttcctagagcatgtaaggatgcattttggaacatgtgtcatgtgctaaactttttctacgcaaacgacgatggttttactgggaacacaatactagatacagtaaaagacatcatatacaaccctttggtcttagtaaacgaaaacgaggagcaaagataa

SEQ ID NO: 44 - protein, 784 aa, Stevia rebaudiana
MNLSLCIASPLLTKSNRPAALSAIHTASTSHGGQTNPTNLIIDTTKERIQKQFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATEKSQPSPIGFDIIFPGLLEYAKNLDINLLSKQTDFSLMLHKRELEQKRCHSNEMDGYLAYISEGLGNLYDWNMVKKYQMKNGSVFNSPSATAAAFINHQNPGCLNYLNSLLDKFGNAVPTVYPHDLFIRLSMVDTIERLGISHHFRVEIKNVLDETYRCWVERDEQIFMDVVTCALAFRLLRINGYEVSPDPLAEITNELALKDEYAALETYHASHILYQEDLSSGKQILKSADFLKEIISTDSNRLSKLIHKEVENALKFPINTGLERINTRRNIQLYNVDNTRILKTTYHSSNISNTDYLRLAVEDFYTCQSIYREELKGLERWVVENKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 45 - DNA, 2355 bp, Artificial Sequence
  Codon-optimized KS
atgaatctgtccctttgtatagctagtccactgttgacaaaatcttctagaccaactgctctttctgcaattcatactgccagtactagtcatggaggtcaaacaaacccaacaaatttgataatcgatactactaaggagagaatccaaaagctattcaaaaatgttgaaatctcagtatcatcttatgacaccgcatgggttgcaatggtgccatcacctaattccccaaaaagtccatgttttccagagtgcttgaattggttaatcaataatcagttaaacgatggttcttggggtttagtcaaccacactcataaccacaatcatccattattgaaggactctttatcatcaacattagcctgtattgttgcattgaaaagatggaatgtaggtgaagatcaaatcaacaagggtttatcattcatagaatccaatctagcttctgctaccgacaaatcacaaccatctccaatcgggttcgacataatcttccctggtttgctggagtatgccaaaaaccttgatatcaacttactgtctaaacaaacagatttctctttgatgctacacaaaagagagttagagcagaaaagatgccattctaacgaaattgacgggtacttagcatatatctcagaaggtttgggtaatttgtatgactggaacatggtcaaaaagtatcagatgaaaaatggatccgtattcaattctccttctgcaactgccgcagcattcattaatcatcaaaaccctgggtgtcttaactacttgaactcactattagataagtttggaaatgcagttccaacagtctatcctttggacttgtacatcagattatctatggttgacactatagagagattaggtatttctcatcatttcagagttgagatcaaaaatgttttggacgagacatacagatgttgggtcgaaagagatgagcaaatctttatggatgtcgtgacctgcgctctggcttttagattgctaaggatacacggatacaaagtatctcctgatcaactggctgagattacaaacgaactggctttcaaagacgaatacgccgcattagaaacataccatgcatcccaaatactttaccaggaagacctaagttcaggaaaacaaatcttgaagtctgcagatttcctgaaaggcattctgtctacagatagtaataggttgtctaaattgatacacaaggaagtagaaaacgcactaaagtttcctattaacactggtttagagagaatcaatactaggagaaacattcagctgtacaacgtagataatacaaggattcttaagaccacctaccatagttcaaacatttccaacacctattacttaagattagctgtcgaagacttttacacttgtcaatcaatctacagagaggagttaaagggcctagaaagatgggtagttcaaaacaagttggatcaactgaagtttgctagacagaagacagcatactgttatttctctgttgctgctaccctttcatccccagaattgtctgatgccagaataagttgggccaaaaatggtattcttacaactgtagtcgatgatttctttgatattggaggtactattgatgaactgacaaatcttattcaatgtgttgaaaagtggaacgtggatgtagataaggattgctgcagtgaacatgtgagaatacttttcctggctctaaaagatgcaatatgttggattggcgacgaggccttcaagtggcaagctagagatgttacatctcatgtcatccaaacttggcttgaactgatgaactcaatgctaagagaagcaatctggacaagagatgcatacgttccaacattgaacgaatacatggaaaacgcttacgtctcatttgccttgggtcctattgttaagccagccatatactttgttgggccaaagttatccgaagagattgttgagtcttccgaatatcataacctattcaagttaatgtcaacacaaggcagacttctgaacgatatccactccttcaaaagagaattcaaggaaggtaagctaaacgctgttgctttgcacttgtctaatggtgaatctggcaaagtggaagaggaagtcgttgaggaaatgatgatgatgatcaaaaacaagagaaaggaattgatgaaattgattttcgaggaaaatggttcaatcgtacctagagcttgtaaagatgctttttggaatatgtgccatgttcttaacttcttttacgctaatgatgatggcttcactggaaatacaatattggatacagttaaagatatcatctacaacccacttgttttggtcaatgagaacgaggaacaaagataa

SEQ ID NO: 46 - protein, 784 aa, Stevia rebaudiana
MNLSLCIASPLLTKSSRPTALSAIHTASTSHGGQTNPTNLIIDTTKERIQKLFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATDKSQPSPIGFDIIFPGLLEYAKNLDINLLSKQTDFSLMLHKRELEQKRCHSNEIDGYLAYISEGLGNLYDWNMVKKYQMKNGSVFNSPSATAAAFINHQNPGCLNYLNSLLDKFGNAVPTVYPLDLYIRLSMVDTIERLGISHHFRVEIKNVLDETYRCWVERDEQIFMDVVTCALAFRLLRIHGYKVSPDQLAEITNELAFKDEYAALETYHASQILYQEDLSSGKQILKSADFLKGILSTDSNRLSKLIHKEVENALKFPINTGLERINTRRNIQLYNVDNTRILKTTYHSSNISNTYYLRLAVEDFYTCQSIYREELKGLERWVVQNKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 47 - DNA, 1773 bp, Artificial Sequence
  Codon-optimized KS
atggctatgccagtgaagctaacacctgcgtcattatccttaaaagctgtgtgctgcagattctcatccggtggccatgctttgagattcgggagtagtctgccatgttggagaaggacccctacccaaagatctacttcttcctctactactagaccagctgccgaagtgtcatcaggtaagagtaaacaacatgatcaggaagctagtgaagcgactatcagacaacaattacaacttgtggatgtcctggagaatatgggaatatccagacattttgctgcagagataaagtgcatactagacagaacttacagatcttggttacaaagacacgaggaaatcatgctggacactatgacatgtgctatggcttttagaatcctaagattgaacggatacaacgtttcatcagatgaactataccacgttgtagaggcatctggtctgcataattctttgggtgggtatcttaacgataccagaacactacttgaattacacaaggcttcaacagttagtatctctgaggatgaatctatcttagattcaattggctctagatccagaacattgcttagagaacaattggagtctggtggcgcactgagaaagccttctttattcaaagaggttgaacatgcactggatggacctttttacaccacacttgatagacttcatcataggtggaatattgaaaacttcaacattattgagcaacacatgttggagactccatacttatctaaccagcatacatcaagggatatcctagcattgtcaattagagatttttcctcctcacaattcacttatcaacaagagctacagcatctggagagttgggttaaggaatgtagattagatcaactacagttcgcaagacagaaattagcgtacttttacctatcagccgcaggcaccatgttttctcctgagctttctgatgcgagaacattatgggccaaaaacggggtgttgacaactattgttgatgatttctttgatgttgccggttctaaagaggaattggaaaacttagtcatgctggtcgaaatgtgggatgaacatcacaaagttgaattctattctgagcaggtcgaaatcatcttctcttccatctacgattctgtcaaccaattgggtgagaaggcctctttggttcaagacagatcaattacaaaacaccttgttgaaatatggttagacttgttaaagtccatgatgacggaagttgaatggagactgtcaaaatacgtgcctacagaaaaggaatacatgattaatgcctctcttatcttcggcctaggtccaatcgttttaccagctttgtatttcgttggtccaaagatttcagaaagtatagtaaaggacccagaatatgatgaattgttcaaactaatgtcaacatgtggtagattgttgaatgacgtgcaaacgttcgaaagagaatacaatgagggtaaactgaattctgtcagtctattggttcttcacggaggcccaatgtctatttcagacgcaaagaggaaattacaaaagcctattgatacgtgtagaagagatcttctttctttggtccttagagaagagtctgtagtaccaagaccatgtaaggaactattctggaaaatgtgtaaagtgtgctatttcttttactcaacaactgatgggttttctagtcaagtcgaaagagcaaaagaggtagacgctgtcataaatgagccactgaagttgcaaggttctcatacactggtatctgatgtttaa

SEQ ID NO: 48 - protein, 590 aa, Zea mays
MAMPVKLTPASLSLKAVCCRFSSGGHALRFGSSLPCWRRTPTQRSTSSSTTRPAAEVSSGKSKQHDQEASEATIRQQLQLVDVLENMGISRHFAAEIKCILDRTYRSWLQRHEEIMLDTMTCAMAFRILRLNGYNVSSDELYHVVEASGLHNSLGGYLNDTRTLLELHKASTVSISEDESILDSIGSRSRTLLREQLESGGALRKPSLFKEVEHALDGPFYTTLDRLHHRWNIENFNIIEQHMLETPYLSNQHTSRDILALSIRDFSSSQFTYQQELQHLESWVKECRLDQLQFARQKLAYFYLSAAGTMFSPELSDARTLWAKNGVLTTIVDDFFDVAGSKEELENLVMLVEMWDEHHKVEFYSEQVEIIFSSIYDSVNQLGEKASLVQDRSITKHLVEIWLDLLKSMMTEVEWRLSKYVPTEKEYMINASLIFGLGPIVLPALYFVGPKISESIVKDPEYDELFKLMSTCGRLLNDVQTFEREYNEGKLNSVSLLVLHGGPMSISDAKRKLQKPIDTCRRDLLSLVLREESVVPRPCKELFWKMCKVCYFFYSTTDGFSSQVERAKEVDAVINEPLKLQGSHTLVSDV

SEQ ID NO: 49 - DNA, 2232 bp, Artificial Sequence
  Codon-optimized KS
atgcagaacttccatggtacaaaggaaaggatcaaaaagatgtttgacaagattgaattgtccgtttcttcttatgatacagcctgggttgcaatggtcccatcccctgattgcccagaaacaccttgttttccagaatgtactaaatggatcctagaaaatcagttgggtgatggtagttggtcacttcctcatggcaatccacttctagttaaagatgcattatcttccactcttgcttgtattctggctcttaaaagatggggaatcggtgaggaacagattaacaaaggactgagattcatagaactcaactctgctagtgtaaccgataacgaacaacacaaaccaattggatttgacattatctttccaggtatgattgaatacgctatagacttagacctgaatctaccactaaaaccaactgacattaactccatgttgcatcgtagagcccttgaattgacatcaggtggaggcaaaaatctagaaggtagaagagcttacttggcctacgtctctgaaggaatcggtaagctgcaagattgggaaatggctatgaaataccaacgtaaaaacggatctctgttcaatagtccatcaacaactgcagctgcattcatccatatacaagatgctgaatgcctccactatattcgttctcttctccagaaatttggaaacgcagtccctacaatataccctctcgatatctatgccagactttcaatggtagatgccctggaacgtcttggtattgatagacatttcagaaaggagagaaagttcgttctggatgaaacatacagattttggttgcaaggagaagaggagattttctccgataacgcaacctgtgctttggccttcagaatattgagacttaatggttacgatgtctctcttgaagatcacttctctaactctctgggcggttacttaaaggactcaggagcagctttagaactgtacagagccctccaattgtcttacccagacgagtccctcctggaaaagcaaaattctagaacttcttacttcttaaaacaaggtttatccaatgtctccctctgtggtgacagattgcgtaaaaacataattggagaggtgcatgatgctttaaacttttccgaccacgctaacttacaaagattagctattcgtagaaggattaagcattacgctactgacgatacaaggattctaaaaacttcctacagatgctcaacaatcggtaaccaagattttctaaaacttgcagtggaagatttcaatatctgtcaatcaatacaaagagaggaattcaagcatattgaaagatgggtcgttgaaagacgtctagacaagttaaagttcgctagacaaaaagaggcctattgctatttctcagccgcagcaacattgtttgcccctgaattgtctgatgctagaatgtcttgggccaaaaatggtgtattgacaactgtggttgatgatttcttcgatgtcggaggctctgaagaggaattagttaacttgatagaattgatcgagcgttgggatgtgaatggcagtgcagatttttgtagtgaggaagttgagattatctattctgctatccactcaactatctctgaaataggtgataagtcatttggctggcaaggtagagatgtaaagtctcaagttatcaagatctggctggacttattgaaatcaatgttaactgaagctcaatggtcttcaaacaagtctgttcctaccctagatgagtatatgacaaccgcccatgtttcattcgcacttggtccaattgtacttccagccttatacttcgttggcccaaagttgtcagaagaggttgcaggtcatcctgaactactaaacctctacaaagtcacatctacttgtggcagactactgaatgattggagaagttttaagagagaatccgaggaaggtaagctcaacgctattagtttatacatgatccactccggtggtgcttctacagaagaggaaacaatcgaacatttcaaaggtttgattgattctcagagaaggcaactgttacaattggtgttgcaagagaaggatagtatcatacctagaccatgtaaagatctattttggaatatgattaagttattacacactttctacatgaaagatgatggcttcacctcaaatgagatgaggaatgtagttaaggcaatcattaacgaaccaatctcactggatgaattatga

SEQ ID NO: 50 - protein, 775 aa, Populus trichocarpa
MSCIRPWFCPSSISATLTDPASKLVTGEFKTTSLNFHGTKERIKKMFDKIELSVSSYDTAWVAMVPSPDCPETPCFPECTKWILENQLGDGSWSLPHGNPLLVKDALSSTLACILALKRWGIGEEQINKGLRFIELNSASVTDNEQHKPIGFDIIFPGMIEYAKDLDLNLPLKPTDINSMLHRRALELTSGGGKNLEGRRAYLAYVSEGIGKLQDWEMAMKYQRKNGSLFNSPSTTAAAFIHIQDAECLHYIRSLLQKFGNAVPTIYPLDIYARLSMVDALERLGIDRHFRKERKFVLDETYRFWLQGEEEIFSDNATCALAFRILRLNGYDVSLEDHFSNSLGGYLKDSGAALELYRALQLSYPDESLLEKQNSRTSYFLKQGLSNVSLCGDRLRKNIIGEVHDALNFPDHANLQRLAIRRRIKHYATDDTRILKTSYRCSTIGNQDFLKLAVEDFNICQSIQREEFKHIERWVVERRLDKLKFARQKEAYCYFSAAATLFAPELSDARMSWAKNGVLTTVVDDFFDVGGSEEELVNLIELIERWDVNGSADFCSEEVEIIYSAIHSTISEIGDKSFGWQGRDVKSHVIKIWLDLLKSMLTEAQWSSNKSVPTLDEYMTTAHVSFALGPIVLPALYFVGPKLSEEVAGHPELLNLYKVMSTCGRLLNDWRSFKRESEEGKLNAISLYMIHSGGASTEEETIEHFKGLIDSQRRQLLQLVLQEKDSIIPRPCKDLFWNMIKLLHTFYMKDDGFTSNEMRNVVKAIINEPISLDEL

SEQ ID NO: 51 - DNA, 2358 bp, Artificial Sequence
  Codon-optimized KS
atgtctatcaaccttcgctcctccggttgttcgtctccgatctcagctactttggaacgaggattggactcagaagtacagacaagagctaacaatgtgagctttgagcaaacaaaggagaagattaggaagatgttggagaaagtggagctttctgtttcggcctacgatactagttgggtagcaatggttccatcaccgagctcccaaaatgctccacttttcccacagtgtgtgaaatggttattggataatcaacatgaagatggatcttggggacttgataaccatgaccatcaatctcttaagaaggatgtgttatcatctacactggctagtatcctcgcgttaaagaagtggggaattggtgaaagacaaataaacaagggtctccagtttattgagctgaattctgcattagtcactgatgaaaccatacagaaaccaacagggtttgatattatatttcctgggatgattaaatatgctagagatttgaatctgacgattccattgggctcagaagtggtggatgacatgatacgaaaaagagatctggatcttaaatgtgatagtgaaaagttttcaaagggaagagaagcatatctggcctatgttttagaggggacaagaaacctaaaagattgggatttgatagtcaaatatcaaaggaaaaatgggtcactgtttgattctccagccacaacagcagctgcttttactcagtttgggaatgatggttgtctccgttatctctgttctctccttcagaaattcgaggctgcagttccttcagtttatccatttgatcaatatgcacgccttagtataattgtcactcttgaaagcttaggaattgatagagatttcaaaaccgaaatcaaaagcatattggatgaaacctatagatattggcttcgtggggatgaagaaatatgtttggacttggccacttgtgctttggctttccgattattgcttgctcatggctatgatgtgtcttacgatccgctaaaaccatttgcagaagaatctggtttctctgatactttggaaggatatgttaagaatacgttttctgtgttagaattatttaaggctgctcaaagttatccacatgaatcagctttgaagaagcagtgttgttggactaaacaatatctggagatggaattgtccagctgggttaagacctctgttcgagataaatacctcaagaaagaggtcgaggatgctcttgcttttccctcctatgcaagcctagaaagatcagatcacaggagaaaaatactcaatggttctgctgtggaaaacaccagagttacaaaaacctcatatcgtttgcacaatatttgcacctctgatatcctgaagttagctgtggatgacttcaatttctgccagtccatacaccgtgaagaaatggaacgtcttgataggtggattgtggagaatagattgcaggaactgaaatttgccagacagaagctggcttactgttatttctctggggctgcaactttattttctccagaactatctgatgctcgtatatcgtgggccaaaggtggagtacttacaacggttgtagacgacttctttgatgttggagggtccaaagaagaactggaaaacctcatacacttggtcgaaaagtgggatttgaacggtgttcctgagtacagctcagaacatgttgagatcatattctcagttctaagggacaccattctcgaaacaggagacaaagcattcacctatcaaggacgcaatgtgacacaccacattgtgaaaatttggttggatctgctcaagtctatgttgagagaagccgagtggtccagtgacaagtcaacaccaagcttggaggattacatggaaaatgcgtacatatcatttgcattaggaccaattgtcctcccagctacctatctgatcggacctccacttccagagaagacagtcgatagccaccaatataatcagctctacaagctcgtgagcactatgggtcgtcttctaaatgacatacaaggttttaagagagaaagcgcggaagggaagctgaatgcggtttcattgcacatgaaacacgagagagacaatcgcagcaaagaagtgatcatagaatcgatgaaaggtttagcagagagaaagagggaagaattgcataagctagttttggaggagaaaggaagtgtggttccaagggaatgcaaagaagcgttcttgaaaatgagcaaagtgttgaacttattttacaggaaggacgatggattcacatcaaatgatctgatgagtcttgttaaatcagtgatctacgagcctgttagcttacagaaagaatctttaacttga

SEQ ID NO: 52 - protein, 785 aa, Arabidopsis thaliana
MSINLRSSGCSSPISATLERGLDSEVQTRANNVSFEQTKEKIRKMLEKVELSVSAYDTSWVAMVPSPSSQNAPLFPQCVKWLLDNQHEDGSWGLDNHDHQSLKKDVLSSTLASILALKKWGIGERQINKGLQFIELNSALVTDETIQKPTGFDIIFPGMIKYARDLNLTIPLGSEVVDDMIRKRDLDLKCDSEKFSKGREAYLAYVLEGTRNLKDWDLIVKYQRKNGSLFDSPATTAAAFTQFGNDGCLRYLCSLLQKFEAAVPSVYPFDQYARLSIIVTLESLGIDRDFKTEIKSILDETYRYWLRGDEEICLDLATCALAFRLLLAHGYDVSYDPLKPFAEESGFSDTLEGYVKNTFSVLELFKAAQSYPHESALKKQCCWTKQYLEMELSSWVKTSVRDKYLKKEVEDALAFPSYASLERSDHRRKILNGSAVENTRVTKTSYRLHNICTSDILKLAVDDFNFCQSIHREEMERLDRWIVENRLQELKFARQKLAYCYFSGAATLFSPELSDARISWAKGGVLTTVVDDFFDVGGSKEELENLIHLVEKWDLNGVPEYSSEHVEIIFSVLRDTILETGDKAFTYQGRNVTHHIVKIWLDLLKSMLREAEWSSDKSTPSLEDYMENAYISFALGPIVLPATYLIGPPLPEKTVDSHQYNQLYKLVSTMGRLLNDIQGFKRESAEGKLNAVSLHMKHERDNRSKEVIIESMKGLAERKREELHKLVLEEKGSVVPRECKEAFLKMSKVLNLFYRKDDGFTSNDLMSLVKSVIYEPVSLQKESLT

SEQ ID NO: 53 - DNA, 2952 bp, Artificial Sequence
  Codon-optimized CDPS-KS
atggaatttgatgaaccattggttgacgaagcaagatctttagtgcagcgtactttacaagattatgatgacagatacggcttcggtactatgtcatgtgctgcttatgatacagcctgggtgtctttagttacaaaaacagtcgatgggagaaaacaatggcttttcccagagtgttttgaatttctactagaaacacaatctgatgccggaggatgggaaatcgggaattcagcaccaatcgacggtatattgaatacagctgcatccttacttgctctaaaacgtcacgttcaaactgagcaaatcatccaacctcaacatgaccataaggatctagcaggtagagctgaacgtgccgctgcatctttgagagcacaattggctgcattggatgtgtctacaactgaacacgtcggttttgagataattgttcctgcaatgctagacccattagaagccgaagatccatctctagttttcgattttccagctaggaaacctttgatgaagattcatgatgctaagatgagtagattcaggccagaatacttgtatggcaaacaaccaatgaccgccttacattcattagaggctttcataggcaaaatcgacttcgataaggtaagacaccaccgtacccatgggtctatgatgggttctccttcatctaccgcagcctacttaatgcacgcttcacaatgggatggtgactcagaggcttaccttagacacgtgattaaacacgcagcagggcagggaactggtgctgtaccatctgctttcccatcaacacattttgagtcatcttggattcttaccacattgtttagagctggattttcagcttctcatcttgcctgtgatgagttgaacaagttggtcgagatacttgagggctcattcgagaaggaaggtggggcaatcggttacgctccagggtttcaagcagatgttgatgatactgctaaaacaataagtacattagcagtccttggaagagatgctacaccaagacaaatgatcaaggtatttgaagctaatacacattttagaacataccctggtgaaagagatccttctttgacagctaattgtaatgctctatcagccttactacaccaaccagatgcagcaatgtatggatctcaaattcaaaagattaccaaatttgtctgtgactattggtggaagtctgatggtaagattaaagataagtggaacacttgctacttgtacccatctgtcttattagttgaggttttggttgatcttgttagtttattggagcagggtaaattgcctgatgttttggatcaagagcttcaatacagagtcgccatcacattgttccaagcatgtttaaggccattactagaccaagatgccgaaggatcatggaacaagtctatcgaagccacagcctacggcatccttatcctaactgaagctaggagagtttgtttcttcgacagattgtctgagccattgaatgaggcaatccgtagaggtatcgctttcgccgactctatgtctggaactgaagctcagttgaactacatttggatcgaaaaggttagttacgcacctgcattattgactaaatcctatttgttagcagcaagatgggctgctaagtctcctttaggcgcttccgtaggctcttctttgtggactccaccaagagaaggattggataagcatgtcagattattccatcaagctgagttattcagatcccttccagaatgggaattaagagcctccatgattgaagcagctttgttcacaccacttctaagagcacatagactagacgttttccctagacaagatgtaggtgaagacaaatatcttgatgtagttccattcttttggactgccgctaacaacagagatagaacttacgcttccactctattcctttacgatatgtgttttatcgcaatgttaaacttccagttagacgaattcatggaggccacagccggtatcttattcagagatcatatggatgatttgaggcaattgattcatgatcttttggcagagaaaacttccccaaagagttctggtagaagtagtcagggcacaaaagatgctgactcaggtatagaggaagacgtgtcaatgtccgattcagcttcagattcccaggatagaagtccagaatacgacttggttttcagtgcattgagtacctttacaaaacatgtcttgcaacacccatctatacaaagtgcctctgtatgggatagaaaactacttgctagagagatgaaggcttacttacttgctcatatccaacaagcagaagattcaactccattgtctgaattgaaagatgtgcctcaaaagactgatgtaacaagagtttctacatctactactaccttctttaactgggttagaacaacttccgcagaccatatatcctgcccatactccttccactttgtagcatgccatctaggcgcagcattgtcacctaaagggtctaacggtgattgctatccttcagctggtgagaagttcttggcagctgcagtctgcagacatttggccaccatgtgtagaatgtacaacgatcttggatcagctgaacgtgattctgatgaaggtaatttgaactccttggacttccctgaattcgccgattccgcaggaaacggagggatagaaattcagaaggccgctctattaaggttagctgagtttgagagagattcatacttagaggccttccgtcgtttacaagatgaatccaatagagttcacggtccagccggtggtgatgaagccagattgtccagaaggagaatggcaatccttgaattcttcgcccagcaggtagatttgtacggtcaagtatacgtcattagggatatttccgctcgtattcctaaaaacgaggttgagaaaaagagaaaattggatgatgctttcaattga

SEQ ID NO: 54 - protein, 983 aa, Phomopsis amygdali
MEFDEPLVDEARSLVQRTLQDYDDRYGFGTMSCAAYDTAWVSLVTKTVDGRKQWLFPECFEFLLETQSDAGGWEIGNSAPIDGILNTAASLLALKRHVQTEQIIQPQHDHKDLAGRAERAAASLRAQLAALDVSTTEHVGFEIIVPAMLDPLEAEDPSLVFDFPARKPLMKIHDAKMSRFRPEYLYGKQPMTALHSLEAFIGKIDFDKVRHHRTHGSMMGSPSSTAAYLMHASQWDGDSEAYLRHVIKHAAGQGTGAVPSAFPSTHFESSWILTTLFRAGFSASHLACDELNKLVEILEGSFEKEGGAIGYAPGFQADVDDTAKTISTLAVLGRDATPRQMIKVFEANTHFRTYPGERDPSLTANCNALSALLHQPDAAMYGSQIQKITKFVCDYWWKSDGKIKDKWNTCYLYPSVLLVEVLVDLVSLLEQGKLPDVLDQELQYRVAITLFQACLRPLLDQDAEGSWNKSIEATAYGILILTEARRVCFFDRLSEPLNEAIRRGIAFADSMSGTEAQLNYIWIEKVSYAPALLTKSYLLAARWAAKSPLGASVGSSLWTPPREGLDKHVRLFHQAELFRSLPEWELRASMIEAALFTPLLRAHRLDVFPRQDVGEDKYLDVVPFFWTAANNRDRTYASTLFLYDMCFIAMLNFQLDEFMEATAGILFRDHMDDLRQLIHDLLAEKTSPKSSGRSSQGTKDADSGIEEDVSMSDSASDSQDRSPEYDLVFSALSTFTKHVLQHPSIQSASVWDRKLLAREMKAYLLAHIQQAEDSTPLSELKDVPQKTDVTRVSTSTTTFFNWVRTTSADHISCPYSFHFVACHLGAALSPKGSNGDCYPSAGEKFLAAAVCRHLATMCRMYNDLGSAERDSDEGNLNSLDFPEFADSAGNGGIEIQKAALLRLAEFERDSYLEAFRRLQDESNRVHGPAGGDEARLSRRRMAILEFFAQQVDLYGQVYVIRDISARIPKNEVEKKRKLDDAFN

SEQ ID NO: 55 - DNA, 2646 bp, Artificial Sequence
  Codon-optimized CDPS-KS
atggcttctagtacacttatccaaaacagatcatgtggcgtcacatcatctatgtcaagttttcaaatcttcagaggtcaaccactaagatttcctggcactagaaccccagctgcagttcaatgcttgaaaaagaggagatgccttaggccaaccgaatccgtactagaatcatctcctggctctggttcatatagaatagtaactggcccttctggaattaaccctagttctaacgggcacttgcaagagggttccttgactcacaggttaccaataccaatggaaaaatctatcgataacttccaatctactctatatgtgtcagatatttggtctgaaacactacagagaactgaatgtttgctacaagtaactgaaaacgtccagatgaatgagtggattgaggaaattagaatgtactttagaaatatgactttaggtgaaatttccatgtccccttacgacactgcttgggtggctagagttccagcgttggacggttctcatgggcctcaattccacagatctttgcaatggattatcgacaaccaattaccagatggggactggggcgaaccttctcttttcttgggttacgatagagtttgtaatactttagcctgtgtgattgcgttgaaaacatggggtgttggggcacaaaacgttgaaagaggaattcagttcctacaatctaacatatacaagatggaggaagatgacgctaatcatatgccaataggattcgaaatcgtattccctgctatgatggaagatgccaaagcattaggtttggatttgccatacgatgctactattttgcaacagatttcagccgaaagagagaaaaagatgaaaaagatcccaatggcaatggtgtacaaatacccaaccactttacttcactccttagaaggcttgcatagagaagttgattggaataagttgttacaattacaatctgaaaatggtagttttctttattcacctgcttcaaccgcatgcgccttaatgtacactaaggacgttaaatgttttgattacttaaaccagttgttgatcaagttcgaccacgcatgcccaaatgtatatccagtcgatctattcgaaagattatggatggttgacagattgcagagattagggatctccagatactttgaaagagagattagagattgtttacaatacgtctacagatattggaaagattgtggaatcggatgggcttctaactcttccgtacaagatgttgatgatacagccatggcgtttagacttttaaggactcatggtttcgacgtaaaggaagattgctttagacagtttttcaaggacggagaattcttctgcttcgcaggccaatcatctcaagcagttacaggcatgtttaatctttcaagagccagtcaaacattgtttccaggagaatctttattgaaaaaggctagaaccttctctagaaacttcttgagaacaaagcatgagaacaacgaatgtttcgataaatggatcattactaaagatttggctggtgaagtcgagtataacttgaccttcccatggtatgcctctttgcctagattagaacataggacatacttagatcaatatggaatcgatgatatctggataggcaaatctttatacaaaatgcctgctgttaccaacgaagttttcctaaagttggcaaaggcagactttaacatgtgtcaagctctacacaaaaaggaattggaacaagtgataaagtggaacgcgtcctgtcaattcagagatcttgaattcgccagacaaaaatcagtagaatgctattttgctggtgcagccacaatgttcgaaccagaaatggttcaagctagattagtctgggcaagatgttgtgtattgacaactgtcttagacgattactttgaccacgggacacctgttgaggaacttagagtgtttgttcaagctgtcagaacatggaatccagagttgatcaacggtttgccagagcaagctaaaatcttgtttatgggcttatacaaaacagttaacacaattgcagaggaagcattcatggcacagaaaagagacgtccatcatcatttgaaacactattgggacaagttgataacaagtgccctaaaggaggccgaatgggcagagtcaggttacgtcccaacatttgatgaatacatggaagtagctgaaatttctgttgctctagaaccaattgtctgtagtaccttgttctttgcgggtcatagactagatgaggatgttctagatagttacgattaccatctagttatgcatttggtaaacagagtcggtagaatcttgaatgatatacaaggcatgaagagggaggcttcacaaggtaagatctcatcagttcaaatctacatggaggaacatccatctgttccatctgaggccatggcgatcgctcatcttcaagagttagttgataattcaatgcagcaattgacatacgaagttcttaggttcactgcggttccaaaaagttgtaagagaatccacttgaatatggctaaaatcatgcatgccttctacaaggatactgatggattctcatcccttactgcaatgacaggattcgtcaaaaaggttcttttcgaacctgtgcctgagtaa

SEQ ID NO: 56 - protein, 881 aa, Physcomitrella patens
MASSTLIQNRSCGVTSSMSSFQIFRGQPLRFPGTRTPAAVQCLKKRRCLRPTESVLESSPGSGSYRIVTGPSGINPSSNGHLQEGSLTHRLPIPMEKSIDNFQSTLYVSDIWSETLQRTECLLQVTENVQMNEWIEEIRMYFRNMTLGEISMSPYDTAWVARVPALDGSHGPQFHRSLQWIIDNQLPDGDWGEPSLFLGYDRVCNTLACVIALKTWGVGAQNVERGIQFLQSNIYKMEEDDANHMPIGFEIVFPAMMEDAKALGLDLPYDATILQQISAEREKKMKKIPMAMVYKYPTTLLHSLEGLHREVDWNKLLQLQSENGSFLYSPASTACALMYTKDVKCFDYLNQLLIKFDHACPNVYPVDLFERLWMVDRLQRLGISRYFEREIRDCLQYVYRYWKDCGIGWASNSSVQDVDDTAMAFRLLRTHGFDVKEDCFRQFFKDGEFFCFAGQSSQAVTGMFNLSRASQTLFPGESLLKKARTFSRNFLRTKHENNECFDKWIITKDLAGEVEYNLTFPWYASLPRLEHRTYLDQYGIDDIWIGKSLYKMPAVTNEVFLKLAKADFNMCQALHKKELEQVIKWNASCQFRDLEFARQKSVECYFAGAATMFEPEMVQARLVWARCCVLTTVLDDYFDHGTPVEELRVFVQAVRTWNPELINGLPEQAKILFMGLYKTVNTIAEEAFMAQKRDVHHHLKHYWDKLITSALKEAEWAESGYVPTFDEYMEVAEISVALEPIVCSTLFFAGHRLDEDVLDSYDYHLVMHLVNRVGRILNDIQGMKREASQGKISSVQIYMEEHPSVPSEAMAIAHLQELVDNSMQQLTYEVLRFTAVPKSCKRIHLNMAKIMHAFYKDTDGFSSLTAMTGFVKKVLFEPVPE

SEQ ID NO: 57 - DNA, 2859 bp, Artificial Sequence
  Codon-optimized CDPS-KS
atgcctggtaaaattgaaaatggtaccccaaaggacctcaagactggaaatgattttgtttctgctgctaagagtttactagatcgagctttcaaaagtcatcattcctactacggattatgctcaacttcatgtcaagtttatgatacagcttgggttgcaatgattccaaaaacaagagataatgtaaaacagtggttgtttccagaatgtttccattacctcttaaaaacacaagccgcagatggctcatggggttcattgcctacaacacagacagcgggtatcctagatacagcctcagctgtgctggcattattgtgccacgcacaagagcctttacaaatattggatgtatctccagatgaaatggggttgagaatagaacacggtgtcacatccttgaaacgtcaattagcagtttggaatgatgtggaggacaccaaccatattggcgtcgagtttatcataccagccttactttccatgctagaaaaggaattagatgttccatcttttgaatttccatgtaggtccatcttagagagaatgcacggggagaaattaggtcatttcgacctggaacaagtttacggcaagccaagctcattgttgcactcattggaagcatttctcggtaagctagattttgatcgactatcacatcacctataccacggcagtatgatggcatctccatcttcaacggctgcttatcttattggggctacaaaatgggatgacgaagccgaagattacctaagacatgtaatgcgtaatggtgcaggacatgggaatggaggtatttctggtacatttccaactactcatttcgaatgtagctggattatagcaacgttgttaaaggttggctttactttgaagcaaattgacggcgatggcttaagaggtttatcaaccatcttacttgaggcgcttcgtgatgagaatggtgtcataggctttgcccctagaacagcagatgtagatgacacagccaaagctctattggccttgtcattggtaaaccagccagtgtcacctgatatcatgattaaggtctttgagggcaaagaccattttaccacttttggttcagaaagagatccatcattgacttccaacctgcacgtccttttatctttacttaaacaatctaacttgtctcaataccatcctcaaatcctcaaaacaacattattcacttgtagatggtggtggggttccgatcattgtgtcaaagacaaatggaatttgagtcacctatatccaactatgttgttggttgaagccttcactgaagtgctccatctcattgacggtggtgaattgtctagtctgtttgatgaatcctttaagtgtaagattggtcttagcatctttcaagcggtacttagaataatcctcacccaagacaacgacggctcttggagaggatacagagaacagacgtgttacgcaatattggctttagttcaagcgagacatgtatgctttttcactcacatggttgacagactgcaatcatgtgttgatcgaggtttctcatggttgaaatcttgctcttttcattctcaagacctgacttggacctctaaaacagcttatgaagtgggtttcgtagctgaagcatataaactagctgctttacaatctgcttccctggaggttcctgctgccaccattggacattctgtcacgtctgccgttccatcaagtgatcttgaaaaatacatgagattggtgagaaaaactgcgttattctctccactggatgagtggggtctaatggcttctatcatcgaatcttcatttttcgtaccattactgcaggcacaaagagttgaaatataccctagagataatatcaaggtggacgaagataagtacttgtctattatcccattcacatgggtcggatgcaataataggtctagaactttcgcaagtaacagatggctatacgatatgatgtacctttcattactcggctatcaaaccgacgagtacatggaagctgtagctgggccagtgtttggggatgtttccttgttacatcaaacaattgataaggtgattgataatacaatgggtaaccttgcgagagccaatggaacagtacacagtggtaatggacatcagcacgaatctcctaatataggtcaagtcgaggacaccttgactcgtttcacaaattcagtcttgaatcacaaagacgtccttaactctagctcatctgatcaagatactttgagaagagagtttagaacattcatgcacgctcatataacacaaatcgaagataactcacgattcagtaagcaagcctcatccgatgcgttttcctctcctgaacaatcttactttcaatgggtgaactcaactggtggctcacatgtcgcttgcgcctattcatttgccttctctaattgcctcatgtctgcaaatttgttgcagggtaaagacgcatttccaagcggaacgcaaaagtacttaatctcctctgttatgagacatgccacaaacatgtgtagaatgtataacgactttggctctattgccagagacaacgctgagagaaatgttaatagtattcattttcctgagtttactctctgtaacggaacttctcaaaacctagatgaaaggaaggaaagacttctgaaaatcgcaacttacgaacaagggtatttggatagagcactagaggccttggaaagacagagtagagatgatgccggagacagagctggatctaaagatatgagaaagttgaaaatcgttaagttattctgtgatgttacggacttatacgatcagctctacgttatcaaagatttgtcatcctctatgaagtaa

SEQ ID NO: 58 - protein, 952 aa, Gibberella fujikuroi
MPGKIENGTPKDLKTGNDFVSAAKSLLDRAFKSHHSYYGLCSTSCQVYDTAWVAMIPKTRDNVKQWLFPECFHYLLKTQAADGSWGSLPTTQTAGILDTASAVLALLCHAQEPLQILDVSPDEMGLRIEHGVTSLKRQLAVWNDVEDTNHIGVEFIIPALLSMLEKELDVPSFEFPCRSILERMHGEKLGHFDLEQVYGKPSSLLHSLEAFLGKLDFDRLSHHLYHGSMMASPSSTAAYLIGATKWDDEAEDYLRHVMRNGAGHGNGGISGTFPTTHFECSWIIATLLKVGFTLKQIDGDGLRGLSTILLEALRDENGVIGFAPRTADVDDTAKALLALSLVNQPVSPDIMIKVFEGKDHFTTFGSERDPSLTSNLHVLLSLLKQSNLSQYHPQILKTTLFTCRWWWGSDHCVKDKWNLSHLYPTMLLVEAFTEVLHLIDGGELSSLFDESFKCKIGLSIFQAVLRIILTQDNDGSWRGYREQTCYAILALVQARHVCFFTHMVDRLQSCVDRGFSWLKSCSFHSQDLTWTSKTAYEVGFVAEAYKLAALQSASLEVPAATIGHSVTSAVPSSDLEKYMRLVRKTALFSPLDEWGLMASIIESSFFVPLLQAQRVEIYPRDNIKVDEDKYLSIIPFTWVGCNNRSRTFASNRWLYDMMYLSLLGYQTDEYMEAVAGPVFGDVSLLHQTIDKVIDNTMGNLARANGTVHSGNGHQHESPNIGQVEDTLTRFTNSVLNHKDVLNSSSSDQDTLRREFRTFMHAHITQIEDNSRFSKQASSDAFSSPEQSYFQWVNSTGGSHVACAYSFAFSNCLMSANLLQGKDAFPSGTQKYLISSVMRHATNMCRMYNDFGSIARDNAERNVNSIHFPEFTLCNGTSQNLDERKERLLKIATYEQGYLDRALEALERQSRDDAGDRAGSKDMRKLKIVKLFCDVTDLYDQLYVIKDLSSSMK

SEQ ID NO: 59 - DNA, 1542 bp, Artificial Sequence
  Codon-optimized KO
atggatgctgtgacgggtttgttaactgtcccagcaaccgctataactattggtggaactgctgtagcattggcggtagcgctaatcttttggtacctgaaatcctacacatcagctagaagatcccaatcaaatcatcttccaagagtgcctgaagtcccaggtgttccattgttaggaaatctgttacaattgaaggagaaaaagccatacatgacttttacgagatgggcagcgacatatggacctatctatagtatcaaaactggggctacaagtatggttgtggtatcatctaatgagatagccaaggaggcattggtgaccagattccaatccatatctacaaggaacttatctaaagccctgaaagtacttacagcagataagacaatggtcgcaatgtcagattatgatgattatcataaaacagttaagagacacatactgaccgccgtcttgggtcctaatgcacagaaaaagcatagaattcacagagatatcatgatggataacatatctactcaacttcatgaattcgtgaaaaacaacccagaacaggaagaggtagaccttagaaaaatctttcaatctgagttattcggcttagctatgagacaagccttaggaaaggatgttgaaagtttgtacgttgaagacctgaaaatcactatgaatagagacgaaatctttcaagtccttgttgttgatccaatgatgggagcaatcgatgttgattggagagacttctttccatacctaaagtgggtcccaaacaaaaagttcgaaaatactattcaacaaatgtacatcagaagagaagctgttatgaaatctttaatcaaagagcacaaaaagagaatagcgtcaggcgaaaagctaaatagttatatcgattaccttttatctgaagctcaaactttaaccgatcagcaactattgatgtccttgtgggaaccaatcattgaatcttcagatacaacaatggtcacaacagaatgggcaatgtacgaattagctaaaaaccctaaattgcaagataggttgtacagagacattaagtccgtctgtggatctgaaaagataaccgaagagcatctatcacagctgccttacattacagctattttccacgaaacactgagaagacactcaccagttcctatcattcctctaagacatgtacatgaagataccgttctaggcggctaccatgttcctgctggcacagaacttgccgttaacatctacggttgcaacatggacaaaaacgtttgggaaaatccagaggaatggaacccagaaagattcatgaaagagaatgagacaattgattttcaaaagacgatggccttcggtggtggtaagagagtttgtgctggttccttgcaagcccttttaactgcatctattgggattgggagaatggttcaagagttcgaatggaaactgaaggatatgactcaagaggaagtgaacacgataggcctaactacacaaatgttaagaccattgagagctattatcaaacctaggatctaa

SEQ ID NO: 60 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 61 - DNA, 1566 bp, Artificial Sequence
  Codon-optimized KO
aagcttactagtaaaatggacggtgtcatcgatatgcaaaccattccattgagaaccgctattgctattggtggtactgctgttgctttggttgttgcattatacttttggttcttgagatcctacgcttccccatctcatcattctaatcatttgccaccagtacctgaagttccaggtgttccagttttgggtaatttgttgcaattgaaagaaaaaaagccttacatgaccttcaccaagtgggctgaaatgtatggtccaatctactctattagaactggtgctacttccatggttgttgtctcttctaacgaaatcgccaaagaagttgttgttaccagattcccatctatctctaccagaaaattgtcttacgccttgaaggttttgaccgaagataagtctatggttgccatgtctgattatcacgattaccataagaccgtcaagagacatattttgactgctgttttgggtccaaacgcccaaaaaaagtttagagcacatagagacaccatgatggaaaacgtttccaatgaattgcatgccttcttcgaaaagaacccaaatcaagaagtcaacttgagaaagatcttccaatcccaattattcggtttggctatgaagcaagccttgggtaaagatgttgaatccatctacgttaaggatttggaaaccaccatgaagagagaagaaatcttcgaagttttggttgtcgatccaatgatgggtgctattgaagttgattggagagactttttcccatacttgaaatgggttccaaacaagtccttcgaaaacatcatccatagaatgtacactagaagagaagctgttatgaaggccttgatccaagaacacaagaaaagaattgcctccggtgaaaacttgaactcctacattgattacttgttgtctgaagcccaaaccttgaccgataagcaattattgatgtctttgtgggaacctattatcgaatcttctgataccactatggttactactgaatgggctatgtacgaattggctaagaatccaaacatgcaagacagattatacgaagaaatccaatccgtttgcggttccgaaaagattactgaagaaaacttgtcccaattgccatacttgtacgctgttttccaagaaactttgagaaagcactgtccagttcctattatgccattgagatatgttcacgaaaacaccgttttgggtggttatcatgttccagctggtactgaagttgctattaacatctacggttgcaacatggataagaaggtctgggaaaatccagaagaatggaatccagaaagattcttgtccgaaaaagaatccatggacttgtacaaaactatggcttttggtggtggtaaaagagtttgcgctggttctttacaagccatggttatttcttgcattggtatcggtagattggtccaagattttgaatggaagttgaaggatgatgccgaagaagatgttaacactttgggtttgactacccaaaagttgcatccattattggccttgattaacccaagaaagtaactcgagccgcgg

SEQ ID NO: 62 - protein, 512 aa, Lactuca sativa
MDGVIDMQTIPLRTAIAIGGTAVALVVALYFWFLRSYASPSHHSNHLPPVPEVPGVPVLGNLLQLKEKKPYMTFTKWAEMYGPIYSIRTGATSMVVVSSNEIAKEVVVTRFPSISTRKLSYALKVLTEDKSMVAMSDYHDYHKTVKRHILTAVLGPNAQKKFRAHRDTMMENVSNELHAFFEKNPNQEVNLRKIFQSQLFGLAMKQALGKDVESIYVKDLETTMKREEIFEVLVVDPMMGAIEVDWRDFFPYLKWVPNKSFENIIHRMYTRREAVMKALIQEHKKRIASGENLNSYIDYLLSEAQTLTDKQLLMSLWEPIIESSDTTMVTTEWAMYELAKNPNMQDRLYEEIQSVCGSEKITEENLSQLPYLYAVFQETLRKHCPVPIMPLRYVHENTVLGGYHVPAGTEVAINIYGCNMDKKVWENPEEWNPERFLSEKESMDLYKTMAFGGGKRVCAGSLQAMVISCIGIGRLVQDFEWKLKDDAEEDVNTLGLTTQKLHPLLALINPRK

SEQ ID NO: 63 - DNA, 1535 bp, Rubus suavissimus
atggccaccctccttgagcatttccaagctatgccctttgccatccctattgcactggctgctctgtcttggctgttcctcttttacatcaaagtttcattcttttccaacaagagtgctcaggctaagctccctcctgtgccagtggttcctgggctgccggtgattgggaatttactgcaactcaaggagaagaaaccctaccagacttttacaaggtgggctgaggagtatggaccaatctattctatcaggactggtgcttccaccatggtcgttctcaataccacccaagttgcaaaagaggccatggtgaccagatatttatccatctcaaccagaaagctatcaaacgcactaaagattcttactgctgataaatgtatggttgcaataagtgactacaacgattttcacaagatgataaagcgatacatactctcaaatgttcttggacctagtgctcagaagcgtcaccggagcaacagagataccttgagagctaatgtctgcagccgattgcattctcaagtaaagaactctcctcgagaagctgtgaatttcagaagagtttttgagtgggaactctttggaattgcattgaagcaagcctttggaaaggacatagaaaagcccatttatgtggaggaacttggcactacactgtcaagagatgagatctttaaggttctagtgcttgacataatggagggtgcaattgaggttgattggagagatttcttcccttacctgagatggattccgaatacgcgcatggaaacaaaaattcagcgactctatttccgcaggaaagcagtgatgactgccctgatcaacgagcagaagaagcgaattgcttcaggagaggaaatcaactgttatatcgacttcttgcttaaggaagggaagacactgacaatggaccaaataagtatgttgctttgggagacggttattgaaacagcagatactacaatggtaacgacagaatgggctatgtatgaagttgctaaagactcaaagcgtcaggatcgtctctatcaggaaatccaaaaggtttgtggatcggagatggttacagaggaatacttgtcccaactgccgtacctgaatgcagttttccatgaaacgctaaggaagcacagtccggctgcgttagttcctttaagatatgcacatgaagatacccaactaggaggttactacattccagctggaactgagattgctataaacatatacgggtgtaacatggacaagcatcaatgggaaagccctgaggaatggaaaccggagagatttttggacccgaaatttgatcctatggatttgtacaagaccatggcttttggggctggaaagagggtatgtgctggttctcttcaggcaatgttaatagcgtgcccgacgattggtaggctggtgcaggagtttgagtggaagctgagagatggagaagaagaaaatgtagatactgttgggctcaccactcacaaacgctatccaatgcatgcaatcctgaagccaagaagtta

SEQ ID NO: 64 - DNA, 1536 bp, Artificial Sequence
  Codon-optimized KO
atggctaccttgttggaacattttcaagctatgccattcgctattccaattgctttggctgctttgtcttggttgtttttgttctacatcaaggtttctttcttctccaacaaatccgctcaagctaaattgccaccagttccagttgttccaggtttgccagttattggtaatttgttgcaattgaaagaaaagaagccataccaaaccttcactagatgggctgaagaatatggtccaatctactctattagaactggtgcttctactatggttgtcttgaacactactcaagttgccaaagaagctatggttaccagatacttgtctatctctaccagaaagttgtccaacgccttgaaaattttgaccgctgataagtgcatggttgccatttctgattacaacgatttccacaagatgatcaagagatatatcttgtctaacgttttgggtccatctgcccaaaaaagacatagatctaacagagataccttgagagccaacgtttgttctagattgcattcccaagttaagaactctccaagagaagctgtcaactttagaagagttttcgaatgggaattattcggtatcgctttgaaacaagccttcggtaaggatattgaaaagccaatctacgtcgaagaattgggtactactttgtccagagatgaaatcttcaaggttttggtcttggacattatggaaggtgccattgaagttgattggagagattttttcccatacttgcgttggattccaaacaccagaatggaaactaagatccaaagattatactttagaagaaaggccgttatgaccgccttgattaacgaacaaaagaaaagaattgcctccggtgaagaaatcaactgctacatcgatttcttgttgaaagaaggtaagaccttgaccatggaccaaatctctatgttgttgtgggaaaccgttattgaaactgctgataccacaatggttactactgaatgggctatgtacgaagttgctaaggattctaaaagacaagacagattataccaagaaatccaaaaggtctgcggttctgaaatggttacagaagaatacttgtcccaattgccatacttgaatgctgttttccacgaaactttgagaaaacattctccagctgctttggttccattgagatatgctcatgaagatactcaattgggtggttattacattccagccggtactgaaattgccattaacatctacggttgcaacatggacaaacaccaatgggaatctccagaagaatggaagccagaaagatttttggatcctaagtttgacccaatggacttgtacaaaactatggcttttggtgctggtaaaagagtttgcgctggttctttacaagctatgttgattgcttgtccaaccatcggtagattggttcaagaatttgaatggaagttgagagatggtgaagaagaaaacgttgatactgttggtttgaccacccataagagatatccaatgcatgctattttgaagccaagatcttaa

SEQ ID NO: 65 - DNA, 1572 bp, Artificial Sequence
  Codon-optimized KO
aagcttactagtaaaatggcctccatcacccatttcttacaagattttcaagctactccattcgctactgcttttgctgttggtggtgtttctttgttgatattcttcttcttcatccgtggtttccactctactaagaaaaacgaatattacaagttgccaccagttccagttgttccaggtttgccagttgttggtaatttgttgcaattgaaagaaaagaagccatacaagactttcttgagatgggctgaaattcatggtccaatctactctattagaactggtgcttctaccatggttgttgttaactctactcatgttgccaaagaagctatggttaccagattctcttcaatctctaccagaaagttgtccaaggctttggaattattgacctccaacaaatctatggttgccacctctgattacaacgaatttcacaagatggtcaagaagtacatcttggccgaattattgggtgctaatgctcaaaagagacacagaattcatagagacaccttgatcgaaaacgtcttgaacaaattgcatgcccataccaagaattctccattgcaagctgttaacttcagaaagatcttcgaatctgaattattcggtttggctatgaagcaagccttgggttatgatgttgattccttgttcgttgaagaattgggtactaccttgtccagagaagaaatctacaacgttttggtcagtgacatgttgaagggtgctattgaagttgattggagagactttttcccatacttgaaatggatcccaaacaagtccttcgaaatgaagattcaaagattggcctctagaagacaagccgttatgaactctattgtcaaagaacaaaagaagtccattgcctctggtaagggtgaaaactgttacttgaattacttgttgtccgaagctaagactttgaccgaaaagcaaatttccattttggcctgggaaaccattattgaaactgctgatacaactgttgttaccactgaatgggctatgtacgaattggctaaaaacccaaagcaacaagacagattatacaacgaaatccaaaacgtctgcggtactgataagattaccgaagaacatttgtccaagttgccttacttgtctgctgtttttcacgaaaccttgagaaagtattctccatctccattggttccattgagatacgctcatgaagatactcaattgggtggttattatgttccagccggtactgaaattgctgttaatatctacggttgcaacatggacaagaatcaatgggaaactccagaagaatggaagccagaaagatttttggacgaaaagtacgatccaatggacatgtacaagactatgtcttttggttccggtaaaagagtttgcgctggttctttacaagctagtttgattgcttgtacctccatcggtagattggttcaagaatttgaatggagattgaaagacggtgaagttgaaaacgttgataccttgggtttgactacccataagttgtatccaatgcaagctatcttgcaacctagaaactgactcgagccgcgg

SEQ ID NO: 66 - protein, 514 aa, Castanea mollissima
MASITHFLQDFQATPFATAFAVGGVSLLIFFFFIRGFHSTKKNEYYKLPPVPVVPGLPVVGNLLQLKEKKPYKTFLRWAEIHGPIYSIRTGASTMVVVNSTHVAKEAMVTRFSSISTRKLSKALELLTSNKSMVATSDYNEFHKMVKKYILAELLGANAQKRHRIHRDTLIENVLNKLHAHTKNSPLQAVNFRKIFESELFGLAMKQALGYDVDSLFVEELGTTLSREEIYNVLVSDMLKGAIEVDWRDFFPYLKWIPNKSFEMKIQRLASRRQAVMNSIVKEQKKSIASGKGENCYLNYLLSEAKTLTEKQISILAWETIIETADTTVVTTEWAMYELAKNPKQQDRLYNEIQNVCGTDKITEEHLSKLPYLSAVFHETLRKYSPSPLVPLRYAHEDTQLGGYYVPAGTEIAVNIYGCNMDKNQWETPEEWKPERFLDEKYDPMDMYKTMSFGSGKRVCAGSLQASLIACTSIGRLVQEFEWRLKDGEVENVDTLGLTTHKLYPMQAILQPRN

SEQ ID NO: 67 - DNA, 1512 bp, Artificial Sequence
  Codon-optimized KO
atgatttccttgttgttgggttttgttgtctcctccttcttgtttatcttcttcttgaaaaaattgttgttcttcttcagtcgtcacaaaatgtccgaagtttctagattgccatctgttccagttccaggttttccattgattggtaacttgttgcaattgaaagaaaagaagccacacaagactttcaccaagtggtctgaattatatggtccaatctactctatcaagatgggttcctcttctttgatcgtcttgaactctattgaaaccgccaaagaagctatggtcagtagattctcttcaatctctaccagaaagttgtctaacgctttgactgttttgacctgcaacaaatctatggttgctacctctgattacgatgactttcataagttcgtcaagagatgcttgttgaacggtttgttgggtgctaatgctcaagaaagaaaaagacattacagagatgccttgatcgaaaacgttacctctaaattgcatgcccataccagaaatcatccacaagaaccagttaacttcagagccattttcgaacacgaattattcggtgttgctttgaaacaagccttcggtaaagatgtcgaatccatctatgtaaaagaattgggtgtcaccttgtccagagatgaaattttcaaggttttggtccacgacatgatggaaggtgctattgatgttgattggagagatttcttcccatacttgaaatggatcccaaacaactctttcgaagccagaattcaacaaaagcacaagagaagattggctgttatgaacgccttgatccaagacagattgaatcaaaacgattccgaatccgatgatgactgctacttgaatttcttgatgtctgaagctaagaccttgaccatggaacaaattgctattttggtttgggaaaccattatcgaaactgctgataccactttggttactactgaatgggctatgtacgaattggccaaacatcaatctgttcaagatagattattcaaagaaatccaatccgtctgcggtggtgaaaagatcaaagaagaacaattgccaagattgccttacgtcaatggtgtttttcacgaaaccttgagaaagtattctccagctccattggttccaattagatacgctcatgaagatacccaaattggtggttatcatattccagccggttctgaaattgccattaacatctacggttgcaacatggataagaagagatgggaaagacctgaagaatggtggccagaaagatttttggaagatagatacgaatcctccgacttgcataagactatggcttttggtgctggtaaaagagtttgtgctggtgctttacaagctagtttgatggctggtattgctatcggtagattggttcaagaattcgaatggaagttgagagatggtgaagaagaaaacgttgatacttacggtttgacctcccaaaagttgtatccattgatggccattatcaacccaagaagatcttaa

SEQ ID NO: 68 - protein, 506 aa, Thellungiella halophila
MASMISLLLGFVVSSFLFIFFLKKLLFFFSRHKMSEVSRLPSVPVPGFPLIGNLLQLKEKKPHKTFTKWSELYGPIYSIKMGSSSLIVLNSIETAKEAMVSRFSSISTRKLSNALTVLTCNKSMVATSDYDDFHKFVKRCLLNGLLGANAQERKRHYRDALIENVTSKLHAHTRNHPQEPVNFRAIFEHELFGVALKQAFGKDVESIYVKELGVTLSRDEIFKVLVHDMMEGAIDVDWRDFFPYLKWIPNNSFEARIQQKHKRRLAVMNALIQDRLNQNDSESDDDCYLNFLMSEAKTLTMEQIAILVWETIIETADTTLVTTEWAMYELAKHQSVQDRLFKEIQSVCGGEKIKEEQLPRLPYVNGVFHETLRKYSPAPLVPIRYAHEDTQIGGYHIPAGSEIAINIYGCNMDKKRWERPEEWWPERFLEDRYESSDLHKTMAFGAGKRVCAGALQASLMAGIAIGRLVQEFEWKLRDGEEENVDTYGLTSQKLYPLMAIINPRRS

SEQ ID NO: 69 - DNA, 1554 bp, Artificial Sequence
  Codon-optimized KO
aagcttactagtaaaatggacatgatgggtattgaagctgttccatttgctactgctgttgttttgggtggtatttccttggttgttttgatcttcatcagaagattcgtttccaacagaaagagatccgttgaaggtttgccaccagttccagatattccaggtttaccattgattggtaacttgttgcaattgaaagaaaagaagccacataagacctttgctagatgggctgaaacttacggtccaattttctctattagaactggtgcttctaccatgatcgtcttgaattcttctgaagttgccaaagaagctatggtcactagattctcttcaatctctaccagaaagttgtccaacgccttgaagattttgaccttcgataagtgtatggttgccacctctgattacaacgattttcacaaaatggtcaagggtttcatcttgagaaacgttttaggtgctccagcccaaaaaagacatagatgtcatagagataccttgatcgaaaacatctctaagtacttgcatgcccatgttaagacttctccattggaaccagttgtcttgaagaagattttcgaatccgaaattttcggtttggctttgaaacaagccttgggtaaggatatcgaatccatctatgttgaagaattgggtactaccttgtccagagaagaaatttttgccgttttggttgttgatccaatggctggtgctattgaagttgattggagagattttttcccatacttgtcctggattccaaacaagtctatggaaatgaagatccaaagaatggattttagaagaggtgctttgatgaaggccttgattggtgaacaaaagaaaagaatcggttccggtgaagaaaagaactcctacattgatttcttgttgtctgaagctaccactttgaccgaaaagcaaattgctatgttgatctgggaaaccatcatcgaaatttccgatacaactttggttacctctgaatgggctatgtacgaattggctaaagacccaaatagacaagaaatcttgtacagagaaatccacaaggtttgcggttctaacaagttgactgaagaaaacttgtccaagttgccatacttgaactctgttttccacgaaaccttgagaaagtattctccagctccaatggttccagttagatatgctcatgaagatactcaattgggtggttaccatattccagctggttctcaaattgccattaacatctacggttgcaacatgaacaaaaagcaatgggaaaatcctgaagaatggaagccagaaagattcttggacgaaaagtatgacttgatggacttgcataagactatggcttttggtggtggtaaaagagtttgtgctggtgctttacaagcaatgttgattgcttgcacttccatcggtagattcgttcaagaatttgaatggaagttgatgggtggtgaagaagaaaacgttgatactgttgctttgacctcccaaaaattgcatccaatgcaagccattattaaggccagagaatgactcgagccgcgg

SEQ ID NO: 70 - protein, 508 aa, Vitis vinifera
MDMMGIEAVPFATAVVLGGISLVVLIFIRRFVSNRKRSVEGLPPVPDIPGLPLIGNLLQLKEKKPHKTFARWAETYGPIFSIRTGASTMIVLNSSEVAKEAMVTRFSSISTRKLSNALKILTFDKCMVATSDYNDFHKMVKGFILRNVLGAPAQKRHRCHRDTLIENISKYLHAHVKTSPLEPVVLKKIFESEIFGLALKQALGKDIESIYVEELGTTLSREEIFAVLVVDPMAGAIEVDWRDFFPYLSWIPNKSMEMKIQRMDFRRGALMKALIGEQKKRIGSGEEKNSYIDFLLSEATTLTEKQIAMLIWETIIEISDTTLVTSEWAMYELAKDPNRQEILYREIHKVCGSNKLTEENLSKLPYLNSVFHETLRKYSPAPMVPVRYAHEDTQLGGYHIPAGSQIAINIYGCNMNKKQWENPEEWKPERFLDEKYDLMDLHKTMAFGGGKRVCAGALQAMLIACTSIGRFVQEFEWKLMGGEEENVDTVALTSQKLHPMQAIIKARE

SEQ ID NO: 71 - DNA, 1593 bp, Artificial Sequence
  Codon-optimized KO
aagcttaaaatgagtaagtctaatagtatgaattctacatcacacgaaaccctttttcaacaattggtcttgggtttggaccgtatgccattgatggatgttcactggttgatctacgttgctttcggcgcatggttatgttcttatgtgatacatgttttatcatcttcctctacagtaaaagtgccagttgttggatacaggtctgtattcgaacctacatggttgcttagacttagattcgtctgggaaggtggctctatcataggtcaagggtacaataagtttaaagactctattttccaagttaggaaattgggaactgatattgtcattataccacctaactatattgatgaagtgagaaaattgtcacaggacaagactagatcagttgaacctttcattaatgattttgcaggtcaatacacaagaggcatggttttcttgcaatctgacttacaaaaccgtgttatacaacaaagactaactccaaaattggtttccttgaccaaggtcatgaaggaagagttggattatgctttaacaaaagagatgcctgatatgaaaaatgacgaatgggtagaagtagatatcagtagtataatggtgagattgatttccaggatctccgccagagtctttctagggcctgaacactgtcgtaaccaggaatggttgactactacagcagaatattcagaatcacttttcattacagggtttatcttaagagttgtacctcatatcttaagaccattcatcgcccctctattaccttcatacaggactctacttagaaacgtttcaagtggtagaagagtcatcggtgacatcataagatctcagcaaggggatggtaacgaagatatactttcctggatgagagatgctgccacaggagaggaaaagcaaatcgataacattgctcagagaatgttaattctttctttagcatcaatccacactactgcgatgaccatgacacatgccatgtacgatctatgtgcttgccctgagtacattgaaccattaagagatgaagttaaatctgttgttggggcttctggctgggacaagacagcgttaaacagatttcataagttggactccttcctaaaagagtcacaaagattcaacccagtattcttattgacattcaatagaatctaccatcaatctatgaccttatcagatggcactaacattccatctggaacacgtattgctgttccatcacacgcaatgttgcaagattctgcacatgtcccaggtccaaccccacctactgaatttgatggattcagatatagtaagatacgttctgatagtaactacgcacaaaagtacctattctccatgaccgattcttcaaacatggctttcggatacggcaagtatgcttgtccaggtagattttacgcgtctaatgagatgaaactaacattagccattttgttgctacaatttgagttcaaactaccagatggtaaaggtcgtcctagaaatatcactatcgattctgatatgattccagacccaagagctagactttgcgtcagaaaaagatcacttagagatgaatgaccgcgg

SEQ ID NO: 72 - protein, 525 aa, Gibberella fujikuroi
MSKSNSMNSTSHETLFQQLVLGLDRMPLMDVHWLIYVAFGAWLCSYVIHVLSSSSTVKVPVVGYRSVFEPTWLLRLRFVWEGGSIIGQGYNKFKDSIFQVRKLGTDIVIIPPNYIDEVRKLSQDKTRSVEPFINDFAGQYTRGMVFLQSDLQNRVIQQRLTPKLVSLTKVMKEELDYALTKEMPDMKNDEWVEVDISSIMVRLISRISARVFLGPEHCRNQEWLTTTAEYSESLFITGFILRVVPHILRPFIAPLLPSYRTLLRNVSSGRRVIGDIIRSQQGDGNEDILSWMRDAATGEEKQIDNIAQRMLILSLASIHTTAMTMTHAMYDLCACPEYIEPLRDEVKSVVGASGWDKTALNRFHKLDSFLKESQRFNPVFLLTFNRIYHQSMTLSDGTNIPSGTRIAVPSHAMLQDSAHVPGPTPPTEFDGFRYSKIRSDSNYAQKYLFSMTDSSNMAFGYGKYACPGRFYASNEMKLTLAILLLQFEFKLPDGKGRPRNITIDSDMIPDPRARLCVRKRSLRDE

SEQ ID NO: 73 - DNA, 1515 bp, Artificial Sequence
  Codon-optimized KO
aagcttaaaatggaagatcctactgtcttatatgcttgtcttgccattgcagttgcaactttcgttgttagatggtacagagatccattgagatccatcccaacagttggtggttccgatttgcctattctatcttacatcggcgcactaagatggacaagacgtggcagagagatacttcaagagggatatgatggctacagaggatctacattcaaaatcgcgatgttagaccgttggatcgtgatcgcaaatggtcctaaactagctgatgaagtcagacgtagaccagatgaagagttaaactttatggacggattaggagcattcgtccaaactaagtacaccttaggtgaagctattcataacgatccataccatgtcgatatcataagagaaaaactaacaagaggccttccagccgtgcttcctgatgtcattgaagagttgacacttgcggttagacagtacattccaacagaaggtgatgaatgggtgtccgtaaactgttcaaaggccgcaagagatattgttgctagagcttctaatagagtctttgtaggtttgcctgcttgcagaaaccaaggttacttagatttggcaatagactttacattgtctgttgtcaaggatagagccatcatcaatatgtttccagaattgttgaagccaatagttggcagagttgtaggtaacgccaccagaaatgttcgtagagctgttccttttgttgctccattggtggaggaaagacgtagacttatggaagagtacggtgaagactggtctgaaaaacctaatgatatgttacagtggataatggatgaagctgcatccagagatagttcagtgaaggcaatcgcagagagattgttaatggtgaacttcgcggctattcatacctcatcaaacactatcactcatgctttgtaccaccttgccgaaatgcctgaaactttgcaaccacttagagaagagatcgaaccattagtcaaagaggagggctggaccaaggctgctatgggaaaaatgtggtggttagattcatttctaagagaatctcaaagatacaatggcattaacatcgtatctttaactagaatggctgacaaagatattacattgagtgatggcacatttttgccaaaaggtactctagtggccgttccagcgtattctactcatagagatgatgctgtctacgctgatgccttagtattcgatcctttcagattctcacgtatgagagcgagagaaggtgaaggtacaaagcaccagttcgttaatacttcagtcgagtacgttccatttggtcacggaaagcatgcttgtccaggaagattcttcgccgcaaacgaattgaaagcaatgttggcttacattgttctaaactatgatgtaaagttgcctggtgacggtaaacgtccattgaacatgtattggggtccaacagttttgcctgcaccagcaggccaagtattgttcagaaagagacaagttagtctataaccgcgg

SEQ ID NO: 74 - protein, 499 aa, Trametes versicolor
MEDPTVLYACLAIAVATFVVRWYRDPLRSIPTVGGSDLPILSYIGALRWTRRGREILQEGYDGYRGSTFKIAMLDRWIVIANGPKLADEVRRRPDEELNFMDGLGAFVQTKYTLGEAIHNDPYHVDIIREKLTRGLPAVLPDVIEELTLAVRQYIPTEGDEWVSVNCSKAARDIVARASNRVFVGLPACRNQGYLDLAIDFTLSVVKDRAIINMFPELLKPIVGRVVGNATRNVRRAVPFVAPLVEERRRLMEEYGEDWSEKPNDMLQWIMDEAASRDSSVKAIAERLLMVNFAAIHTSSNTITHALYHLAEMPETLQPLREEIEPLVKEEGWTKAAMGKMWWLDSFLRESQRYNGINIVSLTRMADKDITLSDGTFLPKGTLVAVPAYSTHRDDAVYADALVFDPFRFSRMRAREGEGTKHQFVNTSVEYVPFGHGKHACPGRFFAANELKAMLAYIVLNYDVKLPGDGKRPLNMYWGPTVLPAPAGQVLFRKRQVSL

SEQ ID NO: 75 - DNA, 1530 bp, Artificial Sequence
  Codon-optimized KO
atggcatttttctctatgatttcaattttgttgggatttgttatttcttctttcatcttcatctttttcttcaaaaagttacttagttttagtaggaaaaacatgtcagaagtttctactttgccaagtgttccagtagtgcctggttttccagttattgggaatttgttgcaactaaaggagaaaaagcctcataaaactttcactagatggtcagagatatatggacctatctactctataaagatgggttcttcatctcttattgtattgaacagtacagaaactgctaaggaagcaatggtcactagattttcatcaatatctaccagaaaattgtcaaacgccctaacagttctaacctgcgataagtctatggtcgccacttctgattatgatgacttccacaaattagttaagagatgtttgctaaatggacttcttggtgctaatgctcaaaagagaaaaagacactacagagatgctttgattgaaaatgtgagttccaagctacatgcacacgctagagatcatccacaagagccagttaactttagagcaattttcgaacacgaattgtttggtgtagcattaaagcaagccttcggtaaagacgtagaatccatatacgtcaaggagttaggcgtaacattatcaaaagatgaaatctttaaggtgcttgtacatgatatgatggagggtgcaattgatgtagattggagagatttcttcccatatttgaaatggatccctaataagtcttttgaagctaggatacaacaaaagcacaagagaagactagctgttatgaacgcacttatacaggacagattgaagcaaaatgggtctgaatcagatgatgattgttaccttaacttcttaatgtctgaggctaaaacattgactaaggaacagatcgcaatccttgtctgggaaacaatcattgaaacagcagatactaccttagtcacaactgaatgggccatatacgagctagccaaacatccatctgtgcaagataggttgtgtaaggagatccagaacgtgtgtggtggagagaaattcaaggaagagcagttgtcacaagttccttaccttaacggcgttttccatgaaaccttgagaaaatactcacctgcaccattagttcctattagatacgcccacgaagatacacaaatcggtggctaccatgttccagctgggtccgaaattgctataaacatctacgggtgcaacatggacaaaaagagatgggaaagaccagaagattggtggccagaaagattcttagatgatggcaaatatgaaacatctgatttgcataaaacaatggctttcggagctggcaaaagagtgtgtgccggtgctctacaagcctccctaatggctggtatcgctattggtagattggtccaagagttcgaatggaaacttagagatggtgaagaggaaaatgtcgatacttatgggttaacatctcaaaagttatacccactaatggcaatcatcaatcctagaagatcctaa

SEQ ID NO: 76 - protein, 509 aa, Arabidopsis thaliana
MAFFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 77 - DNA, 2133 bp, Artificial Sequence
  Codon-optimized CPR
atgcaatcagattcagtcaaagtctctccatttgatttggtttccgctgctatgaatggcaaggcaatggaaaagttgaacgctagtgaatctgaagatccaacaacattgcctgcactaaagatgctagttgaaaatagagaattgttgacactgttcacaacttccttcgcagttcttattgggtgtcttgtatttctaatgtggagacgttcatcctctaaaaagctggtacaagatccagttccacaagttatcgttgtaaagaagaaagagaaggagtcagaggttgatgacgggaaaaagaaagtttctattttctacggcacacaaacaggaactgccgaaggttttgctaaagcattagtcgaggaagcaaaagtgagatatgaaaagacctctttcaaggttatcgatctagatgactacgctgcagatgatgatgaatatgaggaaaaactgaaaaaggaatccttagccttcttcttcttggccacatacggtgatggtgaacctactgataatgctgctaacttctacaagtggttcacagaaggcgacgataaaggtgaatggctgaaaaagttacaatacggagtatttggtttaggtaacagacaatatgaacatttcaacaagatcgctattgtagttgatgataaacttactgaaatgggagccaaaagattagtaccagtaggattaggggatgatgatcagtgtatagaagatgacttcaccgcctggaaggaattggtatggccagaattggatcaacttttaagggacgaagatgatacttctgtgactaccccatacactgcagccgtattggagtacagagtggtttaccatgataaaccagcagactcatatgctgaagatcaaacccatacaaacggtcatgttgttcatgatgcacagcatccttcaagatctaatgtggctttcaaaaaggaactacacacctctcaatcagataggtcttgtactcacttagaattcgatatttctcacacaggactgtcttacgaaactggcgatcacgttggcgtttattccgagaacttgtccgaagttgtcgatgaagcactaaaactgttagggttatcaccagacacatacttctcagtccatgctgataaggaggatgggacacctatcggtggtgcttcactaccaccaccttttcctccttgcacattgagagacgctctaaccagatacgcagatgtcttatcctcacctaaaaaggtagctttgctggcattggctgctcatgctagtgatcctagtgaagccgataggttaaagttcctggcttcaccagccggaaaagatgaatatgcacaatggatcgtcgccaaccaacgttctttgctagaagtgatgcaaagttttccatctgccaagcctccattaggtgtgttcttcgcagcagtagctccacgtttacaaccaagatactactctatcagttcatctcctaagatgtctcctaacagaatacatgttacatgtgctttggtgtacgagactactccagcaggcagaattcacagaggattgtgttcaacctggatgaaaaatgctgtccctttaacagagtcacctgattgctctcaagcatccattttcgttagaacatcaaatttcagacttccagtggatccaaaagttccagtcattatgataggaccaggcactggtcttgccccattcaggggctttcttcaagagagattggccttgaaggaatctggtacagaattgggttcttctatctttttctttggttgccgtaatagaaaagttgactttatctacgaggacgagcttaacaattttgttgagacaggagcattgtcagaattgatcgtcgcattttcaagagaagggactgccaaagagtacgttcagcacaagatgagtcaaaaagcctccgatatatggaaacttctaagtgaaggtgcctatctttatgtctgtggcgatgcaaagggcatggccaaggatgtccatagaactctgcatacaattgttcaggaacaagggagtctggattcttccaaggctgaattgtacgtcaaaaacttacagatgtctggaagatacttaagagatgtttggtaa

SEQ ID NO: 78 - protein, 710 aa, Stevia rebaudiana
MQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 79 - DNA, 2106 bp, Siraitia grosvenorii
atgaaggtcagtccattcgaattcatgtccgctattatcaagggtagaatggacccatctaactcctcatttgaatctactggtgaagttgcctccgttatctttgaaaacagagaattggttgccatcttgaccacttctattgctgttatgattggttgcttcgttgtcttgatgtggagaagagctggttctagaaaggttaagaatgtcgaattgccaaagccattgattgtccatgaaccagaacctgaagttgaagatggtaagaagaaggtttccatcttcttcggtactcaaactggtactgctgaaggttttgctaaggctttggctgatgaagctaaagctagatacgaaaaggctaccttcagagttgttgatttggatgattatgctgccgatgatgaccaatacgaagaaaaattgaagaacgaatccttcgccgttttcttgttggctacttatggtgatggtgaacctactgataatgctgctagattttacaagtggttcgccgaaggtaaagaaagaggtgaatggttgcaaaacttgcactatgctgtttttggtttgggtaacagacaatacgaacacttcaacaagattgctaaggttgccgacgaattattggaagctcaaggtggtaatagattggttaaggttggtttaggtgatgacgatcaatgcatcgaagatgatttttctgcttggagagaatctttgtggccagaattggatatgttgttgagagatgaagatgatgctactactgttactactccatatactgctgctgtcttggaatacagagttgtctttcatgattctgctgatgttgctgctgaagataagtcttggattaacgctaatggtcatgctgttcatgatgctcaacatccattcagatctaacgttgtcgtcagaaaagaattgcatacttctgcctctgatagatcctgttctcatttggaattcaacatttccggttccgctttgaattacgaaactggtgatcatgttggtgtctactgtgaaaacttgactgaaactgttgatgaagccttgaacttgttgggtttgtctccagaaacttacttctctatctacaccgataacgaagatggtactccattgggtggttcttcattgccaccaccatttccatcatgtactttgagaactgctttgaccagatacgctgatttgttgaactctccaaaaaagtctgctttgttggctttagctgctcatgcttctaatccagttgaagctgatagattgagatacttggcttctccagctggtaaagatgaatatgcccaatctgttatcggttcccaaaagtctttgttggaagttatggctgaattcccatctgctaaaccaccattaggtgttttttttgctgctgttgctccaagattgcaacctagattctactccatttcatcctctccaagaatggctccatctagaatccatgttacttgtgctttggtttacgataagatgccaactggtagaattcataagggtgtttgttctacctggatgaagaattctgttccaatggaaaagtcccatgaatgttcttgggctccaattttcgttagacaatccaattttaagttgccagccgaatccaaggttccaattatcatggttggtccaggtactggtttggctccttttagaggttttttacaagaaagattggccttgaaagaatccggtgttgaattgggtccatccattttgtttttcggttgcagaaacagaagaatggattacatctacgaagatgaattgaacaacttcgttgaaaccggtgctttgtccgaattggttattgctttttctagagaaggtcctaccaaagaatacgtccaacataagatggctgaaaaggcttctgatatctggaacttgatttctgaaggtgcttacttgtacgtttgtggtgatgctaaaggtatggctaaggatgttcatagaaccttgcataccatcatgcaagaacaaggttctttggattcttccaaagctgaatccatggtcaagaacttgcaaatgaatggtagatacttaagagatgtttggtaa

SEQ ID NO: 80 - protein, 701 aa, Siraitia grosvenorii
MKVSPFEFMSAIIKGRMDPSNSSFESTGEVASVIFENRELVAILTTSIAVMIGCFVVLMWRRAGSRKVKNVELPKPLIVHEPEPEVEDGKKKVSIFFGTQTGTAEGFAKALADEAKARYEKATFRVVDLDDYAADDDQYEEKLKNESFAVFLLATYGDGEPTDNAARFYKWFAEGKERGEWLQNLHYAVFGLGNRQYEHFNKIAKVADELLEAQGGNRLVKVGLGDDDQCIEDDFSAWRESLWPELDMLLRDEDDATTVTTPYTAAVLEYRVVFHDSADVAAEDKSWINANGHAVHDAQHPFRSNVVVRKELHTSASDRSCSHLEFNISGSALNYETGDHVGVYCENLTETVDEALNLLGLSPETYFSIYTDNEDGTPLGGSSLPPPFPSCTLRTALTRYADLLNSPKKSALLALAAHASNPVEADRLRYLASPAGKDEYAQSVIGSQKSLLEVMAEFPSAKPPLGVFFAAVAPRLQPRFYSISSSPRMAPSRIHVTCALVYDKMPTGRIHKGVCSTWMKNSVPMEKSHECSWAPIFVRQSNFKLPAESKVPIIMVGPGTGLAPFRGFLQERLALKESGVELGPSILFFGCRNRRMDYIYEDELNNFVETGALSELVIAFSREGPTKEYVQHKMAEKASDIWNLISEGAYLYVCGDAKGMAKDVHRTLHTIMQEQGSLDSSKAESMVKNLQMNGRYLRDVW

SEQ ID NO: 81 - DNA, 2142 bp, Artificial Sequence
  Codon-optimized CPR
atggcagaattagatacacttgatatagtagtattaggtgttatctttttgggtactgtggcatactttactaagggtaaattgtggggtgttaccaaggatccatacgctaacggattcgctgcaggtggtgcttccaagcctggcagaactagaaacatcgtcgaagctatggaggaatcaggtaaaaactgtgttgttttctacggcagtcaaacaggtacagcggaggattacgcatcaagacttgcaaaggaaggaaagtccagattcggtttgaacactatgatcgccgatctagaagattatgacttcgataacttagacactgttccatctgataacatcgttatgtttgtattggctacttacggtgaaggcgaaccaacagataacgccgtggatttctatgagttcattactggcgaagatgcctctttcaatgagggcaacgatcctccactaggtaacttgaattacgttgcgttcggtctgggcaacaatacctacgaacactacaactcaatggtcaggaacgttaacaaggctctagaaaagttaggagctcatagaattggagaagcaggtgagggtgacgacggagctggaactatggaagaggactttttagcttggaaagatccaatgtgggaagccttggctaaaaagatgggcttggaggaaagagaagctgtatatgaacctattttcgctatcaatgagagagatgatttgacccctgaagcgaatgaggtatacttgggagaacctaataagctacacttggaaggtacagcgaaaggtccattcaactcccacaacccatatatcgcaccaattgcagaatcatacgaacttttctcagctaaggatagaaattgtctgcatatggaaattgatatttctggtagtaatctaaagtatgaaacaggcgaccatatcgcgatctggcctaccaacccaggtgaagaggtcaacaaatttcttgacattctagatctgtctggtaagcaacattccgtcgtaacagtgaaagccttagaacctacagccaaagttccttttccaaatccaactacctacgatgctatattgagataccatctggaaatatgcgctccagtttctagacagtttgtctcaactttagcagcattcgcccctaatgatgatatcaaagctgagatgaaccgtttgggatcagacaaagattacttccacgaaaagacaggaccacattactacaatatcgctagatttttggcctcagtctctaaaggtgaaaaatggacaaagataccattttctgctttcatagaaggccttacaaaactacaaccaagatactattctatctcttcctctagtttagttcagcctaaaaagattagtattactgctgttgtcgaatctcagcaaattccaggtagagatgacccattcagaggtgtagcgactaactacttgttcgctttgaagcagaaacaaaacggtgatccaaatccagctccttttggccaatcatacgagttgacaggaccaaggaataagtatgatggtatacatgttccagtccatgtaagacattctaactttaagctaccatctgatccaggcaaacctattatcatgatcggtccaggtaccggtgttgccccttttagaggcttcgtccaagagagggcaaaacaagccagagatggtgtagaagttggtaaaacactgctgttctttggatgtagaaagagtacagaagatttcatgtatcaaaaagagtggcaagagtacaaggaagctcttggcgacaaattcgaaatgattacagctttttcaagagaaggatctaaaaaggtttatgttcaacacagactgaaggaaagatcaaaggaagtttctgatcttctatcccaaaaagcatacttctacgtttgcggagacgccgcacatatggcacgtgaagtgaacactgtgttagcacagatcatagcagaaggccgtggtgtatcagaagccaagggtgaggaaattgtcaaaaacatgagatcagcaaatcaataccaagtgtgttctgatttcgtaactttacactgtaaagagacaacatacgcgaattcagaattgcaagaggatgtctggagttaa

SEQ ID NO: 82 - protein, 713 aa, Gibberella fujikuroi
MAELDTLDIVVLGVIFLGTVAYFTKGKLWGVTKDPYANGFAAGGASKPGRTRNIVEAMEESGKNCVVFYGSQTGTAEDYASRLAKEGKSRFGLNTMIADLEDYDFDNLDTVPSDNIVMFVLATYGEGEPTDNAVDFYEFITGEDASFNEGNDPPLGNLNYVAFGLGNNTYEHYNSMVRNVNKALEKLGAHRIGEAGEGDDGAGTMEEDFLAWKDPMWEALAKKMGLEEREAVYEPIFAINERDDLTPEANEVYLGEPNKLHLEGTAKGPFNSHNPYIAPIAESYELFSAKDRNCLHMEIDISGSNLKYETGDHIAIWPTNPGEEVNKFLDILDLSGKQHSVVTVKALEPTAKVPFPNPTTYDAILRYHLEICAPVSRQFVSTLAAFAPNDDIKAEMNRLGSDKDYFHEKTGPHYYNIARFLASVSKGEKWTKIPFSAFIEGLTKLQPRYYSISSSSLVQPKKISITAVVESQQIPGRDDPFRGVATNYLFALKQKQNGDPNPAPFGQSYELTGPRNKYDGIHVPVHVRHSNFKLPSDPGKPIIMIGPGTGVAPFRGFVQERAKQARDGVEVGKTLLFFGCRKSTEDFMYQKEWQEYKEALGDKFEMITAFSREGSKKVYVQHRLKERSKEVSDLLSQKAYFYVCGDAAHMAREVNTVLAQIIAEGRGVSEAKGEEIVKNMRSANQYQVCSDFVTLHCKETTYANSELQEDVWS

SEQ ID NO: 83 - DNA, 2130 bp, Stevia rebaudiana
atgcaatcggaatccgttgaagcatcgacgattgatttgatgactgctgttttgaaggacacagtgatcgatacagcgaacgcatctgataacggagactcaaagatgccgccggcgttggcgatgatgttcgaaattcgtgatctgttgctgattttgactacgtcagttgctgttttggtcggatgtttcgttgttttggtgtggaagagatcgtccgggaagaagtccggcaaggaattggagccgccgaagatcgttgtgccgaagaggcggctggagcaggaggttgatgatggtaagaagaaggttacgattttcttcggaacacaaactggaacggctgaaggtttcgctaaggcacttttcgaagaagcgaaagcgcgatatgaaaaggcagcgtttaaagtgattgatttggatgattatgctgctgatttggatgagtatgcagagaagctgaagaaggaaacatatgctttcttcttcttggctacatatggagatggtgagccaactgataatgctgccaaattttataaatggtttactgagggagacgagaaaggcgtttggcttcaaaaacttcaatatggagtatttggtcttggcaacagacaatatgaacatttcaacaagattggaatagtggttgatgatggtctcaccgagcagggtgcaaaacgcattgttcccgttggtcttggagacgacgatcaatcaattgaagacgatttttcggcatggaaagagttagtgtggcccgaattggatctattgcttcgcgatgaagatgacaaagctgctgcaactccttacacagctgcaatccctgaataccgcgtcgtatttcatgacaaacccgatgcgttttctgatgatcatactcaaaccaatggtcatgctgttcatgatgctcaacatccatgcagatccaatgtggctgttaaaaaagagcttcatactcctgaatccgatcgttcatgcacacatcttgaatttgacatttctcacactggattatcttatgaaactggggatcatgttggtgtatactgtgaaaacctaattgaagtagtggaagaagctgggaaattgttaggattatcaacagatacttatttctcgttacatattgataacgaagatggttcaccacttggtggaccttcattacaacctccttttcctccttgtactttaagaaaagcattgactaattatgcagatctgttaagctctcccaaaaagtcaactttgcttgctctagctgctcatgcttccgatcccactgaagctgatcgtttaagatttcttgcatctcgcgagggcaaggatgaatatgctgaatgggttgttgcaaaccaaagaagtcttcttgaagtcatggaagctttcccgtcagctagaccgccacttggtgttttctttgcagcggttgcaccgcgtttacagcctcgttactactctatttcttcctccccaaagatggaaccaaacaggattcatgttacttgcgcgttggtttatgaaaaaactcccgcaggtcgtatccacaaaggaatctgctcaacctggatgaagaacgctgtacctttgaccgaaagtcaagattgcagttgggcaccgatttttgttagaacatcaaacttcagacttccaattgacccgaaagtcccggttatcatgattggtcctggaaccgggttggctccatttaggggttttcttcaagaaagattggctcttaaagaatccggaaccgaactcgggtcatctattttattcttcggttgtagaaaccgcaaagtggattacatatatgagaatgaactcaacaactttgttgaaaatggtgcgctttctgagcttgatgttgctttctcccgcgatggcccgacgaaagaatacgtgcaacataaaatgacccaaaaggcttctgaaatatggaatatgctttctgagggagcatatttatatgtatgtggtgatgctaaaggcatggctaaagatgtacaccgtacacttcacaccattgtgcaagaacagggaagtttggactcgtctaaagcggagttgtatgtgaagaatctacaaatgtcaggaagatacctccgtgatgtttggtaa

SEQ ID NO: 84 - protein, 709 aa, Stevia rebaudiana
MQSESVEASTIDLMTAVLKDTVIDTANASDNGDSKMPPALAMMFEIRDLLLILTTSVAVLVGCFVVLVWKRSSGKKSGKELEPPKIVVPKRRLEQEVDDGKKKVTIFFGTQTGTAEGFAKALFEEAKARYEKAAFKVIDLDDYAADLDEYAEKLKKETYAFFFLATYGDGEPTDNAAKFYKWFTEGDEKGVWLQKLQYGVFGLGNRQYEHFNKIGIVVDDGLTEQGAKRIVPVGLGDDDQSIEDDFSAWKELVWPELDLLLRDEDDKAAATPYTAAIPEYRVVFHDKPDAFSDDHTQTNGHAVHDAQHPCRSNVAVKKELHTPESDRSCTHLEFDISHTGLSYETGDHVGVYCENLIEVVEEAGKLLGLSTDTYFSLHIDNEDGSPLGGPSLQPPFPPCTLRKALTNYADLLSSPKKSTLLALAAHASDPTEADRLRFLASREGKDEYAEWVVANQRSLLEVMEAFPSARPPLGVFFAAVAPRLQPRYYSISSSPKMEPNRIHVTCALVYEKTPAGRIHKGICSTWMKNAVPLTESQDCSWAPIFVRTSNFRLPIDPKVPVIMIGPGTGLAPFRGFLQERLALKESGTELGSSILFFGCRNRKVDYIYENELNNFVENGALSELDVAFSRDGPTKEYVQHKMTQKASEIWNMLSEGAYLYVCGDAKGMAKDVHRTLHTIVQEQGSLDSSKAELYVKNLQMSGRYLRDVW

SEQ ID NO: 85 - DNA, 2124 bp, Artificial Sequence
  Codon-optimized CPR
atgcaatctaactccgtgaagatttcgccgcttgatctggtaactgcgctgtttagcggcaaggttttggacacatcgaacgcatcggaatcgggagaatctgctatgctgccgactatagcgatgattatggagaatcgtgagctgttgatgatactcacaacgtcggttgctgtattgatcggatgcgttgtcgttttggtgtggcggagatcgtctacgaagaagtcggcgttggagccaccggtgattgtggttccgaagagagtgcaagaggaggaagttgatgatggtaagaagaaagttacggttttcttcggcacccaaactggaacagctgaaggcttcgctaaggcacttgttgaggaagctaaagctcgatatgaaaaggctgtctttaaagtaattgatttggatgattatgctgctgatgacgatgagtatgaggagaaactaaagaaagaatctttggcctttttctttttggctacgtatggagatggtgagccaacagataatgctgccagattttataaatggtttactgagggagatgcgaaaggagaatggcttaataagcttcaatatggagtatttggtttgggtaacagacaatatgaacattttaacaagatcgcaaaagtggttgatgatggtcttgtagaacagggtgcaaagcgtcttgttcctgttggacttggagatgatgatcaatgtattgaagatgacttcaccgcatggaaagagttagtatggccggagttggatcaattacttcgtgatgaggatgacacaactgttgctactccatacacagctgctgttgcagaatatcgcgttgtttttcatgaaaaaccagacgcgctttctgaagattatagttatacaaatggccatgctgttcatgatgctcaacatccatgcagatccaacgtggctgtcaaaaaggaacttcatagtcctgaatctgaccggtcttgcactcatcttgaatttgacatctcgaacaccggactatcatatgaaactggggaccatgttggagtttactgtgaaaacttgagtgaagttgtgaatgatgctgaaagattagtaggattaccaccagacacttactcctccatccacactgatagtgaagacgggtcgccacttggcggagcctcattgccgcctcctttcccgccatgcactttaaggaaagcattgacgtgttatgctgatgttttgagttctcccaagaagtcggctttgcttgcactagctgctcatgccaccgatcccagtgaagctgatagattgaaatttcttgcatcccccgccggaaaggatgaatattctcaatggatagttgcaagccaaagaagtctccttgaagtcatggaagcattcccgtcagctaagccttcacttggtgttttctttgcatctgttgccccgcgcttacaaccaagatactactctatttcttcctcacccaagatggcaccggataggattcatgttacatgtgcattagtctatgagaaaacacctgcaggccgcatccacaaaggagtttgttcaacttggatgaagaacgcagtgcctatgaccgagagtcaagattgcagttgggccccaatatacgtccgaacatccaatttcagactaccatctgaccctaaggtcccggttatcatgattggacctggcactggtttggctccttttagaggtttccttcaagagcggttagctttaaaggaagccggaactgacctcggtttatccattttattcttcggatgtaggaatcgcaaagtggatttcatatatgaaaacgagcttaacaactttgtggagactggtgctctttctgagcttattgttgctttctcccgtgaaggcccgactaaggaatatgtgcaacacaagatgagtgagaaggcttcggatatctggaacttgctttctgaaggagcatatttatacgtatgtggtgatgccaaaggcatggccaaagatgtacatcgaaccctccacacaattgtgcaagaacagggatctcttgactcgtcaaaggcagaactctacgtgaagaatctacaaatgtcaggaagatacctccgtgacgtttggtaa

SEQ ID NO: 86 - protein, 707 aa, Stevia rebaudiana
MQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYSSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 87 - DNA, 2070 bp, Artificial Sequence
  Codon-optimized CPR
atgtcctccaactccgatttggtcagaagattggaatctgttttgggtgtttctttcggtggttctgttactgattccgttgttgttattgctaccacctctattgctttggttatcggtgttttggttttgttgtggagaagatcctctgacagatctagagaagttaagcaattggctgttccaaagccagttactatcgttgaagaagaagatgaattcgaagttgcttctggtaagaccagagtttctattttctacggtactcaaactggtactgctgaaggttttgctaaggctttggctgaagaaatcaaagccagatacgaaaaagctgccgttaaggttattgatttggatgattacacagccgaagatgacaaatacggtgaaaagttgaagaaagaaactatggccttcttcatgttggctacttatggtgatggtgaacctactgataatgctgctagattttacaagtggttcaccgaaggtactgatagaggtgtttggttggaacatttgagatacggtgtattcggtttgggtaacagacaatacgaacacttcaacaagattgccaaggttgttgatgatttgttggttgaacaaggtgccaagagattggttactgttggtttgggtgatgatgatcaatgcatcgaagatgatttctccgcttggaaagaagccttgtggccagaattggatcaattattgcaagatgataccaacaccgtttctactccatacactgctgttattccagaatacagagttgttatccacgatccatctgttacctcttatgaagatccatactctaacatggctaacggtaatgcctcttacgatattcatcatccatgtagagctaacgttgccgtccaaaaagaattgcataagccagaatctgacagaagttgcatccatttggaattcgatattttcgctactggtttgacttacgaaaccggtgatcatgttggtgtttacgctgataattgtgatgatactgtagaagaagccgctaagttgttgggtcaaccattggatttgttgttctccattcataccgataacaacgacggtacttctttgggttcttctttgccaccaccatttccaggtccatgtactttgagaactgctttggctagatatgccgatttgttgaatccaccaaaaaaggctgctttgattgctttagctgctcatgctgatgaaccatctgaagctgaaagattgaagttcttgtcatctccacaaggtaaggacgaatattctaaatgggttgtcggttcccaaagatccttggttgaagttatggctgaatttccatctgctaaaccaccattgggtgtattttttgctgctgttgttcctagattgcaacctagatattactccatctcttccagtccaagatttgctccacatagagttcatgttacttgcgctttggtttatggtccaactccaactggtagaattcacagaggtgtatgttcattctggatgaagaatgttgtcccattggaaaagtctcaaaactgttcttgggccccaattttcatcagacaatctaatttcaagttgccagccgatcattctgttccaatagttatggttggtccaggtactggtttagctccttttagaggtttcttacaagaaagattggccttgaaagaagaaggtgctcaagttggtcctgctttgttgttttttggttgcagaaacagacaaatggacttcatctacgaagtcgaattgaacaactttgtcgaacaaggtgctttgtccgaattgatcgttgctttttcaagagaaggtccatccaaagaatacgtccaacataagatggttgaaaaggcagcttacatgtggaacttgatttctcaaggtggttacttctacgtttgtggtgatgctaaaggtatggctagagatgttcatagaacattgcataccatcgtccaacaagaagaaaaggttgattctaccaaggccgaatccatcgttaagaaattgcaaatggacggtagatacttgagagatgtttggtga

SEQ ID NO: 88 - protein, 689 aa, Rubus suavissimus
MSSNSDLVRRLESVLGVSFGGSVTDSVVVIATTSIALVIGVLVLLWRRSSDRSREVKQLAVPKPVTIVEEEDEFEVASGKTRVSIFYGTQTGTAEGFAKALAEEIKARYEKAAVKVIDLDDYTAEDDKYGEKLKKETMAFFMLATYGDGEPTDNAARFYKWFTEGTDRGVWLEHLRYGVFGLGNRQYEHFNKIAKVVDDLLVEQGAKRLVTVGLGDDDQCIEDDFSAWKEALWPELDQLLQDDTNTVSTPYTAVIPEYRVVIHDPSVTSYEDPYSNMANGNASYDIHHPCRANVAVQKELHKPESDRSCIHLEFDIFATGLTYETGDHVGVYADNCDDTVEEAAKLLGQPLDLLFSIHTDNNDGTSLGSSLPPPFPGPCTLRTALARYADLLNPPKKAALIALAAHADEPSEAERLKFLSSPQGKDEYSKWVVGSQRSLVEVMAEFPSAKPPLGVFFAAVVPRLQPRYYSISSSPRFAPHRVHVTCALVYGPTPTGRIHRGVCSFWMKNVVPLEKSQNCSWAPIFIRQSNFKLPADHSVPIVMVGPGTGLAPFRGFLQERLALKEEGAQVGPALLFFGCRNRQMDFIYEVELNNFVEQGALSELIVAFSREGPSKEYVQHKMVEKAAYMWNLISQGGYFYVCGDAKGMARDVHRTLHTIVQQEEKVDSTKAESIVKKLQMDGRYLRDVW

SEQ ID NO: 89 - DNA, 2079 bp, Artificial Sequence
  Codon-optimized CPR
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 90 - protein, 692 aa, Arabidopsis thaliana [sequence omitted]

SEQ ID NO: 91 - DNA, 2139 bp, Artificial Sequence
  Codon-optimized CPR
atgtcttcctcttcctcttccagtacctctatgattgatttgatggctgctattattaaaggtgaaccagttatcgtctccgacccagcaaatgcctctgcttatgaatcagttgctgcagaattgtcttcaatgttgatcgaaaacagacaattcgccatgatcgtaactacatcaatcgctgttttgatcggttgtattgtcatgttggtatggagaagatccggtagtggtaattctaaaagagtcgaacctttgaaaccattagtaattaagccaagagaagaagaaatagatgacggtagaaagaaagttacaatatttttcggtacccaaactggtacagctgaaggttttgcaaaagccttaggtgaagaagctaaggcaagatacgaaaagactagattcaagatagtcgatttggatgactatgccgctgatgacgatgaatacgaagaaaagttgaagaaagaagatgttgcatttttctttttggcaacctatggtgacggtgaaccaactgacaatgcagccagattctacaaatggtttacagagggtaatgatcgtggtgaatggttgaaaaacttaaagtacggtgttttcggtttgggtaacagacaatacgaacatttcaacaaagttgcaaaggttgtcgacgatattttggtcgaacaaggtgctcaaagattagtccaagtaggtttgggtgacgatgaccaatgtatagaagatgactttactgcctggagagaagctttgtggcctgaattagacacaatcttgagagaagaaggtgacaccgccgttgctaccccatatactgctgcagtattagaatacagagtttccatccatgatagtgaagacgcaaagtttaatgatatcactttggccaatggtaacggttatacagttttcgatgcacaacacccttacaaagctaacgttgcagtcaagagagaattacatacaccagaatccgacagaagttgtatacacttggaatttgatatcgctggttccggtttaaccatgaagttgggtgaccatgtaggtgttttatgcgacaatttgtctgaaactgttgatgaagcattgagattgttggatatgtcccctgacacttattttagtttgcacgctgaaaaagaagatggtacaccaatttccagttctttaccacctccattccctccatgtaacttaagaacagccttgaccagatacgcttgcttgttatcatcccctaaaaagtccgccttggttgctttagccgctcatgctagtgatcctactgaagcagaaagattgaaacacttagcatctccagccggtaaagatgaatattcaaagtgggtagttgaatctcaaagatcattgttagaagttatggcagaatttccatctgccaagcctccattaggtgtcttctttgctggtgtagcacctagattgcaaccaagattctactcaatcagttcttcacctaagatcgctgaaactagaattcatgttacatgtgcattagtctacgaaaagatgccaaccggtagaattcacaagggtgtatgctctacttggatgaaaaatgctgttccttacgaaaaatcagaaaagttgttcttaggtagaccaatcttcgtaagacaatcaaacttcaagttgccttctgattcaaaggttccaataatcatgataggtcctggtacaggtttagccccattcagaggtttcttgcaagaaagattggctttagttgaatctggtgtcgaattaggtccttcagttttgttctttggttgtagaaacagaagaatggatttcatctatgaagaagaattgcaaagattcgtcgaatctggtgcattggccgaattatctgtagctttttcaagagaaggtccaactaaggaatacgttcaacataagatgatggataaggcatccgacatatggaacatgatcagtcaaggtgcttatttgtacgtttgcggtgacgcaaagggtatggccagagatgtccatagatctttgcacacaattgctcaagaacaaggttccatggatagtaccaaagctgaaggtttcgtaaagaacttacaaacttccggtagatacttgagagatgtctggtga

SEQ ID NO: 92 - protein, 712 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 93 - DNA, 1503 bp, Artificial Sequence
  Codon-optimized KAH
atggaagcctcttacctatacatttctattttgcttttactggcatcatacctgttcaccactcaacttagaaggaagagcgctaatctaccaccaaccgtgtttccatcaataccaatcattggacacttatacttactcaaaaagcctctttatagaactttagcaaaaattgccgctaagtacggaccaatactgcaattacaactcggctacagacgtgttctggtgatttcctcaccatcagcagcagaagagtgctttaccaataacgatgtaatcttcgcaaatagacctaagacattgtttggcaaaatagtgggtggaacatcccttggcagtttatcctacggcgatcaatggcgtaatctaaggagagtagcttctatcgaaatcctatcagttcataggttgaacgaatttcatgatatcagagtggatgagaacagattgttaattagaaaacttagaagttcatcttctcctgttactcttataacagtcttttatgctctaacattgaacgtcattatgagaatgatctctggcaaaagatatttcgacagtggggatagagaattggaggaggaaggtaagagatttcgagaaatcttagacgaaacgttgcttctagccggtgcttctaatgttggcgactacttaccaatattgaactggttgggagttaagtctcttgaaaagaaattgatcgctttgcagaaaaagagagatgactttttccagggtttgattgaacaggttagaaaatctcgtggtgctaaagtaggcaaaggtagaaaaacgatgatcgaactcttattatctttgcaagagtcagaacctgagtactatacagatgctatgataagatcttttgtcctaggtctgctggctgcaggtagtgatacttcagcgggcactatggaatgggccatgagcttactggtcaatcacccacatgtattgaagaaagctcaagctgaaatcgatagagttatcggtaataacagattgattgacgagtcagacattggaaatatcccttacatcgggtgtattatcaatgaaactctaagactctatccagcagggccattgttgttcccacatgaaagttctgccgactgcgttatttccggttacaatatacctagaggtacaatgttaatcgtaaaccaatgggcgattcatcacgatcctaaagtctgggatgatcctgaaacctttaaacctgaaagatttcaaggattagaaggaactagagatggtttcaaacttatgccattcggttctgggagaagaggatgtccaggtgaaggtttggcaataaggctgttagggatgacactaggctcagtgatccaatgttttgattgggagagagtaggagatgagatggttgacatgacagaaggtttgggtgtcacacttcctaaggccgttccattagttgccaaatgtaagccacgttccgaaatgactaatctcctatccgaactttaa

SEQ ID NO: 94 - protein, 500 aa, Stevia rebaudiana
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 95 - DNA, 1572 bp, Rubus suavissimus
atggaagtaacagtagctagtagtgtagccctgagcctggtctttattagcatagtagtaagatgggcatggagtgtggtgaattgggtgtggtttaagccgaagaagctggaaagatttttgagggagcaaggccttaaaggcaattcctacaggtttttatatggagacatgaaggagaactctatcctgctcaaacaagcaagatccaaacccatgaacctctccacctcccatgacatagcacctcaagtcaccccttttgtcgaccaaaccgtgaaagcttacggtaagaactcttttaattgggttggccccataccaagggtgaacataatgaatccagaagatttgaaggacgtcttaacaaaaaatgttgactttgttaagccaatatcaaacccacttatcaagttgctagctacaggtattgcaatctatgaaggtgagaaatggactaaacacagaaggattatcaacccaacattccattcggagaggctaaagcgtatgttaccttcatttcaccaaagttgtaatgagatggtcaaggaatgggagagcttggtgtcaaaagagggttcatcatgtgagttggatgtctggccttttcttgaaaatatgtcggcagatgtgatctcgagaacagcatttggaactagctacaaaaaaggacagaaaatctttgaactcttgagagagcaagtaatatatgtaacgaaaggctttcaaagtttttacattccaggatggaggtttctcccaactaagatgaacaagaggatgaatgagattaacgaagaaataaaaggattaatcaggggtattataattgacagagagcaaatcattaaggcaggtgaagaaaccaacgatgacttattaggtgcacttatggagtcaaacttgaaggacattcgggaacatgggaaaaacaacaaaaatgttgggatgagtattgaagatgtaattcaggagtgtaagctgttttactttgctgggcaagaaaccacttcagtgttgctggcttggacaatggttttacttggtcaaaatcagaactggcaagatcgagcaagacaagaggttttgcaagtctttggaagcagcaagccagattttgatggtctagctcaccttaaagtcgtaaccatgattttgcttgaagttcttcgattatacccaccagtcattgaacttattcgaaccattcacaagaaaacacaacttgggaagctctcactaccagaaggagttgaagtccgcttaccaacactgctcattcaccatgacaaggaactgtggggtgatgatgcaaaccagttcaatccagagaggttttcggaaggagtttccaaagcaacaaagaaccgactctcattcttccccttcggagccggtccacgcatttgcattggacagaacttttctatgatggaagcaaagttggccttagcattgatcttgcaacacttcacctttgagctttctccatctcatgcacatgctccttcccatcgtataacccttcaaccacagtatggtgttcgtatcattttacatcgacgttag

SEQ ID NO: 96 - DNA, 1572 bp, Artificial Sequence
  Codon-optimized KAH
atggaagtcactgtcgcctcttctgtcgctttatccttagtcttcatttccattgtcgtcagatgggcttggtccgttgtcaactgggtttggttcaaaccaaagaagttggaaagattcttgagagagcaaggtttgaagggtaattcttatagattcttgtacggtgacatgaaggaaaattctattttgttgaagcaagccagatccaaaccaatgaacttgtctacctctcatgatattgctccacaagttactccattcgtcgatcaaactgttaaagcctacggtaagaactctttcaattgggttggtccaattcctagagttaacatcatgaacccagaagatttgaaggatgtcttgaccaagaacgttgacttcgttaagccaatttccaacccattgattaaattgttggctactggtattgccatttacgaaggtgaaaagtggactaagcatagaagaatcatcaaccctaccttccactctgaaagattgaagagaatgttaccatctttccatcaatcctgtaatgaaatggttaaggaatgggaatccttggtttctaaagaaggttcttcttgcgaattggatgtttggccattcttggaaaatatgtctgctgatgtcatttccagaaccgctttcggtacctcctacaagaagggtcaaaagattttcgaattgttgagagagcaagttatttacgttaccaagggtttccaatccttctacatcccaggttggagattcttgccaactaaaatgaacaagcgtatgaacgagatcaacgaagaaattaaaggtttgatcagaggtattattatcgacagagaacaaattattaaagctggtgaagaaaccaacgatgatttgttgggtgctttgatggagtccaacttgaaggatattagagaacatggtaagaacaacaagaatgttggtatgtctattgaagatgttattcaagaatgtaagttattctacttcgctggtcaagagaccacttctgttttgttagcctggactatggtcttgttaggtcaaaaccaaaattggcaagatagagctagacaagaagttttgcaagtcttcggttcttccaagccagactttgatggtttggcccacttgaaggttgttactatgattttgttagaagttttgagattgtacccaccagtcattgagttaatcagaaccattcataaaaagactcaattgggtaaattatctttgccagaaggtgttgaagtcagattaccaaccttgttgattcaccacgataaggaattatggggtgacgacgctaatcaatttaatccagaaagattttccgaaggtgtttccaaggctaccaaaaaccgtttgtccttcttcccatttggtgctggtccacgtatttgtatcggtcaaaacttttccatgatggaagccaagttggctttggctttaatcttgcaacacttcactttcgaattgtctccatcccatgcccacgctccttctcatagaatcactttacaaccacaatacggtgtcagaatcatcttacacagaagataa

SEQ ID NO: 97 - protein, 523 aa, Rubus suavissimus
MEVTVASSVALSLVFISIVVRWAWSVVNWVWFKPKKLERFLREQGLKGNSYRFLYGDMKENSILLKQARSKPMNLSTSHDIAPQVTPFVDQTVKAYGKNSFNWVGPIPRVNIMNPEDLKDVLTKNVDFVKPISNPLIKLLATGIAIYEGEKWTKHRRIINPTFHSERLKRMLPSFHQSCNEMVKEWESLVSKEGSSCELDVWPFLENMSADVISRTAFGTSYKKGQKIFELLREQVIYVTKGFQSFYIPGWRFLPTKMNKRMNEINEEIKGLIRGIIIDREQIIKAGEETNDDLLGALMESNLKDIREHGKNNKNVGMSIEDVIQECKLFYFAGQETTSVLLAWTMVLLGQNQNWQDRARQEVLQVFGSSKPDFDGLAHLKVVTMILLEVLRLYPPVIELIRTIHKKTQLGKLSLPEGVEVRLPTLLIHHDKELWGDDANQFNPERFSEGVSKATKNRLSFFPFGAGPRICIGQNFSMMEAKLALALILQHFTFELSPSHAHAPSHRITLQPQYGVRIILHRR

SEQ ID NO: 98 - DNA, 1566 bp, Prunus avium
atggaagcatcaagggctagttgtgttgcgctatgtgttgtttgggtgagcatagtaattacattggcatggagggtgctgaattgggtgtggttgaggccaaagaaactagaaagatgcttgagggagcaaggccttacaggcaattcttacaggcttttgtttggagacaccaaggatctctcgaagatgctggaacaaacacaatccaaacccatcaaactctccacctcccatgatatagcgccacgagtcaccccatttttccatcgaactgtgaactctaatggcaagaattcttttgtttggatgggccctataccaagagtgcacatcatgaatccagaagatttgaaagatgccttcaacagacatgatgattttcataagacagtaaaaaatcctatcatgaagtctccaccaccgggcattgtaggcattgaaggtgagcaatgggctaaacacagaaagattatcaacccagcattccatttagagaagctaaagggtatggtaccaatattttaccaaagttgtagcgagatgattaacaaatgggagagcttggtgtccaaagagagttcatgtgagttggatgtgtggccttatcttgaaaattttaccagcgatgtgatttcccgagctgcatttggaagtagctatgaagagggaaggaaaatatttcaactactaagagaggaagcaaaagtttattcggtagctctacgaagtgtttacattccaggatggaggtttctaccaaccaagcagaacaagaagacgaaggaaattcacaatgaaattaaaggcttacttaagggcattataaataaaagggaagaggcgatgaaggcaggggaagccactaaagatgacttactaggaatacttatggagtccaacttcagggaaattcaggaacatgggaacaacaaaaatgctggaatgagtattgaagatgtaattggagagtgtaagttgttttactttgctgggcaagagaccacttcggtgttgcttgtttggacaatgattttactaagccaaaatcaggattggcaagctcgtgcaagagaagaggtcttgaaagtctttggaagcaacatcccaacctatgaagagctaagtcacctaaaagttgtgaccatgattttacttgaagttcttcgattatacccatcagtcgttgcgcttcctcgaaccactcacaagaaaacacagcttggaaaattatcattaccagctggagtggaagtctccttgcccatactgcttgttcaccatgacaaagagttgtggggtgaggatgcaaatgagttcaagccagagaggttttcagagggagtttcaaaggcaacaaagaacaaatttacatacttacctttcggagggggtccaaggatttgcattggacaaaactttgccatggtggaagctaaattggccttggccctgattttacaacactttgcctttgagctttctccatcctatgctcatgctccttctgcagttataacccttcaacctcaatttggtgctcatatcattttgcataaacgttga

SEQ ID NO: 99 - DNA, 1567 bp, Artificial Sequence
  Codon-optimized KAH
atggaagcttctagagcatcttgtgttgctttgtgtgttgtttgggtttccatcgttattactttggcttggagagttttgaattgggtctggttaagaccaaaaaagttggaaagatgcttgagagaacaaggtttgactggtaactcttacagattgttgttcggtgataccaaggacttgtctaagatgttggaacaaactcaatccaagcctatcaagttgtctacctctcatgatattgctccaagagttactccattcttccatagaactgttaactccaacggtaagaactcttttgtttggatgggtccaattccaagagtccatattatgaaccctgaagatttgaaggacgctttcaacagacatgatgatttccataagaccgtcaagaacccaattatgaagtctccaccaccaggtatagttggtattgaaggtgaacaatgggccaaacatagaaagattattaacccagccttccacttggaaaagttgaaaggtatggttccaatcttctaccaatcctgctctgaaatgattaacaagtgggaatccttggtttccaaagaatcttcctgtgaattggatgtctggccatatttggaaaacttcacctccgatgttatttccagagctgcttttggttcttcttacgaagaaggtagaaagatcttccaattattgagagaagaagccaaggtttactccgttgctttgagatctgtttacattccaggttggagattcttgccaactaagcaaaacaaaaagaccaaagaaatccacaacgaaatcaagggtttgttgaagggtatcatcaacaagagagaagaagctatgaaggctggtgaagctacaaaagatgatttgttgggtatcttgatggaatccaacttcagagaaatccaagaacacggtaacaacaagaatgccggtatgtctattgaagatgttatcggtgaatgcaagttgttctactttgctggtcaagaaactacctccgttttgttggtttggaccatgattttgttgtcccaaaatcaagattggcaagctagagctagagaagaagtcttgaaagttttcggttctaacatcccaacctacgaagaattgtctcacttgaaggttgtcactatgatcttgttggaagtattgagattatacccatccgttgttgcattgccaagaactactcataagaaaactcaattgggtaaattgtccttgccagctggtgttgaagtttctttgccaattttgttagtccaccacgacaaagaattgtggggtgaagatgctaatgaattcaagccagaaagattctccgaaggtgtttctaaagctaccaagaacaagttcacttacttgccatttggtggtggtccaagaatatgtattggtcaaaatttcgctatggtcgaagctaaattggctttggctttgatcttgcaacatttcgctttcgaattgtcaccatcttatgctcatgctccatctgctgttattacattgcaaccacaatttggtgcccatatcatcttgcataagagataac

SEQ ID NO: 100 - protein, 521 aa, Prunus avium
MEASRASCVALCVVWVSIVITLAWRVLNWVWLRPKKLERCLREQGLTGNSYRLLFGDTKDLSKMLEQTQSKPIKLSTSHDIAPRVTPFFHRTVNSNGKNSFVWMGPIPRVHIMNPEDLKDAFNRHDDFHKTVKNPIMKSPPPGIVGIEGEQWAKHRKIINPAFHLEKLKGMVPIFYQSCSEMINKWESLVSKESSCELDVWPYLENFTSDVISRAAFGSSYEEGRKIFQLLREEAKVYSVALRSVYIPGWRFLPTKQNKKTKEIHNEIKGLLKGIINKREEAMKAGEATKDDLLGILMESNFREIQEHGNNKNAGMSIEDVIGECKLFYFAGQETTSVLLVWTMILLSQNQDWQARAREEVLKVFGSNIPTYEELSHLKVVTMILLEVLRLYPSVVALPRTTHKKTQLGKLSLPAGVEVSLPILLVHHDKELWGEDANEFKPERFSEGVSKATKNKFTYLPFGGGPRICIGQNFAMVEAKLALALILQHFAFELSPSYAHAPSAVITLQPQFGAHIILHKR

SEQ ID NO: 101 - protein, 517 aa, Prunus mume
ASWVAVLSVVWVSMVIAWAWRVLNWVWLRPKKLEKCLREQGLAGNSYRLLFGDTKDLSKMLEQTQSKPIKLSTSHDIAPHVTPFFHQTVNSYGKNSFVWMGPIPRVHIMNPEDLKDTFNRHDDFHKVVKNPIMKSLPQGIVGIEGEQWAKHRKIINPAFHLEKLKGMVPIFYRSCSEMINKWESLVSKESSCELDVWPYLENFTSDVISRAAFGSSYEEGRKIFQLLREEAKIYTVAMRSVYIPGWRFLPTKQNKKAKEIHNEIKGLLKGIINKREEAMKAGEATKDDLLGILMESNFREIQEHGNNKNAGMSIEDVIGECKLFYFAGQETTSVLLVWTMVLLSQNQDWQARAREEVLQVFGSNIPTYEELSQLKVVTMILLEVLRLYPSVVALPRTTHKKTQLGKLSLPAGVEVSLPILLVHHDKELWGEDANEFKPERFSEGVSKATKNQFTYFPFGGGPRICIGQNFAMMEAKLALSLILRHFALELSPLYAHAPSVTITLQPQYGAHIILHKR

SEQ ID NO: 102 - protein, 521 aa, Prunus mume
MEASRPSCVALSVVLVSIVIAWAWRVLNWVWLRPNKLERCLREQGLTGNSYRLLFGDTKEISMMVEQAQSKPIKLSTTHDIAPRVIPFSHQIVYTYGRNSFVWMGPTPRVTIMNPEDLKDAFNKSDEFQRAISNPIVKSISQGLSSLEGEKWAKHRKIINPAFHLEKLKGMLPTFYQSCSEMINKWESLVFKEGSREMDVWPYLENLTSDVISRAAFGSSYEEGRKIFQLLREEAKFYTIAARSVYIPGWRFLPTKQNKRMKEIHKEVRGLLKGIINKREDAIKAGEAAKGNLLGILMESNFREIQEHGNNKNAGMSIEDVIGECKLFYFAGQETTSVLLVWTLVLLSQNQDWQARAREEVLQVFGTNIPTYDQLSHLKVVTMILLEVLRLYPAVVELPRTTYKKTQLGKFLLPAGVEVSLHIMLAHHDKELWGEDAKEFKPERFSEGVSKATKNQFTYFPFGAGPRICIGQNFAMLEAKLALSLILQHFTFELSPSYAHAPSVTITLHPQFGAHFILHKR

SEQ ID NO: 103 - protein, 514 aa, Prunus mume
CVALSVVLVSIVIAWAWRVLNWVWLRPNKLERCLREQGLTGNSYRLLFGDTKEISMMVEQAQSKPIKLSTTHDIAPRVIPFSHQIVYTYGRNSFVWMGPTPRVTIMNPEDLKDAFNKSDEFQRAISNPIVKSISQGLSSLEGEKWAKHRKIINPAFHLEKLKGMLPTFYQSCSEMINKWESLVFKEGSREMDVWPYLENLTSDVISRAAFGSSYEEGRKIFQLLREEAKFYTIAARSVYIPGWRFLPTKQNKRMKEIHKEVRGLLKGIINKREDAIKAGEAAKGNLLGILMESNFREIQEHGNNKNAGMSIEDVIGECKLFYFAGQETTSVLLVWTLVLLSQNQDWQARAREEVLQVFGTNIPTYDQLSHLKVVTMILLEVLRLYPAVVELPRTTYKKTQLGKFLLPAGVEVSLHIMLAHHDKELWGEDAKEFKPERFSEGVSKATKNQFTYFPFGAGPRICIGQNFAMLEAKLALSLILQHFTFELSPSYAHAPSVTITLHPQFGAHFILHKR

SEQ ID NO: 104 - protein, 418 aa, Prunus persica
MGPIPRVHIMNPEDLKDTFNRHDDFHKVVKNPIMKSLPQGIVGIEGDQWAKHRKIINPAFHLEKLKGMVPIFYQSCSEMINIWKSLVSKESSCELDVWPYLENFTSDVISRAAFGSSYEEGRKIFQLLREEAKVYTVAVRSVYIPGWRFLPTKQNKKTKEIHNEIKGLLKGIINKREEAMKAGEATKDDLLGILMESNFREIQEHGNNKNAGMSIEDVIGECKLFYFAGQETTSVLLVWTMVLLSQNQDWQARAREEVLQVFGSNIPTYEELSHLKVVTMILLEVLRLYPSVVALPRTTHKKTQLGKLSLPAGVEVSLPILLVHHDKELWGEDANEFKPERFSEGVSKATKNQFTYFPFGGGPRICIGQNFAMMEAKLALSLILQHFTFELSPQYSHAPSVTITLQPQYGAHLILHKR

SEQ ID NO: 105 - DNA, 1578 bp, Artificial Sequence
  Codon-optimized KAH
atgggtttgttcccattagaggattcctacgcgctggtctttgaaggactagcaataacactggctttgtactatctactgtctttcatctacaaaacatctaaaaagacatgtacacctcctaaagcatctggtgaaatcattccaattacaggaatcatattgaatctgctatctggctcaagtggtctacctattatcttagcacttgcctctttagcagacagatgtggtcctattttcaccattaggctgggtattaggagagtgctagtagtatcaaattgggaaatcgctaaggagattttcactacccacgatttgatagtttctaatagaccaaaatacttagccgctaagattcttggtttcaattatgtttcattctctttcgctccatacggcccatattgggtcggaatcagaaagattattgctacaaaactaatgtcttcttccagacttcagaagttgcaatttgtaagagtttttgaactagaaaactctatgaaatctatcagagaatcatggaaggagaaaaaggatgaagagggaaaggtattagttgagatgaaaaagtggttctgggaactgaatatgaacatagtgttaaggacagttgctggtaaacaatacactggtacagttgatgatgccgatgcaaagcgtatctccgagttattcagagaatggtttcactacactggcagatttgtcgttggagacgcttttccttttctaggttggttggacctgggcggatacaaaaagacaatggaattagttgctagtagattggactcaatggtcagtaaatggttagatgagcatcgtaaaaagcaagctaacgatgacaaaaaggaggatatggatttcatggatatcatgatctccatgacagaagcaaattcaccacttgaaggatacggcactgatactattatcaagaccacatgtatgactttgattgtttcaggagttgatacaacctcaatcgtacttacttgggccttatcacttttgttaaacaacagagatactttgaaaaaggcacaagaggaattagatatgtgcgtaggtaaaggaagacaagtcaacgagtctgatcttgttaacttgatatacttggaagcagtgcttaaagaggctttaagactttacccagcagcgttcttaggcggaccaagagcattcttggaagattgtactgttgctggttatagaattccaaagggcacctgcttgttgattaacatgtggaaactgcatagagatccaaacatttggagtgatccttgcgaattcaagccagaaagatttttgacacctaatcaaaaggatgttgatgtgatcggtatggatttcgaattgataccatttggtgccggcagaagatattgtccaggtactagattggctttacagatgttgcatatcgtattagcgacattgctgcaaaacttcgaaatgtcaacaccaaacgatgcgccagtcgatatgactgcttctgttggcatgacaaatgccaaagcatcacctttagaagtcttgctatcacctcgtgttaaatggtcctaa

SEQ ID NO: 106 - protein, 522 aa, Stevia rebaudiana
MGLFPLEDSYALVFEGLAITLALYYLLSFIYKTSKKTCTPPKASGEHPITGHLNLLSGSSGLPHLALASLADRCGPIFTIRLGIRRVLVVSNWEIAKEIFTTHDLIVSNRPKYLAAKILGFNYVSFSFAPYGPYWVGIRKIIATKLMSSSRLQKLQFVRVFELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIVLTWALSLLLNNRDTLKKAQEELDMCVGKGRQVNESDLVNLIYLEAVLKEALRLYPAAFLGGPRAFLEDCTVAGYRIPKGTCLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

SEQ ID NO: 107 - DNA, 1431 bp, Artificial Sequence
  Codon-optimized KAH
atgatacaagttttaactccaattctactcttcctcatcttcttcgttttctggaaagtctacaaacatcaaaagactaaaatcaatctaccaccaggttccttcggctggccatttttgggtgaaaccttagccttacttagagcaggctgggattctgagccagaaagattcgtaagagagcgtatcaaaaagcatggatctccacttgttttcaagacatcactatttggagacagattcgctgttctttgcggtccagctggtaataagtttttgttctgcaacgaaaacaaattagtggcatcttggtggccagtccctgtaaggaagttgttcggtaaaagtttactcacaataagaggagatgaagcaaaatggatgagaaaaatgctattgtcttacttgggtccagatgcatttgccacacattatgccgttactatggatgttgtaacacgtagacatattgatgtccattggaggggcaaggaggaagttaatgtatttcaaacagttaagttgtacgcattcgaattagcttgtagattattcatgaacctagatgacccaaaccacatcgcgaaactcggtagtcttttcaacattttcctcaaagggatcatcgagcttcctatagacgttcctggaactagattttactccagtaaaaaggccgcagctgccattagaattgaattgaaaaagctcattaaagctagaaaactcgaattgaaggagggtaaggcgtcttcttcacaggacttgctttctcatctattaacatcacctgatgagaatgggatgttcttgacagaagaggaaatagtcgataacattctacttttgttattcgctggtcacgatacctctgcactatcaataacacttttgatgaaaaccttaggtgaacacagtgatgtgtacgacaaggttttgaaggaacaattagaaatttccaaaacaaaggaggcttgggaatcactaaagtgggaagatatccagaagatgaagtactcatggtcagtaatctgtgaagtcatgagattgaatcctcctgtcatagggacatacagagaggcgttggttgatatcgactatgctggttacactatcccaaaaggatggaagttgcattggtcagctgtttctactcaaagagacgaagccaatttcgaagatgtaactagattcgatccatccagatttgaaggggcaggccctactccattcacatttgtgcctttcggtggaggtcctagaatgtgtttaggcaaagagtttgccaggttagaagtgttagcatttctccacaacattgttaccaactttaagtgggatcttctaatccctgatgagaagatcgaatatgatccaatggctactccagctaagggcttgccaattagacttcatccacaccaagtctaa

SEQ ID NO: 108 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDRFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAAAAIRIELKKLIKARKLELKEGKASSSQDLLSHLLTSPDENGMFLTEEEIVDNILLLLFAGHDTSALSITLLMKTLGEHSDVYDKVLKEQLEISKTKEAWESLKWEDIQKMKYSWSVICEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 109 - DNA, 1578 bp, Artificial Sequence
  Codon-optimized KAH
atggagtctttagtggttcatacagtaaatgctatctggtgtattgtaatcgtcgggattttctcagttggttatcacgtttacggtagagctgtggtcgaacaatggagaatgagaagatcactgaagctacaaggtgttaaaggcccaccaccatccatcttcaatggtaacgtctcagaaatgcaacgtatccaatccgaagctaaacactgctctggcgataacattatctcacatgattattcttcttcattattcccacacttcgatcactggagaaaacagtacggcagaatctacacatactctactggattaaagcaacacttgtacatcaatcatccagaaatggtgaaggagctatctcagactaacacattgaacttgggtagaatcacccatataaccaaaagattgaatcctatcttaggtaacggaatcataacctctaatggtcctcattgggcccatcagcgtagaattatcgcctacgagtttactcatgataagatcaagggtatggttggtttgatggttgagtctgctatgcctatgttgaataagtgggaggagatggtaaagagaggcggagaaatgggatgcgacataagagttgatgaggacttgaaagatgtttcagcagatgtgattgcaaaagcctgtttcggatcctcattttctaaaggtaaggctattttctctatgataagagatttgcttacagctatcacaaagagaagtgttctattcagattcaacggattcactgatatggtctttgggagtaaaaagcatggtgacgttgatatagacgctttagaaatggaattggaatcatccatttgggaaactgtcaaggaacgtgaaatagaatgtaaagatactcacaaaaaggatctgatgcaattgattttggaaggggcaatgcgttcatgtgacggtaacctttgggataaatcagcatatagaagatttgttgtagataattgtaaatctatctacttcgcagggcatgatagtacagctgtctcagtgtcatggtgtttgatgttactggccctaaacccatcatggcaagttaagatccgtgatgaaattctgtcttcttgcaaaaatggtattccagatgccgaaagtatcccaaaccttaaaacagtgactatggttattcaagagacaatgagattataccctccagcaccaatcgtcgggagagaagcctctaaagatatcagattgggcgatctagttgttcctaaaggcgtctgtatatggacactaataccagctttacacagagatcctgagatttggggaccagatgcaaacgatttcaaaccagaaagattttctgaaggaatttcaaaggcttgtaagtatcctcaaagttacattccatttggtctgggtcctagaacatgcgttggtaaaaactttggcatgatggaagtaaaggttcttgtttccctgattgtctccaagttctctttcactctatctcctacctaccaacatagtcctagtcacaaacttttagtagaaccacaacatggggtggtaattagagtggtttaa

SEQ ID NO: 110 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 111 - DNA, 1590 bp, Artificial Sequence
  Codon-optimized KAH
atgtacttcctactacaatacctcaacatcacaaccgttggtgtctttgccacattgtttctctcttattgtttacttctctggagaagtagagcgggtaacaaaaagattgccccagaagctgccgctgcatggcctattatcggccacctccacttacttgcaggtggatcccatcaactaccacatattacattgggtaacatggcagataagtacggtcctgtattcacaatcagaataggcttgcatagagctgtagttgtctcatcttgggaaatggcaaaggaatgttcaacagctaatgatcaagtgtcttcttcaagacctgaactattagcttctaagttgttgggttataactacgccatgtttggtttttcaccatacggttcatactggagagaaatgagaaagatcatctctctcgaattactatctaattccagattggaactattgaaagatgttagagcctcagaagttgtcacatctattaaggaactatacaaattgtgggcggaaaagaagaatgagtcaggattggtttctgtcgagatgaaacaatggttcggagatttgactttaaacgtgatcttgagaatggtggctggtaaaagatacttctccgcgagtgacgcttcagaaaacaaacaggcccagcgttgtagaagagtcttcagagaattcttccatctctccggcttgtttgtggttgctgatgctataccttttcttggatggctcgattggggaagacacgagaagaccttgaaaaagaccgccatagaaatggattccatcgcccaggagtggcttgaggaacatagacgtagaaaagattctggagatgataattctacccaagatttcatggacgttatgcaatctgtgctagatggcaaaaatctaggcggatacgatgctgatacgattaacaaggctacatgcttaactcttatatcaggtggcagtgatactactgtagtttctttgacatgggctcttagtcttgtgttaaacaatagagatactttgaaaaaggcacaggaagagttagacatccaagtcggtaaggaaagattggttaacgagcaagacatcagtaagttagtttacttgcaagcaatagtaaaagagacactcagactttatccaccaggtcctttgggtggtttgagacaattcactgaagattgtacactaggtggctatcacgtttcaaaaggaactagattaatcatgaacttatccaagattcaaaaagatccacgtatttggtctgatcctactgaattccaaccagagagattccttacgactcataaagatgtcgatccacgtggtaaacactttgaattcattccattcggtgcaggaagacgtgcatgtcctggtatcacattcggattacaagtactacatctaacattggcatctttcttgcatgcgtttgaattttcaacaccatcaaatgagcaggttaacatgagagaatcattaggtcttacgaatatgaaatctaccccattagaagttttgatttctccaagactatcccttaattgcttcaaccttatgaaaatttga

SEQ ID NO: 112 - protein, 526 aa, Vitis vinifera
MYFLLQYLNITTVGVFATLFLSYCLLLWRSRAGNKKIAPEAAAAWPIIGHLHLLAGGSHQLPHITLGNMADKYGPVFTIRIGLHRAVVVSSWEMAKECSTANDQVSSSRPELLASKLLGYNYAMFGFSPYGSYWREMRKIISLELLSNSRLELLKDVRASEVVTSIKELYKLWAEKKNESGLVSVEMKQWFGDLTLNVILRMVAGKRYFSASDASENKQAQRCRRVFREFFHLSGLFVVADAIPFLGWLDWGRHEKTLKKTAIEMDSIAQEWLEEHRRRKDSGDDNSTQDFMDVMQSVLDGKNLGGYDADTINKATCLTLISGGSDTTVVSLTWALSLVLNNRDTLKKAQEELDIQVGKERLVNEQDISKLVYLQAIVKETLRLYPPGPLGGLRQFTEDCTLGGYHVSKGTRLIMNLSKIQKDPRIWSDPTEFQPERFLTTHKDVDPRGKHFEFIPFGAGRRACPGITFGLQVLHLTLASFLHAFEFSTPSNEQVNMRESLGLTNMKSTPLEVLISPRLSSCSLYN

SEQ ID NO: 113 - DNA, 1440 bp, Artificial Sequence
  Codon-optimized KAH
atggaacctaacttttacttgtcattactattgttgttcgtgaccttcatttctttaagtctgtttttcatcttttacaaacaaaagtccccattgaatttgccaccagggaaaatgggttaccctatcataggtgaaagtttagaattcctatccacaggctggaagggacatcctgaaaagttcatatttgatagaatgcgtaagtacagtagtgagttattcaagacttctattgtaggcgaatccacagttgtttgctgtggggcagctagtaacaaattcctattctctaacgaaaacaaactggtaactgcctggtggccagattctgttaacaaaatcttcccaacaacttcactggattctaatttgaaggaggaatctataaagatgagaaagttgctgccacagttcttcaaaccagaagcacttcaaagatacgtcggcgttatggatgtaatcgcacaaagacattttgtcactcactgggacaacaaaaatgagatcacagtttatccacttgctaaaagatacactttcttgcttgcgtgtagactgttcatgtctgttgaggatgaaaatcatgtggcgaaattctcagacccattccaactaatcgctgcaggcatcatttcacttcctatcgatcttcctggtactccattcaacaaggccataaaggcttcaaatttcattagaaaagagctgataaagattatcaaacaaagacgtgttgatctggcagagggtacagcatctccaacccaggatatcttgtcacatatgctattaacatctgatgaaaacggtaaatctatgaacgagttgaacattgccgacaagattcttggactattgataggaggccacgatacagcttcagtagcttgcacatttctagtgaagtacttaggagaattaccacatatctacgataaagtctaccaagagcaaatggaaattgccaagtccaaacctgctggggaattgttgaattgggatgacttgaaaaagatgaagtattcatggaatgtggcatgtgaggtaatgagattgtcaccacctttacaaggtggttttagagaggctataactgactttatgtttaacggtttctctattccaaaagggtggaagttatactggtccgccaactctacacacaaaaatgcagaatgtttcccaatgcctgagaaattcgatcctaccagatttgaaggtaatggtccagcgccttatacatttgtaccattcggtggaggccctagaatgtgtcctggaaaggaatacgctagattagaaatcttggttttcatgcataatctggtcaaacgttttaagtgggaaaaggttattccagacgaaaagattattgtcgatccattcccaatcccagctaaagatcttccaatccgtttgtatcctcacaaagcttaa

SEQ ID NO: 114 - protein, 479 aa, Medicago truncatula
MEPNFYLSLLLLFVTFISLSLFFIFYKQKSPLNLPPGKMGYPIIGESLEFLSTGWKGHPEKFIFDRMRKYSSELFKTSIVGESTVVCCGAASNKFLFSNENKLVTAWWPDSVNKIFPTTSLDSNLKEESIKMRKLLPQFFKPEALQRYVGVMDVIAQRHFVTHWDNKNEITVYPLAKRYTFLLACRLFMSVEDENHVAKFSDPFQLIAAGIISLPIDLPGTPFNKAIKASNFIRKELIKIIKQRRVDLAEGTASPTQDILSHMLLTSDENGKSMNELNIADKILGLLIGGHDTASVACTFLVKYLGELPHIYDKVYQEQMEIAKSKPAGELLNWDDLKKMKYSWNVACEVMRLSPPLQGGFREAITDFMFNGFSIPKGWKLYWSANSTHKNAECFPMPEKFDPTRFEGNGPAPYTFVPFGGGPRMCPGKEYARLEILVFMHNLVKRFKWEKVIPDEKIIVDPFPIPAKDLPIRLYPHKA

SEQ ID NO: 115 - DNA, 1116 bp, Artificial Sequence
  Codon-optimized GGPPS
atggcctctgttactttgggttcctggatcgtcgtccaccaccataaccatcaccatccatcatctatcctaactaaatctcgttcaagatcctgtcctattacactaaccaaaccaatctcttttcgttcaaagagaacagtttcctctagtagttctatcgtgtcctctagtgtcgtcactaaggaagacaatctgagacagtctgaaccttcttcctttgatttcatgtcatatatcattactaaggcagaactagtgaataaggctcttgattcagcagttccattaagagagccattgaaaatccatgaagcaatgagatactctcttctagctggcgggaagagagtcagacctgtactctgcatagcagcgtgcgaattagttggtggcgaggaatcaaccgctatgcctgccgcttgtgctgtagaaatgattcatacaatgtcactgatacacgatgatttgccatgtatggataacgatgatctgagaaggggtaagccaactaaccataaggttttcggcgaagatgttgccgtcttagctggtgatgctttgttatctttcgcgttcgaacatttggcatccgcaacatcaagtgatgttgtgtcaccagtaagagtagttagagcagttggagaactggctaaagctattggaactgagggtttagttgcaggtcaagtcgtcgatatctcttccgaaggtcttgatttgaatgatgtaggtcttgaacatctcgaattcatccatcttcacaagacagctgcacttttagaagccagtgcggttctcggcgcaattgttggcggagggagtgatgacgaaattgagagattgaggaagtttgctagatgtataggattactgttccaagtagtagacgatatactagatgtgacaaagtcttccaaagagttgggaaaaacagctggtaaagatttgattgccgacaaattgacctaccctaagattatggggctagaaaaatcaagagaatttgccgagaaactcaatagagaggcgcgtgatcaactgttgggtttcgattctgataaagttgcaccactcttagccttagccaactacatcgcttacagacaaaactaa

SEQ ID NO: 116 - protein, 371 aa, Arabidopsis thaliana
MASVTLGSWIVVHHHNHHHPSSILTKSRSRSCPITLTKPISFRSKRTVSSSSSIVSSSVVTKEDNLRQSEPSSFDFMSYIITKAELVNKALDSAVPLREPLKIHEAMRYSLLAGGKRVRPVLCIAACELVGGEESTAMPAACAVEMIHTMSLIHDDLPCMDNDDLRRGKPTNHKVFGEDVAVLAGDALLSFAFEHLASATSSDVVSPVRVVRAVGELAKAIGTEGLVAGQVVDISSEGLDLNDVGLEHLEFIHLHKTAALLEASAVLGAIVGGGSDDEIERLRKFARCIGLLFQVVDDILDVTKSSKELGKTAGKDLIADKLTYPKIMGLEKSREFAEKLNREARDQLLGFDSDKVAPLLALANYIAYRQN

SEQ ID NO: 117 - protein, 511 aa, Rubus suavissimus
MATLLEHFQAMPFAIPIALAALSWLFLFYIKVSFFSNKSAQAKLPPVPVVPGLPVIGNLLQLKEKKPYQTFTRWAEEYGPIYSIRTGASTMVVLNTTQVAKEAMVTRYLSISTRKLSNALKILTADKCMVAISDYNDFHKMIKRYILSNVLGPSAQKRHRSNRDTLRANVCSRLHSQVKNSPREAVNFRRVFEWELFGIALKQAFGKDIEKPIYVEELGTTLSRDEIFKVLVLDIMEGAIEVDWRDFFPYLRWIPNTRMETKIQRLYFRRKAVMTALINEQKKRIASGEEINCYIDFLLKEGKTLTMDQISMLLWETVIETADTTMVTTEWAMYEVAKDSKRQDRLYQEIQKVCGSEMVTEEYLSQLPYLNAVFHETLRKHSPAALVPLRYAHEDTQLGGYYIPAGTEIAINIYGCNMDKHQWESPEEWKPERFLDPKFDPMDLYKTMAFGAGKRVCAGSLQAMLIACPTIGRLVQEFEWKLRDGEEENVDTVGLTTHKRYPMHAILKPRS

SEQ ID NO: 118-125 - skipped (8 entries)

SEQ ID NO: 126 - DNA, 1476 bp, Arabidopsis thaliana
atggcatcggaatttcgtcctcctcttcattttgttctcttccctttcatggctcaaggccacatgatcccaatggtagatattgcaaggctcctggctcagcgcggggtgactataaccattgtcactacacctcaaaacgcaggccggttcaagaacgttcttagccgggctatccaatccggcttgcccatcaatctcgtgcaagtaaagtttccatctcaagaatcgggttcaccggaaggacaggagaatttggacttgctcgattcattgggggcttcattaaccttcttcaaagcatttagcctgctcgaggaaccagtcgagaagctcttgaaagagattcaacctaggccaaactgcataatcgctgacatgtgtttgccttatacaaacagaattgccaagaatcttggtataccaaaaatcatctttcatggcatgtgttgcttcaatcttctttgtacgcacataatgcaccaaaaccacgagttcttggaaactatagagtctgacaaggaatacttccccattcctaatttccctgacagagttgagttcacaaaatctcagcttccaatggtattagttgctggagattggaaagacttccttgacggaatgacagaaggggataacacttcttatggtgtgattgttaacacgtttgaagagctcgagccagcttatgttagagactacaagaaggttaaagcgggtaagatatggagcatcggaccggtttccttgtgcaacaagttaggagaagaccaagctgagaggggaaacaaggcggacattgatcaagacgagtgtattaaatggcttgattctaaagaagaagggtcggtgctatatgtttgccttggaagtatatgcaatcttcctctgtctcagctcaaagagctcggcttaggcctcgaggaatcccaaagacctttcatttgggtcataagaggttgggagaagtataacgagttacttgaatggatctcagagagcggttataaggaaagaatcaaagaaagaggccttctcataacaggatggtcgcctcaaatgcttatccttacacatcctgccgttggaggattcttgacacattgtggatggaactctactcttgaaggaatcacttcaggcgttccattactcacgtggccactgtttggagaccaattctgcaatgagaaattggcggtgcagatactaaaagccggtgtgagagctggggttgaagagtccatgagatggggagaagaggagaaaataggagtactggtggataaagaaggagtaaagaaggcagtggaggaattgatgggtgatagtaatgatgctaaggagagaagaaaaagagtgaaagagcttggagaattagctcacaaggctgtggaagaaggaggctcttctcattccaacatcacattcttgctacaagacataatgcaattagaacaacccaagcgctag

SEQ ID NO: 127 - protein, 491 aa, Arabidopsis thaliana
MASEFRPPLHFVLFPFMAQGHMIPMVDIARLLAQRGVTITIVTTPQNAGRFKNVLSRAIQSGLPINLVQVKFPSQESGSPEGQENLDLLDSLGASLTFFKAFSLLEEPVEKLLKEIQPRPNCIIADMCLPYTNRIAKNLGIPKIIFHGMCCFNLLCTHIMHQNHEFLETIESDKEYFPIPNFPDRVEFTKSQLPMVLVAGDWKDFLDGMTEGDNTSYGVIVNTFEELEPAYVRDYKKVKAGKIWSIGPVSLCNKLGEDQAERGNKADIDQDECIKWLDSKEEGSVLYVCLGSICNLPLSQLKELGLGLEESQRPFIWVIRGWEKYNELLEWISESGYKERIKERGLLITGWSPQMLILTHPAVGGFLTHCGWNSTLEGITSGVPLLTWPLFGDQFCNEKLAVQILKAGVRAGVEESMRWGEEEKIGVLVDKEGVKKAVEELMGDSNDAKERRKRVKELGELAHKAVEEGGSSHSNITFLLQDIMQLEQPKR

SEQ ID NO: 128-131 - skipped (4 entries)

SEQ ID NO: 132 - DNA, 1491 bp, Arabidopsis thaliana
atggctacggaaaaaacccaccaatttcatccttctcttcactttgtcctcttccctttcatggctcaaggccacatgattcccatgattgatattgcaagactcttggctcagcgtggtgtgaccataacaattgtcacgacacctcacaacgcagcaaggtttaagaatgtcctaaaccgagcgatcgagtctggcttggccatcaacatactgcatgtgaagtttccatatcaagagtttggtttgccagaaggaaaagagaatatagattcgttagactcaacggagttgatggtacctttcttcaaagcggtgaacttgcttgaagatccggtcatgaagctcatggaagagatgaaacctagacctagctgtctaatttctgattggtgtttgccttatacaagcataatcgccaagaacttcaatataccaaagatagttttccacggcatgggttgctttaatcttttgtgtatgcatgttctacgcagaaacttagagatcctagagaatgtaaagtcggatgaagagtatttcttggttcctagttttcctgatagagttgaatttacaaagcttcaacttcctgtgaaagcaaatgcaagtggagattggaaagagataatggatgaaatggtaaaagcagaatacacatcctatggtgtgatcgtcaacacatttcaggagttggagccaccttatgtcaaagactacaaagaggcaatggatggaaaagtatggtccattggacccgtttccttgtgtaacaaggcaggtgcagacaaagctgagaggggaagcaaggccgccattgatcaagatgagtgtcttcaatggcttgattctaaagaagaaggttcggtgctctatgtttgccttggaagtatatgtaatcttcctttgtctcagctcaaggagctggggctaggccttgaggaatctcgaagatcttttatttgggtcataagaggttcggaaaagtataaagaactatttgagtggatgttggagagcggttttgaagaaagaatcaaagagagaggacttctcattaaagggtgggcacctcaagtccttatcctttcacatccttccgttggaggattcctgacacactgtggatggaactcgactctcgaaggaatcacctcaggcattccactgatcacttggccgctgtttggagaccaattctgcaaccaaaaactggtcgttcaagtactaaaagccggtgtaagtgccggggttgaagaagtcatgaaatggggagaagaagataaaataggagtgttagtggataaagaaggagtgaaaaaggctgtggaagaattgatgggtgatagtgatgatgcaaaagagaggagaagaagagtcaaagagcttggagaattagctcacaaagctgtggaaaaaggaggctcttctcattctaacatcacactcttgctacaagacataatgcaactagcacaattcaagaattga

SEQ ID NO: 133 - protein, 496 aa, Arabidopsis thaliana
MATEKTHQFHPSLHFVLFPFMAQGHMIPMIDIARLLAQRGVTITIVTTPHNAARFKNVLNRAIESGLAINILHVKFPYQEFGLPEGKENIDSLDSTELMVPFFKAVNLLEDPVMKLMEEMKPRPSCLISDWCLPYTSIIAKNFNIPKIVFHGMGCFNLLCMHVLRRNLEILENVKSDEEYFLVPSFPDRVEFTKLQLPVKANASGDWKEIMDEMVKAEYTSYGVIVNTFQELEPPYVKDYKEAMDGKVWSIGPVSLCNKAGADKAERGSKAAIDQDECLQWLDSKEEGSVLYVCLGSICNLPLSQLKELGLGLEESRRSFIWVIRGSEKYKELFEWMLESGFEERIKERGLLIKGWAPQVLILSHPSVGGFLTHCGWNSTLEGITSGIPLITWPLFGDQFCNQKLVVQVLKAGVSAGVEEVMKWGEEDKIGVLVDKEGVKKAVEELMGDSDDAKERRRRVKELGELAHKAVEKGGSSHSNITLLLQDIMQLAQFKN

SEQ ID NO: 134 - DNA, 1488 bp, Arabidopsis thaliana
atggtttccgaaacaaccaaatcttctccacttcactttgttctcttccctttcatggctcaaggccacatgattcccatggttgatattgcaaggctcttggctcagcgtggtgtgatcataacaattgtcacgacgcctcacaatgcagcgaggttcaagaatgtcctaaaccgtgccattgagtctggcttgcccatcaacttagtgcaagtcaagtttccatatctagaagctggtttgcaagaaggacaagagaatatcgattctcttgacacaatggagcggatgatacctttctttaaagcggttaactttctcgaagaaccagtccagaagctcattgaagagatgaaccctcgaccaagctgtctaatttctgatttttgtttgccttatacaagcaaaatcgccaagaagttcaatatcccaaagatcctcttccatggcatgggttgcttttgtcttctgtgtatgcatgttttacgcaagaaccgtgagatcttggacaatttaaagtcagataaggagcttttcactgttcctgattttcctgatagagttgaattcacaagaacgcaagttccggtagaaacatatgttccagctggagactggaaagatatctttgatggtatggtagaagcgaatgagacatcttatggtgtgatcgtcaactcatttcaagagctcgagcctgcttatgccaaagactacaaggaggtaaggtccggtaaagcatggaccattggacccgtttccttgtgcaacaaggtaggagccgacaaagcagagaggggaaacaaatcagacattgatcaagatgagtgccttaaatggctcgattctaagaaacatggctcggtgctttacgtttgtcttggaagtatctgtaatcttcctttgtctcaactcaaggagctgggactaggcctagaggaatcccaaagacctttcatttgggtcataagaggttgggagaagtacaaagagttagttgagtggttctcggaaagcggctttgaagatagaatccaagatagaggacttctcatcaaaggatggtcccctcaaatgcttatcctttcacatccatcagttggagggttcctaacacactgtggttggaactcgactcttgaggggataactgctggtctaccgctacttacatggccgctattcgcagaccaattctgcaatgagaaattggtcgttgaggtactaaaagccggtgtaagatccggggttgaacagcctatgaaatggggagaagaggagaaaataggagtgttggtggataaagaaggagtgaagaaggcagtggaagaattaatgggtgagagtgatgatgcaaaagagagaagaagaagagccaaagagcttggagattcagctcacaaggctgtggaagaaggaggctcttctcattctaacatctctttcttgctacaagacataatggaactggcagaacccaataattga

SEQ ID NO: 135 - protein, 495 aa, Arabidopsis thaliana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN

SEQ ID NO: 136 - DNA, 1488 bp, Arabidopsis thaliana
atggctttcgaaaaaaacaacgaaccttttcctcttcactttgttctcttccctttcatggctcaaggccacatgattcccatggttgatattgcaaggctcttggctcagcgaggtgtgcttataacaattgtcacgacgcctcacaatgcagcaaggttcaagaatgtcctaaaccgtgccattgagtctggtttgcccatcaacctagtgcaagtcaagtttccatatcaagaagctggtctgcaagaaggacaagaaaatatggatttgcttaccacgatggagcagataacatctttctttaaagcggttaacttactcaaagaaccagtccagaaccttattgaagagatgagcccgcgaccaagctgtctaatctctgatatgtgtttgtcgtatacaagcgaaatcgccaagaagttcaaaataccaaagatcctcttccatggcatgggttgcttttgtcttctgtgtgttaacgttctgcgcaagaaccgtgagatcttggacaatttaaagtctgataaggagtacttcattgttccttattttcctgatagagttgaattcacaagacctcaagttccggtggaaacatatgttcctgcaggctggaaagagatcttggaggatatggtagaagcggataagacatcttatggtgttatagtcaactcatttcaagagctcgaacctgcgtatgccaaagacttcaaggaggcaaggtctggtaaagcatggaccattggacctgtttccttgtgcaacaaggtaggagtagacaaagcagagaggggaaacaaatcagatattgatcaagatgagtgccttgaatggctcgattctaaggaaccgggatctgtgctctacgtttgccttggaagtatttgtaatcttcctctgtctcagctccttgagctgggactaggcctagaggaatcccaaagacctttcatctgggtcataagaggttgggagaaatacaaagagttagttgagtggttctcggaaagcggctttgaagatagaatccaagatagaggacttctcatcaaaggatggtcccctcaaatgcttatcctttcacatccttctgttggagggttcttaacgcactgcggatggaactcgactcttgaggggataactgctggtctaccaatgcttacatggccactatttgcagaccaattctgcaacgagaaactggtcgtacaaatactaaaagtcggtgtaagtgccgaggttaaagaggtcatgaaatggggagaagaagagaagataggagtgttggtggataaagaaggagtgaagaaggcagtggaagaactaatgggtgagagtgatgatgcaaaagagagaagaagaagagccaaagagcttggagaatcagctcacaaggctgtggaagaaggaggctcctctcattctaatatcactttcttgctacaagacataatgcaactagcacagtccaataattga

SEQ ID NO: 137 - protein, 495 aa, Arabidopsis thaliana
MAFEKNNEPFPLHFVLFPFMAQGHMIPMVDIARLLAQRGVLITIVTTPHNAARFKNVLNRAIESGLPINLVQVKFPYQEAGLQEGQENMDLLTTMEQITSFFKAVNLLKEPVQNLIEEMSPRPSCLISDMCLSYTSEIAKKFKIPKILFHGMGCFCLLCVNVLRKNREILDNLKSDKEYFIVPYFPDRVEFTRPQVPVETYVPAGWKEILEDMVEADKTSYGVIVNSFQELEPAYAKDFKEARSGKAWTIGPVSLCNKVGVDKAERGNKSDIDQDECLEWLDSKEPGSVLYVCLGSICNLPLSQLLELGLGLEESQRPFIWVIRGWEKYKELVEWFSESGFEDRIQDRGLLIKGWSPQMLILSHPSVGGFLTHCGWNSTLEGITAGLPMLTWPLFADQFCNEKLVVQILKVGVSAEVKEVMKWGEEEKIGVLVDKEGVKKAVEELMGESDDAKERRRRAKELGESAHKAVEEGGSSHSNITFLLQDIMQLAQSNN

SEQ ID NO: 138 - DNA, 1473 bp, Arabidopsis thaliana
atgtgttctcatgatcctcttcacttcgtcgtaataccctttatggcccaaggccatatgatcccattggtcgacatctctaggctcttgtcccagcgccaaggcgtgactgtctgcatcatcacaactactcaaaatgtagccaagatcaagacttcactctcattttcctctttgtttgcgactatcaacatcgttgaagttaagtttctgtctcaacaaacgggtttgccagaagggtgcgagagtttagatatgttggcttcaatgggcgatatggtgaagttctttgatgctgccaactcacttgaggagcaagttgagaaagctatggaagagatggttcagccgcggccaagctgcatcattggagacatgagccttcctttcacttcaagacttgccaagaaattcaagatccccaaacttatcttccatgggttttcttgtttcagcctcatgtctatacaagtggttcgagaaagcgggatcttgaaaatgatagaatcaaacgacgagtattttgatttgcccggcttgcctgacaaagttgagttcacgaaacctcaggtctctgtgttgcaacctgttgaaggaaatatgaaagagagtacggccaagattattgaagctgataatgactcttatggtgttattgtgaacacttttgaagagttagaggttgattatgcaagagaatataggaaagcaagggctggaaaagtttggtgcgttggacctgtttccttgtgcaataggttagggttagacaaagctaaaagaggagataaggcttctattggtcaagaccaatgtcttcaatggcttgactctcaagaaactggttcagtgctctacgtttgccttggaagtctatgtaatcttcccttggctcagctcaaagagctgggactaggccttgaggcatctaataaacctttcatatgggttataagagaatggggaaaatatggagatttagcaaattggatgcaacaaagcggatttgaagagcggatcaaagatagaggactggtgatcaaaggttgggcgccgcaagttttcatcctctcacacgcatccattggagggtttttgactcactgtggatggaactcgacactagaaggaattactgcaggagttccattattgacatggcctttgtttgctgaacaattcttgaatgagaagttagttgtgcagatactaaaagcagggttaaagataggagtagagaaattgatgaaatatggaaaagaagaggagataggagcgatggtgagcagagaatgtgtgagaaaagctgtggatgagctaatgggtgatagtgaagaagcagaagagagaagaagaaaagttacagaacttagtgacttggcaaataaggctttggaaaaaggaggatcttcagattctaatatcacattgctcattcaagatattatggagcaatcacaaaatcaattctag

SEQ ID NO: 139 - protein, 490 aa, Arabidopsis thaliana
MCSHDPLHFVVIPFMAQGHMIPLVDISRLLSQRQGVTVCIITTTQNVAKIKTSLSFSSLFATINIVEVKFLSQQTGLPEGCESLDMLASMGDMVKFFDAANSLEEQVEKAMEEMVQPRPSCIIGDMSLPFTSRLAKKFKIPKLIFHGFSCFSLMSIQVVRESGILKMIESNDEYFDLPGLPDKVEFTKPQVSVLQPVEGNMKESTAKIIEADNDSYGVIVNTFEELEVDYAREYRKARAGKVWCVGPVSLCNRLGLDKAKRGDKASIGQDQCLQWLDSQETGSVLYVCLGSLCNLPLAQLKELGLGLEASNKPFIWVIREWGKYGDLANWMQQSGFEERIKDRGLVIKGWAPQVFILSHASIGGFLTHCGWNSTLEGITAGVPLLTWPLFAEQFLNEKLVVQILKAGLKIGVEKLMKYGKEEEIGAMVSRECVRKAVDELMGDSEEAEERRRKVTELSDLANKALEKGGSSDSNITLLIQDIMEQSQNQF

SEQ ID NO: 140 - DNA, 1488 bp, Stevia rebaudiana
atgtcgccaaaaatggtggcaccaccaaccaaccttcattttgttttgtttcctcttatggctcaaggccatctggtacccatggtcgacatcgctcgaatcttagcccaacgtggtgcaacggtcaccataatcaccacaccctaccatgccaaccgggtcagaccggttatctcccgagccatcgcgaccaatctcaagatccagctactcgaactccaactgcggtcaaccgaagccggtttacccgaagggtgcgaaagcttcgaccaacttccgtcattcgagtactggaaaaatatttcaaccgctatcgatttgttacaacaacccgctgaagatttgctccgagaactttcaccaccacccgattgcatcatatcggactttttgttcccgtggaccaccgatgtggctcgacggttaaacatcccccggctcgtgttcaatggaccgggctgcttttatctcttgtgcatccatgttgcgatcacttccaacattttgggagagaatgaaccggtcagtagtaataccgagcgcgttgtgctgcccggtttacctgaccggatcgaagtcactaaacttcagatcgtcggttcgtcgagaccagccaacgtagacgaaatgggctcgtggcttcgagccgtagaagctgagaaagcttcattcgggatagtggttaatactttcgaagagcttgaaccggagtacgttgaagaatacaaaacggttaaagataagaagatgtggtgtatcggcccggtttcgttatgcaacaaaaccgggccggatttagccgagcgaggaaacaaagctgcaataaccgaacacaactgcttaaaatggctcgatgagagaaaactggggtccgtgttatacgtttgtttaggtagccttgcacgcatttctgccgcacaagcaatcgagctcgggttaggactcgagtccataaaccgtccctttatatggtgcgtaagaaacgaaaccgatgagctcaaaacatggtttttggatgggtttgaagaaagggttagagatcgcgggttgatcgttcatggttgggcgccacaggttttgatactgtcgcacccaaccattggcggtttcttaacccattgcggttggaactcgactattgaatcgattaccgcgggtgttccaatgatcacgtggccattttttgcggaccagtttttgaatgaagcttttatagttgaagttttgaagattggagttaggattggtgttgagagggcttgtttgtttggggaagaagataaggttggagtgttggtgaagaaggaggatgtgaagaaggctgttgaatgcttgatggatgaagatgaagatggtgatcagagaagaaagagggtgattgagcttgcaaaaatggcgaagattgcaatggcggaaggtggatcttcttatgaaaatgtatcgtcgttgattcgagatgtgactgaaacagttagagcaccacattag

SEQ ID NO: 141 - protein, 495 aa, Stevia rebaudiana
MSPKMVAPPTNLHFVLFPLMAQGHLVPMVDIARILAQRGATVTIITTPYHANRVRPVISRAIATNLKIQLLELQLRSTEAGLPEGCESFDQLPSFEYWKNISTAIDLLQQPAEDLLRELSPPPDCIISDFLFPWTTDVARRLNIPRLVFNGPGCFYLLCIHVAITSNILGENEPVSSNTERVVLPGLPDRIEVTKLQIVGSSRPANVDEMGSWLRAVEAEKASFGIVVNTFEELEPEYVEEYKTVKDKKMWCIGPVSLCNKTGPDLAERGNKAAITEHNCLKWLDERKLGSVLYVCLGSLARISAAQAIELGLGLESINRPFIWCVRNETDELKTWFLDGFEERVRDRGLIVHGWAPQVLILSHPTIGGFLTHCGWNSTIESITAGVPMITWPFFADQFLNEAFIVEVLKIGVRIGVERACLFGEEDKVGVLVKKEDVKKAVECLMDEDEDGDQRRKRVIELAKMAKIAMAEGGSSYENVSSLIRDVTETVRAPH

SEQ ID NO: 142 - DNA, 1371 bp, Arabidopsis thaliana
atgggagagaaagcgaaagcaaatgtgttagtcttctcatttccgatacaaggtcacataaaccctctcctccaattctcaaaacgcctactctctaaaaacgtcaacgtcacattcctcaccacttcctccacccacaactccatcctccgccgtgccatcaccggcggagccactgctcttcctctctcttttgtccccattgacgatggattcgaggaagatcacccatctacggacacatctcccgactacttcgcaaagttccaagaaaacgtatctcgaagcctctcagagcttatctcctcgatggacccaaaaccaaacgccgtcgtttacgactcgtgcctgccttatgtcctcgacgtttgccggaaacatcctggcgttgctgcggcgtcgtttttcactcagtcctccaccgtgaacgcgacctatattcatttcttgcgtggagagtttaaggagtttcaaaatgatgtcgttttgcctgcaatgcctccgctgaagggtaatgacttaccggtgtttctgtacgataacaatctctgccggccgttgtttgagctcattagtagccagttcgtgaatgttgacgacattgacttcttcttggttaactctttcgacgaactcgaagtcgaggtgctacaatggatgaaaaaccaatggccggtcaagaacataggaccgatgattccatcaatgtacttagacaaacgattagcaggtgacaaagactacggaatcaacctcttcaatgcccaagtcaacgaatgccttgattggcttgactcaaaaccgcccggttcagtgatctacgtgtcttttggaagcttggccgtcttaaaagacgatcaaatgatagaagtcgcggctggtctaaaacaaactggccataacttcttatgggttgttagagaaactgaaacaaagaagcttccaagcaattacatagaggacatttgtgacaagggattgatagtgaattggagtcctcaattacaagttcttgcacataaatcaatcggttgtttcatgactcattgcgggtggaattcgactttagaggcattgagcttaggagttgctttgataggaatgccggcttatagcgaccagccgactaatgctaagtttattgaagatgtgtggaaggttggggttagggttaaggcagatcaaaatgggtttgttccgaaggaagagattgtgagatgtgttggagaagttatggaagatatgtcggagaaagggaaggagattagaaaaaatgctcggaggttgatggagtttgcaagggaagctttgtctgatggaggaaattctgataagaatattgatgagtttgttgctaaaattgtgaggtaa

SEQ ID NO: 143 - protein, 456 aa, Arabidopsis thaliana
MGEKAKANVLVFSFPIQGHINPLLQFSKRLLSKNVNVTFLTTSSTHNSILRRAITGGATALPLSFVPIDDGFEEDHPSTDTSPDYFAKFQENVSRSLSELISSMDPKPNAVVYDSCLPYVLDVCRKHPGVAAASFFTQSSTVNATYIHFLRGEFKEFQNDVVLPAMPPLKGNDLPVFLYDNNLCRPLFELISSQFVNVDDIDFFLVNSFDELEVEVLQWMKNQWPVKNIGPMIPSMYLDKRLAGDKDYGINLFNAQVNECLDWLDSKPPGSVIYVSFGSLAVLKDDQMIEVAAGLKQTGHNFLWVVRETETKKLPSNYIEDICDKGLIVNWSPQLQVLAHKSIGCFMTHCGWNSTLEALSLGVALIGMPAYSDQPTNAKFIEDVWKVGVRVKADQNGFVPKEEIVRCVGEVMEDMSEKGKEIRKNARRLMEFAREALSDGGNSDKNIDEFVAKIVR

SEQ ID NO: 144 - DNA, 1410 bp, Arabidopsis thaliana
atggcgccaccgcattttctactggtaacgtttccggcgcaaggtcacgtgaacccatctctccgttttgctcgtcggctcatcaaaagaaccggcgcacgtgtcactttcgtcacttgtgtctccgtcttccacaactccatgatcgcaaaccacaacaaagtcgaaaatctctctttccttactttctccgacggtttcgacgatggaggcatttccacctacgaagaccgtcagaaaaggtcggtgaatctcaaggttaacggcgataaggcactatcggatttcatcgaagctactaagaatggtgactctcccgtgacttgcttgatctacacgattcttctcaattgggctccaaaagtagcacgtagatttcaacttccctccgctcttctctggatccaaccggctttggttttcaacatctattacactcatttcatgggaaacaagtccgttttcgagttacctaatctgtcttctctggaaatcagagatcttccatctttcctcacaccttccaacacaaacaaaggcgcatacgatgcgtttcaagaaatgatggagtttctcataaaagaaaccaaaccgaaaattctcatcaacactttcgattcgctggaaccagaggccttaacggctttcccgaatatcgatatggtggcggttggtcctttacttcccacggagattttctcaggaagcaccaacaaatcagttaaagatcaaagtagtagttatacactttggctagactcgaaaacagagtcctctgttatttacgtttcctttggaacaatggttgagttgtccaagaaacagatagaggaactagcgagagcactcatagaagggaaacgaccgtttttgtgggttataactgataaatccaacagagaaacgaaaacagaaggagaagaagagacagagattgagaagatagctggattcagacacgagcttgaagaggttgggatgattgtgtcgtggtgttcgcagatagaggttttaagtcaccgagccgtaggttgttttgtgactcattgtgggtggagctcgacgctggagagtttggttcttggcgttccggttgtggcgtttccgatgtggtcggatcaaccgacgaacgcgaagctactggaagaaagttggaagactggtgtgagggtaagagagaacaaggatggtttggtggagagaggagagatcaggaggtgtttggaagccgtgatggaggagaagtcggtggagttgagggaaaacgcaaagaaatggaagcgtttagcgatggaagcgggtagagaaggaggatcttcggataagaacatggaggcttttgtggaggatatttgtggagaatctcttattcaaaacttgtgtgaagcagaggaggtaaaagtacgctag

SEQ ID NO: 145 - protein, 469 aa, Arabidopsis thaliana
MAPPHFLLVTFPAQGHVNPSLRFARRLIKRTGARVTFVTCVSVFHNSMIANHNKVENLSFLTFSDGFDDGGISTYEDRQKRSVNLKVNGDKALSDFIEATKNGDSPVTCLIYTILLNWAPKVARRFQLPSALLWIQPALVFNIYYTHFMGNKSVFELPNLSSLEIRDLPSFLTPSNTNKGAYDAFQEMMEFLIKETKPKILINTFDSLEPEALTAFPNIDMVAVGPLLPTEIFSGSTNKSVKDQSSSYTLWLDSKTESSVIYVSFGTMVELSKKQIEELARALIEGKRPFLWVITDKSNRETKTEGEEETEIEKIAGFRHELEEVGMIVSWCSQIEVLSHRAVGCFVTHCGWSSTLESLVLGVPVVAFPMWSDQPTNAKLLEESWKTGVRVRENKDGLVERGEIRRCLEAVMEEKSVELRENAKKWKRLAMEAGREGGSSDKNMEAFVEDICGESLIQNLCEAEEVKVR

SEQ ID NO: 146 - DNA, 1425 bp, Gardenia jasminoides
atggttcaacaaagacacgttttgttgattacctatccagctcaaggtcatattaacccagctttacaattcgcccaaagattattgagaatgggtatccaagttaccttggctacttctgtttatgccttgtccagaatgaagaagtcatctggttctactccaaagggtttgacttttgctactttctctgatggttacgatgatggttttagacctaagggtgttgatcacaccgaatatatgtcatctttggctaagcaaggttccaacactttgagaaacgttattaacacctctgctgatcaaggttgtccagttacttgtttggtttacactttgttgttgccatgggctgctactgttgctagagaatgtcatattccatctgccttgttgtggattcaaccagttgctgttatggacatctattactactacttcagaggttacgaagatgacgtcaagaacaattctaatgatccaacctggtccattcaatttccaggtttgccatctatgaaggctaaagatttgccttcctttatcttgccatcctccgataatatctactcttttgctttgccaaccttcaagaagcaattggaaactttggacgaagaagaaagaccaaaggttttggttaataccttcgatgctttggaaccacaagccttgaaagctattgaatcttacaacttgattgccatcggtccattgactccatctgcttttttggatggtaaagatccatccgaaacatccttttctggtgacttgtttcaaaagtccaaggactacaaagaatggttgaactctagaccagcaggttctgttgtttacgtttcttttggttccttgttgaccttgccaaagcaacaaatggaagaaattgctagaggtttgttgaagtctggtagaccatttttgtgggttatcagagctaaagaaaacggtgaagaagaaaaagaagaagatagattgatctgcatggaagaattggaagaacaaggtatgatagttccatggtgctcccaaattgaagttttgactcatccatctttgggttgcttcgttactcattgtggttggaatagtactttggaaaccttggtttgtggtgttccagttgttgcatttccacattggaccgatcaaggtactaatgccaaattgattgaagatgtttgggaaaccggtgttagagttgttccaaatgaagatggtactgtcgaatctgacgaaatcaagagatgtatcgaaaccgttatggatgatggtgaaaaaggtgtcgaattgaagagaaatgccaagaagtggaaagaattggctagagaagctatgcaagaagatggttcttctgacaagaatttgaaggctttcgttgaagatgctggtaaaggttatcaagccgaatctaactga

SEQ ID NO: 147 - protein, 474 aa, Gardenia jasminoides
MVQQRHVLLITYPAQGHINPALQFAQRLLRMGIQVTLATSVYALSRMKKSSGSTPKGLTFATFSDGYDDGFRPKGVDHTEYMSSLAKQGSNTLRNVINTSADQGCPVTCLVYTLLLPWAATVARECHIPSALLWIQPVAVMDIYYYYFRGYEDDVKNNSNDPTWSIQFPGLPSMKAKDLPSFILPSSDNIYSFALPTFKKQLETLDEEERPKVLVNTFDALEPQALKAIESYNLIAIGPLTPSAFLDGKDPSETSFSGDLFQKSKDYKEWLNSRPAGSVVYVSFGSLLTLPKQQMEEIARGLLKSGRPFLWVIRAKENGEEEKEEDRLICMEELEEQGMIVPWCSQIEVLTHPSLGCFVTHCGWNSTLETLVCGVPVVAFPHWTDQGTNAKLIEDVWETGVRVVPNEDGTVESDEIKRCIETVMDDGEKGVELKRNAKKWKELAREAMQEDGSSDKNLKAFVEDAGKGYQAESN

SEQ ID NO: 148-151 - skipped (4 entries)

SEQ ID NO: 152 - DNA, 1362 bp, Arabidopsis thaliana
atggaggaaaagcctgcaaggagaagcgtagtgttggttccatttccagcacaaggacatatatctccaatgatgcaacttgccaaaacccttcacttaaagggtttctcgatcacagttgttcagactaagttcaattactttagcccttcagatgacttcactcatgattttcagttcgtcaccattccagaaagcttaccagagtctgatttcaagaatctcggaccaatacagtttctgtttaagctcaacaaagagtgtaaggtgagcttcaaggactgtttgggtcagttggtgctgcaacaaagtaatgagatctcatgtgtcatctacgatgagttcatgtactttgctgaagctgcagccaaagagtgtaagcttccaaacatcattttcagcacaacaagtgccacggctttcgcttgccgctctgtatttgacaaactatatgcaaacaatgtccaagctcccttgaaagaaactaaaggacaacaagaagagctagttccggagttttatcccttgagatataaagactttccagtttcacggtttgcatcattagagagcataatggaggtgtataggaatacagttgacaaacggacagcttcctcggtgataatcaacactgcgagctgtctagagagctcatctctgtcttttctgcaacaacaacagctacaaattccagtgtatcctataggccctcttcacatggtggcctcagctcctacaagtctgcttgaagagaacaagagctgcatcgaatggttgaacaaacaaaaggtaaactcggtgatatacataagcatgggaagcatagctttaatggaaatcaacgagataatggaagtcgcgtcaggattggctgctagcaaccaacacttcttatgggtgatccgaccagggtcaatacctggttccgagtggatagagtccatgcctgaagagtttagtaagatggttttggaccgaggttacattgtgaaatgggctccacagaaggaagtactttctcatcctgcagtaggagggttttggagccattgtggatggaactcgacactagaaagcatcggccaaggagttccaatgatctgcaggccattttcgggtgatcaaaaggtgaacgctagatacttggagtgtgtatggaaaattgggattcaagtggagggtgagctagacagaggagtggtcgagagagctgtgaagaggttaatggttgacgaagaaggagaggagatgaggaagagagctttcagtttaaaagagcaacttagagcctctgttaaaagtggaggctcttcacacaactcgctagaagagtttgtacacttcataaggactgcctag

SEQ ID NO: 153 - protein, 453 aa, Arabidopsis thaliana
MEEKPARRSVVLVPFPAQGHISPMMQLAKTLHLKGFSITVVQTKFNYFSPSDDFTHDFQFVTIPESLPESDFKNLGPIQFLFKLNKECKVSFKDCLGQLVLQQSNEISCVIYDEFMYFAEAAAKECKLPNIIFSTTSATAFACRSVFDKLYANNVQAPLKETKGQQEELVPEFYPLRYKDFPVSRFASLESIMEVYRNTVDKRTASSVIINTASCLESSSLSFLQQQQLQIPVYPIGPLHMVASAPTSLLEENKSCIEWLNKQKVNSVIYISMGSIALMEINEIMEVASGLAASNQHFLWVIRPGSIPGSEWIESMPEEFSKMVLDRGYIVKWAPQKEVLSHPAVGGFWSHCGWNSTLESIGQGVPMICRPFSGDQKVNARYLECVWKIGIQVEGELDRGVVERAVKRLMVDEEGEEMRKRAFSLKEQLRASVKSGGSSHNSLEEFVHFIRTA

SEQ ID NO: 154-167 - skipped (14 entries)

SEQ ID NO: 168 - DNA, 1365 bp, Catharanthus roseus
atggcaactgaacaacaacaagcatctatctcctgcaaaatcttaatgtttccttggttagccttcggtcatatctcttctttcttacaattggctaagaaattgtctgatagaggtttctacttctacatttgtagtactccaattaatttggactctattaaaaataagataaaccaaaactattcttcatccatacaattggttgatttgcatttgccaaacagtcctcaattgccaccttctttacatactacaaatggtttgccacctcacttaatgtctacattgaaaaacgctttgatcgatgcaaatccagacttatgcaagattatagcctcaattaaaccagatttgatcatctatgacttacatcaaccttggaccgaagcattggcttctagacacaacattcctgctgttagtttttctactatgaatgccgtatcctttgcttacgttatgcacatgttcatgaatccaggtatagaatttcctttcaaagcaatccacttatcagattttgaacaagccagattcttggaacaattagaatcagctaagaacgatgcctccgctaaagacccagaattgcaaggtagtaagggtttctttaactctaccttcattgttagaagttctagagaaatcgagggtaaatacgttgattacttgtcagaaatcttaaagtccaaggtcattccagtatgtcctgttatatctttgaataacaacgatcaaggtcagggtaacaaagatgaagacgaaataatccaatggttagacaaaaagtctcatagatcatccgtatttgtttcattcggttccgaatactttttgaacatgcaagaaatcgaagaaatcgctataggtttggaattatctaacgtcaactttatatgggtattgagattcccaaagggtgaagatacaaaaattgaagaagttttgcctgaaggtttcttggacagagttaaaaccaagggtagaattgtccacggttgggcaccacaagccagaatcttgggtcatccttcaattggtggtttcgtatcccactgcggttggaatagtgttatggaatctatccaaatcggtgtcccaattatagcaatgcctatgaacttggatcaaccttttaatgccagattagttgtcgaaatcggtgtcggtattgaagtaggtagagatgaaaacggtaaattaaagagagaaagaatcggtgaagttatcaaggaagtcgctataggtaaaaagggtgaaaaattgagaaagacagcaaaagatttgggtcaaaaattgagagatagagaaaaacaagactttgacgaattagcagcaactttgaaacaattatgcgtatga

SEQ ID NO: 169 - protein, 454 aa, Catharanthus roseus
MATEQQQASISCKILMFPWLAFGHISSFLQLAKKLSDRGFYFYICSTPINLDSIKNKINQNYSSSIQLVDLHLPNSPQLPPSLHTTNGLPPHLMSTLKNALIDANPDLCKIIASIKPDLIIYDLHQPWTEALASRHNIPAVSFSTMNAVSFAYVMHMFMNPGIEFPFKAIHLSDFEQARFLEQLESAKNDASAKDPELQGSKGFFNSTFIVRSSREIEGKYVDYLSEILKSKVIPVCPVISLNNNDQGQGNKDEDEIIQWLDKKSHRSSVFVSFGSEYFLNMQEIEEIAIGLELSNVNFIWVLRFPKGEDTKIEEVLPEGFLDRVKTKGRIVHGWAPQARILGHPSIGGFVSHCGWNSVMESIQIGVPIIAMPMNLDQPFNARLVVEIGVGIEVGRDENGKLKRERIGEVIKEVAIGKKGEKLRKTAKDLGQKLRDREKQDFDELAATLKQLCV

SEQ ID NO: 170-171 - skipped (2 entries)

SEQ ID NO: 172 - DNA, 1362 bp, Arabidopsis thaliana
atgaccaaattctccgagccaatcagagactcccacgtggcagttctcgcgtttttccccgttggcgctcatgccggtcctctcttagccgtcactcgccgtctcgccgccgcttctccctccaccatcttttctttcttcaacaccgcaagatcaaacgcgtcgttgttctcctctgatcatcccgagaacatcaaggtccacgacgtctctgacggtgttccggagggaaccatgctcgggaatccactggagatggtcgagctgtttctcgaagcggctccacgtattttccggagcgaaatcgcggcggcagagatagaagttggaaagaaagtgacatgcatgctaacagatgccttcttctggttcgcagcggacatagcggctgagctgaacgcgacttgggttgccttctgggccggcggagcaaactcactctgtgctcatctctacactgatctcatcagagaaaccatcggtctcaaagatgtgagtatggaagagacattagggtttataccaggaatggagaattacagagttaaagatataccagaggaagttgtatttgaagatttggactctgttttcccaaaggctttataccaaatgagtcttgctttacctcgtgcctctgctgttttcatcagttcctttgaagagttagaacctacattgaactataacctaagatccaaacttaaacgtttcttgaacatcgcccctctcacgttattatcttctacatcggagaaagagatgcgtgatcctcatggctgctttgcttggatggggaagagatcagctgcttctgtagcgtacattagcttcggcaccgtcatggaacctcctcctgaagagcttgtggcgatagcacaagggttggaatcaagcaaagtgccgtttgtttggtcgctgaaggagaagaacatggttcatctaccaaaagggtttttggatcggacaagagagcaagggatagtggttccttgggctccacaagtggaactgctgaaacacgaggcaatgggtgtgaatgtgacacattgtggatggaactcagtgttggagagtgtgtcggcaggtgtaccgatgatcggcagaccgattttggcggataataggctcaacggaagagcagtggaggttgtgtggaaggttggagtgatgatggataatggagtcttcacgaaagaaggatttgagaagtgtttgaatgatgtttttgttcatgatgatggtaagacgatgaaggctaatgccaagaagcttaaagaaaaactccaagaagatttctccatgaaaggaagctctttagagaatttcaaaatattgttggacgaaattgtgaaagtttag

SEQ ID NO: 173 - protein, 453 aa, Arabidopsis thaliana
MTKFSEPIRDSHVAVLAFFPVGAHAGPLLAVTRRLAAASPSTIFSFFNTARSNASLFSSDHPENIKVHDVSDGVPEGTMLGNPLEMVELFLEAAPRIFRSEIAAAEIEVGKKVTCMLTDAFFWFAADIAAELNATWVAFWAGGANSLCAHLYTDLIRETIGLKDVSMEETLGFIPGMENYRVKDIPEEVVFEDLDSVFPKALYQMSLALPRASAVFISSFEELEPTLNYNLRSKLKRFLNIAPLTLLSSTSEKEMRDPHGCFAWMGKRSAASVAYISFGTVMEPPPEELVAIAQGLESSKVPFVWSLKEKNMVHLPKGFLDRTREQGIVVPWAPQVELLKHEAMGVNVTHCGWNSVLESVSAGVPMIGRPILADNRLNGRAVEVVWKVGVMMDNGVFTKEGFEKCLNDVFVHDDGKTMKANAKKLKEKLQEDFSMKGSSLENFKILLDEIVKV

SEQ ID NO: 174-175 - skipped (2 entries)

SEQ ID NO: 176 - DNA, 1275 bp, Streptomyces antibioticus
atgacttctgaacatagatccgcttccgttactccaagacatatttcattcttcaacatcccaggtcatggtcatgttaatccatctttgggtatcgttcaagaattggttgctagaggtcacagagtttcttacgctattaccgatgaatttgctgctcaagttaaggctgctggtgctactccagttgtttatgattccatcttgccaaaagaatccaacccagaagaatcttggccagaagatcaagaatctgctatgggtttgttcttggatgaagctgttagagtcttgccacaattagaagatgcttacgctgatgatagaccagatttgatcgtttacgatattgcttcttggccagctccagttttgggtagaaaatgggatattccattcgtccaattatccccaactttcgttgcttacgaaggttttgaagaagatgttccagcagttcaagatccaactgctgatagaggtgaagaagctgctgctccagcaggtactggtgatgctgaagaaggtgctgaagctgaagatggtttggttagattcttcactagattgtccgctttcttggaagaacatggtgttgatactccagctaccgaatttttgattgctccaaacagatgcatcgttgctttgccaagaacttttcaaatcaagggtgataccgttggtgataactacacttttgttggtccaacttacggtgatagatctcatcaaggtacttgggaaggtccaggtgatggtagaccagttttgttgattgctttgggttctgctttcactgatcacttggatttctacagaacctgtttgtctgctgttgatggtttggattggcatgttgttttgtctgttggtagatttgttgatccagcagatttgggtgaagttccaccaaatgttgaagttcatcaatgggttccacaattagatattttgaccaaggcttccgccttcattactcatgctggtatgggttctactatggaagccttgtctaatgctgttccaatggttgctgttccacaaattgctgaacaaactatgaacgccgaaagaatagtcgaattgggtttgggtagacatatcccaagagatcaagttactgccgaaaaattgagagaagctgttttggctgttgcttctgatccaggtgttgctgaaagattggctgctgttagacaagaaattagagaagccggtggtgctagagctgctgctgatattttggaaggtattttggctgaagccggttaa

SEQ ID NO: 177 - protein, 424 aa, Streptomyces antibioticus
MTSEHRSASVTPRHISFFNIPGHGHVNPSLGIVQELVARGHRVSYAITDEFAAQVKAAGATPVVYDSILPKESNPEESWPEDQESAMGLFLDEAVRVLPQLEDAYADDRPDLIVYDIASWPAPVLGRKWDIPFVQLSPTFVAYEGFEEDVPAVQDPTADRGEEAAAPAGTGDAEEGAEAEDGLVRFFTRLSAFLEEHGVDTPATEFLIAPNRCIVALPRTFQIKGDTVGDNYTFVGPTYGDRSHQGTWEGPGDGRPVLLIALGSAFTDHLDFYRTCLSAVDGLDWHVVLSVGRFVDPADLGEVPPNVEVHQWVPQLDILTKASAFITHAGMGSTMEALSNAVPMVAVPQIAEQTMNAERIVELGLGRHIPRDQVTAEKLREAVLAVASDPGVAERLAAVRQEIREAGGARAAADILEGILAEAG

SEQ ID NO: 178-179 - skipped (2 entries)

SEQ ID NO: 180 - DNA, 1437 bp, Oryza sativa
atgaagcaaaccgtcgtcctgtaccccggcggcggcgtcggccacgtcgtccccatgctggagctcgccaaggtcttcgtcaagcacgggcacgacgtcaccatggtgctgctggagccgcccttcaagtcgtccgactccggcgccctcgccgtcgagcgcctcgtcgcctccaacccttccgtctccttccacgtcctcccgccactccccgcccccgacttcgccagcttcggcaagcacccgttcctcctcgtcatccagctcctgcgccagtacaacgagcggctcgagagcttcctcctctccatccctcgacagcgcctgcactccctcgtcatcgacatgttctgcgtcgacgccatcgacgtgtgcgcaaagctcggcgtgccggtgtacacgttcttcgcctcgggcgtctcggtgctgtccgtcttgacccagctcccaccgtttcttgccggtagggagacgggcctgaaggagcttggcgacacgccgcttgatttcctcggtgtttcgccgatgccggcgtctcatctcgtcaaggaattgctcgagcatccggaggacgagttgtgcaaggccatggtgaaccgctgggagcgcaacacggaaaccatgggcgtcctggtgaactcgttcgaatcgttggagagccgggcggctcaggcgctcagggacgacccgctctgcgtcccaggcaaggtgctgcctccgatctactgcgtcgggcctttggtcggcggcggcgcggaggaggcggccgagaggcacgagtgcctcgtctggctcgacgctcagccggagcacagcgtcgtgttcctctgcttcgggagcaagggcgtgttctccgcggagcagctcaaggagatcgccgtcggcttggagaactccaggcaacggttcatgtgggtcgtgcgcacgccgccgacaaccaccgaaggcttgaagaagtacttcgagcaacgcgcggcgccggacctcgacgcgctcttcccggatgggttcgtggagcgtaccaaggaccgtggcttcatcgtcacgacgtgggcgccgcaggtggacgtgctccgccaccgggcgaccggcgcgttcgtgacgcactgcgggtggaactcggcgctggagggcatcacggcgggggtgccgatgctgtgctggccgcagtacgcggagcagaagatgaacaaggtgttcatgacggcggagatgggcgtcggggtggagctggacgggtacaactcggactttgtcaaagcggaggagttggaggccaaggtgaggctggtgatggagtcggaggaagggaagcagctcagggctcgttcggctgcgcggaagaaggaggcagaggcggcgctggaggaagggggctcgtcgcacgctgcgttcgtccagttcctgtccgatgtggagaatcttgtccagaactaa

SEQ ID NO: 181 - protein, 478 aa, Oryza sativa
MKQTVVLYPGGGVGHVVPMLELAKVFVKHGHDVTMVLLEPPFKSSDSGALAVERLVASNPSVSFHVLPPLPAPDFASFGKHPFLLVIQLLRQYNERLESFLLSIPRQRLHSLVIDMFCVDAIDVCAKLGVPVYTFFASGVSVLSVLTQLPPFLAGRETGLKELGDTPLDFLGVSPMPASHLVKELLEHPEDELCKAMVNRWERNTETMGVLVNSFESLESRAAQALRDDPLCVPGKVLPPIYCVGPLVGGGAEEAAERHECLVWLDAQPEHSVVFLCFGSKGVFSAEQLKEIAVGLENSRQRFMWVVRTPPTTTEGLKKYFEQRAAPDLDALFPDGFVERTKDRGFIVTTWAPQVDVLRHRATGAFVTHCGWNSALEGITAGVPMLCWPQYAEQKMNKVFMTAEMGVGVELDGYNSDFVKAEELEAKVRLVMESEEGKQLRARSAARKKEAEAALEEGGSSHAAFVQFLSDVENLVQN

SEQ ID NO: 182 - DNA, 1380 bp, Nicotiana tabacum
atgactactcaaaaagctcattgcttgatcttaccatatccagctcagggtcatatcaaccctatgctccaattctccaaacgtttgcaatccaaaggtgtcaaaatcactatagcagccaccaaatcattcttgaaaaccatgcaagaattgtcaacttctgtgtcagtcgaggctatctccgatggctatgatgatggcggacgcgagcaagctggaacctttgtggcctatattacaagattcaaagaagttggctcggatactttgtctcagcttattggaaagttaacaaattgtggttgtcctgtgagttgcatagtttacgatccatttcttccttgggctgttgaagtgggaaataattttggagtagctactgctgcttttttcactcaatcttgtgcagtggataacatttattaccatgtacataaaggggttctaaaacttcctccaactgacgttgataaagaaatctcaattcctggattattaacaattgaggcatcagatgtacctagttttgtttctaatcctgaatcttcaagaatacttgaaatgttggtgaatcagttctcgaatcttgagaacacagattgggtcctaatcaacagtttctatgaattggagaaagaggtaattgattggatggccaagatctatccaatcaagacaattggaccaactataccatcaatgtacctagacaagaggctaccagatgacaaagaatatggccttagtgtcttcaagccaatgacaaatgcatgcctaaactggttaaaccatcaaccagttagctcagtagtatatgtatcatttggaagtttagccaaattagaagcagagcaaatggaagaattagcatggggtttgagtaatagcaacaagaacttcttgtgggtagttagatccactgaagaatccaaacttcccaacaactttttagaggaattagcaagtgaaaaaggattagtcgtgtcatggtgtccacaattacaagtcttggaacataaatcaatagggtgttttctcacgcactgtggctggaattcaactttggaagcaattagtttgggagtaccaatgattgcaatgccacattggtcagaccagccaacaaatgcgaagcttgtggaagatgtttgggagatgggaattagaccaaaacaagatgaaaaaggattagttagaagagaagttattgaagaatgtattaagatagtgatggaggaaaagaaaggaaaaaagattagggaaaatgcaaagaaatggaaggaattggctaggaaagctgtggatgaaggaggaagttcagatagaaatattgaagaatttgtttccaagttggtgactattgcctcagtggaaagctaa

SEQ ID NO: 183 - protein, 459 aa, Nicotiana tabacum
MTTQKAHCLILPYPAQGHINPMLQFSKRLQSKGVKITIAATKSFLKTMQELSTSVSVEAISDGYDDGGREQAGTFVAYITRFKEVGSDTLSQLIGKLTNCGCPVSCIVYDPFLPWAVEVGNNFGVATAAFFTQSCAVDNIYYHVHKGVLKLPPTDVDKEISIPGLLTIEASDVPSFVSNPESSRILEMLVNQFSNLENTDWVLINSFYELEKEVIDWMAKIYPIKTIGPTIPSMYLDKRLPDDKEYGLSVFKPMTNACLNWLNHQPVSSVVYVSFGSLAKLEAEQMEELAWGLSNSNKNFLWVVRSTEESKLPNNFLEELASEKGLVVSWCPQLQVLEHKSIGCFLTHCGWNSTLEAISLGVPMIAMPHWSDQPTNAKLVEDVWEMGIRPKQDEKGLVRREVIEECIKIVMEEKKGKKIRENAKKWKELARKAVDEGGSSDRNIEEFVSKLVTIASVES

SEQ ID NO: 184 - DNA, 1365 bp, Siraitia grosvenorii
atggagaaaggcgatacgcatattctagtgtttcctttcccttcacaaggccacataaaccctcttcttcaactatcgaagcgcctaatcgccaagggaatcaaggtttcgctggtcacaaccttacatgttagcaatcacttgcagttgcagggtgcttattccaactccgtgaagatcgaagtcatttccgatggctctgaggatcgtctggaaaccgatactatgcgccaaactctggatcgatttcggcagaagatgacgaagaacttggaagatttcttgcagaaagccatggtttcttcaaatccgcctaaattcattctgtatgattcgacaatgccgtgggttttggaggtcgccaaggagttcggactcgatagggccccgttctacactcagtcttgtgcgcttaacagtatcaattatcatgttcttcatggtcaattgaagcttcctcctgaaacccccacgatttcgttgccttctatgcctctgcttcgccccagcgatctcccggcttatgattttgatcctgcctccactgacaccatcatcgatcttcttaccagtcagtattctaatatccaggatgcaaatctgcttttctgcaacacttttgacaagttggaaggcgagattatccaatggatggagaccctgggtcgccctgtgaaaaccgtaggaccaactgttccatcagcctacttagacaaaagggtagagaacgacaagcactatgggctgagtctgttcaagcccaacgaggacgtctgcctcaaatggcttgatagcaagccctctggttctgttctgtatgtgtcttatggcagtttggttgaaatgggggaagagcagctgaaggagttggctctgggaatcaaggaaactggcaagttcttcttgtgggtggtgagagacactgaagcagagaagcttcctcccaactttgtggagagtgtggcagagaaggggcttgtggtcagctggtgctcccagctggaggtattggctcacccctccgtcggctgcttcttcacgcactgtggctggaactcgacgcttgaggcgctgtgcttgggcgtcccggtggtcgctttcccacagtgggctgatcaggtaaccaatgcaaagtttttagaagatgtttggaaggttgggaagagggtgaagcggaatgagcagaggctggcaagtaaagaagaagtaaggagttgcatttgggaagtgatggagggagagagagccagcgagttcaagagcaactccatggagtggaagaagtgggcaaaagaagctgtggatgaaggtgggagctctgataagaacattgaggagtttgtggctatgctcaagcaaacttga

SEQ ID NO: 185 - protein, 454 aa, Siraitia grosvenorii
MEKGDTHILVFPFPSQGHINPLLQLSKRLIAKGIKVSLVTTLHVSNHLQLQGAYSNSVKIEVISDGSEDRLETDTMRQTLDRFRQKMTKNLEDFLQKAMVSSNPPKFILYDSTMPWVLEVAKEFGLDRAPFYTQSCALNSINYHVLHGQLKLPPETPTISLPSMPLLRPSDLPAYDFDPASTDTIIDLLTSQYSNIQDANLLFCNTFDKLEGEIIQWMETLGRPVKTVGPTVPSAYLDKRVENDKHYGLSLFKPNEDVCLKWLDSKPSGSVLYVSYGSLVEMGEEQLKELALGIKETGKFFLWVVRDTEAEKLPPNFVESVAEKGLVVSWCSQLEVLAHPSVGCFFTHCGWNSTLEALCLGVPVVAFPQWADQVTNAKFLEDVWKVGKRVKRNEQRLASKEEVRSCIWEVMEGERASEFKSNSMEWKKWAKEAVDEGGSSDKNIEEFVAMLKQT

SEQ ID NO: 186-197 - skipped (12 entries)

SEQ ID NO: 198 - DNA, 1446 bp, Crocus sativus
atggggtcagaagataggtccttgtccatcttattctttccttttatggcacaaggtcacatgttacctatgctagatatggctaagttatttgctctgtatggtgtcaaatcaacagtagtgaccactccagctaatgtaccaatagtcaactcagtaattgatcagcctgatgtttctactttgcacccaatccaattacgactgataccatttccatctgacacgggcttgcctgaaggttgtgaaaacgtatcatcaattcctccaagagacatgccaactgttcatgtcactttcttcagcgctacagcaaaacttagagaaccttttggtaaggtgctagaggatctaagaccagattgtattgttactgacatgtttttcccttggacctacgatgtggccgcagaattaggtatcccaaggattgttttccatgggacaaatttcttttctctctgcgtaacagattctcttgaaagatataaaccagttgaaaacttgcgaagtgatgccgagtctgtagtgatcccaggactcccacacagaatcgaggtattgcgttctcaaataccagaatacgaaaaatcaaaagcagattttgttagagaagttagggaatcagaatctaagtcttacggagcggtggttaattctttctttgaattggaacctgactacgctagacattacagagaggttgtcggcagacgtgcttggcatatcgggccacttgctctggtcaataactctactacagacaaaagctcaagaggatacaagacagcgatcgatagaaacgattgtttgaaatggctcgattctaaaagactaagatccgttgtatatgtgtgctttggctcaatgtctgacttttccgatgcccaattacgtgaaatggcaagtggtctagaggcatccaatcatcctttcatttgggtggttagaaaatctggcaaggaatggttaccagaaggatttgaggaaagagtccaggagagaggtttgattatcagaggctgggctccacaaatcttaatactcaaccatagagcagtgggaggcttcatgacccattgtgggtggaatagtagtttggaagcagtttctgccggactgcctcttgttacatggcctctatttgcagaacaattttacaatgaaagattcatggttgatgttttgagaattggtgtatcagtgggtgcgaagagacacggtatgaaagccgaagagagagaagtcgtagaagccaaaatggttaaggaagctgttgatggcttgatggacgacggtgaagaggctgagggtagaaggcgtagagctagagaactgggcgaaaaagctagaaaggccgtcgaaaaaggtggttcatcctacgaggacatgagaaatcttttgcaagagcttaagggtgatagcaagttaactgtcggatgctaa

SEQ ID NO: 199 - protein, 481 aa, Crocus sativus
MGSEDRSLSILFFPFMAQGHMLPMLDMAKLFALYGVKSTVVTTPANVPIVNSVIDQPDVSTLHPIQLRLIPFPSDTGLPEGCENVSSIPPRDMPTVHVTFFSATAKLREPFGKVLEDLRPDCIVTDMFFPWTYDVAAELGIPRIVFHGTNFFSLCVTDSLERYKPVENLRSDAESVVIPGLPHRIEVLRSQIPEYEKSKADFVREVRESESKSYGAVVNSFFELEPDYARHYREVVGRRAWHIGPLALVNNSTTDKSSRGYKTAIDRNDCLKWLDSKRLRSVVYVCFGSMSDFSDAQLREMASGLEASNHPFIWVVRKSGKEWLPEGFEERVQERGLIIRGWAPQILILNHRAVGGFMTHCGWNSSLEAVSAGLPLVTWPLFAEQFYNERFMVDVLRIGVSVGAKRHGMKAEEREVVEAKMVKEAVDGLMDDGEEAEGRRRRARELGEKARKAVEKGGSSYEDMRNLLQELKGDSKLTVGC

SEQ ID NO: 200 - DNA, 1395 bp, Crocus sativus
atggaggctggaggtgacaaacttcacattgttgtctttccatggttagcttttggccacatgttgccatttctagagctgtctaagtctttggctaaaagaggtcacttaatcagttttgtttctacacctaaaaacattcaaagatttcctaatcttccaccacaaatctcaccacttatcaactttatcccattaagtctacctaaagtggagggcatgccaggtgacgtagaagctaccacagacctaccacctgccaacctacaatatctgaaaaaggcacttgacgggttagaacaacctttcagatcattcctaagagaggcctccccaaaacctgattggataatccaagatcttttacaacattggatacctccaattgccgcagaacttcatgttccttccatgtactttggcacagtgccagctgccgccttgacctttttcggtcatccatcacaacttagttcaagagggaagggattggaaggctggctggcttcaccaccatgggttccattcccatctaaggtggcatacagattgcacgaactaatcgttatggctaaagatgccgctggtccattgcattccggtatgactgatgctagaaggatggaagctgcaatagttggatgctgtgcagtcgctattagaacatgtagagaattggaatcagaatggttacctattctggaggagatctacggaaagcctgtgataccagttggattacttttacctactgctgatgaatctactgatggaaactctatcatagactggttaggcacaagatcccaggaatcagtagtgtacattgctctgggttcagaagtttctattggtgtggaattgatacatgaattggccttgggtcttgaattagcaggtttgccattcctatgggcactacgtagaccttatggactgtctagtgatactgagattttgcctggtggattcgaggagagaactagaggctatggaaaggtagtcatgggctgggttcctcaaatgagagtcttggcagatcgttctgtaggcggctttgtcacacactgtggttggtcatctgtagttgaatcattacattttgggcatccactagttttactgccaatcttcggtgaccaaggattgaatgcaagattgctggaggaaaagggaattggggtcgaagtagaaaggaagggtgatgggtcttttacccgtaatgaagttgcaaaagcaatcaatttgatcatggtcgaaggtgacggttctggttcctcctacaggaaaaaggcaaaggaaatgaaaaagattttcgctgataaggaatgccaggagaaatacgtggatgaatttgtgcagttcctgttatcaaatggtactgctaaaggctaa

SEQ ID NO: 201 - protein, 464 aa, Crocus sativus
MEAGGDKLHIVVFPWLAFGHMLPFLELSKSLAKRGHLISFVSTPKNIQRFPNLPPQISPLINFIPLSLPKVEGMPGDVEATTDLPPANLQYLKKALDGLEQPFRSFLREASPKPDWIIQDLLQHWIPPIAAELHVPSMYFGTVPAAALTFFGHPSQLSSRGKGLEGWLASPPWVPFPSKVAYRLHELIVMAKDAAGPLHSGMTDARRMEAAIVGCCAVAIRTCRELESEWLPILEEIYGKPVIPVGLLLPTADESTDGNSIIDWLGTRSQESVVYIALGSEVSIGVELIHELALGLELAGLPFLWALRRPYGLSSDTEILPGGFEERTRGYGKVVMGWVPQMRVLADRSVGGFVTHCGWSSVVESLHFGHPLVLLPIFGDQGLNARLLEEKGIGVEVERKGDGSFTRNEVAKAINLIMVEGDGSGSSYRKKAKEMKKIFADKECQEKYVDEFVQFLLSNGTAKG

SEQ ID NO: 202 - DNA, 1350 bp, Arabidopsis thaliana
atggagaagatgagaggacatgtattagcagtgccatttccaagccaaggacacatcaccccgattcgccaattctgcaaacgacttcactccaaaggtttcaaaaccactcacactctcaccacttttatcttcaacacaatccacctcgacccatctagtcctatctccatagccacaatctccgatggctatgaccagggagggttctcatcagccggttctgtcccggagtacctacaaaacttcaaaaccttcggctccaaaaccgtcgctgatatcatccgcaaacaccagagtactgataaccctattacttgtatcgtctatgattctttcatgccttgggcgcttgaccttgcaatggattttggtctagctgcggctcctttcttcacgcagtcttgcgccgttaactatatcaattatctttcttacataaacaatggtagcttgacacttcccatcaaggatttgcctcttcttgagctccaagatttgcctactttcgtcactcctactggttcacaccttgcttactttgagatggtgcttcaacagttcaccaacttcgacaaagctgatttcgtactcgttaattccttccatgacctcgaccttcatgaagaggagttgttgtcgaaagtatgtcctgtgttgacaattggtccaactgttccatcaatgtacttagaccaacagatcaaatcagacaacgactatgatctgaacctctttgacttaaaagaagctgccttatgcactgactggctagacaagaggccagaaggatcggtagtatatatagcttttgggagcatggctaaactgagtagtgagcagatggaagagattgcttcggcgataagcaacttcagctacctctgggttgtcagagcttcagaggagtcaaagctcccaccagggtttcttgaaacagtggataaagacaagagcttggtcttgaagtggagtcctcagcttcaagttctgtcaaacaaagccatcggttgtttcatgactcactgtggctggaactcaaccatggagggtttgagtttaggggttcccatggtggctatgcctcaatggactgatcaaccaatgaatgcaaagtatatacaagatgtatggaaggttggggttcgtgtgaaagcagagaaagaaagtggcatttgcaaaagagaggagattgagtttagcatcaaggaagtgatggaaggagagaagagcaaagagatgaaagagaatgcgggaaaatggagagacttggctgtgaagtcactcagtgaaggaggttctacagatatcaacattaacgaatttgtatcaaaaattcaaatcaaataa

SEQ ID NO: 203 - protein, 449 aa, Arabidopsis thaliana
MEKMRGHVLAVPFPSQGHITPIRQFCKRLHSKGFKTTHTLTTFIFNTIHLDPSSPISIATISDGYDQGGFSSAGSVPEYLQNFKTFGSKTVADIIRKHQSTDNPITCIVYDSFMPWALDLAMDFGLAAAPFFTQSCAVNYINYLSYINNGSLTLPIKDLPLLELQDLPTFVTPTGSHLAYFEMVLQQFTNFDKADFVLVNSFHDLDLHEEELLSKVCPVLTIGPTVPSMYLDQQIKSDNDYDLNLFDLKEAALCTDWLDKRPEGSVVYIAFGSMAKLSSEQMEEIASAISNFSYLWVVRASEESKLPPGFLETVDKDKSLVLKWSPQLQVLSNKAIGCFMTHCGWNSTMEGLSLGVPMVAMPQWTDQPMNAKYIQDVWKVGVRVKAEKESGICKREEIEFSIKEVMEGEKSKEMKENAGKWRDLAVKSLSEGGSTDININEFVSKIQIK

SEQ ID NO: 204 - DNA, 1425 bp, Arabidopsis thaliana
atggccaacaacaattccaactctcccaccggtccacactttctattcgtaacatttccagcccaaggtcacatcaacccatctctcgagctagccaaacgcctcgccggaacaatctctggtgctcgagtcaccttcgccgcctcaatctctgcctacaaccgccgcatgttctctacagaaaacgtccccgaaaccctaatcttcgctacctactccgatggccacgacgacggtttcaaatcctctgcttactccgacaaatctcgtcaagacgccactggaaacttcatgtctgagatgagacgacgtggcaaagagacactaaccgaactaatcgaagataaccggaaacaaaacaggccttttacttgcgtggtttacacgattctcctcacttgggtcgctgagctagcgcgtgagtttcatcttccttctgctcttctttgggtccaaccagtaacagtcttctccattttttaccattacttcaatggctacgaagatgcaatctcagagatggctaataccccctctagttctattaaattaccttctctgccactgcttactgtccgtgatattccttctttcattgtctcttccaatgtctacgcgtttcttctacccgcgtttcgagaacagattgattcactgaaggaagaaataaaccctaagatcctcatcaacactttccaagagcttgagccagaagccatgagctcggttccagataatttcaagattgtccctgtcggtccgttactaacgttgagaacggatttttcgagtcgcggtgaatacatagagtggttggatactaaagcggattcgtctgtgctttatgtttcgttcgggacgcttgccgtgttgagcaagaaacagcttgtggagctttgtaaagcgttgatacaaagtcggagaccattcttgtgggtgattacggataagtcgtacagaaataaagaagatgagcaagagaaggaagaagattgcataagtagtttcagagaagagctcgatgagataggaatggtggtttcatggtgtgatcagtttagggttttgaatcatagatcgataggttgtttcgtgacgcattgcgggtggaactctacgctggagagcttggtttcaggagttccggtggtggcgtttccgcaatggaatgatcagatgatgaacgcgaagcttttagaagattgttggaaaacaggtgtaagagtgatggagaagaaggaagaagaaggagttgtggtggtggatagtgaggagatacggcggtgcattgaggaagttatggaagacaaggcggaggagtttagaggaaatgccacgaggtggaaggatttagcggcggaggctgtgagagaaggaggctcttcctttaatcatctcaaagcttttgtcgatgagcacatctag

SEQ ID NO: 205 - protein, 474 aa, Arabidopsis thaliana
MANNNSNSPTGPHFLFVTFPAQGHINPSLELAKRLAGTISGARVTFAASISAYNRRMFSTENVPETLIFATYSDGHDDGFKSSAYSDKSRQDATGNFMSEMRRRGKETLTELIEDNRKQNRPFTCVVYTILLTWVAELAREFHLPSALLWVQPVTVFSIFYHYFNGYEDAISEMANTPSSSIKLPSLPLLTVRDIPSFIVSSNVYAFLLPAFREQIDSLKEEINPKILINTFQELEPEAMSSVPDNFKIVPVGPLLTLRTDFSSRGEYIEWLDTKADSSVLYVSFGTLAVLSKKQLVELCKALIQSRRPFLWVITDKSYRNKEDEQEKEEDCISSFREELDEIGMVVSWCDQFRVLNHRSIGCFVTHCGWNSTLESLVSGVPVVAFPQWNDQMMNAKLLEDCWKTGVRVMEKKEEEGVVVVDSEEIRRCIEEVMEDKAEEFRGNATRWKDLAAEAVREGGSSFNHLKAFVDEHI

SEQ ID NO: 206 - DNA, 1353 bp, Arabidopsis thaliana
atgggaagtaatgagggtcaagaaacacatgtcctaatggtagcattagcattccaaggtcatctcaatccaatgctcaaattcgcaaaacatctcgcacgaaccaatctacacttcactctcgccaccactgagcaagcccgtgacctcctctcttccaccgctgacgaacctcatagaccggtggacctcgctttcttctcagacggtctacctaaagacgatccaagagatcccgacactctcgcaaagtcattgaaaaaagatggagccaagaacttgtcaaaaatcatcgaagaaaagagatttgattgcatcatctctgtgccttttactccctgggttccagctgttgcagctgcacataacattccttgtgcaatcctctggatccaagcttgtggagctttttctgtttattaccgttattacatgaagacaaatcctttccccgaccttgaagatctgaatcaaacagtggagttaccagctttaccattgttggaagtccgagatctcccgtcattgatgttaccttctcaaggagctaatgtcaataccctaatggcggaatttgcagattgtttgaaagatgtgaaatgggttttggttaactcgttttacgaactcgaatcagagatcatcgagtctatgtctgatttaaaacctataatcccaattggtcctcttgtttctccattcctgttgggaaatgatgaagaaaaaaccctagatatgtggaaagttgatgattattgtatggagtggcttgacaagcaagctaggtcttcagttgtttacatatctttcggaagcatactcaaatcattggagaatcaagttgagaccatagcaacggcattaaaaaacagaggagttccatttctttgggtgatacggccgaaggagaaaggcgaaaacgtccaggttttgcaggagatggttaaagaaggtaaaggggttgtaactgaatggggtcaacaagaaaagatattgagccacatggcgatttcttgcttcatcacgcattgtggatggaactcgacgatcgagacggtggtgactggtgttcccgtggtggcgtatccgacttggatagatcagccgcttgatgcgagactgcttgtggatgtgtttggaatcggagtaaggatgaagaacgacgctatcgatggagagcttaaggttgcagaggtggagagatgcattgaggccgtgacagagggacctgccgccgcggatatgaggaggagagcgacggagctgaagcacgccgcaagatcggcgatgtcacctggtggatcttccgctcagaatttagactcgttcattagtgatatcccaatcacttga

SEQ ID NO: 207 - protein, 450 aa, Arabidopsis thaliana
MGSNEGQETHVLMVALAFQGHLNPMLKFAKHLARTNLHFTLATTEQARDLLSSTADEPHRPVDLAFFSDGLPKDDPRDPDTLAKSLKKDGAKNLSKIIEEKRFDCIISVPFTPWVPAVAAAHNIPCAILWIQACGAFSVYYRYYMKTNPFPDLEDLNQTVELPALPLLEVRDLPSLMLPSQGANVNTLMAEFADCLKDVKWVLVNSFYELESEIIESMSDLKPIIPIGPLVSPFLLGNDEEKTLDMWKVDDYCMEWLDKQARSSVVYISFGSILKSLENQVETIATALKNRGVPFLWVIRPKEKGENVQVLQEMVKEGKGVVTEWGQQEKILSHMAISCFITHCGWNSTIETVVTGVPVVAYPTWIDQPLDARLLVDVFGIGVRMKNDAIDGELKVAEVERCIEAVTEGPAAADMRRRATELKHAARSAMSPGGSSAQNLDSFISDIPIT

SEQ ID NO: 208 - DNA, 1464 bp, Catharanthus roseus
atggttaatcagctccatattttcaacttcccattcatggcacagggccatatgttacccgccttagacatggccaatctattcacttctcgtggagtcaaagtaacattaatcacaacccatcaacatgttcccatgtttacaaaatccatagaaaggagcagaaattctggatttgatatatccattcaatccatcaaattcccagcttcagaagttggtttacctgaaggaatcgaaagtctagatcaagtttcaggggacgacgaaatgcttcctaagttcatgagaggagttaatttactccaacaacctctcgaacaactattgcaagaatctcgtcctcattgtcttctttctgatatgttcttcccttggactactgaatctgctgctaaatttggtattcccagattgctttttcatgggtcctgttcctttgccctctctgcagctgaaagtgtgagaagaaataaacctttcgagaatgtttccacagacacagaggaatttgttgtgcctgatcttccccaccaaattaaattaaccagaacacaaatttcaacatacgaaagggaaaatattgagtcagattttaccaaaatgctgaagaaagttagggattcagaatccacatcttacggagttgtagtcaatagtttctatgaacttgaaccagattatgccgattattacatcaacgttttgggaagaaaagcatggcatatagggccttttttgctttgtaacaaatcacgagctgaagataaagcccaaagggggaagaaatcagcaattgatgcagacgaatgtttaaattggcttgattcgaaacaaccaaattccgtaatttatctctgtttcggaagtatggccaatttaaattctgcccaattacacgaaattgcaacagcccttgaatcctccggccaaaatttcatctgggttgttagaaaatgtgtggacgaagaaaacagttcaaaatggtttccagaaggattcgaagaaagaacaaaagaaaaagggctaattataaagggatgggcaccacaaaccctaattcttgaacacgaatcagtaggagcatttgttacccattgtggttggaattcaactcttgaaggaatctgcgcaggggttcctctggtgacttggcctttctttgctgagcaatttttcaatgagaaattgattacagaggtactgaaaacgggatacggagttggggctcggcaatggagtagagtttcaacagagattataaaaggagaagccatagctaatgctattaatcgagtaatggtgggtgatgaagctgttgagatgagaaacagagcaaaagatttgaaggaaaaggcaagaaaagctttggaagaagatggatcttcttatcgtgatcttactgctcttattgaagaattgggggcatatcgttctcaagttgaaagaaagcaacaagactag

SEQ ID NO: 209 - protein, 487 aa, Catharanthus roseus
MVNQLHIFNFPFMAQGHMLPALDMANLFTSRGVKVTLITTHQHVPMFTKSIERSRNSGFDISIQSIKFPASEVGLPEGIESLDQVSGDDEMLPKFMRGVNLLQQPLEQLLQESRPHCLLSDMFFPWTTESAAKFGIPRLLFHGSCSFALSAAESVRRNKPFENVSTDTEEFVVPDLPHQIKLTRTQISTYERENIESDFTKMLKKVRDSESTSYGVVVNSFYELEPDYADYYINVLGRKAWHIGPFLLCNKSRAEDKAQRGKKSAIDADECLNWLDSKQPNSVIYLCFGSMANLNSAQLHEIATALESSGQNFIWVVRKCVDEENSSKWFPEGFEERTKEKGLIIKGWAPQTLILEHESVGAFVTHCGWNSTLEGICAGVPLVTWPFFAEQFFNEKLITEVLKTGYGVGARQWSRVSTEIIKGEAIANAINRVMVGDEAVEMRNRAKDLKEKARKALEEDGSSYRDLTALIEELGAYRSQVERKQQD

SEQ ID NO: 210 - DNA, 1371 bp, Solanum lycopersicum
atgactactcacaaagctcattgcttaattttgccatttccaggccaaggtcatatcaacccaatgcttcaattctccaaacgtttacaatccaaacgcgttaaaatcactatagcactcacaaaatcctgtttgaaaacaatgcaagaattgtcaacttcagtatcaatcgaggcgatttctgatggctacgatgatggtggtttccatcaagcagaaaatttcgtagcctacataacacgattcaaagaagttggttcggatactctgtctcagcttattaaaaaattggaaaatagtgattgtcctgtaaattgcatagtatatgatccattcattccttgggctgttgaagttgcaaaacaatttggattaattagtgctgcatttttcacacaaaattgtgtagtggataatctttattaccatgtacataaaggggtgataaaacttccacctactcaaaatgacgaagaaatattaattcctggatttccaaattcgatcgatgcatcagatgtaccttcttttgttattagtcctgaagcagaaaggatagttgaaatgttagcaaatcaattctcaaatcttgacaaagttgattatgttctaatcaatagcttctatgagttggagaaagaggtaaatgaatggatgtcaaagatatatccaataaagacaattggaccaacaataccatcaatgtacttagacaagagactacatgatgataaagagtatggtcttagtgtcttcaagccaatgacaaatgaatgtctaaattggttaaaccatcaaccaattagctcagtggtgtatgtatcatttggaagtataaccaaattaggagatgagcaaatggaagaattggcatggggtttgaagaatagcaacaagagcttcttgtgggttgttaggtctactgaagagcccaaacttcccaacaactttattgaggaattaacaagtgaaaaaggcttagtggtgtcatggtgtccacaattacaagtgttggaacatgaatcgacaggttgttttctgacgcactgtggatggaattcaactctggaagcgattagtttgggagtgccaatggtggcaatgccacaatggtctgatcaaccaacaaatgcaaagcttgtgaaagatgtttgggaaataggtgttagagccaaacaagatgaaaaaggggtagttagaagagaagttatagaagaatgtataaagctagtgatggaagaagataaaggaaaactaattagagaaaatgcaaagaaatggaaggaaatagctagaaatgttgtgaatgaaggaggaagttcagataaaaacattgaagaatttgtttccaagttggttactatttcctaa

SEQ ID NO: 211 - protein, 456 aa, Solanum lycopersicum
MTTHKAHCLILPFPGQGHINPMLQFSKRLQSKRVKITIALTKSCLKTMQELSTSVSIEAISDGYDDGGFHQAENFVAYITRFKEVGSDTLSQLIKKLENSDCPVNCIVYDPFIPWAVEVAKQFGLISAAFFTQNCVVDNLYYHVHKGVIKLPPTQNDEEILIPGFPNSIDASDVPSFVISPEAERIVEMLANQFSNLDKVDYVLINSFYELEKEVNEWMSKIYPIKTIGPTIPSMYLDKRLHDDKEYGLSVFKPMTNECLNWLNHQPISSVVYVSFGSITKLGDEQMEELAWGLKNSNKSFLWVVRSTEEPKLPNNFIEELTSEKGLVVSWCPQLQVLEHESTGCFLTHCGWNSTLEAISLGVPMVAMPQWSDQPTNAKLVKDVWEIGVRAKQDEKGVVRREVIEECIKLVMEEDKGKLIRENAKKWKEIARNVVNEGGSSDKNIEEFVSKLVTIS

SEQ ID NO: 212 - DNA, 1422 bp, Artificial Sequence
  Codon-optimized UGT91D2e-b
atggctaccagtgactccatagttgacgaccgtaagcagcttcatgttgcgacgttcccatggcttgctttcggtcacatcctcccttaccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattccatatctcaagaaggcttctgatggtcttcaaccggaggtcacccggtttctagaacaacactctccggactggattatttatgattatactcactactggttgccatccatcgcggctagcctcggtatctcacgagcccacttctccgtcaccactccatgggccattgcttatatgggaccctcagctgacgccatgataaatggttcagatggtcgaaccacggttgaggatctcacgacaccgcccaagtggtttccctttccgaccaaagtatgctggcggaagcatgatcttgcccgactggtgccttacaaagctccggggatatctgatggataccgtatggggatggttcttaagggatctgattgtttgctttccaaatgttaccatgagtttggaactcaatggctacctcttttggagacactacaccaagtaccggtggttccggtgggattactgccaccggaaatacccggagacgagaaagatgaaacatgggtgtcaatcaagaaatggctcgatggtaaacaaaaaggcagtgtggtgtacgttgcattaggaagcgaggctttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccatgagtcggtttgtggtttcttgactcattgtggttctggatcaattgtggaagggctaatgtttggtcaccctctaatcatgctaccgatttttggggaccaacctctgaatgctcgattactggaggacaaacaggtgggaatcgagataccaagaaatgaggaagatggttgcttgaccaaggagtcggttgctagatcactgaggtccgttgttgtggaaaaagaaggggagatctacaaggcgaacgcgagggagctgagtaaaatctataacgacactaaggttgaaaaagaatatgtaagccaattcgtagactatttggaaaagaatgcgcgtgcggttgccatcgatcatgagagttaa

SEQ ID NO: 213 - DNA, 1383 bp, Stevia rebaudiana
atggcggaacaacaaaagatcaagaaatcaccacacgttctactcatcccattccctttacaaggccatataaaccctttcatccagtttggcaaacgattaatctccaaaggtgtcaaaacaacacttgttaccaccatccacaccttaaactcaaccctaaaccacagtaacaccaccaccacctccatcgaaatccaagcaatttccgatggttgtgatgaaggcggttttatgagtgcaggagaatcatatttggaaacattcaaacaagttgggtctaaatcactagctgacttaatcaagaagcttcaaagtgaaggaaccacaattgatgcaatcatttatgattctatgactgaatgggttttagatgttgcaattgagtttggaatcgatggtggttcgtttttcactcaagcttgtgttgtaaacagcttatattatcatgttcataagggtttgatttctttgccattgggtgaaactgtttcggttcctggatttccagtgcttcaacggtgggagacaccgttaattttgcagaatcatgagcaaatacagagcccttggtctcagatgttgtttggtcagtttgctaatattgatcaagcacgttgggtcttcacaaatagtttttacaagctcgaggaagaggtaatagagtggacgagaaagatatggaacttgaaggtaatcgggccaacacttccatccatgtaccttgacaaacgacttgatgatgataaagataacggatttaatctctacaaagcaaaccatcatgagtgcatgaactggttagacgataagccaaaggaatcagttgtttacgtagcatttggtagcctggtgaaacatggacccgaacaagtggaagaaatcacacgggctttaatagatagtgatgtcaacttcttgtgggttatcaaacataaagaagagggaaagctcccagaaaatctttcggaagtaataaaaaccggaaagggtttgattgtagcatggtgcaaacaattggatgtgttagcacacgaatcagtaggatgctttgttacacattgtgggttcaactcaactcttgaagcaataagtcttggagtccccgttgttgcaatgcctcaattttcggatcaaactacaaatgccaagcttctagatgaaattttgggtgttggagttagagttaaggctgatgagaatgggatagtgagaagaggaaatcttgcgtcatgtattaagatgattatggaggaggaaagaggagtaataatccgaaagaatgcggtaaaatggaaggatttggctaaagtagccgttcatgaaggtggtagctcagacaatgatattgtcgaatttgtaagtgagctaattaaggcttaa